(19) United States
(12) Patent Application Publication
(10) Pub. No.: US 2021/0238260 A1
(43) Pub. Date: Aug. 5, 2021

(54) GENE THERAPY FOR HEMOPHILIA B WITH A CHIMERIC AAV CAPSID VECTOR ENCODING MODIFIED FACTOR IX POLYPEPTIDES

(71) Applicants: CATALYST BIOSCIENCES, INC., South San Francisco, CA (US); THE BOARD OF TRUSTEES OF THE LELAND STANFORD JUNIOR UNIVERSITY, Stanford, CA (US)

(72) Inventors: Grant E. Blouse, Burlingame, CA (US); Katja Pekrun, Palo Alto, CA (US); Mark A. Kay, Los Altos, CA (US)

(21) Appl. No.: 17/161,602

(22) Filed: Jan. 28, 2021

Related U.S. Application Data
(63) Continuation of application No. PCT/US2020/065431, filed on Dec. 16, 2020.
(60) Provisional application No. 63/045,010, filed on Jun. 26, 2020.
(60) Provisional application No. 62/967,568, filed on Jan. 29, 2020.
(60) Provisional application No. 63/045,010, filed on Jun. 26, 2020.
(60) Provisional application No. 62/967,568, filed on Jan. 29, 2020.

(51) Int. Cl. C07K 14/745; C12N 15/86; A61P 7/04

(52) U.S. Cl. CPC C07K 14/745; C12N 15/86; A61K 38/00; C12N 2750/14143; C12N 2830/42; A61P 7/04

(57) ABSTRACT

AAV vectors that encode a modified Factor IX (FIX) polypeptide for gene therapy for treatment of hemophilia B are provided. The modified FIX polypeptide has increased potency compared to the wild-type FIX polypeptide. The nucleic acid encoding the modified FIX polypeptide includes a portion of an intron. The AAV vectors were generated and selected to infect islet cells, but were found to effectively transduce hepatocytes upon systemic administration, and to express high levels of FIX polypeptide. Relatively low doses of the AAV vectors can be administered to achieve a therapeutic effect. The gene therapy treatment can result in normal or near normal coagulation pharmacokinetics and normal levels of FIX, or mild hemophilia B. Combining an AAV vector with improved properties for transducing hepatocytes, and modified FIX polypeptides with enhanced potency, improves transgene expression and effectively lowers the viral dose needed to achieve therapeutically relevant FIX activity levels.

### RELATED PATENTS AND APPLICATIONS

This application is a continuation of International PCT Application No. PCT/US2020/065431, filed Dec. 16, 2020, entitled “GENE THERAPY FOR HEMOPHILIA B WITH A CHIMERIC AAV CAPSID VECTOR ENCODING MODIFIED FACTOR IX POLYPEPTIDES,” to Applicants Catalyst Biosciences, Inc., and The Board Of Trustees Of The Leland Stanford Junior University, and inventors Grant E. Blouse, Katja Pekrun, and Mark A. Kay.

Benefit of priority is claimed to International PCT Application No. PCT/US2020/065431, which claims benefit of priority, as does this application, to U.S. Provisional Application No. 63/045,010, filed Jun. 26, 2020, entitled “GENE THERAPY FOR HEMOPHILIA B WITH A CHIMERIC AAV CAPSID VECTOR ENCODING MODIFIED FACTOR IX POLYPEPTIDES,” to Applicants Catalyst Biosciences, Inc., and The Board Of Trustees Of The Leland Stanford Junior University, and inventors Grant E. Blouse, Katja Pekrun, and Mark A. Kay.

International PCT Application No. PCT/US2020/065431 also claims benefit of priority, as does this application, to U.S. Provisional Application No. 62/967,568, filed Jan. 29, 2020, entitled “GENE THERAPY FOR HEMOPHILIA B WITH A CHIMERIC AAV CAPSID VECTOR ENCODING MODIFIED FACTOR IX POLYPEPTIDES,” to Applicants Catalyst Biosciences, Inc., and The Board of Trustees of The Leland Stanford Junior University, and inventors Grant E. Blouse, Katja Pekrun, and Mark A. Kay.

This application is related to U.S. Pat. No. 9,328,339, issued May 3, 2016, which is based on U.S. application Ser. No. 14/267,754, filed May 1, 2014, and is related to U.S. Pat. No. 8,778,870, issued Jul. 15, 2014, which is based on U.S. application Ser. No. 13/373,118, filed Nov. 3, 2011, each entitled “MODIFIED FACTOR IX POLYPEPTIDES AND USES THEREOF,” and each to Applicant Catalyst Biosciences, Inc., and inventors Edwin L. Madison, Christopher Thanos, and Grant Ellsworth Blouse.

This application also is related to International PCT Application No. PCT/US2019/025026, filed Mar. 29, 2019, and published as International PCT Application Publication No. WO 2019/191701 on Oct. 3, 2019, and to corresponding U.S. application Ser. No. 16/370,735, filed Mar. 29, 2019, and published as U.S. Patent Application Publication No. US 2020/0024616 A1 on Jan. 23, 2020, each entitled “NOVEL RECOMBINANT ADENO-ASSOCIATED VIRUS CAPSIDS WITH ENHANCED HUMAN PANCREATIC TROPISM,” and each to Applicant The Board of Trustees of The Leland Stanford Junior University, and inventors Katja Pekrun and Mark A. Kay.

The subject matter of each of the above-referenced patents, applications, and publications, including the sequence listings, is incorporated by reference in its entirety.

### INCORPORATION BY REFERENCE OF SEQUENCE LISTING FILED ELECTRONICALLY

An electronic version of the Sequence Listing is filed herewith, the contents of which are incorporated by reference in their entirety. The electronic file was created on Jan. 27, 2020, is 1.603 megabytes in size, and is titled 4956SEQ001.TXT.

### FIELD OF INVENTION

Provided are vectors and gene therapy methods for treatment of hemophilia, particularly hemophilia B. The vectors encode modified FIX polypeptides that have enhanced potency.

### BACKGROUND

Recombinantly produced Factor IX (FIX) polypeptides have been approved for the treatment of hemophilia, in particular, hemophilia B. Also of therapeutic interest are FIX polypeptides that exhibit anticoagulant activities useful in the treatment of thrombolytic diseases. Hence, FIX polypeptides, like other coagulation factors, are important therapeutic agents for procoagulant therapies. While a goal of coagulation therapy is to eliminate bleeds and the adverse consequences of bleeds, it is difficult to achieve. Hence, there is a need for FIX prophylactic therapies and FIX polypeptides for prophylactic use, particularly for effective gene therapy vectors for delivery of FIX polypeptides.

### SUMMARY

Provided are adeno-associated virus (AAV) vectors for gene therapy for the treatment of hemophilia, particularly hemophilia B. The vectors contain chimeric capsids that have increased tropism for the liver compared to wild-type capsids, and contain nucleic acid encoding a modified Factor IX (FIX) polypeptide. The nucleic acid encoding the FIX polypeptide that is encapsulated in the vector includes an intron. The intron can be the first intron or portion thereof from a gene encoding a FIX polypeptide, such as human FIX, such as the human FIX polypeptide set forth in SEQ ID NO: 2 or 325 (mature forms of human FIX are set forth in SEQ ID NOS: 3 and 20, respectively).

The vectors contain nucleic acid constructs. Provided are nucleic acid constructs that contain inverted terminal repeats (ITRs) from an adeno-associated virus, flanking nucleic acid encoding a modified FIX polypeptide, where: the encoded modified FIX polypeptide comprises all or a portion of a FIX gene intron; the encoded modified FIX polypeptide comprises one or more of an insertion, deletion, and replacement of amino acids; and the encoded modified FIX polypeptide, when in activated form, has coagulation activity of at least about 7-10 times the coagulation activity of the activated form of the wild-type FIX polypeptide of SEQ ID NO: 3 or 20. The encoded modified FIX polypeptide that only has the amino acid replacement R338L generally is not a nucleic acid construct provided herein, but a nucleic acid construct that is encapsulated in the chimeric capsids described herein that have increased tropism for the liver.

The chimeric capsids provided herein may comprise sequences from wild-type AAV serotypes. In some examples, the chimeric capsid comprises a mixture of sequences from wild-type AAV serotypes, such as a sequence of two or more AAV serotypes, including AAV1, AAV6, AAV3B, and AAV8. The chimeric capsids transduce hepatocytes with greater efficiency or to a greater extent than any of AAV1, AAV6, AAV3B, and/or AAV8. As a result, the amount of total AAV vector administered to a subject can be lower than the amount of AAV1, AAV6, AAV3B, or AAV8 that would have to be administered to have the same amount of vector or vector genome introduced into the liver, or to obtain a similar therapeutic effect.

Also provided are AAV vectors that contain a capsid designated LK03 of SEQ ID NO:429, or a capsid having at least 95%, 96%, 97%, 98%, or 99% sequence identity therewith; and a nucleic acid construct that encodes a modified FIX polypeptide encapsulated in the capsid, where: the nucleic acid construct comprises inverted terminal repeats (ITRs) from an adeno-associated virus flanking nucleic acid encoding the modified FIX polypeptide; the nucleic acid encoding the modified FIX polypeptide comprises all or a portion of a FIX gene intron; the intron is all or a portion of the first intron of human FIX (hFIX), and the portion is sufficient to increase expression of the encoded FIX polypeptide in a human cell or human to whom the construct is administered for gene therapy; the encoded modified FIX polypeptide comprises one or more of an insertion, deletion, and replacement of amino acids; and the encoded modified FIX polypeptide, when in activated form, has coagulation activity of at least about 7-10 times the activity of the activated form of wild-type FIX of SEQ ID NO:3 or SEQ ID NO:20. In some embodiments, the intron is all or a portion of the first intron of human FIX (hFIX), wherein the portion is sufficient to increase expression of the encoded FIX polypeptide in a human cell or human to whom the construct is administered for gene therapy. For example, the first intron of FIX can comprise the sequence of nucleotides set forth in SEQ ID NO:434, or a sequence having at least 95% or 98% sequence identity therewith. The intron can be inserted after or downstream from nucleic acid encoding the signal sequence, in the nucleic acid encoding the modified FIX polypeptide. For example, the intron is inserted between nucleotides encoding amino acid residues corresponding to residues 29 and 30 of the unmodified FIX polypeptide of SEQ ID NO:2, where corresponding residues are identified by alignment with SEQ ID NO:2 or SEQ ID NO:3.

The AAV vectors contain, for example, a portion of the intron, or comprise at least 10%, at least 12%, at least 15%, at least 16%, or at least 20% of the intron or of the sequence having at least 98% sequence identity therewith, whereby expression of the encoded FIX polypeptide is increased in a human cell or human compared to expression of the FIX polypeptide without the intron. In some embodiments, the intron has the sequence of nucleotides set forth in SEQ ID NO:433, or a sequence that has at least 98% sequence identity therewith. In some embodiments, the intron or portion thereof is inserted between the nucleotides encoding amino acid residues corresponding to residues 29 and 30 of the unmodified FIX polypeptide of SEQ ID NO:2, and corresponding residues are identified by alignment with SEQ ID NO:2 or SEQ ID NO:3.

Among the AAV vectors provided herein, including those with the recombinant adeno-associated viral (rAAV) vectors with LK03, KP-1, KP-2, and KP-3 capsids, are those in which the encoded modified FIX polypeptide, when in activated form, has at least greater than 7-fold, greater than 10-fold, greater than 15-fold, or greater than 20-fold activity, compared to the activity of the activated form of the wild-type FIX of SEQ ID NO: 2, 3, 20, or 325. In the AAV vectors herein, including those with the LK03, KP-1, KP-2, and KP-3 capsids, the construct contains nucleic acid encoding the FIX signal sequence or other suitable heterologous signal sequence, the first residue of the propeptide, the intron, and the remaining residues of the FIX polypeptide, including the remaining residues of the propeptide and mature FIX polypeptide. The AAV vectors herein, including those packaged in the capsids LK03, KP-1, KP-2, and KP-3, encode modified FIX polypeptides, and include a construct, with reference to the unmodified FIX polypeptide of SEQ ID NO:2, that comprises, in the following order: nucleic acid encoding the signal sequence, residue 1 of the propeptide, the intron or portion thereof, residues 2-46 of the propeptide, and residues corresponding to residues 47-461 of the mature FIX polypeptide, where residue positions are determined by alignment with SEQ ID NO:2. Modified FIX includes the modified FIX polypeptide that comprises the sequence of amino acids set forth in SEQ ID NO:394. The modified FIX polypeptide encoded in the AAV vector includes a signal sequence, the intron, and the mature modified FIX polypeptide, as described herein. The modified FIX polypeptides, and the modified FIX polypeptides including the intron and signal sequence, can be encoded by nucleotides optimized for expression in mammals, particularly rodents, such as mice, and/or primates, such as humans. The optimized codons can be modified to also reduce or eliminate CpG islands for expression in primates, such as humans. Exemplary codon-optimized modified FIX polypeptide sequences include those set forth in any of SEQ ID NOs: 562-564 and 569-571, such as the sequence of nucleotides set forth in SEQ ID NO:570 or SEQ ID NO:571. Those of skill in the art can further optimize sequences as appropriate.

Provided are nucleic acid constructs for packaging in the capsids provided herein. The nucleic acid constructs include an intron, such as all or a portion of the first intron of human FIX (hFIX), wherein the portion is sufficient to increase expression of the encoded modified FIX polypeptide in a human cell or human to whom the construct is administered for gene therapy. Exemplary of such introns is the first intron of FIX that comprises the sequence of nucleotides set forth in SEQ ID NO:434, or a sequence having at least 95% or 98% sequence identity therewith. The portion includes at least or at least about 10%, at least 15%, at least 16%, or at least 20% of the intron or of the sequence having at least 95% or 98% sequence identity therewith, whereby expression of the encoded modified FIX polypeptide is increased in a human cell or human compared to expression of the FIX polypeptide without the intron. An exemplary partial intron is one having the sequence of nucleotides set forth in SEQ ID NO:433, or a sequence having at least 98% sequence identity therewith. The intron can consist of the sequence of nucleotides set forth in SEQ ID NO:433. The intron can be inserted at a site such that the expression of the encoded modified FIX polypeptide is increased compared to expression in the absence of any intron. For example, the intron can be inserted between nucleic acids encoding amino acid residues corresponding to residues 29 and 30 of the unmodified FIX polypeptide of SEQ ID NO:2, where corresponding residues are identified by alignment with SEQ ID NO:2 or SEQ ID NO:3.

The nucleic acid constructs for packaging in the capsids described herein can include any encoding a modified FIX polypeptide that, when in activated form, has at least greater than 7-fold, greater than 10-fold, greater than 15-fold, or greater than 20-fold activity, compared to the activity of the activated form of the wild-type FIX of SEQ ID NO: 2, 3, 20 or 325. An exemplary construct is one that contains nucleic acid encoding the FIX signal sequence, the first residue of the propeptide, the intron, the remaining residues of the FIX polypeptide, including the remaining residues of the propeptide and mature FIX, such as a construct, with reference (for alignment) to the unmodified FIX of SEQ ID NO:2, that includes the signal sequence (residues 1-28), residue 1 of the propeptide, the intron, residues 2-46 of the propeptide, and residues 47-461 of mature FIX, wherein residue positions are determined by alignment with SEQ ID NO:2. Exemplary of the modified FIX polypeptides that have improved properties (increased activity and/or potency) is the polypeptide whose sequence of the mature form is set forth in SEQ ID NO:394 (or the same sequence in which the residue at T148 is T148A (SEQ ID NO:486)). Among the constructs provided herein are those that comprise the sequences of nucleotides set forth in any of SEQ ID NOS: 562-564 and 569-571, and further optimized forms thereof, such as by elimination or reduction of CpG islands, which can increase expression in primates, such as humans.

For the modified FIX polypeptides described herein, the codons (in upper case letters) encoding the mutations were introduced with the following primers (it is understood that the skilled person can substitute other degenerate codons, including any that are optimized for expression in a human):

| | | SEQ |
|---|---|---|
| Primer Name | Primer Sequence (5′ to 3′) | ID NO. |
| F9-A[103]N/ | gtaaaaatagtAATgatAGCaaggtggtttg | 28 |
| N[105]S-For | | |
| F9-D[104]N/ | gtaaaaatagtgctAATaacAGTgtggtttgctoctgtactg | 29 |
| K[106]S-For | | |
| F9-K[106]N/ | gtgctgataacAATgtgAGTtgctoctgtactg | 30 |
| V[108]S-For | | |
| F9-D[85]N-For | gaactgtgaattaAATgtaacatgtaac | 31 |
| F9-T[148]A-For | ctcacccgtgctgagGCTgtttttcctgatgtg | 32 |
| F9-D39N/F41T-For | gaatggtaaagttAATgcaACCtgtggaggctctatc | 33 |
| F9-K63N-For | gaaactggtgttAACattacagttgtcgc | 34 |
| F9-I86S-For | gcgaaatgtgAGTcgaattattcctc | 35 |
| F9-A95bS-For | caactacaatgcaAGTattaataagtacaac | 36 |
| F9-K243N-For | aaggaaaaaacaAATctcacttaagtgctagctg | 37 |
| F9-E240N-For | ctggattaagAATaaaacaaagctc | 38 |
| F9-E74N-For | caggtgaacataatattAACgagacagaacatacag | 39 |
| F9-T76N/H78S-For | gaacataatattgaggagAACgaaAGTacagagcaaaag | 40 |
| F9-K82N/N845-For | cagaacatacagagcaaAATcgaTCTgtgattcgaattattc | 41 |
| F9-L153N-For | gggagatcagctAATgttcttcagtac | 42 |
| F9-F145N/H1475- | ctggggaagagtcAACTCCaaagggagatcag | 43 |
| For | | |
| F9-K222N/K2245- | gagtgtgcaatgAACggcTCAtatggaatatatac | 44 |
| For | | |
| F9-S151N/L1535- | cttccacaaagggagaAATgctTCAgttcttca | 45 |
| For | | |
| F9-N95S-For | cctcaccacaactacAGTgcagctattaataagtacaacc | 46 |
| F9-Y117N-For | cttagtgctaaacagcAACgttacacctatttgc | 47 |
| F9-G149N-For | ggaagagtcttccacaaaAACagatcagctttagttc | 48 |
| F9-R150N/A152S- | gtcttccacaaagggAACtcaTCTttagttcttcagtac | 49 |
| For | | |
| F9-R150A-For | gtcttccacaaagggGCAtcagctttagttcttcag | 50 |
| F9-R150E-For | gtcttccacaaagggGAAtcagctttagttcttcag | 51 |
| F9-R150Y-For | gtcttccacaaagggTACtcagctttagttcttcag | 52 |
| F9-R143Q-For | gtaagtggctggggaCAAgtcttccacaaaggg | 53 |
| F9-R143A-For | gtaagtggctggggaGCAgtcttccacaaaggg | 54 |
| F9-R143Y-For | gtaagtggctggggaTACgtcttccacaaaggg | 55 |
| F9-R143L-For | gtaagtggctggggaCTGgtcttccacaaaggg | 56 |
| F9-V38M-For | gttttgaatggtaaaATGgatgcattctgtggaggc | 57 |
| F9-V38Y-For | gttttgaatggtaaaTACgatgcattctgtggaggc | 58 |
| F9-D39M-For | gttttgaatggtaaagttATGgcattctgtggaggc | 59 |
| F9-D39Y-For | gttttgaatggtaaagttTACgcattctgtggaggc | 60 |
| F9-A40M-For | gttttgaatggtaaagttgatATGttctgtggaggctctatc | 61 |
| F9-A40Y-For | gttttgaatggtaaagttgatTACttctgtggaggctctatc | 62 |
| F9-R233A/K230A- | caaatatggaatatataccGCAgtatccGCAtatgtcaactgg | 63 |
| For | attaag | |
| F9-R233E/K230E- | caaatatggaatatataccGAAgtatccGAAtatgtcaactgg | 64 |
| For | attaag | |
| F9-R233A-For | gaatatataccaaggtatccGCAtatgtcaactggattaag | 65 |
| F9-R233E-For | gaatatataccaaggtatccGAAtatgtcaactggattaag | 66 |
| F9-K230A-For | caaatatggaatatataccGCAgtatcccggtatgtc | 67 |
| F9-K230E-For | caaatatggaatatataccGAAgtatcccggtatgtc | 68 |
| F9-K126E-For | cctatttgcattgctgacGAAgaatacacgaacatc | 69 |
| F9-K126A-For | cctatttgcattgctgacGCAgaatacacgaacatc | 70 |
| F9-R165A-For | gttccacttgttgacGCAgccacatgtcttcgatct | 71 |
| F9-R165E-For | gttccacttgttgacGAAgccacatgtcttcgatct | 72 |
| F9-R170A-For | cgagccacatgtcttGCAtctacaaagttcacc | 73 |
| F9-R170E-For | cgagccacatgtcttGAAtctacaaagttcacc | 74 |
| F9-D[64]N-For | ggcggcagttgcaagAACgacattaattcctatG | 273 |
| F9-D[64]A-For | ggcggcagttgcaagGCTgacattaattcctatG | 274 |
| F9-N[157]Q-For | cctgatgtggactatgtaCAGtctactgaagctgaaacc | 275 |
| F9-N[157]D-For | cctgatgtggactatgtaGACtctactgaagctgaaacc | 276 |
| F9-N[167]Q-For | gaaaccattttggatCAGatcactcaaagcacc | 277 |
| F9-N[167]D-For | gaaaccattttggatGACatcactcaaagcacc | 278 |
| F9-S[61]A-For | ccatgtttaaatggcggcGCTtgcaaggatgacattaattcc | 279 |
| F9-S[53]A-For | gatggagatcagtgtgagGCTaatccatgtttaaatggc | 280 |
| F9-T[159]A-For | gtggactatgtaaattctGCTgaagctgaaaccattttg | 281 |
| F9-T[169]A-For | CattttggataacatcGCTcaaagcacccaatcatttaatgac | 282 |
| F9-T[172]A-For | gataacatcactcaaagcGCTcaatcatttaatgac | 283 |
| F9-T[179]A-For | caatcatttaatgacttcGCTcgggttgttggtggagaaG | 284 |
| F9-Y[155]F-For | gtttttcctgatgtggacTTCgtaaattctactgaagctG | 285 |
| F9-Y[155]H-For | gtttttcctgatgtggacCACgtaaattctactgaagctG | 286 |
| F9-Y[155]Q-For | gtttttcctgatgtggacCAGgtaaattctactgaagctG | 287 |
| F9-S[158]A-For | gtggactatgtaaatGCTactgaagctgaaacc | 288 |
| F9-S[158]D-For | gtggactatgtaaatGACactgaagctgaaacc | 289 |
| F9-S[158]E-For | gtggactatgtaaatGAGactgaagctgaaacc | 290 |
| F9-R165S-For | gttccacttgttgacAGCgccacatgtcttcgatct | 291 |
| F9-R170L-For | cgagccacatgtcttCTGtctacaaagttcacc | 292 |
| F9-K148N-For | ggaagagtcttccacAACgggagatcagctttaG | 293 |
| F9-K148A-For | ggaagagtcttccacGCTgggagatcagctttaG | 294 |
| F9-K148E-For | ggaagagtcttccacGAGgggagatcagctttaG | 295 |
| F9-K148S-For | ggaagagtcttccacAGCgggagatcagctttaG | 296 |
| F9-K148M-For | ggaagagtcttccacATGgggagatcagctttaG | 297 |
| F9-E745-For | ggtgaacataatattAGCgagacagaacatacaG | 298 |
| F9-E74A-For | ggtgaacataatattGCTgagacagaacatacaG | 299 |
| F9-E74R-For | ggtgaacataatattAGGgagacagaacatacaG | 300 |
| F9-E74K-For | ggtgaacataatattAAGgagacagaacatacaG | 301 |
| F9-H92F-For-Corr | cgaattattcctcacTTCaactacaatgcaGC | 302 |
| F9-H92Y-For-Corr | cgaattattcctcacTACaactacaatgcaGC | 303 |
| F9-H92E-For-Corr | cgaattattcctcacGAAaactacaatgcaGC | 304 |
| F9-H92S-For-Corr | cgaattattcctcacAGCaactacaatgcaGC | 305 |
| F9-T242A-For | CtggattaaggaaaaaGCTaagctcacttaagtg | 306 |
| F9-T242V-For | CtggattaaggaaaaaGTGaagctcacttaagtg | 307 |
| F9-E240N/T242A- | gtcaactggattaagAACaaaGCTaagctcacttaagtg | 308 |
| For | | |
| F9-E240N/T242V- | gtcaactggattaagAACaaaGTGaagctcacttaagtg | 309 |
| For | | |
| F9-E240Q-For | gtcaactggattaagCAGaaaacaaagctcacttaaG | 310 |
| F9-E240S-For | gtcaactggattaagAGCaaaacaaagctcacttaaG | 311 |
| F9-E240A-For | gtcaactggattaagGCTaaaacaaagctcacttaaG | 312 |
| F9-E240D-For | gtcaactggattaagGACaaaacaaagctcacttaaG | 313 |
| F9-N178D-For | CAaagttcaccatctatGACaacatgttctgtgctggc | 314 |
| F9-N178Y-For | CAaagttcaccatctatTACaacatgttctgtgctggc | 315 |
| F9-Y177A-For | CTacaaagttcaccatcGCTaacaacatgttctgtGC | 316 |
| F9-Y177T-For | CTacaaagttcaccatcACCaacaacatgttctgtGC | 317 |
| F9-T175R-For | cttcgatctacaaagttcAGGatctataacaacatgttc | 318 |
| F9-T175E-For | cttcgatctacaaagttcGAAatctataacaacatgttc | 319 |
| F9-T175Q-For | cttcgatctacaaagttcCAGatctataacaacatgttc | 320 |
| F9-F174I-For | GTcttcgatctacaaagATCaccatctataacaacatg | 321 |
| F9-T175R/Y177T- | cgatctacaaagttcAGGatcACCaacaacatgttctgtG | 322 |
| For | | |
| F9-Y94F/K98T-For | GAattattcctcaccacaacTTCaatgcagctattaatACCta | 323 |
| | caaccatgacattG | |
| F9-F145N/K148S-For | ggctggggaagagtcAACcacAGCgggagatcagctttaG | 324 |

In one example, the construct encodes a modified FIX polypeptide that includes an amino acid replacement T343R, T343E, or T343D, or the same replacement at a corresponding amino acid residue in an unmodified FIX polypeptide; an amino acid replacement at amino acid residue R318 or at a residue corresponding to 318, wherein the amino acid replacement is selected from among Y, E, F, and W; and/or an amino acid replacement R338E or R338D; where the unmodified FIX polypeptide comprises the sequence of amino acids set forth in SEQ ID NO: 2, 3, 20, or 325; and residue positions are referenced by mature numbering, and identified by alignment with SEQ ID NO:3. For example, the modified FIX polypeptide comprises replacements corresponding to R338E/T343R; and the unmodified FIX polypeptide comprises the sequence of amino acids set forth in SEQ ID NO: 2, 3, 20, or 325. The modified FIX polypeptide can further include a replacement corresponding to R318Y, whereby the resulting encoded modified FIX polypeptide comprises replacements corresponding to R318Y/R338E/T343R. Exemplary of an encoded mature modified FIX polypeptide is one that comprises the sequence of amino acids set forth in SEQ ID NO:394, or SEQ ID NO:394 in which residue 148 is A (alanine), or a sequence having at least 95% sequence identity therewith, which contains the replacements corresponding to R318Y/R338E/T343R. Other exemplary encoded modified FIX polypeptides include those that comprise replacements corresponding to replacements R318Y/R338E, or R318Y/T343R, or R318Y/E410N, or R338L, where the unmodified FIX polypeptide comprises the sequence of amino acids set forth in SEQ ID NO: 2, 3, 20, or 325. Modified FIX polypeptides can include those that comprise an amino acid replacement R318Y, and an amino acid replacement at an amino acid residue selected from among residues 338, 343, 403, and 410 of a mature FIX polypeptide having a sequence set forth in SEQ ID NO:3, or at amino acid residues corresponding to residues 338, 343, 403, or 410 in an unmodified FIX polypeptide, such as a modified FIX polypeptide comprising an amino acid replacement selected from among R338E, T343R, R403E, and E410N in a mature FIX polypeptide having a sequence set forth in SEQ ID NO:3, or the same replacements at corresponding amino acid residues in an unmodified FIX polypeptide. Exemplary modified FIX polypetides with replacements, in unmodified FIX polypeptides comprising the sequence of amino acids set forth in SEQ ID NO: 2, 3, 20, or 325, or other allelic variants, include modified FIX polypeptides with amino acid replacements selected from among replacements: R318Y/R338E/R403E/E410N, R318Y/R338E/T343R/R403E/E410N, R318Y/R338E/T343R/E410N, Y155F/R318Y/R338E/T343R/R403E, Y155F/K228N/K247N/N249S/R318Y/R338E/T343R/R403E/E410N, Y155F/K247N/N249S/R318Y/R338E/T343R/R403E, R318Y/R338E/T412A, K247N/N249S/R318Y/R338E/T343R/R403E, R318Y/R338E/T343R, Y155F/K247N/N249S/R318Y/R338E/T343R, K228N/R318Y/R338E/T343R/R403E/E410N, K228N/K247N/N249S/R318Y/R338E/T343R/R403E, R318Y/R338E/T343R/R403E/E410S, Y155F/K247N/N249S/R318Y/R338E, K247N/N249S/R318Y/R338E/T343R, R318Y/T343R/E410N, Y155F/R318Y/R338E/R403E, R318Y/R338E/R403E, R318Y/R338E/E410N, K228N/R318Y/E410N, R318Y/R403E/E410N, D203N/F205T/R318Y/E410N, A103N/N105S/R318Y/R338E/R403E/E410N, D104N/K106S/R318Y/R338E/R403E/E410N, K228N/R318Y/R338E/R403E/E410N, I125S/R318Y/R338E/R403E/E410N, D104N/K106S/I251S/R318Y/R338E/R403E/E410N, D104N/K106S/R318Y/R338E/E410N, I251S/R318Y/E410N/R338E, D104N/K106S/I251S/R318Y/R338E/E410N, A103N/N105S/K247N/N249S/R318Y/R338E/R403E/E410N, D104N/K106S/K247N/N249S/R318Y/R338E/R403E/E410N, K228N/K247N/N249S/R318Y/R338E/R403E/E410N, A103N/N105S/Y155F/R318Y/R338E/R403E/E410N, D104N/K106S/Y155F/R318Y/R338E/R403E/E410N, Y155F/K228N/R318Y/R338E/R403E/E410N, Y155F/I251S/R318Y/R338E/R403E/E410N, K247N/N249S/R318Y/R338E/R403E/E410N, Y155F/R318Y/R338E/R403E/E410N, K247N/N249S/R318Y/R338E/E410N, Y155F/R318Y/R338E/E410N, Y155F/K247N/N249S/R318Y/R338E/E410N, R318Y/R338E/R403E/E410S, R318Y/R338E/R403E/E410N/T412V, R318Y/R338E/R403E/E410N/T412A, R318Y/R338E/R403E/T412A, R318Y/R338E/E410S, R318Y/R338E/T412A, R318Y/R338E/E410N/T412V, D85N/K228N/R318Y/R338E/R403E/E410N, N260S/R318Y/R338E/R403E/E410N, R318Y/R338E/N346D/R403E/E410N, Y155F/R318Y/R338E/N346D/R403E/E410N, K247N/N249S/N260S/R318Y/R338E/R403E/E410N, D104N/K106S/N260S/R318Y/R338E/R403E/E410N, Y155F/N260S/R318Y/R338E/R403E/E410N, Y155F/R318Y/R338E/T343R/R403E/E410N, D104N/K106S/Y155F/K247N/N249S/R318Y/R338E/R403E/E410N, D104N/K106S/K228N/K247N/N249S/R318Y/R338E/R403E/E410N, Y155F/K228N/K247N/N249S/R318Y/R338E/R403E/E410N, Y155F/K247N/N249S/N260S/R318Y/R338E/R403E/E410N, D104N/K106S/R318Y/R338E/T343R/R403E/E410N, R318Y/R338E/N346Y/R403E/E410N, R318Y/R338E/T343R/N346Y/R403E/E410N, R318Y/R338E/T343R/N346D/R403E/E410N, R318Y/R338E/Y345A/R403E/E410N, K228N/I251S/R318Y/R338E/R403E/E410N, R318Y/R338E/Y345A/N346D/R403E/E410N, Y155F/K247N/N249S/R318Y/R338E/R403E, K247N/N249S/R318Y/R338E/R403E, Y155F/K247N/N249S/R318Y/R403E/E410N, K247N/N249S/R318Y/R403E/E410N, R318Y/R338E/T343R/R403E, Y155F/R318Y/R338E/T343R/E410N, R318Y/T343R/R403E/E410N, Y155F/R318Y/T343R/R403E/E410N, Y155F/K247N/N249S/R318Y/R338E/T343R/R403E/E410N, K247N/N249S/R318Y/R338E/T343R/R403E/E410N, Y155F/K228N/I251S/R318Y/R338E/R403E/E410N, N260S/R318Y/R338E/T343R/R403E/E410N, Y155F/N260S/R318Y/R338E/T343R/R403E/E410N, K228N/K247N/N249S/R318Y/R338E/T343R/R403E/E410N, Y155F/K247N/N249S/R318Y/R403E, Y155F/K247N/N249S/R318Y/E410N, K247N/N249S/R318Y/R338E/T343R/E410N, Y155F/K247N/N249S/R318Y/R338E/T343R/E410N, Y155F/K247N/N249S/R318Y/T343R/R403E/E410N, K247N/N249S/R318Y/T343R/R403E/E410N, Y155F/K247N/N249S/R318Y/T343R/R403E, K247N/N249S/R318Y/T343R/R403E, Y155F/K247N/N249S/R318Y/T343R/E410N, K247N/N249S/R318Y/T343R/E410N, Y155F/R318Y/R338E/T343R, Y155F/R318Y/T343R/R403E, Y155F/R318Y/T343R/E410N, K228N/K247N/N249S/R318Y/R338E/T343R/E410N, Y155F/K247N/N249S/R318Y/T343R, Y155F/K247N/N249S/R318Y/R338E/R403E/E410N, K228N/K247N/N249S/R318Y/T343R/R403E/E410N, and Y155F/R318Y/R403E/E410N, where numbering is with respect to the mature FIX polypeptide of SEQ ID NO:3. Other such exemplary modified FIX polypeptides include those that comprise amino acid replacements selected from among: R318Y/R338E/R403E/E410N, R318Y/R338E/T343R/R403E/E410N, R318Y/R338E/T343R/E410N, Y155F/R318Y/R338E/T343R/R403E, Y155F/K228N/K247N/N249S/R318Y/R338E/T343R/R403E/E410N, Y155F/K247N/N249S/R318Y/R338E/T343R/R403E, K247N/N249S/R318Y/R338E/T343R/R403E, R318Y/R338E/T343R, Y155F/K247N/N249S/R318Y/R338E/T343R, K228N/R318Y/R338E/T343R/R403E/E410N, K228N/K247N/N249S/R318Y/R338E/T343R/R403E, R318Y/R338E/T343R/R403E/E410S, Y155F/K247N/N249S/R318Y/R338E, K247N/N249S/R318Y/R338E/T343R, R318Y/T343R/E410N, and Y155F/R318Y/R338E/R403E; where numbering is with respect to the mature FIX polypeptide of SEQ ID NO:3; and the unmodified FIX polypeptide comprises the sequence of amino acids set forth in SEQ ID NO: 2, 3, 20, or 325. Particular embodiments include those in which the modified FIX polypeptide comprises the amino acid replacements R318Y/R338E/R403E/E410N, R318Y/R338E/T343R/R403E/E410N, or R318Y/R338E/T343R/E410N.

Nucleic acid encoding the modified FIX polypeptides include those in which the mature portion of FIX, corresponding to residues 1-415 of SEQ ID NO:3, is encoded by the sequence of nucleotides set forth in any of SEQ ID NOs:483-487, such as the sequence of nucleotides set forth in SEQ ID NO: 483 or 486. The portion encoding the modified FIX polypeptide and the intron inserted in the nucleic acid encoding FIX, such as between residues 428 and 429, comprises the sequence of nucleotides set forth in any of SEQ ID NOs:462-467. The codons can be optimized for expression in a human cell. It is found herein that codons optimized for expression in mice are very similar, such as at least about 90% similar, to human optimized sequences. Codon optimized sequences include those set forth in SEQ ID NOs:518-521, where SEQ ID NOs:518-521 are as follows:

| | Encoded Residues | |
|---|---|---|
| SEQ ID | of Mature Fix | |
| NO. | (SEQ ID NO: 2) | Description |
| 518 | encodes 47-461 | hWT-FIX mature codon optimized for human |
| | | 0.5 optimization; |
| 519 | encodes 47-461 | hWT-FIX-mature codon optimized for |
| | | human 0.8 optimization; |
| 520 | encodes 47-461 | hWT-FIX-mature codon optimized for |
| | | human 1.0 optimization. |

Provided are nucleic acid constructs in which the nucleic acid encoding the mature modified FIX polypeptides, corresponding to residues 47-461 of SEQ ID NO: 2, and residues 1-415 of SEQ ID NO: 3, has the sequence of nucleotides set forth in any of SEQ ID NOs:521-526, wherein SEQ ID NOs:521-526 are as follows:

| | Encoded Residues | |
|---|---|---|
| SEQ ID | of Mature Fix | |
| NO. | (SEQ ID NO: 2) | Description |
| 521 | encodes 47-461 | human FIX R318Y/R338E/T343R -FIX |
| | | mature codon optimized for human 0.5 |
| | | optimization; |
| 522 | encodes 47-461 | human FIX R318Y/R338E/T343R -FIX |
| | | mature codon optimized for human 0.8 |
| | | optimization; |
| 523 | encodes 47-461 | human FIX R318Y/R338E/T343R -FIX |
| | | mature codon optimized for human 1.0 |
| | | optimization; |
| 524 | encodes 47-461 | human FIX R338L-FIX mature codon |
| | | optimized for human 0.5 optimization; |
| 525 | encodes 47-461 | human FIX R338L-FIX mature codon |
| | | optimized for human 0.8 optimization; and |
| 526 | encodes 47-461 | human FIX R338L-FIX mature codon |
| | | optimized for human 1.0 optimization. |

Other optimized nucleic acid sequences include those encoding residues 30-461 of SEQ ID NO:2, having the sequence of nucleotides set forth in any of SEQ ID NOs:527-529, where SEQ ID NOs:527-529 are as follows:

| | Encoded Residues | |
|---|---|---|
| SEQ ID | of Mature Fix | |
| NO. | (SEQ ID NO: 2) | Description |
| 527 | encodes 30-461 | hWT-FIX codon optimized for human 0.5 |
| | | optimization; |
| 528 | encodes 30-461 | hWT-FIX codon optimized for human 0.8 |
| | | optimization; and |
| 529 | encodes 30-461 | hWT-FIX codon optimized for human 1.0 |
| | | optimization. |

Others are nucleic acid encoding the modified FIX polypeptide, corresponding to residues 30-461 of SEQ ID NO:2, that comprise the sequence of nucleotides set forth in any of SEQ ID NOs:530-535, where SEQ ID NOs:530-535 are as follows:

| | Encoded Residues | |
|---|---|---|
| SEQ ID | of Mature Fix | |
| NO. | (SEQ ID NO: 2) | Description |
| 530 | encodes 30-461 | human FIX R318Y/R338E/T343R -FIX codon |
| | | optimized for human 0.5 optimization; |
| 531 | encodes 30-461 | human FIX R318Y/R338E/T343R -FIX codon |
| | | optimized for human 0.8 optimization; |
| 532 | encodes 30-461 | human FIX R318Y/R338E/T343R -FIX codon |
| | | optimized for human 1.0 optimization; |
| 533 | encodes 30-461 | human FIX R338L-FIX codon optimized for |
| | | human 0.5 optimization; |
| 534 | encodes 30-461 | human FIX R338L-FIX codon optimized for |
| | | human 0.8 optimization; and |
| 535 | encodes 30-461 | human FIX R338L-FIX codon optimized for |
| | | human 1.0 optimization. |

Other exemplary nucleic acid constructs with optimized codons include those that include the signal sequence, the partial intron, and wild-type or modified human FIX, and comprise the sequence of nucleotides set forth in any of SEQ ID NOs: 536-553, where SEQ ID NOs: 536-553 are as follows:

| SEQ ID | Sequence | |
|---|---|---|
| NO. | Includes | Description |
| 536 | precursor + intron | hFIXss-Pro-mini Intron-Pro-human wild type |
| | | FIX optimized 0.5; |
| 537 | precursor + intron | hFIXss-Pro-mini Intron-Pro-human wild type |
| | | FIX optimized 0.8; |
| 538 | precursor + intron | hFIXss-Pro-mini Intron-Pro-human wild type |
| | | FIX optimized 1.0; |
| 539 | precursor + intron | hFIXss-Pro-mini Intron-Pro-human FIX |
| | | R318Y/R338E/T343R optimized 0.5; |
| 540 | precursor + intron | hFIXss-Pro-mini Intron-Pro-human FIX |
| | | R318Y/R338E/T343R optimized 0.8; |
| 541 | precursor + intron | hFIXss-Pro-mini Intron-Pro-human FIX |
| | | R318Y/R338E/T343R optimized 1.0; |
| 542 | precursor + intron | hFIXss-Pro-mini Intron-Pro-human FIX |
| | | R338L optimized 0.5; |
| 543 | precursor + intron | hFIXss-Pro-mini Intron-Pro-human FIX |
| | | R338L optimized 0.8; |
| 544 | precursor + intron | hFIXss-Pro-mini Intron-Pro-human FIX |
| | | R338L optimized 1.0; |
| 545 | precursor + intron | mouse optimized hFIXss-Pro-mini Intron-Pro- |
| | | human wild type FIX optimized 0.5; |
| 546 | precursor + intron | mouse optimized hFIXss-Pro-mini Intron-Pro- |
| | | human wild type FIX optimized 0.8; |
| 547 | precursor + intron | mouse optimized hFIXss-Pro-mini Intron-Pro- |
| | | human wild type FIX optimized 1.0; |
| 548 | precursor + intron | mouse optimized hFIXss-Pro-mini Intron-Pro- |
| | | human FIX R318Y/R338E/T343R optimized 0.5; |
| 549 | precursor + intron | mouse optimized hFIXss-Pro-mini Intron-Pro- |
| | | human FIX R318Y/R338E/T343R optimized 0.8; |
| 550 | precursor + intron | mouse optimized hFIXss-Pro-mini Intron-Pro- |
| | | human FIX R318Y/R338E/T343R optimized 1.0; |
| 551 | precursor + intron | mouse optimized hFIXss-Pro-mini Intron-Pro- |
| | | human FIX R338L optimized 0.5; |
| 552 | precursor + intron | mouse optimized hFIXss-Pro-mini Intron-Pro- |
| | | human FIX R338L optimized 0.8; and |
| 553 | precursor + intron | mouse optimized hFIXss-Pro-mini Intron-Pro- |
| | | human FIX R338L optimized 1.0. |

The sequences can be optimized, and regulatory regions can be selected for expression in a hepatocyte (in the liver).

Exemplary of constructs provided is one in which the portion encoding the modified FIX polypeptide and the intron comprises the sequence of nucleotides set forth in SEQ ID NO:466. Others include a construct comprising 2 ITRs (inverted terminal repeats) flanking the nucleic acid comprising nucleic acid encoding the FIX polypeptide with the intron, wherein the ITR is an AAV ITR, or a chimeric or hybrid AAV ITR. The AAV can be from any serotype, such as serotypes 1-11, and hybrids and chimeras thereof. Exemplary of an ITR is an AAV2 ITR. Exemplary ITR sequences include those that comprises the sequence of nucleotides set forth in SEQ ID NO: 435 or 437, or the ITRs set forth as residues 1-119 and 4281-4410 of SEQ ID NO:456, or an ITR of any of SEQ ID NOs: 436 and 501-511.

The constructs include transcription regulatory sequences operatively linked to the nucleic acid for transcription of the nucleic acid encoding the modified FIX polypeptide. In particular, they include liver-specific regulatory sequences, such as a promoter operatively linked for expression of the nucleic acid encoding the modified FIX polypeptide. In general, the selected promoter is a liver-specific or hepatocyte-specific promoter, such as, but not limited to, the human alpha-1 antitrypsin promoter (hAAT) (also called serpin A1 anti-trypsin promoter), or the hybrid liver-specific promoter (HLP), or a transthyretin (TTR) promoter. Exemplary promoter sequences are set forth in SEQ ID NOs:440 and 441.

The constructs also can include a transcription factor, particularly a transcription factor that is liver-specific, such as an enhancer. Exemplary enhancers are an ApoE/C1 gene locus enhancer, or the serpin A1 liver enhancer; exemplary sequences are set forth in SEQ ID NO:438 or SEQ ID NO:439, respectively. The constructs include transcription terminators, such as polyA sequences, for transcription termination, and others, such as the bGHpolyA terminator whose sequence is set forth in SEQ ID NO:443. In some examples, the polyA sequence is included for enhanced expression of the modified FIX polypeptide.

Exemplary constructs include those comprising the sequence of nucleotides set forth in any of SEQ ID NOs:456-461, such as the construct comprising the sequence of nucleotides set forth in SEQ ID NO:460. Also provided are vectors for producing the constructs. These include any of the vectors of any of SEQ ID NOs:447-455.

Provided are AAV vectors (also referred to as AAV virions) that comprise a capsid, and any of the constructs encoding a modified FIX polypeptide encapsulated therein. The AAV vector is one that transduces human hepatocytes with greater transduction efficiency, or with a reduced amount of AAV vector introduced, compared to the AAV capsid designated DJ/8, or can be one that transduces human and mouse hepatocytes with at least or substantially the same efficiency, or to the same extent, as the AAV capsid designated DJ/8. The vector can be one where the capsid is a chimeric capsid, such as a chimeric capsid comprising wild-type AAV serotypes. The chimeric capsid can comprise a mixture of sequences from two or more AAV serotypes, for example, two or more of AAV1, AAV6, AAV3B, and AAV8. The AAV vector can be one that also transduces islet cells. Exemplary of AAV vectors are those in which the capsid comprises the sequence of amino acids set forth in any of SEQ ID NOs: 418-420, and 492-500, or a sequence having at least 95% sequence identity therewith. These include AAV vectors with a capsid designated KP-1, KP-2 or KP-3, such as the capsid designated KP-1, whose sequence of amino acids is set forth in SEQ ID NO:418. Others include those encoded by the sequence of nucleotides set forth in any of SEQ ID NOs: 421-423, or a sequence having at least 95% sequence identity therewith. The encoded modified FIX polypeptide includes any described herein, or known to those of skill in the art, to have enhanced potency. These include the modified FIX polypeptide comprising the sequence of amino acids set forth in SEQ ID NO:394, or SEQ ID NO:394 in which residue 148 is A (alanine; SEQ ID NO:486). The construct in the vector can be one that has the sequence set forth in SEQ ID NO:460, or in SEQ ID NO:461.

Also provided are capsids, such as the capsid set forth in SEQ ID NO:561, for encapsulating a modified FIX polypeptide provided herein, where the provided AAV vectors comprise a capsid and any of the constructs encoding a modified FIX polypeptide encapsulated therein. Also provided are pharmaceutical compositions that contain the AAV vector comprising the capsid of SEQ ID NO:561. Also provided are methods of treating hemophilia, wherein the pharmaceutical composition containing the AAV vector comprising the capsid of SEQ ID NO:561 is administered to a subject with hemophilia.

Also provided are pharmaceutical compositions that contain any of the AAV vectors provided and described herein in a pharmaceutically acceptable vehicle. The vectors include the AAV vector that encodes a modified FIX polypeptide comprising the sequence of amino acids set forth in SEQ ID NO:394, or in SEQ ID NO:394 in which residue 148 is A (alanine; SEQ ID NO:486).

The pharmaceutical compositions can be formulated for single dosage administration, where a single dosage is about 10⁸ to 10¹⁵ viral genomes (vg), or 10⁹ to 10¹³ genome copies (gc) per dose, assuming an average human has a mass of about 75 kg. Other single dosages include a single dosage of about or at 10¹⁰ to 10¹³ vg or gc, or 10⁸ to 10¹¹ vg or gc, or 10⁸ to 10¹² vg or gc, or 10⁹ to 10¹¹ vg or gc, or 10⁸ to 10¹¹ vg or gc, assuming an average human has a mass of about 75 kg. The pharmaceutical compositions also can be formulated for single dosage administration, where a single dosage is about 10⁸ to 10¹⁶ viral genomes (vg), or 10⁹ to 10¹⁴ genome copies (gc) per dose, assuming an average human has a mass of about 75 kg. The pharmaceutical compositions can be formulated for any desired route of administration, including, but not limited to, parenteral, systemic, intravenous, intramuscular, oral, rectal, subcutaneous, direct injection into the liver, and other such routes. Direct injection into the liver can be effected by compartmentalizing the liver (such as by clamping liver), so that it is isolated from systemic circulation during and following injection of the vector or virus into the parenchyma of the liver (see, e.g., U.S. Pat. No. 9,821,114). Compartmentalization is maintained for at least 15, at least 20, at least 25, or at least 30 minutes, up to an hour, following injection into the liver. As a result, the vector or virus is quantitatively taken up by the liver parenchyma, so that there is little or no systemic exposure to the vector or virus. This eliminates adverse effects, such as viremia and immune reactions, and permits lower doses to be administered.

Provided are methods of treating hemophilia or uses of the provided vectors and pharmaceutical compositions for treatment of hemophilia. Treatment is effected by administering the AAV vectors or the pharmaceutical compositions provided herein to a subject who has hemophilia. Hemophilias include hemophilia B, and hemophilia B with inhibitors. The treatment should be one that results in at least 20%, at least 30%, at least 40%, or at least 50% normal clotting activity, so that the subject has mild hemophilia, or normal clotting, or reduced annualized bleeds. The subject is one who has a hemophilia, particularly hemophilia B. Exemplary of the encoded modified FIX polypeptide is the modified FIX polypeptide that comprises the sequence of amino acids set forth in SEQ ID NO:394, or in SEQ ID NO:394 in which residue 148 is A (alanine; SEQ ID NO:486). Administration can be effected by any suitable route, including parenterally, systemically, intra-muscularly, rectally, orally, or by direct injection into the liver. Exemplary of the modified FIX polypeptide is the modified FIX polypeptide that comprises the sequence of amino acids set forth in SEQ ID NO:394, or in SEQ ID NO:394 in which residue 148 is A (alanine; SEQ ID NO:486).

For the uses and methods, the amount of vector is any that is therapeutically effective, which can be determined by the skilled artisan, and depends upon the subject, the severity of disease, and other such parameters. Exemplary dosages are 10⁶ to 10¹³ vg/kg or gc/kg, such as 10⁸ to 10¹¹ vg/kg of the subject or gc/kg of the subject. The vector or pharmaceutical composition is administered intravenously. An exemplary dosage is 10⁶ to 10¹⁰ vg/kg or gc/kg of the subject. Lower dosages can be administered when the vector or pharmaceutical composition is administered via direct injection into the liver.

Methods for producing the AAV vector encoding the modified FIX polypeptide include packaging any of the constructs into a capsid that has the desired properties, including the transduction of the liver. Such capsids include those comprising the sequence of amino acids set forth in any of SEQ ID NOs: 418-420 and 492-500. The encoded modified FIX polypeptide is any that has the requisite activity/potency. Exemplary of the encoded modified FIX polypeptide is the modified FIX polypeptide that comprises the sequence of amino acids set forth in SEQ ID NO:394, or in SEQ ID NO:394 in which residue 148 is A (alanine; SEQ ID NO:486).

Modified FIX polypeptides for use in the AAV vector as described herein are exemplary of FIX polypeptides for use for gene therapy using the vectors and methods described herein. Encoding nucleic acids are encapsulated in the capsids described herein, such as the capsids with the sequences set forth in SEQ ID NOs: 418-420. The modified FIX polypeptides have improved procoagulant therapeutic properties compared to unmodified FIX polypeptide (recombinant FIX, such as BeneFIX® FIX, see, SEQ ID NOs: 20 and 325, and also, compared to the modified extended half-life forms). For example, among the modified FIX polypeptides for use in the vectors and methods herein are modified FIX polypeptides that exhibit increased coagulant activity, increased catalytic activity, increased resistance to AT-III, increased resistance to heparin and/or the AT-III/heparin complex, and/or improved pharmacokinetic properties, such as i) decreased clearance, ii) altered (e.g., increased or decreased) volume of distribution, iii) enhanced in vivo recovery, iv) enhanced total protein exposure in vivo (i.e., AUC), v) increased serum half-life (α-, β-, and/or γ-phase), and/or vi) increased mean resonance time (MRT). The higher potency and bioavailability and longer half-life and other properties permit a sufficiently low dose that is suitable for gene therapy, so that with the longer half-life, a steady state level of FIX is achieved.

Exemplary of modified FIX polypeptides for encoding in the AAV vectors as described herein are the modified FIX polypeptides that comprises replacements corresponding to R338E/T343R, where the unmodified FIX polypeptide comprises the sequence of amino acids set forth in SEQ ID NO: 3 or 20. The modified FIX polypeptide can include the replacement corresponding to R318Y alone, or in combination with replacements corresponding to R338E/T343R.

The modified FIX polypeptides encoded in the rAAV vectors provided herein can have an amino acid replacement at residue R318 or at a residue corresponding to 318, wherein the amino acid replacement is selected from among Y, E, F, and W; and/or an amino acid replacement T343R, T343E, or T343D, or the same replacement at a corresponding amino acid residue in an unmodified FIX polypeptide; and/or an amino acid replacement at amino acid position Y155, or at a residue corresponding to 155, that is selected from F or L.

The modified FIX polypeptide can comprise an amino acid replacement at residue R338 or at a residue corresponding to R338 in an unmodified FIX polypeptide; an amino acid replacement at residue T343 or at an amino acid residue corresponding to amino acid residue T343 in an unmodified FIX polypeptide; and/or an amino acid replacement at residue E410 or at an amino acid residue corresponding to amino acid residue E410 in an unmodified FIX polypeptide; and/or an amino acid replacement at an amino acid residue selected from among D203, F205, and K228, or at an amino acid residue corresponding to amino acid residue D203, F205, or K228 in an unmodified FIX polypeptide. The replacement at residue R339 can be D, E, or L. Combinations include R318Y/R338E, or the same replacements at corresponding amino acid residues in an unmodified FIX polypeptide. The modified FIX polypeptide can comprise an amino acid replacement T343R or T343K, which can be combined with a replacement at residue R318, such as the replacements R318Y/T343R or the same replacements at corresponding amino acid residues in an unmodified FIX polypeptide. The modified FIX polypeptide can further include an amino acid replacement at residue E410, or at an amino acid residue corresponding to 410 in an unmodified FIX polypeptide, that is N or S. The modified FIX polypeptide can comprise amino acid replacements R318Y/E410N, or the same replacements at corresponding amino acid residues in an unmodified FIX polypeptide. The modified FIX polypeptide can comprise an amino acid replacement R318Y and an amino acid replacement at an amino acid residue selected from among residues 338, 343, 403, and 410 of a mature FIX polypeptide having a sequence set forth in SEQ ID NO:3, or at amino acid residues corresponding to residues 338, 343, 403, or 410 in an unmodified FIX polypeptide. These include modified FIX polypeptides comprising an amino acid replacement selected from among R338E or R338L, T343R, R403E and E410N, in a mature FIX polypeptide having a sequence set forth in SEQ ID NO:3 or the same replacements at corresponding amino acid residues in an unmodified FIX polypeptide. Exemplary modified FIX polypeptides comprise the replacements T343R/Y345T, T343R/N346D, T343R/N346Y, R338E/T343R, R338E/T343R/R403E/E410N, R338E/T343R/R403E, R338E/T343R/R403E/E410S, N260S/R338E/T343R/R403E, R338E/T343R/R403E/E410N, R338E/T343R/E410N, R338E/R403E/E410N, Y155F/R338E/T343R/R403E/E410N, Y155F/R338E/T343R/R403E, Y155F/R338E/T343R/R403E/E410S, Y155F/N260S/R338E/T343R/R403E, Y155F/T343R/R403E/E410N, Y155F/R338E/T343R/E410N, and Y155F/R338E/R403E/E410N. The modified FIX polypeptide additionally can include a replacement at the residue corresponding to R318.

Provided herein are methods and regimens for the prophylactic treatment of hemophilia B by administering subcutaneously modified FIX polypeptides containing an amino acid replacement in an unmodified FIX polypeptide, where the amino acid replacement can be one or more of replacement of tyrosine (Y) at amino acid residue R318 (R318Y), R318E, R318F, R318W, R318D, R3181, R318K, R318L, R318M, R318S, R318V, S61A, S61C, S61D, S61E, S61F, S61G, S61I, S61K, S61L, S61P, S61R, S61V, S61W, S61Y, D64A, D64C, D64F, D64H, D641, D64L, D64M, D64P, D64R, D64S, D64T, D64W, Y155F, Y155L, N157D, N157E, N157F, N157I, N157K, N157L, N157M, N157R, N157V, N157W, N157Y, S158A, S158D, S158E, S158F, S158G, S158I, S158K, S158L, S158M, S158R, S158V, S158W, S158Y, N167D, N167Q, N167E, N167F, N167G, N167H, N167I, N167K, N167L, N167M, N167P, N167R, N167V, N167W, N167Y, T169A, T169D, T169E, T169F, T169G, T169I, T169K, T169L, T169M, T169P, T169R, T169S, T169V, T169W, T169Y, T172A, T172D, T172E, T172F, T172G, T172I, T172K, T172L, T172M, T172P, T172R, T172S, T172V, T172W, T172Y, D203M, D203Y, D203F, D203H, D203I, D203K, D203L, D203R, D203V, D203W, A204M, A204Y, A204F, A204I, A204W, E239S, E239R, E239K, E239D, E239F, E2391, E239L, E239M, E239T, E239V, E239W, E239Y, H257F, H257E, H257D, H2571, H257K, H257L, H257M, H257Q, H257R, H257V, H257W, R312Y, R312L, R312C, R312D, R312E, R312F, R312I, R312K, R312M, R312P, R312S, R312T, R312V, R312W, K316M, K316D, K316F, K316H, K316I, K316L, K316R, K316V, K316W, K316Y, F342I, F342D, F342E, F342K, F342L, F342M, F342S, F342T, F342V, F342W, F342Y, T343R, T343E, T343D, T343F, T343I, T343K, T343L, T343M, T343S, T343V, T343W, T343Y, N346Y, N346E, N346F, N346H, N346I, N346K, N346L, N346M, N346Q, N346R, N346V, N346W, K400E, K400C, K400D, K400F, K400G, K400L, K400M, K400P, K400S, K400T, K400V, K400Y, R403D, R403F, R4031, R403K, R403L, R403M, R403S, R403V, R403Y, E410D, E410S, E410A, E410F, E410G, E410I, E410K, E410L, E410M, E410P, E410R, E410T, E410V, E410W, E410Y, T412A, T412V, T412C, T412D, T412E, T412F, T412G, T412I, T412M, T412P, T412W, or T412Y, in a mature FIX polypeptide having a sequence set forth in SEQ ID NO:3, or the same replacement at a corresponding amino acid residue in an unmodified FIX polypeptide, wherein corresponding amino acid residues are identified by alignment of the unmodified FIX polypeptide with the polypeptide of SEQ ID NO:3; and provided that the modified FIX polypeptide does not contain the modifications F342I/T343R/Y345T. In particular, provided herein are prophylactic subcutaneous methods and regimens in which the modified FIX polypeptides contain the amino acid replacements R318Y/R338E/R403E/E410N, R318Y/R338E/T343R/R403E/E410N, R318Y/R338E/T343R/E410N, Y155F/R318Y/R338E/T343R/R403E, Y155F/K228N/K247N/N249S/R318Y/R338E/T343R/R403E/E410N, Y155F/K247N/N249S/R318Y/R338E/T343R/R403E, K247N/N249S/R318Y/R338E/T343R/R403E, R318Y/R338E/T343R, Y155F/K247N/N249S/R318Y/R338E/T343R, K228N/R318Y/R338E/T343R/R403E/E410N, K228N/K247N/N249S/R318Y/R338E/T343R/R403E, R318Y/R338E/T343R/R403E/E410S, Y155F/K247N/N249S/R318Y/R338E, K247N/N249S/R318Y/R338E/T343R, R318Y/T343R/E410N, Y155F/R318Y/R338E/R403E, Y155F/R338E/T343R/R403E/E410N, Y155F/K247N/N249S/R338E/R403E/E410N, K247N/N249S/R338E/T343R/R403E/E410N, or R338E/T343R/E410N. In some embodiments, the FIX polypeptide comprises the replacement R338L in place of the replacement R338E, or contains the replacement R338L in addition to other replacements.

The modified FIX polypeptide for use in the prophylactic subcutaneous methods and regimens can contain two amino acid replacements in an unmodified FIX polypeptide, where: the first amino acid replacement is at an amino acid residue selected from among residues at positions 53, 61, 64, 85, 103, 104, 105, 106, 108, 155, 158, 159, 167, 169, 172, 179, 202, 203, 204, 205, 228, 239, 241, 243, 247, 249, 251, 257, 259, 260, 262, 265, 284, 293, 312, 314, 315, 316, 317, 318, 319, 321, 333, 338, 343, 345, 346, 392, 394, 400, 403, 410, 412, and 413, in a mature FIX polypeptide having a sequence set forth in SEQ ID NO:3; or a corresponding amino acid residue in an unmodified FIX polypeptide, wherein corresponding amino acid residues are identified by alignment of the unmodified FIX polypeptide with the FIX polypeptide of SEQ ID NO:3; and the second amino acid replacement is at an amino acid residue selected from among residues at positions 5, 53, 61, 64, 85, 155, 158, 159, 167, 239, 260, 284, 293, 312, 318, 333, 338, 346, 400, 403, 410, 412, and 413, in a mature FIX polypeptide having a sequence set forth in SEQ ID NO:3, or a corresponding amino acid residue in an unmodified FIX polypeptide, wherein corresponding amino acid residues are identified by alignment of the unmodified FIX polypeptide with the FIX polypeptide of SEQ ID NO:3.

The first amino acid replacement in the modified FIX polypeptide can be selected from among S53A, S61A, D64A, D64N, D85N, A103N, D104N, N105S, K106S, K106N, V108S, Y155F, Y155H, Y155Q, S158A, S158D, S158E, T159A, N167D, N167Q, T169A, T172A, T179A, V202M, V202Y, D203M, D203Y, A204M, A204Y, K228N, E239A, E239N, E239S, E239R, E239K, T241N, H243S, K247N, N249S, I251S, H257F, H257E, H257F, H257Y, H257S, Y259S, N260S, A262S, K265T, Y284N, K293E, K293A, R312Q, R312A, R312Y, R312L, F314N, H315S, K316S, K316N, K316A, K316E, K316S, K316M, G317N, R318A, R318E, R318Y, R318N, S319N, A320S, L321S, K321N, R333A, R333E, R333S, R338A, R338E, R338L, F342I, T343R, T343E, T343Q, Y345A, Y345T, N346D, N346Y, K392N, K394S, K400A, K400E, R403A, R403E, E410Q, E410N, E410D, E410S, E410A, T412A, T412V, and K413N, or a conservative amino acid replacement, or the same replacement at a corresponding amino acid residue in an unmodified FIX polypeptide; and the second amino acid replacement is selected from among K5A, S53A, S61A, D64A, D64N, D85N, Y155F, Y155H, Y155Q, S158A, S158D, S158E, T159A, N167D, N167Q, E239A, E239N, E239S, E239R, E239K, N260S, Y284N, K293E, K293A, R312Q, R312A, R312Y, R312L, R318A, R318E, R318Y, R318N, R333A, R333E, R333S, R338A, R338E, R338L, N346D, N346Y, K400A, K400E, R403A, R403E, E410Q, E410N, E410D, E410S, E410A, T412A, T412V, and K413N, or a conservative amino acid replacement, or the same replacement at a corresponding amino acid residue in an unmodified FIX polypeptide. For example, the first amino acid replacement is at a position selected from among 318, 155, 247, 249, 338, 403, and 410, or at a corresponding amino acid residue in an unmodified FIX polypeptide; and the second amino acid replacement is at a position selected from among 338, 155, 247, 249, 318, 403, and 410, or is at a corresponding amino acid residue in an unmodified FIX polypeptide. Exemplary of these are embodiments where the first amino acid replacement is selected from among R318Y, Y155F, K247N, N249S, R338E, R403E, and E410N, or is the same amino acid replacement at a corresponding amino acid residue in an unmodified FIX polypeptide; and the second amino acid replacement is selected from among R338E, Y155F, K247N, N249S, R318Y, R403E, and E410N, or is the same replacement at a corresponding amino acid residue in an unmodified FIX polypeptide. The polypeptides can include these replacements, and, additionally or alternatively, amino acid replacements selected from among amino acid replacements K400E/R403E, D85N/K228N, D85N/I251S, K400A/R403A, R338A/R403A, R338E/R403E, K293A/R403A, K293E/R403E, R318A/R403A, R338E/E410N, K228N/E410N, K228N/R338E, K228N/R338A, and R403E/E410N, or the same replacements at corresponding amino acid residues in an unmodified FIX polypeptide.

In some examples, the first or the second amino acid replacement is replacement with an amino acid residue selected from among alanine (Ala, A); arginine (Arg, R); asparagine (Asn, N); aspartic acid (Asp, D); cysteine (Cys, C); glutamic acid (Glu, E); glutamine (Gln, Q); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V), providing that the replacing amino acid is not the same as the amino acid it replaces. In particular examples, the first amino acid replacement is replacement with an amino acid residue selected from among alanine; asparagine; aspartic acid, glutamic acid; glutamine; histidine; isoleucine; leucine; lysine; methionine; phenylalanine; serine; threonine; tyrosine; and valine. For example, exemplary amino acid replacements include S53A, S61A, D64A, D64N, D85N, A103N, D104N, N105S, K106S, K106N, V108S, Y155F, Y155H, Y155Q, S158A, S158D, S158E, T159A, N167D, N167Q, T169A, T172A, T179A, V202M, V202Y, D203M, D203Y, A204M, A204Y, K228N, E239A, E239N, E239S, E239R, E239K, T241N, H243S, K247N, N249S, I251S, H257F, H257E, H257F, H257Y, H257S, Y259S, N260S, A262S, K265T, Y284N, K293E, K293A, R312Q, R312A, R312Y, R312L, F314N, H315S, K316S, K316N, K316A, K316E, K316S, K316M, G317N, R318A, R318E, R318Y, R318N, S319N, A320S, L321S, R333A, R333E, R333S, R338A, R338E, R338L, T343R, T343E, T343Q, F342I, Y345A, Y345T, N346D, N346Y, K392N, K394S, K400A, K400E, R403A, R403E, E410Q, E410N, E410D, E410S, E410A, T412A, T412V, or K413N. Other exemplary amino acid replacements are conservative amino acid replacements thereof.

In some instances, the second amino acid replacement is replacement with an amino acid residue selected from among alanine; arginine; asparagine; aspartic acid; glutamic acid; glutamine; histidine; leucine; lysine; phenylalanine; serine; threonine; tyrosine; or valine. For example, exemplary amino acid replacements include K5A, S53A, S61A, D64A, D64N, D85N, Y155F, Y155H, Y155Q, S158A, S158D, S158E, T159A, N167D, N167Q, E239A, E239N, E239S, E239R, E239K, N260S, Y284N, K293E, K293A, R312Q, R312A, R312Y, R312L, R318A, R318E, R318Y, R318N, R333A, R333E, R333S, R338A, R338E, R338L, N346D, N346Y, K400A, K400E, R403A, R403E, E410Q, E410N, E410D, E410S, E410A, T412A, T412V, or K413N. Other exemplary amino acid replacements are conservative amino acid replacements thereof.

In particular examples, the first amino acid replacement is at a position corresponding to a position selected from among 155, 247, 249, 318, 338, 403, and 410, such as, for example, Y155F, K247N, N249S, R318Y, R338E, R403E, and E410N. In further examples, the second amino acid replacement is at a position corresponding to a position selected from among 155, 247, 249, 318, 338, 403, and 410, such as, for example, Y155F, K247N, N249S, R318Y, R338E, R403E, and E410N.

Among the modified FIX polypeptides for use in the methods and regimens provided herein are those containing amino acid replacements selected from among amino acid replacements corresponding to K400E/R403E, R318E/R403E, R318Y/E410N, K228N/R318Y, Y155F/K228N, Y155F/I251S, Y155F/N346D, Y155F/N260S, R338E/T343R, E410N/T412A, E410N/T412V, R318Y/R338E, D85N/K228N, D85N/I251S, K400A/R403A, R338A/R403A, R338E/R403E, K293A/R403A, K293E/R403E, R318A/R403A, R338E/E410N, K228N/E410N, K228N/R338E, K228N/R338A, and R403E/E410N.

In some examples, the modified FIX polypeptides contain one or more further amino acid replacements, such as one or more replacements at a position selected from among 53, 61, 64, 85, 103, 104, 105, 106, 108, 155, 158, 159, 167, 169, 172, 179, 202, 203, 204, 205, 228, 239, 241, 243, 247, 249, 251, 257, 259, 260, 262, 265, 284, 293, 312, 314, 315, 316, 317, 318, 319, 321, 333, 338, 343, 346, 345, 392, 394, 400, 403, 410, 412, and 413, in a mature FIX polypeptide having a sequence set forth in SEQ ID NO:3. For example, the modified FIX polypeptides can contain a further amino acid replacement selected from among Y5A, S53A, S61A, D64A, D64N, D85N, A103N, D104N, N105S, K106S, K106N, V108S, Y155F, Y155H, Y155Q, S158A, S158D, S158E, T159A, N167D, N167Q, T169A, T172A, T179A, V202M, V202Y, D203M, D203Y, A204M, A204Y, K228N, E239A, E239N, E239S, E239R, E239K, T241N, H243S, K247N, N249S, I251S, H257F, H257E, H257F, H257Y, H257S, Y259S, N260S, A262S, K265T, Y284N, K293E, K293A, R312Q, R312A, R312Y, R312L, F314N, H315S, K316S, K316N, K316A, K316E, K316S, K316M, G317N, R318A, R318E, R318Y, R318N, S319N, A320S, L321S, R333A, R333E, R333S, R338A, R338E, R338L, T343R, T343E, T343Q, F342I, Y345A, Y345T, N346D, N346Y, K392N, K394S, K400A, K400E, R403A, R403E, E410Q, E410N, E410D, E410S, E410A, T412A, T412V, and K413N, or a conservative amino acid replacement thereof.

In some examples, the modified FIX polypeptides for use in the methods provided herein contain amino acid replacements selected from among amino acid replacements corresponding to R318Y/R338E/R403E, D203N/F205T/R318Y, R318Y/R338E/E410N, K228N/R318Y/E410N, R318Y/R403E/E410N, R318Y/R338E/T412A, R318Y/R338E/R403E/E410N, D203N/F205T/R318Y/E410N, A103N/N105S/R318Y/R338E/R403E/E410N, D104N/K106S/R318Y/R338E/R403E/E410N, K228N/R318Y/R338E/R403E/E410N, I125S/R318Y/R338E/R403E/E410N, D104N/K106S/I251S/R318Y/R338E/R403E/E410N, D104N/K106S/R318Y/E410N/R338E, I125S/R318Y/E410N/R338E, D104N/K106S/I251S/R318Y/E410N/R338E, A103N/N105S/Y155F, D104N/K106S/Y155F, Y155F/K247N/N249S, A103N/N105S/K247N/N249S/R318Y/R338E/R403E/E410N, D104N/K106S/K247N/N249S/R318Y/R338E/R403E/E410N, K228N/K247N/N249S/R318Y/R338E/R403E/E410N, A103N/N105S/Y155F/R318Y/R338E/R403E/E410N, D104N/K106S/Y155F/R318Y/R338E/R403E/E410N, Y155F/K228N/R318Y/R338E/R403E/E410N, Y155F/I251S/R318Y/R338E/R403E/E410N, Y155F/K247N/N249S/R318Y/R338E/R403E/E410N, K247N/N249S/R318Y/R338E/R403E/E410N, Y155F/R318Y/R338E/R403E/E410N, K247N/N249S/R318Y/R338E/E240N, Y155F/R318Y/R338E/E410N, Y155F/K247N/N249S/R318Y/R338E/E410N, D104N/K106S/Y155F/K228N/K247N/N249S, D104N/K106S/Y155F/K247N/N249S, D104N/K106S/Y155F/K228N, Y155F/K228N/K247N/N249S, R318Y/R338E/R403E/E410S, R318Y/R338E/R403E/E410N/T412V, R318Y/R338E/R403E/E410N/T412A, R318Y/R338E/R403E/T412A, R318Y/R338E/E410S, R318Y/R338E/T412A, R318Y/R338E/E410N/T412V, D85N/K228N/R318Y/R338E/R403E/E410N, N260S/R318Y/R338E/R403E/E410N, R318Y/R338E/N346D/R403E/E410N, Y155F/R318Y/R338E/N346D/R403E/E410N, Y155F/N260S/N346D, K247N/N249S/N260S/R318Y/R338E/R403E/E410N, D104N/K106S/N260S/R318Y/R338E/R403E/E410N, Y155F/N260S/R318Y/R338E/R403E/E410N, R318Y/R338E/T343R/R403E/E410N, D104N/K106S/Y155F/N260S, Y155F/K247N/N249S/N260S, D104N/K106S/Y155F/K247N/N249S/N260S, D104N/K106S/Y155F/K228N, D104N/K106S/Y155F/K247N/N249S, D85N/D203N/F205T, D85N/D104N/K106S/I251S, K293A/R338A/R403A, K293E/R338E/R403E, R338E/R403E/E410N, D203N/F205T/K228N, D203N/F205T/E410N, D203N/F205T/R338E, D203N/F205T/R338A, D203N/F205T/R338E/R403E, K228N/R338E/R403E, K247N/N249S/N260S, D104N/K106S/N260S, K228N/K247N/N249S/D104N/K106S, A103N/N105S/K228N, D104N/K106S/K228N, A103N/N105S/I251S, D104N/K106S/I251S, A103N/N105S/K247N/N249S, D104N/K106S/K247N/N249S, K228N/K247N/N249 S, D104N/K106S/K228N/K247N/N249S, K247N/N249S/N260S, D104N/K106S/N260S, Y259F/K265T/Y345T, and D104N/K106S/K247N/N249S/N260S.

Also provided for use in the methods herein are modified FIX polypeptides containing a modification in an unmodified FIX polypeptide, wherein the modification is selected from among modifications corresponding to amino acid replacements S61A, D64A, Y155F, N157D, S158A, S158D, S158E, N167D, N167Q, T169A, T172A, D203M, D203Y, A204M, A204Y, E239S, E239R, E239K, H257F, H257E, R312Y, R312L, K316M, R318E, R318Y, T343R, T343E, F342I, N346Y, K400E, E410D, E410S, E410A, T412A, and T412V, in a mature FIX polypeptide having a sequence set forth in SEQ ID NO:3. In some examples, the modified FIX polypeptide contains two or more of the amino acid replacements.

In particular instances, the modified FIX polypeptide contains the mutation Y155F. For example, provided are modified FIX polypeptides that contain the replacement Y155F, and a modification at an amino acid position selected from among positions corresponding to 247, 249, 338, 403, and 410, of a mature FIX polypeptide having a sequence set forth in SEQ ID NO:3. In one example, the modified FIX polypeptide contains the replacements Y155F/K247N/N249S. In further instances, the modified FIX polypeptide contains the mutation R318Y. For example, provided are modified FIX polypeptides containing the replacement R318Y and a modification at an amino acid position selected from positions corresponding to 338, 403, and 410 of a mature FIX polypeptide having a sequence set forth in SEQ ID NO:3, such as, for example, R338E, R403E, or E410N.

In some examples, the modified FIX polypeptides contain one or more further modifications at an amino acid position selected from among positions corresponding to 5, 53, 61, 64, 85, 103, 104, 105, 106, 108, 148, 155, 157, 158, 159, 167, 169, 172, 179, 202, 202, 203, 204, 205, 228, 239, 241, 243, 247, 249, 251, 257, 259, 260, 262, 265, 284, 293, 312, 314, 315, 316, 317, 318, 319, 320, 321, 333, 338, 343, 345, 346, 392, 394, 400, 403, 410, 412, and 413, of a mature FIX polypeptide having a sequence set forth in SEQ ID NO:3. Exemplary modification(s) are selected from among modifications corresponding to amino acid replacements K5A, S53A, S61A, D64A, D64N, D85N, A103N, D104N, N105S, N105T, K106N, K106N, K106T, V108S, V108T, T148A, Y155F, Y155H, N157D, N157Q, S158A, S158D, S158E, T159A, N167D, N167Q, T169A, T172A, T179A, V202M, V202Y, D203M, D203Y, D203N, A204M, A204Y, F205S, F205T, K228N, E239N, T241N, E239S, E239A, E239R, E239K, H243S, H243T, K247N, N249S, N249T, I251S, I251T, H257F, H257Y, H257E, H257S, N260S, A262S, A262T, Y284N, K293E, K293A, R312Q, R312A, R312Y, R312L, F314N, H315S, K316S, K316T, K316M, G317N, R318E, R318Y, R318N, R318A, S319N, A320S, L321N, L321S, L321T, R333A, R333E, R338A, R338E, T343R, T343E, T343Q, F342I, Y345A, Y345T, N346D, N346T, K392N, K394S, K394T, K400A, K400E, R403A, R403E, E410Q, E410S, E410N, E410A, E410D, T412V, T412A, and K413N.

Thus, provided herein are methods and regimens for subcutaneous prophylaxis of hemophilia B that include the administration of modified FIX polypeptides containing modifications selected from among modifications corresponding to amino acid replacements K400E/R403E, R318E/R403E, R318Y/E410N, R318Y/R338E/R403E, D203N/F205T/R318Y, K228N/R318Y, R318Y/R338E/E410N, K228N/R318Y/E410N, R318Y/R403E/E410N, R318Y/R338E/R403E/E410N, D203N/F205T/R318Y/E410N, A103N/N105S/R318Y/R338E/R403E/E410N, D104N/K106S/R318Y/R338E/R403E/E410N, K228N/R318Y/R338E/R403E/E410N, I251S/R318Y/R338E/R403E/E410N, D104N/K106S/I251S/R318Y/R338E/R403E/E410N, D104N/K106S/R318Y/E410N/R338E, I251S/R318Y/E410N/R338E, D104N/K106S/I251S/R318Y/E410N/R338E, A103N/N105S/Y155F, D104N/K106S/Y155F, Y155F/K228N, Y155F/I251S, Y155F/K247N/N249S, A103N/N105S/K247N/N249S/R318Y/R338E/R403E/E410N, D104N/K106S/K247N/N249S/R318Y/R338E/R403E/E410N, K228N/K247N/N249S/R318Y/R338E/R403E/E410N, A103N/N105S/Y155F/R318Y/R338E/R403E/E410N, D104N/K106S/Y155F/R318Y/R338E/R403E/E410N, Y155F/K228N/R318Y/R338E/R403E/E410N, Y155F/I251S/R318Y/R338E/R403E/E410N, Y155F/K247N/N249S/R318Y/R338E/R403E/E410N, K247N/N249S/R318Y/R338E/R403E/E410N, Y155F/R318Y/R338E/R403E/E410N, K247N/N249S/R318Y/R338E/E240N, Y155F/R318Y/R338E/E410N, Y155F/K247N/N249S/R318Y/R338E/E410N, D104N/K106S/Y155F/K228N/K247N/N249S, D104N/K106S/Y155F/K247N/N249S, D104N/K106S/Y155F/K228N, Y155F/K228N/K247N/N249S, R318Y/R338E/R403E/E410S, R318Y/R338E/R403E/E410N/T412V, R318Y/R338E/R403E/E410N/T412A, R318Y/R338E/R403E/T412A, R318Y/R338E/E410S, R318Y/R338E/T412A, R318Y/R338E/E410N/T412V, D85N/K228N/R318Y/R338E/R403E/E410N, N260S/R318Y/R338E/R403E/E410N, R318Y/R338E/N346D/R403E/E410N, Y155F/N346D, Y155F/R318Y/R338E/N346D/R403E/E410N, Y155F/N260S, Y155F/N260S/N346D, K247N/N249S/N260S/R318Y/R338E/R403E/E410N, D104N/K106S/N260S/R318Y/R338E/R403E/E410N, Y155F/N260S/R318Y/R338E/R403E/E410N, R318Y/R338E/T343R/R403E/E410N, D104N/K106S/Y155F/N260S, Y155F/K247N/N249S/N260S, R338E/T343R, D104N/K106S/Y155F/K247N/N249S/N260S, D104N/K106S/Y155F/K228N, D104N/K106S/Y155F/K247N/N249S, T343R/Y345T, E410N/T412A, R410N/T412V, and R318Y/R338E. In particular examples, the modified FIX polypeptides contain modifications corresponding to the amino acid replacements R318Y/R338E/R403E/E410N, or Y155F/K247N/N249S/R318Y/R338E/R403E/E410N.

In some instances, the unmodified FIX polypeptide contains a sequence of amino acids set forth in any of SEQ ID NOs: 2, 3, 20, or 325, or is a species variant thereof, or a variant having at least 60% sequence identity with the FIX polypeptide of any of SEQ ID NOs: 2, 3, 20, or 325, or is an active fragment of a FIX polypeptide that comprises a sequence of amino acids set forth in any SEQ ID NOs: 2, 3, 20, or 325. For example, the species variant can have the sequence of amino acids set forth in any of SEQ ID NOs: 4-18. In other examples, the variant having at least 60% sequence identity with the FIX polypeptide of any of SEQ ID NOs: 2, 3, 20, or 325, has a sequence of amino acids set forth in any of SEQ ID NOs: 75-272. In further examples, the modified FIX polypeptide is an active fragment of an unmodified FIX polypeptide; and the modified FIX polypeptide contains the modification(s) described herein.

Any of the modified FIX polypeptides for use in the methods and regimens provided herein can contain one or more modifications that introduces and/or eliminates one or more glycosylation sites compared to the unmodified FIX polypeptide. In some examples, the glycosylation sites are selected from among, N-, O-, and S-glycosylation sites. In one example, one or more N-glycosylation sites are introduced compared to the unmodified FIX polypeptide. In some examples, the N-glycosylation site is introduced at amino acid positions corresponding to positions selected from among Y1, S3, G4, K5, L6, E7, F9, V10, Q11, G12, L14, E15, R16, M19, E20, K22, S24, F25, E26, E27, A28, R29, E30, V31, F32, E33, T35, E36, R37, T39, E40, F41, W42, K43, Q44, Y45, V46, D47, G48, D49, Q50, E52, S53, N54, L57, N58, G59, S61, K63, D65, 166, N67, S68, Y69, E70, W72, P74, F77, G79, K80, N81, E83, L84, D85, V86, T87, N89, 190, K91, N92, R94, K100, N101, 5102, A103, D104, N105, K106, V108, 5110, E113, G114, R116, E119, N120, Q121, K122, S123, E125, P126, V128, P129, F130, R134, V135, S136, S138, Q139, T140, S141, K142, A146, E147, A148, V149, F150, P151, D152, V153, D154, Y155, V156, S158, T159, E160, A161, E162, T163, I164, L165, D166, I168, T169, Q170, S171, T172, Q173, S174, F175, N176, D177, F178, T179, R180, G183, E185, D186, K188, P189, K201, V202, D203, E213, E224, T225, G226, K228, E239, E240, T241, H243, K247, N249, I251, R252, I253, P255, H257, N258, N260, A261, A262, I263, N264, K265, A266, D276, E277, P278, V280, N282, S283, Y284, D292, K293, E294, N297, I298, K301, F302, G303, S304, Y306, R312, F314, H315, K316, G317, R318, S319, L321, V322, Y325, R327, P329, L330, D332, R333, A334, T335, L337, R338, K341, F342, T343, Y345, N346, H354, E355, G357, R358, Q362, E372, E374, G375, E388, M391, K392, G393, K394, R403, N406, K409, E410, K411, and K413, of the mature FIX polypeptide set forth in SEQ ID NO:3.

Exemplary modifications that introduce a glycosylation site include those selected from among modifications corresponding to amino acid replacements Y1N, Y1N+S3T, S3N+K5S/T, G4T, G4N+L6S/T, K5N+E7T, L6N+EBT, E7N+F9T, F9N+Q11S/T, V10N+G12S/T, Q11N+N13T, G12N+L14S/T, L14N+R16T, E15T, E15N+E17T; R16N+C18S/T, M19N+E21T; E20N+K22T, K22N, S24N+E26T; F25N+E27T; E26N+A28T; E27N+R29T; A28N+E30T; R29N+V31S/T, E30N+F32T; V31N+E33T; F32N+N34T, E33N, T35N+R37S/T, E36T; E36N; R37N, T39N+F41S/T, E40N+W42T, F41N+K43S/T, W42N+Q44S/T, K43N+Y45T; Q44N+V46S/T, Y45N+D47T, V46N+G48S/T, D47N+D49S/T, G48N+Q50S/T, D49N+C51S/T, Q50N+E52S/T, E52N+N54T, S53N+P55S/T, C56S/T, L57N+G59S/T, G59N+S61T; G60S/T, S61N+K63S/T, K63N+D65S/T, D65N+N67S/T, I66N+S68S/T, Y69S/T, Y69N+C71S/T, S68N+E70S/T, E70N+W72S/T, W72N+P74S/T, P74N+G76S/T, F75N, G76N+E78T, E78N+K80T, F77T, F77N+G79S/T, G79N+N81S/T, K80N+C82S/T, E83S/T, E83N+D85S/T, L84N+V86S/T, D85N, V86A, V86N+C88S/T, T87N+N89S/T, I90N+N92S/T, K91S/T, I90N+N92S/T, K91N+G93 S/T, R94S/T, R94N+E96S/T, K100N, A103S/T, S102N+D104S/T, A103N+N105S/T, D104N+K106S/T, V107S/T, K106N+V108S/T, V108N+V110S/T, S111N, E113N+Y115S/T, G114N+R116S/T, R116N+A118S/T, E119N+Q121S/T, K122S/T, Q121N+S123S/T, K122N+C124S/T S123N+E125S/T, E125N+A125S/T, P126N+V128S/T, A127N+P129T, V128N+F130S/T, P129N+P131S/T, F130N+C132S/T, R134N, V135N+V137S/T, S136N, S138N, V137N+Q139T; Q139N, T140N+L142S/T, S141N+L143S/T, K142N, A146N+A148S/T, E147N+V149S/T, T148N+F150S/T, V149N+P151S/T, F150N+D152S/T, P151N+V153S/T, D152N+D154S/T, V153N+Y155S/T, D154N+V156S/T, Y155N+N157S/T, V156N, S158N+E160S/T, T159N+A161S/T, E160N+E162S/T, A161N, E162N+I164S/T, T163N+L165S/T, I164N+D166S/T, L165N+N167S/T, D166N+I168S/T, I168N+Q170S/T, T169N, Q170N, S171N+Q173S/T, T172N, Q173N+F175S/T, S174N+N176S/T, F175N+D177S/T, F178S/T, D177N, D177E, F178N+R180S/T, T179N+V181S/T, R180N+V182S/T, G183+E185S/T, G184N+D186T, E185N+A187S/T, D186N+K188S/T, A187N+P189T, K188N+G190S/T, P189N+Q181S/T, G200N+V202T, K201N+D203S/T, K201T, V202N+A204S/T, D203N+F205S/T, E213N+W215S/T, K214T, V223T, E224N+G226S/T, T225N+V227S/T, G226N+K228S/T, V227N+I229T, K228N, H236N+I238T; I238N+E240T; E239N, E240N+E242S/T, E242N, T241N+H243S/T, H243N+E245S/T, K247N+N249S/T, V250N+R252T, I251S/T, I251N+I253S/T, R252N+I254S/T, I253N+P255S/T, P255N+H257S/T, H257N+Y259S/T, N260S/T, A262S/T, A261N+I263 S/T, A262N+N264S/T, I263N+K265S/T, K265N+N267S/T, A266N+H268S/T, D276N+P278S/T, P278N+V280S/T, E277N+L279S/T, V280N+N282S/T, Y284S/T, S283N+V285 S/T, Y284N, D292N+K294S/T, K293N+Y295 S/T, E294N, F299S/T, I298N+L300S/T, K301N+G303S/T, F302N, G303N+G305S/T, S304N+Y306S/T, Y306N+S308S/T, R312N+F314S/T, V313N+H315T, F314N+K316S/T, H315N+G317S/T, K316N+R138S/T, G317N, R318N+A320S/T, S319N+L321S/T, A320N+V322T, L321N+L323S/T, V322N+Q324S/T, Y325N+R327S/T, R327N+P329S/T, P329N+V331S/T, L330N+D332S/T, D332N+A334S/T, R333N, A334N+C336S/T, T335N+L337S/T, L337N, R338N, S339N+K341T, T340N+F342T; K341N, F342N+I344S/T, T343N+Y345S/T, Y345N+N347S/T, M348S/T, G352N+H354T, F353N, F353N+E355T, H354N+G356S/T, H354V, H354I, E355T, E355N+G357S/T, G356N+R358T, G357N+D359S/T, R358N, Q362N+D364S/T, V370N; T371V; T3711; E372T, E372N+E374S/T, E374N, G375N, W385N+E387T; G386N+E388T, E388N+A390S/T, A390N+K392T, M391N+G393S/T, K392N+K394S/T, K392V, G393T, G393N+Y395S/T, K394N+G396S/T, R403N+V405S/T, I408S/T, K409N+K411S/T, E410N, K411N+K413S/T, and K413N. In some examples, 1, 2, 3, 4, 5, 6, 7, 8, or more, glycosylation sites are introduced.

Also provided herein are prophylactic subcutaneous methods and regimens that use modified FIX polypeptides containing one or more modifications that eliminates one or more N-glycosylation sites compared to the unmodified FIX polypeptide. For example, N-glycosylation sites at amino acid positions corresponding to N157 or N167 of the mature FIX polypeptide set forth in SEQ ID NO:3 can be eliminated. Exemplary modifications that eliminate an N-glycosylation site include those selected from among modifications corresponding to amino acid replacements N157D, N157Q, N167D, and N167Q. In further examples, the FIX polypeptide contains one or more modifications that eliminates one or more O-glycosylation sites compared to the unmodified FIX polypeptide. For example, O-glycosylation sites that can be eliminated include those at amino acid positions corresponding to positions selected from among S53, S61, T159, and T169, of the mature FIX polypeptide set forth in SEQ ID NO:3. Exemplary modifications that eliminate an N-glycosylation site include those selected from among modifications corresponding to amino acid replacements S53A, S61A, T159A, and T169A. Provided are prophylactic subcutaneous methods and regimens that employ modified FIX polypeptides containing one or more modifications that introduces and/or eliminates one or more sulfation sites, compared to the unmodified FIX polypeptide. In one example, the modified FIX polypeptides contain a modification that eliminates a sulfation site at an amino acid position corresponding to position Y155 of the mature FIX polypeptide set forth in SEQ ID NO:3. Exemplary of such modifications are those that correspond to amino acid replacements Y155H, Y155F, and Y155Q.

Provided are prophylactic subcutaneous methods and regimens that use modified FIX polypeptides containing one or more modifications that introduces and/or eliminates one or more phosphorylation sites, compared to the unmodified FIX polypeptide. In one example, the modified FIX polypeptide contains a modification that eliminates a phosphorylation site at an amino acid position corresponding to position S158 of the mature FIX polypeptide set forth in SEQ ID NO:3. Exemplary of such modifications are those that correspond to the amino acid replacements S158A, S158D, and S158E. Also provided are FIX polypeptides containing one or more modifications that introduces and/or eliminates one or more β-hydroxylation sites compared to the unmodified FIX polypeptide. In one instance, the modified FIX polypeptides contain a modification that eliminates a β-hydroxylation site at an amino acid position corresponding to position D64 of the mature FIX polypeptide set forth in SEQ ID NO:3. Exemplary of such modifications are those that correspond to the amino acid replacements D64N and D64A.

Any of the modified FIX polypeptides provided herein can contain any other mutations known in the art, such as, for example, one or more modifications selected from among amino acid replacements Y1A, Y1C, Y1D, Y1E, Y1G, Y1H, Y1K, Y1N, Y1P, Y1Q, Y1R, Y1S, Y1T, S3T, K5A, K5I, K5L, K5F, K5E, L6A, L6C, L6D, L6E, L6G, L6H, L6K, L6N, L6P, L6Q, L6R, L6S, L6T, L6M, F9A, F9C, F9D, F9E, F9G, F9H, F9K, F9N, F9P, F9Q, F9R, F9S, F9T, F9I, F9M, F9W, V10A, V10C, V10D, V10E, V10G, V10H, V10K, V10N, V10P, V10Q, V10R, V10S, V10T, V10F, V10I, V10K, V10M, V10W, V10Y, Q11E, Q11D, Q11A, Q11C, Q11G, Q11P, G12D, G12E, G12G, G12H, G12K, G12N, G12P, G12Q, G12R, G12S, G12T, N13A, N13C, N13G, N13H, N13P, N13T, L14A, L14C, L14D, L14E, L14G, L14H, L14K, L14N, L14P, L14Q, L14R, L14S, L14T, L14F, L14I, L14M, L14V, L14W, L14Y, E15D, E15H, E15P, R16E, R16A, R16C, R16G, R16P, R16T, E17A, E17C, E17G, E17P, E17T, C18D, C18E, C18G, C18H, C18K, C18N, C18P, C18Q, C18R, C18S, C18T, M19A, M19C, M19D, M19E, M19G, M19H, M19K, M19N, M19P, M19Q, M19R, M19S, M19T, M19F, M19I, M19M, M19V, M19W, M19Y, E20A, E20C, E20G, E20P, E20T, E21A, E21C, E21G, E21P, K22H, K22P, K22T, S24H, S24P, F25A, F25C, F25D, F25E, F25G, F25H, F25K, F25N, F25P, F25Q, F25R, F25S, F25T, F251, F25M, F25W, F25Y, E26A, E26C, E26G, E26P, E27A, E27C, E27G, E27H, E27P, E27S, E27T, A28C, A28D, A28E, A28G, A28H, A28K, A28N, A28P, A28Q, A28R, A28S, A28T, R29A, R29C, R29G, R29P, R29F, E30D, E30H, E30P, V31A, V31C, V31D, V31E, V31G, V31H, V31K, V31N, V31P, V31Q, V31R, V31S, V31T, V31F, V31I, V31W, V31Y, F32A, F32C, F32D, F32E, F32G, F32H, F32K, F32N, F32P, F32Q, F32R, F32S, F32T, E33H, E33N, E33P, E33Q, E33S, E33T, N34E, N34D, N34F, N34I, N34L, T35D, T35E, T35A, T35C, T35G, T35P, F41A, F41C, F41D, F41E, F41G, F41H, F41K, F41N, F41P, F41Q, F41R, F41S, F41T, F41M, F41W, F41Y, W42A, W42C, W42D, W42E, W42G, W42H, W42K, W42N, W42P, W42Q, W42R, W42S, W42T, K43A, K43C, K43G, K43P, Q44P, Q44T, Q44, Y45A, Y45C, Y45D, Y45E, Y45G, Y45H, Y45K, Y45N, Y45P, Y45Q, Y45R, Y45S, Y45T, V46A, V46C, V46D, V46E, V46G, V46H, V46K, V46N, V46P, V46Q, V46R, V46S, V46T, V46F, V46I, V46M, V46W, V46Y, D47A, D47C, D47G, D47H, D47P, D47T, G48D, G48E, G48P, G48T, D49H, D49P, D49Q, D49T, Q50A, Q50C, Q50D, Q50G, Q50H, Q50P, Q50T, C51D, C51E, C51G, C51H, C51K, C51N, C51P, C51Q, C51R, C51S, C51T, E52P, E52T, S53A, S53C, S53G, S53H, S53P, S53T, N54H, N54P, N54T, L57A, L57C, L57D, L57E, L57G, L57H, L57K, L57N, L57P, L57Q, L57R, L57S, L57T, L57F, L57I, L57M, L57W, L57Y, G60C, G60D, G60H, G60P, G60T, C62D, C62H, C62P, K63T, D65H, D65T, I66A, I66C, I66D, I66E, I66G, I66H, I66K, I66N, I66P, I66Q, I66R, I66S, I66T, I66M, I66W, I66Y, Y69A, Y69C, Y69D, Y69E, Y69G, Y69H, Y69K, Y69N, Y69P, Y69Q, Y69R, Y69S, Y69T, C71H, C71P, W72A, W72C, W72D, W72E, W72G, W72H, W72K, W72N, W72P, W72Q, W72R, W72S, W72T, W72I, W72Y, F75A, F75C, F75D, F75E, F75G, F75H, F75K, F75N, F75P, F75Q, F75R, F75S, F75T, F77A, F77C, F77D, F77E, F77G, F77H, F77K, F77N, F77P, F77Q, F77R, F77S, F77T, L84A, L84C, L84D, L84E, L84G, L84H, L84K, L84N, L84P, L84Q, L84R, L84S, L84T, L84M, L84W, L84Y, V86I, V86L, V86M, V86F, V86W, V86Y, V86A, V86C, V86D, V86E, V86G, V86H, V86K, V86N, V86P, V86Q, V86R, V86S, V86T, I90A, I90C, I90D, I90E, I90G, I90H, I90K, I90N, I90P, I90Q, I90R, I90S, I90T, I90M, I90W, K91A, K91C, K91G, K91P, N92A, N92C, N92G, N92P, N92T, G93D, G93E, G93H, G93K, G93N, G93P, G93Q, G93R, G93S, G93T, R94A, R94C, R94G, R94P, C95D, C95E, C95G, C95H, C95K, C95N, C95P, C95Q, C95R, C95S, C95T, E96P, E96T, Q97A, Q97C, Q97G, Q97P, F98A, F98C, F98D, F98E, F98G, F98H, F98K, F98N, F98P, F98Q, F98R, F98S, F98T, F98M, F98W, F98Y, K100A, K100C, K100G, K100P, N101H, N101T, A103D, A103E, A103H, A103K, A103N, A103P, A103Q, A103R, A103S, A103T, D104T, K106H, K106P, K106T, V107A, V107C, V107D, V107E, V107G, V107H, V107K, V107N, V107P, V107Q, V107R, V107S, V107T, V108A, V108C, V108D, V108E, V108G, V108H, V108K, V108N, V108P, V108Q, V108R, V108S, V108T, V108F, V108M, V108W, V108Y, S110A, S110C, S110G, S110P, C111D, C111E, C111H, C111K, C111N, C111P, C111Q, C111R, C111S, C111T, T112A, T112C, T112G, T112P, E113D, E113H, E113P, G114D, G114E, G114H, G114K, G114N, G114P, G114Q, G114R, G114S, G114T, Y115A, Y115C, Y115D, Y115E, Y115G, Y115H, Y115K, Y115N, Y115P, Y115Q, Y115R, Y115S, Y115T, Y115M, Y115W, R116P, R116T, L117A, L117C, L117D, L117E, L117G, L117H, L117K, L117N, L117P, L117Q, L117R, L117S, L117T, A118D, A118E, A118H, A118K, A118N, A118P, A118Q, A118R, A118S, A118T, N120D, N120H, N120P, Q121T, S123H, S123T, V128A, V128C, V128D, V128E, V128G, V128H, V128K, V128N, V128P, V128Q, V128R, V128S, V128T, F130A, F130C, F130D, F130E, F130G, F130H, F130K, F130N, F130P, F130Q, F130R, F130S, F130T, V135A, V135C, V135D, V135E, V135G, V135H, V135K, V135N, V135P, V135Q, V135R, V135S, V135T, V135W, V135Y, V137A, V137C, V137D, V137E, V137G, V137H, V137K, V137N, V137P, V137Q, V137R, V137S, V137T, V137M, V137W, V137Y, S138H, S138T, T140D, T140H, S141T, K142H, K142P, L143A, L143C, L143D, L143E, L143G, L143H, L143K, L143N, L143P, L143Q, L143R, L143S, L143T, L143F, L143I, L143M, L143V, L143W, L143Y, R145H, R145P, R145T, A146P, A146T, T148H, T148P, V149A, V149C, V149D, V149E, V149G, V149H, V149K, V149N, V149P, V149Q, V149R, V149S, V149T, V149F, V149I, V149M, V149W, V149Y, F150A, F150C, F150D, F150E, F150G, F150H, F150K, F150N, F150P, F150Q, F150R, F150S, F150T, F150M, F150W, F150Y, D152A, D152C, D152G, D152P, D152S, D152T, V153A, V153C, V153D, V153E, V153G, V153H, V153K, V153N, V153P, V153Q, V153R, V153S, V153T, V153F, V153I, V153M, V153W, V153Y, D154A, D154C, D154G, D154P, D154Q, D154S, Y155A, Y155C, Y155D, Y155E, Y155G, Y155H, Y155K, Y155N, Y155P, Y155Q, Y155R, Y155S, Y155T, Y155M, Y155V, Y155W, V156A, V156C, V156D, V156E, V156G, V156H, V156K, V156N, V156P, V156Q, V156R, V156S, V156T, V156I, V156M, V156W, V156Y, N157A, N157C, N157G, N157H, N157P, N157Q, N157T, S158H, S158P, S158T, T159A, T159C, T159G, T159P, E160A, E160C, E160G, E160P, A161C, A161D, A161E, A161H, A161K, A161N, A161P, A161Q, A161R, A161S, A161T, E162P, E162T, T163A, T163C, T163G, T163P, I164A, I164C, I164D, I164E, I164G, I164H, I164K, I164N, I164P, I164Q, I164R, I164S, I164T, L165A, L165C, L165D, L165E, L165G, L165H, L165K, L165N, L165P, L165Q, L165R, L165S, L165T, L165M, L165W, L165Y, I168A, I168C, I168D, I168E, I168G, I168H, I168K, I168N, I168P, I168Q, I168R, I168S, I168T, F175A, F175C, F175D, F175E, F175G, F175H, F175K, F175N, F175P, F175Q, F175R, F175S, F175T, F178A, F178C, F178D, F178E, F178G, F178H, F178K, F178N, F178P, F178Q, F178R, F178S, F178T, F178M, F178W, F178Y, T179A, T179C, T179G, T179P, R180A, R180C, R180D, R180G, R180H, R180P, V181A, V181C, V181D, V181E, V181G, V181H, V181K, V181N, V181P, V181Q, V181R, V181S, V181T, V181F, V181I, V181M, V181W, V181Y, V182A, V182C, V182D, V182E, V182G, V182H, V182K, V182N, V182P, V182Q, V182R, V182S, V182T, V182F, V182I, V182M, V182W, V182Y, G183D, G183E, G183H, G183K, G183N, G183P, G183Q, G183S, G183T, G184D, G184E, G184H, G184K, G184N, G184P, G184Q, G184R, G184S, G184T, E185A, E185C, E185G, E185H, E185P, E185T, D186A, D186C, D186G, D186H, D186P, D186T, A187C, A187D, A187E, A187G, A187H, A187K, A187N, A187P, A187Q, A187R, A187S, A187T, K188A, K188C, K188G, K188H, K188P, K188T, G190D, G190E, G190H, G190K, G190N, G190P, G190Q, G190R, G190S, G190T, F192A, F192C, F192D, F192E, F192G, F192H, F192K, F192N, F192P, F192Q, F192R, F192S, F192T, F192W, F192Y, W194A, W194C, W194D, W194E, W194G, W194H, W194K, W194N, W194P, W194Q, W194R, W194S, W194T, Q195H, Q195P, Q195T, V196A, V196C, V196D, V196E, V196G, V196H, V196K, V196N, V196P, V196Q, V196R, V196S, V196T, V196F, V196I, V196M, V196W, V196Y, V197A, V197C, V197D, V197E, V197G, V197H, V197K, V197N, V197P, V197Q, V197R, V197S, V197T, V197F, V197I, V197M, V197W, V197Y, L198A, L198C, L198D, L198E, L198G, L198H, L198K, L198N, L198P, L198Q, L198R, L198S, L198T, L198I, L198Y, N199A, N199C, N199G, N199H, N199P, N199S, N199T, G200P, G200T, K201A, K201C, K201D, K201E, K201G, K201H, K201N, K201P, K201Q, K201S, K201T, V202A, V202C, V202D, V202E, V202G, V202H, V202K, V202N, V202P, V202Q, V202R, V202S, V202T, V202F, V202I, V202M, V202W, V202Y, D203A, D203C, D203G, D203P, D203T, A204C, A204D, A204E, A204G, A204H, A204K, A204N, A204P, A204Q, A204R, A204S, A204T, F205A, F205C, F205D, F205E, F205G, F205H, F205K, F205N, F205P, F205Q, F205R, F205S, F205T, F205M, F205V, F205W, F205Y, G207H, G207P, G208C, G208D, G208E, G208H, G208K, G208N, G208P, G208Q, G208R, G208S, G208T, S209A, S209C, S209G, S209P, I210A, I210C, I210D, I210E, I210G, I210H, I210K, I210N, I210P, I210Q, I210R, I210S, I210T, I210F, I210W, I210Y, V211A, V211C, V211D, V211E, V211G, V211H, V211K, V211N, V211P, V211Q, V211R, V211S, V211T, V211F, V2111, V211M, V211W, N212A, N212C, N212G, N212P, E213H, E213P, E213S, E213T, K214T, W215A, W215C, W215D, W215E, W215G, W215H, W215K, W215N, W215P, W215Q, W215R, W215S, W215T, I216A, I216C, I216D, I216E, I216G, I216H, I216K, I216N, I216P, I216Q, I216R, I216S, I216T, V217A, V217C, V217D, V217E, V217G, V217H, V217K, V217N, V217P, V217Q, V217R, V217S, V217T, V2171, V217Y, A219H, A219P, A219T, V223A, V223C, V223D, V223E, V223G, V223H, V223K, V223N, V223P, V223Q, V223R, V223S, V223T, V223M, V223W, V223Y, G226P, V227A, V227C, V227D, V227E, V227G, V227H, V227K, V227N, V227P, V227Q, V227R, V227S, V227T, V227F, V2271, V227M, V227W, V227Y, K228A, K228C, K228G, K228H, K228P, I229A, I229C, I229D, I229E, I229G, I229H, I229K, I229N, I229P, I229Q, I229R, I229S, I229T, I229M, I229W, I229Y, T230A, T230C, T230G, T230P, V231A, V231C, V231D, V231E, V231G, V231H, V231K, V231N, V231P, V231Q, V231R, V231S, V231T, V232A, V232C, V232D, V232E, V232G, V232H, V232K, V232N, V232P, V232Q, V232R, V232S, V232T, V232F, V2321, V232M, V232W, V232Y, A233C, A233D, A233E, A233G, A233H, A233K, A233N, A233P, A233Q, A233R, A233S, A233T, A233V, G234D, G234E, G234H, G234K, G234N, G234P, G234Q, G234R, G234S, G234T, E235H, E235N, E235P, E235Q, E235S, E235T, H236A, H236C, H236G, H236P, N237A, N237C, N237G, N237P, N237T, I238A, I238C, I238D, I238E, I238G, I238H, I238K, I238N, I238P, I238Q, I238R, I238S, I238T, E239A, E239C, E239G, E239P, E240H, E240T, V250A, V250C, V250D, V250E, V250G, V250H, V250K, V250N, V250P, V250Q, V250R, V250S, V250T, V250M, V250W, V250Y, I251A, I251C, I251D, I251E, I251G, I251H, I251K, I251N, I251P, I251Q, I251R, I251S, I251T, I253A, I253C, I253D, I253E, I253G, I253H, I253K, I253N, I253P, I253Q, I253R, I253S, I253T, I253M, I253W, I253Y, I254A, I254C, I254D, I254E, I254G, I254H, I254K, I254N, I254P, I254Q, I254R, I254S, I254T, P255H, H256P, H256T, H257A, H257C, H257G, H257P, N258P, N258T, Y259A, Y259C, Y259D, Y259E, Y259G, Y259H, Y259K, Y259N, Y259P, Y259Q, Y259R, Y259S, Y259T, Y259M, Y259W, Y259F, N260A, N260C, N260G, N260P, A261D, A261E, A261H, A261K, A261N, A261P, A261Q, A261R, A261S, A261T, A262C, A262D, A262E, A262G, A262H, A262K, A262N, A262P, A262Q, A262R, A262S, A262T, I263A, I263C, I263D, I263E, I263G, I263H, I263K, I263N, I263P, I263Q, I263R, I263S, I263T, I263M, I263V, I263W, I263Y, N264A, N264C, N264D, N264G, N264H, N264P, K265A, K265C, K265G, K265H, K265P, K265T, Y266A, Y266C, Y266D, Y266E, Y266G, Y266H, Y266K, Y266N, Y266P, Y266Q, Y266R, Y266S, Y266T, Y266M, Y266W, N267A, N267C, N267G, N267H, N267P, N267T, H268P, D269A, D269C, D269E, D269G, D269H, D269N, D269P, D269Q, D269S, D269T, I270A, I270C, I270D, I270E, I270G, I270H, I270K, I270N, I270P, I270Q, I270R, I270S, I270T, I270M, I270W, A271C, A271D, A271E, A271G, A271H, A271K, A271N, A271P, A271Q, A271R, A271S, A271T, L272A, L272C, L272D, L272E, L272G, L272H, L272K, L272N, L272P, L272Q, L272R, L272S, L272T, L272F, L273A, L273C, L273D, L273E, L273G, L273H, L273K, L273N, L273P, L273Q, L273R, L273S, L273T, L273F, L273I, L273M, L273V, L273W, L273Y, E274A, E274C, E274G, E274P, E274T, L275A, L275C, L275D, L275E, L275G, L275H, L275K, L275N, L275P, L275Q, L275R, L275S, L275T, L275W, L275Y, D276P, D276S, D276T, E277A, E277C, E277G, E277P, E277V, E277N, E277D, E277E, E277Q, E277H, E277I, E277L, E277M, E277F, E277S, E277T, E277W, E277Y, P278T, L279A, L279C, L279D, L279E, L279G, L279H, L279K, L279N, L279P, L279Q, L279R, L279S, L279T, L279I, L279Y, V280A, V280C, V280D, V280E, V280G, V280H, V280K, V280N, V280P, V280Q, V280R, V280S, V280T, V280F, V280I, V280W, V280Y, L281A, L281C, L281D, L281E, L281G, L281H, L281K, L281N, L281P, L281Q, L281R, L281S, L281T, L281F, L281I, L281V, L281W, L281Y, S283A, S283C, S283G, S283P, Y284A, Y284C, Y284D, Y284E, Y284G, Y284H, Y284K, Y284N, Y284P, Y284Q, Y284R, Y284S, Y284T, Y284M, V285A, V285C, V285D, V285E, V285G, V285H, V285K, V285N, V285P, V285Q, V285R, V285S, V285T, V285M, V285W, V285Y, T286A, T286C, T286G, T286P, I288A, I288C, I288D, I288E, I288G, I288H, I288K, I288N, I288P, I288Q, I288R, I288S, I288T, C289D, C289H, C289P, I290A, I290C, I290D, I290E, I290G, I290H, I290K, I290N, I290P, I290Q, I290R, I290S, I290T, I290Y, A291D, A291E, A291H, A291K, A291N, A291P, A291Q, A291R, A291S, A291T, D292A, D292C, D292G, D292P, D292T, K293H, K293P, K293T, Y295A, Y295C, Y295D, Y295E, Y295G, Y295H, Y295K, Y295N, Y295P, Y295Q, Y295R, Y295S, Y295T, Y295W, T296A, T296C, T296G, T296P, N297A, N297C, N297G, N297P, I298A, I298C, I298D, I298E, I298G, I298H, I298K, I298N, I298P, I298Q, I298R, I298S, I298T, F299A, F299C, F299D, F299E, F299G, F299H, F299K, F299N, F299P, F299Q, F299R, F299S, F299T, L300A, L300C, L300D, L300E, L300G, L300H, L300K, L300N, L300P, L300Q, L300R, L300S, L300T, L300F, L300I, L300M, L300V, L300W, L300Y, K301A, K301C, K301G, K301P, K301T, F302A, F302C, F302D, F302E, F302G, F302H, F302K, F302N, F302P, F302Q, F302R, F302S, F302T, G303H, G303P, G303T, S304A, S304C, S304G, S304P, S304T, G305D, G305E, G305H, G305N, G305P, G305Q, G305S, G305T, Y306A, Y306C, Y306D, Y306E, Y306G, Y306H, Y306K, Y306N, Y306P, Y306Q, Y306R, Y306S, Y306T, V307A, V307C, V307D, V307E, V307G, V307H, V307K, V307N, V307P, V307Q, V307R, V307S, V307T, S308P, S308T, W310A, W310C, W310D, W310E, W310G, W310H, W310K, W310N, W310P, W310Q, W310R, W310S, W310T, G311H, V313A, V313C, V313D, V313E, V313G, V313H, V313K, V313N, V313P, V313Q, V313R, V313S, V313T, F314A, F314C, F314D, F314E, F314G, F314H, F314K, F314N, F314P, F314Q, F314R, F314S, F314T, F314M, F314W, F314Y, H315A, H315C, H315G, H315P, K316A, K316C, K316G, K316P, G317C, G317D, G317E, G317H, G317K, G317N, G317P, G317Q, G317R, G317S, G317T, R318A, R318C, R318G, R318P, S319D, S319H, S319N, S319P, S319Q, A320C, A320D, A320E, A320G, A320H, A320K, A320N, A320P, A320Q, A320R, A320S, A320T, L321A, L321C, L321D, L321E, L321G, L321H, L321K, L321N, L321P, L321Q, L321R, L321S, L321T, V322A, V322C, V322D, V322E, V322G, V322H, V322K, V322N, V322P, V322Q, V322R, V322S, V322T, V322W, V322Y, L323A, L323C, L323D, L323E, L323G, L323H, L323K, L323N, L323P, L323Q, L323R, L323S, L323T, L323F, L323I, L323M, L323V, L323W, L323Y, Q324A, Q324C, Q324G, Q324P, Y325A, Y325C, Y325D, Y325E, Y325G, Y325H, Y325K, Y325N, Y325P, Y325Q, Y325R, Y325S, Y325T, Y325W, L326A, L326C, L326D, L326E, L326G, L326H, L326K, L326N, L326P, L326Q, L326R, L326S, L326T, L326F, L326I, L326M, L326V, L326W, L326Y, R327A, R327C, R327G, R327H, R327P, V328A, V328C, V328D, V328E, V328G, V328H, V328K, V328N, V328P, V328Q, V328R, V328S, V328T, V328F, V328I, V328M, V328W, V328Y, L330A, L330C, L330D, L330E, L330G, L330H, L330K, L330N, L330P, L330Q, L330R, L330S, L330T, L330F, L330I, L330V, L330W, L330Y, V331A, V331C, V331D, V331E, V331G, V331H, V331K, V331N, V331P, V331Q, V331R, V331S, V331T, V331F, V331I, V331M, V331W, V331Y, D332A, D332C, D332G, D332P, R333A, R333C, R333D, R333E, R333G, R333H, R333N, R333P, R333Q, R333R, R333S, R333T, A334C, A334D, A334E, A334G, A334H, A334K, A334N, A334P, A334Q, A334R, A334S, A334T, T335A, T335C, T335G, T335P, C336D, C336E, C336H, C336K, C336N, C336P, C336Q, C336R, C336S, C336T, L337A, L337C, L337D, L337E, L337G, L337H, L337K, L337N, L337P, L337Q, L337R, L337S, L337T, R338A, R338E, R338V, R338T, R338C, R338G, R338P, R338I, R338F, R338W, R338S, S339P, S339T, K341A, K341C, K341G, K341P, F342A, F342C, F342D, F342E, F342G, F342H, F342K, F342N, F342P, F342Q, F342R, F342S, F342T, F342M, F342W, T343A, T343C, T343G, T343P, I344A, I344C, I344D, I344E, I344G, I344H, I344K, I344N, I344P, I344Q, I344R, I344S, I344T, Y345F, Y345A, Y345C, Y345D, Y345E, Y345G, Y345H, Y345K, Y345N, Y345P, Y345Q, Y345R, Y345S, Y345T, Y345M, Y345W, N346A, N346C, N346G, N346P, N347H, N347P, M348A, M348C, M348D, M348E, M348G, M348H, M348K, M348N, M348P, M348Q, M348R, M348S, M348T, F349A, F349C, F349D, F349E, F349G, F349H, F349K, F349N, F349P, F349Q, F349R, F349S, F349T, F349I, F349M, F349W, F349Y, C350D, C350H, C350P, C350T, A351E, A351H, A351N, A351P, A351Q, A351R, A351S, A351T, G352A, G352C, G352P, F353A, F353C, F353D, F353E, F353G, F353H, F353K, F353N, F353P, F353Q, F353R, F353S, F353T, F353I, F353M, F353W, H354A, H354C, H354G, H354P, E355A, E355C, E355D, E355G, E355H, E355K, E355N, E355P, E355Q, E355S, E355T, G356D, G356E, G356H, G356K, G356N, G356P, G356Q, G356R, G356S, G356T, G357D, G357E, G357H, G357K, G357N, G357P, G357Q, G357R, G357S, G357T, R358D, R358E, R358H, R358K, R358N, R358P, R358Q, R358R, R358S, R358T, D359A, D359C, D359G, D359P, D359Q, D359S, D359T, S360A, S360C, S360G, S360P, C361D, C361E, C361H, C361K, C361N, C361P, C361Q, C361R, C361S, C361T, V370A, V370C, V370D, V370E, V370G, V370H, V370K, V370N, V370P, V370Q, V370R, V370S, V370T, V370W, V370Y, V373A, V373C, V373D, V373E, V373G, V373H, V373K, V373N, V373P, V373Q, V373R, V373S, V373T, V373F, V373I, V373M, V373W, E374A, E374C, E374G, E374P, G375H, S377A, S377C, S377G, S377P, F378A, F378C, F378D, F378E, F378G, F378H, F378K, F378N, F378P, F378Q, F378R, F378S, F378T, F378W, L379A, L379C, L379D, L379E, L379G, L379H, L379K, L379N, L379P, L379Q, L379R, L379S, L379T, L379I, L379M, L379W, L379Y, T380A, T380C, T380G, T380P, G381D, G381E, G381H, G381K, G381N, G381P, G381Q, G381R, G381S, G381T, I382A, I382C, I382D, I382E, I382G, I382H, I382K, I382N, I382P, I382Q, I382R, I382S, I382T, I382M, I382W, I382Y, I383A, I383C, I383D, I383E, I383G, I383H, I383K, I383N, I383P, I383Q, I383R, I383S, I383T, I383V, S384A, S384C, S384G, S384P, W385A, W385C, W385D, W385E, W385G, W385H, W385K, W385N, W385P, W385Q, W385R, W385S, W385T, W385M, E387A, E387C, E387G, E387H, E387P, E387T, E388H, E388N, E388G, E388P, E388Q, E388T, A390C, A390D, A390E, A390G, A390H, A390K, A390N, A390P, A390Q, A390R, A390S, M391A, M391C, M391D, M391E, M391G, M391H, M391K, M391N, M391P, M391Q, M391R, M391S, M391T, M391F, M391I, M391W, M391Y, K392A, K392C, K392G, K392P, G393C, G393D, G393E, G393H, G393K, G393N, G393P, G393Q, G393R, G393S, G393T, Y395A, Y395C, Y395D, Y395E, Y395G, Y395H, Y395K, Y395N, Y395P, Y395Q, Y395R, Y395S, Y395T, Y398A, Y398C, Y398D, Y398E, Y398G, Y398H, Y398K, Y398N, Y398P, Y398Q, Y398R, Y398S, Y398T, K400H, V401A, V401C, V401D, V401E, V401G, V401H, V401K, V401N, V401P, V401Q, V401R, V401S, V401T, V401F, V401I, V401M, V401W, V401Y, S402A, S402C, S402G, S402P, R403A, R403C, R403G, R403P, R403T, Y404A, Y404C, Y404D, Y404E, Y404G, Y404H, Y404K, Y404N, Y404P, Y404Q, Y404R, Y404S, Y404T, V405A, V405C, V405D, V405E, V405G, V405H, V405K, V405N, V405P, V405Q, V405R, V405S, V405T, V405W, V405Y, N406F, N406H, N406I, N406L, N406P, N406W, N406Y, W407D, W407E, W407F, W407H, W407I, W407K, W407N, W407P, W407Q, W407R, W407S, W407T, W407Y, I408D, I408E, I408H, I408K, I408N, I408P, I408Q, I408R, I408S, I408T, K409F, K409H, K409I, K409P, K409T, K409V, K409W, K409Y, E410H, K411A, K411C, K411G, K411I, K411P, K411T, K411V, K411W, K411Y, K413T, Y1I, S3Q, S3H, S3N, G4Q, G4H, G4N, K5N, K5Q, L6I, L6V, E7Q, E7H, E7N, E8Q, E8H, E8N, F9V, E15Q, E15N, R16H, R16Q, E17Q, E17H, E17N, E20Q, E20H, E20N, E21Q, E21H, E21N, K22N, K22Q, S24Q, S24N, F25V, E26Q, E26H, E26N, E27Q, E27N, R29H, R29Q, E30Q, E30N, F32I, F32V, T35Q, T35H, T35N, E36Q, E36H, E36N, R37H, R37Q, T38Q, T38H, T38N, T39Q, T39H, T39N, E40Q, E40H, E40N, F41I, F41V, K43N, K43Q, Y45I, D47N, D47Q, G48Q, G48H, G48N, D49N, E52Q, E52H, E52N, S53Q, S53N, P55A, P55S, L57V, N58Q, N58S, G59Q, G59H, G59N, G60Q, G60N, S61Q, S61H, S61N, K63N, K63Q, D64N, D64Q, D65N, D65Q, S68Q, S68H, S68N, Y69I, E70Q, E70H, E70N, P74A, P74S, F75I, F75V, G76Q, G76H, G76N, F77I, F77V, E78Q, E78H, E78N, G79Q, G79H, G79N, K80N, K80Q, E83Q, E83H, E83N, L84I, L84V, D85N, D85Q, T87Q, T87H, T87N, K91N, K91Q, N92Q, N92S, R94H, R94Q, E96Q, E96H, E96N, F98I, F98V, K100N, K100Q, S102Q, S102H, S102N, D104N, D104Q, K106N, K106Q, S110Q, S110H, S110N, T112Q, T112H, T112N, E113Q, E113N, Y115I, R116H, R116Q, L117I, L117V, E119Q, E119H, E119N, K122N, K122Q, S123Q, S123N, E125Q, E125H, E125N, P126A, P126S, A127Q, A127H, A127N, P129A, P129S, P131A, P131S, G133Q, G133H, G133N, R134H, R134Q, S136Q, S136H, S136N, S138Q, S138N, T140Q, T140N, S141Q, S141H, S141N, K142N, K142Q, T144Q, T144H, T144N, R145Q, A146Q, A146H, A146N, E147Q, E147H, E147N, T148Q, T148N, P151A, P151S, D152N, D152Q, D154N, Y155I, S158Q, S158N, T159Q, T159H, T159N, E160Q, E160H, E160N, E162Q, E162H, E162N, T163Q, T163H, T163N, L165I, L165V, D166N, D166Q, T169Q, T169H, T169N, S171Q, S171H, S171N, T172Q, T172H, T172N, S174Q, S174H, S174N, F175I, F175V, D177N, D177Q, F178I, F178V, T179Q, T179H, T179N, R180Q, E185Q, E185N, D186N, D186Q, K188N, K188Q, P189A, P189S, F192I, F192V, F192IH, P193A, P193S, W194I, L198V, N199Q, G200Q, G200H, G200N, D203N, D203Q, F205I, G207Q, G207N, S209Q, S209H, S209N, E213Q, E213N, K214N, K214Q, T218Q, T218H, T218N, A219Q, A219N, A220Q, A220H, A220N, E224Q, E224H, E224N, T225Q, T225H, T225N, G226Q, G226H, G226N, K228N, K228Q, T230Q, T230H, T230N, E239Q, E239H, E239N, E240Q, E240N, T241Q, T241H, T241N, E242Q, E242H, E242N, T244Q, T244H, T244N, E245Q, E245H, E245N, K247N, K247Q, R248H, R248Q, R252H, R252Q, P255A, P255S, Y259I, K265N, K265Q, Y266I, L272I, L272V, E274Q, E274H, E274N, L275I, L275V, D276N, D276Q, E277Q, E277H, E277N, P278A, P278S, L279V, S283Q, S283H, S283N, Y284I, T286Q, T286H, T286N, P287A, P287S, D292N, D292Q, K293N, K293Q, E294Q, E294H, E294N, Y295I, T296Q, T296H, T296N, F299I, F299V, K301N, K301Q, F302I, F302V, G303Q, G303N, S304Q, S304H, S304N, Y306I, S308Q, S308H, S308N, G309Q, G309H, G309N, G311Q, G311N, R312H, R312Q, F314I, F314V, K316N, K316Q, R318H, R318Q, L321I, L321V, Y325I, R327Q, P329A, P329S, D332N, D332Q, T335Q, T335H, T335N, L337I, L337V, R338H, R338Q, S339Q, S339H, S339N, T340Q, T340H, T340N, K341N, K341Q, F342I, F342V, T343Q, T343H, T343N, Y345I, M348I, M348V, F349V, G352Q, G352H, G352N, F353V, D359N, S360Q, S360H, S360N, G363Q, G363H, G363N, D364N, D364Q, S365Q, S365H, S365N, G366Q, G366H, G366N, G367Q, G367H, G367N, P368A, P368S, T371Q, T371H, T371N, E372Q, E372H, E372N, E374Q, E374H, E374N, G375Q, G375N, T376Q, T376H, T376N, S377Q, S377H, S377N, F378I, F378V, L379V, T380Q, T380H, T380N, S384Q, S384H, S384N, G386Q, G386H, G386N, E387Q, E387N, M391V, K392N, K392Q, K394N, K394Q, Y395I, G396Q, G396H, G396N, I397Q, I397H, I397N, Y398I, T399Q, T399H, T399N, K400N, K400Q, S402Q, S402H, S402N, R403H, R403Q, Y404I, K409N, K409Q, E410Q, E410N, K411N, K411Q, T412Q, T412H, T412N, K413N, K413Q, L414I, L414V, T415Q, T415H, T415N, R252A, H268A, K293A, K400A, R403A, R403E, and K411A.

In some instances, the modified FIX polypeptides for use in the prophylactic subcutaneous methods and regimens exhibit increased resistance to antithrombin III (ATIII), heparin and/or the AT-III/heparin complex, compared with the unmodified FIX polypeptide. For example, the modified FIX polypeptides can exhibit at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, increased resistance to antithrombin III and/or heparin, compared with the unmodified FIX polypeptide. In further instances, the modified FIX polypeptides exhibit increased catalytic activity compared with the unmodified FIX polypeptide. This can be in the presence or absence of FVIIIa. For example, the modified FIX polypeptides can exhibit at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, catalytic activity compared to an unmodified FIX polypeptide.

The modified FIX polypeptides further can exhibit improved pharmacokinetic properties compared with the unmodified FIX polypeptide, such as, for example, decreased clearance (e.g., at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99%, of the clearance of an unmodified FIX polypeptide), altered volume of distribution (e.g., decreased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99%, of the volume of distribution of an unmodified FIX polypeptide, or increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the volume of distribution of an unmodified FIX polypeptide), increased in vivo recovery (e.g., by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the in vivo recovery of an unmodified FIX polypeptide), increased total modified FIX polypeptide exposure in vivo (e.g., increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the total exposure in vivo of an unmodified FIX polypeptide), increased serum half-life (e.g., by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the serum half-life of an unmodified FIX polypeptide), and/or increased mean resonance time (MRT) compared to the unmodified FIX polypeptide (e.g., increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, of the MRT in vivo of an unmodified FIX polypeptide). In some instances, wherein the improved pharmacokinetic property is increased serum half-life, the serum half-life is α, β or γ phase.

In some instances, the modified FIX polypeptides exhibit increased procoagulant activity compared with the unmodified FIX polypeptide, such as, for example, at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more, than the procoagulant activity of an unmodified FIX polypeptide.

In some examples, the unmodified FIX polypeptide has a sequence of amino acids set forth in SEQ ID NO:3. Thus, provided herein are prophylactic subcutaneous methods and regimens using modified FIX polypeptides having a sequence of amino acids set forth in any of SEQ ID NOs: 75-272. In other examples, the unmodified FIX polypeptide is a variant of the polypeptide set forth in SEQ ID NO:3, such as an allelic or species variant having at least 40%, 50%, 60%, 70%, 80%, 90%, 91%, 92%, 93%, 94%, 95%, 96%, 97%, 98%, or 99%, sequence identity to the polypeptide set forth in SEQ ID NO: 3, excluding the modification(s).

Also intended for use in the prophylactic subcutaneous methods and regimens herein are all forms of the modified FIX polypeptides, including single-chain and two-chain FIX polypeptides, and active or activated FIX polypeptides. In some examples, activation is effected by proteolytic cleavage by Factor IX (FIXa), or by the Tissue Factor/Factor VIIa complex.

In some examples, the modified FIX polypeptides have only the primary sequence modified by insertion, deletion, or replacement of amino acid residues. In other examples, there is a chemical modification or a post-translational modification (e.g., the modified FIX polypeptides are glycosylated, carboxylated, hydroxylated, sulfated, phosphorylated, albuminated, or conjugated to a polyethylene glycol (PEG) moiety). The modified FIX polypeptides can be modified to have extended half-life. For example, the modified FIX polypeptides can be hyperglycosylated and/or PEGylated, and/or albuminated. The FIX polypeptides can be chimeric or fusion FIX polypeptides, such as by inclusion of a multimerization domain, such as an Fc domain.

The modified FIX polypeptides can contain 1, 2, 3, 4, 5, 6, 7, 8, 9, 10, or more, modifications, or 1, 2, 3, 4, 5, 6, 7, 8, 9, 10, 20, 30, 40, 50, or 60, or more, modifications, so long as the polypeptide retains at least one FIX activity (e.g., Factor VIIIa binding, Factor X binding, phospholipid binding, and/or coagulant activity) of the unmodified FIX polypeptide. For example, the modified FIX polypeptide can retain at least about or 1%, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more, of an activity of the unmodified FIX polypeptide. In some examples, the activities that are retained are increased compared to the unmodified FIX polypeptide. In other examples, the activities that are retained are decreased compared to the unmodified FIX polypeptide. The activities can be measured in vitro, ex vivo, or in vivo.

Kits containing any of the pharmaceutical compositions provided herein, a device for administration of the composition and, optionally, instructions for subcutaneous administration, also are provided. The compositions can be provided in syringes or other such devices for single dosage administration.

### BRIEF DESCRIPTION OF THE DRAWINGS

FIG. 1 depicts the coagulation cascade. The figure shows the intrinsic pathway and the extrinsic pathway of coagulation for the independent production of FXa, and convergence of the pathways to a common pathway to generate thrombin and fibrin for the formation of a clot. These pathways are interconnected. The figure depicts the order of molecules involved in the activation cascade in which a zymogen is converted to an activated protease by cleavage of one or more peptide bonds. The activated protease then serves as the activating protease for the next zymogen molecule in the cascade, ultimately resulting in clot formation.

FIG. 2 depicts the cell based model of coagulation (see, e.g., Hoffman et al. (2001) J. Thromb. Haemost. 85:958-965). The figure depicts the coagulation events as being separated into three phases, where initiation of coagulation is effected by the activation of FX to FXa by the TF/FVIIa complex on the TF-bearing cell, resulting in the generation of a small amount of thrombin after activation by FXa/FVa. Amplification takes place when thrombin binds to and activates the platelets, and initiates the activation of sufficient quantities of the appropriate coagulation factors to form the FVIIIa/FIXa and FVa/FXa complexes. Propagation of coagulation occurs on the surface of large numbers of activated platelets at the site of injury, resulting in a burst of thrombin generation that is sufficiently large to generate enough fibrin from fibrinogen to establish a clot at the site of injury.

FIGS. 3A-3D provide an alignment of various Factor IX polypeptides, including species variants and modified Factor IX polypeptides (SEQ ID NOs: 2-5, 14, 20, 172, 267, 247, 325, 346, 347, 360, 365, 366, 406). Also included are SEQ ID NO:6 from U.S. Pat. No. 7,700,734, containing mutations V86A/E277A/R338A, and SEQ ID NO:2 from U.S. Pat. No. 7,125,841. An “*” means that the residues or nucleotides in that column are identical in all sequences in the alignment; a “:” means that conserved substitutions have been observed; and a “.” means that semi-conserved substitutions are observed. As described herein, residues corresponding to positions in SEQ ID NO:3 can be determined by alignment with SEQ ID NO:3. Residues corresponding to Y155, R318, R338, T343, R403, and E410 are indicated in boxed text.

FIG. 4 depicts the primary amino acid sequence of the mature form of a variant FIX polypeptide that has three mutations: R318Y/R338E/T343R (see, SEQ ID NO:394), which is a mature form of FIX that has 415 amino acids, and includes 3 point mutations introduced into 2 distinct, solvent exposed surface loops of the FIX protein. The FIX polypeptide of SEQ ID NO:394 also is referred to herein as CB2679d and/or ISU304.

FIG. 5 illustrates that structure and domains of the mature form of FIX (of SEQ ID NO:394) that has three mutations: R318Y/R338E/T343R (CB2679d).

FIG. 6 shows that the modified FIX polypeptide of SEQ ID NO:394, with the replacements R318Y/R338E/T343R, exhibits enhanced activity in vitro and in vivo. In vitro, the FIX of SEQ ID NO:394, with the replacements R318Y/R338E/T343R, exhibits approximately 3-fold enhanced catalytic efficiency for the activation of FX, 10-fold enhanced affinity for FVIIIa, and 15-fold resistance to inhibition by ATIII. In vivo, the FIX of SEQ ID NO:394, with the replacements R318Y/R338E/T343R, displays 20-fold enhanced potency for inhibition of bleeding in a standard murine hemophilia tail cut model, a 17-fold reduction in activated partial thromboplastin time (aPTT), and an 8-fold prolonged correction of aPTT activity compared with BeneFIX® FIX.

FIG. 7 depicts a schematic of the nucleic acid construct, including the ITRs, for encapsulation into the capsids described herein.

FIGS. 8A-8C depict an alignment between the human FIX containing the replacements R318Y/T343R/R338E and optimized for expression in human at a threshold of 1.0 (set forth in SEQ ID NO: 532, top), and human FIX containing the replacements R318Y/T343R/R338E and optimized for expression in mouse (set forth in SEQ ID NO: 560, bottom). The consensus sequence is listed in the top row. Highlighted nucleotides in the mouse optimized and human optimized sequences designate differences between the two. The alignments demonstrate 91.4% identity between the two sequences.

FIGS. 9A-9D depict alignment between and among the human wild-type, modified FIX (with the replacements R318Y/T343R/R338E), and FIX Padua (with the replacement R338L), and forms that were optimized for expression in human at a threshold of 0.5, 0.8, and 1.0, and optimized for expression in mouse. The consensus sequence is listed above the other rows. The top row is human wild-type FIX optimized for expression in mouse (SEQ ID NO: 558). The second row is human wild type FIX (SEQ ID NO: 472). The third, fourth, and fifth rows are human wild-type FIX optimized for expression in human at a threshold of 0.5 (SEQ ID NO: 518), 0.8 (SEQ ID NO: 519), and 1.0 (SEQ ID NO: 520), respectively. The sixth row is human FIX with the replacement R338L (SEQ ID NO: 474). The seventh, eighth, and ninth rows are human FIX with the replacement R338L and optimized for expression in human at a threshold of 0.5 (SEQ ID NO: 524), 0.8 (SEQ ID NO: 525), and 1.0 (SEQ ID NO: 526), respectively. The tenth row is human modified FIX with the replacements R318Y/T343R/R338E (SEQ ID NO: 473). The eleventh, twelfth, and thirteenth rows are human modified FIX with the replacements R318Y/T343R/R338E and optimized for expression in human at a threshold of 0.5 (SEQ ID NO: 530), 0.8 (SEQ ID NO: 531), and 1.0 (SEQ ID NO: 532), respectively. Highlighted nucleotides in the mouse optimized and human optimized sequences designate differences between the two.

FIG. 10 depicts the vector map of the vector used to introduce the construct for encapsulation into the AAV capsid. The construct includes two ITRs, ApoE/C1 gene locus (enhancer), huSerpin A antitrypsin liver promoter (partial), Kozak sequence, Factor IX Signal Sequences, partial Factor IX intron I, Factor IX propeptide and mature polypeptide, and a bGH polyadenylation signal. The vector backbone contains an ampicillin resistance gene, CAP binding site, lac operon promoter, and ColE1/pMB1/pBR322/pUC origin of replication.

### DETAILED DESCRIPTION

Outline

| | |
|---|---|
| A. | Definitions |
| B. | Hemostasis and Role of Factor IX |

| | |
|---|---|
| 1. | Platelet Adhesion and Aggregation |
| 2. | Coagulation Cascade |

| | |
|---|---|
| a. | Initiation |
| b. | Amplification |
| c. | Propagation |

| | |
|---|---|
| 3. | Regulation of Coagulation |

| | |
|---|---|
| C. | Factor IX (FIX) Structure and Function |

| | |
|---|---|
| 1. | FIX Structure |
| 2. | FIX Post-Translational Modification |
| 3. | FIX Activation |
| 4. | FIX Function |
| 5. | FIX as a Biopharmaceutical |

| | |
|---|---|
| D. | AAV Gene Therapy Vectors and Constructs for Gene Therapy |
| E. | Modified FIX Polypeptides |

| | |
|---|---|
| 1. | Exemplary Amino Acid Replacements |

| | |
|---|---|
| a. | Altered Glycosylation |

| | |
|---|---|
| i. | Advantages of Glycosylation |
| ii. | Exemplary Modified FIX Polypeptides with Altered Glycosylation |

| | |
|---|---|
| b. | Increased Resistance to AT-III and Heparin |

| | |
|---|---|
| i. | AT-III |
| ii. | Heparin |
| iii. | Exemplary FIX Polypeptides with Increased Resistance to AT-III and Heparin |

| | |
|---|---|
| c. | Mutations to Increase Catalytic Activity |
| d. | Mutations to Decrease LRP Binding |
| e. | Other Mutations to Alter Post-Translational Modification |

| | |
|---|---|
| 2. | Combination Modifications |

| | |
|---|---|
| a. | Modifications to Increase Activity |
| b. | Modifications that Increase Affinity for Phospholipids or Reduce Binding to Collagen |
| c. | Additional Modifications to Increase Resistance to Inhibitors |
| d. | Additional Modifications to Alter Glycosylation |
| e. | Modifications to Increase Resistance to Proteases |
| f. | Modifications to Reduce Immunogenicity |
| g. | Exemplary Combination Modifications |

| | |
|---|---|
| 3. | Conjugates and Fusion Proteins |

| | |
|---|---|
| F. | Production of FIX Polypeptides |
| G. | Assessing Modified FIX Polypeptide Activities |

| | |
|---|---|
| 1. | In Vitro Assays |

| | |
|---|---|
| a. | Glycosylation |
| b. | Other Post-Translational Modifications |
| c. | Proteolytic Activity |
| d. | Coagulation Activity |
| e. | Binding to and/or Inhibition by Other Proteins and Molecules |
| f. | Phospholipid Affinity |

| | |
|---|---|
| 2. | Non-Human Animal Models |
| 3. | Clinical Assays |

| | |
|---|---|
| H. | Formulation and Administration |

| | |
|---|---|
| 1. | Formulations |
| 2. | Dosages |
| 3. | Administration of the Vectors Encoding Modified FIX Polypeptides |

| | |
|---|---|
| I. | Therapeutic Uses |

| | |
|---|---|
| 1. | Hemophilia |
| 2. | Pathophysiology |
| 3. | Clinical Characteristics |
| 4. | Hemophilia B |

| | |
|---|---|
| J. | Combination Therapies |
| K. | Examples |

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, coagulation pathway or coagulation cascade refers to the series of activation events that leads to the formation of an insoluble fibrin clot. In the coagulation cascade or pathway, an inactive protein of a serine protease (also called a zymogen) is converted to an active protease by cleavage of one or more peptide bonds, which then serves as the activating protease for the next zymogen molecule in the cascade. In the final proteolytic step of the cascade, fibrinogen is proteolytically cleaved by thrombin to fibrin, which is then cross-linked at the site of injury to form a clot.

As used herein, “hemostasis” refers to the stopping of bleeding or blood flow in an organ or body part. The term hemostasis can encompass the entire process of blood clotting to prevent blood loss following blood vessel injury to subsequent dissolution of the blood clot following tissue repair.

As used herein, “clotting” or “coagulation” refers to the formation of an insoluble fibrin clot, or the process by which the coagulation factors of the blood interact in the coagulation cascade, ultimately resulting in the formation of an insoluble fibrin clot.

As used herein, a “protease” is an enzyme that catalyzes the hydrolysis of covalent peptidic bonds. These designations include zymogen forms and activated single-, two- and multiple-chain forms thereof. For clarity, reference to proteases refer to all forms. Proteases include, for example, serine proteases, cysteine proteases, aspartic proteases, threonine and metallo-proteases depending on the catalytic activity of their active site and mechanism of cleaving peptide bonds of a target substrate.

As used herein, serine proteases or serine endopeptidases refers to a class of peptidases, which are characterized by the presence of a serine residue in the active site of the enzyme. Serine proteases participate in a wide range of functions in the body, including blood clotting and inflammation, as well as functioning as digestive enzymes in prokaryotes and eukaryotes. The mechanism of cleavage by serine proteases is based on nucleophilic attack of a targeted peptidic bond by a serine. Cysteine, threonine or water molecules associated with aspartate or metals also can play this role. Aligned side chains of serine, histidine and aspartate form a catalytic triad common to most serine proteases. The active site of serine proteases is shaped as a cleft where the polypeptide substrate binds.

As used herein, a “factor IX” or FIX polypeptide refers to any factor IX polypeptide including, but not limited to, a recombinantly produced polypeptide, a synthetically produced polypeptide and a factor IX polypeptide extracted or isolated from cells or tissues including, but not limited to, liver and blood. Alternative names that are used interchangeably for factor IX include Factor 9, Christmas factor, plasma thromboplastin component (PTC), coagulation factor IX, and serum factor IX. Abbreviations for factor IX include FIX and F9. Factor IX includes related polypeptides from different species including, but not limited to animals of human and non-human origin. Human factor IX (hFIX) includes factor IX, allelic variant isoforms (such as the allelic variant having a T148A (SEQ ID NO: 20 or 325) or T412P mutation), synthetic molecules from nucleic acids, protein isolated from human tissue and cells, and modified forms thereof. Exemplary unmodified mature human factor IX polypeptides include, but are not limited to, unmodified and wild-type native factor IX polypeptides (such as the polypeptide containing a sequence set forth in SEQ ID NO:3) and the unmodified and wild-type precursor factor IX polypeptide that includes a propeptide (Pro) and/or a signal peptide (such as, the precursor FIX polypeptide that has the sequence set forth in SEQ ID NO:2). One of skill in the art would recognize that the referenced positions of the mature factor IX polypeptide (SEQ ID NO:3) differ by 46 amino acid residues when compared to the precursor FIX polypeptide SEQ ID NO:2, which is the factor IX polypeptide containing the signal peptide and propeptide sequences. Thus, the first amino acid residue of SEQ ID NO:3 “corresponds to” the forty-seventh (47^{th}) amino acid residue of SEQ ID NO:2.

The term “factor IX” also encompasses the activated form of the factor IX polypeptide, called factor IXa (FIXa), containing the FIX light chain (corresponding to amino acids 47-191 of SEQ ID NO:2, and amino acids 1-145 of SEQ ID NO:3) and FIX heavy chain (corresponding to amino acids 227-461 of SEQ ID NO:2, and amino acids 181-415 of SEQ ID NO:3) linked by a disulfide bond between residues 132C and 289C (corresponding to the mature FIX polypeptide set forth in SEQ ID NO:3). FIXa is produced from a mature FIX polypeptide (e.g., that set forth in SEQ ID NO:3) by proteolytic cleavage after amino acid residues R145 and R180. Proteolytic cleavage can be carried out, for example, by activated factor XI (FXIa) or the tissue factor/activated factor VII (TF/FVIIa) complex. The FIX polypeptides provided herein can be further modified, such as by chemical modification or post-translational modification. Such modifications include, but are not limited to, glycosylation, PEGylation, albumination, farnesylation, carboxylation, hydroxylation, phosphorylation, and other polypeptide modifications known in the art.

Factor IX includes factor IX from any species, including human and non-human species. FIX polypeptides of non-human origin include, but are not limited to, murine, canine, feline, leporine, avian, bovine, ovine, porcine, equine, piscine, ranine, and other primate factor IX polypeptides. Exemplary FIX polypeptides of non-human origin include, for example, chimpanzee (Pan troglodytes, SEQ ID NO:4), rhesus macaque (Macaca mulatta, SEQ ID NO:5), mouse (Mus musculus, SEQ ID NO:6), rat (Rattus norvegicus, SEQ ID NO:7), Guinea pig (Cavia porcellus, SEQ ID NO:8), pig (Sus scrofa, SEQ ID NO:9), dog (Canis familiaris, SEQ ID NO:10), cat (Felis catus, SEQ ID NO:11), rabbit (Oryctolagus cuniculus, SEQ ID NO:12), chicken (Gallus gallus, SEQ ID NO:13), cow (Bos Taurus, SEQ ID NO:14), sheep (Ovis aries, SEQ ID NO:15), frog (Xenopus tropicalis, SEQ ID NO:16), zebrafish (Danio rerio, SEQ ID NO:17), and Japanese pufferfish (Takifugu rubripes, SEQ ID NO:18).

Reference to FIX polypeptides also includes precursor polypeptides and mature FIX polypeptides in single-chain or two-chain forms, truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptide set forth in SEQ ID NO:2 or the mature form thereof (SEQ ID NO:3). Included are modified FIX polypeptides, such as those of SEQ ID NOs: 75-272 and 326-417, and variants thereof. Also included are those that retain at least an activity of a FIX, such as FVIIIa binding, Factor X binding, phospholipid binding, and/or coagulant activity of a FIX polypeptide. By retaining activity, the activity can be altered, such as reduced or increased, as compared to a wild-type FIX so long as the level of activity retained is sufficient to yield a detectable effect. FIX polypeptides include, but are not limited to, tissue-specific isoforms and allelic variants thereof, synthetic molecules prepared by translation of nucleic acids, proteins generated by chemical synthesis, such as syntheses that include ligation of shorter polypeptides, through recombinant methods, proteins isolated from human and non-human tissue and cells, chimeric FIX polypeptides and modified forms thereof. FIX polypeptides also include fragments or portions of FIX that are of sufficient length or include appropriate regions to retain at least one activity (upon activation if needed) of a full-length mature polypeptide. FIX polypeptides also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, multimerization conjugation (i.e., Fc domain) and other polypeptide modifications known in the art.

As used herein, “corresponding residues” refers to residues that occur at aligned loci. Related or variant polypeptides are aligned by any method known to those of skill in the art. Such methods typically maximize matches, and include methods such as using manual alignments and by using the numerous alignment programs available (for example, BLASTP) and others known to those of skill in the art. By aligning the sequences of polypeptides, one skilled in the art can identify corresponding residues, using conserved and identical amino acid residues as guides. For example, by aligning the sequences of Factor IX polypeptides, one of skill in the art can identify corresponding residues, using conserved and identical amino acid residues as guides. For example, the tyrosine in amino acid position 1 (Y1) of SEQ ID NO:3 (mature factor IX) corresponds to the tyrosine in amino acid position 47 (Y47) of SEQ ID NO:2. In other instances, corresponding regions can be identified. For example, the Gla domain corresponds to amino acid positions Y1 through V46 of SEQ ID NO:3, and to amino acid positions Y47 through V92 of SEQ ID NO:2. One skilled in the art also can employ conserved amino acid residues as guides to find corresponding amino acid residues between and among human and non-human sequences. For example, amino acid residues Q11 and P74 of SEQ ID NO:3 (human) correspond to R11 and Q74 of SEQ ID NO:14 (bovine). Corresponding positions also can be based on structural alignments, for example by using computer simulated alignments of protein structure. In other instances, corresponding regions can be identified.

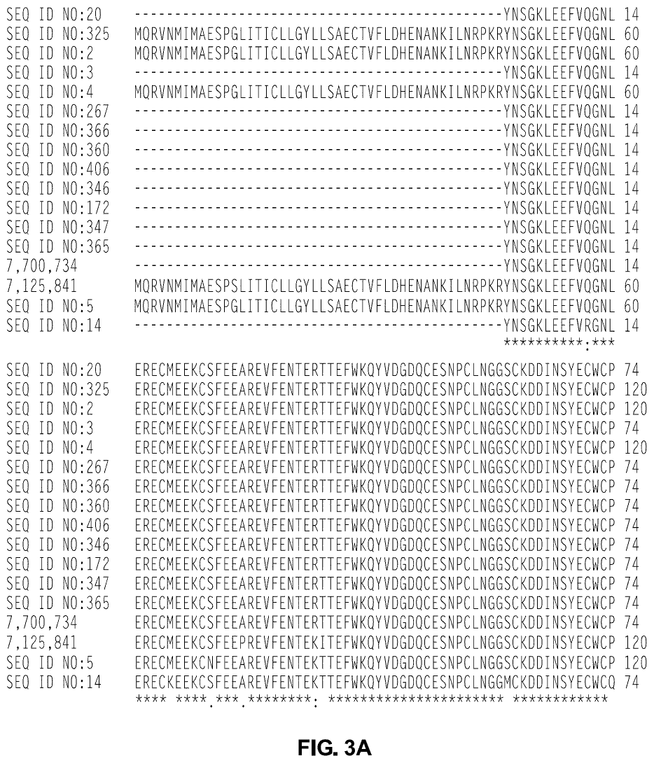

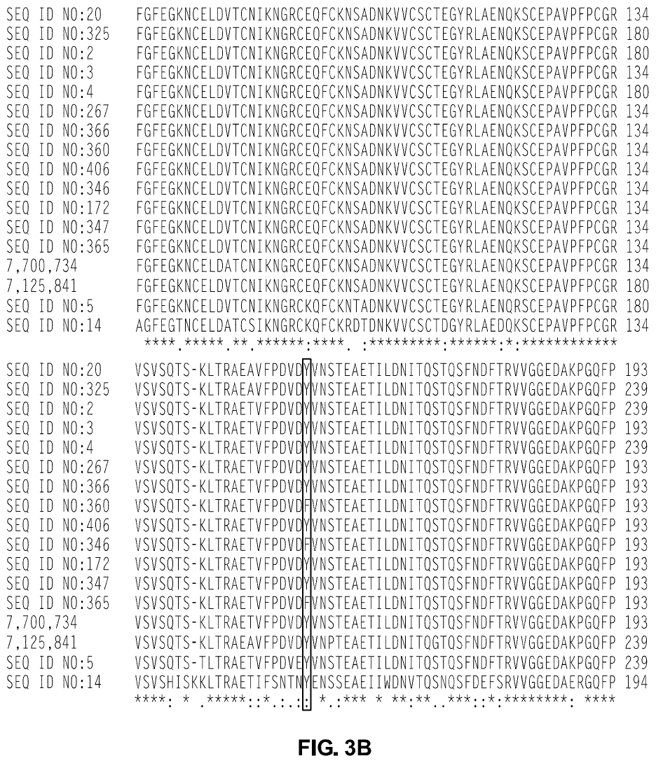

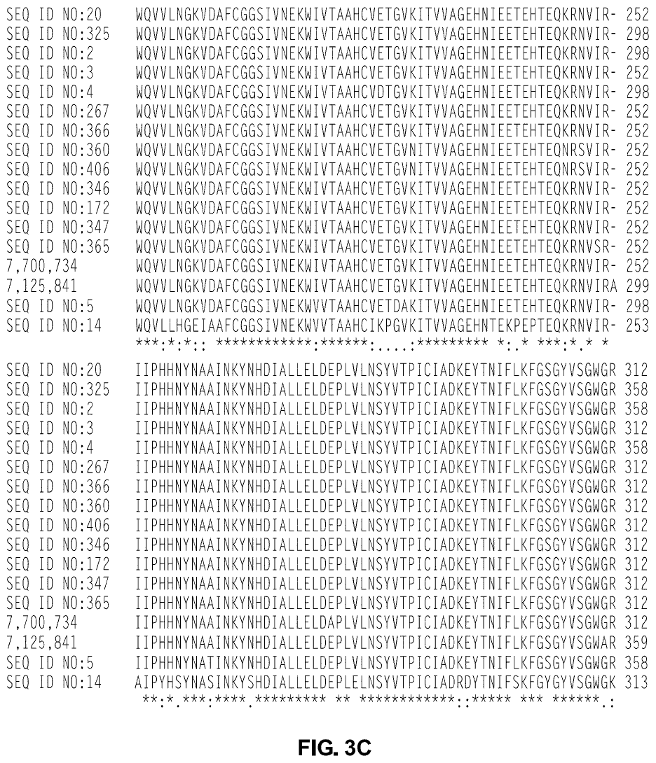

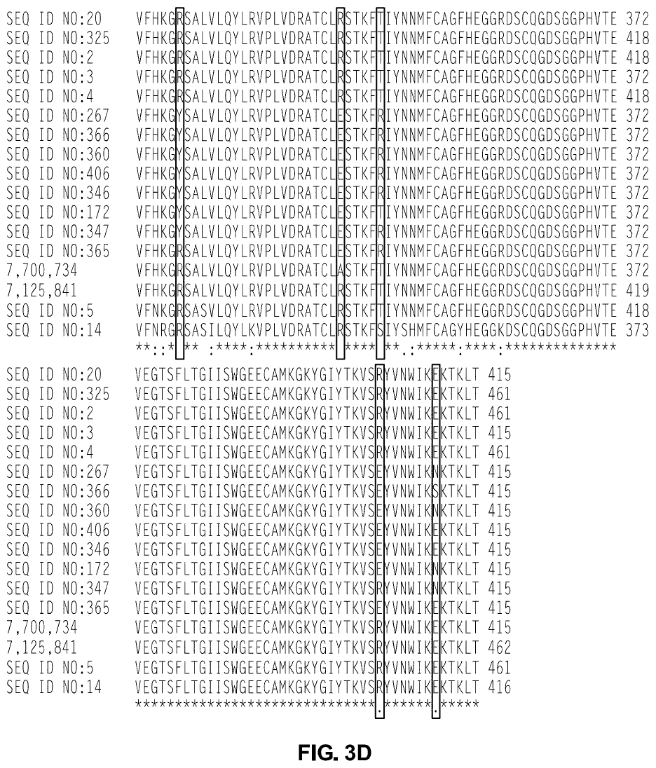

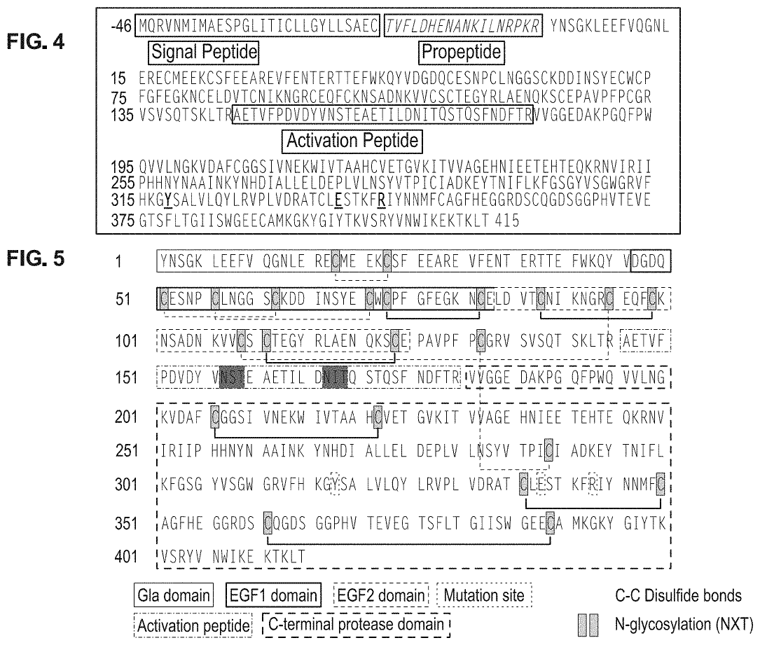

As used herein, the same, with reference to an amino acid replacement, refers to the identical replacement at the reference amino acid position in SEQ ID NO:3 in a corresponding position in another Factor IX polypeptide. For example, the same replacement with reference to the replacement of tyrosine at amino acid residue R318 in SEQ ID NO:3 is the replacement of tyrosine at amino acid residue R319 in SEQ ID NO:20 (see, for example, FIG. 3D). For example, the same replacement with reference to the replacement of asparagine at amino acid residue E410 in SEQ ID NO:3 is the replacement of asparagine at amino acid residue 5410 in SEQ ID NO:366. It is understood that reference to replacement of the same amino acid refers to replacement of amino acid residues that differ at the corresponding position from the replaced residue.

As used herein, a “proregion,” “propeptide,” or “pro sequence,” refers to a region or a segment that is cleaved to produce a mature protein. This can include segments that function to suppress proteolytic activity by masking the catalytic machinery and thus preventing formation of the catalytic intermediate (i.e., by sterically occluding the substrate binding site). A proregion is a sequence of amino acids positioned at the amino terminus of a mature biologically active polypeptide and can be as little as a few amino acids or can be a multi-domain structure.

As used herein, “mature factor IX” refers to a FIX polypeptide that lacks a signal sequence and a propeptide sequence. Typically, a signal sequence targets a protein for secretion via the endoplasmic reticulum (ER)-golgi pathway and is cleaved following insertion into the ER during translation. A propeptide sequence typically functions in post-translational modification of the protein and is cleaved prior to secretion of the protein from the cell. Thus, a mature FIX polypeptide is typically a secreted protein. In one example, a mature human FIX polypeptide is set forth in SEQ ID NO:3. The amino acid sequence set forth in SEQ ID NO:3 differs from that of the precursor polypeptide set forth in SEQ ID NO:2 in that SEQ ID NO:3 is lacking the signal sequence, which corresponds to amino acid residues 1-28 of SEQ ID NO:2, and also lacks the propeptide sequence, which corresponds to amino acid residues 29-46 of SEQ ID NO:2. Reference to a mature FIX polypeptide encompasses the single-chain zymogen form and the two-chain form. Thus, reference to a mature FIX polypeptide also refers to the two chain form containing the heavy chain and light chain (without the activation peptide corresponding to amino acids 192-226 of SEQ ID NO:2) joined by disulfide bonds.

As used herein, “wild-type” or “native” with reference to FIX refers to a FIX polypeptide encoded by a native or naturally occurring FIX gene, including allelic variants, that is present in an organism, including a human and other animals, in nature. Reference to wild-type factor IX without reference to a species is intended to encompass any species of a wild-type factor IX. Included among wild-type FIX polypeptides are the encoded precursor polypeptide, fragments thereof, and processed forms thereof, such as a mature form lacking the signal peptide as well as any pre- or post-translationally processed or modified forms thereof. Also included among native FIX polypeptides are those that are post-translationally modified, including, but not limited to, modification by glycosylation, carboxylation and hydroxylation. Native FIX polypeptides also include single-chain and two-chain forms. For example, humans express native FIX. The amino acid sequence of exemplary wild-type human FIX are set forth in SEQ ID NOS: 2 and 3, and allelic variants thereof. Other animals produce native FIX, including, but not limited to, chimpanzee (Pan troglodytes, SEQ ID NO:4), rhesus macaque (Macaca mulatta, SEQ ID NO:5), mouse (Mus musculus, SEQ ID NO:6), rat (Rattus norvegicus, SEQ ID NO:7), Guinea pig (Cavia porcellus, SEQ ID NO:8), pig (Sus scrofa, SEQ ID NO:9), dog (Canis familiaris, SEQ ID NO:10), cat (Felis catus, SEQ ID NO:11), rabbit (Oryctolagus cuniculus, SEQ ID NO:12), chicken (Gallus gallus, SEQ ID NO:13), cow (Bos Taurus, SEQ ID NO:14), sheep (Ovis aries, SEQ ID NO:15), frog (Xenopus tropicalis, SEQ ID NO:16), zebrafish (Danio rerio, SEQ ID NO:17), and Japanese pufferfish (Takifugu rubripes, SEQ ID NO:18).

As used herein, species variants refer to variants in polypeptides among different species, including different mammalian species, such as mouse and human.

As used herein, allelic variants refer to variations in proteins among members of the same species.

As used herein, a splice variant refers to a variant produced by differential processing of a primary transcript of genomic DNA that results in more than one type of mRNA.

As used herein, a zymogen refers to a protease that is activated by proteolytic cleavage, including maturation cleavage, such as activation cleavage, and/or complex formation with other protein(s) and/or cofactor(s). A zymogen is an inactive precursor of a proteolytic enzyme. Such precursors are generally larger, although not necessarily larger, than the active form. With reference to serine proteases, zymogens are converted to active enzymes by specific cleavage, including catalytic and autocatalytic cleavage, or by binding of an activating co-factor, which generates an active enzyme. For example, generally, zymogens are present in a single-chain form. Zymogens, generally, are inactive and can be converted to mature active polypeptides by catalytic or autocatalytic cleavage at one or more proteolytic sites to generate a multi-chain, such as a two-chain, polypeptide. A zymogen, thus, is an enzymatically inactive protein that is converted to a proteolytic enzyme by the action of an activator. Cleavage can be effected by auto activation. A number of coagulation proteins are zymogens; they are inactive, but become cleaved and activated upon the initiation of the coagulation system following vascular damage. With reference to FIX, the FIX polypeptides exist in the blood plasma as zymogens until cleavage by proteases, such as for example, activated FXI (FXIa) or FVIIa (in association with TF) to produce the two-chain form of FIX (FIXa).

As used herein, a capsid that transduces hepatocytes at a high level is one that transduces hepatocytes at a level at least as high as AAV8 capsid or an AAV with the DJ/8 (SEQ ID NO:427) capsid. In some embodiments, the capsid also transduces human and mouse hepatocytes at comparable or similar levels. Exemplary of these capsids are those designed KP1, KP2, and KP3 (SEQ ID NOs: 418-423).

As used herein, an activation sequence refers to a sequence of amino acids in a zymogen that is the site required for activation cleavage or maturation cleavage to form an active protease. Cleavage of an activation sequence can be catalyzed autocatalytically or by activating partners.

As used herein, activation cleavage is a type of maturation cleavage, which induces a conformation change that is required for the development of full enzymatic activity. This is a classical activation pathway, for example, for serine proteases in which a cleavage generates a new N-terminus that interacts with the conserved regions of the protease, such as Asp194 in chymotrypsin, to induce conformational changes required for activity. Activation can result in production of multi-chain forms of the proteases. In some instances, single chain forms of the protease can exhibit proteolytic activity.

As used herein, “activated Factor IX” or “FIXa” refers to any two-chain form of a FIXa polypeptide. A two-chain form typically results from proteolytic cleavage, but can be produced synthetically. Activated Factor IX, thus, includes the zymogen-like two-chain form with low coagulant activity, a fully activated form that occurs upon binding to FVIIIa and FX, and mutated forms that exist in a fully activated two-chain form or undergo conformational change to a fully activated form. For example, a single-chain form of FIX polypeptide (see, e.g., SEQ ID NO:3) is proteolytically cleaved after amino acid residues R145 and R180 of the mature FIX polypeptide. The cleavage products, FIX heavy chain and FIX light chain, which are held together by a disulfide bond (between amino acid residues 132C and 289C in the FIX of SEQ ID NO:3), form the two-chain activated FIX enzyme. Proteolytic cleavage can be carried out, for example, by activated Factor XIa (FXIa), and activated Factor VIIa (FVIIa) in complex with TF.

As used herein, a “property” of a FIX polypeptide refers to a physical or structural property, such three-dimensional structure, pI, half-life, conformation and other such physical characteristics.

As used herein, an “activity” of a FIX polypeptide refers to any activity exhibited by a factor IX polypeptide. Such activities can be tested in vitro and/or in vivo and include, but are not limited to, coagulation or coagulant activity, pro-coagulant activity, proteolytic or catalytic activity such as to effect factor X (FX) activation; antigenicity (ability to bind to or compete with a polypeptide for binding to an anti-FIX antibody); ability to bind factor VIIIa or factor X; and/or ability to bind to phospholipids. Activity can be assessed in vitro or in vivo using recognized assays, for example, by measuring coagulation in vitro or in vivo. The results of such assays indicate that a polypeptide exhibits an activity that can be correlated to activity of the polypeptide in vivo, in which in vivo activity can be referred to as biological activity. Assays to determine functionality or activity of modified forms of FIX are known to those of skill in the art. Exemplary assays to assess the activity of a FIX polypeptide include prothromboplastin time (PT) assay or the activated partial thromboplastin time (aPTT) assay to assess coagulant activity, or chromogenic assays using synthetic substrates to assess catalytic or proteolytic activity.

As used herein, “exhibits at least one activity” or “retains at least one activity” refers to the activity exhibited by a modified FIX polypeptide as compared to an unmodified FIX polypeptide of the same form and under the same conditions. For example, a modified FIX polypeptide in a two-chain form is compared with an unmodified FIX polypeptide in a two-chain form, under the same experimental conditions, where the only difference between the two polypeptides is the modification under study. In another example, a modified FIX polypeptide in a single-chain form is compared with an unmodified FIX polypeptide in a single-chain form, under the same experimental conditions, where the only difference between the two polypeptides is the modification under study. Typically, a modified FIX polypeptide that retains or exhibits at least one activity of an unmodified FIX polypeptide of the same form retains a sufficient amount of the activity such that, when administered in vivo, the modified FIX polypeptide is therapeutically effective as a procoagulant therapeutic. Generally, for a modified FIX polypeptide to retain therapeutic efficacy as a procoagulant, the amount of activity that is retained is or is about 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, 100%, 200%, 300%, 400%, 500%, or more, of the activity of an unmodified FIX polypeptide of the same form that displays therapeutic efficacy as a procoagulant. The amount of activity that is required to maintain therapeutic efficacy as a procoagulant can be empirically determined, if necessary. Typically, retention of 0.5% to 20%, 0.5% to 10%, or 0.5% to 5% of an activity is sufficient to retain therapeutic efficacy as a procoagulant in vivo.

It is understood that the activity being exhibited or retained by a modified FIX polypeptide can be any activity, including, but not limited to, coagulation or coagulant activity; pro-coagulant activity; proteolytic or catalytic activity such as to effect factor X (FX) activation; antigenicity (ability to bind to or compete with a polypeptide for binding to an anti-FIX antibody); ability to bind Factor VIIIa or Factor X; and/or ability to bind to phospholipids. In some instances, a modified FIX polypeptide can retain an activity that is increased compared to an unmodified FIX polypeptide. In some cases, a modified FIX polypeptide can retain an activity that is decreased compared to an unmodified FIX polypeptide. Activity of a modified FIX polypeptide can be any level of percentage of activity of the unmodified polypeptide, where both polypeptides are in the same form, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more, activity compared to the polypeptide that does not contain the modification at issue. For example, a modified FIX polypeptide can exhibit increased or decreased activity compared to the unmodified FIX polypeptide in the same form. For example, it can retain at least about or 1%, 2%, 3%, 4%, 5%, 6%, 7%, 8%, 9%, 10%, 20%, 30%, 40%, 50%, 60%, 70%, 80%, 90%, 95%, 96%, 97%, 98%, or at least 99%, of the activity of the unmodified FIX polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times, greater than unmodified FIX. The particular level to be retained is a function of the intended use of the polypeptide and can be empirically determined. Activity can be measured, for example, using in vitro or in vivo assays, such as those described herein.

As used herein, “coagulation activity” or “coagulant activity” or “pro-coagulant activity” refers to the ability of a polypeptide to effect coagulation. Assays to assess coagulant activity are known to those of skill in the art, and include prothrombin time (PT) assay or the activated partial thromboplastin time (aPTT) assay.

As used herein, the partial thromboplastin time (PTT) or activated partial thromboplastin time (aPTT or APTT) is a medical test that characterizes blood coagulation. Partial thromboplastin time (PTT) measures the overall speed at which blood clots by means of two consecutive series of biochemical reactions known as the “intrinsic” (also referred to as the contact activation pathway) and common coagulation pathways. The partial thromboplastin time (PTT) can be used with another measure of how quickly blood clotting takes place called the prothrombin time (PT), which measures the speed of clotting by means of the extrinsic pathway (also known as the tissue factor pathway). Normal PTT times require the presence of the coagulation Factors: I, II, V, VIII, IX, X, XI and XII. Deficiencies in factors VII or XIII are detected with the PTT test. This assay is exemplified in the Examples.

As used herein, “catalytic activity” or “proteolytic activity” with reference to FIX refers to the ability of a FIX protein to catalyze the proteolytic cleavage of a substrate, and are used interchangeably. Assays to assess such activities are known in the art. For example, the proteolytic activity of FIX can be measured using chromogenic substrates such as Mes-D-CHD-Gly-Arg-AMC, where cleavage of the substrate is monitored by absorbance and the rate of substrate hydrolysis determined by linear regression.

As used herein, domain (typically a sequence of three or more, generally 5 or 7 or more amino acids) refers to a portion of a molecule, such as proteins or the encoding nucleic acids, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as proteolytic activity. A protein can have one, or more than one, distinct domains. For example, a domain can be identified, defined or distinguished by homology of the sequence therein to related family members, such as homology to motifs that define a protease domain or a Gla domain. In another example, a domain can be distinguished by its function, such as by proteolytic activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains.

As used herein, a protease domain is the catalytically active portion of a protease. Reference to a protease domain of a protease includes the single, two- and multi-chain forms of any of these proteins. A protease domain of a protein contains all of the requisite properties of that protein required for its proteolytic activity, such as for example, the catalytic center. In reference to FIX, the protease domain shares homology and structural feature with the chymotrypsin/trypsin family protease domains, including the catalytic triad. For example, in the mature FIX polypeptide set forth in SEQ ID NO:3, the protease domain corresponds to amino acid positions 181 to 412.

As used herein, a gamma-carboxyglutamate (Gla) domain refers to the portion of a protein, for example a vitamin K-dependent protein, that contains post-translational modifications of glutamate residues, generally most, but not all of the glutamate residues, by vitamin K-dependent carboxylation to form Gla. The Gla domain is responsible for the high-affinity binding of calcium ions and binding to negatively-charged phospholipids. Typically, the Gla domain starts at the N-terminal extremity of the mature form of vitamin K-dependent proteins and ends with a conserved aromatic residue. In a mature FIX polypeptide the Gla domain corresponds to amino acid positions 1 to 46 of the exemplary polypeptide set forth in SEQ ID NO:3. Gla domains are well known and their locus can be identified in particular polypeptides. The Gla domains of the various vitamin K-dependent proteins share sequence, structural and functional homology, including the clustering of N-terminal hydrophobic residues into a hydrophobic patch that mediates interaction with negatively charged phospholipids on the cell surface membrane. Exemplary other Gla-containing polypeptides include, but are not limited to, FVII, FX, prothrombin, protein C, protein S, osteocalcin, matrix Gla protein, Growth-arrest-specific protein 6 (Gash), and protein Z.

As used herein, an epidermal growth factor (EGF) domain (EGF-1 or EGF-2) refers to the portion of a protein that shares sequence homology to a specific 30 to 40 amino acid portion of the epidermal growth factor (EGF) sequence. The EGF domain includes six cysteine residues that have been shown (in EGF) to be involved in disulfide bonds. The main structure of an EGF domain is a two-stranded beta-sheet followed by a loop to a C-terminal short two-stranded sheet. FIX contains two EGF domains: EGF-1 and EGF-2. These domains correspond to amino acid positions 47-83, and 84-125, respectively, of the mature FIX polypeptide set forth in SEQ ID NO:3.

As used herein, “unmodified polypeptide” or “unmodified FIX” and grammatical variations thereof refer to a starting polypeptide that is selected for modification as provided herein. The starting polypeptide can be a naturally-occurring, wild-type form of a polypeptide. In addition, the starting polypeptide can be altered or mutated, such that it differs from a native wild type isoform but is nonetheless referred to herein as a starting unmodified polypeptide relative to the subsequently modified polypeptides produced herein. Thus, existing proteins known in the art that have been modified to have a desired increase or decrease in a particular activity or property compared to an unmodified reference protein can be selected and used as the starting unmodified polypeptide. For example, a protein that has been modified from its native form by one or more single amino acid changes and possesses either an increase or decrease in a desired property, such as a change in an amino acid residue or residues to alter glycosylation, can be a target protein, referred to herein as unmodified, for further modification of either the same or a different property. Exemplary modified FIX polypeptides known in the art include any FIX polypeptide described in, for example, Schuettrumpf et al., (2005) Blood 105(6):2316-23; Melton et al., (2001) Blood Coagul. Fibrinolysis 12(4):237-43; Cheung et al., (1992) J. Biol. Chem. 267:20529-20531; Cheung et al., (1996) Proc. Natl. Acad. Sci. U.S.A. 93:11068-11073; Hopfner et al., (1997) EMBO J. 16:6626-6635; Sichler et al., (2003) J. Biol. Chem. 278:4121-4126; Begbie et al., (2005) J. Thromb. Haemost. 94(6):1138-47; Chang, J. et al., (1998) J. Biol. Chem. 273(20):12089-94; Yang, L. et al., (2002) J. Biol. Chem. 277(52):50756-60; Yang, L. et al., (2003) J. Biol. Chem. 278(27):25032-8; U.S. Pat. Nos. 5,969,040, 5,621,039, 6,423,826, 7,125,841, 6,017,882, 6,531,298; U.S. Patent Publication Nos. 2003/0211094, 2007/0254840, 2008/0188414, 2008/000422, 2008/0050772, 2008/0146494, 2008/0050772, 2008/0187955, 2004/0254106, 2005/0147618, 2008/0280818, 2008/0102115, 2008/0167219 and 2008/0214461; and International patent Application Publication Nos. WO 2007/112005, WO 2007/135182, WO 2008/082613, WO 2008/119815, WO 2008/119815, WO 2007/149406, WO 2007/112005 and WO 2004/101740.

As used herein, “modified factor IX polypeptides” and “modified factor IX” refer to a FIX polypeptide that has one or more amino acid differences compared to an unmodified factor IX polypeptide. The one or more amino acid differences can be amino acid mutations, such as one or more amino acid replacements (substitutions), insertions or deletions, or can be insertions or deletions of entire domains, and any combinations thereof. Typically, a modified FIX polypeptide has one or more modifications in the primary sequence compared to an unmodified FIX polypeptide. For example, a modified FIX polypeptide provided herein can have 1, 2, 3, 4, 5, 6, 7, 8, 9, 10, 11, 12, 13, 14, 15, 16, 17, 18, 19, 20, 30, 40, 50 or more amino acid differences compared to an unmodified FIX polypeptide. Any modification is contemplated as long as the resulting polypeptide exhibits at least one FIX activity associated with a native FIX polypeptide, such as, for example, catalytic activity, proteolytic activity, the ability to bind FVIIIa or the ability to bind phospholipids.

As used herein, “antithrombin III” or “AT-III” is a serine protease inhibitor (serpin). AT-III is synthesized as a precursor protein containing 464 amino acid residues (SEQ ID NO:21) that is cleaved during secretion to release a 432 amino acid mature antithrombin (SEQ ID NO:22).

As used herein, “heparin” refers to a heterogeneous group of straight-chain highly sulfated glycosaminoglycans having anticoagulant properties. Heparin can bind to AT-III to form the AT-III/heparin complex.

As used herein, “increased resistance to AT-III and/or heparin” refers to any amount of decreased sensitivity of a polypeptide, such as a modified FIX polypeptide, to the inhibitory effects of AT-III alone, heparin alone and/or the AT-III/heparin complex compared with a reference polypeptide, such as an unmodified FIX polypeptide. Increased resistance to AT-III, heparin, and/or an AT-III/heparin complex can be assayed by assessing the binding of a modified FIX polypeptide to AT-III, heparin, and/or an AT-III complex. Increased resistance also can be assayed by measuring inhibition of the catalytic or coagulant activity of a FIX polypeptide in the presence of AT-III, heparin, or an AT-III/heparin complex. Assays to determine the binding of a polypeptide to an inhibitor or the inhibition of enzymatic activity of a polypeptide by an inhibitor are known in the art. For covalent inhibitors, such as, for example, AT-III or an AT-III/heparin complex, a second order rate constant for inhibition can be measured. For non-covalent inhibitors, such as, for example, heparin, a kᵢ can be measured. In addition, surface plasma resonance, such as on a BIAcore biosensor instrument, also can be used to measure the binding of FIX polypeptides to AT-III, heparin, and/or an AT-III/heparin complex using one or more defined conditions. For covalent inhibitors such as AT-III or an AT-III/heparin complex, only an on-rate can be measured using BIAcore; for non-covalent inhibitors such as heparin, both the on-rate and off-rate can be measured. Assays to determine the inhibitory effect of, for example, AT-III/heparin on FIX coagulant activity also are known in the art. For example, the ability of a modified FIX polypeptide to cleave its substrate FX in the presence or absence of AT-III/heparin can be measured, and the degree to which AT-III/heparin inhibits the reaction determined. This can be compared to the ability of an unmodified FIX polypeptide to cleave its substrate FX in the presence or absence of AT-III. Alternatively, the second order rate constant for inhibition of a FIX polypeptide can be measured and compared to the second order rate constant for inhibition of an unmodified FIX polypeptide. When comparing second order rate constants for inhibition, increased resistance to inhibition means a decreased second order rate constant of inhibition. A modified polypeptide that exhibits increased resistance to AT-III and/or heparin exhibits, for example, an increase of 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more resistance to the effects of AT-III, heparin, and/or an AT-III/heparin complex, respectively, compared to an unmodified polypeptide.

As used herein, cofactors refer to proteins or molecules that bind to other specific proteins or molecules to form an active complex. In some examples, binding to a cofactor is required for optimal proteolytic activity. For example, FVIIIa is a cofactor of FIXa. Binding of FVIIIa to FIXa induces conformational changes that result in increased proteolytic activity of FIXa for its substrate, FX.

As used herein, a glycosylation site refers to an amino position in a polypeptide to which a carbohydrate moiety can be attached. Typically, a glycosylated protein contains one or more amino acid residues, such as asparagine or serine, for the attachment of the carbohydrate moieties.

As used herein, a native glycosylation site refers to the position of an amino acid to which a carbohydrate moiety is attached in a wild-type polypeptide. There are six native glycosylation sites in FIX; two N-glycosylation sites at N157 and N167, and six O-glycosylation sites at S53, S61, T159, T169, T172 and T179, corresponding to amino acid positions in the mature FIX polypeptide set forth in SEQ ID NO:3.

As used herein, a non-native glycosylation site refers to the position of an amino acid to which a carbohydrate moiety is attached in a modified polypeptide that is not present in a wild-type polypeptide. Non-native glycosylation sites can be introduced into a FIX polypeptide by amino acid replacement. O-glycosylation sites can be created, for example, by amino acid replacement of a native residue with a serine or threonine. N-glycosylation sites can be created, for example, by establishing the motif Asn-Xaa-Ser/Thr/Cys, where Xaa is not proline. Creation of this consensus sequence by amino acid modification can involve, for example, a single amino acid replacement of a native amino acid residue with an asparagine, a single amino acid replacement of a native amino acid residue with a serine, threonine or cysteine, or a double amino acid replacement involving a first amino acid replacement of a native residue with an asparagine and a second amino acid replacement of native residue with a serine, threonine or cysteine.

As used herein, “increased levels of glycosylation” and any grammatical variations thereof, refers to an increased amount of carbohydrate linked to a polypeptide as compared with a reference polypeptide or protein. The carbohydrate can be N-linked, O-linked, C-linked or be attached by any other linkage. The level of glycosylation can be increased by at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the level of glycosylation of an unmodified polypeptide. Assays to determine the level of glycosylation (i.e. amount of carbohydrate) of a polypeptide are known in the art. For example, the carbohydrate content or level of glycosylation can be assessed by high pH anion exchange chromatography, fluorophore assisted carbohydrate electrophoresis (FACE), sequential exoglycosidase digestions, mass spectrometry, NMR, gel electrophoresis, or any other method described herein or known in the art.

As used herein, “biological activity” refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a FIX polypeptide encompasses the coagulant activity.

As used herein, a pharmacokinetic property refers to a property related to the action of a drug or agent, such as a FIX polypeptide, in the body and in particular the rate at which drugs are absorbed, distributed, metabolized, and eliminated by the body. Pharmacokinetics can be assessed by various parameters. These include, but are not limited to, clearance, volume of distribution, in vivo recovery, total modified FIX polypeptide exposure in vivo, serum half-life, and mean resonance time (MRT). Pharmacokinetic properties of polypeptide can be assessed using methods well known in the art, such as, for example, administering the polypeptide to a human or animal model and assessing the amount of FIX in the body at various time points. The various parameters, such as clearance, volume of distribution, in vivo recovery, total modified FIX polypeptide exposure in vivo, serum half-life, and mean resonance time (MRT), are assessed using calculations well known in the art and described herein.

As used herein, “improved pharmacokinetic properties” refers to a desirable change in a pharmacokinetic property of a polypeptide, such as a modified FIX polypeptide, compared to, for example, an unmodified FIX polypeptide. The change can be an increase or a decrease.

As used herein, clearance refers to the removal of an agent, such as a polypeptide, from the body of a subject following administration. Clearance can be assessed using methods well known in the art, such as those described in Example 6. For example, assays in which a FIX polypeptide is administered to mice can be performed, and the clearance of the polypeptide from the body assessed by measuring the amount of FIX in the plasma at various time points and calculating the clearance as Dose/AUC_{0-inf}. Improved clearance of a modified FIX polypeptide compared to an unmodified FIX polypeptide refers to a decrease in clearance of a modified FIX polypeptide compared to an unmodified FIX polypeptide. The clearance of a modified FIX polypeptide can be decreased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99%, compared to an unmodified FIX polypeptide.

As used herein, mean resonance time (MRT) refers to the amount of time a FIX polypeptide resides in the body following administration. MRT can be assessed using methods well known in the art, such as those described in Example 6. For example, assays in which a FIX polypeptide is administered to mice can be performed, and the MRT of the polypeptide assessed by measuring the amount of FIX in the plasma at various time points and calculating the MRT as AUMC₀₋ₗₐₛₜ/AUC₀₋ₗₐₛₜ, where AUC₀₋ₗₐₛₜ is total area under the curve and AUMC₀₋ₗₐₛₜ is the total area under the first moment-versus-time curve. Improved MRT of a modified FIX polypeptide compared to an unmodified FIX polypeptide refers to an increase in MRT of a modified FIX polypeptide compared to an unmodified FIX polypeptide. The MRT of a modified FIX polypeptide can be increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500% or more compared to an unmodified FIX polypeptide.

As used herein, in vivo recovery refers to the percentage of FIX polypeptide detectable in the circulation after a period of time following administration in relation to the total amount of FIX polypeptide administered. In vivo recovery can be assessed using methods well known in the art, such as those described in Example 6. For example, assays in which a FIX polypeptide is administered to mice can be performed, and the in vivo recovery of the polypeptide assessed by measuring the amount of FIX in the plasma at Cₘₐₓ and comparing it to the amount of FIX administered. Improved in vivo recovery of a modified FIX polypeptide compared to an unmodified FIX polypeptide refers to an increase in in vivo recovery of a modified FIX polypeptide compared to an unmodified FIX polypeptide. The in vivo recovery of a modified FIX polypeptide can be increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500% or more compared to an unmodified FIX polypeptide.

As used herein, plasma half-life (t_{1/2}) refers the elimination half-life of a FIX polypeptide, or the time at which the plasma concentration of the FIX polypeptide has reached one half of its initial or maximal concentration following administration. Reference to plasma half-life includes plasma half-life during the α-, β-, and/or γ-phase. Plasma half-life can be assessed using methods well known in the art, such as those described in Example 6. For example, assays in which a FIX polypeptide is administered to mice can be performed, and the plasma half-life of the polypeptide assessed by measuring the amount of FIX in the plasma at various time points. The t_{1/2β}, for example, is calculated as −ln 2 divided by the negative slope during the terminal phase of the log-linear plot of the plasma FIX concentration-versus-time curve. Improved plasma half-life of a modified FIX polypeptide compared to an unmodified FIX polypeptide refers to an increase in plasma half-life of a modified FIX polypeptide compared to an unmodified FIX polypeptide. The plasma half-life of a modified FIX polypeptide can be increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500% or more compared to an unmodified FIX polypeptide.

As used herein, exposure in vivo refers to the amount of FIX polypeptide in the circulation following administration in relation to the plasma area under the concentration-time curve, or AUC, of FIX polypeptide administered. Exposure in vivo can be assessed using methods well known in the art, such as those described in Example 6. For example, assays in which a FIX polypeptide is administered to mice can be performed, and the in vivo recovery of the polypeptide assessed by measuring the amount of FIX in the plasma at various time points (i.e., AUC) and comparing it to the amount of FIX administered. Improved exposure in vivo of a modified FIX polypeptide compared to an unmodified FIX polypeptide refers to an increase in exposure in vivo of a modified FIX polypeptide compared to an unmodified FIX polypeptide. The exposure in vivo of a modified FIX polypeptide can be increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500% or more compared to an unmodified FIX polypeptide.

As used herein, volume of distribution refers to the distribution of a FIX polypeptide between plasma and the rest of the body following administration. It is defined as the volume in which the amount of polypeptide would need to be uniformly distributed to produce the observed concentration of polypeptide in the plasma. Volume of distribution can be assessed using methods well known in the art, such as those described in Example 6. For example, Vₛₛ, which is the steady state volume of distribution (calculated as MRT*Cl) and V_{z}, which is the volume of distribution based on the terminal elimination constant (β) (calculated as Cl/(ln 2/T_{1/2β}), can be assessed in assays in which a FIX polypeptide is administered to mice, and the concentration of the FIX in the plasma is determined at various time points. Improved volume of distribution of a modified FIX polypeptide compared with an unmodified FIX polypeptide, depending on the protein's mechanism of clearance and safety profile, can refer to either an increase or a decrease in the volume of distribution of a modified FIX polypeptide. For example, in cases where the polypeptide is distributed among multiple compartments, a decreased volume of distribution of a modified FIX polypeptide could result in significantly increased drug exposure and activity in the compartment of interest (e.g., the vascular compartment versus an extravascular compartment) compared with an unmodified FIX polypeptide. In other cases, for example, when drug safety is limited by Cₘₐₓ, redistribution into other compartments (e.g., binding to the surface of endothelial cells) can result in a longer terminal half-life and/or duration of action within the compartment of interest and a superior safety profile compared to the unmodified FIX polypeptide. The volume of distribution of a modified FIX polypeptide can be decreased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98% or 99% compared to an unmodified FIX polypeptide. In other examples, the volume of distribution of the modified FIX polypeptide is increased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500% or more of the volume of distribution of an unmodified FIX polypeptide.

As used herein, and known to those of skill in the art, International Units (IU) for coagulation factors, such as FIX and FVII, are assigned according to the World Health Organization (WHO) current International Standards (see, e.g., nibsc.org/documents/ifu/09-172.pdf). For example, for the modified FIX herein that comprises R318Y/R338E/T343R (SEQ ID NO: 394), 0.1 mg=460 IU. Similarly, other IUs for other coagulation factors, such as FVII, are defined by WHO. Hence, normal FIX levels are generally about or at or above 50 IU/dL, up to about 150 IU. IUs are defined by WHO International Standard 4th International Standard for Blood Coagulation Factors II, VII, IX, X, Plasma NIB SC code: 09/172 (Version 3.0, Dated 24 Feb. 2016). 100 IU/dl is 100% activity. Near normal coagulation FIX a FIX has about or at about 40%-150% of the activity in blood relative to the WHO 4th International Standard, where 100 IU/dl is 100% activity. Mild hemophilia is in the range of at or about 5 IU/dL-40 IU dL. The prophylactic methods herein either bring the range of FIX levels to mild hemophilia, or up to normal levels, and can achieve normal coagulation pharmacodynamics.

As used herein the term “assess”, and grammatical variations thereof, is intended to include quantitative and qualitative determination in the sense of obtaining an absolute value for the activity of a polypeptide, and also of obtaining an index, ratio, percentage, visual or other value indicative of the level of the activity. Assessment can be direct or indirect. For example, detection of cleavage of a substrate by a polypeptide can be by direct measurement of the product, or can be indirectly measured by determining the resulting activity of the cleaved substrate.

As used herein, “chymotrypsin numbering” refers to the amino acid numbering of a mature bovine chymotrypsin polypeptide of SEQ ID NO:19. Alignment of a protease domain of another protease, such as for example the protease domain of factor IX, can be made with chymotrypsin. In such an instance, the amino acids of factor IX that correspond to amino acids of chymotrypsin are given the numbering of the chymotrypsin amino acids. Corresponding positions can be determined by such alignment by one of skill in the art using manual alignments or by using the numerous alignment programs available (for example, BLASTP). Corresponding positions also can be based on structural alignments, for example by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm. The corresponding chymotrypsin numbers of amino acid positions 181 to 415 of the FIX polypeptide set forth in SEQ ID NO:3 are provided in Table 1. The amino acid positions relative to the sequence set forth in SEQ ID NO:3 are in normal font, the amino acid residues at those positions are in bold, and the corresponding chymotrypsin numbers are in italics. For example, upon alignment of the mature factor IX (SEQ ID NO:3) with mature chymotrypsin (SEQ ID NO:19), the valine (V) at amino acid position 181 in factor IX is given the chymotrypsin numbering of V16. Subsequent amino acids are numbered accordingly. In one example, a glutamic acid (E) at amino acid position 213 of the mature factor IX (SEQ ID NO:3) corresponds to amino acid position E49 based on chymotrypsin numbering. Where a residue exists in a protease, but is not present in chymotrypsin, the amino acid residue is given a letter notation. For example, A95a and A95b by chymotrypsin numbering correspond to A261 and A262, respectively, by numbering relative to the mature factor IX sequence (SEQ ID NO:3).

TABLE 1
Chymotrypsin numbering of factor IX

| 181 | 182 | 183 | 184 | 185 | 186 | 187 | 188 | 189 | 190 | 191 | 192 | 193 | 194 | 195 |
|---|---|---|---|---|---|---|---|---|---|---|---|---|---|---|
| V | V | G | G | E | D | A | K | P | G | Q | F | P | W | Q |
| 16 | 17 | 18 | 19 | 20 | 21 | 22 | 23 | 24 | 25 | 26 | 27 | 28 | 29 | 30 |
| 196 | 197 | 198 | 199 | 200 | 201 | 202 | 203 | 204 | 205 | 206 | 207 | 208 | 209 | 210 |
| V | V | L | N | G | K | V | D | A | F | C | G | G | S | I |
| 31 | 32 | 33 | 34 | 35 | 37 | 38 | 39 | 40 | 41 | 42 | 43 | 44 | 45 | 46 |
| 211 | 212 | 213 | 214 | 215 | 216 | 217 | 218 | 219 | 220 | 221 | 222 | 223 | 224 | 225 |
| V | N | E | K | W | I | V | T | A | A | H | C | V | E | T |
| 47 | 48 | 49 | 50 | 51 | 52 | 53 | 54 | 55 | 56 | 57 | 58 | 59 | 60 | 60A |
| 226 | 227 | 228 | 229 | 230 | 231 | 232 | 233 | 234 | 235 | 236 | 237 | 238 | 239 | 240 |
| G | V | K | I | T | V | V | A | G | E | H | N | I | E | E |
| 61 | 62 | 63 | 64 | 65 | 66 | 67 | 68 | 69 | 70 | 71 | 72 | 73 | 74 | 75 |
| 241 | 242 | 243 | 244 | 245 | 246 | 247 | 248 | 249 | 250 | 251 | 252 | 253 | 254 | 255 |
| T | E | H | T | E | Q | K | R | N | V | I | R | I | I | P |
| 76 | 77 | 78 | 79 | 80 | 81 | 82 | 83 | 84 | 85 | 86 | 87 | 88 | 89 | 90 |
| 256 | 257 | 258 | 259 | 260 | 261 | 262 | 263 | 264 | 265 | 266 | 267 | 268 | 269 | 270 |
| H | H | N | Y | N | A | A | I | N | K | Y | N | H | D | I |
| 91 | 92 | 93 | 94 | 95 | 95A | 95B | 96 | 97 | 98 | 99 | 100 | 101 | 102 | 103 |
| 271 | 272 | 273 | 274 | 275 | 276 | 277 | 278 | 279 | 280 | 281 | 282 | 283 | 284 | 285 |
| A | L | L | E | L | D | E | P | L | V | L | N | S | Y | V |
| 104 | 105 | 106 | 107 | 108 | 109 | 110 | 111 | 112 | 113 | 114 | 115 | 116 | 117 | 118 |
| 286 | 287 | 288 | 289 | 290 | 291 | 292 | 293 | 294 | 295 | 296 | 297 | 298 | 299 | 300 |
| T | P | I | C | I | A | D | K | E | Y | T | N | I | F | L |
| 119 | 120 | 121 | 122 | 123 | 124 | 125 | 126 | 127 | 128 | 129 | 129A | 129B | 130 | 131 |
| 301 | 302 | 303 | 304 | 305 | 306 | 307 | 308 | 309 | 310 | 311 | 312 | 313 | 314 | 315 |
| K | F | G | S | G | Y | V | S | G | W | G | R | V | F | H |
| 132 | 133 | 134 | 135 | 136 | 137 | 138 | 139 | 140 | 141 | 142 | 143 | 144 | 145 | 147 |
| 316 | 317 | 318 | 319 | 320 | 321 | 322 | 323 | 324 | 325 | 326 | 327 | 328 | 329 | 330 |
| K | G | R | S | A | L | V | L | Q | Y | L | R | V | P | L |
| 148 | 149 | 150 | 151 | 152 | 153 | 154 | 155 | 156 | 157 | 158 | 159 | 160 | 161 | 162 |
| 331 | 332 | 333 | 334 | 335 | 336 | 337 | 338 | 339 | 340 | 341 | 342 | 343 | 344 | 345 |
| V | D | R | A | T | C | L | R | S | T | K | F | T | I | Y |
| 163 | 164 | 165 | 166 | 167 | 168 | 169 | 170 | 171 | 172 | 173 | 174 | 175 | 176 | 177 |
| 346 | 347 | 348 | 349 | 350 | 351 | 352 | 353 | 354 | 355 | 356 | 357 | 358 | 359 | 360 |
| N | N | M | F | C | A | G | F | H | E | G | G | R | D | S |
| 178 | 179 | 180 | 181 | 182 | 183 | 184 | 184A | 185 | 186 | 187 | 188 | 188A | 189 | 190 |
| 361 | 362 | 363 | 364 | 365 | 366 | 367 | 368 | 369 | 370 | 371 | 372 | 373 | 374 | 375 |
| C | Q | G | D | S | G | G | P | H | V | T | E | V | E | G |
| 191 | 192 | 193 | 194 | 195 | 196 | 197 | 198 | 199 | 200 | 201 | 202 | 203 | 204 | 205 |
| 376 | 377 | 378 | 379 | 380 | 381 | 382 | 383 | 384 | 385 | 386 | 387 | 388 | 389 | 390 |
| T | S | F | L | T | G | I | I | S | W | G | E | E | C | A |
| 206 | 207 | 208 | 209 | 210 | 211 | 212 | 213 | 214 | 215 | 216 | 217 | 219 | 220 | 221 |
| 391 | 392 | 393 | 394 | 395 | 396 | 397 | 398 | 399 | 400 | 401 | 402 | 403 | 404 | 405 |
| M | K | G | K | Y | G | I | Y | T | K | V | S | R | Y | V |
| 221A | 222 | 223 | 224 | 225 | 226 | 227 | 228 | 229 | 230 | 231 | 232 | 233 | 234 | 235 |

| 406 | 407 | 408 | 409 | 410 | 411 | 412 | 413 | 414 | 415 |
|---|---|---|---|---|---|---|---|---|---|
| N | W | I | K | E | K | T | K | L | T |
| 236 | 237 | 328 | 239 | 240 | 241 | 242 | 243 | 244 | 245 |

As used herein, nucleic acids include DNA, RNA and analogs thereof, including peptide nucleic acids (PNAs), and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids that occur in the various sequences of amino acids provided herein are identified according to their known, three-letter or one-letter abbreviations (Table 3). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain). In keeping with standard polypeptide nomenclature described in J. Biol. Chem., 243:3557-3559 (1968), and adopted in 37 C.F.R. §§ 1.821-1.822, abbreviations for the amino acid residues are shown in Table 3:

TABLE 3
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 3) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, “non-natural amino acid” refers to an organic compound containing an amino group and a carboxylic acid group that is not one of the naturally-occurring amino acids listed in Table 3. Non-naturally occurring amino acids thus include, for example, amino acids or analogs of amino acids other than the 20 naturally-occurring amino acids and include, but are not limited to, the D-isostereomers of amino acids. Exemplary non-natural amino acids are known to those of skill in the art and can be included in a modified factor IX polypeptide.

For purposes herein, conservative amino acid substitutions may be made in any of polypeptides and domains thereof provided that the resulting protein exhibits an activity of a FIX. Conservative amino acid substitutions, such as those set forth in Table 4, are those that do not eliminate proteolytic activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Also included within the definition, is the catalytically active fragment of an MTSP, particularly a single chain protease portion. Conservative amino acid substitutions are made, for example, in accordance with those set forth in Table 4, which sets forth exemplary conservative amino acid substitutions, as follows:

TABLE 4

| Original residue | Conservative substitution |
|---|---|
| Ala (A) | Gly; Ser, Abu |
| Arg (R) | Lys, orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Met; Nle; Nva |
| Leu (L) | Ile; Val; Met; Nle; Nv |
| Lys (K) | Arg; Gln; Glu |
| Met (M) | Leu; Tyr; Ile; NLe Val |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met; Nle; Nv |

Other substitutions are also permissible and may be determined empirically or in accord with known conservative substitutions.

As used herein, a DNA construct is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule cannot be paired. Such unpaired ends will, in general, not exceed 20 nucleotides in length.

As used herein, “primary sequence” refers to the sequence of amino acid residues in a polypeptide.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, the terms “homology” and “identity” are used interchangeably, but homology for proteins can include conservative amino acid changes. In general to identify corresponding positions the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo et al. (1988) SIAM J. Applied Math 48:1073).

As use herein, “sequence identity” refers to the number of identical amino acids (or nucleotide bases) in a comparison between a test and a reference polypeptide or polynucleotide. Homologous polypeptides refer to a pre-determined number of identical or homologous amino acid residues. Homology includes conservative amino acid substitutions as well identical residues. Sequence identity can be determined by standard alignment algorithm programs used with default gap penalties established by each supplier. Homologous nucleic acid molecules refer to a pre-determined number of identical or homologous nucleotides. Homology includes substitutions that do not change the encoded amino acid (i.e., “silent substitutions”) as well identical residues. Substantially homologous nucleic acid molecules hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule. (For determination of homology of proteins, conservative amino acids can be aligned as well as identical amino acids; in this case, percentage of identity and percentage homology varies). Whether any two nucleic acid molecules have nucleotide sequences (or any two polypeptides have amino acid sequences) that are at least 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. U.S.A. 85:2444 (other programs include the GCG program package (Devereux, J., et al. (1984) Nucleic Acids Research 12(I):387), BLASTP, BLASTN, FASTA (Atschul, S. F., et al. (1990) J. Molec. Biol. 215:403; Guide to Huge Computers, Martin J. Bishop, ed., Academic Press, San Diego (1994), and Carillo et al. (1988) SIAM J. Applied Math 48:1073)). For example, the BLAST function of the National Center for Biotechnology Information database can be used to determine identity. Other commercially or publicly available programs include DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. J. Mol. Biol. 48:443 (1970), as revised by Smith and Waterman (Adv. Appl. Math. 2: 482 (1981)). Briefly, a GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” represents a comparison between a test and a reference polypeptide or polynucleotide. In one non-limiting example, “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, it also is understood that the terms “substantially identical” or “similar” varies with the context as understood by those skilled in the relevant art, but that those of skill can assess such.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “specifically hybridizes” refers to annealing, by complementary base-pairing, of a nucleic acid molecule (e.g., an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, isolated or purified polypeptide or protein or biologically-active portion thereof is substantially free of cellular material or other contaminating proteins from the cell of tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as proteolytic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

The term substantially free of cellular material includes preparations of proteins in which the protein is separated from cellular components of the cells from which it is isolated or recombinantly-produced. In one embodiment, the term substantially free of cellular material includes preparations of protease proteins having less that about 30% (by dry weight) of non-protease proteins (also referred to herein as a contaminating protein), generally less than about 20% of non-protease proteins or 10% of non-protease proteins or less that about 5% of non-protease proteins. When the protease protein or active portion thereof is recombinantly produced, it also is substantially free of culture medium, i.e., culture medium represents less than, about, or equal to 20%, 10% or 5% of the volume of the protease protein preparation.

As used herein, the term substantially free of chemical precursors or other chemicals includes preparations of protease proteins in which the protein is separated from chemical precursors or other chemicals that are involved in the synthesis of the protein. The term includes preparations of protease proteins having less than about 30% (by dry weight), 20%, 10%, 5% or less of chemical precursors or non-protease chemicals or components.

As used herein, production by recombinant methods by using recombinant DNA methods refers to the use of the well-known methods of molecular biology for expressing proteins encoded by cloned DNA.

As used herein, vector (or plasmid) refers to discrete elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as bacterial artificial chromosomes, yeast artificial chromosomes and mammalian artificial chromosomes. Selection and use of such vehicles are well known to those of skill in the art.

As used herein, expression refers to the process by which nucleic acid is transcribed into mRNA and translated into peptides, polypeptides, or proteins. If the nucleic acid is derived from genomic DNA, expression can, if an appropriate eukaryotic host cell or organism is selected, include processing, such as splicing of the mRNA.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, an adenovirus refers to any of a group of DNA-containing viruses that cause conjunctivitis and upper respiratory tract infections in humans.

As used herein, naked DNA refers to histone-free DNA that can be used for vaccines and gene therapy. Naked DNA is the genetic material that is passed from cell to cell during a gene transfer processed called transformation or transfection. In transformation or transfection, purified or naked DNA that is taken up by the recipient cell will give the recipient cell a new characteristic or phenotype.

As used herein, operably or operatively linked when referring to DNA segments means that the segments are arranged so that they function in concert for their intended purposes, e.g., transcription initiates in the promoter and proceeds through the coding segment to the terminator.

As used herein, an agent that modulates the activity of a protein or expression of a gene or nucleic acid either decreases or increases or otherwise alters the activity of the protein or, in some manner, up- or down-regulates or otherwise alters expression of the nucleic acid in a cell.

As used herein, a “chimeric protein” or “fusion protein” refers to a polypeptide operatively-linked to a different polypeptide. A chimeric or fusion protein provided herein can include one or more FIX polypeptides, or a portion thereof, and one or more other polypeptides for any one or more of a transcriptional/translational control signals, signal sequences, a tag for localization, a tag for purification, part of a domain of an immunoglobulin G, and/or a targeting agent. A chimeric FIX polypeptide also includes those having their endogenous domains or regions of the polypeptide exchanged with another polypeptide. These chimeric or fusion proteins include those produced by recombinant means as fusion proteins, those produced by chemical means, such as by chemical coupling, through for example, coupling to sulfhydryl groups, and those produced by any other method whereby at least one polypeptide (i.e., FIX), or a portion thereof, is linked, directly or indirectly via linker(s) to another polypeptide.

As used herein, operatively-linked when referring to a fusion protein refers to a protease polypeptide and a non-protease polypeptide that are fused in-frame to one another. The non-protease polypeptide can be fused to the N-terminus or C-terminus of the protease polypeptide.

As used herein, a targeting agent, is any moiety, such as a protein or effective portion thereof, that provides specific binding to a cell surface molecule, such a cell surface receptor, which in some instances can internalize a bound conjugate or portion thereof. A targeting agent also can be one that promotes or facilitates, for example, affinity isolation or purification of the conjugate; attachment of the conjugate to a surface; or detection of the conjugate or complexes containing the conjugate.

As used herein, derivative or analog of a molecule refers to a portion derived from or a modified version of the molecule.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are those involving coagulation, including those mediated by coagulation proteins and those in which coagulation proteins play a role in the etiology or pathology. Diseases and disorders also include those that are caused by the absence of a protein such as in hemophilia, and of particular interest herein are those disorders where coagulation does not occur due to a deficiency of defect in a coagulation protein.

As used herein, “procoagulant” refers to any substance that promotes blood coagulation.

As used herein, “anticoagulant” refers to any substance that inhibits blood coagulation.

As used herein, “hemophilia” refers to a bleeding disorder caused by a deficiency in a blood clotting factor. Hemophilia can be the result, for example, of absence, reduced expression, or reduced function of a clotting factor. The most common type of hemophilia is hemophilia A, which results from a deficiency in factor VIII. The second most common type of hemophilia is hemophilia B, which results from a deficiency in factor IX. Hemophilia C, also called FXI deficiency, is a milder and less common form of hemophilia.

As used herein, “congenital hemophilia” refers to types of hemophilia that are inherited. Congenital hemophilia results from mutation, deletion, insertion, or other modification of a clotting factor gene in which the production of the clotting factor is absent, reduced, or non-functional. For example, hereditary mutations in clotting factor genes, such as factor VIII and factor IX result in the congenital hemophilias, Hemophilia A and B, respectively.

As used herein, “acquired hemophilia” refers to a type of hemophilia that develops in adulthood from the production of autoantibodies that inactivate FVIII.

As used herein, “bleeding disorder” refers to a condition in which the subject has a decreased ability to control bleeding. Bleeding disorders can be inherited or acquired, and can result from, for example, defects or deficiencies in the coagulation pathway, defects or deficiencies in platelet activity, or vascular defects.

As used herein, “acquired bleeding disorder” refers to bleeding disorders that results from clotting deficiencies caused by conditions such as liver disease, vitamin K deficiency, or Coumadin® (warfarin) or other anti-coagulant therapy.

As used herein, “treating” a subject having a disease or condition means that a polypeptide, composition or other product provided herein is administered to the subject.

As used herein, a therapeutic agent, therapeutic regimen, radioprotectant, or chemotherapeutic mean conventional drugs and drug therapies, including vaccines, which are known to those skilled in the art. Radiotherapeutic agents are well known in the art.

As used herein, “treatment” means any manner in which the symptoms of a condition, disorder or disease are ameliorated or otherwise beneficially altered. Hence, treatment encompasses prophylaxis, therapy and/or cure. Treatment also encompasses any pharmaceutical use of the compositions herein. Treatment also encompasses any pharmaceutical use of a modified FIX and compositions provided herein.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “prevention” or “prophylaxis” refers to methods in which the risk of developing disease or condition is reduced. Prophylaxis includes reduction in the risk of developing a disease or condition and/or a prevention of worsening of symptoms or progression of a disease or reduction in the risk of worsening of symptoms or progression of a disease.

As used herein, an “effective amount” of a compound or composition for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Typically, repeated administration is required to achieve a desired amelioration of symptoms.

As used herein, “therapeutically effective amount” or “therapeutically effective dose” refers to an agent, compound, material, or composition containing a compound that is at least sufficient to produce a therapeutic effect. An effective amount is the quantity of a therapeutic agent necessary for preventing, curing, ameliorating, arresting or partially arresting a symptom of a disease or disorder.

As used herein, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

As used herein, a “combination” refers to any association between two or among more items. The association can be spatial or refer to the use of the two or more items for a common purpose.

As used herein, a “composition” refers to any mixture of two or more products or compounds (e.g., agents, modulators, regulators, etc.). It can be a solution, a suspension, liquid, powder, a paste, aqueous or non-aqueous formulations or any combination thereof.

As used herein, an “article of manufacture” is a product that is made and sold. As used throughout this application, the term is intended to encompass modified protease polypeptides and nucleic acids contained in articles of packaging.

As used herein, “fluid” refers to any composition that can flow. Fluids thus encompass compositions that are in the form of semi-solids, pastes, solutions, aqueous mixtures, gels, lotions, creams and other such compositions.

As used herein, a “kit” refers to a packaged combination, optionally including reagents and other products and/or components for practicing methods using the elements of the combination. For example, kits containing a modified protease polypeptide or nucleic acid molecule provided herein and another item for a purpose including, but not limited to, administration, diagnosis, and assessment of a biological activity or property are provided. Kits optionally include instructions for use.

As used herein, antibody includes antibody fragments, such as Fab fragments, which are composed of a light chain and the variable region of a heavy chain.

As used herein, a “receptor” refers to a molecule that has an affinity for a particular ligand. Receptors can be naturally-occurring or synthetic molecules. Receptors also can be referred to in the art as anti-ligands.

As used herein, “animal” includes any animal, such as, but not limited to; primates including humans, gorillas and monkeys; rodents, such as mice and rats; fowl, such as chickens; ruminants, such as goats, cows, deer, sheep; ovine, such as pigs and other animals. Non-human animals exclude humans as the contemplated animal. The proteases provided herein are from any source, animal, plant, prokaryotic and fungal.

As used herein, gene therapy involves the transfer of heterologous nucleic acid, such as DNA, into certain cells, target cells, of a mammal, particularly a human, with a disorder or condition for which such therapy is sought. The nucleic acid, such as DNA, is introduced into the selected target cells, such as directly or in a vector or other delivery vehicle, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy also can be used to deliver nucleic acid encoding a gene product that replaces a defective gene or supplements a gene product produced by the mammal or the cell in which it is introduced. The introduced nucleic acid can encode a therapeutic compound, such as a protease or modified protease, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, heterologous nucleic acid is nucleic acid that is not normally produced in vivo by the cell in which it is expressed or that is produced by the cell but is at a different locus or expressed differently or that mediates or encodes mediators that alter expression of endogenous nucleic acid, such as DNA, by affecting transcription, translation, or other regulatable biochemical processes. Heterologous nucleic acid is generally not endogenous to the cell into which it is introduced, but has been obtained from another cell or prepared synthetically. Heterologous nucleic acid can be endogenous, but is nucleic acid that is expressed from a different locus or altered in its expression. Generally, although not necessarily, such nucleic acid encodes RNA and proteins that are not normally produced by the cell or in the same way in the cell in which it is expressed. Heterologous nucleic acid, such as DNA, also can be referred to as foreign nucleic acid, such as DNA. Thus, heterologous nucleic acid or foreign nucleic acid includes a nucleic acid molecule not present in the exact orientation or position as the counterpart nucleic acid molecule, such as DNA, is found in a genome. It also can refer to a nucleic acid molecule from another organism or species (i.e., exogenous).

Any nucleic acid, such as DNA, that one of skill in the art would recognize or consider as heterologous or foreign to the cell in which the nucleic acid is expressed is herein encompassed by heterologous nucleic acid; heterologous nucleic acid includes exogenously added nucleic acid that also is expressed endogenously. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and nucleic acid, such as DNA, that encodes other types of proteins, such as antibodies. Antibodies that are encoded by heterologous nucleic acid can be secreted or expressed on the surface of the cell in which the heterologous nucleic acid has been introduced.

As used herein, a therapeutically effective product for gene therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease. Also included are biologically active nucleic acid molecules, such as RNAi and antisense.

As used herein, recitation that a polypeptide “consists essentially” of a recited sequence of amino acids means that only the recited portion, or a fragment thereof, of the full-length polypeptide is present. The polypeptide can optionally, and generally will, include additional amino acids from another source or can be inserted into another polypeptide.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. About also includes the exact amount. Hence, “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

Exemplary abbreviations as used below, include, but are not limited to:

### List of Abbreviations

AAV Adeno-associated viral

aPTT Activated partial thromboplastin time

ATIII Anti-thrombin III

AUC Area under curve

BA Bioavailability

CDC Centers for Disease Control and Prevention

CHO Chinese Hamster Ovary

CHO Chinese Hamster Ovary

CHPS Canadian hemophilia primary prophylaxis study

CHPS Canadian hemophilia primary prophylaxis study

CL Clearance

Cₘₐₓ Maximum concentration

C_{ss max} Maximum concentration at steady-state

Cₜ Concentration at time t

CVAD Central venous access devices

DNA Deoxyribonucleic acid

DP Drug product

ED Exposure days

EHL Extended half-life

EPAR European Public Assessment Report

FIX Factor IX

GLA Gamma-carboxyglutamic acid

HB Hemophilia B

HTC Hemophilia treatment centers

INN International Non-proprietary Name

IV Intravenously

IVR In vivo recovery

Kel terminal rate constant

MOA Mechanism of action

MRI Magnetic resonance imaging

MRT mean residence time;

ND Not Detected

NIB SC National Institute of Biological Standards and Control

NT Not Tested

PD Pharmacodynamics

PEG Polyethylene glycol

PK Pharmacokinetic

PND Prenatal diagnosis

PT Prothrombin time

PTC Plasma thromboplastin component

PUP Previously untreated patient

QD Once a day

RhFIX Recombinant human FIX

SC Subcutaneous

SNVs Single nucleotide variants

SWFI Sterile water for injection

TAT Thrombin-anti-thrombin

UDC Universal Data Collection

Vₛₛ volume of distribution at steady state

WBCT Whole Blood Clotting Time

WCB Working cell bank

WFH World Federation of Hemophilia's

WHO World Health Organization

WT Wild type

WT-FIX Wild type FIX

### B. HEMOSTASIS AND ROLE OF FACTOR IX

Provided herein are modified Factor IX (FIX) polypeptides, including modified FIX and FIXa polypeptides and catalytically active fragments thereof. Factor IX polypeptides play a role in the regulation of and process of hemostasis, and hence can be used as therapeutic agents. Effective delivery of therapeutic proteins such as FIX for clinical use is a major challenge to pharmaceutical science. Once in the blood stream, these proteins are constantly eliminated from circulation within a short time by different physiological processes, involving metabolism as well as clearance using normal pathways for protein elimination, such as (glomerular) filtration in the kidneys or proteolysis in blood. Once in the luminal gastrointestinal tract, these proteins are constantly digested by luminal proteases. The latter can be a limiting process affecting the half-life of proteins used as therapeutic agents in intravenous injection. Additionally, inhibitors in the blood can specifically inhibit the activity of the therapeutic protein. For example, antithrombin (AT-III), heparin, and the AT-III/heparin complex, can inhibit the coagulant activity of FIX. More efficacious variants of FIX with improved properties, including improved pharmacokinetic and pharmacodynamic properties, increased catalytic activity, and/or increased resistance to inhibitors, are needed.

The modified FIX polypeptides provided herein exhibit improved properties, including improved pharmacokinetic properties, such as increased serum half-life; increased resistance to inhibitors, such as antithrombin III (AT-III), heparin and the AT-III/heparin complex; increased catalytic activity; or any combination thereof. Hence, provided are modified FIX polypeptides that have increased coagulant activity. Accordingly, these polypeptides have a variety of uses and applications, for example, as therapeutics for modulating hemostasis. The following discussion provides a review of the coagulation process and the role of Factor IX in this process, before a discussion of factor IX, and modifications thereof.

Hemostasis is the physiological mechanism that stems the bleeding that results from injury to the vasculature. Normal hemostasis depends on cellular components and soluble plasma proteins, and involves a series of signaling events that ultimately leads to the formation of a blood clot. Coagulation is quickly initiated after an injury occurs to the blood vessel and endothelial cells are damaged. In the primary phase of coagulation, platelets are activated to form a hemostatic plug at the site of injury. Secondary hemostasis follows involving plasma coagulation factors, which act in a proteolytic cascade resulting in the formation of fibrin strands which strengthen the platelet plug.

Upon vessel injury, the blood flow to the immediate injured area is restricted by vascular constriction allowing platelets to adhere to the newly-exposed fibrillar collagen on the subendothelial connective tissue. This adhesion is dependent upon the von Willebrand factor (vWF), which binds to the endothelium within three seconds of injury, thereby facilitating platelet adhesion and aggregation. Activation of the aggregated platelets results in the secretion of a variety of factors, including ADP, ATP, thromboxane and serotonin. Adhesion molecules, fibrinogen, vWF, thrombospondin and fibronectin also are released. Such secretion promotes additional adhesion and aggregation of platelets, increased platelet activation and blood vessel constriction, and exposure of anionic phospholipids on the platelet surface that serve as platforms for the assembly of blood coagulation enzyme complexes. The platelets change shape leading to pseudopodia formation, which further facilitates aggregation to other platelets resulting in a loose platelet plug.

A clotting cascade of peptidases (the coagulation cascade) is simultaneously initiated. The coagulation cascade involves a series of activation events involving proteolytic cleavage. In such a cascade, an inactive protein of a serine protease (also called a zymogen) is converted to an active protease by cleavage of one or more peptide bonds, which then serves as the activating protease for the next zymogen molecule in the cascade, ultimately resulting in clot formation by the cross-linking of fibrin. For example, the cascade generates activated molecules such as thrombin (from cleavage of prothrombin), which further activates platelets, and also generates fibrin from cleavage of fibrinogen. Fibrin then forms a cross-linked polymer around the platelet plug to stabilize the clot. Upon repair of the injury, fibrin is digested by the fibrinolytic system, the major components of which are plasminogen and tissue-type plasminogen activator (tPA). Both of these proteins are incorporated into polymerizing fibrin, where they interact to generate plasmin, which, in turn, acts on fibrin to dissolve the preformed clot. During clot formation, coagulation factor inhibitors also circulate through the blood to prevent clot formation beyond the injury site.

The interaction of the system, from injury to clot formation and subsequent fibrinolysis, is described below.

1. Platelet Adhesion and Aggregation

The clotting of blood is actively circumvented under normal conditions. The vascular endothelium supports vasodilation, inhibits platelet adhesion and activation, suppresses coagulation, enhances fibrin cleavage and is anti-inflammatory in character. Vascular endothelial cells secrete molecules such as nitrous oxide (NO) and prostacyclin, which inhibit platelet aggregation and dilate blood vessels. Release of these molecules activates soluble guanylate cyclases (sGC) and cGMP-dependent protein kinase I (cGKI) and increases cyclic guanosine monophosphate (cGMP) levels, which cause relaxation of the smooth muscle in the vessel wall. Furthermore, endothelial cells express cell-surface ADPases, such as CD39, which control platelet activation and aggregation by converting ADP released from platelets into adenine nucleotide platelet inhibitors. The endothelium also plays an important role in the regulation of the enzymes in the fibrinolytic cascade. Endothelial cells directly promote the generation of plasmin through the expression of receptors of plasminogen (annexin II) and urokinase, as well as the secretion of tissue-type and urokinase plasminogen activators, all of which promote clot clearance. In a final layer of prothrombotic regulation, endothelial cells play an active role in inhibiting the coagulation cascade by producing heparan sulfate, which increases the kinetics of antithrombin III inhibition of thrombin and other coagulation factors.

Under acute vascular trauma, however, vasoconstrictor mechanisms predominate and the endothelium becomes prothrombotic, procoagulatory and proinflammatory in nature. This is achieved by a reduction of endothelial dilating agents: adenosine, NO and prostacyclin; and the direct action of ADP, serotonin and thromboxane on vascular smooth muscle cells to elicit their contraction (Becker et al., (2000) Z. Kardiol. 89:160-167). The chief trigger for the change in endothelial function that leads to the formation of hemostatic thrombus is the loss of the endothelial cell barrier between blood and extracellular matrix (ECM) components (Ruggeri (2002) Nat. Med. 8:1227-1234). Circulating platelets identify and discriminate areas of endothelial lesions and adhere to the exposed sub endothelium. Their interaction with the various thrombogenic substrates and locally-generated or released agonists results in platelet activation. This process is described as possessing two stages, 1) adhesion: the initial tethering to a surface, and 2) aggregation: the platelet-platelet cohesion (Savage et al. (2001) Curr. Opin. Hematol. 8:270-276).

Platelet adhesion is initiated when the circulating platelets bind to exposed collagen through interaction with collagen binding proteins on the cell surface, and through interaction with vWF, also present on the endothelium. vWF protein is a multimeric structure of variable size, secreted in two directions by the endothelium; basolaterally and into the bloodstream. vWF also binds to factor VIII, which is important in the stabilization of factor VIII and its survival in the circulation.

Platelet adhesion and subsequent activation is achieved when vWF binds via its A1 domain to GPIb (part of the platelet glycoprotein receptor complex GPIb-IX-V). The interaction between vWF and GPIb is regulated by shear force such that an increase in the shear stress results in a corresponding increase in the affinity of vWF for GPIb. Integrin α1β2, also known on leukocytes as VLA-2, is the major collagen receptor on platelets, and engagement through this receptor generates the intracellular signals that contribute to platelet activation. Binding through α1β2 facilitates the engagement of the lower-affinity collagen receptor, GP VI. This is part of the immunoglobulin superfamily and is the receptor that generates the most potent intracellular signals for platelet activation. Platelet activation results in the release of adenosine diphosphate (ADP), which is converted to thromboxane A2.

Platelet activation also results in the surface expression of platelet glycoprotein IIb-IIIa (GP IIb-IIIa) receptors, also known as platelet integrin αIIbβ3. GP IIb-IIIa receptors allow the adherence of platelets to each other (i.e., aggregation) by virtue of fibrinogen molecules linking the platelets through these receptors. This results in the formation of a platelet plug at the site of injury to help prevent further blood loss, while the damaged vascular tissue releases factors that initiate the coagulation cascade and the formation of a stabilizing fibrin mesh around the platelet plug.

2. Coagulation Cascade

The coagulation pathway is a proteolytic pathway where each enzyme is present in the plasma as a zymogen, or inactive form. Cleavage of the zymogen is regulated to release the active form from the precursor molecule. The pathway functions as a series of positive and negative feedback loops that control the activation process, where the ultimate goal is to produce thrombin, which can then convert soluble fibrinogen into fibrin to form a clot. The coagulation factors, and other proteins, participate in blood coagulation through one or more of the intrinsic, extrinsic or common pathway of coagulation. As discussed below, these pathways are interconnected, and blood coagulation likely occurs through a cell-based model of activation.

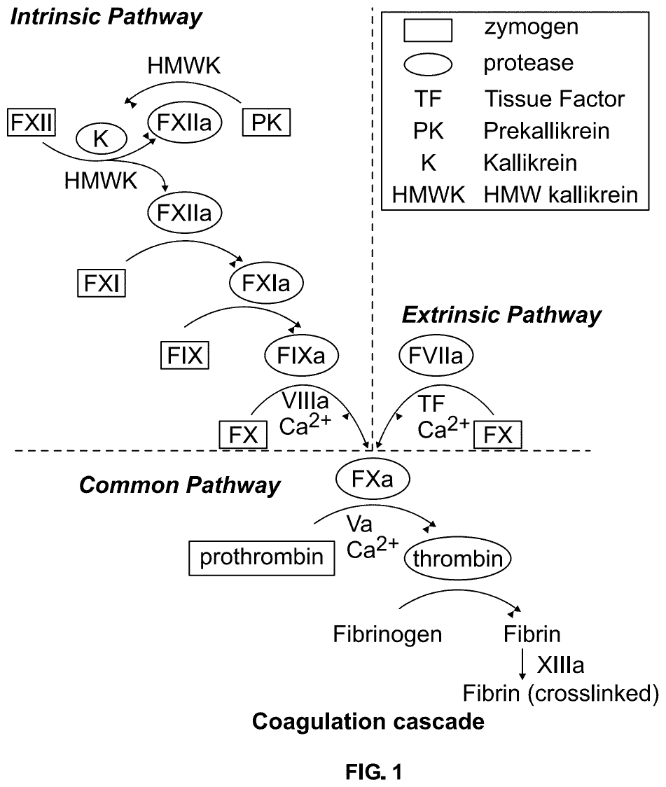

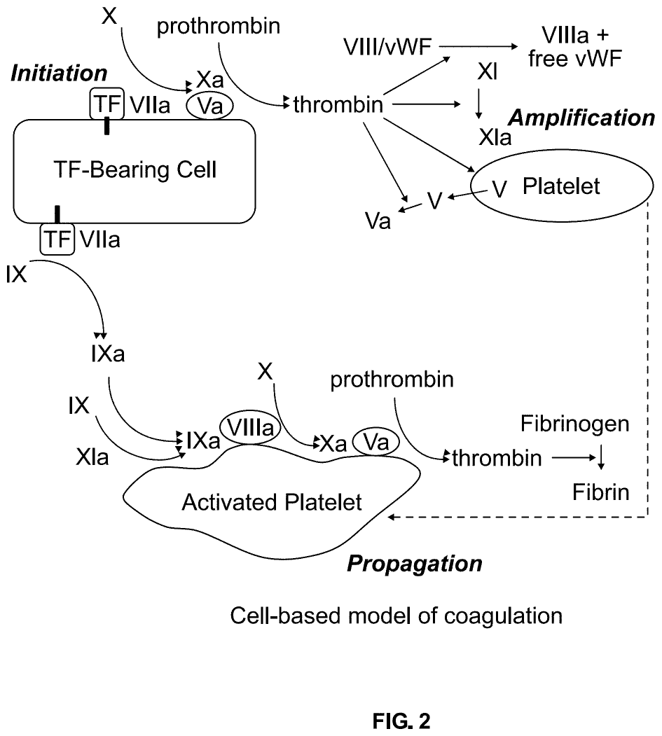

The generation of thrombin has historically been divided into three pathways, the intrinsic (indicating that all components of the pathway are intrinsic to plasma) and extrinsic (indicating that one or more components of the pathway are extrinsic to plasma) pathways that provide alternative routes for the generation of activated Factor X (FXa), and the final common pathway which results in thrombin formation (FIG. 1). These pathways participate together in an interconnected and interdependent process to effect coagulation. A cell-based model of coagulation was developed that describes these pathways (FIG. 2) (Hoffman et al. (2001) J. Thromb. Haemost. 85:958-965). In this model, the “extrinsic” and “intrinsic” pathways are effected on different cell surfaces; the tissue factor (TF)-bearing cell and the platelet, respectively. The process of coagulation is separated into distinct phases, initiation, amplification and propagation, during which the extrinsic and intrinsic pathways function at various stages to produce the large burst of thrombin required to convert sufficient quantities of fibrinogen to fibrin for clot formation.

a. Initiation

FVII is considered to be the coagulation factor responsible for initiating the coagulation cascade, which initiation is dependent on its interaction with TF. TF is a transmembrane glycoprotein expressed by a variety of cells such as smooth muscle cells, fibroblasts, monocytes, lymphocytes, granulocytes, platelets and endothelial cells. Myeloid cells and endothelial cells only express TF when they are stimulated, such as by proinflammatory cytokines. Smooth muscle cells and fibroblasts, however, express TF constitutively. Accordingly, once these cells come in contact with the bloodstream following tissue injury, the coagulation cascade is rapidly initiated by the binding of TF with Factor VII or FVIIa in the plasma. TF/FVIIa complexes can be formed by the direct binding of FVIIa to TF, or by the binding of FVII to TF and then the subsequent activation of FVII to FVIIa by a plasma protease, such as FXa, FIXa, FXIIa, or FVIIa itself. The TF/FVIIa complex remains anchored to the TF-bearing cell where it activates small amounts FX into FXa in what is known as the “extrinsic pathway” of coagulation.

The TF/FVIIa complex also cleaves small amounts of FIX into FIXa. FXa associates with its cofactor FVa to also form a complex on the TF-bearing cell that can then covert prothrombin to thrombin. The small amount of thrombin produced is, however, inadequate to support the required fibrin formation for complete clotting. Additionally, any active FXa and FIXa are inhibited in the circulation by antithrombin III (AT-III) and other serpins, which are discussed in more detail below. This would normally prevent clot formation in the circulation. In the presence of injury, however, damage to the vasculature results in platelet aggregation and activation at this site of thrombin formation, thereby allowing for amplification of the coagulation signal.

b. Amplification

Amplification takes place when thrombin binds to and activates the platelets. The activated platelets release FV from their alpha granules, which is activated by thrombin to FVa. Thrombin also releases and activates FVIII from the FVIII/vWF complex on the platelet membrane, and cleaves FXI into FXIa. These reactions generate activated platelets that have FVa, FVIIIa and FIXa on their surface, which set the stage for a large burst of thrombin generation during the propagation stage.

c. Propagation

Propagation of coagulation occurs on the surface of large numbers of platelets at the site of injury. As described above, the activated platelets have FXIa, FVIIIa and FVa on their surface. It is here that the extrinsic pathway is effected. FXIa activates FIX to FIXa, which can then bind with FVIIIa. This process, in addition to the small amounts of FIXa that is generated by cleavage of FIX by the TF/FVIIa complex on the TF-bearing cell, generates a large amount FIXa in complex with its cofactor, FVIIIa, calcium and a suitable phospholipid surface. This complex is termed the tenase or Xase complex, and it cleaves and activates the Factor X (FX) to Factor Xa (FXa). The FXa molecules bind to FVa to generate the prothrombinase complexes that activate prothrombin to thrombin. Thrombin acts in a positive feedback loop to activate even more platelets and again initiates the processes described for the amplification phase.

Very shortly, there are sufficient numbers of activated platelets with the appropriate complexes to generate the burst of thrombin that is large enough to generate sufficient amounts of fibrin from fibrinogen to form a hemostatic fibrin clot. Fibrinogen is a dimer soluble in plasma which, when cleaved by thrombin, releases fibrinopeptide A and fibrinopeptide B. Fibrinopeptide B is then cleaved by thrombin, and the fibrin monomers formed by this second proteolytic cleavage spontaneously forms an insoluble gel. The polymerized fibrin is held together by noncovalent and electrostatic forces and is stabilized by the transamidating enzyme factor XIIIa (FXIIIa), produced by the cleavage of FXIII by thrombin. Thrombin also activates TAFI, which inhibits fibrinolysis by reducing plasmin generation at the clot surface. Additionally, thrombin itself is incorporated into the structure of the clot for further stabilization. These insoluble fibrin aggregates (clots), together with aggregated platelets (thrombi), block the damaged blood vessel and prevent further bleeding.

3. Regulation of Coagulation

During coagulation, the cascade is regulated by constitutive and stimulated processes to inhibit further clot formation. Regulation is important to a) limit ischemia of tissues by fibrin clot formation, and b) prevent widespread thrombosis by localizing the clot formation only to the site of tissue injury.

Regulation is achieved by the actions of several inhibitory molecules. For example, antithrombin III (AT-III) and tissue factor pathway inhibitor (TFPI) work constitutively to inhibit factors in the coagulation cascade. TFPI predominantly inhibits FXa and FVIIa/TF complex. In contrast, AT-III, which is a serine protease inhibitor (serpin), predominantly inhibits thrombin, FXa, and FIXa. The inhibition of these coagulation factors by AT-III is enhanced greatly by heparin, which binds AT-III to induce an activating conformational change that accelerates the inhibitory reaction. Heparin also can inhibit the activity of the FIXa/FVIIIa complex in an AT-III-independent manner (Yuan et al., (2005) Biochemistry 44:3615-3625). An additional factor, Protein C, which is stimulated via platelet activation, regulates coagulation by proteolytic cleavage and inactivation of FVa and FVIIIa. Protein S enhances the activity of Protein C. Further, another factor which contributes to coagulation inhibition is the integral membrane protein thrombomodulin, which is produced by vascular endothelial cells and serves as a receptor for thrombin. Binding of thrombin to thrombomodulin inhibits thrombin procoagulant activities and also contributes to protein C activation.

Fibrinolysis, the breakdown of the fibrin clot, also provides a mechanism for regulating coagulation. The cross-linked fibrin multimers in a clot are broken down to soluble polypeptides by plasmin, a serine protease. Plasmin can be generated from its inactive precursor plasminogen and recruited to the site of a fibrin clot in two ways: by interaction with tissue plasminogen activator (tPA) at the surface of a fibrin clot, and by interaction with urokinase plasminogen activator (uPA) at a cell surface. The first mechanism appears to be the major one responsible for the dissolution of clots within blood vessels. The second, although capable of mediating clot dissolution, can play a major role in tissue remodeling, cell migration, and inflammation.

Clot dissolution also is regulated in two ways. First, efficient plasmin activation and fibrinolysis occur only in complexes formed at the clot surface or on a cell membrane, while proteins free in the blood are inefficient catalysts and are rapidly inactivated. Second, plasminogen activators and plasmin are inactivated by molecules such as plasminogen activator inhibitor type 1 (PAI-1) and PAI-2 which act on the plasminogen activators, and α2-antiplasmin and α 2-macroglobulin that inactivate plasmin. Under normal circumstances, the timely balance between coagulation and fibrinolysis results in the efficient formation and clearing of clots following vascular injury, while simultaneously preventing unwanted thrombotic or bleeding episodes.

### C. FACTOR IX (FIX) STRUCTURE AND FUNCTION

Modified FIX polypeptides described herein with improved activities or functions are for use in the prophylactic subcutaneous methods and regimens. FIX is a polypeptide that is involved in the coagulation cascade. The role of FIX in the coagulation cascade is related to its structure and mechanism of activation. It is understood that the modulation of coagulation by modified FIX polypeptides provided herein also is linked to its structure and mechanism of activation. These features can be the same as an unmodified FIX polypeptide. In other cases, these features can be modified in a FIX polypeptide provided herein, thus resulting in a polypeptide with altered or improved activities or properties. For example, modification of a FIX polypeptide can alter one or more activities of a FIX polypeptide. For example, provided are modified FIX polypeptides that exhibit increased levels of glycosylation compared to a wild-type FIX polypeptide. The modified FIX polypeptides can thus exhibit improved pharmacokinetic properties, such as reduced clearance and increased serum half-life compared to a wild-type FIX polypeptide, when tested using in vivo assays. Also provided are modified FIX polypeptides that exhibit increased resistance to inhibitors, such as AT-III, heparin and the AT-III/heparin complex; and/or increased catalytic activity. Thus, provided are modified FIX polypeptides that exhibit improved therapeutic properties compared to an unmodified FIX polypeptide. A summary of structural and functional features of FIX polypeptides and modified FIX polypeptides are described below.

Factor IX is a vitamin K-dependent serine protease and is an important coagulation factor in hemostasis. It is synthesized as a single chain zymogen in the liver and circulates in the blood in this inactivated state until activated as part of the coagulation cascade. Following activation from the FIX zymogen to activated FIX (FIXa) by FXIa or the TF/FVIIa complex, FIXa binds its cofactor, FVIIIa. The resulting FIXa/FVIIIa complex binds and activates FX to FXa, thus continuing the coagulation cascade described above to establish hemostasis. The concentration of FIX in the blood is approximately 4-5 μg/mL, and it has a half-life of approximately 18-24 hours.

Hemophilia B, also known as Christmas disease or Factor IX deficiency, is caused by a deficiency or dysfunction of FIX resulting from any one or more of a variety of mutations in the FIX gene. While less prevalent than Hemophilia A, Hemophilia B remains a significant disease in which recurrent joint bleeds can lead to synovial hypertrophy, chronic synovitis, with destruction of synovium, cartilage, and bone leading to chronic pain, stiffness of the joints, and limitation of movement because of progressive severe joint damage. Recurrent muscle bleeds also produce acute pain, swelling, and limitation of movement, while bleeding at other sites can contribute to morbidity and mortality. Treatment is typically by replacement therapy with recombinant FIX (rFIX). Provided herein are modified FIX polypeptides that are designed to have increased coagulation activity upon activation, and that can serve as improved therapeutics to treat diseases and conditions amenable to factor IX therapy, such as Hemophilia B.

1. FIX Structure

The human FIX gene is located on the X chromosome and is approximately 34 kb long with eight exons. The human FIX transcript is 2803 nucleotides and contains a short 5′ untranslated region, an open reading frame (including stop codon) of 1383 nucleotides and a 3′ untranslated region. The 1383 nucleotide open reading frame (or FIX mRNA; SEQ ID NO:1) encodes a 461 amino acid precursor polypeptide (Swiss-Prot accession no. P00740; SEQ ID NO:2) containing a 28 amino acid N-terminal signal peptide (amino acids 1-28 of SEQ ID NO:2) that directs the factor IX polypeptide to the cellular secretory pathway. In addition the hydrophobic signal peptide, the FIX precursor polypeptide also contains an 18 amino acid propeptide (amino acid residues 29-46 of SEQ ID NO:2) that, when cleaved, releases the 415 amino acid mature polypeptide (SEQ ID NO:3) that circulates in the blood as a zymogen until activation to FIXa. In addition to the signal peptide and propeptide, the FIX precursor also contains the following segments and domains: a Gla domain (amino acids 47-92 of SEQ ID NO:2, corresponding to amino acids 1-46 of the mature FIX protein set forth in SEQ ID NO:3), epidermal growth factor (EGF)-like domain 1 (EGF1; amino acids 93-129 of SEQ ID NO:2, corresponding to amino acids 47-83 of the mature FIX protein set forth in SEQ ID NO:3), EGF2 (amino acids 130-171 of SEQ ID NO:2, corresponding to amino acids 84-125 of the mature FIX protein set forth in SEQ ID NO:3), a light chain (amino acids 47-191 of SEQ ID NO:2, corresponding to amino acids 1-145 of the mature FIX protein set forth in SEQ ID NO:3), an activation peptide (amino acids 192-226 of SEQ ID NO:2, corresponding to amino acids 146-180 of the mature FIX protein set forth in SEQ ID NO:3), a heavy chain (amino acids 227-461 of SEQ ID NO:2, corresponding to amino acids 181-415 of the mature FIX protein set forth in SEQ ID NO:3) and a serine protease domain (amino acids 227-459 of SEQ ID NO:2, corresponding to amino acids 181-413 of the mature FIX protein set forth in SEQ ID NO:3).

Like other vitamin K-dependent proteins, such as prothrombin, coagulation factors VII and X, and proteins C, S, and Z, the Gla domain of FIX is a membrane binding motif which, in the presence of calcium ions, interacts with the phospholipid membranes of cells. The vitamin K-dependent proteins require vitamin K for the posttranslational synthesis of γ-carboxyglutamic acid, an amino acid clustered in the Gla domain of these proteins. The FIX Gla domain has 12 glutamic residues, each of which are potential carboxylation sites. Many of them are, therefore, modified by carboxylation to generate γ-carboxyglutamic acid residues. There are a total of eight Ca²⁺ binding sites, of both high and low affinity, in the FIX Gla domain that, when occupied by calcium ions, facilitate correct folding of the Gla domain to expose hydrophobic residues in the FIX polypeptide that are inserted into the lipid bilayer to effect binding to the membrane.

In addition to the Gla domain, the FIX polypeptide also contains two EGF-like domains. Each EGF-like domain contains six highly conserved cysteine residues that form three disulfide bonds in each domain in the same pattern observed in the EGF protein. The first EGF-like domain (EGF1) is a calcium-binding EGF domain containing a high affinity Ca′ binding site (Rao et al., (1995) Cell 82:131-141) that, when occupied by a calcium ion, contributes to the correct folding of the molecule and promotes biological activity. The second EGF domain (EGF2) does not contain a calcium binding site.

The serine protease domain, or catalytic domain, of FIX is the domain responsible for the proteolytic activity of FIXa. Like other serine proteases, FIX contains a serine protease catalytic triad composed of H221, D269 and S365 (corresponding to H57, D102 and S195 by chymotrypsin numbering).

Activation of mature FIX to FIXa is effected by proteolytic cleavage of the R145-A146 bonds and R180-V181 bonds (numbering relative to the mature FIX polypeptide set forth in SEQ ID NO:3), releasing the activation peptide that corresponds to amino acids 146-180 of the mature FIX protein set forth in SEQ ID NO:3. Thus, following activation, FIXa consists of two chains; the light chain and heavy chain. The light chain contains the Gla domain, EGF1 and EGF2 domains, and the heavy chain contains the protease domain. The two chains are held together by a single disulfide bond between C132 and C289.

2. FIX Post-Translational Modification

The Factor IX precursor polypeptide undergoes extensive post-translational modification to become the mature zymogen that is secreted into the blood. Such post-translational modifications include γ-carboxylation, β-hydroxylation, cleavage of the signal peptide and propeptide, O- and N-linked glycosylation, sulfation and phosphorylation. The N-terminal signal peptide directs the polypeptide to the endoplasmic reticulum (ER), after which it is cleaved. Immediately prior to secretion from the cell, the propeptide is cleaved by processing proteases, such as, for example, PACE/furin, that recognize at least two arginine residues within four amino acids prior to the cleavage site.

A single enzyme, vitamin K-dependent gamma-carboxylase, catalyzes the γ-carboxylation FIX in the ER (Berkner (2000) J. Nutr. 130:1877-1880). In the carboxylation reaction, the γ-carboxylase binds to the FIX propeptide and catalyzes a second carboxylation on the γ-carbon of the glutamic acid residues (i.e., Glu to γ-carboxyglutamyl or Gla) in the Gla domain of the polypeptide. Assuming all glutamic acid residues are γ-carboxylated, FIX contains 12 Gla residues, where the first 10 are at homologous positions of other vitamin K-dependent proteins. The Gla domain of FIX then processively carboxylates all glutamates in the cluster before releasing the substrate (Morris et al. (1995) J. Biol. Chem. 270(51):30491-30498; Berkner (2000) J. Nutr. 130:1877-1880; Stenina et al. (2001) Biochemistry 40:10301-10309).

FIX also is partially β-hydroxylated. This modification is performed by a dioxygenase, which hydroxylates the β-carbon of D64 (corresponding to the mature FIX polypeptide set forth in SEQ ID NO:3) in EGF1. Approximately one third of human FIX polypeptides are β-hydroxylated. Although D64 contributes to the high affinity Ca²⁺ binding site in the EGF1 domain of FIX, the hydroxylation of this residue does not appear to be necessary for Ca²⁺ binding, nor for biological activity (Derian et al., (1989) J. Biol. Chem. 264:6615-6618; Sunnerhagen et al., (1993) J. Biol. Chem. 268: 23339-23344). Additional post-translational modifications include sulfonation at the tyrosine at position 155, and phosphorylation at the serine residue at position 158. These post-translational modifications of Factor IX have been implicated in contributing to in vivo recovery of FIX (Kaufman (1998) J. Thromb. Haemost. 79:1068-1079; U.S. Pat. No. 7,575,897).

FIX is N-linked glycosylated at asparagine residues in the activation peptide corresponding to N157 and N167 of the mature FIX polypeptide set forth in SEQ ID NO:3. Post-translational modification also results in the serine residue at position 53 (corresponding to the mature FIX polypeptide set forth in SEQ ID NO:3) having O-linked disaccharides and trisaccharides, while the serine residue at position 61 contains an O-linked tertrasaccharide. (Nishimura et al., (1989) J. Biol. Chem. 264:20320-20325; Harris et al., (1993) Biochemistry 32:6539-6547). Additionally, the threonine residues at amino acid positions 159 and 169 (corresponding to the mature FIX polypeptide set forth in SEQ ID NO:3) are O-glycosylated (Agarwala et al., (1994) Biochemistry 33:5167-5171). The threonine residues at amino acid positions 172 and 179 also may be O-glycosylated.

3. FIX Activation

Factor IX circulates predominantly as a zymogen with minimal proteolytic activity until it is activated by proteolytic cleavage. Activation can be effected by the TF/FVIIa complex or Factor XIa. Activation by TF/FVIIa is through the intrinsic pathway, while activation by FXIa is through the extrinsic pathway, described above. The process of activation appears to be sequential with initial cleavage of the Arg145-Ala146 bond, followed by cleavage of the Arg180-Val181 bond (Schmidt et al. (2003) Trends Cardio. Med. 13:39-45). The proteolytic cleavage releases the activation peptide, forming the two-chain FIXa molecule containing the light chain (corresponding to amino acid positions 1-145 of SEQ ID NO:3) and heavy chain (corresponding to amino acid positions 181-415 of SEQ ID NO:3) held together by a disulfide bond between the two cysteine residues at amino acid positions 132 and 289 (numbering corresponding to the mature FIX polypeptide set forth in SEQ ID NO:3).

At least two exosites in FX appear to be involved in binding to TF in the TF/FVIIa complex to form the FIX/TF/FVIIa ternary complex (Chen et al., (2002) J. Thromb. Haemost. 88:74-82). Studies indicate that the EGF1 domain of FIX is required for FIX activation by the TF/FVIIa complex. For example, mutation of G48 (relative to the mature FIX polypeptide set forth in SEQ ID NO:3) in the EGF1 domain of FIX reduces its activation by TF/FVIIa (Wu et al., (2000) J. Thromb. Haemost. 84:626-634). Further, the EGF1 domain of FIX has been shown to interact with TF in the TF/FVIIa complex (Zhong et al., (2002) J. Biol. Chem. 277:3622). In contrast, however, the EGF1 domain does not appear to be required for FIX activation by FXIa. The Gla domain also is involved in binding to the TF/FVIIa complex and, therefore, in activation. The Gla domain of FIX interacts with the same region in TF as FX, which also is activated by the TF/FVIIa complex (Kirchhofer et al., (2000) Biochem. 39:7380-7387).

Following cleavage and release of the activation peptide, a new amino terminus at V181 (corresponding to the mature FIX polypeptide set forth in SEQ ID NO:3; V16 by chymotrypsin numbering) is generated. Release of the activation peptide facilitates a conformational change whereby the amino group of V181 inserts into the active site and forms a salt bridge with the side chain carboxylate of D364. Such a change is required for conversion of the zymogen state to an active state, as the change converts the hydroxyl side chain of 5365 to a reactive species that is able to hydrolyze the cleavage site of its substrate, FX. The activated FIXa polypeptide remains in a zymogen-like conformation until additional conformational changes are induced, such as by binding with FVIIIa, to generate a FIXa polypeptide with maximal catalytic activity.

4. FIX Function

FIX plays an important role in the coagulation pathway and a deficiency or absence of FIX activity leads to hemophilia B. Once activated from FIX to FIXa, FIXa in turn functions to activate the large amounts of FX to FXa that are required for coagulation. To do so, FIXa must first bind to its cofactor, Factor VIIIa, to form the FIXa/FVIIIa complex, also called the intrinsic tenase complex, on the phospholipid surface of the activated platelet. Both the Gla domain and EGF2 domain of FIX are important for stable binding to phospholipids. The FIXa/FVIIIa complex then binds FX to cleave this coagulation factor to form FIXa.

FIXa is virtually inactive in the absence of its cofactor, FVIIIa, and physiologic substrate, FX. Experimental studies indicate that this can be attributed mainly to the 99-loop. When FIXa is not bound by its cofactor, Y177 locks the 99-loop in an inactive conformation in which the side chains of Y99 and K98 (by chymotrypsin numbering, corresponding to Y266 and K265 of the mature FIX polypeptide set forth in SEQ ID NO:3) impede substrate binding. Binding of FVIIIa to FIXa unlocks and releases this zymogen-like conformation, and FX is then able to associate with the FIXa/FVIIIa complex and rearrange the unlocked 99-loop, subsequently binding to the active site cleft (Sichler et al., (2003) J. Biol. Chem. 278:4121-4126). The binding of FIXa to phospholipids and the presence of Ca²⁺ further enhances the reaction.

Several models of the FIXa/FVIIIa interaction have been proposed (see, e.g., Autin et al., (2005) J. Thromb. Haemost. 3:2044-2056; Stoilova-McPhie et al., (2002) Blood 99: 1215-1223; Bajaj et al., (2001) J. Biol. Chem. 276:16302-16309; Schmidt et al., (2003) Trends Cardiovasc. Med. 13:39-45). FIXa binds to FVIIIa in an interaction involving more than one domain of the FIXa polypeptide. FVIIIa is a heterodimer composed of three non-covalently associated chains: A1, A2 and A3-C1-C2. A3-C1-C2 also is referred to as the light chain. The protease domain of FIXa appears to interact with the A2 subunit of FVIIIa. Studies indicate that the 293-helix (126-helix by chymotrypsin numbering), 330-helix (162-helix by chymotrypsin numbering) and N346 (N178) by chymotrypsin numbering) of FIXa are involved in the interaction with the A2 subunit of FVIIIa. The EGF1/EGF2 domains of FIXa interact with the A3 subunit of FVIIIa. Further, it is postulated that the Gla domain of FIXa interacts with the C2 domain of FVIIIa. Calcium ions and phospholipids also contribute to binding of FIXa and FVIIIa. For example, the presence of phospholipids increases the binding of FIXa to FVIIIa by approximately 2000-fold (Mathur et al., (1997) J. Biol. Chem. 272(37):23418-23426). Following binding of FX by the FIXa/FVIIIa complex, the protease domain (or catalytic domain) of FIXa is responsible for cleavage of FX at R194-I195 to form FXa.

The activity of FIXa is regulated by inhibitory molecules, such as the AT-III/heparin complex, as discussed above, and other clearance mechanisms, such as the low-density lipoprotein receptor-related protein (LRP). LRP is a membrane glycoprotein that is expressed on a variety of tissues, including liver, brain, placenta and lung. LRP binds a wide range of proteins and complexes in addition to FIXa, including, but not limited to, apolipoproteins, lipases, proteinases, proteinase-inhibitor complexes, and matrix proteins. The zymogen or inactive form of FIX does not bind LRP. Rather, upon activation, an LRP-binding site is exposed (Neels et al., (2000) Blood 96:3459-3465). This binding site is located in a loop in the protease domain spanning residues 342 to 346 of the mature FIX polypeptide set forth in SEQ ID NO:3 (Rohlena et al., (2003) J. Biol. Chem. 278:9394-9401).

5. FIX as a Biopharmaceutical

Factor IX is integrally involved in the blood coagulation process, where, in its activated form (FIXa), it forms a tenase complex with FVIIIa and activates FX to FXa. FXa, in conjunction with phospholipids, calcium and FVa, converts prothrombin to thrombin, which in turn cleaves fibrinogen to fibrin monomers, thus facilitating the formation of a rigid mesh clot. Many studies have demonstrated the ability of exogenous FIX to promote blood clotting in patients with hemophilia. For example, hemophilia B patients, who are deficient in FIX, can be treated by replacement therapy with exogenous FIX. Early replacement therapies utilized plasma purified FIX, such as therapeutics marketed as MonoNine® Factor IX and Alpha-nine-SD® Factor IX. Plasma purified FIX complex therapeutics also have been used, including Bebulin® VH, a purified concentrate of FIX with FX and low amounts of FVII; Konyne® 80 (Bayer), a purified concentrate of FIX, with FII, FX, and low levels of FVII; PROPLEX® T (Baxter International), a heat treated product prepared from pooled normal human plasma containing FIX with FII, FVII, and FX; and Profilnine SD® (Alpha Therapeutic Corporation). A human recombinant Factor IX (BeneFIX® Coagulation Factor IX (Recombinant), Pfizer) is approved for use in the control and prevention of bleeding episodes in hemophilia B patients, including control and prevention of bleeding in surgical settings. BeneFIX® Coagulation Factor IX (Recombinant) has an amino acid sequence set forth in SEQ ID NO:20, and is identical to the Ala148 allelic form of plasma-derived Factor IX. Thus, compared to the wild-type FIX polypeptide set forth in SEQ ID NO:3, BeneFIX® Coagulation Factor IX (Recombinant) contains a T148A mutation.

In addition to its use as a procoagulant, inactive forms of FIX, or forms with reduced catalytic activity, can be used as an anticoagulant, such as in the treatment of thrombotic diseases and conditions.

Typically, FIX is administered intravenously, but also can be administered orally, systemically, buccally, transdermally, intramuscularly and subcutaneously. FIX can be administered once or multiple times. Generally, multiple administrations are used in treatment regimens with FIX to effect coagulation.

As discussed herein below, modified FIX polypeptides provided herein also can be used in any treatment or pharmaceutical method in which an unmodified or wild-type or other therapeutically active FIX polypeptide is known to be used. In such uses, methods and processes, the modified FIX polypeptides provided herein exhibit improved properties compared to a wild-type or the unmodified FIX polypeptide.

### D. AAV GENE THERAPY VECTORS AND CONSTRUCTS FOR GENE THERAPY

Provided are compositions containing nucleic acid molecules encoding the modified FIX polypeptides and vectors encoding them that are suitable for gene therapy. Rather than deliver the protein, nucleic acid encoding the protein is administered in vivo, such as systemically or by other route, or ex vivo, such as by removal of cells, including lymphocytes, introduction of the nucleic therein, and reintroduction into the host or a compatible recipient. Modified FIX polypeptides can be administered as nucleic acid molecules encoding modified FIX polypeptides, including ex vivo techniques and direct in vivo expression. Nucleic acids can be delivered to cells and tissues by any method known to those of skill in the art including systemic administration, and also direct injection into the liver parenchyma following compartmentalization (see, U.S. Pat. No. 9,821,114),

The methods for administering modified FIX polypeptides by expression of encoding nucleic acid molecules include administration of recombinant vectors. Vectors are designed to remain episomal, such as by inclusion of an origin of replication or are designed to integrate into a chromosome in the cell. Modified FIX polypeptides also can be used in ex vivo gene expression therapy using non-viral vectors. For example, cells can be engineered to express a modified FIX polypeptide, such as by integrating a modified FIX polypeptide encoding-nucleic acid into a genomic location, either operatively linked to regulatory sequences or such that it is placed operatively linked to regulatory sequences in a genomic location. Such cells then can be administered locally or systemically to a subject, such as a patient in need of treatment.

As provided herein, the nucleic acid encoding the modified FIX polypeptides are provided in an AAV vector. The AAV vectors have been generated and selected to have increased tropism for liver cells, whereby upon administration the vector is taken up by hepatocytes and the encoded FIX polypeptide is expressed and secreted into systemic circulation. The nucleic acid encoding the modified FIX polypeptide includes an intron, generally all or a portion of the first intron; this results in increased expression. The nucleic acid also encodes a signal sequence and expression can be under control of liver specific regulatory sequences. The AAV vector employed is designed or generated to have improved properties compared to naturally-occurring serotypes.

Adeno-associated virus (AAV), a member of the Parvovirus family, is a small non-enveloped, icosahedral virus with single-stranded linear DNA genomes of 4.7 kilobases (kb). In its native state, AAV is replication-defective. It requires a helper virus, typically adenovirus, to provide necessary protein factors for replication. AAV is a small, non-enveloped, non-pathogenic, helper virus dependent single-stranded DNA virus; there are numerous serotypes having varying tissue tropisms and transduction efficiencies. AAV2 and AAV8 have been used to target the liver to achieve long-term expression of encoded therapeutic proteins. In its native state, the AAV life cycle includes a latent phase during which AAV genomes, after infection, are site-specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains. Rep and Cap can be replaced with heterologous nucleic acid encoding a product(s) of interest.

To produce AAV vectors with altered tropism, AAV vectors encoding variant capsid were generated and screened. DNA encoding capsids from 8 AAV serotypes were shuffled to produce chimeric capsids, which were screened from increased transduction efficiency. The method for production and selection included, for example, the steps of a) generating a library of variant AAV capsid polypeptide genes in which the variant AAV capsid polypeptide genes include a plurality of variant AAV capsid polypeptide genes comprising sequences from more than one non-variant parent capsid polypeptide; b) generating an AAV vector library of replication competent AAV vectors by cloning the variant AAV capsid polypeptide gene library into the AAV vectors; c) screening the library to identify capsid polypeptides that have increased transduction or tropism for a particular tissue or organ; and d) selecting the vectors, and hence the capsids, that have the desired tropism. For the capsids and AAV vectors used herein for encoding the modified FIX, a library was prepared and screened for increased tropism for pancreatic islet cells compared to a non-variant parent capsid polypeptide. Among the candidates tested in our study, 3 chimeric variants exhibit considerably improved transduction capacity of human islet cells—particularly of β cells. In addition, these variants exhibit improved transduction in other cell types in vivo and in vitro. These capsids can be used for various gene therapy applications targeting pancreatic islets, as well as other tissues, such as the liver, which is relevant for other diseases. The selected AAV vectors, and hence, the capsids, also had increased tropism for liver cells. Among the selected chimeric capsids is one designated AAV-KP1 (SEQ ID NO:418). It facilitates transduction of primary human islet cells and human embryonic stem cell-derived β cells with up to 10-fold higher efficiency compared with previously studied best-in-class AAV vectors. This chimeric capsid also transduces mouse and human hepatocytes at very high levels in a humanized chimeric mouse model, thus providing a versatile vector for use in preclinical testing and human clinical trials, and ultimately therapy, for liver-based diseases or diseases for which gene expression in the liver is therapeutic. Among the selected chimeric capsids are those that also exhibit an enhanced neutralization profile as compared to a non-variant parent capsid polypeptide. Among these variant AAV capsid polypeptides are those that exhibit enhanced neutralization profile against pooled human immunoglobulins compared to a non-variant parent capsid polypeptide. These identified and selected capsids includes those designated KP1, KP2, and KP3, whose protein sequences are set forth in SEQ ID NOs:418-420, encoded by nucleic acid molecules whose sequences are set forth in SEQ ID NOs: 421-423, respectively. Particulars of exemplary methods used to generate and screen for the capsids are described in Example 10.

There are several requisites for effective gene therapy for hemophilia that must be satisfied; the gene therapy vectors and encoded modified FIX polypeptides provided herein satisfy these criteria. Gene therapy for hemophilia should provide sustained clotting factor activity to normalize the phenotype. Normal clotting levels are achieved by activity of at least 40% or 50% activity; normal clotting levels are a goal of gene therapy. Achieving lower levels, such as at least about 12% (or 10%) up to 40% or 50%, provides protection from spontaneous hemarthrosis, and also can be a goal. Even providing sufficient clotting activity (about 5% to 10%) to reduce annual bleeds, from >30 (severe hemophilia), to about 15-20 (moderate), improves quality of life. The gene therapy vectors, which effectively target and transduce hepatocytes, and which encode the modified FIX polypeptides as provided herein, provide high levels of expression of the modified FIX polypeptides, which are at least about 7-10-fold more potent than wild-type FIX. As a result, the doses of the vectors can be substantially lower (at least about 5-, 10- or more fold lower), than prior vectors. Consequently this reduces toxicity, including immunogenicity and inflammatory reactions. The combination of the AAV vectors described herein and the modified FIX polypeptides results in clinically relevant levels of FIX and also reduces viral load, thereby reducing immunogenicity and liver toxicity, compared to other vectors. To achieve expression in the liver from the AAV vectors, the nucleic acid encoding modified FIX provided herein includes an intron, generally a portion of the first intron, following the nucleic acid encoding the signal polypeptides. Dose dependent and stable FIX levels are achieved. The vectors, as exemplified encode the modified FIX with the partial (1.4 kb) intron following the nucleic acid encoding the intron, under control of liver-specific or liver recognized regulatory sequences, flanked by the AAV ITRs, and packaged in the chimeric capsids. As shown in the examples, stable FIX levels, as assessed by FIX antigen levels, are dose dependent and remain stable. These levels are achieved with lower doses than prior vectors, including DJ/8 (also referred to as DJ8). For example, a dose of 8×10¹⁰ viral genomes (vg)/kg, in a mouse study, achieved FIX levels of 20 U/ml, compared with levels of only 4 U/ml achieved with a dose of 2×10¹¹ vg/kg with DJ/8. To achieve the same activity with the Padua mutant (338L) in the same vector required a dosage of 7.4×10¹¹ vg/kg. Bleeding times also were reduced. Thus, the combination of the more potent FIX and the chimeric capsids provides for lower dosing and higher FIX activity.

### E. MODIFIED FIX POLYPEPTIDES

Modified factor IX polypeptides that can be encoded in the vectors are described in the following sections. In general, as described above, the vectors encode the full length precursor FIX and include the signal sequence, propeptide and mature portions. In the propeptide portion corresponding to residues 28-46 of SEQ ID NO:2, such as between amino acid residues corresponding to 28 and 29, an intron, such as a portion of a FIX intron, is inserted. The vector can include liver-specific regulatory sequences, such as liver-specific promoters and enhancers. These sequences are flanked by AAV ITRs. The FIX polypeptides can be modified by deletions, insertions or replacements (substitutions) of one or more amino acid residues in the primary sequence of a wild-type or unmodified FIX polypeptide. The resulting modified polypeptides exhibit improved properties or activities compared to the unmodified or wild-type FIX polypeptide. For example, the modified factor IX polypeptides, including modified FIXa polypeptides and fragments of modified factor IX and factor IXa polypeptides, can have altered post-translational modification, such as altered glycosylation, including hyperglycosylation, and/or altered phosphorylation or sulfation, such as decreased phosphorylation or sulfation; increased resistance to inhibitors, such as AT-III and/or heparin; decreased binding to LRP; increased catalytic activity; improved pharmacokinetic properties, including decreased clearance and increased serum half-life in vivo; increased coagulant activity; or any combination thereof. Typically, the modified FIX polypeptides exhibit procoagulant activity. FIX polypeptides described herein exhibit increased activity, increased resistance to endogenous inhibitors, such as antithrombin, and increased affinity for FVIIIa. For example, a mature FIX polypeptide that contains the replacements R318Y/R338E/T343R, such as the polypeptide of SEQ ID NO:394, or the same polypeptide in which residue 148 is A (alanine) has about 2.5-fold increased Factor X activation, 21-fold increased resistance to inhibition by antithrombin, and about 8-fold increase in FVIIIa affinity compared to wild-type or BeneFIX® FIX. The combination of these properties provides about 22-fold increase in potency. Exemplary modified FIX polypeptides with these properties are described in the Examples and elsewhere.

Thus, provided herein are modified FIX polypeptides that exhibit increased coagulant activity upon activation from their single-chain zymogen form and subsequent binding to the cofactor, FVIIIa. Such modified FIX polypeptides can be administered to patients with diseases or conditions characterized by insufficient coagulation, such as, for example, hemophilia B. Nucleic acid encoding other modified FIX known to those of skill in the art that have increased potency also can be packaged in the AAV vectors provided herein and as described herein, including inclusion of all or part of an intron.

In some examples, the modified FIX polypeptides provided herein exhibit increased resistance to inhibitors, including AT-III, heparin and the AT-III/heparin complex, compared to an unmodified FIX polypeptide. Such modified FIX polypeptides can exhibit increased coagulant activity compared to an unmodified FIX polypeptide. In further examples, the modified factor IX polypeptides provided herein exhibit altered post-translational modification, such as altered glycosylation levels and/or altered types of glycosylation compared to an unmodified FIX polypeptide.

In some examples, the modified FIX polypeptides provided herein exhibit increased glycosylation compared to an unmodified FIX polypeptide. Thus, provided herein are hyperglycosylated FIX polypeptides. The modified FIX polypeptides can exhibit increased glycosylation by virtue of the incorporation of at least one non-native glycosylation site (i.e., a glycosylation site that is not found in the unmodified or wild-type FIX polypeptide) to which a carbohydrate moiety is linked. Such modified FIX polypeptides can exhibit improved pharmacokinetic properties in vivo, including decreased clearance and increased serum half-life. The introduction of a non-native glycosylation site and subsequent carbohydrate moiety can further improve the activity of the modified FIX polypeptide by sterically hindering the interaction of the FIX polypeptide with one or more other proteins. For example, a glycosylation site can be introduced such that when a carbohydrate moiety is attached at this site, it sterically hinders the interaction of the modified FIX polypeptide with the AT-III/heparin complex, resulting in a polypeptide with increased resistance to AT-III/heparin. This can further reduce clearance of the polypeptide from the circulation. Thus, the effects of the introduction of a new glycosylation site can be several-fold if the carbohydrate moiety also sterically hinders an interaction with another protein(s), such as the AT-III/heparin complex.

For example, the modified FIX polypeptides provided herein can contain one or more modifications that introduce one or more non-native glycosylation sites compared to the unmodified FIX polypeptide. For example, 1, 2, 3, 4, 5, 6, or more non-native glycosylation sites can be introduced. Glycosylation sites that can be introduced include, but are not limited to, N-glycosylation sites, O-glycosylation sites, or a combination thereof. Thus, when produced in a cell that facilitates glycosylation, or following in vitro glycosylation, the modified FIX polypeptides provided herein can contain 1, 2, 3, 4, 5, 6 or more carbohydrate moieties, each linked to different non-native glycosylation sites, in addition to the carbohydrate moieties linked to the native glycosylation sites (e.g., the native glycosylation sites corresponding to S53, S61, N157, N167, T159, T169, T172 and T179 of the mature FIX polypeptide set forth in SEQ ID NO:3). In a particular example, the modified FIX polypeptides provided herein contain one or more non-native N-glycosylation sites. Thus, the modified FIX polypeptides can exhibit increased levels of N-glycosylation compared to an unmodified FIX polypeptide.

The modified FIX polypeptides with increased glycosylation also can exhibit, for example, increased solubility, increased AT-III/heparin resistance, increased serum half-life, decreased immunogenicity and/or increased coagulant activity compared to an unmodified FIX polypeptide. Such modified FIX polypeptides can be used in the treatment of bleeding disorders or events, such as hemophilias or injury, where the FIX polypeptides can function to promote blood coagulation. In some instances, the modified FIX polypeptides provided herein that exhibit increased glycosylation also can contain one or more modifications that render the protein inactive, or mostly inactive. Such polypeptides, therefore, can exhibit increased anti-coagulant activity and can be used in the treatment of thrombotic events, conditions or diseases. Typically, however, the modified FIX polypeptides provided herein are procoagulants.

The modified FIX polypeptides provided herein also can exhibit other activities and/or properties. For example, some of the modified FIX polypeptides contain one or more modifications that increase catalytic activity. In other examples, the modified FIX polypeptides contain one or more modifications that decrease phosphorylation, sulfation, hydroxylation and/or glycosylation. In further examples, the modified FIX polypeptides contain modifications that interfere with the interaction between FIX and LRP. By interrupting the binding of FIX to LRP, the clearance of FIX from circulation can be decreased. Hence, modifications that reduce the binding of FIX to LRP can improve the pharmacokinetic properties of FIX in vivo.

The modifications, such as amino acid replacements, described herein, such as those modifications that introduce one or more non-native glycosylation sites or increase resistance to inhibitors, can be made in any FIX polypeptide (e.g., unmodified or wild-type FIX polypeptide), including a precursor FIX polypeptide with a sequence set forth in SEQ ID NO:2, a mature FIX polypeptide set forth in SEQ ID NO:3, or in a FIX polypeptide having a sequence of amino acids that exhibits at least 40%, 50%, 60%, 70%, 80%, 90%, 91%, 92%, 93%, 94%, 95%, 96%, 97%, 98%, or 99% sequence identity to the FIX polypeptide set forth in SEQ ID NOs:2 or SEQ ID NO:3. It is understood that reference herein to amino acid residues is with respect to the numbering of the mature FIX polypeptide set forth in SEQ ID NO:3. It is within the level of one of skill in the art to identify a corresponding amino acid residue in another FIX polypeptide of any form, such as a precursor, mature or other active form, by alignment of the sequence of the other FIX polypeptide with SEQ ID NO:3 (see, e.g., FIGS. 3A-D). Any amino acid replacement provided herein can be made at a corresponding amino acid residue that differs or is not the same as the replacement amino acid residue. It is within the level of one of skill in the art to test any resulting modified FIX polypeptide for activity or property as described herein.

For example, the modifications, such as an amino acid replacement, can be made in any species, allelic or modified variant, such as those described in the art. Allelic variants of FIX include, but are not limited to, T148A and T412P. Any of the amino acid replacements provided herein can be a Factor IX that contains mutations T148A or T412P. For example, the modifications such as any amino acid replacement, can be made in a FIX polypeptide set forth in SEQ ID NO:325 or SEQ ID NO:20. Exemplary species variants for modification herein include, but are not limited to, human and non-human polypeptides including FIX polypeptides from chimpanzee, rhesus macaque, mouse, rat, guinea pig, pig, dog, cat, rabbit, chicken, cow, sheep, frog, zebrafish and Japanese pufferfish FIX polypeptides, whose sequences are set forth in SEQ ID NOs: 4-18, respectively. Modifications in a FIX polypeptide can be made to a FIX polypeptide that also contains other modifications, such as those described in the art, including modifications of the primary sequence and modifications not in the primary sequence of the polypeptide (see, e.g., Section D, which describes exemplary modified FIX polypeptides to which the amino modifications described herein can be made).

In other examples, the modifications, such as an amino acid replacement, can be made in any active fragment of a FIX polypeptide, such as an active fragment of SEQ ID NO:2 or SEQ ID NO:3, or an active fragment of a species, allelic or modified variant, such as those described in the art. The active fragment contains a contiguous sequence of amino acids containing the catalytically active domain of the polypeptide or a catalytically active portion thereof containing the amino acid modifications, such as amino acid replacements describes herein. The active fragment exhibit at least 30%, 40%, 50%, 60%, 70%, 80%, 90%, 95% or more of the activity of the mature form of the polypeptide, such as the FIX polypeptide set forth in SEQ ID NO:3.

Modification of FIX polypeptides also include modification of polypeptides that are hybrids of different FIX polypeptides and also synthetic FIX polypeptides prepared recombinantly or synthesized or constructed by other methods known in the art based upon the sequence of known polypeptides. For example, based on alignment of FIX with other coagulation factor family members, including, but not limited to, Factor FVII (FVII) and Factor X (FX), homologous domains among the family members are readily identified. Chimeric variants of FIX polypeptides can be constructed where one or more amino acids or entire domains are replaced in the FIX amino acid sequence using the amino acid sequence of the corresponding family member. Additionally, chimeric FIX polypeptides include those where one or more amino acids or entire domains are replaced in the human FIX amino acid sequence using the amino acid sequence of a different species. Such chimeric proteins can be used as the starting, unmodified FIX polypeptide herein.

Modifications provided herein of a starting, unmodified reference polypeptide include amino acid replacements or substitution, additions or deletions of amino acids, or any combination thereof. For example, modified FIX polypeptides include those with 1, 2, 3, 4, 5, 6, 7, 8, 9, 10, 11, 12, 13, 14, 15, 16, 17, 18, 19, 20, 30, 40, 50 or more modified positions. In some examples, a modification that is made to alter one activity or property of FIX also can, or instead, affect one more other activities or properties. For example, a modification made to increase resistance to inhibitors also, or instead, can increase catalytic activity. In another example, a modification made to introduce a new glycosylation site also can result in increased resistance to inhibitors and/or increased catalytic activity. In a further example, a modification made to decrease binding to LRP can also, or instead, increase resistance to an inhibitor, such as AT-III/heparin. Thus, although the modifications described herein typically are described in relation to their intended effect on FIX activities or properties, it is understood that any of the modifications described herein, alone or in conjunction with one or more other modifications, can result in changes in other, unpredicted, activities or properties.

Any modification provided herein can be combined with any other modification known to one of skill in the art. Typically, the resulting modified FIX polypeptide exhibits increased coagulation activity when it is in its two-chain form. The activities or properties that can be altered as a result of modification include, but are not limited to, coagulation or coagulant activity; pro-coagulant activity; proteolytic or catalytic activity such as to effect Factor X (FX) activation; antigenicity (ability to bind to or compete with a polypeptide for binding to an anti-FIX antibody); ability to bind FVIIIa, antithrombin III, heparin and/or factor X; ability to bind to phospholipids; three-dimensional structure; pI; and/or conformation. Included among the modified FIX polypeptides provided herein are those that have increased resistance to antithrombin III (AT-III), increased resistance to heparin, altered glycosylation, such as increased glycosylation, increased catalytic activity, and improved pharmacokinetic properties, such as i) decreased clearance, ii) altered volume of distribution, iii) enhanced in vivo recovery, iv) enhanced total protein exposure in vivo (i.e., AUC), v) increased serum half-life (α-, β-, and/or γ-phase), and/or vi) increased mean resonance time (MRT).

In some examples, a modification can affect two or more properties or activities of a FIX polypeptide. For example, a modification can result in increased AT-III resistance and increased catalytic activity of the modified FIX polypeptide compared to an unmodified FIX polypeptide. In another example, a modification that introduces a non-native N-glycosylation site and, thus, can increase the glycosylation levels of the polypeptide when expressed in an appropriate cell, such as a mammalian cell, also can result in increased catalytic activity of the modified FIX polypeptide compared to an unmodified FIX polypeptide. Modified FIX polypeptides provided herein can be assayed for each property and activity to identify the range of effects of a modification. Such assays are known in the art and described below. Typically, changes to the properties and/or activities of the modified FIX polypeptides provided herein are made while retaining other FIX activities or properties, such as, but not limited to, binding to FVIIIa and/or binding and activation of FX. Hence, modified FIX polypeptides provided herein retain FVIIIa binding and/or FX binding and activation as compared to a wild-type or starting form of the FIX polypeptide. Typically, such activity is substantially unchanged (less than 1%, 5% or 10% changed) compared to a wild-type or starting protein. In other examples, the activity of a modified FIX polypeptide is increased or is decreased as compared to a wild-type or starting FIX polypeptide. Activity can be assessed in vitro or in vivo and can be compared to the unmodified FIX polypeptide, such as for example, the mature, wild-type native FIX polypeptide (SEQ ID NO:3), the wild-type precursor FIX polypeptide (SEQ ID NO:2), or any other FIX polypeptide known to one of skill in the art that is used as the starting material.

The modifications provided herein can be made by standard recombinant DNA techniques such as are routine to one of skill in the art. Any method known in the art to effect mutation of any one or more amino acids in a target protein can be employed. Methods include standard site-directed mutagenesis of encoding nucleic acid molecules, or by solid phase polypeptide synthesis methods.

Other modifications that are or are not in the primary sequence of the polypeptide also can be included in a modified FIX polypeptide, or conjugate thereof, including, but not limited to, the addition of a carbohydrate moiety, the addition of a polyethylene glycol (PEG) moiety, the addition of an Fc domain, a serum albumin and/or other protein. For example, such additional modifications can be made to increase the stability or half-life of the protein.

The resulting modified FIX polypeptides include those that are single-chain zymogen polypeptides and those that are two-chain zymogen-like polypeptides (i.e., FIXa polypeptides that are not bound to the cofactor, FVIIIa). Any modified FIX polypeptide provided herein that is a single-chain polypeptide can be activated to generate a modified FIXa (i.e., a two-chain form). The activities of a modified FIX polypeptide are typically exhibited in its two-chain form.

1. Exemplary Amino Acid Replacements

Described herein are modified FIX polypeptides for use in the prophylactic subcutaneous methods and regimens provided herein. The FIX polypeptides contain one or more amino acid replacements as described herein below with numbering of residues with respect to the numbering of SEQ ID NO:3. The same amino acid replacements can be made in corresponding amino acid residues in another FIX polypeptide (see, e.g., FIG. 3 for exemplification of identification of corresponding amino acid residues). Corresponding residues are identified by alignment with the FIX of SEQ ID NO:3. The amino acid replacements confer altered glycosylation (e.g., by introduction of non-native glycosylation sites or elimination of native glycosylation sites), increased resistance to AT-III and/or heparin, increased catalytic activity, decreased LRP binding and/or alter posttranslational modifications. The resulting modified FIX polypeptides exhibit improved therapeutic efficacy, for example, due to improved pharmacodynamic or pharmacokinetic activity.

In particular, non-limiting examples of amino acid replacements in modified FIX polypeptides provided herein below are at any one or more amino acid residues 155, 318, 338, 343, 403 and/or 410 with numbering with respect to the mature FIX polypeptide set forth in SEQ ID NO:3 (corresponding to amino acid residues [155], 150, 170, 175, 233 and/or 240, respectively, by chymotrypsin numbering). The residues corresponding to any of 155, 318, 338, 343, 403 and/or 410 in other FIX polypeptides can be determined by sequence alignment with SEQ ID NO:3 (see, e.g., FIGS. 3A-3D). It is understood that the amino acid replacements provided herein at any of amino acid residues 155, 318, 338, 343, 403 and/or 410 with numbering with respect to SEQ ID NO:3 can be made in other FIX polypeptides as described elsewhere herein. It is also understood that residues corresponding to any of the other amino acid replacements provided herein also can be identified in other FIX polypeptides as exemplified herein (see, e.g., FIGS. 3A-3D).

In particular, the FIX polypeptides for use in the methods and regimens provided herein are amino acid replacement of tyrosine at amino acid residue Y155 (Y155F), Y155L, Y155H, R318A, R318Y, R318E, R318F, R318W, R318D, R318I, R318K, R318L, R318M, R318N, R318S, R318V, R318Y, R338A, R338E, T343R, T343E, T343D, T343F, T343I, T343K, T343L, T343M, T343Q, T343S, T343V, T343W, T343Y, R403A, R403E, E410Q, E410S, E410N, E410A, E410D, or a conservative amino acid replacement (see, e.g., Table 4). In some examples, the amino acid replacement is Y155F, R318Y, R318E, R338E, T343R, R403E and/or E410N or conservative amino acid replacements thereof.

For example, as shown by the data herein, amino acid replacement at position R318 with reference to SEQ ID NO:3 (150 by chymotrypsin numbering) confers resistance to inhibition by the AT-III/heparin complex. An amino acid replacement at position R338 (R170 by chymotrypsin numbering) also confers resistance to inhibition by the AT-III/heparin complex. In this respect, the amino acid position R338 is the site of a natural mutation (R170L) that has been reported to exhibit 5-10 fold enhanced clotting activity in an in vitro clotting assay (International Application Pub. No. WO 2010/029178). The assay as described was performed with conditioned media rather than purified protein and the protein concentration was measured using an ELISA assay. Consequently, these data could reflect a higher fraction of active material in the R338L (R170L) preparation as compared to the wild-type comparator preparation or a higher level of contaminants that are active in a clotting assay. Nevertheless, as shown herein, there is a 3.5- to 4-fold increased efficiency for FX activation by variants containing A, E and L at position 338 (170). As found herein, the R338E mutation, in addition, exhibited an approximately 88-fold resistance to inhibition by the heparin/AT-III complex as well as 2-fold enhanced binding to the co-factor, FVIIIa.

A 4 amino acid thrombin loop swap mutation into FIX, from positions 342-345 (174-177 by chymotrypsin numbering) has been reported to reduce the binding of FIXa to sLRP (see, Rohlena et al., (2003) J. Biol. Chem. 9394-9401). Mutation of the residue at position T343 (T175 by chymotrypsin numbering) did not confer any significant effect on the pharmacokinetic (PK) properties of FIX. It is found herein that the mutation T343R (T175R by chymotrypsin numbering), however, increases the catalytic efficacy for activation of FX by a factor of about 3.1, produces an approximately 5.6-fold resistance to the heparin/AT-III complex, and increases the affinity for FVIIIa by a factor of approximately 1.6-fold.

Also as shown herein, mutations at position R403 (R233 by chymotrypsin numbering) confer resistance to inhibition by the heparin/AT-III complex. Mutations at position E410 (E240 by chymotrypsin numbering), such as E410N, produce a significant, heretofore unobserved, 1.3- to 2.8-fold increase in the catalytic efficacy for activation of FX.

Also, as shown therein, there is a synergy in mutations at R338 and T343 (R170 and T175 by chymotrypsin numbering), particularly between R338E and T343R in enhanced binding to the co-factor FVIII. Synergy also was observed between mutations at positions R338 and E410 (R170 and E240 by chymotrypsin numbering), particularly R338E and E410N. The two double mutants, exemplified herein, R338E/T343R and R338E/E410N exhibit 24- to 28-fold improved binding to FVIIIa while each of the single mutations alone enhance binding by 1.6-2.2-fold each.

Other exemplary amino acid replacements in a FIX polypeptide provided herein found to confer an altered property or activity as described below can be at any amino acid residue from among 1, 5, 53, 61, 64, 85, 103, 104, 105, 106, 108, 148, 157, 158, 159, 167, 169, 172, 179, 202, 203, 204, 205, 228, 239, 241, 243, 247, 249, 251, 257, 259, 260, 262, 284, 293, 312, 314, 315, 316, 317, 319, 320, 321, 333, 342, 345, 346, 392, 394, 400, 412, or 413, with reference to SEQ ID NO:3, or at a corresponding amino acid residue. For example, exemplary amino acid replacements in a FIX polypeptide provided herein also include, but are not limited to, Y1N, K5A, S53A, S61A, S61C, S61D, S61E, S61F, S61G, S61I, S61K, S61L, S61P, S61R, S61V, S61W, S61Y, D64A, D64C, D64F, D64H, D641, D64L, D64M, D64N, D64P, D64R, D64S, D64T, D64W, D85N, A103N, D104N, N105S, N105T, K106N, K106S, K106T, V108S, V108T, T148A, N157D, N157E, N157F, N157I, N157K, N157L, N157M, N157Q, N157R, N157V, N157W, N157Y, S158A, S158D, S158E, S158F, S158G, S158I, S158K, S158L, S158M, S158R, S158V, S158W, S158Y, T159A, N167D, N167Q, N167E, N167F, N167G, N167H, N167I, N167K, N167L, N167M, N167P, N167R, N167V, N167W, N167Y, T169A, T169D, T169E, T169F, T169G, T169I, T169K, T169L, T169M, T169P, T169R, T169S, T169V, T169W, T169Y, T172A, T172D, T172E, T172F, T172G, T172I, T172K, T172L, T172M, T172P, T172R, T172S, T172V, T172W, T172Y, T179A, V202M, V202Y, D203N, D203M, D203Y, D203F, D203H, D203I, D203K, D203L, D203R, D203V, D203W, A204M, A204Y, A204F, A204I, A204W, F205S, F205T, K228N, E239A, E239S, E239R, E239K, E239D, E239F, E239I, E239L, E239M, E239N, E239T, E239V, E239W, E239Y, T241N, H243S, H243T, K247N, N249S, N249T, I251S, H257F, H257E, H257D, H257I, H257K, H257L, H257M, H257Q, H257R, H257S, H257V, H257W, H257Y, N260S, A262S, A262T, Y284N, K293E, K293A, R312A, R312Y, R312L, R312C, R312D, R312E, R312F, R312I, R312K, R312L, R312M, R312P, R312Q, R312S, R312T, R312V, R312W, R312Y, F314N, H315S, K316M, K316D, K316F, K316H, K316I, K316L, K316M, K316R, K316S, K316T, K316V, K316W, K316Y, G317N, S319N, A320S, L321N, L321S, L321T, R333A, R333E, F342I, F342D, F342E, F342K, F342L, F342M, F342S, F342T, F342V, F342W, F342Y, Y345A, Y345T, N346D, N346Y, N346E, N346F, N346H, N346I, N346K, N346L, N346M, N346Q, N346R, N346T, N346V, N346W, K392N, K394S, K394T, K400A, K400E, K400C, K400D, K400F, K400G, K400L, K400M, K400P, K400S, K400T, K400V, K400Y, T412A, T412V, T412C, T412D, T412E, T412F, T412G, T412I, T412M, T412P, T412W, T412Y, and K413N, in a mature FIX polypeptide having a sequence set forth in SEQ ID NO:3, or the same replacement in a corresponding amino acid residue position.

For example, exemplary properties and activities that are altered by the modifications (e.g., amino acid replacements) provided herein are described as follows.

a. Altered Glycosylation

The modified Factor IX polypeptides provided herein can exhibit altered glycosylation levels and/or altered types of glycosylation compared to an unmodified FIX polypeptide. In some examples, the modified FIX polypeptides provided herein exhibit increased glycosylation compared to an unmodified FIX polypeptide. Thus, among the modified FIX polypeptides described herein are hyperglycosylated FIX polypeptides.

i. Advantages of Glycosylation

Many mammalian proteins are glycosylated with variable numbers of carbohydrate chains, each of which can have differing carbohydrate structures. Such carbohydrates can have an important role in the stability, solubility, activity, serum half-life and immunogenicity of the protein. Thus, the properties and activities of a protein can be altered by modulating the amount and/or type of glycosylation. For example, glycosylation can increase serum-half-life of polypeptides by increasing the stability, solubility, and reducing the immunogenicity of a protein. This is of particular interest for therapeutic polypeptides, where increased solubility, serum half-life and stability of the therapeutic polypeptide can result in increased therapeutic efficacy.

Oligosaccharides are important in intra- and inter-cell events such as a recognition, signaling and adhesion. Carbohydrates also assist in the folding of secreted proteins. Glycosylation sites provide a site for attachment of monosaccharides and oligosaccharides to a polypeptide via a glycosidic linkage, such that when the polypeptide is produced, for example, in a eukaryotic cell capable of glycosylation, it is glycosylated. There are several types of protein glycosylation. N-linked and O-linked glycosylation are the major classes, in which an asparagine residue, or a serine or threonine residue, respectively, is modified. Other types of glycans include glycosaminoglycans and glycosylphophatidylinositol (GPI)-anchors. Glycosaminoglycans are attached to the hydroxy oxygen of serine, while GPI anchors attach a protein to a hydrophobic lipid anchor, via a glycan chain. C-glycosylation also can occur at the consensus sequence Trp-X-X-Trp, where the indol side chain of the first tryptophan residue in the sequences is modified with an α-mannopyranosyl group (Furmanek et al., (2000) Acta Biochim. Pol. 47:781-789).

The presence of a potential glycosylation site does not, however, ensure that the site will be glycosylated during post-translational processing in the ER. The level of glycosylation can vary at any given site, as can the glycan structures. The differences in levels and types of glycosylation at particular sites can be attributed, at least in part, to the sequence context and secondary structure around the potential glycosylation site.

O-linked glycosylation involves the attachment of the sugar units, such as N-acetylgalactosamine, via the hydroxyl group of serine, threonine, hydroxylysine or hydroxyproline residues. It is initiated by the attachment of one monosaccharide, following which others are added to form a mature O-glycan structure. There is no known motif for O-glycosylation, although O-glycosylation is more probable in sequences with a high proportion of serine, threonine and proline residues. Further, secondary structural elements such as an extended β turn also may promote O-glycosylation. O-glycosylation lacks a common core structure. Instead, several types of glycans can be attached at the selected O-glycosylation sites, including O—N-acetylgalactosamine (O-GalNAc), O—N-acetylglucosamine (O-GlcNAc), O-fucose and O-glucose.

In contrast to O-glycosylation, the N-linked glycosylation consensus sequence motif is well characterized. During N-linked glycosylation, a 14-residue oligosaccharide is transferred to the asparagine residue in the Asn-X-Ser/Thr/Cys consensus motif, where X is any amino acid except Pro. Glycosyltransferases then enzymatically trim the saccharide and attach additional sugar units to the mannose residues. The sequence adjacent to the consensus motif also can affect whether or not glycosylation occurs at the consensus sequence. Thus, the presence of the Asn-X-Ser/Thr/Cys consensus sequence is required but not necessarily sufficient for N-linked glycosylation to occur. In some instances, changes to the adjacent sequence results in glycosylation at the consensus motif where there previously was none (Elliot et al., (2004) J. Biol. Chem. 279:16854-16862).

N-linked oligosaccharides share a common core structure of GlcNAc₂Man₃. There are three major types of N-linked saccharides in mammals: high-mannose oligosaccharides, complex oligosaccharides and hybrid oligosaccharides. High-mannose oligosaccharides essentially contain two N-acetylglucosamines with several mannose residues. In some instances, the final N-linked high-mannose oligosaccharide contains as many mannose residues as the precursor oligosaccharide before it is attached to the protein. Complex oligosaccharides can contain almost any number of mannose, N-acetylglucosamines and fucose saccharides, including more than the two N-acetylglucosamines in the core structure.

Glycosylation can increase the stability of proteins by reducing the proteolysis of the protein and can protect the protein from thermal degradation, exposure to denaturing agents, damage by oxygen free radicals, and changes in pH. Glycosylation also can allow the target protein to evade clearance mechanisms that can involve binding to other proteins, including cell surface receptors. The sialic acid component of carbohydrate in particular can enhance the serum half-life of proteins. Sialic acid moieties are highly hydrophilic and can shield hydrophobic residues of the target protein. This increases solubility and decreases aggregation and precipitation of the protein. Decreased aggregation reduces the likelihood of an immune response being raised to the protein. Further, carbohydrates can shield immunogenic sequences from the immune system, and the volume of space occupied by the carbohydrate moieties can decrease the available surface area that is surveyed by the immune system. These properties can lead to the reduction in immunogenicity of the target protein.

Modifying the level and/or type of glycosylation of a therapeutic polypeptide can affect the in vivo activity of the polypeptide. By increasing the level of glycosylation, recombinant polypeptides can be made more stable with increased serum half-life, reduced serum clearance and reduced immunogenicity. This can increase the in vivo activity of the polypeptide, resulting in reduced doses and/or frequency of dosing to achieve a comparable therapeutic effect. For example, a hyperglycosylated form of recombinant human erythropoietin (rHuEPO), called Darbepoetin alfa (DA), has increased in vivo activity and prolonged duration of action. The increased carbohydrate and sialic acid content of the hyperglycosylated DA polypeptide results in a serum half-life that is three times greater than that of the unmodified rHuEPO. This increased serum half-life results in increased bioavailability and reduced clearance, which can allow for less frequent dosing and/or lower dosages, with associated increased convenience for the patient, reduced risk of adverse effects and improved patient compliance.

ii. Exemplary Modified FIX Polypeptides with Altered Glycosylation

Provided herein are modified FIX polypeptides that are modified to exhibit altered glycosylation compared to an unmodified FIX polypeptide. The modified FIX polypeptides can exhibit increased or decreased glycosylation, such as by the incorporation of non-native glycosylation sites or the deletion of native glycosylation sites, respectively. For example, the modified FIX polypeptides can contain 1, 2, 3, 4 or more non-native N-glycosylation sites. The non-native N-glycosylation sites can be introduced by amino acid replacement(s) (or substitution(s)), insertion(s) or deletion(s), or any combination thereof, wherein the amino acid replacement(s), insertion(s) and/or deletion(s) result in the establishment of the glycosylation motif Asn-Xaa-Ser/Thr/Cys, where Xaa is not proline. In other examples, the modified FIX polypeptides provided herein can have a reduced number of glycosylation sites compared to an unmodified FIX polypeptide, typically resulting in a reduced level of glycosylation compared to the unmodified FIX polypeptide. In further examples, the modified FIX polypeptides exhibit the same levels of glycosylation as wild-type FIX, but exhibit different types of glycosylation. For example, a modified FIX polypeptide can exhibit the same number of glycosylation sites and the same level of glycosylation as an unmodified FIX polypeptide, but can have different types of glycosylation, such as, for example, different relative amounts of N- and O-glycosylation compared to an unmodified FIX polypeptide.

(a) Introduction of Non-Native Glycosylation Site(s)

In particular examples, a non-native N-glycosylation site is introduced by amino acid replacement. In some instances, the creation of a non-native N-glycosylation site by amino acid replacement requires only one amino acid replacement. For example, if the unmodified FIX polypeptide contains a Gly-Ala-Ser sequence, then an N-glycosylation site can be created by a single amino acid substitution of the glycine with an asparagine, to create an Asn-Ala-Ser N-glycosylation motif. In another example, if the unmodified FIX polypeptide contains an Asn-Trp-Met sequence, then an N-glycosylation site can be created by a single amino acid substitution of the methionine with a cysteine (or threonine or serine). In other instances, the creation of a non-native N-glycosylation site by amino acid replacement requires more than one amino acid replacement. For example, if the unmodified FIX polypeptide contains a Gly-Arg-Phe sequence, then an N-glycosylation site can be created by two amino acid replacements: an amino acid substitution of the glycine with an asparagine, and an amino acid substitution of the phenylalanine with a cysteine (or threonine or serine), to create an Asn-Arg-Ser/Thr/Cys N-glycosylation motif. Thus, one of skill in the art can introduce one or more non-native N-glycosylation sites at any position in the FIX polypeptide.

The position at which a non-native glycosylation site is introduced into the FIX polypeptide to generate the modified FIX polypeptides provided herein is typically selected so that any carbohydrate moieties linked at that site do not adversely interfere with the structure, function and/or procoagulant activity of the FIX polypeptide, or that the amino acid modification(s) made to the polypeptide to introduce the non-native glycosylation site do not adversely interfere with the structure, function or activity of the FIX polypeptide. Thus, a non-native glycosylation site can be introduced into any position in a FIX polypeptide provided the resulting modified FIX polypeptide retains at least one activity of the wild type or unmodified FIX polypeptide. Conversely, one or more non-native glycosylation sites can be introduced into the modified FIX polypeptide at sites that may be involved in the interaction of FIX with an inhibitory molecule. The carbohydrate moiety that is linked to the new glycosylation site can sterically hinder the interaction between the inhibitory molecule and the modified FIX. Such steric hindrance can result in a modified FIX polypeptide with increased coagulant activity. For example, a carbohydrate moiety that is linked to a non-native glycosylation site contained in the modified FIX polypeptides provided herein can sterically hinder the interaction of the modified FIX with the AT-III/heparin complex. This can result in increased resistance of the modified FIX polypeptide to the inhibitory effects of AT-III/heparin.

Thus, a non-native glycosylation site can be introduced into the Gla domain, EGF1 domain, EGF2 domain, activation peptide and/or the protease domain, provided the resulting modified FIX polypeptide retains at least one activity of the wild type or unmodified FIX polypeptide. In other examples, a non-native glycosylation site is introduced into the EGF2 domain or the protease domain. The resulting modified FIX polypeptide retains at least one activity of the unmodified FIX polypeptide. In some examples, the modified FIX polypeptide retains at least 5%, 10%, 20%, 30%, 40%, 50%, 60%, 70%, 80%, 90%, 95% or more of the catalytic activity of the unmodified FIX polypeptide. In other examples, the modified FIX polypeptide retains at least 5%, 10%, 20%, 30%, 40%, 50%, 60%, 70%, 80%, 90%, 95% or more of the binding activity for FX of the unmodified FIX polypeptide. In other examples, the modified FIX polypeptide retains at least 5%, 10%, 20%, 30%, 40%, 50%, 60%, 70%, 80%, 90%, 95% or more of the binding activity for FVIIIa of the unmodified FIX polypeptide. In some assays and/or under some conditions, the modified FIX polypeptides can exhibit increased activity compared with the unmodified FIX protein (e.g., pharmacodynamic activity in vivo, and/or catalytic activity in the presence of ATIII/heparin or plasma).

Table 5 provides non-limiting examples of exemplary amino acid replacements, corresponding to amino acid positions of a mature FIX polypeptide as set forth in SEQ ID NO:3, that are included in a modified FIX polypeptide to increase glycosylation levels by introducing a non-native N-glycosylation site. In reference to such mutations, the first amino acid (one-letter abbreviation) corresponds to the amino acid that is replaced, the number corresponds to the position in the mature FIX polypeptide sequence with reference to SEQ ID NO:3, and the second amino acid (one-letter abbreviation) corresponds to the amino acid selected that replaces the first amino acid at that position. The amino acid positions for mutation also are referred to by the chymotrypsin numbering scheme where appropriate (i.e., when the mutation is located within the FIX protease domain). In instances where a modified amino acid position does not have a corresponding chymotrypsin number (i.e., is not within amino acid positions 181 to 415 corresponding to a mature FIX polypeptide set forth in SEQ ID NO:3, and is not set forth in Table 1, above), the position is denoted in brackets using mature FIX numbering. For example, A103N does not have a corresponding chymotrypsin number and is set forth as A[103]N when referring to chymotrypsin numbering. In Table 5 below, the sequence identifier (SEQ ID NO) is identified in which exemplary amino acid sequences of the modified FIX polypeptide are set forth. Also identified in Table 5 are the positions of the non-native glycosylation sites generated by the modifications.

In some instances, only one amino acid replacement is required to create a non-native N-glycosylation site. For example, the aspartic acid (Asp, D) at position 85 (corresponding to the mature FIX polypeptide set forth in SEQ ID NO:3) can be replaced with an asparagine (Asn, N) to generate a non-native glycosylation site in the EGF2 domain at amino acid position 85 in the resulting modified FIX polypeptide. In another example, the isoleucine (Ile, I) at position 251 (corresponding to the mature FIX polypeptide set forth in SEQ ID NO:3) can be replaced with a serine (Ser, S) to generate a non-native N-glycosylation site in the protease domain at amino acid position 249 in the resulting modified FIX polypeptide. In other instances, two amino acid replacements are required to create a new glycosylation site. For example, the alanine (Ala, A) at position 103 (based on numbering of a mature FIX set forth in SEQ ID NO:3) can be replaced with an asparagine (Asn, N), and the asparagine at position 105 can be replaced with a serine (Ser, S) to create a non-native N-glycosylation site in the EGF2 domain at amino acid position 103 in the resulting modified FIX polypeptide. In another example, the threonine (Thr, T) at position 241 is replaced with an asparagine and the histidine (His, H) at position 243 is replaced with a serine to create a non-native N-glycosylation site in the protease domain at amino acid position 243.

TABLE 5

| | | Non-native | Non-native | |
|---|---|---|---|---|
| | | glycosylation | glycosylation | |
| Modification | Modification | site | site | SEQ |
| (mature FIX | (chymotrypsin | (mature FIX | (chymotrypsin | ID |
| numbering) | numbering) | numbering) | numbering) | NO. |
| A103N/N105S | A[103]N/N[105]S | N103 | N[103] | 77 |
| D104N/K106S | D[104]N/K[106]S | N104 | N[104] | 78 |
| K106N/V108S | K[106]N/V[108]S | N106 | N[106] | 79 |
| D85N | D[85]N | N85 | N[85] | 80 |
| D203N/F205T | D39N/F41T | N203 | N39 | 99 |
| K228N | K63N | N228 | N63 | 101 |
| I251S | I86S | N249 | N84 | 103 |
| A262S | A95bS | N260 | N95 | 106 |
| K413N | K243N | N413 | N243 | 107 |
| E410N | E240N | N410 | N240 | 108 |
| E239N | E74N | N239 | N74 | 109 |
| T241N/H243S | T76N/H78S | N241 | N76 | 110 |
| K247N/N249S | K82N/N84S | N247 | N82 | 111 |
| L321N | L153N | N321 | N153 | 112 |
| K392N/K394S | K222N/K224S | N392 | N222 | 114 |
| N260S | N95S | N258 | N93 | 116 |
| S319N/L321S | S151N/L153S | N319 | N151 | 115 |
| Y284N | Y117N | N284 | N117 | 117 |
| G317N | G149N | N317 | N149 | 118 |
| R318N/A320S | R150N/A152S | N318 | N150 | 119 |
| F314N/K316S | F145N/K148S | N314 | N145 | 177 |

The modified FIX polypeptides provided herein can contain modifications that result in the introduction of two or more non-native N-glycosylation sites. For example, the modifications set forth in Table 5 can be combined, resulting in a modified FIX polypeptide that contains 2, 3, 4, 5, 6 or more non-native N-glycosylation sites. Any two or more of the modifications set forth in Table 5 can be combined. For example, included among the modified FIX polypeptides provided herein are modified FIX polypeptides that contain the amino acid substitutions D104N/K106S/K228N, resulting in a FIX polypeptide with two non-native glycosylation sites at amino acid positions 104 and 228, respectively (numbering corresponding to the mature FIX polypeptide set forth in SEQ ID NO:3). In another example, a modified FIX polypeptide can contain amino acid substitutions D85N/K247N/N249S/K392N/K394S, resulting in a FIX polypeptide with three non-native glycosylation sites at amino acid positions 85, 247 and 392, respectively (numbering corresponding to the mature FIX polypeptide set forth in SEQ ID NO:3). Table 6 sets forth exemplary FIX polypeptides having two or more non-native N-glycosylation sites.

TABLE 6

| | | Non-native | Non-native | |
|---|---|---|---|---|
| | | glycosylation | glycosylation | |
| Modifications | Modifications | site | site | SEQ |
| (mature FIX | (chymotrypsin | (mature FIX | (chymotrypsin | ID |
| numbering) | numbering) | numbering) | numbering) | NO. |
| D85N/I251S | D[85]N/I86S | N85 and N149 | N[85] and N84 | 104 |
| D85N/D203N/F205T | D[85]N/D39N/F41T | N85 and N203 | N[85] and N39 | 100 |
| D85N/K228N | D[85]N/K63N | N85 and N228 | N[85] and N63 | 102 |
| D85N/D104N/ | D[85]N/D[104N]/ | N85, N104 and | N[85], N[104] | 105 |
| K106S/I251S | K[106]6/I86S | N249 | and N84 | |
| A103N/N105S/ | A[103]N/N[105]S/ | N103 and N247 | N[103] and N82 | 178 |
| K247N/N249S | K82N/N84S | | | |
| D104N/K106S/ | D[104]N/K[106]S/ | N104 and N247 | N[104] and N82 | 179 |
| K247N/N249S | K82N/N84S | | | |
| K228N/I251S | K63N/I86S | N228 and N249 | N63 and N84 | 180 |
| A103N/N105S/I251S | A[103]N/N[105]S/I86S | N103 and N249 | N[103] and N84 | 181 |
| D104N/K106S/I251S | D[104]N/K[106]S/I86S | N104 and N249 | N[104] and N84 | 182 |
| K228N/K247N/N249S | K63N/K82N/N84S | N228 and N247 | N63 and N82 | 183 |
| K228N/K247N/N249S/ | K63N/K82N/N84S/ | N228, N247 | N63, N82 and | 184 |
| D104N/K106S | D[104]N/K[106]S | and N104, | N[104] | |
| D104N/K106S/N260S | D[104]N/K[106]S/N95S | N104 and N258 | N[104] and N93 | 185 |

The modified FIX polypeptides provided herein can contain one or more non-native glycosylation sites, such as one or more non-native N-glycosylation sites. Thus, when expressed in a cell that facilitates glycosylation, or when glycosylated using in vitro techniques well known in the art, the modified FIX polypeptides can exhibit increased levels of glycosylation compared to an unmodified FIX polypeptide. The level of glycosylation can be increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the level of glycosylation of unmodified or wild-type FIX polypeptide.

The modifications described herein to introduce one or more non-native glycosylation sites can be combined with any other mutation described herein or known in the art. Typically, the resulting modified FIX polypeptide exhibits increased coagulant activity compared to an unmodified FIX polypeptide. For example, one or more modifications that introduce one or more non-native glycosylation sites can be combined with modification(s) that increase resistance to an inhibitor, such as AT-III and/or heparin, increase catalytic activity, increase intrinsic activity, increase binding to phospholipids, decrease binding to LRP and/or improve pharmacokinetic and/or pharmacodynamic properties.

The modified FIX polypeptides provided herein that contain one or more non-native glycosylation sites and have altered glycosylation, such as increased levels of glycosylation, retain at least one activity of FIX, such as, for example, catalytic activity for its substrate, FX. Typically, the modified FIX polypeptides provided herein retain at least or at least about 5%, 10%, 20%, 30%, 40%, 50%, 60%, 70%, 80%, 90%, 95% or more of the catalytic activity exhibited by an unmodified FIX polypeptide. Increased levels of glycosylation can improve the pharmacokinetic properties of the modified FIX polypeptides by endowing the variant with one or more of the following properties: i) decreased clearance, ii) altered volume of distribution, iii) enhanced in vivo recovery, iv) enhanced total protein exposure in vivo (i.e., AUC), v) increased serum half-life (α, β, and/or γ phase), and/or vi) increased mean resonance time (MRT) compared to an unmodified FIX. The coagulant activity of the modified FIX polypeptides with altered glycosylation can be increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro.

(b) Elimination of Native Glycosylation Sites

The modified FIX polypeptides provided herein can have a reduced number of glycosylation sites compared to an unmodified FIX polypeptide. Typically, a reduction in the number of glycosylation sites results in a reduced level of glycosylation compared to the unmodified FIX polypeptide. The native glycosylation sites that can be removed include, for example, native N-glycosylation sites at amino acid positions corresponding to positions 157 and 167 of the mature FIX set forth in SEQ ID NO:3, and native O-glycosylation sites at amino acid positions corresponding to positions 53, 61, 159, 169, 172 and 179 of the mature FIX set forth in SEQ ID NO:3.

Any one or more native glycosylation sites can be removed by amino acid replacement(s), insertion(s) or deletion(s), or any combination thereof. For example, an amino acid replacement, deletion and/or insertion can be made to destroy the Asn/Xaa/Ser/Thr/Cys motif (where Xaa is not a proline), thereby removing an N-glycosylation site at position 157 or 167. In other examples, O-glycosylation sites are removed, such as by amino acid replacement or deletion of the serine residues at positions 53 or 61, or amino acid replacement or deletion of the threonine residues at positions 159 or 169. The resulting modified FIX polypeptide retains at least one activity of the unmodified FIX polypeptide. In some examples, the modified FIX polypeptide retains at least 5%, 10%, 20%, 30%, 40%, 50%, 60%, 70%, 80%, 90%, 95% or more of the catalytic activity of the unmodified FIX polypeptide. In other examples, the modified FIX polypeptide retains at least 5%, 10%, 20%, 30%, 40%, 50%, 60%, 70%, 80%, 90%, 95% or more of the binding activity for FX of the unmodified FIX polypeptide. In other examples, the modified FIX polypeptide retains at least 5%, 10%, 20%, 30%, 40%, 50%, 60%, 70%, 80%, 90%, 95% or more of the binding activity for FVIIIa of the unmodified FIX polypeptide. In some assays and/or under some conditions, the modified FIX polypeptides can exhibit enhanced properties compared with unmodified FIX (e.g., including but not limited to, increased in vivo recovery, increased AUC in vivo, and/or decreased clearance in vivo).

Table 7 provides non-limiting examples of exemplary amino acid replacements, corresponding to amino acid positions of a mature FIX polypeptide as set forth in SEQ ID NO:3, that are included in a modified FIX polypeptide to decrease glycosylation levels by removing or eliminating a native N-glycosylation site. In Table 7 below, the sequence identifier (SEQ ID NO) is identified in which exemplary amino acid sequences of the modified FIX polypeptide are set forth.

TABLE 7

| Mutation | Mutation | SEQ |
|---|---|---|
| (Mature FIX | (Chymotrypsin | ID |
| Numbering) | Numbering) | NO. |
| S53A | S[53]A | 88 |
| S61A | S[61]A | 87 |
| N157D | N[157]D | 75 |
| N157Q | N[157]Q | 98 |
| T159A | T[159]A | 89 |
| N167D | N[167]D | 85 |
| N167Q | N[167]Q | 86 |
| T169A | T[169]A | 90 |
| T172A | T[172]A | 91 |
| T179A | T[179]A | 92 |

The modifications described herein to eliminate one or more native glycosylation sites can be combined with any other mutation described herein or known in the art. Typically, the resulting modified FIX polypeptide exhibits increased coagulant activity compared to an unmodified FIX polypeptide. For example, one or more modifications that eliminate one or more native glycosylation sites can be combined with modification(s) that introduce a non-native glycosylation site, increase resistance to an inhibitor, such as AT-III and/or heparin, increase catalytic activity, increase intrinsic activity, increase binding to phospholipids, or improve pharmacokinetic and/or pharmacodynamic properties.

The modified FIX polypeptides provided herein that eliminate one or more native glycosylation sites retain at least one activity of FIX, such as, for example, catalytic activity for its substrate, FX. Typically, the modified FIX polypeptides provided herein retain at least or at least about 5%, 10%, 20%, 30%, 40%, 50%, 60%, 70%, 80%, 90%, 95% or more of the catalytic activity exhibited by an unmodified FIX polypeptide. In some instances, the coagulant activity of the modified FIX polypeptides with altered glycosylation can be increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro.

b. Increased Resistance to AT-III and Heparin

The activity of FIX can be inhibited by factors in the blood as part of the regulation of the coagulation process. Thus, provided herein are modified FIX polypeptides that exhibit increased resistance to the inhibitory effects of inhibitors, including AT-III and heparin. In some examples, the modified FIX polypeptides provided herein exhibit reduced binding affinity for heparin and/or a decreased second order rate constant for inhibition by AT-III alone and/or the AT-III/heparin complex. In further examples, the modified FIX polypeptides exhibit increased resistance to the AT-III alone, or heparin alone. Thus, provided herein are modified FIX polypeptides that exhibit increased resistance to AT-III, the AT-III/heparin complex and/or heparin.

i. AT-III

Antithrombin III (also known as antithrombin or AT-III) is an important anticoagulant serpin (serine protease inhibitor). AT-III is synthesized as a precursor protein containing 464 amino acid residues (SEQ ID NO:21). In the course of secretion a 32 residue signal peptide is cleaved to generate a 432 amino acid mature human antithrombin (SEQ ID NO:22). The 58 kDa AT-III glycoprotein circulates in the blood and functions as a serine protease inhibitor (serpin) to inhibit a large number of serine proteases of the coagulation system. The principal targets of AT-III are thrombin, factor Xa and factor IXa, although AT-III also has been shown to inhibit the activities of FXIa, FXIIa and, to a lesser extent, FVIIa.

The action of AT-III is greatly enhanced by glycosaminoglycans, such as the naturally occurring heparan sulfate or the various tissue-derived heparins that are widely used as anticoagulants in clinical practice. Unlike other serpins, which typically are effective without binding a secondary molecule, the reaction of AT-III in the absence of heparin with is target coagulations factors is unusually slow. In the absence of heparin, the reactive loop sequence of AT-III provides the determinants of the slow reactivity. Mutagenesis of the conserved P2-P1′ residues in the reactive loop center of AT-III, for example, affects the interaction of AT-III with proteases in the absence but not the presence of heparin.

AT-III binds in a highly specific manner to a unique pentasaccharide sequence in heparin that induces a conformational change in the reactive center loop. In such a conformation, the reactive center loop of AT-III can more efficiently interact with the reactive site of the serine protease, and effect inhibition. Evidence indicates that binding of heparin to AT-III generates new exosites that promote the interaction of FIXa, thrombin and FXa with AT-III. The tyrosine at position 253 and the glutamic acid at position 255, for example, have been shown to be key determinants of an exosite on AT-III that is generated by heparin binding, and that promotes the rapid, increased inhibition of FIXa by AT-III, compared to the inhibition observed with AT-III alone (Izaguirre et al., (2006) J. Biol. Chem. 281:13424-13432).

Mutational studies also have provided an indication of which residues in Factor IXa are involved in the interaction with AT-III/heparin. For example, modification of the arginine at position 318 of the mature FIX polypeptide (corresponding to position 150 by chymotrypsin numbering) reduces the reactivity of this mutant with AT-III/heparin by 33-fold to 70-fold (Yang, L. et al., (2003) J. Biol. Chem. 278(27):25032-8). The impairment of the reactivity between the FIXa mutant and AT-III is not as noticeable when AT-III is not bound to heparin, however, indicating that the interaction between the arginine at position 318 of the mature FIXa polypeptide and AT-III is effected when AT-III is in the heparin-activated conformation.

ii. Heparin

Heparin can inhibit the activity of FIXa in the intrinsic tenase complex in both an AT-III-dependent manner, as discussed above, and an AT-III-independent manner. Studies indicate that the AT-III-independent inhibition of FIXa activity by heparin is the result of oligosaccharide binding to an exosite on FIXa that disrupts the FVIIIa-FIXa interaction (Yuan et al., (2005) Biochem. 44:3615-3625; Misenheimer et al., (2007) Biochem. 46:7886-7895; Misenheimer et al. (2010) Biochem. 49:9977-10005). The heparin-binding exosite is in the Factor IXa protease domain, in an electropositive region extending from the arginine at position 338 (corresponding to position 170 by chymotrypsin numbering) to at least the arginine at position 403 (corresponding to position 233 by chymotrypsin numbering). This exosite overlaps with a region of FIXa that is critical to the interaction of FIXa with its cofactor, FVIIIa. Thus, binding of heparin to FIXa inhibits the interaction of FIXa with FVIIIa, thus reducing the intrinsic tenase activity.

iii. Exemplary FIX Polypeptides with Increased Resistance to AT-III and Heparin

Modifications can be made to a FIX polypeptide that increase its resistance to AT-III, heparin and/or the AT-III/heparin complex. Generally, such modified FIX polypeptides retain at least one activity of a FIX polypeptide. Typically, such modifications include one or more amino acid substitutions at any position of the FIX polypeptide that is involved in the interaction of FIXa with AT-III, heparin and/or the AT-III/heparin complex. Such modifications can, for example, result in a reduced rate of interaction of the modified FIXa polypeptide with AT-III alone, a reduced rate of interaction of the modified FIXa polypeptide to the AT-III/heparin complex, and/or a reduced binding affinity of the modified FIXa polypeptide for heparin alone. In some examples, the modification(s) introduces one or more non-native glycosylation sites. The carbohydrate moiety that is linked to the new glycosylation site can sterically hinder the interaction of the modified FIX with the AT-III/heparin complex, resulting in increased resistance of the modified FIX polypeptide to the inhibitory effects of AT-III/heparin. The modified FIXa polypeptides therefore exhibit increased resistance to the naturally inhibitory effects of AT-III, AT-III/heparin and/or heparin with respect to intrinsic tenase activity. When evaluated in an appropriate in vitro assay, or in vivo, such as following administration to a subject as a pro-coagulant therapeutic, the modified FIX polypeptides display increased coagulant activity as compared with unmodified FIX polypeptides.

As described herein below, one of skill in the art can empirically or rationally design modified FIXa polypeptides that display increased resistance to AT-III, AT-III/heparin and/or heparin. Such modified FIX polypeptides can be tested in assays known to one of skill in the art to determine if the modified FIX polypeptides display increased resistance to AT-III, AT-III/heparin and/or heparin. For example, the modified FIX polypeptides can be tested for binding to AT-III, AT-III/heparin and/or heparin. Generally, a modified FIX polypeptide that has increased resistance to AT-III, AT-III/heparin and/or heparin will exhibit decreased binding and/or decreased affinity for heparin and/or a decreased rate of interaction with AT-III and/or AT-III/heparin. Typically, such assays are performed with the activated form of FIX (FIXa), and in the presence or absence of the cofactor, FVIIIa, and phospholipids.

Provided herein are modified FIX polypeptides exhibiting increased resistance to AT-III, AT-III/heparin and/or heparin. FIX polypeptide variants provided herein have been modified at one or more of amino acid positions 202, 203, 204, 205, 228, 239, 257, 260, 293, 312, 316, 318, 319, 321, 333, 338, 342, 346, 400, 403, or 410 (corresponding to amino acid positions 38, 39, 40, 41, 63, 74, 92, 95, 126, 143, 145, 148, 150, 151, 153, 165, 170, 174, 178, 230, 233, and 240 respectively, by chymotrypsin numbering). These amino acid residues can be modified such as by amino acid replacement, deletion or substitution. The identified residues can be replaced or substituted with any another amino acid. Alternatively, amino acid insertions can be used to alter the conformation of a targeted amino acid residue or the protein structure in the vicinity of a targeted amino acid residue.

Any amino acid residue can be substituted for the endogenous amino acid residue at the identified positions. Typically, the replacement amino acid is chosen such that it interferes with the interaction between FIX and AT-III or heparin. For example, modifications can be made at amino acid positions 260, 293, 333, 338, 346, 400 and 410 (corresponding to amino acid positions 95, 126, 165, 170, 178, 230, 233 and 240, respectively, by chymotrypsin numbering) to interfere with the interaction of the FIX polypeptide with heparin. In other examples, modifications are made at amino acid positions 203, 204, 205, 228, 239, 312, 314, 316, 318, 319, 321, and 342 (corresponding to amino acid positions 39, 40, 41, 63, 74, 143, 145, 148, 150, 151, 153, and 174, respectively, by chymotrypsin numbering) to interfere with the interaction of the FIX polypeptide with AT-III.

In some examples, a new glycosylation site is introduced by amino acid replacement. The carbohydrate moiety that is linked to the new glycosylation site can sterically hinder the interaction of the modified FIX with the AT-III/heparin complex, resulting in increased resistance of the modified FIX polypeptide to the inhibitory effects of AT-III/heparin. For example, the glutamic acid (Glu, E) at position 410 (corresponding to position 240 by chymotrypsin numbering) can be replaced with an asparagine (Asn, N) to introduce a new glycosylation site at position 410. In other examples, the glutamic acid (Glu, E) at position 239 (corresponding to position 74 by chymotrypsin numbering) is replaced with an asparagine (Asn, N) to introduce a new glycosylation site at position 239. Other mutations that introduce a new glycosylation site to increase resistance to AT-III/heparin include, for example, D203N/F205T, R318N/A320S, N260S, and F314N/K316S (corresponding to D39N/F41T, R150N/A152S, N95S, and F145N/K148S, by chymotrypsin numbering).

In other examples in which modifications are made to increase resistance to AT-III, AT-III/heparin and/or heparin, the valine residue at position 202 (corresponding to position 38 by chymotrypsin numbering) is replaced with a methionine (Met, M) or tyrosine (Tyr, Y); the aspartic acid (Asp, D) at position 203 (corresponding to position 39 by chymotrypsin numbering) is replaced with a methionine (Met, M) or tyrosine (Tyr, Y); the alanine (Ala, A) at position 204 (corresponding to position 40 by chymotrypsin numbering) is replaced with a methionine (Met, M) or tyrosine (Tyr, Y); the glutamic acid at position 239 (corresponding to position 74 by chymotrypsin numbering) is replaced with serine (Ser, S), alanine (Ala, A), arginine (Arg, R), or lysine (Lys, K); the histidine at position 257 (corresponding to position 92 by chymotrypsin numbering) is replaced with phenylalanine (Phe, F), tyrosine (Tyr, Y), glutamic acid (Glu, E) or serine (Ser, S); the lysine (Lys, K) at position 293 (corresponding to position 143 by chymotrypsin numbering) is replaced with alanine (Ala, A) or glutamine (Gln, Q); the arginine (Arg, R) at position 312 (corresponding to position 143 by chymotrypsin numbering) is replaced with alanine (Ala, A) or glutamine (Gln, Q); the lysine at position 316 (corresponding to 148 by chymotrypsin numbering) is replaced with asparagine (Asn, N), alanine (Ala, A), glutamic acid (Glu, E), serine (Ser, S) or methionine (Met, M); the arginine (Arg, R) at position 318 (corresponding to position 150 by chymotrypsin numbering) is replaced with alanine (Ala, A), glutamic acid (Glu, E) tyrosine (Tyr, Y), phenylalanine (Phe, F) or tryptophan (Trp, W); the arginine (Arg, R) at position 333 (corresponding to position 165 by chymotrypsin numbering) is replaced with alanine (Ala, A) or glutamic acid (Glu, E); the arginine (Arg, R) at position 338 (corresponding to position 170 by chymotrypsin numbering) is replaced with alanine (Ala, A) or glutamic acid (Glu, E); the lysine (Lys, K) at position 400 (corresponding to position 230 by chymotrypsin numbering) is replaced with alanine (Ala, A) or glutamic acid (Glu, E); and/or the arginine (Arg, R) at position 403 (corresponding to position 233 by chymotrypsin numbering) is replaced with alanine (Ala, A), glutamic acid (Glu, E) or aspartic acid (Asp, D).

Provided herein are modified FIX polypeptides that contains an amino acid replacement at residue R318 or at a residue in a FIX polypeptide corresponding to 318 that is a tyrosine, e.g., R318Y, or is a conservative amino acid replacement thereof. For example, conservative amino acid residues for tyrosine include, but are not limited to, phenylalanine (F) or tryptophan (W). Also provided are modified FIX polypeptides that contains an amino acid replacement at residue R403 or at a residue in a FIX polypeptide corresponding to 403 that is a glutamic acid, e.g., R403E, or is a conservative amino acid replacement thereof. For example, conservative amino acid residues for glutamic acid include, but are not limited to, aspartic acid (D).

In a further embodiment, combination mutants can be generated. Included among such combination mutants are those having two or more mutations at amino acid positions 202, 203, 204, 257, 239, 293, 312, 316, 318, 333, 338, 400, 403, and 410 (corresponding to amino acid positions 38, 39, 40, 74, 92, 126, 143, 148, 150, 165, 170, 230, 233, and 240, respectively, by chymotrypsin numbering). For example, a modified FIX polypeptide can possess amino acid substitutions at 2, 3, 4, 5 or more of the identified positions. Hence, a modified polypeptide can display 1, 2, 3, 4, 5 or more mutations that can result in increased resistance of the modified FIX polypeptide to the inhibitory effects of AT-III, AT-III/heparin and/or heparin. Any one or more of the mutations described herein to increase resistance of the modified FIX polypeptide to the inhibitory effects of AT-III, AT-III/heparin and/or heparin can be combined. Table 8 provides non-limiting examples of exemplary amino acid replacements at the identified residues, corresponding to amino acid positions of a mature FIX polypeptide as set forth in SEQ ID NO:3. Included amongst these are exemplary combination mutations. As noted, such FIX polypeptides are designed to increase resistance to AT-III, AT-III/heparin and/or heparin, and therefore have increased coagulant activity in vivo, ex vivo, or in in vitro assays that include ATIII, heparin/ATIII, heparin, plasma, serum, or blood. In reference to such mutations, the first amino acid (one-letter abbreviation) corresponds to the amino acid that is replaced, the number corresponds to the position in the mature FIX polypeptide sequence with reference to SEQ ID NO:3, and the second amino acid (one-letter abbreviation) corresponds to the amino acid selected that replaces the first amino acid at that position. The amino acid positions for mutation also are referred to by the chymotrypsin numbering scheme. In Table 8 below, the sequence identifier (SEQ ID NO.) is identified in which exemplary amino acid sequences of the modified FIX polypeptide are set forth.

TABLE 8

| Mutation | Mutation | SEQ |
|---|---|---|
| (Mature FIX | (Chymotrypsin | ID |
| Numbering) | Numbering) | NO. |
| R318A | R150A | 120 |
| R318E | R150E | 121 |
| R318Y | R150Y | 122 |
| R318F | R150F | 413 |
| R318W | R150W | 414 |
| R312Q | R143Q | 123 |
| R312A | R143A | 124 |
| R312Y | R143Y | 125 |
| R312L | R143L | 126 |
| V202M | V38M | 127 |
| V202Y | V38Y | 128 |
| D203M | D39M | 129 |
| D203Y | D39Y | 130 |
| A204M | A40M | 131 |
| A204Y | A40Y | 132 |
| K400A/R403A | K230A/R233A | 133 |
| K400E/R403E | K230E/R233E | 134 |
| R403A | R233A | 135 |
| R403E | R233E | 136 |
| R403D | R233D | 417 |
| K400A | K230A | 137 |
| K400E | K230E | 138 |
| K293E | K126E | 139 |
| K293A | K126A | 140 |
| R333A | R165A | 141 |
| R333E | R165E | 142 |
| R333S | R165S | 186 |
| R338A | R170A | 143 |
| R338E | R170E | 144 |
| R338L | R170L | 187 |
| R338A/R403A | R170A/R233A | 145 |
| R338E/R403E | R170E/R233E | 146 |
| K293A/R403A | K126A/R233A | 147 |
| K293E/R403E | K126E/R233E | 148 |
| K293A/R338A/R403A | K126A/R170A/R233A | 149 |
| K293E/R338E/R403E | K126E/R170E/R233E | 150 |
| R318A/R403A | R150A/R233A | 151 |
| R318E/R403E | R150E/R233E | 152 |
| R318Y/R338E/R403E | R150Y/R170E/R233E | 156 |
| R318Y/R338E | R150Y/R170E | 188 |
| R318N/A320S | R150N/A152S | 119 |
| K316N | K148N | 189 |
| K316A | K148A | 190 |
| K316E | K148E | 191 |
| K316S | K148S | 192 |
| K316M | K148M | 193 |
| E239N | E74N | 109 |
| E239S | E74S | 194 |
| E239A | E74A | 195 |
| E239R | E74R | 196 |
| E239K | E74K | 197 |
| H257F | H92F | 198 |
| H257Y | H92Y | 199 |
| H257E | H92E | 200 |
| H257S | H92S | 201 |
| E410N | E240N | 108 |
| N260S | N95S | 116 |
| F314N/K316S | F145N/K148S | 113 |

The modifications described herein to increase resistance to an inhibitor, such as AT-III and/or heparin, can be combined with any other mutation described herein or known in the art. Typically, the resulting modified FIX polypeptide exhibits increased coagulant activity compared to an unmodified FIX polypeptide. For example, one or more modifications that increase resistance to an inhibitor, such as AT-III and/or heparin, can be combined with modification(s) that introduce a non-native glycosylation site, eliminate one or more native glycosylation sites, eliminate one or more of the native sulfation, phosphorylation or hydroxylation sites, increase catalytic activity, increase intrinsic activity, increase binding to phospholipids, or improve pharmacokinetic and/or pharmacodynamic properties. The resulting modified FIX polypeptide typically exhibits increased coagulant activity compared to an unmodified FIX polypeptide.

Modified FIX polypeptides that have increased resistance for AT-III alone, the AT-III/heparin complex and/or heparin alone, can exhibit a reduction in the affinity for heparin, the extent of inhibition under specified conditions, or in the second order rate constant for inhibition by ATIII or heparin/ATIII at least or at least about 1%, 2%, 3%, 4%, 5%, 6%, 7%, 8%, 9%, 10%, 20%, 30%, 40%, 50%, 60%, 70%, 80%, 90%, 95%, 99% or more compared to the affinity, extent of inhibition, or the second order rate constant for inhibition of unmodified or wild-type FIX polypeptide either in vivo or in vitro. Thus, the modified FIX polypeptides can exhibit increased resistance to AT-III alone, the AT-III/heparin complex and/or heparin alone that is at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more of the resistance exhibited by an unmodified FIX polypeptide. Increased resistance to AT-III, the AT-III/heparin complex and/or heparin by such modified FIX polypeptides also can be manifested as increased coagulation activity or improved duration of coagulation activity in vivo or in vitro in the presence of AT-III, the AT-III/heparin complex, heparin, blood, plasma, or serum. The coagulation activity of the modified FIX polypeptides can be increased by at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro. Modified FIX polypeptides containing modifications that increase resistance to AT-III, the heparin/AT-III complex, and/or heparin also can exhibit an enhanced therapeutic index compared with unmodified FIXa.

c. Mutations to Increase Catalytic Activity

The modified FIX polypeptides provided herein can contain one or more modifications to increase the catalytic activity of the polypeptide compared to an unmodified FIX. For example, modifications can be made to the amino acids that are involved in the interaction of FIX with its cofactor, FVIIIa, such that the resulting modified FIX polypeptide has increased affinity for FVIIIa, and thereby displays increased activity toward FX under conditions in which FVIIIa is not present at saturating concentrations. Modifications also can be made to the protease domain of the FIX polypeptide, such that the activity or catalytic efficiency of the modified FIX polypeptide for activation of FX, in the presence and/or absence of the co-factor FVIIIa, is increased compared to the activity or catalytic efficiency of the unmodified polypeptide.

Exemplary modifications that can be included in the modified FIX polypeptides provided herein include amino acid replacements at positions 259, 265, 345, 410, and 412 (corresponding to 94, 98, 177, 240, and 242, by chymotrypsin numbering). The amino acids at these positions can be replaced by any other amino acid residue. In some examples, the tyrosine at position 259 is replaced with a phenylalanine; the lysine at position 265 is replaced with a threonine; and/or the tyrosine at position 345 is replaced with a threonine. In further example, the glutamic acid at position 410 is replaced with a glutamine, serine, alanine or aspartic acid. In one example, the threonine at position 412 is replaced with a valine or an alanine.

The above mentioned modifications are exemplary only. Many other modifications described herein also result in increased catalytic activity. For example, modifications that are introduced into the FIX polypeptide to increase resistance to an inhibitor, such as AT-III and/or heparin, introduce a non-native glycosylation site, eliminate one or more native glycosylation sites, eliminate one or more of the native sulfation, phosphorylation or hydroxylation sites, increase intrinsic activity, increase binding to phospholipids, decrease binding to LRP, and/or improve pharmacokinetic and/or pharmacodynamic properties, can also result in a modified FIX polypeptide that exhibits increased activity.

Table 9 provides non-limiting examples of exemplary amino acid replacements at the identified residues, corresponding to amino acid positions of a mature FIX polypeptide as set forth in SEQ ID NO:3. In reference to such mutations, the first amino acid (one-letter abbreviation) corresponds to the amino acid that is replaced, the number corresponds to the position in the mature FIX polypeptide sequence with reference to SEQ ID NO:3, and the second amino acid (one-letter abbreviation) corresponds to the amino acid selected that replaces the first amino acid at that position. The amino acid positions for mutation also are referred to by the chymotrypsin numbering scheme. In Table 9 below, the sequence identifier (SEQ ID NO) is identified in which exemplary amino acid sequences of the modified FIX polypeptide are set forth.

TABLE 9

| Mutation | Mutation | SEQ |
|---|---|---|
| (Mature FIX | (Chymotrypsin | ID |
| Numbering) | Numbering) | NO |
| T412A | T242A | 202 |
| T412V | T242V | 203 |
| E410Q | E240Q | 174 |
| E410S | E240S | 175 |
| E410A | E240A | 176 |
| E410D | E240D | 206 |
| Y259F/K265T/Y345T | Y94F/K98T/Y177T | 216 |

The modifications described herein to increase catalytic activity can be combined with any other mutation described herein or known in the art. Typically, the resulting modified FIX polypeptide exhibits increased coagulant activity compared to an unmodified FIX polypeptide. For example, one or more modifications that increase catalytic activity can be combined with modification(s) that increase resistance to an inhibitor, such as AT-III and/or heparin, introduce a non-native glycosylation site, eliminate one or more native glycosylation sites, eliminate one or more of the native sulfation, phosphorylation or hydroxylation sites, increase intrinsic activity, increase binding to phospholipids, or improve pharmacokinetic and/or pharmacodynamic properties. The resulting modified FIX polypeptide typically exhibits increased coagulant activity compared to an unmodified FIX polypeptide.

Modified FIX polypeptides that have increased catalytic activity can exhibit at least or about 1%, 2%, 3%, 4%, 5%, 6%, 7%, 8%, 9%, 10%, 20%, 30%, 40%, 50%, 60%, 70%, 80%, 90%, 95%, 99% or more activity compared to the catalytic activity of unmodified or wild-type FIX polypeptide either in vivo or in vitro. Increased catalytic activity of such modified FIX polypeptides also can be manifested as increased coagulation activity, duration of coagulation activity and/or enhanced therapeutic index. The coagulation activity of the modified FIX polypeptides can be increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro.

d. Mutations to Decrease LRP Binding

FIXa can be cleared from systemic circulation by binding the low-density lipoprotein receptor-related protein (LRP), which is a membrane glycoprotein that is expressed on a variety of tissues, including liver, brain, placenta and lung. Thus, provided herein are modified FIX polypeptides that exhibit decreased binding to the LRP. This can result in improved pharmacokinetic properties of the modified FIX polypeptide, including, for example, i) decreased clearance, ii) altered volume of distribution, iii) enhanced in vivo recovery, iv) enhanced total protein exposure in vivo (i.e., AUC), v) increased serum half-life (α, β, and/or γ phase), and/or vi) increased mean resonance time (MRT). Such modified FIX polypeptides can exhibit increased coagulant activity.

The modified FIX polypeptide provided herein can contain one or more modifications in the LRP-binding site. This binding site is postulated to be located in a loop in the protease domain spanning residues 342 to 346 of the mature FIX polypeptide set forth in SEQ ID NO:3. Modification of one or more of the residues at positions 342-346 (corresponding to positions 174-178 by chymotrypsin numbering), such as by amino acid replacement, insertion or deletion, can interfere with the interaction between the modified FIX polypeptide and LRP, resulting in decreased binding affinity. The binding of the modified FIX polypeptides to LRP can be tested using assays known to one of skill in the art (see, e.g., Rohlena et al., (2003) J. Biol. Chem. 278:9394-9401). The resulting improved pharmacokinetic properties also can be tested using well known in vivo assays, including those described below.

Exemplary modifications that can be included in the modified FIX polypeptides provided herein include amino acid replacements at positions 343, 344, 345, and 346 (corresponding to 175, 176, 177, and 178, by chymotrypsin numbering). The amino acids at these positions can be replaced by any other amino acid residue. In some examples, the threonine at position 343 is replaced with a glutamine, glutamic acid, aspartic acid or arginine; the phenylalanine at position 344 is replaced with an isoleucine; the tyrosine at position 345 is replaced with a threonine, alanine or an alanine; and/or the asparagine at position 346 is replaced with an aspartic acid or a tyrosine. Any one or more of these exemplary amino acid replacements can be combined with each other or with other modifications described herein.

Provided herein are modified FIX polypeptides that contains an amino acid replacement at residue T343 or at a residue in a FIX polypeptide corresponding to 343 that is an arginine, e.g., T343R, or is a conservative amino acid replacement thereof. For example, conservative amino acid residues for arginine include, but are not limited to, lysine (K).

Table 10 provides non-limiting examples of exemplary amino acid replacements at the identified residues, corresponding to amino acid positions of a mature FIX polypeptide as set forth in SEQ ID NO:3. In reference to such mutations, the first amino acid (one-letter abbreviation) corresponds to the amino acid that is replaced, the number corresponds to the position in the mature FIX polypeptide sequence with reference to SEQ ID NO:3, and the second amino acid (one-letter abbreviation) corresponds to the amino acid selected that replaces the first amino acid at that position. The amino acid positions for mutation also are referred to by the chymotrypsin numbering scheme. In Table 10 below, the sequence identifier (SEQ ID NO) is identified in which exemplary amino acid sequences of the modified FIX polypeptide are set forth.

TABLE 10

| Mutation | Mutation | SEQ |
|---|---|---|
| (Mature FIX | (Chymotrypsin | ID |
| Numbering) | Numbering) | NO. |
| N346D | N178D | 207 |
| N346Y | N178Y | 208 |
| T343R | T175R | 209 |
| T343E | T175E | 210 |
| T343D | T175D | 416 |
| T343Q | T175Q | 211 |
| F342I | F1741 | 212 |
| Y345A | Y177A | 213 |
| Y345T | Y177T | 214 |
| T343R/Y345T | T175R/Y177T | 215 |
| T343R/N346D | T175R/N178D | 409 |
| T343R/N346Y | T175R/N178Y | 410 |

The modifications described herein to decrease binding to LRP can be combined with any other mutation described herein or known in the art. Typically, the resulting modified FIX polypeptide exhibits increased coagulant activity compared to an unmodified FIX polypeptide. For example, one or more modifications that decrease binding to LRP can be combined with modification(s) that increase resistance to an inhibitor, such as AT-III and/or heparin, increase catalytic activity, introduce a non-native glycosylation site, eliminate one or more native glycosylation sites, eliminate one or more of the native sulfation, phosphorylation or hydroxylation sites, increase activity in the presence and/or absence of FVIIIa, increase binding to phospholipids, or improve pharmacokinetic and/or pharmacodynamic properties. The resulting modified FIX polypeptide typically exhibits increased coagulant activity compared to an unmodified FIX polypeptide.

Modified FIX polypeptides that have decreased binding to LRP can exhibit at a decrease of at least or about 1%, 2%, 3%, 4%, 5%, 6%, 7%, 8%, 9%, 10%, 20%, 30%, 40%, 50%, 60%, 70%, 80%, 90%, 95%, 99% or more compared to the binding of unmodified or wild-type FIX polypeptide to LRP in vitro. Decreased binding to LRP by such modified FIX polypeptides can result in improved pharmacokinetic properties, such as i) decreased clearance, ii) altered volume of distribution, iii) enhanced in vivo recovery, iv) enhanced total protein exposure in vivo (i.e., AUC), v) increased serum half-life (αγ, β, and/or γ phase), and/or vi) increased mean resonance time (MRT). Further, such alterations can result in increased coagulant activity, duration of coagulation activity and/or enhanced therapeutic index. The coagulation activity of the modified FIX polypeptides can be increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro.

e. Other Mutations to Alter Post-Translational Modifications

Wild-type FIX is post-translationally modified upon expression in mammalian cells. The Factor IX precursor polypeptide undergoes extensive posttranslational modification to become the mature zymogen that is secreted into the blood. Such posttranslational modifications include γ-carboxylation, β-hydroxylation, O- and N-linked glycosylation, sulfation and phosphorylation. As discussed above, the levels of glycosylation can be altered by, for example, introducing new non-native glycosylation sites and/or eliminating native glycosylation sites. Similarly, other posttranslational modifications can be altered, such as by introducing and/or eliminating γ-carboxylation, β-hydroxylation, sulfation and/or phosphorylation sites.

Any one or more of the native γ-carboxylation, β-hydroxylation, sulfation or phosphorylation sites can be eliminated, such as by amino acid replacement or deletion. For example, unmodified FIX polypeptides can be modified by amino acid replacement of any one or more of the twelve glutamic acid residues (corresponding to positions 7, 8, 15, 17, 20, 21, 26, 27, 30, 33, 36, and 40 of the mature FIX polypeptide set forth in SEQ ID NO:3) in the Gla domain. These residues typically are γ-carboxylated to γ-carboxyglutamyl (or Gla) in wild-type FIX. Thus, removal of the glutamic acid residues, such as by amino acid substitution or deletion, can reduce the level of γ-carboxylation in a modified FIX polypeptide compared to the unmodified FIX polypeptide. Similarly, the aspartic acid residue at position 64, which normally is β-hydroxylated in wild-type FIX, can be removed, such as by amino acid substitution or deletion. Additional post-translational modification sites that can be eliminated include, for example, the tyrosine at position 155, which typically is sulfated in wild-type FIX, and the serine residue at position 158, which typically is phosphorylated in wild-type FIX.

In other examples, non-native post-translational modification sites can be introduced, such as by amino acid replacement or insertion. For example, additional glutamic acid residues can be introduced into the Gla domain. Such glutamic acid residues could be γ-carboxylated to γ-carboxyglutamyl (or Gla) in the modified FIX polypeptide upon expression in, for example, a mammalian cell. Similarly, one or more non-native β-hydroxylation, sulfation or phosphorylation sites can be introduced.

Provided herein are modified FIX polypeptides that have one or more of the native posttranslational modification sites eliminated. The modified FIX polypeptides that have been modified to eliminate one or more post-translational modification sites, including γ-carboxylation, β-hydroxylation, sulfation and/or phosphorylation sites, retain at least one activity of the unmodified FIX polypeptide. In some examples, the modified FIX polypeptide retains at least or at least about 5%, 10%, 20%, 30%, 40%, 50%, 60%, 70%, 80%, 90%, 95% or more of the catalytic activity of the unmodified FIX polypeptide. In other examples, the modified FIX polypeptide retains at least or at least about 5%, 10%, 20%, 30%, 40%, 50%, 60%, 70%, 80%, 90%, 95% or more of the binding activity for FVIIIa of the unmodified FIX polypeptide. In some assays and/or under some conditions, the modified FIX polypeptides can exhibit increased activity compared with the unmodified FIX protein (e.g., increased pharmacodynamic activity in vivo, and/or activity in the presence of AT-III/heparin or plasma).

Provided herein are modified FIX polypeptides that contains an amino acid replacement at residue Y155 or at a residue in a FIX polypeptide corresponding to 155 that is a phenylalanine, e.g., Y155F, or is a conservative amino acid replacement thereof. For example, conservative amino acid residues for phenylalanine include, but are not limited to, methionine (M), leucine (L) or tyrosine (Y).

Table 11 provides non-limiting examples of exemplary amino acid replacements, corresponding to amino acid positions of a mature FIX polypeptide as set forth in SEQ ID NO:3, that are included in a modified FIX polypeptide to eliminate a native β-hydroxylation, sulfation and/or phosphorylation sites at positions 64, 155 and 158, respectively. In Table 11 below, the sequence identifier (SEQ ID NO) is identified in which exemplary amino acid sequences of the modified FIX polypeptide are set forth.

TABLE 11

| Mutation | Mutation | SEQ |
|---|---|---|
| (Mature FIX | (Chymotrypsin | ID |
| Numbering) | Numbering) | NO. |
| D64N | D[64]N | 83 |
| D64A | D[64]A | 84 |
| Y155F | Y[155]F | 76 |
| Y155H | Y[155]H | 93 |
| Y155Q | Y[155]Q | 94 |
| T155L | Y[155]L | 415 |
| S158A | S[158]A | 95 |
| S158D | S[158]D | 96 |
| S158E | S[158]E | 97 |

The modifications described herein to eliminate β-hydroxylation, sulfation and/or phosphorylation sites can be combined with any other mutation described herein or known in the art. Typically, the resulting modified FIX polypeptide exhibits increased coagulant activity compared to an unmodified FIX polypeptide. For example, one or more modifications that eliminate one or more native β-hydroxylation, sulfation and/or phosphorylation sites can be combined with modification(s) that increase resistance to an inhibitor, such as AT-III and/or heparin, alter glycosylation, such as increase glycosylation, increase catalytic activity, increase intrinsic activity, increase binding to phospholipids, or improve pharmacokinetic and/or pharmacodynamic properties.

The modified FIX polypeptides provided herein that eliminate one or more native β-hydroxylation, sulfation and/or phosphorylation sites retain at least one activity of FIX, such as, for example, catalytic activity for its substrate, FX, or binding to the co-factor, FVIIIa. Typically, the modified FIX polypeptides provided herein retain at least or at least about 5%, 10%, 20%, 30%, 40%, 50%, 60%, 70%, 80%, 90%, 95% or more of the catalytic activity exhibited by an unmodified FIX polypeptide. In some instances, the coagulant activity of the modified FIX polypeptides is increased by at least or at least about 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, or more compared to the coagulation activity of unmodified or wild-type FIX polypeptide either in vivo or in vitro.

2. Combination Modifications

The modified FIX polypeptides provided herein that contain one or more non-native glycosylation sites, have one or more native glycosylation sites eliminated, have one or more native β-hydroxylation, sulfation and/or phosphorylation sites eliminated, or that have modifications that can result in increased resistance to inhibitors, such as AT-III, AT-III/heparin and/or heparin, compared to a wild-type FIX polypeptide, also can contain other modifications. In some examples, the modified FIX polypeptides contain modifications that introduce one or more non-native glycosylation sites and also contain modifications that interfere with the interaction between FIX and inhibitors, such as AT-III, the AT-III/heparin complex and/or and heparin. In other examples, modifications that eliminate one or more native β-hydroxylation, sulfation and/or phosphorylation sites can be combined with modifications that increase resistance to inhibitors, and/or modifications that introduce one or more glycosylation sites. Thus, one or more of the mutations set forth in Tables 3-9 above, can be combined with any of the other mutations set forth in Tables 3-9 above. Thus, included among the modified FIX polypeptides provided herein are those that exhibit increased glycosylation, such as N-glycosylation; increased resistance to AT-III, AT-III/heparin, and/or heparin; decreased β-hydroxylation, sulfation and/or phosphorylation; and/or increased catalytic activity compared with an unmodified FIX polypeptide.

Further, any of the modified FIX polypeptides provided herein can contain any one or more additional modifications. In some examples, the additional modifications result in altered properties and/or activities compared to an unmodified FIX polypeptide. Typically, such additional modifications are those that themselves result in an increased coagulant activity of the modified polypeptide and/or increased stability of the polypeptide. Accordingly, the resulting modified FIX polypeptides typically exhibit increased coagulant activity.

The additional modifications can include, for example, any amino acid substitution, deletion or insertion known in the art, typically any that increases the coagulant activity and/or stability of the FIX polypeptide. Any modified FIX polypeptide provided herein can contain 1, 2, 3, 4, 5, 6, 7, 8, 9, 10, 11, 12, 13, 14, 15, 16, 17, 18, 19, 20, or more, additional amino acid modifications. Typically, the resulting modified FIX polypeptide retains at least one activity of the wild-type or unmodified polypeptide, such as, for example, catalytic activity, or binding to the co-factor, FVIIIa.

Additional modifications in the primary sequence can be made to the FIX polypeptide to effect post-translational modifications. For example, the modified FIX polypeptides provided herein can contain non-native glycosylation sites including and other than those described above, such as any of those described in the art, including non-native O-linked or S-linked glycosylation sites described in U.S. Patent Publication No. 2008/0280818, or the non-native glycosylation sites described in International Application Publication Nos. WO 2009/1300198 and WO 2009/137254.

In other examples, the additional modification can be made to the FIX polypeptide sequence such that its interaction with other factors, molecules and proteins is altered. For example, the amino acid residues that are involved in the interaction with Factor X can be modified such that the affinity and/or binding of the modified FIX polypeptide to FX is increased. Other modifications include, but are not limited to, modification of amino acids that are involved in interactions with FVIIIa, heparin, antithrombin III and phospholipids.

Additional modifications also can be made to a modified FIX polypeptide provided herein that alter the conformation or folding of the polypeptide. These include, for example, the replacement of one or more amino acids with a cysteine such that a new disulfide bond is formed, or modifications that stabilize an α-helix conformation, thereby imparting increased activity to the modified FIX polypeptide.

Modifications also can be made to introduce amino acid residues that can be subsequently linked to a moiety, such as one that acts to increase stability of the modified FIX polypeptide. For example, cysteine residues can be introduced to facilitate conjugation to a polymer, such polyethylene glycol (PEG) (International App. Pub. No. WO 2009/140015). The stability of a FIX polypeptide also can be altered by modifying potential proteolytic sites, such as removing potential proteolytic sites, thereby increasing the resistance of the modified FIX polypeptide to proteases (see, e.g., U.S. Pat. Pub. No. 2008/0102115).

Additionally, amino acids substitutions, deletions or insertions can be made in the endogenous Gla domain such that the modified FIX polypeptide displays increased binding and/or affinity for phospholipid membranes. Such modifications can include single amino acid substitution, deletions and/or insertions, or can include amino acid substitution, deletion or insertion of multiple amino acids. For example, all or part of the endogenous Gla domain can be replaced with all or part of a heterologous Gla domain. In other examples, the modified FIX polypeptides provided herein can display deletions in the endogenous Gla domain, or substitutions in the positions that are normally gamma-carboxylated. Alternatively, amino acid substitutions can be made to introduce additional, potential gamma-carboxylation sites.

The following sections describe non-limiting examples of exemplary modifications described in the art to effect increased stability and/or coagulant activity of a FIX polypeptide. As discussed above, such modifications also can be additionally included in any modified FIX polypeptide provided herein. The amino acid positions referenced below correspond to the mature FIX polypeptide as set forth in SEQ ID NO:3. Corresponding mutations can be made in other FIX polypeptides, such as allelic, species or splice variants of the mature FIX polypeptide set forth in SEQ ID NO:3.

a. Modifications to Increase Activity

In one example, additional modifications can be made to a modified factor IX polypeptide provided herein that result in increased catalytic activity toward factor X. For example, modifications can be made to the amino acids that are involved in the interaction with its cofactor, FVIIIa, such that the resulting modified FIX polypeptide has increased affinity for FVIIIa, and thereby displays increased activity toward FX under conditions in which FVIIIa is not saturating. Modifications can also be made in FIX that increase the catalytic efficiency of FIXa polypeptides and/or the FIXa/FVIIIa complex, compared to the activity of the unmodified FIXa polypeptide or FIXa/FVIIIa complex, for activation of the substrate FX.

Examples of additional modifications that can be included in the modified FIX polypeptides described herein to increase the intrinsic activity of the modified FIX polypeptide include, but are not limited to, those described in Hopfner et al., (1997) EMBO J. 16:6626-6635; Kolkman et al., (2000) Biochem. 39:7398-7405; Sichler et al., (2003) J. Biol. Chem. 278:4121-4126; Begbie et al., (2005) J. Thromb. Haemost. 94(6):1138-47; U.S. Pat. No. 6,531,298; and U.S. Patent Publication Nos. 2008/0167219 and 2008/0214461. Non-limiting examples of exemplary amino acid modifications described in the art that can result in increased intrinsic activity of the modified FIX polypeptide include any one or more of V86A, V86N, V86D, V86E, V86Q, V86G, V86H, V86I, V86L, V86M, V86F, V86S, V86T, V86W, V86Y, Y259F, A261K, K265T, E277V, E277A, E277N, E277D, E277Q, E277G, E277H, E277I, E277L, E277M, E277F, E277S, E277T, E277W, E277Y, R338A, R338V, R338I, R338F, R338W, R338S, R338T, Y345F, I383V, and E388G. For example, a modified FIX polypeptide provided herein can contain the amino acid substitutions Y259F/K265T, Y259F/K265T/Y345F, Y259F/A261K/K265T/Y345F, Y259F/K265T/Y345F/I383V/E388G, or Y259F/A261K/K265T/Y345F/I383V/E388G. In another example, the modified FIX polypeptides provided herein can contain modifications that remove the activation peptide (Δ155-177) (see, e.g., Begbie et al., (2005) J. Thromb. Haemost. 94(6):1138-1147), which can both increase activity and decrease clearance in vivo.

b. Modifications that Increase Affinity for Phospholipids or Reduce Binding to Collagen

The modified FIX polypeptides provided herein also can contain one or more additional modifications to increase affinity for phospholipids. The coagulant activity of FIX can be enhanced by increasing the binding and/or affinity of the polypeptide for phospholipids, such as those expressed on the surface of activated platelets. This can be achieved, for example, by modifying the endogenous FIX Gla domain. Modification can be effected by amino acid substitution at one or more positions in the Gla domain of a FIX polypeptide that result in a modified FIX polypeptide with increased ability to bind phosphatidylserine and other negatively charged phospholipids. Examples of additional modifications to increase phospholipid binding and/or affinity and that can be made to a modified FIX polypeptide provided herein, include, but are not limited to, those described in U.S. Pat. No. 6,017,882. For example, a modified FIX polypeptide provided herein can contain one or more modifications at amino acid positions 11, 12, 29, 33 and/or 34 (corresponding to a mature FIX polypeptide set forth in SEQ ID NO:3). Exemplary of such modifications are amino acid substitutions K5I, K5L, K5F, K5E, Q11E, Q11D, R16E, R29F and/or N34E, N34D, N34F, N34I, N34L, T35D, and T35E.

In another aspect, the modified FIX polypeptides provided herein also can contain one or more additional modifications to reduce affinity for collagen. The coagulant activity of FIX can be enhanced by reducing the binding and/or affinity of the polypeptide for collagen IV, which is present on the surface of the extracellular matrix on endothelial cells. A reduced binding to collagen IV can result in increased circulation of the modified FIX polypeptides and, thus, increased coagulant activity in vivo. This can be achieved, for example, by modifying the FIX Gla domain at amino acid residues 3 to 11 of a mature FIX polypeptide set forth in SEQ ID NO:3, which are responsible for the interaction with collagen IV (see, e.g., Cheung et al., (1992) J. Biol. Chem. 267:20529-20531; Cheung et al., (1996) Proc. Natl. Acad. Sci. U.S.A. 93:11068-11073). Modification can be effected by amino acid substitution at one or more positions in the Gla domain of a FIX polypeptide that result in a modified FIX polypeptide with decreased ability to bind collagen IV. Examples of additional modifications to increase phospholipid binding and/or affinity and that can be made to a modified FIX polypeptide provided herein, include, but are not limited to, those described in Schuettrumpf et al., (2005) Blood 105(6):2316-23; Melton et al., (2001) Blood Coagul. Fibrinolysis 12(4):237-43; and Cheung et al., (1996) Proc. Natl. Acad. Sci. U.S.A. 93:11068-11073. For example, a modified FIX polypeptide provided herein can contain are amino acid substitutions K5A and/or V10K.

c. Additional Modifications to Increase Resistance to Inhibitors

Additional modifications can be included that increase the activity of the FIX polypeptide by increasing the resistance of the modified FIX polypeptide to inhibitors, such as, for example, inhibition by antithrombin III (AT-III)/heparin. Typically, this can be achieved by modifying one or more residues that are involved in the interaction with AT-III, heparin or the AT-III/heparin complex. Exemplary of such modifications include those described, for example, in U.S. Pat. No. 7,125,841; U.S. Pat. Pub. No. 2004/0110675; Int. App. Pub. No. WO 2002/040544; Chang, J. et al., (1998) J. Biol. Chem. 273(20):12089-94; Yang, L. et al., (2002) J. Biol. Chem. 277(52):50756-60; Yang, L. et al., (2003) J. Biol. Chem. 278(27):25032-8; Rohlena et al., (2003) J. Biol. Chem. 278(11):9394-401; Sheehan et al., (2006) Blood 107(10):3876-82; and Buyue et al. (2008) Blood 112:3234-3241. Non-limiting examples of modifications that can be included to decrease inhibition by AT-III and/or heparin, include, but are not limited to, modifications at amino acid positions corresponding to amino acid positions R252, H256, H257, K265, H268, K293, R318, R333, R338, K400, R403, K409, or K411, of a mature FIX polypeptide set forth in SEQ ID NO:3. For example, the FIX polypeptides provided herein can contain the amino acid substitutions R252A, H257A, H268A, K293A, R318A, R333A, R338A, K400A, R403A, R403E, and/or K411A.

d. Additional Modifications to Alter Glycosylation

Modifications, in addition to those described above can be incorporated into the modified FIX polypeptides provided herein to alter the glycosylation of the modified FIX polypeptides compared to an unmodified FIX polypeptide. For example, the modified FIX polypeptides can contain one or more modifications that introduce one or more non-native glycosylation sites into the modified FIX polypeptide. Thus, when expressed in an appropriate system, the modified FIX polypeptides can exhibit altered glycosylation patterns compared to an unmodified FIX polypeptide. In some examples, the modified FIX polypeptides exhibit increased glycosylation compared to an unmodified FIX polypeptide, such as increased N-glycosylation or increased O-glycosylation.

Examples of additional modifications that can be included in the modified FIX polypeptides provided herein to alter the glycosylation profile of a FIX polypeptide include, but are not limited to, those described in International Application Publication Nos. WO 2009/130198, WO 2009/051717 and WO 2009/137254. Exemplary modifications that can be included in a modified FIX polypeptide provided herein to increase glycosylation include, but are not limited to, Y1N, Y1N+S3T, S3N+K5S/T, G4T, G4N+L6S/T, K5N+E7T, L6N+EBT, E7N+F9T, F9N+Q11S/T, V10N+G12S/T, Q11N+N13T, G12N+L14S/T, L14N+R16T, E15T, E15N+E17T; R16N+C18S/T, M19N+E21T; E20N+K22T, K22N, S24N+E26T; F25N+E27T; E26N+A28T; E27N+R29T; A28N+E30T; R29N+V31S/T, E30N+F32T; V31N+E33T; F32N+N34T, E33N, T35N+R37S/T, E36T; E36N; R37N, T39N+F41S/T, E40N+W42T, F41N+K43S/T, W42N+Q44S/T, K43N+Y45T; Q44N+V46S/T, Y45N+D47T, V46N+G48S/T, D47N+D49S/T, G48N+Q50S/T, D49N+C51S/T, Q50N+E52S/T, E52N+N54T, S53N+P55S/T, C56S/T, L57N+G59S/T, G59N+S61T; G60S/T, S61N+K63S/T, K63N+D65S/T, D65N+N67S/T, I66N+S68S/T, Y69S/T, Y69N+C71S/T, S68N+E70S/T, E70N+W72S/T, W72N+P74S/T, P74N+G76S/T, F75N, G76N+E78T, E78N+K80T, F77T, F77N+G79S/T, G79N+N81S/T, K80N+C82S/T, E83S/T, E83N+D85S/T, L84N+V86S/T, D85N, V86A, V86N+C88S/T, T87N+N89S/T, I90N+N92S/T, K91S/T, I90N+N92S/T, K91N+G93S/T, R94S/T, R94N+E96S/T, K100N, A103S/T, S102N+D104S/T, A103N+N105S/T, D104N+K106S/T, V107S/T, K106N+V108S/T, V108N+V110S/T, S111N, E113N+Y115S/T, G114N+R116S/T, R116N+A118S/T, E119N+Q121S/T, K122S/T, Q121N+S123S/T, K122N+C124S/T S123N+E125S/T, E125N+A125S/T, P126N+V128S/T, A127N+P129T, V128N+F130S/T, P129N+P131S/T, F130N+C132S/T, R134N, V135N+V137S/T, S136N, S138N, V137N+Q139T; Q139N, T140N+L142S/T, S141N+L143S/T, K142N, A146N+A148S/T, E147N+V149S/T, T148N+F150S/T, V149N+P151S/T, F150N+D152S/T, P151N+V153S/T, D152N+D154S/T, V153N+Y155S/T, D154N+V156S/T, Y155N+N157S/T, V156N, S158N+E160S/T, T159N+A161S/T, E160N+E162S/T, A161N, E162N+I164S/T, T163N+L165S/T, I164N+D166S/T, L165N+N167S/T, D166N+I168S/T, I168N+Q170S/T, T169N, Q170N, S171N+Q173S/T, T172N, Q173N+F175S/T, S174N+N176S/T, F175N+D177S/T, F178S/T, D177N, D177E, F178N+R180S/T, T179N+V181S/T, R180N+V182S/T, G183+E185S/T, G184N+D186T, E185N+A187S/T, D186N+K188S/T, A187N+P189T, K188N+G190S/T, P189N+Q181S/T, G200N+V202T, K201N+D203S/T, K201T, V202N+A204S/T, D203N+F205S/T, E213N+W215S/T, K214T, V223T, E224N+G226S/T, T225N+V227S/T, G226N+K228S/T, V227N+I229T, K228N, H236N+I238T; I238N+E240T; E239N, E240N+E242S/T, E242N, T241N+H243S/T, H243N+E245S/T, K247N+N249S/T, V250N+R252T, I251S/T, I251N+I253S/T, R252N+I254S/T, I253N+P255S/T, P255N+H257S/T, H257N+Y259S/T, N260S/T, A262S/T, A261N+I263S/T, A262N+N264S/T, I263N+K265S/T, K265N+N267S/T, A266N+H268S/T, D276N+P278S/T, P278N+V280S/T, E277N+L279S/T, V280N+N282S/T, Y284S/T, S283N+V285S/T, Y284N, D292N+K294S/T, K293N+Y295 S/T, E294N, F299S/T, I298N+L300S/T, K301N+G303S/T, F302N, G303N+G305S/T, S304N+Y306S/T, Y306N+S308S/T, R312N+F314S/T, V313N+H315T, F314N+K316S/T, H315N+G317S/T, K316N+R138S/T, G317N, R318N+A320S/T, S319N+L321S/T, A320N+V322T, L321N+L323 S/T, V322N+Q324S/T, Y325N+R327S/T, R327N+P329S/T, P329N+V331S/T, L330N+D332S/T, D332N+A334S/T, R333N, A334N+C336S/T, T335N+L337S/T, L337N, R338N, S339N+K341T, T340N+F342T; K341N, F342N+I344S/T, T343N+Y345S/T, Y345N+N347S/T, M348S/T, G352N+H354T, F353N, F353N+E355T, H354N+G356S/T, H354V, H354I, E355T, E355N+G357S/T, G356N+R358T, G357N+D359S/T, R358N, Q362N+D364S/T, V370N; T371V; T371I; E372T, E372N+E374S/T, E374N, G375N, W385N+E387T; G386N+E388T, E388N+A390S/T, A390N+K392T, M391N+G393 S/T, K392N+K394S/T, K392V, G393T, G393N+Y395S/T, K394N+G396S/T, R403N+V405S/T, I408S/T, K409N+K411S/T, E410N, K411N+K413S/T, and K413N.

e. Modifications to Increase Resistance to Proteases

Modified FIX polypeptides provided herein also can contain additional modifications that result in increased resistance of the polypeptide to proteases. For example, amino acid substitutions can be made that remove one or more potential proteolytic cleavage sites. The modified FIX polypeptides can thus be made more resistant to proteases, thereby increasing the stability and half-life of the modified polypeptide.

Examples of additional modifications that can be included in the modified FIX polypeptides provided herein to increase resistance to proteases include, but are not limited to, those described in U.S. Patent Publication No. 2008/0102115 and International Application Publication No. WO 2007/149406. Exemplary modifications that can be included in a modified FIX polypeptide provided herein to increase protease resistance include, but are not limited to, Y1H, Y1I, S3Q, S3H, S3N, G4Q, G4H, G4N, K5N, K5Q, L6I, L6V, E7Q, E7H, E7N, E8Q, E8H, E8N, F9I, F9V, V10Q, V10H, V10N, G12Q, G12H, G12N, L141, L14V, E15Q, E15H, E15N, R16H, R16Q, E17Q, E17H, E17N, M19I, M19V, E20Q, E20H, E20N, E21Q, E21H, E21N, K22N, K22Q, S24Q, S24H, S24N, F251, F25V, E26Q, E26H, E26N, E27Q, E27H, E27N, A28Q, A28H, A28N, R29H, R29Q, E30Q, E30H, E30N, V31Q, V31H, V31N, F32I, F32V, E33Q, E33H, E33N, T35Q, T35H, T35N, E36Q, E36H, E36N, R37H, R37Q, T38Q, T38H, T38N, T39Q, T39H, T39N, E40Q, E40H, E40N, F41I, F41V, W42S, W42H, K43N, K43Q, Y45H, Y45I, V46Q, V46H, V46N, D47N, D47Q, G48Q, G48H, G48N, D49N, D49Q, E52Q, E52H, E52N, S53Q, S53H, S53N, P55A, P55S, L57I, L57V, N58Q, N58S, G59Q, G59H, G59N, G60Q, G60H, G60N, S61Q, S61H, S61N, K63N, K63Q, D64N, D64Q, D65N, D65Q, I66Q, I66H, I66N, S68Q, S68H, S68N, Y69H, Y69I, E70Q, E70H, E70N, W72S, W72H, P74A, P74S, F75I, F75V, G76Q, G76H, G76N, F77I, F77V, E78Q, E78H, E78N, G79Q, G79H, G79N, K80N, K80Q, E83Q, E83H, E83N, L84I, L84V, D85N, D85Q, V86Q, V86H, V86N, T87Q, T87H, T87N, I90Q, I90H, I90N, K91N, K91Q, N92Q, N92S, G93Q, G93H, G93N, R94H, R94Q, E96Q, E96H, E96N, F98I, F98V, K100N, K100Q, S102Q, S102H, S102N, A103Q, A103H, A103N, D104N, D104Q, K106N, K106Q, V107Q, V107H, V107N, V108Q, V108H, V108N, S110Q, S110H, S110N, T112Q, T112H, T112N, E113Q, E113H, E113N, G114Q, G114H, G114N, Y115H, Y115I, R116H, R116Q, L117I, L117V, A118Q, A118H, A118N, E119Q, E119H, E119N, K122N, K122Q, S123Q, S123H, S123N, E125Q, E125H, E125N, P126A, P126S, A127Q, A127H, A127N, V128Q, V128H, V128N, P129A, P129S, P131A, P131S, G133Q, G133H, G133N, R134H, R134Q, V135Q, V135H, V135N, S136Q, S136H, S136N, V137Q, V137H, V137N, S138Q, S138H, S138N, T140Q, T140H, T140N, S141Q, S141H, S141N, K142N, K142Q, L143I, L143V, T144Q, T144H, T144N, R145H, R145Q, A146Q, A146H, A146N, E147Q, E147H, E147N, T148Q, T148H, T148N, V149Q, V149H, V149N, P151A, P151S, D152N, D152Q, V153Q, V153H, V153N, D154N, D154Q, Y155H, Y155I, V156Q, V156H, V156N, S158Q, S158H, S158N, T159Q, T159H, T159N, E160Q, E160H, E160N, A161Q, A161H, A161N, E162Q, E162H, E162N, T163Q, T163H, T163N, I164Q, I164H, I164N, L165I, L165V, L165Q, L165H, D166N, D166Q, I168Q, I168H, I168N, T169Q, T169H, T169N, S171Q, S171H, S171N, T172Q, T172H, T172N, S174Q, S174H, S174N, F175I, F175V, F175H, D177N, D177Q, F178I, F178V, F178H, T179Q, T179H, T179N, R180H, R180Q, V181Q, V181H, V181N, V182Q, V182H, V182N, G183Q, G183H, G183N, G184Q, G184H, G184N, E185Q, E185H, E185N, D186N, D186Q, A187Q, A187H, A187N, K188N, K188Q, P189A, P189S, G190Q, G190H, G190N, F192I, F192V, F192IH, P193A, P193S, W194S, W194H, W194I, V196Q, V196H, V196N, V197Q, V197H, V197N, L198I, L198V, L198Q, L198H, N199Q, N199S, G200Q, G200H, G200N, K201N, K201Q, V202Q, V202H, V202N, D203N, D203Q, A204Q, A204H, A204N, F205I, F205V, G207Q, G207H, G207N, G208Q, G208H, G208N, S209Q, S209H, S209N, I210Q, I210H, I210N, V211Q, V211H, V211N, E213Q, E213H, E213N, K214N, K214Q, W215S, W215H, I216Q, I216H, I216N, V217Q, V217H, V217N, T218Q, T218H, T218N, A219Q, A219H, A219N, A220Q, A220H, A220N, V223Q, V223H, V223N, E224Q, E224H, E224N, T225Q, T225H, T225N, G226Q, G226H, G226N, V227Q, V227H, V227N, K228N, K228Q, I229Q, I229H, I229N, T230Q, T230H, T230N, V231Q, V231H, V231N, V232Q, V232H, V232N, A233Q, A233H, A233N, G234Q, G234H, G234N, E235Q, E235H, E235N, I238Q, I238H, I238N, E239Q, E239H, E239N, E240Q, E240H, E240N, T241Q, T241H, T241N, E242Q, E242H, E242N, T244Q, T244H, T244N, E245Q, E245H, E245N, K247N, K247Q, R248H, R248Q, V250Q, V250H, V250N, I251Q, I251H, I251N, R252H, R252Q, I253Q, I253H, I253N, I254Q, I254H, I254N, P255A, P255S, Y259H, Y259I, A261Q, A261H, A261N, A262Q, A262H, A262N, I263Q, I263H, I263N, K265N, K265Q, Y266H, Y266I, D269N, D269Q, I270Q, I270H, I270N, A271Q, A271H, A271N, L272I, L272V, L273I, L273V, E274Q, E274H, E274N, L275I, L275V, D276N, D276Q, E277Q, E277H, E277N, P278A, P278S, L279I, L279V, V280Q, V280H, V280N, L281I, L281V, S283Q, S283H, S283N, Y284H, Y284I, V285Q, V285H, V285N, T286Q, T286H, T286N, P287A, P287S, I288Q, I288H, I288N, I290Q, I290H, I290N, A291Q, A291H, A291N, D292N, D292Q, K293N, K293Q, E294Q, E294H, E294N, Y295H, Y295I, T296Q, T296H, T296N, I298Q, I298H, I298N, F299I, F299V, L300I, L300V, K301N, K301Q, F302I, F302V, G303Q, G303H, G303N, S304Q, S304H, S304N, G305Q, G305H, G305N, Y306H, Y306I, V307Q, V307H, V307N, S308Q, S308H, S308N, G309Q, G309H, G309N, W310S, W310H, G311Q, G311H, G311N, R312H, R312Q, V313Q, V313H, V313N, F314I, F314V, K316N, K316Q, G317Q, G317H, G317N, R318H, R318Q, S319Q, S319H, S319N, A320Q, A320H, A320N, L321I, L321V, V322Q, V322H, V322N, L323I, L323V, Y325H, Y325I, L326I, L326V, R327H, R327Q, V328Q, V328H, V328N, P329A, P329S, L330I, L330V, V331Q, V331H, V331N, D332N, D332Q, R333H, R333Q, A334Q, A334H, A334N, T335Q, T335H, T335N, L337I, L337V, R338H, R338Q, S339Q, S339H, S339N, T340Q, T340H, T340N, K341N, K341Q, F342I, F342V, T343Q, T343H, T343N, I344Q, I344H, I344N, Y345H, Y345I, M348I, M348V, F349I, F349V, A351Q, A351H, A351N, G352Q, G352H, G352N, F353I, F353V, E355Q, E355H, E355N, G356Q, G356H, G356N, G357Q, G357H, G357N, R358H, R358Q, D359N, D359Q, S360Q, S360H, S360N, G363Q, G363H, G363N, D364N, D364Q, S365Q, S365H, S365N, G366Q, G366H, G366N, G367Q, G367H, G367N, P368A, P368S, V370Q, V370H, V370N, T371Q, T371H, T371N, E372Q, E372H, E372N, V373Q, V373H, V373N, E374Q, E374H, E374N, G375Q, G375H, G375N, T376Q, T376H, T376N, S377Q, S377H, S377N, F378I, F378V, L379I, L379V, T380Q, T380H, T380N, G381Q, G381H, G381N, I382Q, I382H, I382N, I383Q, I383H, I383N, S384Q, S384H, S384N, W385S, W385H, G386Q, G386H, G386N, E387Q, E387H, E387N, E388Q, E388H, E388N, A390Q, A390H, A390N, M391I, M391V, K392N, K392Q, G393Q, G393H, G393N, K394N, K394Q, Y395H, Y395I, G396Q, G396H, G396N, I397Q, I397H, I397N, Y398H, Y398I, T399Q, T399H, T399N, K400N, K400Q, V401Q, V401H, V401N, S402Q, S402H, S402N, R403H, R403Q, Y404H, Y404I, V405Q, V405H, V405N, W407S, W407H, I408Q, I408H, I408N, K409N, K409Q, E410Q, E410H, E410N, K411N, K411Q, T412Q, T412H, T412N, K413N, K413Q, L414I, L414V, T415Q, T415H, and T415N (numbering corresponding to a mature FIX polypeptide set forth in SEQ ID NO:3).

f. Modifications to Reduce Immunogenicity

Further modifications to a modified FIX polypeptide provided herein can include modifications of at least one amino acid residue resulting in a substantial reduction in activity of or elimination of one or more T-cell epitopes from the protein, i.e., deimmunization of the polypeptide. One or more amino acid modifications at particular positions within any of the MHC class II ligands can result in a deimmunized FIX polypeptide with reduced immunogenicity when administered as a therapeutic to a subject, such as for example, a human subject. For example, any one or more modifications disclosed in U.S. Patent Publication No. 2004/0254106 can be included in the modified FIX polypeptide provided herein to reduce immunogenicity.

Exemplary amino acid modifications that can contribute to reduced immunogenicity of a FIX polypeptide include any one or more amino acid modifications corresponding to any one or more of the following modifications: Y1A, Y1C, Y1D, Y1E, Y1G, Y1H, Y1K, Y1N, Y1P, Y1Q, Y1R, Y1S, Y1T, S3T, L6A, L6C, L6D, L6E, L6G, L6H, L6K, L6N, L6P, L6Q, L6R, L6S, L6T, L6M, F9A, F9C, F9D, F9E, F9G, F9H, F9K, F9N, F9P, F9Q, F9R, F9S, F9T, F9I, F9M, F9W, V10A, V10C, V10D, V10E, V10G, V10H, V10K, V10N, V10P, V10Q, V10R, V10S, V10T, V10F, V10I, V10M, V10W, V10Y, Q11A, Q11C, Q11G, Q11P, G12D, G12E, G12G, G12H, G12K, G12N, G12P, G12Q, G12R, G12S, G12T, N13A, N13C, N13G, N13H, N13P, N13T, L14A, L14C, L14D, L14E, L14G, L14H, L14K, L14N, L14P, L14Q, L14R, L14S, L14T, L14F, L14I, L14M, L14V, L14W, L14Y, E15D, E15H, E15P, R16A, R16C, R16G, R16P, R16T, E17A, E17C, E17G, E17P, E17T, C18D, C18E, C18G, C18H, C18K, C18N, C18P, C18Q, C18R, C18S, C18T, M19A, M19C, M19D, M19E, M19G, M19H, M19K, M19N, M19P, M19Q, M19R, M19S, M19T, M19F, M19I, M19M, M19V, M19W, M19Y, E20A, E20C, E20G, E20P, E20T, E21A, E21C, E21G, E21P, K22H, K22P, K22T, S24H, S24P, F25A, F25C, F25D, F25E, F25G, F25H, F25K, F25N, F25P, F25Q, F25R, F25S, F25T, F25I, F25M, F25W, F25Y, E26A, E26C, E26G, E26P, E27A, E27C, E27G, E27H, E27P, E27S, E27T, A28C, A28D, A28E, A28G, A28H, A28K, A28N, A28P, A28Q, A28R, A28S, A28T, R29A, R29C, R29G, R29P, E30D, E30H, E30P, V31A, V31C, V31D, V31E, V31G, V31H, V31K, V31N, V31P, V31Q, V31R, V31S, V31T, V31F, V31I, V31W, V31Y, F32A, F32C, F32D, F32E, F32G, F32H, F32K, F32N, F32P, F32Q, F32R, F32S, F32T, E33H, E33N, E33P, E33Q, E33S, E33T, T35A, T35C, T35G, T35P, F41A, F41C, F41D, F41E, F41G, F41H, F41K, F41N, F41P, F41Q, F41R, F41S, F41T, F41M, F41W, F41Y, W42A, W42C, W42D, W42E, W42G, W42H, W42K, W42N, W42P, W42Q, W42R, W42S, W42T, K43A, K43C, K43G, K43P, Q44P, Q44T, Q44, Y45A, Y45C, Y45D, Y45E, Y45G, Y45H, Y45K, Y45N, Y45P, Y45Q, Y45R, Y45S, Y45T, V46A, V46C, V46D, V46E, V46G, V46H, V46K, V46N, V46P, V46Q, V46R, V46S, V46T, V46F, V46I, V46M, V46W, V46Y, D47A, D47C, D47G, D47H, D47P, D47T, G48D, G48E, G48P, G48T, D49H, D49P, D49Q, D49T, Q50A, Q50C, Q50D, Q50G, Q50H, Q50P, Q50T, C51D, C51E, C51G, C51H, C51K, C51N, C51P, C51Q, C51R, C51S, C51T, E52P, E52T, S53A, S53C, S53G, S53H, S53P, S53T, N54H, N54P, N54T, L57A, L57C, L57D, L57E, L57G, L57H, L57K, L57N, L57P, L57Q, L57R, L57S, L57T, L57F, L57I, L57M, L57W, L57Y, G60C, G60D, G60H, G60P, G60T, C62D, C62H, C62P, K63T, D65H, D65T, I66A, I66C, I66D, I66E, I66G, I66H, I66K, I66N, I66P, I66Q, I66R, I66S, I66T, I66M, I66W, I66Y, Y69A, Y69C, Y69D, Y69E, Y69G, Y69H, Y69K, Y69N, Y69P, Y69Q, Y69R, Y69S, Y69T, C71H, C71P, W72A, W72C, W72D, W72E, W72G, W72H, W72K, W72N, W72P, W72Q, W72R, W72S, W72T, W72I, W72Y, F75A, F75C, F75D, F75E, F75G, F75H, F75K, F75N, F75P, F75Q, F75R, F75S, F75T, F77A, F77C, F77D, F77E, F77G, F77H, F77K, F77N, F77P, F77Q, F77R, F77S, F77T, L84A, L84C, L84D, L84E, L84G, L84H, L84K, L84N, L84P, L84Q, L84R, L84S, L84T, L84M, L84W, L84Y, V86A, V86C, V86D, V86E, V86G, V86H, V86K, V86N, V86P, V86Q, V86R, V86S, V86T, I90A, I90C, I90D, I90E, I90G, I90H, I90K, I90N, I90P, I90Q, I90R, I90S, I90T, I90M, I90W, K91A, K91C, K91G, K91P, N92A, N92C, N92G, N92P, N92T, G93D, G93E, G93H, G93K, G93N, G93P, G93Q, G93R, G93S, G93T, R94A, R94C, R94G, R94P, C95D, C95E, C95G, C95H, C95K, C95N, C95P, C95Q, C95R, C95S, C95T, E96P, E96T, Q97A, Q97C, Q97G, Q97P, F98A, F98C, F98D, F98E, F98G, F98H, F98K, F98N, F98P, F98Q, F98R, F98S, F98T, F98M, F98W, F98Y, K100A, K100C, K100G, K100P, N101H, N101T, A103D, A103E, A103H, A103K, A103N, A103P, A103Q, A103R, A103S, A103T, D104T, K106H, K106P, K106T, V107A, V107C, V107D, V107E, V107G, V107H, V107K, V107N, V107P, V107Q, V107R, V107S, V107T, V108A, V108C, V108D, V108E, V108G, V108H, V108K, V108N, V108P, V108Q, V108R, V108S, V108T, V108F, V108M, V108W, V108Y, S110A, S110C, S110G, S110P, C111D, C111E, C111H, C111K, C111N, C111P, C111Q, C111R, C111S, C111T, T112A, T112C, T112G, T112P, E113D, E113H, E113P, G114D, G114E, G114H, G114K, G114N, G114P, G114Q, G114R, G114S, G114T, Y115A, Y115C, Y115D, Y115E, Y115G, Y115H, Y115K, Y115N, Y115P, Y115Q, Y115R, Y115S, Y115T, Y115M, Y115W, R116P, R116T, L117A, L117C, L117D, L117E, L117G, L117H, L117K, L117N, L117P, L117Q, L117R, L117S, L117T, A118D, A118E, A118H, A118K, A118N, A118P, A118Q, A118R, A118S, A118T, N120D, N120H, N120P, Q121T, S123H, S123T, V128A, V128C, V128D, V128E, V128G, V128H, V128K, V128N, V128P, V128Q, V128R, V128S, V128T, F130A, F130C, F130D, F130E, F130G, F130H, F130K, F130N, F130P, F130Q, F130R, F130S, F130T, V135A, V135C, V135D, V135E, V135G, V135H, V135K, V135N, V135P, V135Q, V135R, V135S, V135T, V135W, V135Y, V137A, V137C, V137D, V137E, V137G, V137H, V137K, V137N, V137P, V137Q, V137R, V137S, V137T, V137M, V137W, V137Y, S138H, S138T, T140D, T140H, S141T, K142H, K142P, L143A, L143C, L143D, L143E, L143G, L143H, L143K, L143N, L143P, L143Q, L143R, L143S, L143T, L143F, L143I, L143M, L143V, L143W, L143Y, R145H, R145P, R145T, A146P, A146T, T148H, T148P, V149A, V149C, V149D, V149E, V149G, V149H, V149K, V149N, V149P, V149Q, V149R, V149S, V149T, V149F, V149I, V149M, V149W, V149Y, F150A, F150C, F150D, F150E, F150G, F150H, F150K, F150N, F150P, F150Q, F150R, F150S, F150T, F150M, F150W, F150Y, D152A, D152C, D152G, D152P, D152S, D152T, V153A, V153C, V153D, V153E, V153G, V153H, V153K, V153N, V153P, V153Q, V153R, V153S, V153T, V153F, V153I, V153M, V153W, V153Y, D154A, D154C, D154G, D154P, D154Q, D154S, Y155A, Y155C, Y155D, Y155E, Y155G, Y155H, Y155K, Y155N, Y155P, Y155Q, Y155R, Y155S, Y155T, Y155M, Y155V, Y155W, V156A, V156C, V156D, V156E, V156G, V156H, V156K, V156N, V156P, V156Q, V156R, V156S, V156T, V156I, V156M, V156W, V156Y, N157A, N157C, N157G, N157H, N157P, N157Q, N157T, S158H, S158P, S158T, T159A, T159C, T159G, T159P, E160A, E160C, E160G, E160P, A161C, A161D, A161E, A161H, A161K, A161N, A161P, A161Q, A161R, A161S, A161T, E162P, E162T, T163A, T163C, T163G, T163P, I164A, I164C, I164D, I164E, I164G, I164H, I164K, I164N, I164P, I164Q, I164R, I164S, I164T, L165A, L165C, L165D, L165E, L165G, L165H, L165K, L165N, L165P, L165Q, L165R, L165S, L165T, L165M, L165W, L165Y, I168A, I168C, I168D, I168E, I168G, I168H, I168K, I168N, I168P, I168Q, I168R, I168S, I168T, F175A, F175C, F175D, F175E, F175G, F175H, F175K, F175N, F175P, F175Q, F175R, F175S, F175T, F178A, F178C, F178D, F178E, F178G, F178H, F178K, F178N, F178P, F178Q, F178R, F178S, F178T, F178M, F178W, F178Y, T179A, T179C, T179G, T179P, R180A, R180C, R180D, R180G, R180H, R180P, V181A, V181C, V181D, V181E, V181G, V181H, V181K, V181N, V181P, V181Q, V181R, V181S, V181T, V181F, V181I, V181M, V181W, V181Y, V182A, V182C, V182D, V182E, V182G, V182H, V182K, V182N, V182P, V182Q, V182R, V182S, V182T, V182F, V182I, V182M, V182W, V182Y, G183D, G183E, G183H, G183K, G183N, G183P, G183Q, G183S, G183T, G184D, G184E, G184H, G184K, G184N, G184P, G184Q, G184R, G184S, G184T, E185A, E185C, E185G, E185H, E185P, E185T, D186A, D186C, D186G, D186H, D186P, D186T, A187C, A187D, A187E, A187G, A187H, A187K, A187N, A187P, A187Q, A187R, A187S, A187T, K188A, K188C, K188G, K188H, K188P, K188T, G190D, G190E, G190H, G190K, G190N, G190P, G190Q, G190R, G190S, G190T, F192A, F192C, F192D, F192E, F192G, F192H, F192K, F192N, F192P, F192Q, F192R, F192S, F192T, F192W, F192Y, W194A, W194C, W194D, W194E, W194G, W194H, W194K, W194N, W194P, W194Q, W194R, W194S, W194T, Q195H, Q195P, Q195T, V196A, V196C, V196D, V196E, V196G, V196H, V196K, V196N, V196P, V196Q, V196R, V196S, V196T, V196F, V1961, V196M, V196W, V196Y, V197A, V197C, V197D, V197E, V197G, V197H, V197K, V197N, V197P, V197Q, V197R, V197S, V197T, V197F, V197I, V197M, V197W, V197Y, L198A, L198C, L198D, L198E, L198G, L198H, L198K, L198N, L198P, L198Q, L198R, L198S, L198T, L198I, L198Y, N199A, N199C, N199G, N199H, N199P, N199S, N199T, G200P, G200T, K201A, K201C, K201D, K201E, K201G, K201H, K201N, K201P, K201Q, K201S, K201T, V202A, V202C, V202D, V202E, V202G, V202H, V202K, V202N, V202P, V202Q, V202R, V202S, V202T, V202F, V202I, V202M, V202W, V202Y, D203A, D203C, D203G, D203P, D203T, A204C, A204D, A204E, A204G, A204H, A204K, A204N, A204P, A204Q, A204R, A204S, A204T, F205A, F205C, F205D, F205E, F205G, F205H, F205K, F205N, F205P, F205Q, F205R, F205S, F205T, F205M, F205V, F205W, F205Y, G207H, G207P, G208C, G208D, G208E, G208H, G208K, G208N, G208P, G208Q, G208R, G208S, G208T, S209A, S209C, S209G, S209P, I210A, I210C, I210D, I210E, I210G, I210H, I210K, I210N, I210P, I210Q, I210R, I210S, I210T, I210F, I210W, I210Y, V211A, V211C, V211D, V211E, V211G, V211H, V211K, V211N, V211P, V211Q, V211R, V211S, V211T, V211F, V211I, V211M, V211W, N212A, N212C, N212G, N212P, E213H, E213P, E213S, E213T, K214T, W215A, W215C, W215D, W215E, W215G, W215H, W215K, W215N, W215P, W215Q, W215R, W215S, W215T, I216A, I216C, I216D, I216E, I216G, I216H, I216K, I216N, I216P, I216Q, I216R, I216S, I216T, V217A, V217C, V217D, V217E, V217G, V217H, V217K, V217N, V217P, V217Q, V217R, V217S, V217T, V2171, V217Y, A219H, A219P, A219T, V223A, V223C, V223D, V223E, V223G, V223H, V223K, V223N, V223P, V223Q, V223R, V223S, V223T, V223M, V223W, V223Y, G226P, V227A, V227C, V227D, V227E, V227G, V227H, V227K, V227N, V227P, V227Q, V227R, V227S, V227T, V227F, V2271, V227M, V227W, V227Y, K228A, K228C, K228G, K228H, K228P, I229A, I229C, I229D, I229E, I229G, I229H, I229K, I229N, I229P, I229Q, I229R, I229S, I229T, I229M, I229W, I229Y, T230A, T230C, T230G, T230P, V231A, V231C, V231D, V231E, V231G, V231H, V231K, V231N, V231P, V231Q, V231R, V231S, V231T, V232A, V232C, V232D, V232E, V232G, V232H, V232K, V232N, V232P, V232Q, V232R, V232S, V232T, V232F, V2321, V232M, V232W, V232Y, A233C, A233D, A233E, A233G, A233H, A233K, A233N, A233P, A233Q, A233R, A233S, A233T, A233V, G234D, G234E, G234H, G234K, G234N, G234P, G234Q, G234R, G234S, G234T, E235H, E235N, E235P, E235Q, E235S, E235T, H236A, H236C, H236G, H236P, N237A, N237C, N237G, N237P, N237T, I238A, I238C, I238D, I238E, I238G, I238H, I238K, I238N, I238P, I238Q, I238R, I238S, I238T, E239A, E239C, E239G, E239P, E240H, E240T, V250A, V250C, V250D, V250E, V250G, V250H, V250K, V250N, V250P, V250Q, V250R, V250S, V250T, V250M, V250W, V250Y, I251A, I251C, I251D, I251E, I251G, I251H, I251K, I251N, I251P, I251Q, I251R, I251S, I251T, I253A, I253C, I253D, I253E, I253G, I253H, I253K, I253N, I253P, I253Q, I253R, I253S, I253T, I253M, I253W, I253Y, I254A, I254C, I254D, I254E, I254G, I254H, I254K, I254N, I254P, I254Q, I254R, I254S, I254T, P255H, H256P, H256T, H257A, H257C, H257G, H257P, N258P, N258T, Y259A, Y259C, Y259D, Y259E, Y259G, Y259H, Y259K, Y259N, Y259P, Y259Q, Y259R, Y259S, Y259T, Y259M, Y259W, N260A, N260C, N260G, N260P, A261D, A261E, A261H, A261K, A261N, A261P, A261Q, A261R, A261S, A261T, A262C, A262D, A262E, A262G, A262H, A262K, A262N, A262P, A262Q, A262R, A262S, A262T, I263A, I263C, I263D, I263E, I263G, I263H, I263K, I263N, I263P, I263Q, I263R, I263S, I263T, I263M, I263V, I263W, I263Y, N264A, N264C, N264D, N264G, N264H, N264P, K265A, K265C, K265G, K265H, K265P, Y266A, Y266C, Y266D, Y266E, Y266G, Y266H, Y266K, Y266N, Y266P, Y266Q, Y266R, Y266S, Y266T, Y266M, Y266W, N267A, N267C, N267G, N267H, N267P, N267T, H268P, D269A, D269C, D269E, D269G, D269H, D269N, D269P, D269Q, D269S, D269T, I270A, I270C, I270D, I270E, I270G, I270H, I270K, I270N, I270P, I270Q, I270R, I270S, I270T, I270M, I270W, A271C, A271D, A271E, A271G, A271H, A271K, A271N, A271P, A271Q, A271R, A271S, A271T, L272A, L272C, L272D, L272E, L272G, L272H, L272K, L272N, L272P, L272Q, L272R, L272S, L272T, L272F, L273A, L273C, L273D, L273E, L273G, L273H, L273K, L273N, L273P, L273Q, L273R, L273S, L273T, L273F, L273I, L273M, L273V, L273W, L273Y, E274A, E274C, E274G, E274P, E274T, L275A, L275C, L275D, L275E, L275G, L275H, L275K, L275N, L275P, L275Q, L275R, L275S, L275T, L275W, L275Y, D276P, D276S, D276T, E277A, E277C, E277G, E277P, P278T, L279A, L279C, L279D, L279E, L279G, L279H, L279K, L279N, L279P, L279Q, L279R, L279S, L279T, L279I, L279Y, V280A, V280C, V280D, V280E, V280G, V280H, V280K, V280N, V280P, V280Q, V280R, V280S, V280T, V280F, V280I, V280W, V280Y, L281A, L281C, L281D, L281E, L281G, L281H, L281K, L281N, L281P, L281Q, L281R, L281S, L281T, L281F, L281I, L281V, L281W, L281Y, S283A, S283C, S283G, S283P, Y284A, Y284C, Y284D, Y284E, Y284G, Y284H, Y284K, Y284N, Y284P, Y284Q, Y284R, Y284S, Y284T, Y284M, V285A, V285C, V285D, V285E, V285G, V285H, V285K, V285N, V285P, V285Q, V285R, V285S, V285T, V285M, V285W, V285Y, T286A, T286C, T286G, T286P, I288A, I288C, I288D, I288E, I288G, I288H, I288K, I288N, I288P, I288Q, I288R, I288S, I288T, C289D, C289H, C289P, I290A, I290C, I290D, I290E, I290G, I290H, I290K, I290N, I290P, I290Q, I290R, I290S, I290T, I290Y, A291D, A291E, A291H, A291K, A291N, A291P, A291Q, A291R, A291S, A291T, D292A, D292C, D292G, D292P, D292T, K293H, K293P, K293T, Y295A, Y295C, Y295D, Y295E, Y295G, Y295H, Y295K, Y295N, Y295P, Y295Q, Y295R, Y295S, Y295T, Y295W, T296A, T296C, T296G, T296P, N297A, N297C, N297G, N297P, I298A, I298C, I298D, I298E, I298G, I298H, I298K, I298N, I298P, I298Q, I298R, I298S, I298T, F299A, F299C, F299D, F299E, F299G, F299H, F299K, F299N, F299P, F299Q, F299R, F299S, F299T, L300A, L300C, L300D, L300E, L300G, L300H, L300K, L300N, L300P, L300Q, L300R, L300S, L300T, L300F, L300I, L300M, L300V, L300W, L300Y, K301A, K301C, K301G, K301P, K301T, F302A, F302C, F302D, F302E, F302G, F302H, F302K, F302N, F302P, F302Q, F302R, F302S, F302T, G303H, G303P, G303T, S304A, S304C, S304G, S304P, S304T, G305D, G305E, G305H, G305N, G305P, G305Q, G305S, G305T, Y306A, Y306C, Y306D, Y306E, Y306G, Y306H, Y306K, Y306N, Y306P, Y306Q, Y306R, Y306S, Y306T, V307A, V307C, V307D, V307E, V307G, V307H, V307K, V307N, V307P, V307Q, V307R, V307S, V307T, S308P, S308T, W310A, W310C, W310D, W310E, W310G, W310H, W310K, W310N, W310P, W310Q, W310R, W310S, W310T, G311H, V313A, V313C, V313D, V313E, V313G, V313H, V313K, V313N, V313P, V313Q, V313R, V313S, V313T, F314A, F314C, F314D, F314E, F314G, F314H, F314K, F314N, F314P, F314Q, F314R, F314S, F314T, F314M, F314W, F314Y, H315A, H315C, H315G, H315P, K316A, K316C, K316G, K316P, G317C, G317D, G317E, G317H, G317K, G317N, G317P, G317Q, G317R, G317S, G317T, R318A, R318C, R318G, R318P, S319D, S319H, S319N, S319P, S319Q, A320C, A320D, A320E, A320G, A320H, A320K, A320N, A320P, A320Q, A320R, A320S, A320T, L321A, L321C, L321D, L321E, L321G, L321H, L321K, L321N, L321P, L321Q, L321R, L321S, L321T, V322A, V322C, V322D, V322E, V322G, V322H, V322K, V322N, V322P, V322Q, V322R, V322S, V322T, V322W, V322Y, L323A, L323C, L323D, L323E, L323G, L323H, L323K, L323N, L323P, L323Q, L323R, L323S, L323T, L323F, L323I, L323M, L323V, L323W, L323Y, Q324A, Q324C, Q324G, Q324P, Y325A, Y325C, Y325D, Y325E, Y325G, Y325H, Y325K, Y325N, Y325P, Y325Q, Y325R, Y325S, Y325T, Y325W, L326A, L326C, L326D, L326E, L326G, L326H, L326K, L326N, L326P, L326Q, L326R, L326S, L326T, L326F, L326I, L326M, L326V, L326W, L326Y, R327A, R327C, R327G, R327H, R327P, V328A, V328C, V328D, V328E, V328G, V328H, V328K, V328N, V328P, V328Q, V328R, V328S, V328T, V328F, V328I, V328M, V328W, V328Y, L330A, L330C, L330D, L330E, L330G, L330H, L330K, L330N, L330P, L330Q, L330R, L330S, L330T, L330F, L330I, L330V, L330W, L330Y, V331A, V331C, V331D, V331E, V331G, V331H, V331K, V331N, V331P, V331Q, V331R, V331S, V331T, V331F, V331I, V331M, V331W, V331Y, D332A, D332C, D332G, D332P, R333A, R333C, R333D, R333E, R333G, R333H, R333N, R333P, R333Q, R333R, R333S, R333T, A334C, A334D, A334E, A334G, A334H, A334K, A334N, A334P, A334Q, A334R, A334S, A334T, T335A, T335C, T335G, T335P, C336D, C336E, C336H, C336K, C336N, C336P, C336Q, C336R, C336S, C336T, L337A, L337C, L337D, L337E, L337G, L337H, L337K, L337N, L337P, L337Q, L337R, L337S, L337T, R338A, R338C, R338G, R338P, S339P, S339T, K341A, K341C, K341G, K341P, F342A, F342C, F342D, F342E, F342G, F342H, F342K, F342N, F342P, F342Q, F342R, F342S, F342T, F342M, F342W, T343A, T343C, T343G, T343P, I344A, I344C, I344D, I344E, I344G, I344H, I344K, I344N, I344P, I344Q, I344R, I344S, I344T, Y345A, Y345C, Y345D, Y345E, Y345G, Y345H, Y345K, Y345N, Y345P, Y345Q, Y345R, Y345S, Y345T, Y345M, Y345W, N346A, N346C, N346G, N346P, N347H, N347P, M348A, M348C, M348D, M348E, M348G, M348H, M348K, M348N, M348P, M348Q, M348R, M348S, M348T, F349A, F349C, F349D, F349E, F349G, F349H, F349K, F349N, F349P, F349Q, F349R, F349S, F349T, F349I, F349M, F349W, F349Y, C350D, C350H, C350P, C350T, A351E, A351H, A351N, A351P, A351Q, A351R, A351S, A351T, G352A, G352C, G352P, F353A, F353C, F353D, F353E, F353G, F353H, F353K, F353N, F353P, F353Q, F353R, F353S, F353T, F353I, F353M, F353W, H354A, H354C, H354G, H354P, E355A, E355C, E355D, E355G, E355H, E355K, E355N, E355P, E355Q, E355S, E355T, G356D, G356E, G356H, G356K, G356N, G356P, G356Q, G356R, G356S, G356T, G357D, G357E, G357H, G357K, G357N, G357P, G357Q, G357R, G357S, G357T, R358D, R358E, R358H, R358K, R358N, R358P, R358Q, R358R, R358S, R358T, D359A, D359C, D359G, D359P, D359Q, D359S, D359T, S360A, S360C, S360G, S360P, C361D, C361E, C361H, C361K, C361N, C361P, C361Q, C361R, C361S, C361T, V370A, V370C, V370D, V370E, V370G, V370H, V370K, V370N, V370P, V370Q, V370R, V370S, V370T, V370W, V370Y, V373A, V373C, V373D, V373E, V373G, V373H, V373K, V373N, V373P, V373Q, V373R, V373S, V373T, V373F, V373I, V373M, V373W, E374A, E374C, E374G, E374P, G375H, S377A, S377C, S377G, S377P, F378A, F378C, F378D, F378E, F378G, F378H, F378K, F378N, F378P, F378Q, F378R, F378S, F378T, F378W, L379A, L379C, L379D, L379E, L379G, L379H, L379K, L379N, L379P, L379Q, L379R, L379S, L379T, L379I, L379M, L379W, L379Y, T380A, T380C, T380G, T380P, G381D, G381E, G381H, G381K, G381N, G381P, G381Q, G381R, G381S, G381T, I382A, I382C, I382D, I382E, I382G, I382H, I382K, I382N, I382P, I382Q, I382R, I382S, I382T, I382M, I382W, I382Y, I383A, I383C, I383D, I383E, I383G, I383H, I383K, I383N, I383P, I383Q, I383R, I383S, I383T, S384A, S384C, S384G, S384P, W385A, W385C, W385D, W385E, W385G, W385H, W385K, W385N, W385P, W385Q, W385R, W385S, W385T, W385M, E387A, E387C, E387G, E387H, E387P, E387T, E388H, E388N, E388P, E388Q, E388T, A390C, A390D, A390E, A390G, A390H, A390K, A390N, A390P, A390Q, A390R, A390S, M391A, M391C, M391D, M391E, M391G, M391H, M391K, M391N, M391P, M391Q, M391R, M391S, M391T, M391F, M391I, M391W, M391Y, K392A, K392C, K392G, K392P, G393C, G393D, G393E, G393H, G393K, G393N, G393P, G393Q, G393R, G393S, G393T, Y395A, Y395C, Y395D, Y395E, Y395G, Y395H, Y395K, Y395N, Y395P, Y395Q, Y395R, Y395S, Y395T, Y398A, Y398C, Y398D, Y398E, Y398G, Y398H, Y398K, Y398N, Y398P, Y398Q, Y398R, Y398S, Y398T, K400H, V401A, V401C, V401D, V401E, V401G, V401H, V401K, V401N, V401P, V401Q, V401R, V401S, V401T, V401F, V401I, V401M, V401W, V401Y, S402A, S402C, S402G, S402P, R403A, R403C, R403G, R403P, R403T, Y404A, Y404C, Y404D, Y404E, Y404G, Y404H, Y404K, Y404N, Y404P, Y404Q, Y404R, Y404S, Y404T, V405A, V405C, V405D, V405E, V405G, V405H, V405K, V405N, V405P, V405Q, V405R, V405S, V405T, V405W, V405Y, N406F, N406H, N406I, N406L, N406P, N406W, N406Y, W407D, W407E, W407F, W407H, W407I, W407K, W407N, W407P, W407Q, W407R, W407S, W407T, W407Y, I408D, I408E, I408H, I408K, I408N, I408P, I408Q, I408R, I408S, I408T, K409F, K409H, K409I, K409P, K409T, K409V, K409W, K409Y, E410H, K411A, K411C, K411G, K411I, K411P, K411T, K411V, K411W, K411Y, or K413T, with numbering corresponding to a mature FIX polypeptide set forth in SEQ ID NO: 3.

g. Exemplary Combination Modifications

Provided herein are modified FIX polypeptides that have two or more modifications designed to affect one or more properties or activities of an unmodified FIX polypeptide. In some examples, the two or more modifications alter two or more properties or activities of the FIX polypeptide. The modifications can be made to the FIX polypeptides such that one or more of glycosylation, resistance to AT-III, resistance to AT-III/heparin, resistance to heparin, catalytic activity, binding to LRP, intrinsic activity, phospholipid binding and/or affinity, resistance to proteases, half-life and interaction with other factors or molecules, such as FVIIIa and FX, is altered. Typically, the two or more modifications are combined such that the resulting modified FIX polypeptide has increased coagulant activity, increased duration of coagulant activity, and/or an enhanced therapeutic index compared to an unmodified FIX polypeptide. The modifications can include amino acid substitution, insertion or deletion. The increased coagulant activity, increased duration of coagulant activity, and/or an enhanced therapeutic index of the modified FIX polypeptide containing two or more modifications can be increased by at least or at least about 1%, 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, I90%, 200%, 300%, 400%, 500%, or more compared to the activity of the starting or unmodified FIXa polypeptide.

Provided herein are modified FIX polypeptides that contain two or more modifications that are introduced into an unmodified FIX polypeptide to alter one, two or more activities or properties. The modified FIX polypeptides can contain 2, 3, 4, 5, 6 or more modifications. For example, a modified FIX polypeptide provided herein can contain the modifications to increase glycosylation by incorporating a non-native glycosylation site into the primary sequence, such as amino acid substitutions D203N and F205T, to introduce a non-native glycosylation site at position 203, and a modification to increase resistance to AT-III/heparin, such as R338E (residues corresponding to a mature FIX polypeptide set forth in SEQ ID NO:3).

Modified FIX polypeptides provided herein can have two or more modifications selected solely from those set forth in Tables 3-9. In other examples, the modified FIX polypeptide contains two or more modifications where one or more modifications are selected from those set forth in Tables 3-9 and one or more modifications are additional modifications that are not set forth in Tables 3-9, such as, for example, modifications described in the art. In some examples, the one or more additional modifications can be selected from those set forth in Section D.3.a-f, above, such as those that result in increased catalytic activity, increased resistance to inhibitors, increased affinity and/or binding to platelets and phospholipids, increased protease resistance, decreased immunogenicity, and those that facilitate conjugation to moieties, such as PEG moieties.

Non-limiting exemplary combination modifications are provided in Table 12. These exemplary combination modifications include two or more modifications that are designed to alter two or more activities or properties of a FIX polypeptide, including, but not limited to, increased resistance to AT-III, increased resistance to AT-III/heparin, increased resistance to heparin, increased catalytic activity and altered glycosylation. Modified FIX polypeptides containing such combination modifications can have increased coagulant activity, increased duration of coagulant activity, and/or an enhanced therapeutic index. In Table 12 below, the sequence identifier (SEQ ID NO) is identified in which exemplary amino acid sequences of the modified FIX polypeptide are set forth.

TABLE 12

| Mutation | Mutation | SEQ |
|---|---|---|
| (Mature FIX | (Chymotrypsin | ID |
| Numbering) | Numbering) | NO. |
| R318Y/E410N | R150Y/E240N | 153 |
| R338E/E410N | R170E/E240N | 154 |
| R338E/R403E/E410N | R170E/R233E/E240N | 155 |
| D203N/F205T/K228N | D39N/F41T/K63N | 157 |
| D203N/F205T/E410N | D39N/F41T/E240N | 158 |
| D203N/F205T/R338E | D39N/F41T/R170E | 159 |
| D203N/F205T/R338A | D39N/F41T/R170A | 160 |
| D203N/F205T/R318Y | D39N/F41T/R150Y | 161 |
| D203N/F205T/R338E/R403E | D39N/F41T/R170E/R233E | 162 |
| K228N/E410N | K63N/E240N | 163 |
| K228N/R338E | K63N/R170E | 164 |
| K228N/R338A | K63N/R170A | 165 |
| K228N/R318Y | K63N/R150Y | 166 |
| K228N/R338E/R403E | K63N/R170E/R233E | 167 |
| R403E/E410N | R233E/E240N | 168 |
| R318Y/R338E/E410N | R150Y/R170E/E240N | 169 |
| K228N/R318Y/E410N | K63N/R150Y/E240N | 170 |
| R318Y/R403E/E410N | R150Y/R233E/E240N | 171 |
| R318Y/R338E/R403E/E410N | R150Y/R170E/R233E/E240N | 172 |
| D203N/F205T/R318Y/E410N | D39N/F41T/R150Y/E240N | 173 |
| F314N/K316S | F145N/K148S | 177 |
| A103N/N105S/K228N | A[103]N/N[105]S/K63N | 217 |
| D104N/K106S/K228N | D[104]N/K[106]S/K63N | 218 |
| K228N/I251S | K63N/I86S | 180 |
| A103N/N105S/I251S | A[103]N/N[105]S/I86S | 181 |
| D104N/K106S/I251S | D[104]N/K[106]S/I86S | 182 |
| A103N/N105S/R318Y/R338E/R403E/ | A[103]N/N[105]S/R150Y/R170E/ | 219 |
| E410N | R233E/E240N | |
| D104N/K106S/R318Y/R338E/R403E/ | D[104]N/K[106]S/R150Y/R170E/ | 220 |
| E410N | R233E/E240N | |
| K228N/R318Y/R338E/R403E/E410N | K63N/R150Y/R170E/R233E/E240N | 221 |
| I251S/R318Y/R338E/R403E/E410N | I86S/R150Y/R170E/R233E/E240N | 222 |
| D104N/K106S/I251S/R318Y/R338E/ | D[104]N/K[106]S/I86S/R150Y/ | 223 |
| R403E/E410N | R170E/R233E/E240N | |
| D104N/K106S/R318Y/R338E/E410N | D[104]N/K[106]S/R150Y/R170E/ | 224 |
| | E240N | |
| I251S/R318Y/R338E/E410N | I86S/R150Y/R170E/E240N | 225 |
| D104N/K106S/I251S/R318Y/R338E/ | D[104]N/K[106]S/I86S/R150Y/ | 226 |
| E410N | R170E/E240N | |
| A103N/N105S/K247N/N249S | A[103]N/N[105]S/K82N/N84S | 178 |
| D104N/K106S/K247N/N249S | D[104]N/K[106]S/K82N/N84S | 179 |
| K228N/K247N/N249S | K63N/K82N/N84S | 183 |
| A103N/N105S/Y155F | A[103]N/N[105]S/Y[155]F | 227 |
| D104N/K106S/Y155F | D[104]N/K[106]S/Y[155]F | 228 |
| Y155F/K228N | Y[155]F/K63N | 229 |
| Y155F/I251S | Y[155]F/I86S | 230 |
| Y155F/K247N/N249S | Y[155]F/K82N/N84S | 231 |
| A103N/N105S/K247N/N249S/R318Y/ | A[103]N/N[105]S/K82N/N84S/ | 232 |
| R338E/R403E/E410N | R150Y/R170E/R233E/E240N | |
| D104N/K106S/K247N/N249S/R318Y/ | D[104]N/K[106]S/K82N/N84S/ | 233 |
| R338E/R403E/E410N | R150Y/R170E/R233E/E240N | |
| K228N/K247N/N249S/R318Y/R338E/ | K63N/K82N/N84S/R150Y/R170E/R233E/E240N | 234 |
| R403E/E410N | | |
| A103N/N105S/Y155F/R318Y/R338E/ | A[103]N/N[105]S/Y[155]F/ | 235 |
| R403E/E410N | R150Y/R170E/R233E/E240N | |
| D104N/K106S/Y155F/R318Y/R338E | D[104]N/K[106]S/Y[155]F/ | 236 |
| /R403E/E410N | R150Y/R170E/R233E/E240N | |
| Y155F/K228N/R318Y/R338E/R403E/ | Y[155]F/K63N/R150Y/R170E/R233E/E240N | 237 |
| E410N | | |
| Y155F/I251S/R318Y/R338E/ | Y[155]F/I86S/R150Y/R170E/R233E/E240N | 238 |
| R403E/E410N | | |
| Y155F/K247N/N249S/R318Y/R338E/ | Y[155]F/K82N/N84S/R150Y/R170E/R233E/ | 239 |
| R403E/E410N | E240N | |
| K247N/N249S/R318Y/R338E/R403E/ | K82N/N84S/R150Y/R170E/R233E/E240N | 240 |
| E410N | | |
| Y155F/R318Y/R338E/R403E/E410N | Y[155]F/R150Y/R170E/R233E/E240N | 241 |
| K247N/N249S/R318Y/R338E/E410N | K82N/N84S/R150Y/R170E/E240N | 242 |
| Y155F/R318Y/R338E/E410N | Y[155]F/R150Y/R170E/E240N | 243 |
| Y155F/K247N/N249S/R318Y/R338E/ | Y[155]F/K82N/N84S/R150Y/R170E/E240N | 244 |
| E410N | | |
| D104N/K106S/Y155F/K228N/K247N/ | D[104]N/K[106]S/Y[155]F/ | 245 |
| N249S | K63N/K82N/N84S | |
| D104N/K106S/Y155F/K247N/N249S | D[104]N/K[106]S/Y[155]F/ | 246 |
| | K82N/N84S | |
| D104N/K106S/Y155F/K228N | D[104]N/K[106]S/Y[155]F/ | 247 |
| | K63N | |
| Y155F/K228N/K247N/N249S | Y[155]F/K63N/K82N/N84S | 248 |
| D104N/K106S/K228N/K247N/N249S | D[104]N/K[106]S/K63N/K82N/N84S | 184 |
| R318Y/R338E/R403E/E410S | R150Y/R170E/R233E/E240S | 249 |
| R318Y/R338E/R403E/E410N/T412V | R150Y/R170E/R233E/E240N/T242V | 250 |
| R318Y/R338E/R403E/E410N/T412A | R150Y/R170E/R233E/E240N/T242A | 251 |
| R318Y/R338E/R403E/T412A | R150Y/R170E/R233E/T242A | 252 |
| R318Y/R338E/E410S | R150Y/R170E/E240S | 253 |
| R318Y/R338E/T412A | R150Y/R170E/T242A | 254 |
| R318Y/R338E/E410N/T412V | R150Y/R170E/E240N/T242V | 255 |
| D85N/K228N/R318Y/R338E/R403E/ | D[85]N/K63N/R150Y/R170E/R233E/E240N | 256 |
| E410N | | |
| N260S/R318Y/R338E/R403E/E410N | N95S/R150Y/R170E/R233E/E240N | 257 |
| R318Y/R338E/N346D/R403E/E410N | R150Y/R170E/N178D/R233E/E240N | 258 |
| Y155F/N346D | Y[155]F/N178D | 259 |
| Y155F/R318Y/R338E/N346D/R403E/ | Y[155]F/R150Y/R170E/N178D/R233E/E240N | 260 |
| E410N | | |
| Y155F/N260S/N346D | Y[155]F/N95S/N178D | 261 |
| K247N/N249S/N260S | K82N/N84S/N95S | 262 |
| Y155F/N260S | Y[155]F/N95S | 263 |
| K247N/N249S/N260S/R318Y/R338E/ | K82N/N84S/N95S/R150Y/R170E/R233E/E240N | 264 |
| R403E/E410N | | |
| D104N/K106S/N260S/R318Y/R338E/ | D[104]N/K[106]S/N95S/R150Y/R170E/ | 265 |
| R403E/E410N | R233E/E240N | |
| Y155F/N260S/R318Y/R338E/R403E/ | Y[155]F/N95S/R150Y/R170E/R233E/E240N | 266 |
| E410N | | |
| R318Y/R338E/T343R/R403E/E410N | R150Y/R170E/T175R/R233E/E240N | 267 |
| R338E/T343R | R170E/T175R | 268 |
| D104N/K106S/Y155F/N260S | D[104]N/K[106]S/Y[155]F/N95S | 269 |
| Y155F/K247N/N249S/N260S | Y[155]F/K82N/N84S/N95S | 270 |
| D104N/K106S/K247N/N249S/N260S | D[104]N/K[106]S/K82N/N84S/N95S | 271 |
| D104N/K106S/Y155F/K247N/N249S/ | D[104]N/K[106]S/Y[155]F/K82N/ | 272 |
| N260S | N84S/N95S | |
| D104N/K106S/N260S | D[104]N/K[106]S/N95S | 185 |
| T343R/Y345T | T175R/Y177T | 215 |
| R318Y/R338E | R150Y/R170E | 188 |
| Y259F/K265T/Y345T | Y94F/K98T/Y177T | 216 |
| D104N/K106S/Y155F/K247N/N249S/ | D[104]N/K[106]S/Y[155]F/K82N/ | 326 |
| R318Y/R338E/R403E/E410N | N84S/R150Y/R170E/R233E/E240N | |
| D104N/K106S/K228N/K247N/N249S/ | D[104]N/K[106]S/K63N/K82N/N84S/R150Y/ | 327 |
| R318Y/R338E/R403E/E410N | R170E/R233E/E240N | |
| Y155F/K228N/K247N/N249S/R318Y/ | Y[155]F/K63N/K82N/N84S/R150Y/R170E/R233E/ | 328 |
| R338E/R403E/E410N | E240N | |
| Y155F/K247N/N249S/N260S/R318Y/ | Y[155]F/K82N/N84S/N95S/R150Y/R170E/R233E/ | 329 |
| R338E/R403E/E410N | E240N | |
| Y155F/R318Y/R338E/T343R/R403E/ | Y[155]F/R150Y/R170E/T175R/R233E/E240N | 330 |
| E410N | | |
| D104N/K106S/R318Y/R338E/T343R/ | D[104]N/K[106]S/R150Y/R170E/T175R/ | 331 |
| R403E/E410N | R233E/E240N | |
| T343R/N346Y | T175R/N178Y | 332 |
| R318Y/R338E/N346Y/R403E/E410N | R150Y/R170E/N178Y/R233E/E240N | 333 |
| R318Y/R338E/T343R/N346Y/R403E/ | R150Y/R170E/T175R/N178Y/R233E/E240N | 334 |
| E410N | | |
| T343R/N346D | T175R/N178D | 335 |
| R318Y/R338E/T343R/N346D/R403E/ | R150Y/R170E/T175R/N178D/R233E/E240N | 336 |
| E410N | | |
| R318Y/R338E/Y345A/R403E/E410N | R150Y/R170E/Y177A/R233E/E240N | 337 |
| R318Y/R338E/Y345A/N346D/R403E/ | R150Y/R170E/Y177A/N178D/R233E/E240N | 338 |
| E410N | | |
| Y155F/K247N/N249S/R318Y/R338E/ | Y[155]F/K82N/N84S/R150Y/R170E/R233E | 339 |
| R403E | | |
| K247N/N249S/R318Y/R338E/R403E | K82N/N84S/R150Y/R170E/R233E | 340 |
| Y155F/K247N/N249S/R318Y/R403E/ | Y[155]F/K82N/N84S/R150Y/R233E/E240N | 341 |
| E410N | | |
| K247N/N249S/R318Y/R403E/E410N | K82N/N84S/R150Y/R233E/E240N | 342 |
| Y155F/K247N/N249S/R338E/R403E/ | Y[155]F/K82N/N84S/R170E/R233E/E240N | 343 |
| E410N | | |
| K247N/N249S/R338E/R403E/E410N | K82N/N84S/R170E/R233E/E240N | 344 |
| R318Y/R338E/T343R/R403E | R150Y/R170E/T175R/R233E | 345 |
| Y155F/R318Y/R338E/T343R/R403E | Y[155]F/R150Y/R170E/T175R/R233E | 346 |
| R318Y/R338E/T343R/E410N | R150Y/R170E/T175R/E240N | 347 |
| Y155F/R318Y/R338E/T343R/E410N | Y[155]F/R150Y/R170E/T175R/E240N | 348 |
| R318Y/T343R/R403E/E410N | R150Y/T175R/R233E/E240N | 349 |
| Y155F/R318Y/T343R/R403E/E410N | Y[155]F/R150Y/T175R/R233E/E240N | 350 |
| R338E/T343R/R403E/E410N | R170E/T175R/R233E/E240N | 351 |
| Y155F/R338E/T343R/R403E/E410N | Y[155]F/R170E/T175R/R233E/E240N | 352 |
| Y155F/K247N/N249S/R318Y/R338E/ | Y[155]F/K82N/N84S/R150Y/R170E/T175R/ | 353 |
| T343R/R403E/E410N | R233E/E240N | |
| K247N/N249S/R318Y/R338E/T343R/ | K82N/N84S/R150Y/R170E/T175R/R233E/E240N | 354 |
| R403E/E410N | | |
| K228N/I251S/R318Y/R338E/R403E/ | K63N/I86S/R150Y/R170E/R233E/E240N | 355 |
| E410N | | |
| Y155F/K228N/I251S/R318Y/R338E/ | Y[155]F/K63N/I86S/R150Y/R170E/R233E/ | 356 |
| R403E/E410N | E240N | |
| N260S/R318Y/R338E/T343R/R403E/ | N95S/R150Y/R170E/T175R/R233E/E240N | 357 |
| E410N | | |
| Y155F/N260S/R318Y/R338E/T343R/ | Y[155]F/N95S/R150Y/R170E/T175R/R233E/ | 358 |
| R403E/E410N | E240N | |
| K228N/K247N/N249S/R318Y/R338E/ | K63N/K82N/N84S/R150Y/R170E/T175R/R233E/E240N | 359 |
| T343R/R403E/E410N | | |
| Y155F/K228N/K247N/N249S/R318Y/ | Y[155]F/K63N/K82N/N84S/R150Y/R170E/ | 360 |
| R338E/T343R/R403E/E410N | T175R/R233E/E240N | |
| Y155F/R338E/T343R/R403E | Y[155]F/R170E/T175R/R233E | 361 |
| R338E/T343R/R403E | R170E/T175R/R233E | 362 |
| Y155F/R338E/T343R/R403E/E410S | Y[155]F/R170E/T175R/R233E/E240S | 363 |
| Y155F/N260S/R338E/T343R/R403E | Y[155]F/N95S/R170E/T175R/R233E | 364 |
| Y155F/I251S/R338E/T343R/R403E | Y[155]F/I86S/R170E/T175R/R233E | 365 |
| R318Y/R338E/T343R/R403E/E410S | R150Y/R170E/T175R/R233E/E240S | 366 |
| Y155F/K247N/N249S/T343R/R403E | Y[155]F/K82N/N84S/T175R/R233E | 367 |
| Y155F/K247N/N249S/R318Y/R338E/ | Y[155]F/K82N/N84S/R150Y/R170E/T175R/ | 368 |
| T343R/R403E | R233E | |
| K247N/N249S/R318Y/R338E/T343R/ | K82N/N84S/R150Y/R170E/T175R/R233E | 369 |
| R403E | | |
| Y155F/K247N/N249S/R338E/T343R/ | Y[155]F/K82N/N84S/R170E/T175R/R233E/ | 370 |
| R403E/E410N | E240N | |
| K247N/N249S/R338E/T343R/R403E/ | K82N/N84S/R170E/T175R/R233E/E240N | 371 |
| E410N | | |
| Y155F/K247N/N249S/R318Y/R338E | Y[155]F/K82N/N84S/R150Y/R170E | 372 |
| Y155F/K247N/N249S/R318Y/T343R | Y[155]F/K82N/N84S/R150Y/T175R | 373 |
| Y155F/K247N/N249S/R318Y/R403E | Y[155]F/K82N/N84S/R150Y/R233E | 374 |
| Y155F/K247N/N249S/R318Y/E410N | Y[155]F/K82N/N84S/R150Y/E240N | 375 |
| Y155F/K247N/N249S/R338E/R403E | Y[155]F/K82N/N84S/R170E/R233E | 376 |
| Y155F/K247N/N249S/R338E/T343R | Y[155]F/K82N/N84S/R170E/T175R | 377 |
| Y155F/K247N/N249S/R318Y/R338E/ | Y[155]F/K82N/N84S/R150Y/R170E/T175R/ | 378 |
| T343R/E410N | E240N | |
| K247N/N249S/R318Y/R338E/T343R/ | K82N/N84S/R150Y/R170E/T175R/E240N | 379 |
| E410N | | |
| Y155F/K247N/N249S/R318Y/T343R/ | Y[155]F/K82N/N84S/R150Y/T175R/R233E/ | 380 |
| R403E/E410N | E240N | |
| K247N/N249S/R318Y/T343R/R403E/ | K82N/N84S/R150Y/T175R/R233E/E240N | 381 |
| E410N | | |
| Y155F/K247N/N249S/R338E/E410N | Y[155]F/K82N/N84S/R170E/E240N | 382 |
| Y155F/K247N/N249S/R318Y/T343R/ | Y[155]F/K82N/N84S/R150Y/T175R/R233E | 383 |
| R403E | | |
| K247N/N249S/R318Y/T343R/R403E | K82N/N84S/R150Y/T175R/R233E | 384 |
| Y155F/K247N/N249S/R318Y/T343R/ | Y[155]F/K82N/N84S/R150Y/T175R/E240N | 385 |
| E410N | | |
| K247N/N249S/R318Y/T343R/E410N | K82N/N84S/R150Y/T175R/E240N | 386 |
| Y155F/K247N/N249S/R338E/T343R/ | Y[155]F/K82N/N84S/R170E/T175R/R233E | 387 |
| R403E | | |
| K247N/N249S/R338E/T343R/R403E | K82N/N84S/R170E/T175R/R233E | 388 |
| Y155F/K247N/N249S/R338E/T343R/ | Y[155]F/K82N/N84S/R170E/T175R/E240N | 389 |
| E410N | | |
| K247N/N249S/R338E/T343R/E410N | K82N/N84S/R170E/T175R/E240N | 390 |
| Y155F/K247N/N249S/T343R/R403E/ | Y[155]F/K82N/N84S/T175R/R233E/E240N | 391 |
| E410N | | |
| K247N/N249S/T343R/R403E/E410N | K82N/N84S/T175R/R233E/E240N | 392 |
| Y155F/R318Y/R338E/T343R | Y[155]F/R150Y/R170E/T175R | 393 |
| R318Y/R338E/T343R | R150Y/R170E/T175R | 394 |
| Y155F/R318Y/T343R/R403E | Y[155]F/R150Y/T175R/R233E | 395 |
| Y155F/T343R/R403E/E410N | Y[155]F/T175R/R233E/E240N | 396 |
| Y155F/K247N/N249S/R318Y/R338E/ | Y[155]F/K82N/N84S/R150Y/R170E/T175R | 397 |
| T343R | | |
| K247N/N249S/R318Y/R338E/T343R | K82N/N84S/R150Y/R170E/T175R | 398 |
| Y155F/K247N/N249S/T343R/E410N | Y[155]F/K82N/N84S/T175R/E240N | 399 |
| Y155F/K247N/N249S/R403E/E410N | Y[155]F/K82N/N84S/R233E/E240N | 400 |
| Y155F/R338E/T343R/E410N | Y[155]F/R170E/T175R/E240N | 401 |
| R338E/T343R/E410N | R170E/T175R/E240N | 402 |
| Y155F/R318Y/T343R/E410N | Y[155]F/R150Y/T175R/E240N | 403 |
| R318Y/T343R/E410N | R150Y/T175R/E240N | 404 |
| K228N/R318Y/R338E/T343R/R403E/ | K63N/R150Y/R170E/T175R/R233E/E240N | 405 |
| E410N | | |
| K228N/K247N/N249S/R318Y/R338E/ | K63N/K82N/N84S/R150Y/R170E/T175R/R233E | 406 |
| T343R/R403E | | |
| K228N/K247N/N249S/R318Y/R338E/ | K63N/K82N/N84S/R150Y/R170E/T175R/E240N | 407 |
| T343R/E410N | | |
| K228N/K247N/N249S/R318Y/T343R/ | K63N/K82N/N84S/R150Y/T175R/R233E/E240N | 408 |
| R403E/E410N | | |
| Y155F/R338E/R403E/E410N | Y[155]F/R170E/R233E/E240N | 409 |
| Y155F/R318Y/R338E/R403E | Y[155]F/R150Y/R170E/R233E | 410 |
| Y155F/R318Y/R403E/E410N | Y[155]F/R150Y/R233E/E240N | 411 |

3. Conjugates and Fusion Proteins

The modified FIX polypeptides provided herein can be conjugated or fused to another polypeptide or other moiety, such as a polymer. In some instances, the conjugation or fusion is effected to increase serum half-life. Exemplary polypeptides to which the modified FIX polypeptides provided herein can be fused include, but are not limited to, serum albumin, Fc, FcRn, and transferrin (see, e.g., Sheffield, W. P. et al., (2004) Br. J. Haematol. 126(4):565-73; U.S. Patent Publication No. 2005/0147618; and International Application Publication Nos. WO 2007/112005 and WO 2004/101740).

The modified FIX polypeptides provided herein can be conjugated to a polymer, such as dextran, a polyethylene glycol (PEG) or sialyl moiety, or other such polymers, such as natural or sugar polymers. In one example, the polypeptides are conjugated to dextrans, such as described elsewhere (Zambaux et al., (1998) J. Protein Chem. 17(3):279-84). Various methods of modifying polypeptides by covalently attaching (conjugating) a PEG or PEG derivative (i.e., “PEGylation”) are known in the art (see e.g., U.S. 2006/0104968, U.S. Pat. Nos. 5,672,662, 6,737,505 and U.S. 2004/0235734). Techniques for PEGylation include, but are not limited to, specialized linkers and coupling chemistries (see, e.g., Harris, (2002) Adv. Drug Deliv. Rev. 54:459-476), attachment of multiple PEG moieties to a single conjugation site (such as via use of branched PEGs; see, e.g., Veronese et al., (2002) Bioorg. Med. Chem. Lett. 12:177-180), site-specific PEGylation and/or mono-PEGylation (see, e.g., Chapman et al., (1999) Nature Biotech. 17:780-783), site-directed enzymatic PEGylation (see, e.g., Sato, (2002) Adv. Drug Deliv. Rev., 54:487-504, 2002), and glycoPEGylation (see, e.g., U.S. Patent Publication Nos. 2008/0050772, 2008/0146494, 2008/0050772, 2008/0187955, and 2008/0206808). Methods and techniques described in the art can produce proteins having 1, 2, 3, 4, 5, 6, 7, 8, 9, 10 or more PEG or PEG derivatives attached to a single protein molecule (see, e.g., U.S. 2006/0104968). Thus, the modified FIX polypeptide provided herein can be PEGylated, including glycoPEGylated, using any method known in the art, such as any described in U.S. Pat. Nos. 5,969,040, 5,621,039, 6,423,826; U.S. Patent Publication Nos. 2003/0211094, 2007/0254840, 2008/0188414, 2008/000422, 2008/0050772, 2008/0146494, 2008/0050772, 2008/0187955 and 2008/0206808; and International Application Publication Nos. WO 2007/112005, WO 2007/135182, WO 2008/082613, WO 2008/119815, and WO 2008/119815.

In some instances, the modified FIX polypeptides include amino acid replacements to facilitate conjugation to another moiety. For example, cysteine residues can be incorporated into the FIX polypeptide to facilitate conjugation to polymers. Exemplary amino acid replacement modifications for this purpose include, but are not limited to, D47C, Q50C, S53C, L57C, I66C, N67C, S68C, E70C, W72C, P74C, K80C, L84C, V86C, N89C, I90C, K91C, R94C, K100C, N101C, S102C, A103C, D104C, N105C, K106C, V108C, E114C, R116C, E119C, N120C, Q121C, S123C, E125C, P129C, S138C, T140C, S141C, K142C, A146C, E147C, E162C, T163C, I164C, L165C, D166C, N167C, I168C, T169C, Q170C, S171C, T172C, Q173C, S174C, F175C, N176C, D177C, F178C, T179C, R180C, E185C, D186C, K188C, P189C, K201C, V202C, D203C, E224C, T225C, K228C, E239C, E240C, T241C, H243C, K247C, N249C, R252C, H257C, N260C, A261C, A262C, I263C, K265C, E277C, F314C, R318C, L321C, K341C, E372C, E374C, M391C, K392C, N406C, K413C, and T415C (corresponding to a mature FIX polypeptide set forth in SEQ ID NO:3).

### F. PRODUCTION OF FIX POLYPEPTIDES

FIX polypeptides, including modified FIX polypeptides, or domains thereof, of FIX can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a FIX polypeptide or other vitamin-K polypeptide, such as from a cell or tissue source, such as for example from liver. Modified FIX polypeptides can be engineered as described herein, such as by site-directed mutagenesis.

FIX can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a FIX polypeptide, including for example, polymerase chain reaction (PCR) methods. A nucleic acid containing material can be used as a starting material from which a FIX-encoding nucleic acid molecule can be isolated. For example, DNA and mRNA preparations, cell extracts, tissue extracts (e.g., from liver), fluid samples (e.g., blood, serum, saliva), samples from healthy and/or diseased subjects can be used in amplification methods. Nucleic acid libraries also can be used as a source of starting material. Primers can be designed to amplify a FIX-encoding molecule. For example, primers can be designed based on expressed sequences from which a FIX is generated. Primers can be designed based on back-translation of a FIX amino acid sequence. Nucleic acid molecules generated by amplification can be sequenced and confirmed to encode a FIX polypeptide.

Additional nucleotide sequences can be joined to a FIX-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a FIX-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to FIX-encoding nucleic acid molecules. Such regions include, but are not limited to, sequences to facilitate uptake of FIX into specific target cells, or otherwise enhance the pharmacokinetics of the synthetic gene.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the Bluescript vector (Stratagene, La Jolla, Calif.). The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and FIX protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated FIX protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA. Details of cloning and expression of the modified FIX polypeptides are described in U.S. Pat. Nos. 9,328,339 and 8,778,870.

Nucleic acid molecules encoding FIX or modified FIX polypeptides are provided herein. Nucleic acid molecules include allelic variants or splice variants of any encoded FIX polypeptide. Exemplary of nucleic acid molecules provided herein are any that encode a modified FIX polypeptide provided herein, such as any encoding a polypeptide set forth in any of SEQ ID NOs:75-272, and those including introns as described herein and exemplified in the Sequence Listing.

In some embodiments, nucleic acid molecules provided herein are optimized for expression in a mammal, such as a human or a mouse. It is found that mouse optimized codons are at least about 90% identical to human optimized codons. The nucleic acid molecules provided herein have at least 50, 60, 65, 70, 75, 80, 85, 90, 91, 92, 93, 94, 95, or 99% sequence identity or hybridize under conditions of medium or high stringency along at least 70% of the full-length of any nucleic acid encoding a FIX polypeptide provided herein. For example, the nucleic acid molecules provided herein have at least or at least about 50, 60, 65, 70, 75, 80, 85, 90, 91, 92, 93, 94, 95, or 99% sequence identity to the nucleic acid sequence set forth in SEQ ID NO:1. In another embodiment, a nucleic acid molecule can include those with degenerate codon sequences, including the optimized sequences encoding any of the FIX polypeptides provided herein.

### G. ASSESSING MODIFIED FIX POLYPEPTIDE ACTIVITIES

The activities and properties of FIX polypeptides can be assessed in vitro and/or in vivo. Assays for such assessment are known to those of skill in the art and are known to correlate tested activities and results to therapeutic and in vivo activities. In one example, FIX variants can be assessed in comparison to unmodified and/or wild-type FIX. Such assays can be performed in the presence or absence of FVIIIa, phospholipids and/or calcium. In vitro assays include any laboratory assay known to one of skill in the art, such as for example, cell-based assays including coagulation assays, binding assays, protein assays, and molecular biology assays. In vivo assays include FIX assays in animal models as well as administration to humans. In some cases, activity of FIX polypeptides in vivo can be determined by assessing blood, serum, or other bodily fluid for assay determinants. FIX variants, such as those provided herein, also can be tested in vivo to assess an activity or property, such as therapeutic effect.

Typically, assays described herein are with respect to the two-chain activated form of FIX, i.e., FIXa. FIX polypeptides that have been activated via proteolytic cleavage after R145 and R180 can be prepared in vitro. The FIX polypeptides can be first prepared by any of the methods of production described herein, including, but not limited to, production in mammalian cells followed by purification. Cleavage of the FIX polypeptides into the active protease form of FIX can be accomplished by incubation with activated Factor XI (FXIa). The activated polypeptides can be used in any of the assays to measure FIX activities described herein. Such assays also can be performed with the single chain zymogen form. For example, a single chain zymogen FIX polypeptide can provide a negative control since such a form typically does not exhibit the proteolytic or catalytic activity required for the coagulant activity of FIX. In addition, such assays also can be performed in the presence of cofactors, such as FVIIIa, and other molecules, such as phospholipids and/or calcium, which in can augment the activity of FIX.

1. In Vitro Assays

Exemplary in vitro assays include assays to assess polypeptide modification and activity. Modifications can be assessed using in vitro assays that assess glycosylation, γ-carboxylation and other post-translational modifications, protein assays and conformational assays known in the art. Assays for activity include, but are not limited to, measurement of FIX interaction with other coagulation factors, such as FVIIIa and Factor X, proteolytic assays to determine the proteolytic activity of FIX polypeptides, assays to determine the binding and/or affinity of FIX polypeptides for phosphatidylserines and other phospholipids, and cell based assays to determine the effect of FIX polypeptides on coagulation.

Concentrations of modified FIX polypeptides can be assessed by methods well-known in the art, including but not limited to, enzyme-linked immunosorbent assays (ELISA), SDS-PAGE; Bradford, Lowry, BCA methods; UV absorbance, and other quantifiable protein labeling methods, such as, but not limited to, immunological, radioactive and fluorescent methods and related methods. Assessment of cleavage products of proteolysis reactions, including cleavage of FIX polypeptides or products produced by FIX protease activity, can be performed using methods including, but not limited to, chromogenic substrate cleavage, HPLC, SDS-PAGE analysis, ELISA, Western blotting, immunohistochemistry, immunoprecipitation, NH₂-terminal sequencing, fluorescence, and protein labeling.

Structural properties of modified FIX polypeptides can also be assessed. For example, X-ray crystallography, nuclear magnetic resonance (NMR), and cryoelectron microscopy (cryo-EM) of modified FIX polypeptides can be performed to assess three-dimensional structure of the FIX polypeptides and/or other properties of FIX polypeptides, such as Ca′ or cofactor binding.

Additionally, the presence and extent of FIX degradation can be measured by standard techniques such as sodium dodecyl sulfate polyacrylamide gel electrophoresis (SDS-PAGE), and Western blotting of electrophoresed FIX-containing samples. FIX polypeptides that have been exposed to proteases also can be subjected to N-terminal sequencing to determine location or changes in cleavage sites of the modified FIX polypeptides.

a. Glycosylation

FIX polypeptides can be assessed for the presence of glycosylation using methods well known in the art. Glycosylation of a polypeptide can been characterized from its enzymatically or chemically released carbohydrate pool, using a wide variety of methods, such as high pH anion exchange chromatography (Townsend et al., (1991) Glycobiology 1:139-147), or fluorophore-assisted carbohydrate electrophoresis (FACE) (Kumar et al., (1996) Biotechnol. Appl. Biochem. 24:207-214.), sequential exoglycosidase digestions (Watzlawick et al., (1992) Biochemistry 31:12198-12203; Tyagaraj an et al., (1996) Glycobiology, 6:83-93), mass spectrometry (Gillece-Castro et al., (1990) Meth. Enzymol. 193: 689-712; Duffin et al., (1992) Anal. Chem. 64:1440-1448; Papac et al., (1997) in Techniques in Glycobiology (Townsend R. R. and Hotchkiss A. T. eds.) Marcel Decker, Inc., New York, pp. 33-52; Fu et al., (1994) Carbohydr. Res. 261:173-186), and NMR (Fu et al., (1994) Carbohydr. Res. 261:173-186).

For example, chemical release can be effected by hydrazinolysis, which releases N- and O-linked glycans from glycoproteins by incubation with anhydrous hydrazine. Enzymatic release can be effected by the endoglycosidases peptide N-glycosidase F (PNGase F), which removes unaltered most of the common N-linked carbohydrates from the polypeptide while hydrolyzing the originally glycosylated Asn residue to Asp. Hydrazinolysis or endoglycosidase treatment of FIX polypeptides generates a reducing terminus that can be tagged with a fluorophore or chromophore label. Labeled FIX polypeptides can be analyzed by fluorophore-assisted carbohydrate electrophoresis (FACE). The fluorescent tag for glycans also can be used for monosaccharide analysis, profiling or fingerprinting of complex glycosylation patterns by HPLC. Exemplary HPLC methods include hydrophilic interaction chromatography, electronic interaction, ion-exchange, hydrophobic interaction, and size-exclusion chromatography. Exemplary glycan probes include, but are not limited to, 3-(acetylamino)-6-aminoacridine (AA-Ac) and 2-aminobenzoic acid (2-AA). Carbohydrate moieties can also be detected through use of specific antibodies that recognize the glycosylated FIX polypeptide.

In one method, mass spectrometry is used to assess site-specific carbohydrate heterogeneity. This can involve matrix-assisted laser desorption ionization mass spectrometry of collected HPLC-fractions (Sutton et al., (1994) Anal. Biochem. 218:34-46; Ploug et al., (1998) J. Biol. Chem. 273:13933-13943), or reversed phase HPLC directly coupled with electrospray ionization mass spectrometry (LC/ESIMS) (see, e.g., Huddleston et al., (1993) Anal. Chem. 65:877-884; Medzihradsky et al., (2008) Methods Mol. Biol. 446:293-316). In one example, glycosylation at potential N-glycosylation sites, such as an asparagine residue within an Asn-X-Ser/Thr/Cys motif, is assessed by LC/ESIMS. The potential N-glycosylation sites in a FIX polypeptide can be identified, and a proteolytic enzyme can be selected that would separate these sites on individual peptides. The digestion mixture is then analyzed by LC/ESIMS, a method that generates diagnostic carbohydrate ions by collisional activation (33). These diagnostic carbohydrate ions include, for example, characteristic non-reducing end oxonium ions at m/z 204, 274 and 292, 366, and 657, which indicate the presence of N-acetylhexosamine, neuraminic (sialic) acid, hexosyl-N-acetylhexosamine, and sialyl-hexosyl-Nacetylhexosamine, respectively. In addition to identifying the presence of these ions by selective ion monitoring (SIM), the LC/ESIMS method also analyzes the peptide to assess the molecular weight, which can be used to indicate which peptide, and, therefore, which potential N-glycosylation site, contains the carbohydrate.

b. Other Post-Translational Modifications

FIX polypeptides can be assessed for the presence of post-translational modifications other than glycosylation. Such assays are known in the art and include assays to measure hydroxylation, sulfation, phosphorylation and carboxylation. An exemplary assay to measure β-hydroxylation comprises reverse phase HPLC analysis of FIX polypeptides that have been subjected to alkaline hydrolysis (Przysiecki et al. (1987) Proc. Natl. Acad. Sci. U.S.A. 84:7856-7860). Carboxylation and γ-carboxylation of FIX polypeptides can be assessed using mass spectrometry with matrix-assisted laser desorption ionization time-of-flight (MALDI-TOF) analysis, as described for other vitamin K-dependent polypeptides (see, e.g., Harvey et al. (2003) J. Biol. Chem. 278:8363-8369; Maun et al. (2005) Prot. Sci. 14:1171-1180). The interaction of a FIX polypeptide containing the propeptide (pro-FIX) with the carboxylase responsible for post-translational γ-carboxylate modification also can be assessed. The dissociation constant (K_{d}) following incubation of carboxylase with fluorescein-labeled pro-FIX polypeptides can be measured by determining the amount of bound carboxylase by anisotropy (Lin et al. (2004) J. Biol. Chem. 279:6560-6566). Other exemplary assays to measure carboxylation include reverse phase HPLC analysis of FIX polypeptides that have been subjected to alkaline hydrolysis (Przysiecki et al. (1987) Proc. Natl. Acad. Sci. U.S.A. 84:7856-7860).

Exemplary assays to measure phosphorylation include use of phosphospecific antibodies to phosphoserine and/or -tyrosine amino acid residues or to a serine-phosphorylated FIX polypeptide. ³²P metabolic labeling of cells that produce the FIX polypeptide also can be used to assess phosphorylation, wherein the labeled FIX polypeptide can be purified and analyzed for incorporation of radioactive phosphate. An exemplary assay for tyrosine sulfation includes ³⁵S labeling of cells that produce the FIX polypeptide. In such method, cells are incubated with either ³⁵S—S₂SO₄ or ³⁵S-methionine and incorporation of the ³⁵S is determined by normalization to the ³⁵S-methionine sample.

c. Proteolytic Activity

Modified FIX polypeptides can be tested for proteolytic activity towards both synthetic substrates and its natural substrate, Factor X. Activated forms of the modified FIX polypeptides (FIXa) typically are used in in vitro assays. Assays using a synthetic substrate, such as a CH₃SO₂-LGR-pNA peptide, can be employed to measure enzymatic cleavage activity of the FIXa polypeptides. Hydrolysis of CH₃SO₂-LGR-pNA in the presence of FIXa can be measured by assessing the production of p-nitroanaline (pNA) from the cleavage reaction sample. The amount of pNA in the sample is proportional to the absorbance of the sample at 405 nm and thus indicates the extent of proteolytic activity in the FIXa sample. Additional exemplary fluorogenic substrates that can be used to assess FIXa cleavage activity include, but are not limited to, Mes-D-CHD-Gly-Arg-AMC (Pefafluor FIXa10148) and H-D-Leu-PHG-Arg-AMC (Pefafluor FIXa3688), wherein cleavage is assessed by release of AMC, and the fluorogenic ester substrate, 4-methylumbelliferyl p′-guanidinobenzoate (MUGB), where cleavage is assessed by the release of 4-methylumbelliferone fluorophore (4-MU) (see, e.g., Example 3). Molecules that enhance FIXa catalytic activity, such as ethylene glycol, can be employed in such assays (Sturzebecher et al. (1997) FEBS Lett. (412):295-300).

Proteolytic activity of FIXa also can be assessed by measuring the conversion of factor X (FX) into activated Factor X (FXa), such as described in Example 4, below. FIXa polypeptides, including the modified FIX polypeptides provided herein, can be incubated with FX polypeptides in the presence of FVIIIa, phospholipids vesicles (phosphatidylserine and/or phosphatidylcholine) and Ca²⁺, and cleavage of FX to produce FXa can be assayed using a fluorogenic substrate, such as Spectrafluor Fxa (CH₃SO₂-D-CHA-Gly-Arg-AMC), or a chromogenic substrate, such as S2222 or S2765 (Chromogenics AB, Molndal, Sweden), which are specifically cleaved by FXa.

d. Coagulation Activity

FIX polypeptides can be tested for coagulation activity by using assays well known in the art. For example, some of the assays include, but are not limited to, a two stage clotting assay (Liebman et al., (1985) Proc. Natl. Acad. Sci. U.S.A. 82:3879-3883); the prothrombin time assay (PT, which can measure TF-dependent activity of FIXa in the extrinsic pathway); assays which are modifications of the PT test; the activated partial thromboplastin time (aPTT, which can measure TF-independent activity of FIXa); activated clotting time (ACT); recalcified activated clotting time; the Lee-White Clotting time; or thromboelastography (TEG) (Pusateri et al. (2005) Critical Care 9:S15-S24). For example, coagulation activity of a modified FIX polypeptide can be determined by a PT-based assay where FIX is diluted in FIX-deficient plasma, and mixed with prothrombin time reagent (recombinant TF with phospholipids and calcium), such as that available as Innovin™ from Dade Behring. Clot formation is detected optically and time to clot is determined and compared against FIX-deficient plasma alone. In vivo coagulation assays in animal models, such as those described below, also can be performed to assess the coagulation activity of FIX polypeptides.

e. Binding to and/or Inhibition by Other Proteins and Molecules

Inhibition assays can be used to measure resistance of modified FIX polypeptides to FIX inhibitors, such as, for example, antithrombin III (AT-III), heparin, AT-III/heparin complex, p-aminobenzamidine, serine protease inhibitors, and FIX-specific antibodies. Assessment of inhibition to other inhibitors also can be tested and include, but are not limited to, other serine protease inhibitors. Inhibition can be assessed by incubation of the inhibitor with FIX polypeptides that have been pre-incubated with and/or without FVIIIa. The activity of FIX can then be measured using any one or more of the activity or coagulation assays described above, and inhibition by the inhibitor can be assessed by comparing the activity of FIX polypeptides incubated with the inhibitor, with the activity of FIX polypeptides that were not incubated with the inhibitor. For example, the inhibition of modified FIX polypeptides by AT-III/heparin can be assessed as described in Example 5, below. Inhibition of wild-type FIXa or FIXa variants by the AT-III/heparin complex is assessed by incubating AT-III/heparin with FIXa and the measuring the catalytic activity of FIXa towards a small molecule substrate, Mesyl-D-CHG-Gly-Arg-AMC (Pefafluor FIXa; Pentapharm). Such assays can be performed in the presence or absence of FVIIIa.

FIX polypeptides also can be tested for binding to other coagulation factors and inhibitors. For example, FIX direct and indirect interactions with cofactors, such as FVIIIa, substrates, such as FX and FIX, and inhibitors, such as antithrombin III and heparin, can be assessed using any binding assay known in the art, including, but not limited to, immunoprecipitation, column purification, non-reducing SDS-PAGE, BIAcore® assays, surface plasmon resonance (SPR), fluorescence resonance energy transfer (FRET), fluorescence polarization (FP), isothermal titration calorimetry (ITC), circular dichroism (CD), protein fragment complementation assays (PCA), Nuclear Magnetic Resonance (NMR) spectroscopy, light scattering, sedimentation equilibrium, small-zone gel filtration chromatography, gel retardation, Far-western blotting, fluorescence polarization, hydroxyl-radical protein foot printing, phage display, and various two-hybrid systems.

f. Phospholipid Affinity

Modified FIX polypeptide binding and/or affinity for phosphatidylserine (PS) and other phospholipids can be determined using assays well known in the art. Highly pure phospholipids (for example, known concentrations of bovine PS and egg phosphatidylcholine (PC), which are commercially available, such as from Sigma, in organic solvent can be used to prepare small unilamellar phospholipid vesicles. FIX polypeptide binding to these PS/PC vesicles can be determined by relative light scattering at 90° to the incident light. The intensity of the light scatter with PC/PS alone and with PC/PS/FIX is measured to determine the dissociation constant (Harvey et al., (2003) J. Biol. Chem. 278:8363-8369). Surface plasma resonance, such as on a BIAcore biosensor instrument, also can be used to measure the affinity of FIX polypeptides for phospholipid membranes (Sun et al., (2003) Blood 101:2277-2284).

2. Non-Human Animal Models

Non-human animal models can be used to assess activity and stability of modified FIX polypeptides. For example, non-human animals can be used as models for a disease or condition. Non-human animals can be injected with disease and/or phenotype-inducing substances prior to administration of FIX variants to monitor the effects on disease progression. Genetic models also are useful. Animals, such as mice, can be generated which mimic a disease or condition by the overexpression, under-expression, or knock-out of one or more genes. Such animals can be generated by transgenic animal production techniques well-known in the art or using naturally-occurring or induced mutant strains. Examples of useful non-human animal models of diseases associated with FIX include, but are not limited to, models of bleeding disorders, in particular hemophilia. These non-human animal models can be used to monitor activity of FIX variants compared to a wild type FIX polypeptide.

Animal models also can be used to monitor stability, half-life, clearance, and other pharmacokinetic and pharmacodynamic properties of modified FIX polypeptides. Such assays are useful for comparing modified FIX polypeptides and for calculating doses and dose regimens for further non-human animal and human trials. For example, a modified FIX polypeptide can be injected into the tail vein of mice. Blood samples are then taken at time-points after injection (such as minutes, hours and days afterwards) and then the pharmacokinetic and pharmacodynamic properties of the modified FIX polypeptides assessed, such as by monitoring the serum or plasma at specific time-points for FIXa activity and protein concentration by ELISA or radioimmunoassay (see, e.g., Example 6). Blood samples also can be tested for coagulation activity in methods, such as the aPTT assay (see, e.g., Example 6).

Modified FIX polypeptides can be tested for therapeutic effectiveness using animal models for hemophilia. In one non-limiting example, an animal model such as a mouse can be used. Mouse models of hemophilia are available in the art and include FIX deficient mice (such as those utilized in Example 7, below) and mice expressing mutant FIX polypeptides, and can be employed to test modified FIX polypeptides (Wang et al., (1997) Proc. Natl. Acad. Sci. U.S.A. 94:11563-11566; Lin et al., (1997) Blood 90:3962-3966; Kundu et al., (1998) Blood 92: 168-174; Sabatino et al., (2004) Blood 104(9):2767-2774; and Jin et al., (2004) Blood 104:1733-1739; see also Example 7).

Other models of FIX deficiencies include hemophilic dogs that express defective FIX or that have been hepatectomized (Evans et al., (1989) Proc. Natl. Acad. Sci. U.S.A. 86:10095; Mauser et al., (1996) Blood 88:3451; and Kay et al., (1994) Proc. Natl. Acad. Sci. U.S.A. 91:2353-2357).

3. Clinical Assays

Many assays are available to assess activity of FIX for clinical use. Such assays can include assessment of coagulation, protein stability, and half-life in vivo and phenotypic assays. Phenotypic assays and assays to assess the therapeutic effect of FIX treatment include assessment of blood levels of FIX (such as measurement of serum FIX prior to administration and time-points following administrations including, after the first administration, immediately after last administration, and time-points in between, correcting for the body mass index (BMI)), phenotypic response to FIX treatment including amelioration of symptoms over time compared to subjects treated with an unmodified and/or wild type FIX or placebo. Examples of clinical assays to assess FIX activity can be found, for example, in Franchini et al., (2005) J. Thromb. Haemost. 93(6):1027-1035; Shapiro et al., (2005) Blood 105(2):518-525; and White et al., (1997) J. Thromb. Haemost. 78(1):261-265. Patients can be monitored regularly over a period of time for routine or repeated administrations, following administration in response to acute events, such as hemorrhage, trauma, or surgical procedures.

### H. FORMULATION AND ADMINISTRATION

Compositions for use for gene therapy that contain the AAV vectors described herein for the treatment of bleeding disorders are provided. Such compositions contain a therapeutically effective amount of an AAV vector as described herein. Effective concentrations for administration for gene therapy are mixed with a suitable pharmaceutical carrier or vehicle for systemic, topical or local administration. Generally the gene therapy vectors are administered intravenously or by direct injection into the liver, or by direct injection into a compartmentalized liver (see, U.S. Pat. No. 9,821,114, for a description of methods of direct injection into a compartmentalized liver). Because of the properties of the vectors and encoded FIX effective dosages are at least 1 to 2 orders of magnitude lower, and, can be as much as 3 or 4 orders of magnitude lower, than prior AAV vectors encoding FIX for gene therapy to treat hemophilia.

Pharmaceutical carriers or vehicles suitable for administration of the compounds provided herein include any such carriers known to those skilled in the art to be suitable for the particular mode of administration. Pharmaceutical compositions that include a therapeutically effective amount of a FIX polypeptide described herein also can be provided as a lyophilized powder that is reconstituted, such as with sterile water, immediately prior to administration.

1. Formulations

The pharmaceutical compositions containing the vectors can be formulated in any conventional manner by mixing a selected amount of the vector and one or more physiologically acceptable carriers or excipients. Selection of the carrier or excipient is within the skill of the administering profession and can depend upon a number of parameters. These include, for example, the mode of administration (i.e., systemic, oral, nasal, pulmonary, local, topical, or any other mode) and disorder treated. The pharmaceutical compositions provided herein can be formulated for single dosage (direct) administration or for dilution or other modification. The concentrations of the vectors in the formulations are effective for delivery of an amount, upon administration, that is effective for the intended treatment. To formulate a composition, the weight fraction of the vector is dissolved, suspended, dispersed, or otherwise mixed in a selected vehicle at an effective concentration such that the treated condition is relieved or ameliorated.

The formulation should suit the mode of administration. For example, the vectors can be formulated for parenteral administration by injection (e.g., by bolus injection or continuous infusion). The injectable compositions can take such forms as suspensions, solutions or emulsions in oily or aqueous vehicles. The sterile injectable preparation also can be a sterile injectable solution or suspension in a non-toxic parenterally-acceptable diluent or solvent, for example, as a solution in 1,4-butanediol. Sterile, fixed oils are conventionally employed as a solvent or suspending medium. For this purpose any bland fixed oil can be employed, including, but not limited to, synthetic mono- or diglycerides, fatty acids (including oleic acid), naturally occurring vegetable oils like sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation.

The vectors can be formulated as the sole pharmaceutically active ingredient in the composition or can be combined with other active ingredients. The vectors can be provided in liposomes. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices such as collagen gels and liposomes modified with fibronectin (see, for example, Weiner et al., (1985) J. Pharm. Sci. 74(9):922-925). The compositions provided herein further can contain one or more adjuvants that facilitate delivery, such as, but are not limited to, inert carriers, or colloidal dispersion systems. Representative and non-limiting examples of such inert carriers can be selected from water, isopropyl alcohol, gaseous fluorocarbons, ethyl alcohol, polyvinyl pyrrolidone, propylene glycol, a gel-producing material, stearyl alcohol, stearic acid, spermaceti, sorbitan monooleate, methylcellulose, as well as suitable combinations of two or more thereof.

The amount of vector included in the pharmaceutically acceptable carrier in an amount sufficient to exert a therapeutically useful effect in the absence of undesirable side effects, such as immune reactions, on the subject treated. The therapeutically effective concentration can be determined empirically by testing in known in vitro and in vivo systems, such as the assays provided herein.

2. Dosages

The precise amount or dose of the therapeutic agent administered depends on the particular encoded FIX polypeptide, the route of administration, and other considerations, such as the severity of the disease and the weight and general state of the subject. Local administration of the therapeutic agent will typically require a smaller dosage than any mode of systemic administration, although the local concentration of the therapeutic agent can, in some cases, be higher following local administration than can be achieved with safety upon systemic administration. If necessary, a particular dosage and duration and treatment protocol can be empirically determined or extrapolated. Treatment by gene therapy, generally, is a single dose or several doses spaced over a period of time. The gene therapy can last for years, but can be repeated if the levels of the encoded FIX decrease. Dosage also is function of the severity of the factor IX deficiency, and the particular subject. For example, patients with severe Hemophilia B (FIX activity of <1 IU/dL; 1% of normal activity (where 1 IU represents the activity of Factor IX in 1 mL of normal, pooled plasma)) will require more FIX than patients with moderate (FIX activity of 1-5 IU/dL; 1-5% of normal activity), or mild (FIX activity of >5-<40 IU/dL; >5-<40% of normal activity) hemophilia B. For comparison, the initial estimated dose of BeneFIX® Factor IX can be determined using the following formula: Required units=body weight (kg)×desired factor IX increase (IU/dL or % of normal)×reciprocal of observed recovery (IU/kg per IU/dL). In clinical studies with adult and pediatric (<15 years) patients, one IU of BeneFIX® FIX per kilogram of body weight increased the circulating activity of Factor IX as follows: Adults: 0.8±0.2 IU/dL [range 0.4 to 1.2 IU/dL]; Pediatric: 0.7±0.3 IU/dL [range 0.2 to 2.1 IU/dL]. Thus, for adult patients: the number of Factor IX IU required (IU)=body weight (kg) x desired factor IX increase (% or IU/dL)×1.3 (IU/kg per IU/dL), and, for pediatric patients: the number of Factor IX IU required (IU)=body weight (kg) x desired factor IX increase (% or IU/dL)×1.4 (IU/kg per IU/dL).

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, the severity of the hemophilia, and other treatments or compounds, being administered concurrently. In addition to the above factors, such levels can be affected by the transduction and tropism potential of the AAV, and composition of the AAV vector, including the composition of the cassette flanked by the ITRs (e.g., promoter, enhancer or ITR composition), as can be determined by one skilled in the art.

Clinical trials of AAV-FIX have been conducted. In a completed trial, subjects were dosed with a single dose of AAV-FIX encoding hFIX (DTX101) at 1.6 E+12 or 5 E+12 genome copies/kg (gc/kg) via intravenous infusion (ClinicalTrials.gov Identifier: NCT02618915). Another completed clinical trial (ClinicalTrials.gov Identifier: NCT 02484092; Spark Therapeutics and Pfizer) in which patients were infused with AAV encoding FIX R338L (Padua) under control of a liver specific promoter at 5 E+11 vg/kg showed increased FIX coagulant activity (mean steady state activity of 33.7±18.5%). After 492 weeks, the annualized bleeding rate was significantly reduced compared to before vector administration (see, George et al. (2017) New Eng. J. Med. 377:2215-2227). In another clinical study, fifteen adult hemophilia B patients were infused with 5 E+11 vg/kg of fidanacogene elaparvovec (Pfizer/Spark Therapeutics). The results show that at one year post vector infusion, the mean post-infusion steady-state of FIX was 22.9%±9.9%. Mean ABR during the first 52 weeks following fidanacogene elaparvovec infusion was 0.4±1.1 compared to 8.9±14.0 in the 52 weeks preceding infusion (p<0.001). No bleeds were reported in 80% of patients, and all patients reported reduced bleeding frequency and decreased FIX use for the 52 weeks post-vector infusion. All patients reported no serious adverse events (Dec. 8, 2019: Presented at ASH annual Meeting Poster 3347).

Other clinical trials for treatment of hemophilia with AAV-FIX were ongoing in 2020 (ClinicalTrials.gov Identifiers: NCT 03185897; NCT 03861273 and NCT03587116 (both rAAV Spark100 hFIX Padua)). For example, subjects are dosed with a single dose of AAV-FIX (BBM-H901) at 5 E+12 vg/kg via intravenous infusion (ClinicalTrials.gov Identifier: NCT04135300). In another example, subjects are dosed with a single dose of AAV5-Padua FIX under control of a liver specific promoter (AMT-061) at 2 E+13 genome copies/kg (gc/kg) (i.e., vector genomes/kg) via intravenous infusion (ClinicalTrials.gov Identifier: NCT03489291).

In the methods herein, appropriate minimum dosage levels and dosage regimes of viruses, such as an AAV vector packaged in a capsid, such as the AAV vector and capsid described herein, can be levels sufficient for AAV delivery to the target site (e.g., the liver) and for the AAV to transduce the target tissue, such as the liver (e.g., hepatocytes). The dosages using the AAV vectors provided and described herein should be at least 1 order of magnitude lower than those in the previous clinical studies, such as those discussed above.

Generally, the capsid packaged AAV for expressing FIX (e.g., the modified FIX polypeptide set forth in SEQ ID NO: 394 or others of the modified FIX polypeptides provided herein) is administered in an amount that is at least or about or 1 E+10 vector genomes/kg of body weigh at least one time over a cycle of administration. Lower doses also are contemplated; particular doses are within the skill of the skilled practitioner. Factors include the severity of the hemophilia. Exemplary minimum levels for administering a virus to a 75 kg human can include at least about 1×10¹¹, or at least 1×10¹¹ vector genomes (vg), at least about 5×10¹¹ vg, at least about 1×10¹² vg, at least about 5×10¹² vg, at least about 1×10¹³ vg, at least about 5×10¹³ vg, at least about 1×10¹⁴ vg, or at least about 5×10¹⁴ vg. For example, the virus is administered in an amount that is at least or about or is 1×10¹¹ vg, 1×10¹² vg, 1×10¹³ vg, or 1×10¹⁴ vg, at least one time over a cycle of administration. For some subjects, a single dosage is sufficient for expression to be sustained for at least a year, generally longer.

For the vectors provided herein, dosages can be at least one order of magnitude lower than prior art doses. Reported AAV gene therapy doses include 2 e+13, 5 e+11, 2 e+12, and 4.5 e+11 vg/kg or gc/kg. The vectors herein can be dosed as low as 1 e+8 depending upon the route of administration. For example, as shown in the Examples below in the mouse models, compared to the TAK vector, the exemplified vectors herein, dosed at 8 e+10 vg/kg dose in mice achieved results comparable to the TAK vector, which was dosed at 7.4 e+11 vg/kg resulting in ˜20 U/mL. Thus, the vectors herein provide at least 10-fold more FIX, and can be dosed at least 1/10 dose. As described herein, the target for treatment is to result at least mild hemophilia, in which the activity of FIX is 20-50%, such as at least 30% or at least 40%, FIX, up to normal (50%-150%).

3. Administration of the Vectors Encoding Modified FIX Polypeptides

As described herein, the vectors generally are administered intravenously or by direct injection into the liver as detailed herein and known to those of skill in the art. The nucleic acids packaged in the capsid provided herein can be administered to a subject, including a subject having hemophilia, for therapy. An administered AAV vector packaged in the capsid provided herein can be any AAV described herein or any other AAV generated using the methods provided herein. In some examples, the AAV administered is an AAV containing a characteristic such as attenuated pathogenicity, low toxicity, preferential accumulation in the tissue or cells of interest (e.g., liver cells), low immunogenicity, replication competence and ability to express encoded proteins, and combinations thereof. The AAV viruses can be administered by direct injection into a compartmentalized liver (see, U.S. Pat. No. 9,821,114) in which the liver is clamped to isolate all or a portion from systemic circulation, the virus is injected into the parenchyma, compartmentalization is maintained for at least 25 or 30 minutes up to one hour to effect quantitative uptake of the injected virus.

### I. THERAPEUTIC USES

The vectors provided herein can be used for treatment of any condition for which unmodified FIX is employed. The modified polypeptides encoded in the vectors are designed to exhibit modified properties, such as improved pharmacokinetic and pharmacodynamic properties, increased resistance to inhibitors and/or improved catalytic activity. Such modified properties and activities, for example, improve the therapeutic effectiveness. This section provides exemplary uses of and administration methods. These described therapies are exemplary only and do not limit the applications of modified FIX polypeptides.

Among the uses for recombinant and modified coagulation factors are treatments of hemophilias. Hemophilia A is treated with FVIII, and Hemophilia B with FIX. Subjects with antibodies (inhibitors) against their replacement factor, generally against FVIII are treated with bypass agent: FVIIa or factor eight inhibitor bypass activity (FEIBA). The encoded modified FIX polypeptides described herein exhibit improved pharmacokinetic and pharmacodynamic properties, increased catalytic activity, increased resistance to inhibitors and/or increased coagulant activity compared to an unmodified FIX polypeptide. The encoded modified FIX polypeptides provided herein exhibit improved coagulant activity, as well as increased half-life and bioavailability, compared to unmodified FIX. Typically they are at least about 7-fold more potent than wild-type FIX products, such as BeneFIX® FIX, and as much or more than 20-fold more potent. This increased potency and the AAV vectors provided herein, such as the AAV with the capsid designated KP1, that have enhanced liver tropism, result in a therapeutic for gene therapy that can be effectively used for treating hemophilia to provide normal or near-normal coagulant activity or at least sufficient activity to result in mild hemophilia (greater than at least 10%, 20%, 30%, 40% up to 50% activity), or normal clotting (above about 40% or 50%).

Dosage levels and regimens can be determined based upon known dosages and regimens, and, if necessary can be extrapolated based upon the changes in properties of the modified polypeptides and/or can be determined empirically based on a variety of factors. Such factors include body weight of the individual, general health, age, the activity of the specific compound employed, sex, diet, time of administration, rate of excretion, drug combination, the severity and course of the disease, and the patient's disposition to the disease and the judgment of the treating physician.

The effect of the encoded FIX polypeptides on the clotting time of blood can be monitored using any of the clotting tests known in the art including, but not limited to, whole blood partial thromboplastin time (PTT), the activated partial thromboplastin time (aPTT), the activated clotting time (ACT), the recalcified activated clotting time, or the Lee-White Clotting time.

1. Hemophilia

Hemophilia is an ancient disease only brought under control in the last 50 years and is characterized by an inherited congenital tendency of males to bleed. Estimates, based on the World Federation of Hemophilia's (WFH) annual global surveys, indicate that the number of people with hemophilia in the world is approximately 190,000, 30,000 of which are affected by hemophilia B specifically. Hemophilia B, first described in 1952 (Biggs et al., (1952) British Medical Journal, 1378-1382) was named after Stephen Christmas, a five year old British boy and the first patient described with hemophilia B. Thus, hemophilia B is also referred to as “Christmas disease” to differentiate from the more prevalent hemophilia A, or “classic hemophilia”. Hemophilia B is a recessive X-linked blood coagulation disorder leading to a deficiency of functional factor IX, one of the serine proteases of the intrinsic pathway of the coagulation cascade of secondary hemostasis (see, FIG. 1) In hemophilia B, the deficiency of FIX results in the reduction of a functioning intrinsic tenase complex, leading to diminished thrombin generation and an inability to form and maintain a stable clot (Franchini et al. (2013) Biologics 7:33-38). Severe deficiency of FIX leads to recurrent hemarthroses and bleeding episodes in soft tissues and other organs (Escobar et al. (2013) J. Thromb. Haemost. 11:1449-1453).

Hemophilia is a bleeding disorder that is caused by a deficiency in one or more blood coagulation factors. It is characterized by a decreased ability to form blood clots at sites of tissue damage. Congenital X-linked hemophilias include hemophilia A and hemophilia B, or Christmas disease, which are caused by deficiencies in FVIII and FIX, respectively. Hemophilia A occurs at a rate of 1 out of 10,0000 males, while hemophilia B occurs in 1 out of 50,000 males.

Hemophilia B is the second most common form of hemophilia (approximately 20% of hemophilia cases); it is estimated to occur in one in 30,000 live male births across all ethnic groups. Because hemophilia is an X-linked, recessive condition, it occurs predominantly in males (Franchini et al. (2013) Biologics 7:33-38). Symptoms of hemophilia B include recurrent prolonged bleeding resulting from reduced levels or an absence of plasma FIX, whose function is to cleave and activate FX within the coagulation cascade (Goodeve (2015) J. Thromb. Haemost. 13:1184-1195). Existing treatment relies mainly on replacement therapy with clotting factors, either at the time of bleeding or as part of a prophylaxis schedule. The major complication of such therapy is the development of neutralizing antibodies, which is most frequently observed in patients affected with hemophilia A (Escobar et al. (2013) 1 Thromb. Haemost. 11:1449-1453).

Hemophilia B is a congenital bleeding disorder caused by a deficiency or structural abnormality of coagulation FIX. The FIX gene is located on the X chromosome and is therefore inherited as an X-linked recessive trait (Bowen (2002) J. Clin. Pathol. 55:127-144). Hemophilia B can also arise spontaneously without a positive family history which is the case in approximately 30% of affected individuals. Females that carry an X chromosome with a defective FIX gene are called carriers and they normally do not present with bleeding symptoms as their other X chromosome has a normal copy of the FIX gene, however random suppression of one of the X chromosomes during fetal development may result in symptomatic hemophilia B. Sons and daughters of a carrier female have a 50% chance of inheriting the disease-carrying X chromosome and thus to be affected by the disorder or to be a carrier female, respectively. All female offspring of an affected male will carry the defective FIX gene.

Patients with hemophilia suffer from recurring joint and muscle bleeds, which can be spontaneous or in response to trauma. The bleeding can cause severe acute pain, restrict movement, and lead to secondary complications including synovial hypertrophy. Furthermore, the recurring bleeding in the joints can cause chronic synovitis, which can cause joint damage, destroying synovium, cartilage, and bone.

A goal of gene therapy for treating hemophilia is for the treated subject to have normal clotting levels. In severe hemophilia, in which a subject has about 30 or more bleeds/year, the level of clotting activity is 1% or less; in moderate hemophilia the subject has about 15-20 bleeds/year, and clotting activity greater than about 1% and up to about 5%; normal in mild hemophilia a subject has clotting levels of greater than 5% up to about 40-50%. Normal clotting activity is above 40-50% clotting activity. A goal of gene therapy is to restore activity to normal or at least mild hemophilia, generally greater than 10%, 12%, 20%, 40% or higher activity.

2. Pathophysiology

Hemophilia B is a coagulation factor deficiency resulting from reduced levels or an absence of FIX. FIX is a vitamin K-dependent plasma protease that participates in the intrinsic blood coagulation pathway which occurs through a series of enzymatic reactions (see below and also FIG. 1 showing the intrinsic and extrinsic pathways of the coagulation cascade leading to Fibrin formation).

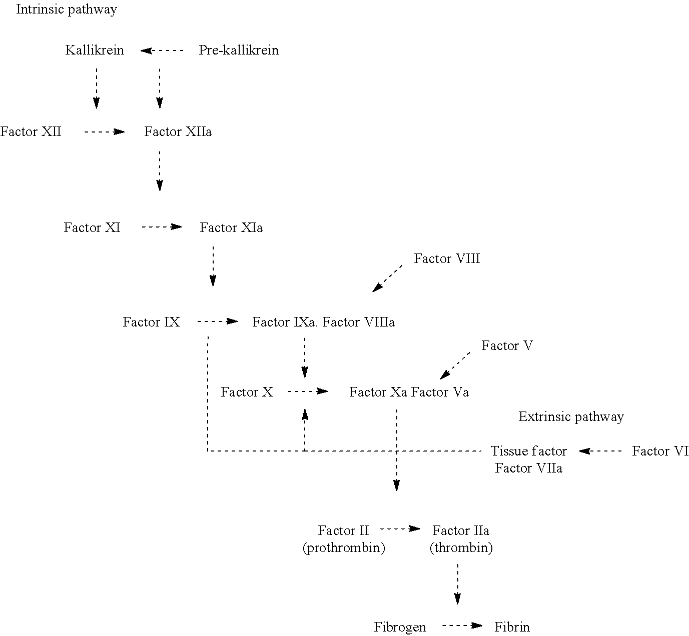

FVIII and FIX are synthesized in the liver and circulate as inactive precursors. They are activated, on demand, at the time of vascular injury, via the intrinsic or extrinsic pathways of the coagulation cascade. Factor VIII is a protein cofactor and factor IX is a serine protease which requires factor VIII as cofactor (Bowen (2002) J. Clin. Pathol. 55:127-144). Symptoms of recurrent prolonged bleeding result from reduced levels or an absence of plasma FIX, whose function is to cleave and activate FX within the coagulation cascade. FIX circulates as a zymogen, and is activated to activated FIX (FIXa) by sequential cleavage at p.Arg191-Ala192 and p.Arg226-Val227 by activated FXI or tissue factor-activated FVII. With activated FVIII as a cofactor providing correct orientation, FIXa cleaves FX resulting in its activation (Goodeve (2015) J. Thromb. Haemost. 13:1184-1195). In the common pathway, Factor Xa (generated through the intrinsic or extrinsic pathways) forms a prothrombinase complex with phospholipids, calcium ions, and thrombin-activated Factor Va. The complex cleaves prothrombin into thrombin and prothrombin fragments 1 and 2. Thrombin converts fibrinogen into fibrin, the structural polymer of the blood

In patients with hemophilia B, the activation of factor X is compromised due to the insufficient activity of the tenase complex brought about by deficiency of FIX enzyme activity. This significantly impairs clot formation and, as a consequence, results in spontaneous hemorrhage and/or prolonged bleeding episodes in response to injury or trauma (Bowen (2002) J. Clin. Pathol. 55:127-144). Apart from the functional differences of FVIII and FIX, there are other differences in the pathophysiology of hemophilia A compared with hemophilia B. One major difference is the half-life (T½) of the impacted protein. FVIII has a half-life (t½) 8 to 14 hours, while that of Factor IX is 18 to 24 hours.

3. Clinical Characteristics

Hemophilia B is characterized by a deficiency in FIX clotting activity that results in delayed or recurrent bleeding prior to complete wound healing after injuries, tooth extractions or surgery. Muscle hematomas or intracranial bleeding can occur immediately or up to four to five days after the original injury. Intermittent oozing may last for days or weeks after tooth extraction. Prolonged or delayed bleeding or wound hematoma formation after surgery is common. After circumcision, males with hemophilia B of any severity may have prolonged oozing, or they may heal normally. In severe hemophilia B, spontaneous joint bleeding is the most frequent symptom. The severity of bleeding manifestations in hemophilia is generally correlated with the clotting factor level as shown in Table 14, below. In patients with severe hemophilia, when untreated, bleeding in the joints may occur as frequently as 30-50 times a year.

TABLE 14
Correlation of Clotting Factor Levels with Severity of Hemophilia

| | Clotting | |
|---|---|---|
| | factor level | |
| | % activity | |
| Severity | (IU/ml) | Bleeding episodes |
| Severe | <1 IU/dl | Spontaneous bleeding, predominantly in joints and muscles and |
| | (<0.01 | often in the absence of identifiable hemostatic challenge. Usually |
| | IU/ml) | diagnosed during the first two years of life; without prophylactic |
| | or <1% | treatment, they may average up to two to five spontaneous |
| | of normal | bleeding episodes each month. |
| Moderate | 1-5 IU/dl | Occasional spontaneous bleeding. Prolonged bleeding with |
| | (0.01- | trauma and surgery. Usually diagnosed before age five to six |
| | 0.05 IU/ml) | years; the frequency of bleeding episodes varies from once a |
| | or 1-5% | month to once a year. |
| | of normal | |
| Mild | 5-40 IU/dl | Severe bleeding with major trauma or surgery without pre- and |
| | (0.05- | post-operative treatment. Spontaneous bleeding is rare; the |
| | 0.40 IU/ml) | frequency of bleeding may vary from once a year to once every ten |
| | or 5-<40% | years. Individuals with mild hemophilia are often not diagnosed |
| | of normal | until later in life. |

Source: Adapted from (Konkle et al., 2000; World Federation of Hemophilia - Guidelines for the management of hemophilia 2nd edition, 2013)

4. Hemophilia B

Hemophilia B can be effectively managed with administration of FIX therapeutics. Patients with severe Hemophilia B have an FIX activity of <1 IU/dL (1% of normal activity), patients with moderate Hemophilia B have a FIX activity of 1-5 IU/dL (1-5% of normal activity) and patients with mild hemophilia B have a FIX activity of >5-<40 IU/mL (>5-<40% of normal activity). With proper prophylactic replacement therapy and/or treatment of particular bleeding episodes with an appropriate amount of FIX, patients often can achieve normal life span. Administration of FIX can aid in controlling bleeding during surgery, trauma, during dental extraction, or to alleviate bleeding associated with hemarthrosis, hematuria, mucocutaneous bleeding, such as epistaxis or gastrointestinal tract bleeding, cystic lesions in subperiosteal bone or soft tissue, or hematomas, which cause neurological complications such as intracranial bleeding, and spinal canal bleeding. Death in patients with hemophilia is often the result of bleeding in the central nervous system. Other serious complications in hemophilic patients include development of inhibitors to coagulation factor therapeutics and disease.

The most frequent alterations in the FIX gene in hemophilia B patients are point mutations, in particular missense mutations. Most of the identified FIX mutations occur in amino acid residues in the coding region of the FIX gene, often affecting evolutionarily conserved amino acids. The severity of the hemophilia depends upon the nature of the mutation. Mutations in the coding region can affect a number of different properties or activities of the FIX polypeptide including alteration of protease activity, cofactor binding, signal peptide or propeptide cleavage, post-translational modifications, and inhibition of cleavage of FIX into its activated form. Other types of point mutations include nonsense mutations that produce an unstable truncated FIX polypeptide, and frameshift mutations (small deletions and insertions) that result in a terminally aberrant FIX molecule. In addition, FIX point mutations can be found in the promoter region, which can disrupt the recognition sequences for several specific gene regulatory proteins, resulting in reduced transcription of coagulation factor IX. Decreased FIX as a result of transcriptional abnormalities is called Hemophilia B Leyden. An exemplary mutation in the promoter region includes disruption of the HNF-4 binding site, which affect regulation of FIX transcription by the androgen receptor. The severity of this type of hemophilia is governed by the levels of androgen in the blood, which increase during puberty and partially alleviate the FIX transcriptional deficiency (Kurachi and Kurachi (1995) Thrombosis and Haemostasis 73(3):333-339). Other missense nucleotide changes affect the processing of factor IX primary RNA transcript. For example, some mutations occur at evolutionarily conserved donor-splice (GT), and acceptor-splice (AG) consensus sequences, which can create cryptic splice junctions and disrupt assembly of spliceosomes. Some severe cases of hemophilia (approximately 10%) present with large deletions in the FIX gene.

Treatment of FIX deficiency, and thus hemophilia B, most often involves administration of FIX, including recombinant forms of FIX, purified plasma FIX preparations or purified plasma concentrates. Thus, similarly, the modified FIX polypeptides herein, and nucleic acids encoding modified FIX polypeptides, can be used for treatment of hemophilia B. The modified FIX polypeptides herein can exhibit improved pharmacokinetic and pharmacodynamic properties, such as improved serum half-life, increased resistance to inhibitors, increased catalytic activity, and/or increased coagulant activity. Thus, modified FIX polypeptides can be used to deliver improved therapies for hemophilia. Examples of therapeutic improvements using modified FIX polypeptides include for example, but are not limited to, lower dosages, fewer and/or less frequent administrations, decreased side effects, and increased therapeutic effects.

Hemophilia B is an X-linked genetic disease caused by a mutation in the gene of coagulation factor IX (FIX). Hemophilia B patients have spontaneous internal bleeding occurring mainly in muscles and joints, resulting in chronic joint injury, and have poor hemostasis. Hemophilia B severity is categorized as mild (5%-40% of normal blood FIX activity; 5-40 IU/dL), moderate (1%-5%; 1-5 IU/dL), and severe (<1%; <1 IU/dL). Treatment of bleeding episodes in type B hemophilia has accomplished by supplementing FIX by intravenous (IV) injection. FIX products used for treatment of bleeding episodes include plasma-derived FIX (isolated and concentrated from human blood) and recombinant wild-type FIX (rwt-FIX), which is more desirable due to concerns of infection by human viruses with plasma-derived FIX. During hemostasis, the dose of FIX administered varies according to the severity of the bleeding and the weight of the patient.

### J. COMBINATION THERAPIES

The gene therapy provided herein can be supplemented with other treatments, including FIX polypeptide treatment, and other therapeutic agents or procedures including, but not limited to, other biologics, small molecule compounds and surgery. For any disease or condition, including all those exemplified above, for FIX is indicated or has been used and for which other agents and treatments are available, FIX can be used in combination therewith. Hence, the modified FIX polypeptides provided herein similarly can be used. Depending on the disease or condition to be treated, exemplary combinations include, but are not limited to combination with other plasma purified or recombinant coagulation factors, procoagulants, anticoagulants, anti-coagulation antibodies, glycosaminoglycans, heparins, heparinoids, heparin derivatives, heparin-like drugs, coumarins, such as warfarin, and coumarin derivatives. Additional procoagulants that hat have procoagulant properties can be administered. These include, but are not limited to, vitamin K, vitamin K derivatives, other coagulation factors, and protein C inhibitors. Additional anticoagulants that can be used in combination therapies with modified FIX polypeptides provided herein that have anticoagulant properties include, but are not limited to, β2 adrenoreceptor antagonists, neuropeptide V2 antagonists, prostacyclin analogs, thromboxane synthase inhibitors, calcium agonists, elastase inhibitors, non-steroidal anti-inflammatory molecules, thrombin inhibitors, lipoxygenase inhibitors, FVIIa inhibitors, FXa inhibitors, phosphodiesterase III inhibitors, fibrinogen, vitamin K antagonists, and glucoprotein IIb/IIIa antagonists.

### K. EXAMPLES

Examples 1-9 are reproduced from U.S. Pat. Nos. 9,328,339 and 8,778,870, which describe variants of FIX (modified FIX) polypeptides that have increased coagulation activity. Among these also are modified FIX polypeptides that have increased resistance to an endogenous protease or proteases and/or increased coagulation activity. Provided herein are constructs that contain nucleic acid encoding any these modified FIX that have increased coagulation activity and/or increased resistance to an endogenous protease. The constructs include ITRs from AAV and regulatory sequences for gene therapy for expression in an animal, such as a human. The FIX encoding nucleic acid includes an intron, which increases expression in an animal. AAV capsids containing the constructs also are provided. The capsids have tropism for transducing hepatocytes so that the encoded FIX, when administered transduce the liver. The constructs include liver-specific promoters, so that the encoded FIX is expressed in liver. The capsids are recombinant AAV capsids that are generated and selected to have increased tropism for liver compared to any AAV serotype, include AAV8, and also compared to the recombinant AAV, designated DJ/8 (or DJ8). Among these are capsids that have tropism for liver, and also have tropism for islet cells. Examples 10-16 describe the generation of the capsids, and their use to encode and express modified FIX for gene therapy.

### Example 1

### Cloning and Expression of Factor IX Polypeptides

### A. Cloning of FIX Gene

The nucleic acid encoding the 461 amino acid human FIX precursor polypeptide (P00740; set forth in SEQ ID NO:1) was cloned into the mammalian expression vector, pFUSE-hIgG1-Fc2 (abbreviated here as pFUSE) (InvivoGen; SEQ ID NO:23), which contains a composite promoter, hEF1-HTLV, comprising the Elongation Factor-1α (EF-1α) core promoter and the R segment and part of the U5 sequence (R-U5′) of the human T-Cell Leukemia Virus (HTLV) Type 1 Long Terminal Repeat. The In-Fusion CF Dry-Down PCR Cloning Kit (Clontech) was used according to the conditions specified by the supplier.

For the In-Fusion process, plasmid pFUSE without the human immunoglobulin 1 (hIgG1) Fc portion was linearized using polymerase chain reaction (PCR) with the pFUSE-Acc-F1 forward primer: GTGCTAGCTGGCCAGACATGATAAG (SEQ ID NO:24) and the pFUSE-Acc-R3 reverse primer: CATGGTGGCCCTCCTTCGCCGGTGATC (SEQ ID NO:25), and was used as Acceptor DNA. The full-length coding sequence of FIX was amplified by PCR using human FIX cDNA (Origene) as template with the FIX-wtsp-Invivo-F1 forward primer: CGAAGGAGGGCCACCATGCAGCGCGTGAACATGATC (SEQ ID NO:26) and FIX-Invivo-R1 reverse primer: TGTCTGGCCAGCTAGCACTTAAGTGAGCTTTGTTTTTTCC (SEQ ID NO:27). For two FIX Donor amplification primer sequences set forth above, both FIX ‘ATG’ start and complementary sequence of ‘TAA’ stop codons are underlined in the forward and reverse primer sequences, respectively. The 18-nt long homology regions, a non-annealing 5′ primer tail for In-Fusion, are shown in bold. Standard PCR reaction and thermocycling conditions were used in conjunction with the Phusion High-Fidelity Master Mix Kit (New England Biolabs), as recommended by the manufacturer. Both Acceptor and Donor PCR products were then digested with DpnI restriction enzyme to remove E. coli-derived dam methylated PCR template backgrounds. They were then mixed together, and the In-Fusion reaction was run using conditions specified by the supplier. The reaction mix was transformed into E. coli XL1Blue supercompetent cells (Stratagene). Colonies were selected on 2xYT agar plates supplemented with 25 ppm Zeocin (InvivoGen). Plasmid DNA was isolated from selected clones, and sequenced to verify correct cloning.

### B. Generation of FIX Variants

FIX variants were generated using the QuikChange Lightning Site-Directed Mutagenesis Kit (Stratagene) according to manufacturer's instructions with specifically designed oligonucleotides that served as primers to incorporate designed mutations into the newly synthesized DNA. Complementary primers that include the desired mutations were extended during cycling using purified, double-stranded super-coiled pFUSE plasmid DNA that contained the cloned FIX cDNA sequence as a template. Extension of the primers resulted in incorporation of the mutations of interest into the newly synthesized strands, and resulted in a mutated plasmid with staggered nicks. Following amplification, the mutagenesis product was digested with DpnI restriction enzyme to remove dam methylated parental strands of the E. coli-derived pFUSE DNA. The DNA was then transformed into E. coli XL1Blue supercompetent cells (Stratagene) followed by selection on 2xYT agar plates supplemented with 25 ppm Zeocin (InvivoGen). Plasmid DNA was isolated from selected clones, and sequenced to verify for incorporation of mutation(s) at the desired location(s) on the FIX gene.

The nucleotide sequence of one of the oligonucleotides from each complementary primer pair used to generate the FIX variants is provided in Table 15. The nucleotide triplet sequences that encode a substituted amino acid are shown in uppercase. For example, to generate a FIX variant containing the substitutions A103N/N105S (A[103]N/N[105]S by chymotrypsin numbering; SEQ ID NO:77), the A103N/N105S-Forward primer, and a primer that is complementary to A103N/N105S-Forward, were used to replace a 9-bp ‘GCTgatAAC’ wild-type sequence with a 9-bp ‘AATgatAGC’ mutant sequence (changed nucleotide triplets are denoted by upper case).

Table 15, below, sets forth the oligonucleotide primers used for FIX mutagenesis. The mutant triplets are shown in upper case, and primer names correspond to the mutation, by chymotrypsin numbering, produced as a result of the mutagenesis using the primer.

TABLE 15
Oligonucleotide Primers for FIX Mutagenesis

| | | SEQ |
|---|---|---|
| Primer Name | Primer Sequence (5′ to 3′) | ID NO. |
| F9-A[103]N/ | gtaaaaatagtAATgatAGCaaggtggtttg | 28 |
| N[105]S-For | | |
| F9-D[104]N/ | gtaaaaatagtgctAATaacAGTgtggtttgctoctgtactg | 29 |
| K[106]S-For | | |
| F9-K[106]N/ | gtgctgataacAATgtgAGTtgctoctgtactg | 30 |
| V[108]S-For | | |
| F9-D[85]N-For | gaactgtgaattaAATgtaacatgtaac | 31 |
| F9-T[148]A-For | ctcacccgtgctgagGCTgtttttcctgatgtg | 32 |
| F9-D39N/F41T-For | gaatggtaaagttAATgcaACCtgtggaggctctatc | 33 |
| F9-K63N-For | gaaactggtgttAACattacagttgtcgc | 34 |
| F9-I86S-For | gcgaaatgtgAGTcgaattattcctc | 35 |
| F9-A95bS-For | caactacaatgcaAGTattaataagtacaac | 36 |
| F9-K243N-For | aaggaaaaaacaAATctcacttaagtgctagctg | 37 |
| F9-E240N-For | ctggattaagAATaaaacaaagctc | 38 |
| F9-E74N-For | caggtgaacataatattAACgagacagaacatacag | 39 |
| F9-T76N/H78S-For | gaacataatattgaggagAACgaaAGTacagagcaaaag | 40 |
| F9-K82N/N845-For | cagaacatacagagcaaAATcgaTCTgtgattcgaattattc | 41 |
| F9-L153N-For | gggagatcagctAATgttcttcagtac | 42 |
| F9-F145N/H147S- | ctggggaagagtcAACTCCaaagggagatcag | 43 |
| For | | |
| F9-K222N/K224S- | gagtgtgcaatgAACggcTCAtatggaatatatac | 44 |
| For | | |
| F9-S151N/L153S- | cttccacaaagggagaAATgctTCAgttcttca | 45 |
| For | | |
| F9-N95S-For | cctcaccacaactacAGTgcagctattaataagtacaacc | 46 |
| F9-Y117N-For | cttagtgctaaacagcAACgttacacctatttgc | 47 |
| F9-G149N-For | ggaagagtcttccacaaaAACagatcagctttagttc | 48 |
| F9-R150N/A152S- | gtcttccacaaagggAACtcaTCTttagttcttcagtac | 49 |
| For | | |
| F9-R150A-For | gtcttccacaaagggGCAtcagctttagttcttcag | 50 |
| F9-R150E-For | gtcttccacaaagggGAAtcagctttagttcttcag | 51 |
| F9-R150Y-For | gtcttccacaaagggTACtcagctttagttcttcag | 52 |
| F9-R143Q-For | gtaagtggctggggaCAAgtcttccacaaaggg | 53 |
| F9-R143A-For | gtaagtggctggggaGCAgtcttccacaaaggg | 54 |
| F9-R143Y-For | gtaagtggctggggaTACgtcttccacaaaggg | 55 |
| F9-R143L-For | gtaagtggctggggaCTGgtcttccacaaaggg | 56 |
| F9-V38M-For | gttttgaatggtaaaATGgatgcattctgtggaggc | 57 |
| F9-V38Y-For | gttttgaatggtaaaTACgatgcattctgtggaggc | 58 |
| F9-D39M-For | gttttgaatggtaaagttATGgcattctgtggaggc | 59 |
| F9-D39Y-For | gttttgaatggtaaagttTACgcattctgtggaggc | 60 |
| F9-A40M-For | gttttgaatggtaaagttgatATGttctgtggaggctctatc | 61 |
| F9-A40Y-For | gttttgaatggtaaagttgatTACttctgtggaggctctatc | 62 |
| F9-R233A/K230A- | caaatatggaatatataccGCAgtatccGCAtatgtcaactgg | 63 |
| For | attaag | |
| F9-R233E/K230E- | caaatatggaatatataccGAAgtatccGAAtatgtcaactgg | 64 |
| For | attaag | |
| F9-R233A-For | gaatatataccaaggtatccGCAtatgtcaactggattaag | 65 |
| F9-R233E-For | gaatatataccaaggtatccGAAtatgtcaactggattaag | 66 |
| F9-K230A-For | caaatatggaatatataccGCAgtatcccggtatgtc | 67 |
| F9-K230E-For | caaatatggaatatataccGAAgtatcccggtatgtc | 68 |
| F9-K126E-For | cctatttgcattgctgacGAAgaatacacgaacatc | 69 |
| F9-K126A-For | cctatttgcattgctgacGCAgaatacacgaacatc | 70 |
| F9-R165A-For | gttccacttgttgacGCAgccacatgtcttcgatct | 71 |
| F9-R165E-For | gttccacttgttgacGAAgccacatgtcttcgatct | 72 |
| F9-R170A-For | cgagccacatgtcttGCAtctacaaagttcacc | 73 |
| F9-R170E-For | cgagccacatgtcttGAAtctacaaagttcacc | 74 |
| F9-D[64]N-For | ggcggcagttgcaagAACgacattaattcctatG | 273 |
| F9-D[64]A-For | ggcggcagttgcaagGCTgacattaattcctatG | 274 |
| F9-N[157]Q-For | cctgatgtggactatgtaCAGtctactgaagctgaaacc | 275 |
| F9-N[157]D-For | cctgatgtggactatgtaGACtctactgaagctgaaacc | 276 |
| F9-N[167]Q-For | gaaaccattttggatCAGatcactcaaagcacc | 277 |
| F9-N[167]D-For | gaaaccattttggatGACatcactcaaagcacc | 278 |
| F9-S[61]A-For | ccatgtttaaatggcggcGCTtgcaaggatgacattaattcc | 279 |
| F9-S[53]A-For | gatggagatcagtgtgagGCTaatccatgtttaaatggc | 280 |
| F9-T[159]A-For | gtggactatgtaaattctGCTgaagctgaaaccattttg | 281 |
| F9-T[169]A-For | CattttggataacatcGCTcaaagcacccaatcatttaatgac | 282 |
| F9-T[172]A-For | gataacatcactcaaagcGCTcaatcatttaatgac | 283 |
| F9-T[179]A-For | caatcatttaatgacttcGCTcgggttgttggtggagaaG | 284 |
| F9-Y[155]F-For | gtttttcctgatgtggacTTCgtaaattctactgaagctG | 285 |
| F9-Y[155]H-For | gtttttcctgatgtggacCACgtaaattctactgaagctG | 286 |
| F9-Y[155]Q-For | gtttttcctgatgtggacCAGgtaaattctactgaagctG | 287 |
| F9-S[158]A-For | gtggactatgtaaatGCTactgaagctgaaacc | 288 |
| F9-S[158]D-For | gtggactatgtaaatGACactgaagctgaaacc | 289 |
| F9-S[158]E-For | gtggactatgtaaatGAGactgaagctgaaacc | 290 |
| F9-R165S-For | gttccacttgttgacAGCgccacatgtottcgatct | 291 |
| F9-R170L-For | cgagccacatgtcttCTGtctacaaagttcacc | 292 |
| F9-K148N-For | ggaagagtottccacAACgggagatcagotttaG | 293 |
| F9-K148A-For | ggaagagtcttccacGCTgggagatcagctttaG | 294 |
| F9-K148E-For | ggaagagtcttccacGAGgggagatcagctttaG | 295 |
| F9-K148S-For | ggaagagtottccacAGCgggagatcagotttaG | 296 |
| F9-K148M-For | ggaagagtottccacATGgggagatcagotttaG | 297 |
| F9-E74S-For | ggtgaacataatattAGCgagacagaacatacaG | 298 |
| F9-E74A-For | ggtgaacataatattGCTgagacagaacatacaG | 299 |
| F9-E74R-For | ggtgaacataatattAGGgagacagaacatacaG | 300 |
| F9-E74K-For | ggtgaacataatattAAGgagacagaacatacaG | 301 |
| F9-H92F-For-Corr | cgaattattcctcacTTCaactacaatgcaGC | 302 |
| F9-H92Y-For-Corr | cgaattattcctcacTACaactacaatgcaGC | 303 |
| F9-H92E-For-Corr | cgaattattcctcacGAAaactacaatgcaGC | 304 |
| F9-H92S-For-Corr | cgaattattcctcacAGCaactacaatgcaGC | 305 |
| F9-T242A-For | CtggattaaggaaaaaGCTaagctcacttaagtg | 306 |
| F9-T242V-For | CtggattaaggaaaaaGTGaagctcacttaagtg | 307 |
| F9-E240N/T242A- | gtcaactggattaagAACaaaGCTaagctcacttaagtg | 308 |
| For | | |
| F9-E240N/T242V- | gtcaactggattaagAACaaaGTGaagctcacttaagtg | 309 |
| For | | |
| F9-E240Q-For | gtcaactggattaagCAGaaaacaaagctcacttaaG | 310 |
| F9-E240S-For | gtcaactggattaagAGCaaaacaaagctcacttaaG | 311 |
| F9-E240A-For | gtcaactggattaagGCTaaaacaaagctcacttaaG | 312 |
| F9-E240D-For | gtcaactggattaagGACaaaacaaagctcacttaaG | 313 |
| F9-N178D-For | CAaagttcaccatctatGACaacatgttctgtgctggc | 314 |
| F9-N178Y-For | CAaagttcaccatctatTACaacatgttctgtgctggc | 315 |
| F9-Y177A-For | CTacaaagttcaccatcGCTaacaacatgttctgtGC | 316 |
| F9-Y177T-For | CTacaaagttcaccatcACCaacaacatgttctgtGC | 317 |
| F9-T175R-For | cttcgatctacaaagttcAGGatctataacaacatgttc | 318 |
| F9-T175E-For | cttcgatctacaaagttcGAAatctataacaacatgttc | 319 |
| F9-T175Q-For | cttcgatctacaaagttcCAGatctataacaacatgttc | 320 |
| F9-F174I-For | GTcttcgatctacaaagATCaccatctataacaacatg | 321 |
| F9-T175R/Y177T- | cgatctacaaagttcAGGatcACCaacaacatgttctgtG | 322 |
| For | | |
| F9-Y94F/K98T-For | GAattattcctcaccacaacTTCaatgcagctattaatACCta | 323 |
| | caaccatgacattG | |
| F9-F145N/K1485- | ggctggggaagagtcAACcacAGCgggagatcagotttaG | 324 |
| For | | |

Table 16, below, sets forth the FIX variants that were generated, with the mutations indicated using numbering relative to the mature FIX polypeptide set forth in SEQ ID NO:3, and also chymotrypsin numbering.

TABLE 16
FIX variants

| Mutation | Mutation | SEQ |
|---|---|---|
| (Mature FIX | (Chymotrypsin | ID |
| Numbering) | Numbering) | NO. |
| Catalyst Biosciences WT | Catalyst Biosciences WT | 3 |
| N157D | N[157]D | 75 |
| Y155F | Y[155]F | 76 |
| A103N/N105S | A[103]N/N[105]S | 77 |
| D104N/K106S | D[104]N/K[106]S | 78 |
| K106N/V108S | K[106]N/V[108]S | 79 |
| D85N | D[85]N | 80 |
| T148A | T[148]A | 81 |
| K5A | K[5]A | 82 |
| D64N | D[64]N | 83 |
| D64A | D[64]A | 84 |
| N167D | N[167]D | 85 |
| N167Q | N[167]Q | 86 |
| S61A | S[61]A | 87 |
| S53A | S[53]A | 88 |
| T159A | T[159]A | 89 |
| T169A | T[169]A | 90 |
| T172A | T[172]A | 91 |
| T179A | T[179]A | 92 |
| Y155H | Y[155]H | 93 |
| Y155Q | Y[155]Q | 94 |
| S158A | S[158]A | 95 |
| S158D | S[158]D | 96 |
| S158E | S[158]E | 97 |
| N157Q | N[157]Q | 98 |
| D203N/F205T | D39N/F41T | 99 |
| D85N/D203N/F205T | D[85]N/D39N/F41T | 100 |
| K228N | K63N | 101 |
| D85N/K228N | D[85]N/K63N | 102 |
| I251S | I86S | 103 |
| D85N/I251S | D[85]N/I86S | 104 |
| D85N/D104N/K106S/I251S | D[85]N/D[104]N/K[106]S/I86S | 105 |
| A262S | A95bS | 106 |
| K413N | K243N | 107 |
| E410N | E240N | 108 |
| E239N | E74N | 109 |
| T241N/H243S | T76N/H78S | 110 |
| K247N/N249S | K82N/N84S | 111 |
| L321N | L153N | 112 |
| F314N/H315S | F145N/H147S | 113 |
| K392N/K394S | K222N/K224S | 114 |
| S319N/L321S | S151N/L153S | 115 |
| N260S | N95S | 116 |
| Y284N | Y117N | 117 |
| G317N | G149N | 118 |
| R318N/A320S | R150N/A152S | 119 |
| R318A | R150A | 120 |
| R318E | R150E | 121 |
| R318Y | R150Y | 122 |
| R312Q | R143Q | 123 |
| R312A | R143A | 124 |
| R312Y | R143Y | 125 |
| R312L | R143L | 126 |
| V202M | V38M | 127 |
| V202Y | V38Y | 128 |
| D203M | D39M | 129 |
| D203Y | D39Y | 130 |
| A204M | A40M | 131 |
| A204Y | A40Y | 132 |
| K400A/R403A | K230A/R233A | 133 |
| K400E/R403E | K230E/R233E | 134 |
| R403A | R233A | 135 |
| R403E | R233E | 136 |
| K400A | K230A | 137 |
| K400E | K230E | 138 |
| K293E | K126E | 139 |
| K293A | K126A | 140 |
| R333A | R165A | 141 |
| R333E | R165E | 142 |
| R338A | R170A | 143 |
| R338E | R170E | 144 |
| R338A/R403A | R170A/R233A | 145 |
| R338E/R403E | R170E/R233E | 146 |
| K293A/R403A | K126A/R233A | 147 |
| K293E/R403E | K126E/R233E | 148 |
| K293A/R338A/R403A | K126A/R170A/R233A | 149 |
| K293E/R338E/R403E | K126E/R170E/R233E | 150 |
| R318A/R403A | R150A/R233A | 151 |
| R318E/R403E | R150E/R233E | 152 |
| R318Y/E410N | R150Y/E240N | 153 |
| R338E/E410N | R170E/E240N | 154 |
| R338E/R403E/E410N | R170E/R233E/E240N | 155 |
| R318Y/R338E/R403E | R150Y/R170E/R233E | 156 |
| D203N/F205T/K228N | D39N/F41T/K63N | 157 |
| D203N/F205T/E410N | D39N/F41T/E240N | 158 |
| D203N/F205T/R338E | D39N/F41T/R170E | 159 |
| D203N/F205T/R338A | D39N/F41T/R170A | 160 |
| D203N/F205T/R318Y | D39N/F41T/R150Y | 161 |
| D203N/F205T/R338E/R403E | D39N/F41T/R170E/R233E | 162 |
| K228N/E410N | K63N/E240N | 163 |
| K228N/R338E | K63N/R170E | 164 |
| K228N/R338A | K63N/R170A | 165 |
| K228N/R318Y | K63N/R150Y | 166 |
| K228N/R338E/R403E | K63N/R170E/R233E | 167 |
| R403E/E410N | R233E/E240N | 168 |
| R318Y/R338E/E410N | R150Y/R170E/E240N | 169 |
| K228N/R318Y/E410N | K63N/R150Y/E240N | 170 |
| R318Y/R403E/E410N | R150Y/R233E/E240N | 171 |
| R318Y/R338E/R403E/E410N | R150Y/R170E/R233E/E240N | 172 |
| D203N/F205T/R318Y/E410N | D39N/F41T/R150Y/E240N | 173 |
| R333S | R165S | 186 |
| R338L | R170L | 187 |
| K316N | K148N | 189 |
| K316A | K148A | 190 |
| K316E | K148E | 191 |
| K316S | K148S | 192 |
| K316M | K148M | 193 |
| E239S | E74S | 194 |
| E239A | E74A | 195 |
| E239R | E74R | 196 |
| E239K | E74K | 197 |
| H257F | H92F | 198 |
| H257Y | H92Y | 199 |
| H257E | H92E | 200 |
| H257S | H92S | 201 |
| T412A | T242A | 202 |
| T412V | T242V | 203 |
| E410N/T412A | E240N/T242A | 204 |
| E410N/T412V | E240N/T242V | 205 |
| E410Q | E240Q | 174 |
| E410S | E240S | 175 |
| E410A | E240A | 176 |
| E410D | E240D | 206 |
| N346D | N178D | 207 |
| N346Y | N178Y | 208 |
| F314N/K316S | F145N/K148S | 177 |
| A103N/N105S/K228N | A[103]N/N[105]S/K63N | 217 |
| D104N/K106S/K228N | D[104]N/K[106]S/K63N | 218 |
| K228N/I251S | K63N/I86S | 180 |
| A103N/N105S/I251S | A[103]N/N[105]S/I86S | 181 |
| D104N/K106S/I251S | D[104]N/K[106]S/I86S | 182 |
| A103N/N105S/R318Y/R338E/R403E/ | A[103]N/N[105]S/R150Y/R170E/ | 219 |
| E410N | R233E/E240N | |
| D104N/K106S/R318Y/R338E/R403E/ | D[104]N/K[106]S/R150Y/R170E/ | 220 |
| E410N | R233E/E240N | |
| K228N/R318Y/R338E/R403E/E410N | K63N/R150Y/R170E/R233E/E240N | 221 |
| I251S/R318Y/R338E/R403E/E410N | I86S/R150Y/R170E/R233E/E240N | 222 |
| D104N/K106S/I251S/R318Y/R338E/ | D[104]N/K[106]S/I86S/R150Y/ | 223 |
| R403E/E410N | R170E/R233E/E240N | |
| D104N/K106S/R318Y/E410N/R338E | D[104]N/K[106]S/R150Y/E240N/ | 224 |
| | R170E | |
| I251S/R318Y/E410N/R338E | I86S/R150Y/E240N/R170E | 225 |
| D104N/K106S/I251S/R318Y/R338E/ | D[104]N/K[106]S/I86S/R150Y/ | 226 |
| E410N/ | R170E/E240N | |
| A103N/N105S/K247N/N249S | A[103]N/N[105]S/K82N/N84S | 178 |
| D104N/K106S/K247N/N249S | D[104]N/K[106]S/K82N/N84S | 179 |
| K228N/K247N/N249S | K63N/K82N/N84S | 183 |
| A103N/N105S/Y155F | A[103]N/N[105]S/Y[155]F | 227 |
| D104N/K106S/Y155F | D[104]N/K[106]S/Y[155]F | 228 |
| Y155F/K228N | Y[155]F/K63N | 229 |
| Y155F/I251S | Y[155]F/I86S | 230 |
| Y155F/K247N/N249S | Y[155]F/K82N/N84S | 231 |
| A103N/N105S/K247N/N249S/R318Y/ | A[103]N/N[105]S/K82N/N84S/ | 232 |
| R338E/R403E/E410N | R150Y/R170E/R233E/E240N | |
| D104N/K106S/K247N/N249S/R318Y/ | D[104]N/K[106]S/K82N/N84S/ | 233 |
| R338E/R403E/E410N | R150Y/R170E/R233E/E240N | |
| K228N/K247N/N249S/R318Y/R338E/ | K63N/K82N/N84S/R150Y/R170E/ | 234 |
| R403E/E410N | R233E/E240N | |
| A103N/N105S/Y155F/R318Y/R338E/ | A[103]N/N[105]S/Y[155]F/R150Y/ | 235 |
| R403E/E410N | R170E/R233E/E240N | |
| D104N/K106S/Y155F/R318Y/R338E/ | D[104]N/K[106]S/Y[155]F/ | 236 |
| R403E/E410N | R150Y/R170E/R233E/E240N | |
| Y155F/K228N/R318Y/R338E/R403E/ | Y[155]F/K63N/R150Y/R170E/ | 237 |
| E410N | R233E/E240N | |
| Y155F/I251S/R318Y/R338E/R403E/ | Y[155]F/I86S//R150Y/R170E/ | 238 |
| E410N | R233E/E240N | |
| Y155F/K247N/N249S/R318Y/R338E/ | Y[155]F/K82N/N84S/R150Y/R170E/ | 239 |
| R403E/E410N | R233E/E240N | |
| K247N/N249S/R318Y/R338E/R403E/ | K82N/N84S/R150Y/R170E/R233E/ | 240 |
| E410N | E240N | |
| Y155F/R318Y/R338E/R403E/E410N | Y[155]F/R150Y/R170E/R233E/E240N | 241 |
| K247N/N249S/R318Y/R338E/E410N | K82N/N84S/R150Y/R170E/E240N | 242 |
| Y155F/R318Y/R338E/E410N | Y[155]F/R150Y/R170E/E240N | 243 |
| Y155F/K247N/N249S/R318Y/R338E/ | Y[155]F/K82N/N84S/R150Y/R170E/ | 244 |
| E410N | E240N | |
| D104N/K106S/YE55F/K228N/K247N/ | D[104]N/K[106]S/Y[155]F/ | 245 |
| N249S | K63N/K82N/N84S | |
| D104N/K106S/Y155F/K247N/N249S | D[104]N/K[106]S/Y[155]F/K82N/N84S | 246 |
| D104N/K106S/Y155F/K228N/ | D[104]N/K[106]S/Y[155]F/K63N | 247 |
| Y155F/K228N/K247N/N249S | Y[155]F/K63N/K82N/N84S | 248 |
| D104N/K106S/K228N/K247N/N249S | D[104]N/K[106]S/K63N/K82N/N84S | 184 |
| R318Y/R338E/R403E/E410S | R150Y/R170E/R233E/E240S | 249 |
| R318Y/R338E/R403E/E410N/T412V | R150Y/R170E/R233E/E240N/T242V | 250 |
| R318Y/R338E/R403E/E410N/T412A | R150Y/R170E/R233E/E240N/T242A | 251 |
| R318Y/R338E/R403E/T412A | R150Y/R170E/R233E/T242A | 252 |
| R318Y/R338E/E410S | R150Y/R170E/E240S | 253 |
| R318Y/R338E/T412A | R150Y/R170E/T242A | 254 |
| R318Y/R338E/E410N/T412V | R150Y/R170E/E240N/T242V | 255 |
| D85N/K228N/R318Y/R338E/R403E/ | D[85]N/K63N/R150Y/R170E/R233E/ | 256 |
| E410N | E240N | |
| N260S/R318Y/R338E/R403E/E410N | N95S/R150Y/R170E/R233E/E240N | 257 |
| R318Y/R338E/N346D/R403E/E410N | R150Y/R170E/N178D/R233E/E240N | 258 |
| Y155F/N346D | Y[155]F/N178D | 259 |
| Y155F/R318Y/R338E/N346D/R403E/ | Y[155]F/R150Y/R170E/N178D/R233E/ | 260 |
| E410N | E240N | |
| Y155F/N260S/N346D/ | Y[155]F/N95S/N178D | 261 |
| K247N/N249S/N260S | K82N/N84S/N95S | 262 |
| D104N/K106S/N260S | D[104]N/K[106]S/N95S | 185 |
| Y155F/N260S | Y[155]F/N95S | 263 |
| K247N/N249S/N260S/R318Y/R338E/ | K82N/N84S/N95S/R150Y/R170E/R233E/E240N | 264 |
| R403E/E410N | | |
| D104N/K106S/N260S/R318Y/R338E/ | D[104]N/K[106]S/N95S/R150Y/ | 265 |
| R403E/E410N | R170E/R233E/E240N | |
| Y155F/N260S/R318Y/R338E/R403E/ | Y[155]F/N95S/R150Y/R170E/R233E/ | 266 |
| E410N | E240N | |
| R318Y/R338E/T343R/R403E/E410N | R150Y/R170E/T175R/R233E/E240N | 267 |
| R338E/T343R | R170E/T175R | 268 |
| D104N/K106S/Y155F/N260S | D[104]N/K[106]S/Y[155]F/N95S | 269 |
| Y155F/K247N/N249S/N260S | Y[155]F/K82N/N84S/N95S | 270 |
| D104N/K106S/K247N/N249S/N260S | D[104]N/K[106]S/K82N/N84S/N95S | 271 |
| D104N/K106S/Y155F/K247N/N249S/ | D[104]N/K[106]S/Y[155]F/ | 272 |
| N260S | K82N/N84S/N95S | |
| Y345A | Y177A | 213 |
| Y345T | Y177T | 214 |
| T343R | T175R | 209 |
| T343E | T175E | 210 |
| T343Q | T175Q | 211 |
| F342I | F174I | 212 |
| T343R/Y345T | T175R/Y177T | 215 |
| R318Y/R338E | R150Y/R170E | 188 |
| Y259F/K265T/Y345T | Y94F/K98T/Y177T | 216 |
| D104N/K106S/Y155F/K247N/N249S/ | D[104]N/K[106]S/Y[155]F/ | 326 |
| R318Y/R338E/R403E/E410N | K82N/N84S/R150Y/R170E/R233E/E240N | |
| D104N/K106S/K228N/K247N/N249S/ | D[104]N/K[106]S/K63N/K82N/ | 327 |
| R318Y/R338E/R403E/E410N | N84S/R150Y/R170E/R233E/E240N | |
| Y155F/K228N/K247N/N249S/R318Y/ | Y[155]F/K63N/K82N/N84S/R150Y/ | 328 |
| R338E/R403E/E410N | R170E/R233E/E240N | |
| Y155F/K247N/N249S/N260S/R318Y/ | Y[155]F/K82N/N84S/N95S/R150Y/ | 329 |
| R338E/R403E/E410N | R170E/R233E/E240N | |
| Y155F/R318Y/R338E/T343R/R403E/ | Y[155]F/R150Y/R170E/T175R/R233E/ | 330 |
| E410N | E240N | |
| D104N/K106S/R318Y/R338E/T343R/ | D[104]N/K[106]S/R150Y/ | 331 |
| R403E/E410N | R170E/T175R/R233E/E240N | |
| T343R/N346Y | T175R/N178Y | 332 |
| R318Y/R338E/N346Y/R403E/E410N | R150Y/R170E/N178Y/R233E/E240N | 333 |
| R318Y/R338E/T343R/N346Y/R403E/ | R150Y/R170E/T175R/N178Y/R233E/E240N | 334 |
| E410N | | |
| T343R/N346D | T175R/N178D | 335 |
| R318Y/R338E/T343R/N346D/R403E/ | R150Y/R170E/T175R/N178D/R233E/E240N | 336 |
| E410N | | |
| R318Y/R338E/Y345A/R403E/E410N | R150Y/R170E/Y177A/R233E/E240N | 337 |
| R318Y/R338E/Y345A/N346D/R403E/ | R150Y/R170E/Y177A/N178D/R233E/E240N | 338 |
| E410N | | |
| Y155F/K247N/N249S/R318Y/R338E/ | Y[155]F/K82N/N84S/R150Y/R170E/ | 339 |
| R403E | R233E | |
| K247N/N249S/R318Y/R338E/R403E | K82N/N84S/R150Y/R170E/R233E | 340 |
| Y155F/K247N/N249S/R318Y/R403E/ | Y[155]F/K82N/N84S/R150Y/R233E/ | 341 |
| E410N | E240N | |
| K247N/N249S/R318Y/R403E/E410N | K82N/N84S/R150Y/R233E/E240N | 342 |
| Y155F/K247N/N249S/R338E/R403E/ | Y[155]F/K82N/N84S/R170E/R233E/ | 343 |
| E410N | E240N | |
| K247N/N249S/R338E/R403E/E410N | K82N/N84S/R170E/R233E/E240N | 344 |
| R318Y/R338E/T343R/R403E | R150Y/R170E/T175R/R233E | 345 |
| Y155F/R318Y/R338E/T343R/R403E | Y[155]F/R150Y/R170E/T175R/R233E | 346 |
| R318Y/R338E/T343R/E410N | R150Y/R170E/T175R/E240N | 347 |
| Y155F/R318Y/R338E/T343R/E410N | Y[155]F/R150Y/R170E/T175R/E240N | 348 |
| R318Y/T343R/R403E/E410N | R150Y/T175R/R233E/E240N | 349 |
| Y155F/R318Y/T343R/R403E/E410N | Y[155]F/R150Y/T175R/R233E/E240N | 350 |
| R338E/T343R/R403E/E410N | R170E/T175R/R233E/E240N | 351 |
| Y155F/R338E/T343R/R403E/E410N | Y[155]F/R170E/T175R/R233E/E240N | 352 |
| Y155F/K247N/N249S/R318Y/R338E/ | Y[155]F/K82N/N84S/R150Y/R170E/ | 353 |
| T343R/R403E/E410N | T175R/R233E/E240N | |
| K247N/N249S/R318Y/R338E/T343R/ | K82N/N84S/R150Y/R170E/T175R/R233E/ | 354 |
| R403E/E410N | E240N | |
| K228N/I251S/R318Y/R338E/R403E/ | K63N/I86S/R150Y/R170E/R233E/E240N | 355 |
| E410N | | |
| Y155F/K228N/I251S/R318Y/R338E/ | Y[155]F/K63N/I86S/R150Y/R170E/ | 356 |
| R403E/E410N | R233E/E240N | |
| N260S/R318Y/R338E/T343R/R403E/ | N95S/R150Y/R170E/T175R/R233E/E240N | 357 |
| E410N | | |
| Y155F/N260S/R318Y/R338E/T343R/ | Y[155]F/N95S/R150Y/R170E/T175R/ | 358 |
| R403E/E410N | R233E/E240N | |
| K228N/K247N/N249S/R318Y/R338E/ | K63N/K82N/N84S/R150Y/R170E/T175R/ | 359 |
| T343R/R403E/E410N | R233E/E240N | |
| Y155F/K228N/K247N/N249S/R318Y/ | Y[155]F/K63N/K82N/N84S/R150Y/ | 360 |
| R338E/T343R/R403E/E410N | R170E/T175R/R233E/E240N | |
| Y155F/R338E/T343R/R403E | Y[155]F/R170E/T175R/R233E | 361 |
| R338E/T343R/R403E | R170E/T175R/R233E | 362 |
| Y155F/R338E/T343R/R403E/E410S | Y[155]F/R170E/T175R/R233E/E240S | 363 |
| Y155F/N260S/R338E/T343R/R403E | Y[155]F/N95S/R170E/T175R/R233E | 364 |
| Y155F/I251S/R338E/T343R/R403E | Y[155]F/I86S/R170E/T175R/R233E | 365 |
| R318Y/R338E/T343R/R403E/E410S | R150Y/R170E/T175R/R233E/E240S | 366 |
| Y155F/K247N/N249S/T343R/R403E | Y[155]F/K82N/N84S/T175R/R233E | 367 |
| Y155F/K247N/N249S/R318Y/R338E/ | Y[155]F/K82N/N84S/R150Y/R170E/ | 368 |
| T343R/R403E | T175R/R233E | |
| K247N/N249S/R318Y/R338E/T343R/ | K82N/N84S/R150Y/R170E/T175R/R233E | 369 |
| R403E | | |
| Y155F/K247N/N249S/R338E/T343R/ | Y[155]F/K82N/N84S/R170E/T175R/ | 370 |
| R403E/E410N | R233E/E240N | |
| K247N/N249S/R338E/T343R/R403E/ | K82N/N84S/R170E/T175R/R233E/E240N | 371 |
| E410N | | |
| Y155F/K247N/N249S/R318Y/R338E | Y[155]F/K82N/N84S/R150Y/R170E | 372 |
| Y155F/K247N/N249S/R318Y/T343R | Y[155]F/K82N/N84S/R150Y/T175R | 373 |
| Y155F/K247N/N249S/R318Y/R403E | Y[155]F/K82N/N84S/R150Y/R233E | 374 |
| Y155F/K247N/N249S/R318Y/E410N | Y[155]F/K82N/N84S/R150Y/E240N | 375 |
| Y155F/K247N/N249S/R338E/R403E | Y[155]F/K82N/N84S/R170E/R233E | 376 |
| Y155F/K247N/N249S/R338E/T343R | Y[155]F/K82N/N84S/R170E/T175R | 377 |
| Y155F/K247N/N249S/R318Y/R338E/ | Y[155]F/K82N/N84S/R150Y/R170E/ | 378 |
| T343R/E410N | T175R/E240N | |
| K247N/N249S/R318Y/R338E/T343R/ | K82N/N84S/R150Y/R170E/T175R/E240N | 379 |
| E410N | | |
| Y155F/K247N/N249S/R318Y/T343R/ | Y[155]F/K82N/N84S/R150Y/T175R/ | 380 |
| R403E/E410N | R233E/E240N | |
| K247N/N249S/R318Y/T343R/R403E/ | K82N/N84S/R150Y/T175R/R233E/E240N | 381 |
| E410N | | |
| Y155F/K247N/N249S/R338E/E410N | Y[155]F/K82N/N84S/R170E/E240N | 382 |
| Y155F/K247N/N249S/R318Y/T343R/ | Y[155]F/K82N/N84S/R150Y/T175R/ | 383 |
| R403E | R233E | |
| K247N/N249S/R318Y/T343R/R403E | K82N/N84S/R150Y/T175R/R233E | 384 |
| Y155F/K247N/N249S/R318Y/T343R/ | Y[155]F/K82N/N84S/R150Y/T175R/ | 385 |
| E410N | E240N | |
| K247N/N249S/R318Y/T343R/E410N | K82N/N84S/R150Y/T175R/E240N | 386 |
| Y155F/K247N/N249S/R338E/T343R/ | Y[155]F/K82N/N84S/R170E/T175R/ | 387 |
| R403E | R233E | |
| K247N/N249S/R338E/T343R/R403E | K82N/N84S/R170E/T175R/R233E | 388 |
| Y155F/K247N/N249S/R338E/T343R/ | Y[155]F/K82N/N84S/R170E/T175R/ | 389 |
| E410N | E240N | |
| K247N/N249S/R338E/T343R/E410N | K82N/N84S/R170E/T175R/E240N | 390 |
| Y155F/K247N/N249S/T343R/R403E/ | Y[155]F/K82N/N84S/T175R/R233E/ | 391 |
| E410N | E240N | |
| K247N/N249S/T343R/R403E/E410N | K82N/N84S/T175R/R233E/E240N | 392 |
| Y155F/R318Y/R338E/T343R | Y[155]F/R150Y/R170E/T175R | 393 |
| R318Y/R338E/T343R | R150Y/R170E/T175R | 394 |
| Y155F/R318Y/T343R/R403E | Y[155]F/R150Y/T175R/R233E | 395 |
| Y155F/T343R/R403E/E410N | Y[155]F/T175R/R233E/E240N | 396 |
| Y155F/K247N/N249S/R318Y/R338E/ | Y[155]F/K82N/N84S/R150Y/R170E/ | 397 |
| T343R | T175R | |
| K247N/N249S/R318Y/R338E/T343R | K82N/N84S/R150Y/R170E/T175R | 398 |
| Y155F/K247N/N249S/T343R/E410N | Y[155]F/K82N/N84S/T175R/E240N | 399 |
| Y155F/K247N/N249S/R403E/E410N | Y[155]F/K82N/N84S/R233E/E240N | 400 |
| Y155F/R338E/T343R/E410N | Y[155]F/R170E/T175R/E240N | 401 |
| R338E/T343R/E410N | R170E/T175R/E240N | 402 |
| Y155F/R318Y/T343R/E410N | Y[155]F/R150Y/T175R/E240N | 403 |
| R318Y/T343R/E410N | R150Y/T175R/E240N | 404 |
| K228N/R318Y/R338E/T343R/R403E/ | K63N/R150Y/R170E/T175R/R233E/E240N | 405 |
| E410N | | |
| K228N/K247N/N249S/R318Y/R338E/ | K63N/K82N/N84S/R150Y/R170E/T175R/ | 406 |
| T343R/R403E | R233E | |
| K228N/K247N/N249S/R318Y/R338E/ | K63N/K82N/N84S/R150Y/R170E/T175R/ | 407 |
| T343R/E410N | E240N | |
| K228N/K247N/N249S/R318Y/T343R/ | K63N/K82N/N84S/R150Y/T175R/R233E/ | 408 |
| R403E/E410N | E240N | |
| Y155F/R338E/R403E/E410N | Y[155]F/R170E/R233E/E240N | 409 |
| Y155F/R318Y/R338E/R403E | Y[155]F/R150Y/R170E/R233E | 410 |
| Y155F/R318Y/R403E/E410N | Y[155]F/R150Y/R233E/E240N | 411 |
| Y1N | Y[1]N | 412 |

### C. Expression and Purification of FIX Polypeptides

Wild-type and variant FIX polypeptides were expressed in CHO-Express (CHOX) cells (Excellgene). CHO Express (CHOX) cells were maintained in DM204B Complete medium (Irvine Scientific) and used to inoculate production seed cultures. Seed cultures were grown in the same media to approximately 1.4×10⁷ viable cells (vc)/mL and approximately 100 mL used to inoculate approximately 1.0 L of DM204B Complete media, so that the inoculation density was 1.2×10⁶ vc/mL. This culture was grown for 3 days to reach 13-16×10⁶ vc/mL on the day of transfection. A transfection complex was formed by mixing FIX plasmid DNA (3.2 mg) with Polyethylenimine “MAX” (PEI—20.5 mg (Polysciences)) and diluting to 1.0 L with serum-free TfMAX2 transfection medium (Mediatech). This mixture was then added to the 1.0 L production culture. 1.0 L aliquots of the cells plus transfection mix were split into 2×3 L baffled Fernback Flasks and allowed to express for 4 days before harvesting the crude FIX. Culture supernatants were then harvested by filtration and FIX was purified.

Larger-scale cultures of 10 L or greater were produced in WAVE bioreactors (GE Healthcare). 20 L wave bags were inoculated with approximately 400 mL of seed culture, grown as described above, with 4.6 L of DM204B Complete media to a seeding density of 1.2×10⁶ vc/mL. The WAVE bioreactor was set to a rocking angle of 6 degrees, rocking rate of 24 rpm at 37.1° C. in order to allow the cells to reach a cell density of 13-16×10⁶ vc/mL 3 days later. 16 mg of FIX plasmid DNA and 102.5 mg of PEI were combined to form a transfection complex, which was diluted in 5.0 L of TfMAX2 prior to addition to the culture on the WAVE bioreactor, 3 days after the initial seeding. While the Transfection complex plus TfMAX media was added to the wave bag, the rocking angle of the WAVE Bioreactor was set to 8 degrees and the temperature to 33° C., while the other settings remained the same. The culture was allowed to express for 4 days before harvesting the crude FIX. The contents of the wave bags were allowed to settle for 3 hrs at 4° C. prior to harvesting the culture supernatant through a CUNO depth filter and then the FIX was purified. FIX polypeptides were purified using a Capto Q column (GE Healthcare), to which FIX polypeptides with functional Gla domains adsorb, followed by a calcium elution step. Typically, EDTA (10 mM), Tris (25 mM, pH 8.0), and Tween-80 (0.001%) were added to the culture supernatant from the transfected cells. The samples were loaded onto a Capto Q column that had been pre-equilibrated with Buffer B (25 mM Tris pH 8, 1 M NaCl, 0.001% Tween-80), followed by equilibration with Buffer A (25 mM Tris pH 8, 0.15 M NaCl, 0.001% Tween-80). Immediately following completion of sample loading, the column was washed with 14% Buffer B (86% Buffer A) for 20 column volumes. Buffer C (25 mM Tris pH 8, 0.2 M NaCl, 0.001% Tween-80, 10 mM CaCl₂)) was then applied to the column to elute the FIX polypeptides that were collected as a pool.

The eluted pool was further purified using a Q Sepharose HP column (GE Healthcare). The sample was prepared for application by diluting with 2 volumes of Buffer D (25 mM Tris pH 8, 0.001% Tween-80). The diluted sample was loaded onto a Q Sepharose HP column that had been pre-equilibrated with Buffer F (25 mM Tris pH 8, 1 M NaCl, 2.5 mM CaCl₂), 0.001% Tween-80), followed by Buffer E (25 mM Tris pH 8, 2.5 mM CaCl₂), 0.001% Tween-80). After washing with 4% Buffer F (96% Buffer E), a gradient from 4-40% Buffer F was applied to the column and fractions were collected. Fractions containing FIX polypeptides were then pooled.

### D. Purification to Enrich for Glycosylated Polypeptides

The extent of glycosylation of the modified FIX polypeptides was estimated using SDS-polyacrylamide gel electrophoresis. Hyperglycosylation was assessed by comparison of the migration pattern of the modified FIX polypeptide with a wild type FIX, Benefix® Coagulation FIX. Hyperglycosylated forms of the enzyme migrated slower, exhibiting a higher apparent molecular weight, than the wild type polypeptide. It was observed that the polypeptides containing the E240N mutation, which introduces a non-native N-glycosylation site at position 240, were only partially glycosylated (approximately 20% glycosylation). To enrich for the hyperglycosylated form, a modification of the purification process described above was performed.

The first step of purification was performed using the Capto Q column, as described above. The eluted pool from this column was diluted with 2 volumes of Buffer D (as above) and the sample was loaded onto a Heparin Sepharose column that had been pre-equilibrated with Buffer F (as above), followed by Buffer E (as above). The column was then developed with a gradient from 0% to 70% Buffer F and fractions were collected. The hyperglycosylated form of the E410N variant eluted from the column in approximately 35% Buffer F, whereas the non-hyperglycosylated form eluted in approximately 50% Buffer F. Each collected pool was further purified on the Q Sepharose HP column as described above. By this method a pool containing approximately 80% hyperglycosylated form of the E410N variant was obtained. The extent of hyperglycosylation was estimated by visual inspection of SDS-polyacrylamide gel electrophoresis.

### E. Exemplary Modified FIX

An exemplary modified FIX polypeptide is that which contains the replacements R318Y/R338E/T343R (SEQ ID NO:394; referred to herein as CB2679d, and also, as ISU304 in the Examples below). It is a purified modified recombinant form of the human factor IX protein modified with three point mutations that is produced in Chinese Hamster Ovary (CHO) cells. FIX of SEQ ID NO:394, with the replacements R318Y/R338E/T343R, is a 415 amino acid glycoprotein. The primary amino acid sequence of CB 2679d, illustrated in FIG. 4 (and SEQ ID NO:394), where the mature protein has 415 amino acids and varies from natural functional human FIX by the introduction of 3 point mutations into 2 distinct, solvent exposed surface loops of the FIX protein. This variant has three mutations: R318Y/R338E/T343R. These mutations confer enhanced procoagulant activity and reduced elimination. FIG. 5 illustrates that structure and domains of the mature form of FIX that has three mutations: R318Y/R338E/T343R. Other modified FIX polypeptides, such as the FIX polypeptide contain R338E/T343R, which optionally can be PEGylated or albuminated or otherwise modified to increase serum half-life, described and provided herein that have enhanced activity and optionally increased serum half-life also can be used for prophylactic subcutaneous administration.

The modified FIX proteins can be provided, for example, as a lyophilized powder in vials containing approximately 8475 IU/mL presented in 1.4 mL/vial (total 11865 IU/vial) when reconstituted with sterile water for injection (SWFI). A smaller dosage size can be provided to facilitate treatment of infants and very young toddlers.

Structure-based rational design was used to endow the modified polypeptides provided herein with significantly enhanced procoagulant activity and increased duration of action compared with plasma-derived or recombinant, wild type FIX (WT-FIX) (e.g., FIX sold as BeneFIX®, Rixubis®, Refixia® (the brand name for nonacog beta pegol(N9-GP)), and recombinant extended half-life (EHL) Fc fusion protein FIX (e.g., Alprolix® FIX). The enhanced properties of the modified FIX of SEQ ID NO:394 result from the introduction of the three amino acid replacements: arginine (R) 318 replaced by tyrosine (Y)/arginine (R) 338 replaced with glutamic acid (E)/threonine (T) 343 replace by arginine (R). One mutation is located in the “150-loop,” also referred to as the “autolysis loop,” and two mutations are in the “170-loop.” The mutations in the “170-loop” act synergistically to significantly enhance the affinity of the protein for its co-factor FVIII, and may also stabilize the active conformation of FIXa. The mutation in the “150-loop” also stabilizes the active conformation of the FIXa structure and can interact directly with the substrate FX and the primary inhibitor anti-thrombin III (ATIII). Consequently, these mutations increase the procoagulant, catalytic efficiency of the FIX variant by a plurality of mechanisms, and also provide resistance to physiologically relevant inhibition and elimination. Other modified FIX provided herein that have enhanced activity (at least 7-10 fold compared to wild-type FIX) and optionally longer serum half-life or duration of action can be used in the methods and regimens herein. For example, a modified FIX with the replacements R338E/T343R and serum stability or half-life extender, such as by Fc fusion, albumination, PEGylation and other such modifications can be used in the methods herein.

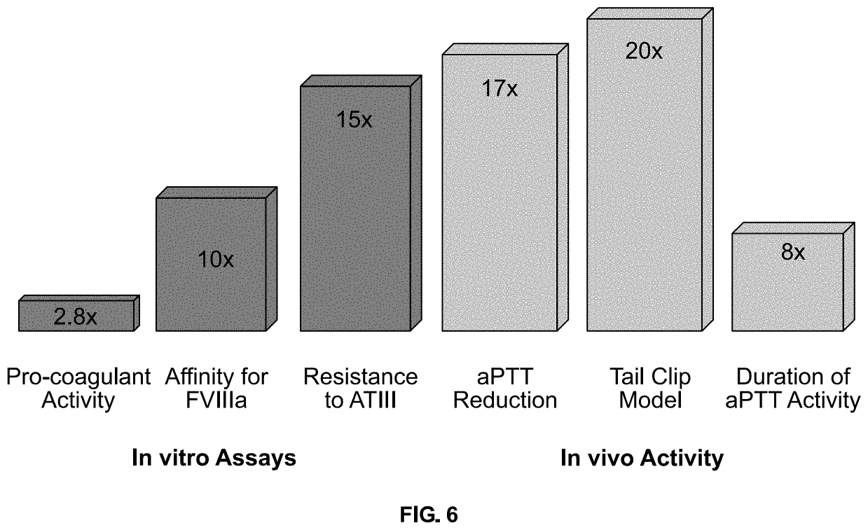

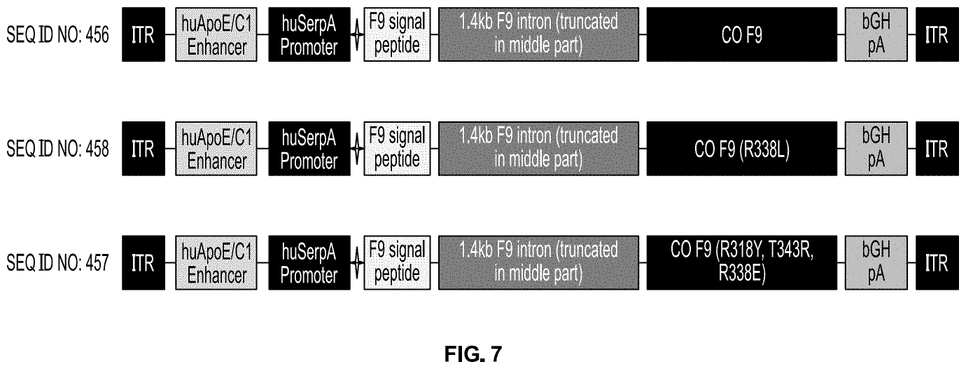

Due to these molecular alterations of its three-dimensional structure, compared with wild type FIX, the modified FIX of SEQ ID NO:394, with the replacements R318Y/R338E/T343R, exhibits in vitro approximately 3-fold enhanced catalytic efficiency for the activation of FX, 10-fold enhanced affinity for FVIIIa, and 15-fold resistance to inhibition by ATIII (Table 17 below and FIG. 6). These improved properties of CB 2679d were expected at the bleeding site to selectively accelerate the generation of the procoagulant activity (i.e., via FXa), and to increase the level and duration of its activity.

TABLE 17
Improved properties of FIX of SEQ ID NO: 394,
with the replacements R318Y/R338E/T343R

| | | Fold |
|---|---|---|
| | | Change |
| In Vitro | Pro-coagulant Activity | 2.8 |
| Assays | Affinity for FVIIIa | 10 |
| | Resistance to ATIII | 15 |
| In Vivo | aPTT reduction | 17 |
| Activity | Tail Clip Model | 20 |
| | Duration of aPTT Activity | 8 |

These properties were confirmed in vivo (see, e.g., FIG. 6); this modified FIX polypeptide displays 20-fold enhanced potency for inhibition of bleeding in a standard murine hemophilia tail cut model, a 17-fold reduction in (activated partial thromboplastin time) aPTT, and an 8-fold prolonged correction of aPTT activity compared with BeneFIX® FIX. Similar increased potency advantages of CB 2679d versus all commercially available FIX products are expected in man. In addition, significant improvements in duration of action versus BeneFIX® FIX, plasma-derived FIX (pdFIX), and other wild-type FIX products are expected when dosing is of equal mass/kg.

Activity Measurement

Function was measured in vitro using the one-stage clotting activity assay, which is one of the standardized tests for assessing the therapeutic potency of commercial factor IX preparations. WHO International Standard 4th International Standard for FIX Concentrate (NIBSC code 07/182) was used as a reference of Factor IX activity. Consistent with its greater potency in animal models, the specific activity of FIX of SEQ ID NO:394, with the replacements R318Y/R338E/T343R, in this assay is significantly higher (on average, 19-fold) than that of BeneFIX® FIX (see Table 18, below). The chromogenic assay also is used to measure potency and clinical activity in blood specimens (Table 19, below).

The one-stage clotting activity assay was performed using 3 batches of CB 2679d/ISU304 (batches B1528, B1531 and B1602Y); the average specific activity obtained from these 3 batches was compared with the specific activity of BeneFIX® FIX (batch J67791, a commercially available batch of BeneFIX® FIX). A standard curve was established using WHO standard in 1% BSA in TBS (pH 7.4).

TABLE 18
Specific Activity of the modified FIX of SEQ ID NO:
394 and BeneFIX ® Measured by One-Stage Clotting
Activity Assays Employing Different Type of Activators.

| | | FIX - SEQ ID | |
|---|---|---|---|
| | BeneFIX ® | NO: 394 (IU/mg) | Ratio of FIX of |
| | (IU/mg) | (B1528, B1531, | SEQ ID NO: 394 |
| Activator | (J67791) | B1602Y) | to BeneFIX ® |
| Stago PTT A | 262 | 5,705 | 22 |
| Pathromtin SL | 220 | 3,091 | 14 |
| Actin FS | 228 | 4,087 | 18 |
| C.K Prest^{†} | 257 | 5,101 | 20 |
| Mean ± SD | 242 ± 21 | 4,496 ± 1,150 | 19 |

^{†}C.K. Prest activator was employed in the release test of the modified FIX herein.

The modified FIX and BeneFIX® FIX potency were compared by clotting and Chromogenic assays (see Table 19, below).

TABLE 19
Summary of potency measurement by clotting and chromogenic assay

| | | | | Ratio of | | Ratio of |
|---|---|---|---|---|---|---|
| | | | Potency by | FIX of | | FIX of |
| | | | Clotting | SEQ ID | Potency by | SEQ ID |
| | | | assay, | NO: 394 to | Chromogenic | NO: 394 to |
| Name | Batch | mg/mL | IU/mg | BeneFIX ® | Assay, IU/mg | BeneFIX ® |
| BeneFIX ® | J67791 | 1.48 | 287 | — | 213 | — |
| FIX of SEQ | B1528 | 1.64 | 5,339 | 19 | 3.207 | 15 |
| ID NO: 394 | | | | | | |
| FIX of SEQ | E1601Y | 1.95 | 4,622 | 16 | 2.972 | 14 |
| ID NO: 394 | | | | | | |

These data show that the instantly provided modified FIX polypeptide is at least 14-fold more potent than BeneFIX® FIX in activity assays, with slightly lower activity when measured by chromogenic assay. A difference between one-stage clotting assay activity and chromogenic activity is commonly reported with modified recombinant FIX products. The variability of activity by varying activators is likewise well known. Thus, as described the instant polypeptides that contain the mutations R318Y/R338E/T343R have considerably enhanced potency compared to WT-FIX.

Functional Characterization the Modified FIX Polypeptides

Functional properties have been evaluated in a series of in vitro studies. The activation rate of the polypeptide of SEQ ID NO:394 (CB 2679d) by Factor XIa/calcium and the extent of activation (approximately 100%) were equivalent to those of commercial lots of BeneFIX® FIX. In contrast, the catalytic properties of fully activated CB 2679d under a variety of experimental conditions were improved compared with commercial lots of recombinant Factor IX preparations (EPAR), and native amino-acid sequence manufactured in the Sponsor's laboratory, referred to as ‘WT-recombinant’. The ability of activated FIX to bind to procoagulant phospholipid vesicles and activate factor X was improved over BeneFIX® FIX. Activated CB 2679d (FIX of SEQ ID NO:394) has a reduced rate of inhibition by antithrombin III (see Table 21 below). These improved properties mediate the significantly enhanced procoagulant potency of CB 2679d, as indicated by its specific activity of 3,091-5,705 IU/mg, compared with the reported and observed range of 220 to 262 IU/mg for BeneFIX® FIX.

The catalytic properties of fully activated CB 2679d under a variety of experimental conditions are improved compared with competing recombinant forms. The activated preparation of variant FIX (FIX of SEQ ID NO:394; CB 2679d) catalyzes the proteolytic cleavage and activation of purified factor X to the same extent in the presence of (1) poly-L-lysine phospholipid/calcium, and (3) factor VIIIa/phospholipid/calcium.

TABLE 20
Kinetic Analysis of FX Activation

| | Cofactor-Dependent | Fold Increase |
|---|---|---|
| | Indirect kcat/KM | over BeneFIX ® |
| Variant | (M-1s-1) | FIX |
| BeneFIX ® FIX | 4.3E+07 | 1 |
| WT-recombinant | 4.6E+07 | 1.1 |
| FIX of SEQ ID NO: 394 | 1.2E+08 | 2.8 |

The modified FIX provided herein has 2.8-fold higher cofactor dependent activity than BeneFIX® FIX (see Table 20).

TABLE 21
ATIII Inhibition of FIXa variants

| | ATIII Kapp | Fold |
|---|---|---|
| Variant | (M-1s-1) | decrease |
| BeneFIX ® FIX | 1.6E+07 | 1.4 |
| WT-recombinant | 2.4E+07 | 1 |
| FIX of SEQ ID NO: 394 | 1.1E+06 | 21.8 |

The modified FIX of SEQ ID NO:394 herein has ˜16-fold higher resistance to ATIII than BeneFIX® FIX.

The modified FIX (FIX of SEQ ID NO:394; CB 2679d/ISU304) has a high affinity to FVIIIa. In a situation where FVIIIa is absent (e.g., in the circulation), it is no longer active until FVIIIa is generated, so a prothrombotic risk is not present. Data presented in Table 22 below illustrate the effect of FVIIIa on the FIX of SEQ ID NO:394 (CB 2679d/ISU304) and BeneFIX® FIX. In the absence of FVIIIa, CB 2679d/ISU304 is 1.3-1.4-fold more active than BeneFIX® FIX; but its activity increases up to 486-fold when FVIIIa was added, while BeneFIX® FIX activity increases only up to 239-fold.

TABLE 22
Effect of FVIIIa concentration on FIX activity.

| | Test # | | | | | | | | |
|---|---|---|---|---|---|---|---|---|---|
| | 1 | | | 2 | | | 3 | | |
| | | | Fold- | | | Fold- | | | Fold- |
| | | | increase | | | increase | | | increase |
| | | | after | | | after | | | after |
| FVIIIa | | | FVIIIa | | | FVIIIa | | | FVIIIa |
| (ng/mL) | 0 | 5 | addition | 0 | 10 | addition | 0 | 15 | addition |
| CB2679d | | | | | | | | | |
| Mean | 1.24 | 278 | 225 | 1.25 | 479 | 384 | 1.28 | 622 | 486 |
| SD | 0.01 | 15 | | 0.00 | 28 | | 0.01 | 16 | |
| BeneFIX ® | | | | | | | | | |
| FIX | | | | | | | | | |
| Mean | 0.95 | 142 | 115 | 0.88 | 235 | 188 | 1.01 | 308 | 239 |
| SD | 0.01 | 9 | | 0.01 | 14 | | 0.00 | 3 | |
| Ratio | 1.3 | 2.0 | | 1.4 | 2.0 | | 1.3 | 2.0 | |
| (CB2679d vs | | | | | | | | | |
| BeneFIX ® | | | | | | | | | |
| FIX) | | | | | | | | | |

Measured in the presence of phospholipid vesicles (75% phosphatidyl-choline:25% phosphatidyl-serine).

Hemostatic activity was compared to the plasma-derived and recombinant products in a series of experiments that measured the thrombin generation potential of the preparations. The modified FIX provided herein shortens thrombin generation lag time and increases the amount of thrombin formed beyond that observed for BeneFIX® FIX. This indicates that its thrombogenic potential is greater than that of BeneFIX® FIX in individuals with hemophilia.

Hence, these results show that the catalytic properties, thrombin generation potential and hemostatic properties of modified FIX polypeptides provided herein are improved compared to those of recombinant and plasma-derived Factor IX products. These effects and resulting increased potency, unlike other available FIX products, renders such modified FIX polypeptides advantageous for gene therapy to provide sufficient FIX activity to effect prophylactic treatment of hemophilia such that treated subjects have normal or near normal clotting activity.

### Example 2

### Activation of FX and Determination of the Catalytically Active Protease (FXa) Concentration Using the Active Site Titrant Fluorescein-Mono-p′-Guanidinobenzoate (FMGB)

The concentration of Factor X (FX) in a stock of FX that can become catalytically active was determined. This stock of FX was then used in subsequent studies to calculate the catalytic activity of FIX variants for FX. Following activation of FX to FXa, the active site titration assay was carried out essentially as described by Bock et al. (Archives of Biochemistry and Biophysics (1989) 273:375-388) using the fluorogenic ester substrate fluorescein-mono-p′-guanidinobenzoate (FMGB), with a few minor modifications. FMGB readily reacts with FXa, but not FX or inactive protease, to form an effectively stable acyl-enzyme intermediate under conditions in which the concentration of FMGB is saturating and deacylation is especially slow and rate limiting for catalysis. Under these conditions, the FXa protease undergoes a single catalytic turnover to release the fluorescein fluorophore. When the initial burst of fluorescence is calibrated to an external concentration standard curve of fluorescein fluorescence, the concentration of active sites can be calculated.

### A. Activation of FX to FXa

The concentration of FX in a stock solution that is able to become catalytically active was determined by activation of FX samples with Russell's Viper Venom, followed by titrating the active FX (FXa) with FMGB. FX zymogen stocks were first pre-treated by the supplier with DFP (diisopropylfluorophosphate) and EGR-cmk to reduce the background FXa activity. FXa activation reactions were prepared with a final concentration of 10 μM FX (based on the A₂₈₀ absorbance and an extinction coefficient of 1.16) in a final volume of 50-100 μL in a reaction buffer containing 100 mM Tris, 50 mM NaCl, 5 mM CaCl₂), 0.1% PEG 8000, pH 8.1. Activation was initiated by the addition of Russell's Viper Venom (RVV-Xase; Heamatologic Technologies, Inc.) to a final concentration of 5 μg/mL (5 μL of a 98 μg/mL dilution per 100 μL reaction or 2.5 μL per 50 μL reaction) at 37° C. for 45-60 min of activation time (previously determined to represent complete activation by collecting samples every 15 min and testing the increase in cleavage of Spectrafluor FXa fluorogenic substrate). Reactions were quenched with 1/10 volume of quench buffer containing 100 mM Tris, 50 mM NaCl, 5 mM, 100 mM EDTA, 0.1% PEG 8000, pH 8.1.

### B. Active Site Titration

The active site titration assays were performed with a 1 mL reaction volume in a 0.4 cm×1 cm quartz cuvette under continuous stirring. Reactions contained 100-400 nM of the freshly activated FXa and 5 μM FMGB in an assay buffer containing 30 mM Hepes, 135 mM NaCl, 1 mM EDTA and 0.1% PEG 8000, pH 7.4. FMGB was prepared at a stock concentration of 0.01 M in DMF based on the dry weight and the concentration confirmed by absorbance spectroscopy at 452 nm using an extinction coefficient of 19,498 M⁻¹ cm⁻¹ in Phosphate Buffered Saline (PBS), pH 7.2. Assays were initiated by adding 5 μL of 1 mM FMGB (5 μM final concentration) to 1 mL of 1× assay buffer and first measuring the background hydrolysis of FMGB for ˜150-200 seconds before the addition of FXa to a final concentration of ˜100-400 nM. The release of fluorescein fluorescence in the burst phase of the reaction was followed for an additional 3600 seconds.

The amount of fluorescein released following catalysis of FMGB by FXa was determined using a standard curve of free fluorescein. The fluorescein standard solution was freshly prepared at a stock concentration of ˜70-150 mM in DMF and the accurate concentration was confirmed by absorbance spectroscopy under standard conditions at 496 nm using an extinction coefficient of 89,125 M⁻¹ cm⁻¹ in 0.1 N NaOH. A standard curve of free fluorescein was then prepared by titration of the absorbance-calibrated fluorescein standard into 1× assay buffer in 20 nM steps to a final concentration of 260-300 nM.

For data analysis, reaction traces were imported into the Graphpad Prism software package and the contribution of background hydrolysis was subtracted from the curve by extrapolation of the initial measured rate of spontaneous FMGB hydrolysis, which was typically less than 5% of the total fluorescence burst. The corrected curve was fit to a single exponential equation with a linear component (to account for the slow rate of deacylation) of the form ΔFluorescence=Amp(1−e^{-kt})+Bt, where Amp=the amplitude of the burst phase under the saturating assay conditions outline above, k is the observed first order rate constant for acyl-enzyme formation and B is a bulk rate constant associated with complete turnover of FMGB. The concentration of active FXa protease was calculated by comparison of the fit parameter for amplitude to the fluorescein standard curve. The values from multiple assays were measured, averaged and the standard deviation determined. The amount of active FXa in the preparation directly represents the concentration of FX in a stock preparation that can be activated by FIXa. This active site titrated value was employed when calculating the concentration of FX to be used in an indirect assay, such as the cofactor-dependent assay described in Example 4, below.

### Example 3

### Activation of FIX and Determination of the Catalytically Active Protease (FIXa) Concentration Using the Active Site Titrant 4-methylumbelliferyl p′-Guanidinobenzoate (MUGB)

The concentration of Factor IX (FIX) in a stock solution of the FIX zymogen that is able to become catalytically active was determined by activation of FIX samples, including FIX variants, with Factor XIa (FXIa; Heamatologic Technologies, Inc.) followed by titrating the active Factor IX (FIXa) with 4-methylumbelliferyl p′-guanidinobenzoate (MUGB).

### A. Activation of FIX to FIXa

Total protein concentrations in the FIX polypeptide preparations were determined by the A₂₈₀ absorbance using an extinction coefficient unique for each variant (i.e. ε₂₈₀=number of Tyr residues×1490+number Trp residues×5500+number Cys residues×125). Activation reactions of FIX to FIXa were prepared at a final concentration of 10 μM FIX in a final volume of 200-500 μL in a reaction buffer containing 100 mM Tris, 50 mM NaCl, 5 mM CaCl₂), 0.1% PEG 8000, pH 8.1. Activations were initiated by the addition of FXIa or biotinylated FXIa to a final concentration of 20 nM at 37° C. for 60 min of activation time. A 60 minute activation time was previously determined to represent complete activation by collecting samples every 15 min and assaying for total cleavage by SDS-PAGE.

The free FXIa or biotinylated FXIa used in the activation reaction was then removed from the samples using one of two methods that produce equivalent results, each removing greater than 95-97% of the catalytic FXIa. In the first method, which was used to remove free FXIa, activation reactions initiated with FXIa were mixed with an anti-FXIa monoclonal antibody (Abcam 20377) to a final concentration of 50 nM for 60 min at 37° C. Antibody capture of free FXIa was followed by the addition of washed protein G Dynal Beads (30 mg/mL; Invitrogen) to a final concentration of 25% vol:vol for an additional 120 min at room temperature. The Dynal Beads were removed from the solution per the manufacturer's instructions. In the second method, which was used to removed biotinylated FXIa, activation reactions using biotinylated FXIa were mixed with Streptavidin Dynal Beads (10 mg/mL; Invitrogen) to a final concentration of 10% vol:vol for 60 min at room temperature. The Dynal Beads were then removed per the manufacturer's instructions. Following removal of the FXIa, the total protein concentrations of activated FIXa samples were determined by A₂₈₀ absorbance using an extinction coefficient unique for each variant (as described above).

### B. Active Site Titration of FIXa

The concentration of catalytically active FIXa in an activated stock solution was determined by titrating the FIXa samples with a fluorogenic ester substrate, 4-methylumbelliferyl p′-guanidinobenzoate (MUGB). The principle titration assay was carried out essentially as described by Payne et al. (Biochemistry (1996) 35:7100-7106) with a few minor modifications to account for the slower reactivity of MUGB with FIXa. MUGB readily reacts with FIXa, but not FIX or inactive protease, to form an effectively stable acyl-enzyme intermediate under conditions in which the concentration of MUGB is saturating and deacylation is especially slow and rate limiting for catalysis. Under these conditions, the FIXa protease undergoes a single catalytic turnover to release the 4-methylumbelliferone fluorophore (4-MU). When the initial burst of fluorescence is calibrated to an external concentration standard curve of 4-MU fluorescence, the concentration of active sites can be calculated.

Assays were performed with a 1 mL reaction volume in a 0.4 cm×1 cm quartz cuvette, under continuous stirring with an assay buffer containing 50 mM Hepes, 100 mM NaCl, 5 mM CaCl₂) and 0.1% PEG 8000, pH 7.6. MUGB was prepared at a stock concentration of 0.04 M in DMSO based on the dry weight and diluted to a working concentration of 2 mM in DMSO. Titration assays were initiated by adding 4 μL of 2 mM MUGB to a final concentration of 8 μM in 1× assay buffer and first measuring the background hydrolysis of MUGB for ˜200-300 seconds before the addition of the FIXa or FIXa variant to a final concentration of 100-200 nM based on the total protein concentration determined for the activation reaction after removal of FXIa. The release of 4-MU fluorescence in the burst phase of the reaction was followed for a total of 2 hours in order to acquire sufficient data from the initial burst and subsequent steady state phases.

The amount of 4-MU released following catalysis of MUGB by FIXa was determined using a standard curve of 4-MU. A 4-MU standard solution was prepared at a stock concentration of 0.5 M in DMSO and the concentration confirmed by absorbance spectroscopy at 360 nm using an extinction coefficient of 19,000 M⁻¹ cm⁻¹ in 50 mM Tris buffer, pH 9.0. The standard curve of free 4-MU was prepared by titration of the absorbance-calibrated 4-MU into 1× assay buffer in 20 nM steps to a final concentration of 260-300 nM 4-MU.

For data analysis, reaction traces were imported into the Graphpad Prism software package and the contribution of background hydrolysis was subtracted from the curve by extrapolation of the initial measured rate of spontaneous MUGB hydrolysis, which was typically less than 5% of the total fluorescence burst. The corrected curve was fit to a single exponential equation with a linear component (to account for the slow rate of deacylation in the steady state phase) of the form ΔFluorescence=Amp(1−e^{-kt})+Bt, where Amp=the amplitude of the burst phase under the saturating assay conditions outline above, k is the observed first order rate constant for acyl-enzyme formation and B is a bulk rate constant associated with complete turnover of MUGB. The concentration of active FIXa protease is calculated by comparison of the fit parameter for amplitude to the 4-MU standard curve. The values from multiple assays were measured, averaged and the standard deviation determined. The concentration of FIX zymogen, which may become activated, in a stock solution was then determined by multiplying the A₂₈₀ determined total concentration of the FIX zymogen by the experimentally determined fraction active value for the fully activated sample (concentration of active FIXa/total concentration of FIXa).

### Example 4

### Determination of the Catalytic Activity of FIXa for its Substrate, Factor X

The catalytic activity of the FIXa variants for the substrate, Factor X (FX), was assessed indirectly in a fluorogenic assay by assaying for the activity of FXa, generated upon activation by FIXa, on the synthetic substrate Spectrafluor FXa. A range of FX concentrations were used to calculate the kinetic rate constants where the substrate protease (FX) was in excess by at least a 1000-fold over the concentration of the activating protease (FIXa). Briefly, activated and active site titrated FIXa was incubated in a calcium containing buffer with recombinant FVIII, phospholipid vesicles and alpha-thrombin (to activate FVIII to FVIIIa), forming the tenase (Xase) complex. The activity of alpha-thrombin was then quenched by the addition of a highly specific thrombin inhibitor, hirudin, prior to initiating the assay. FIXa variants (as part of the Xase complex) were subsequently mixed with various concentrations of FX and the fluorescent substrate, Spectrafluor FXa (CH₃SO₂-D-CHA-Gly-Arg-AMC) to initiate the assay. The release of the free fluorophore, AMC (7-amino-4-methylcoumarin) following catalysis of Spectrafluor FXa by FXa was then assessed continuously over a time period, and the kinetic rate constants of the FIXa variants determined.

### A. Assay Protocol

For assays evaluating the kinetic rate of FX activation by FIXa in the presence of FVIIIa and phospholipids, recombinant FVIII (Kogenate FS®; Bayer healthcare) was first resuspended in 5 mL of the provided diluent according to the manufacturer's instructions. The molar concentration of FVIII was then determined by absorbance at 280 nm using an extinction coefficient of 1.567 mg⁻¹ mL cm⁻¹ and a molecular weight of 163.6 kDa. The FIX variants were expressed, purified, activated and active site titrated as described in Examples 1-3, above. FIXa variants were then serially diluted to a concentration of 16 pM in a 200 μL volume of 1× Buffer A (20 mM Hepes/150 mM NaCl/5 mM CaCl₂)/0.1% BSA/0.1% PEG-8000, pH 7.4). In preparation for activation of FVIII to FVIIIa in the presence of FIXa and phospholipids, alpha-thrombin (Heamatologic Technologies, Inc.) and hirudin (American Diagnostica) were each diluted in a 1.0 mL volume of 1× Buffer A to 64 nM and 640 nM, respectively. Reconstituted FVIII was further diluted to a concentration of 267 nM in a 10 mL volume of 1× Buffer A containing 267 μM freshly resuspended phospholipids (75% phosphatidylcholine (PC)/25% phosphatidylserine (PS); PS/PC vesicles˜120 nm in diameter; Avanti Polar Lipids). FVIII was activated to FVIIIa by mixing 600 μL of the above FVIII/PC/PS solution with 100 μL of the 16 pM wild-type FIXa or FIXa variant dilution and 50 μL of the 64 nM alpha-thrombin solution followed by 15 minutes of incubation at 25° C. Activation reactions were subsequently quenched by the addition of 50 μL of the above 640 nM hirudin solution for 5 min at 25° C. prior to initiating the kinetic assay for FX activation. The final concentration of reagents in the 800 μL Xase complex solutions was as follows: 2 pM FIXa variant, 200 nM FVIIIa, 200 μM PC/PS vesicles, 4 nM alpha-thrombin (inhibited) and 40 nM hirudin.

A total of 25 μL of each Xase complex solution (FIXa/FVIIIa/Phospholipids/Ca²⁺) was aliquoted into a 96-well half-area black assay plate according to a predefined plate map (4 FIXa variants/plate). A solution of 900 nM active site titrated and DFP/EGR-cmk treated FX (see Example 2, above) was prepared in 5.6 mL of 1× Buffer A containing 1.0 mM Spectrafluor Xa substrate. This represented the highest concentration of FX tested and a sufficient volume for 4 assays. The FX/Spectrafluor Xa solution was then serially diluted 1.8-fold in an 8-channel deep-well polypropylene plate with a final volume of 2.5 mL 1× Buffer A that contains 1.0 mM Spectrafluor Xa, resulting in final dilutions of 900 nM, 500 nM, 277.8 nM, 154.3 nM, 85.7 nM, 47.6 nM, 25.6 nM and 0 nM FX. Alternatively in some assays, the FX/Specrafluor Xa solution was then serially diluted 1.5-fold in a 12-channel deep-well polypropylene plate with a final volume of 2.5 mL 1× Buffer A that contains 1.0 mM Spectrafluor Xa, resulting in final dilutions of 900 nM, 600 nM, 400 nM, 266.7 nM, 177.8 nM, 118.5 nM, 79.0 nM, 52.7 nM, 35.1 nM, 23.4 nM, 15.6 nM and 0 nM FX. Assay reactions were typically initiated using a BioMek FX liquid handling system programmed to dispense 25 μL of the FX/Spectrafluor Xa dilutions into 4 assay plates containing 25 μL of each FIXa variant (Xase complex). The final concentrations of the reagents in the assay were as follows: 1 pM FIXa, 100 nM FVIIIa, 100 μM PC/PS vesicles, 0.5 mM Spectrafluor Xa, 2 nM alpha-thrombin (inhibited), 20 nM hirudin and FX dilutions of 0 nM to 450 nM. Reactions were monitored in a SpectraMax fluorescence plate reader for 30 min at 37° C. A standard curve of free AMC served as the conversion factor for RFU to μM in the subsequent data analysis calculations using a dose range that covered 0 μM to 100 μM AMC.

### B. Data Analysis

All equations used to determine the steady-state kinetics of the catalysis of FX by FIXa are based on those described in the reference “Zymogen-Activation Kinetics: Modulatory effects of trans-4-(aminomethyl)cyclohexane-1-carboxylic acid and poly-D-lysine on plasminogen activation” in Petersen, et al. (1985) Biochem. J. 225:149-158. The theory for the steady-state kinetics of the system described by Scheme A (see below) is described by the expression of equation (1) that represents a parabolic accumulation of product.

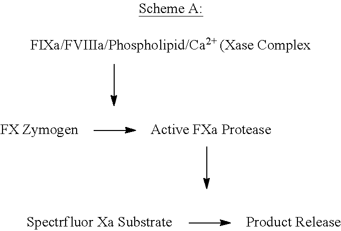

According to the mechanism of Scheme A, a₀ is the concentration of activating protease (FIXa), z₀ is the concentration of zymogen (FX), kₐ and K_{z} represent the k_{cat} and K_{M} for the activator-catalyzed conversion of zymogen to active enzyme (FXa), whereas kₑ and Kₛ represent the k_{cat} and K_{M} for conversion of substrate to product by FXa over a given time t:

$$p = {a}_{0} \frac{{k}_{a} [ {z}_{0} ]}{{K}_{z} + [ {z}_{0} ]} * \frac{{k}_{e} [ {S}_{0} ]}{{K}_{s} + [ {S}_{0} ]} * \frac{{t}^{2}}{2} \qquad Equation ( 1 )$$

For analysis of progress curves, equation (1) was re-cast in the form of equation (2) where the steady-state kinetics of FXa hydrolysis of the fluorogenic substrate were determined independently and replaced by the compound constant k₂.

$$p = {a}_{0} \frac{{k}_{a} [ {z}_{0} ]}{{K}_{z} + [ {z}_{0} ]} * {k}_{2} * \frac{{t}^{2}}{2} \qquad Equation ( 2 )$$

The FXa activity on Spectrofluor FXa in 1× Buffer A was independently determined to have a K_{M} of 313.0 μM and a k_{cat} value of 146.4 s⁻¹. Substitution of these values into equation (3) gave a k₂ correction factor of 90 s⁻¹.

$${k}_{2} = \frac{{k}_{e} [ {S}_{0} ]}{{K}_{M} + [ {S}_{0} ]} \qquad Equation ( 3 )$$

To determine the degree of FIXa catalytic activity, raw data collected with the SoftMax Pro application (Molecular Devices) were exported as .XML files or .TXT files. Further non-linear data analyses were performed with XLfit4, a software package for automated curve fitting and statistical analysis within the Microsoft Excel spreadsheet environment (IDBS Software) or directly within the ActivityBase software package using the XE Runner data analysis module (IDBS Software). The spreadsheet template was set up to automatically fit the parabolic reaction velocities (μM/sec²) of the tested FIXa variants at each FX concentration to the function of a standard rectangular hyperbola (i.e. Michaelis Menten equation) given by equation (4) to yield the fit values for Vₘₐₓ and K_{M}.

$$Reaction Velocity ( µ M / {sec}^{2} ) = \frac{{V}_{max} [ {S}_{0} ]}{{K}_{M} + [ {S}_{0} ]} \qquad Equation ( 4 )$$

The k_{cat} value for the tested FIXa variant was then calculated from the fit value for Vₘₐₓ (μM/sec²) by equation (5).

$${k}_{cat} = \frac{{V}_{max}}{[ FIXa ] * 0.5 * {k}_{2}} \qquad Equation ( 5 )$$

The specificity constant k_{cat}/K_{M} was calculated directly from the fit value of K_{M} and the calculated k_{cat} that arose from evaluation of equation (5) above.

Tables 23-28 set forth the catalytic activity for each of the FIXa variants assayed. Also assayed were recombinant wild-type FIXa (termed Catalyst Biosciences WT; generated as described above in Example 1), plasma purified FIXa (Haematologic Technologies, Inc.), and BeneFIX® (Coagulation Factor IX (Recombinant); Wyeth). Tables 23-24 present the results expressed as the kinetic constant for catalytic activity, k_{cat}/K_{M} (M⁻¹s⁻¹), and also as the percentage of the activity of the wild-type FIXa, wherein the activity is catalytic activity, k_{cat}/K_{M} (M⁻¹ s⁻¹) of each FIXa variant for its substrate, FX. The individual rate constants k_{cat} and K_{M} are provided in Tables 25 and 26, and 27 and 28, respectively. Tables 24, 26 and 28 reflect data for additional FIXa variants and provide new overall averages calculated to include additional experimental replicates (n) for FIXa variants in Tables 23, 25 and 27. Where the activity of the FIXa variant was compared to wild-type FIXa, it was compared to a recombinant wild-type FIXa polypeptide that was expressed and purified using the same conditions as used for the variant FIXa polypeptides to ensure that any differences in activity were the result of the mutation(s), and not the result of differences in, for example, post-translational modifications associated with different expression systems. Thus, the wild-type FIXa polypeptide used for comparison was the recombinant wild-type FIXa generated from cloning the FIX gene set forth in SEQ ID NO:1 and expressed from CHOX cells as a polypeptide with an amino acid sequence set forth in SEQ ID NO:3, as described in Example 1 (i.e., Catalyst Biosciences WT FIX polypeptide). The standard deviation (S.D.), coefficient of variation (as a percentage; % CV) and the number of assays performed (n) also are provided for each kinetic parameter.

The observed catalytic activities of the FIXa variants ranged from no detectable Xase activity in a few variants (e.g., FIXa-F314N/H315S, FIXa-G317N, FIXa-R318N/A320S and FIXa-K400E/R403E) to a greater than 10-fold increase in k_{cat}/K_{M} for the activation of FX compared to wild-type FIXa. Some of the variants displayed markedly increased catalytic activity compared to the wild-type FIXa, including FIXa-R338E, FIXa-R338A, FIXa-T343R, FIXa-E410N and combinations thereof such as FIXa-R318Y/R338E/E410N, FIXa-R318Y/R338E/R402E/E410N, FIXa-R318Y/R338E/T343R/R402E/E410N, FIXa-R318Y/R338E/T343R/E410N and FIXa-R338E/T343R displayed some of the greatest increases in catalytic activity.

Although several FIXa variants with single or multiple additional glycosylation sites demonstrated close to wild-type activity (e.g., FIXa-I251S, FIXa-D85N/I251S, FIXa-K63N, FIXa-K247N/N249S, and FIXa-K63N/K247N/N249S) or improved activity when combined with other mutations (e.g., FIXa-K247N/N249S/R338E/T343R/R403E and FIXa-K247N/N249S/R318Y/R338E/T343R/R403E/E410N), others showed reduced catalytic activity. The augmented catalytic activity was due to improvements in k_{cat} or K_{M} or most often, both parameters.

TABLE 23
Catalytic activity of FIXa variants (k_{cat}/K_{M})

| | Mutation | | | | % of | |
|---|---|---|---|---|---|---|
| Mutation (Mature | (Chymotrypsin | k_{cat}/K_{M} | ±S.D. | | WT | |
| FIX Numbering) | Numbering) | (M⁻¹s⁻¹) | (M⁻¹V⁻¹) | % CV | k_{cat}/K_{M} | n |
| BeneFIX ® | BeneFIX ® | 4.1E+07 | 2.1E+07 | 51% | 91% | 125 |
| Coagulation FIX | Coagulation FIX | | | | | |
| (T148A) | (T[148]A) | | | | | |
| Plasma Purified | Plasma Purified | 5.2E+07 | 2.2E+07 | 41% | 117% | 120 |
| FIXa | FIXa | | | | | |
| Catalyst Biosciences | Catalyst Biosciences | 4.5E+07 | 2.5E+07 | 56% | 100% | 31 |
| WT | WT | | | | | |
| N157D | N[157]D | 2.9E+07 | 8.1E+06 | 28% | 64% | 2 |
| Y155F | Y[155]F | 4.1E+07 | 1.3E+05 | 0% | 93% | 2 |
| A103N/N105S/ | A[103]N/N[105]S/ | 3.9E+07 | 1.4E+06 | 4% | 88% | 2 |
| Y155F | Y[155]F | | | | | |
| D104N/K106S/ | D[104]N/K[106]S/ | 3.6E+07 | 1.0E+06 | 3% | 81% | 2 |
| Y155F | Y[155]F | | | | | |
| A103N/N105S | A[103]N/N[105]S | 3.7E+07 | 1.4E+07 | 38% | 82% | 9 |
| D104N/K106S | D[104]N/K[106]S | 3.8E+07 | 1.3E+07 | 34% | 86% | 9 |
| K106N/V108S | K[106]N/V[108]S | 2.8E+07 | 6.7E+06 | 24% | 62% | 7 |
| D85N | D[85]N | 7.3E+07 | 2.8E+07 | 38% | 164% | 15 |
| T148A | T[148]A | 4.0E+07 | 2.5E+07 | 62% | 89% | 30 |
| T148A† | T[148]A† | 2.3E+07 | 7.6E+06 | 33% | 52% | 7 |
| K5A | K[5]A | 5.6E+07 | 4.5E+06 | 8% | 125% | 2 |
| D64N | D[64]N | 1.0E+07 | 1.9E+06 | 19% | 22% | 2 |
| D64A | D[64]A | 2.5E+06 | 1.1E+06 | 47% | 5% | 2 |
| N167D | N[167]D | 3.1E+07 | 1.1E+07 | 34% | 69% | 2 |
| N167Q | N[167]Q | 3.5E+07 | 1.9E+07 | 53% | 79% | 4 |
| S61A | S[61]A | 4.8E+07 | 2.5E+07 | 52% | 108% | 4 |
| S53A | S[53]A | 3.5E+07 | 1.7E+07 | 48% | 78% | 3 |
| T159A | T[159]A | 3.7E+07 | 1.2E+07 | 33% | 82% | 3 |
| T169A | T[169]A | 4.7E+07 | 2.0E+07 | 43% | 106% | 3 |
| T172A | T[172]A | 5.0E+07 | 2.6E+07 | 52% | 112% | 3 |
| T179A | T[179]A | 5.5E+07 | 1.3E+07 | 23% | 122% | 3 |
| Y155H | Y[155]H | 5.0E+07 | 1.4E+07 | 27% | 113% | 3 |
| Y155Q | Y[155]Q | 5.4E+07 | 2.0E+07 | 36% | 121% | 3 |
| S158A | S[158]A | 3.6E+07 | 1.1E+06 | 3% | 81% | 2 |
| S158D | S[158]D | 4.0E+07 | 9.3E+05 | 2% | 89% | 2 |
| S158E | S[158]E | 3.7E+07 | 3.5E+06 | 9% | 82% | 2 |
| N157Q | N[157]Q | 3.2E+07 | 2.8E+06 | 9% | 72% | 2 |
| D203N/F205T | D39N/F41T | 2.2E+07 | 1.2E+07 | 53% | 50% | 12 |
| D85N/D203N/ | D[85]N/D39N/F41T | 3.0E+07 | 6.4E+06 | 22% | 66% | 5 |
| F205T | | | | | | |
| K228N | K63N | 3.6E+07 | 1.7E+07 | 49% | 80% | 13 |
| D85N/K228N | D[85]N/K63N | 4.6E+07 | 1.5E+07 | 32% | 104% | 6 |
| A103N/N105S/ | A[103]N/N[105]S/ | 2.9E+07 | 1.0E+07 | 35% | 64% | 3 |
| K228N | K63N | | | | | |
| D104N/K106S/ | D[104]N/K[106]S/ | 2.6E+07 | 7.6E+06 | 29% | 59% | 3 |
| K228N | K63N | | | | | |
| Y155F/K228N | Y[155]F/K63N | 4.5E+07 | 2.4E+06 | 5% | 101% | 2 |
| D104N/K106S/ | D[104]N/K[106]S/ | 5.9E+07 | 1.1E+07 | 19% | 132% | 2 |
| Y155F/K228N | Y[155]F/K63N | | | | | |
| I251S | I86S | 5.9E+07 | 1.2E+07 | 21% | 132% | 13 |
| D85N/I251S | D[85]N/I86S | 5.6E+07 | 1.1E+07 | 20% | 124% | 5 |
| D85N/D104N/ | D[85]N/D[104]N/ | 3.3E+07 | 6.4E+06 | 19% | 75% | 5 |
| K106S/I251S | K[106]S/I86S | | | | | |
| A103N/N105S/ | A[103]N/N[105]S/ | 3.9E+07 | 2.6E+07 | 67% | 87% | 3 |
| I251S | I86S | | | | | |
| D104N/K106S/ | D[104]N/K[106]S/ | 2.9E+07 | 1.1E+06 | 4% | 66% | 2 |
| I251S | I86S | | | | | |
| Y155F/I251S | Y[155]F/I86S | 6.7E+07 | 5.9E+06 | 9% | 149% | 2 |
| A262S | A95bS | 2.4E+07 | 1.0E+07 | 42% | 54% | 8 |
| K413N | K243N | 2.9E+07 | 1.7E+07 | 58% | 64% | 5 |
| E410N | E240N | 1.3E+08 | 8.6E+07 | 65% | 297% | 21 |
| E410N* | E240N* | 3.0E+07 | 1.1E+07 | 36% | 66% | 11 |
| E239N | E74N | 2.0E+07 | 1.1E+07 | 58% | 44% | 9 |
| T241N/H243S | T76N/H78S | 1.9E+07 | 5.7E+05 | 3% | 42% | 2 |
| K247N/N249S | K82N/N84S | 5.4E+07 | 1.7E+07 | 32% | 122% | 11 |
| Y155F/K247N/ | Y[155]F/K82N/ | 5.1E+07 | 9.6E+06 | 19% | 113% | 4 |
| N249S | N84S | | | | | |
| A103N/N105S/ | A[103]N/N[105]S/ | 4.0E+07 | 5.2E+06 | 13% | 90% | 6 |
| K247N/N249S | K82N/N84S | | | | | |
| D104N/K106S/ | D[104]N/K[106]S/ | 3.2E+07 | 3.3E+06 | 10% | 72% | 2 |
| K247N/N249S | K82N/N84S | | | | | |
| D104N/K106S/ | D[104]N/K[106]S/ | 3.2E+07 | 1.1E+07 | 36% | 71% | 3 |
| Y155F/K247N/N249S | Y[155]F/K82N/N84S | | | | | |
| L321N | L153N | 1.6E+07 | 2.0E+06 | 13% | 35% | 2 |
| F314N/H315S | F145N/H147S | No | n.d. | n.d. | 0% | 4 |
| | | Activity | | | | |
| S319N/L321S | S151N/L153S | 2.8E+07 | 2.2E+07 | 78% | 64% | 3 |
| N260S | N95S | 1.8E+07 | 1.2E+07 | 66% | 39% | 13 |
| D104N/K106S/ | D[104]N/K[106]S/ | 1.3E+07 | 6.6E+06 | 51% | 29% | 2 |
| N260S | N95S | | | | | |
| Y155F/N260S | Y[155]F/N95S | 1.9E+07 | 1.6E+07 | 83% | 43% | 2 |
| D104N/K106S/ | D[104]N/K[106]S/ | 4.3E+06 | 2.0E+06 | 46% | 10% | 2 |
| Y155F/N260S | Y[155]F/N95S | | | | | |
| Y284N | Y117N | 3.5E+07 | 1.5E+07 | 42% | 78% | 8 |
| G317N | G149N | No | n.d. | n.d. | 0% | 5 |
| | | Activitv | | | | |
| R318N/A320S | R150N/A152S | No | n.d. | n.d. | 0% | 8 |
| | | Activity | | | | |
| R318A | R150A | 4.9E+07 | 7.4E+06 | 15% | 108% | 3 |
| R318E | R150E | 1.7E+07 | 4.2E+06 | 25% | 38% | 3 |
| R318Y | R150Y | 7.0E+07 | 7.0E+06 | 10% | 156% | 3 |
| R312Q | R143Q | 1.1E+07 | 1.8E+06 | 17% | 23% | 3 |
| R312A | R143A | 4.6E+06 | 9.3E+05 | 20% | 10% | 2 |
| R312Y | R143Y | 1.2E+07 | 4.2E+06 | 36% | 27% | 2 |
| R312L | R143L | 2.4E+07 | 9.4E+06 | 39% | 54% | 2 |
| V202M | V38M | 6.6E+07 | 2.6E+07 | 39% | 148% | 2 |
| V202Y | V38Y | 2.5E+07 | 1.6E+06 | 6% | 56% | 2 |
| D203M | D39M | 4.5E+07 | 1.9E+07 | 42% | 101% | 5 |
| D203Y | D39Y | 3.0E+07 | 2.8E+06 | 9% | 67% | 4 |
| A204M | A40M | 1.8E+07 | 1.2E+07 | 67% | 40% | 5 |
| A204Y | A40Y | 4.6E+07 | 7.6E+06 | 16% | 103% | 2 |
| K400A/R403A | K230A/R233A | 5.3E+06 | 6.9E+05 | 13% | 12% | 2 |
| K400E/R403E | K230E/R233E | No | n.d. | n.d. | 0% | 4 |
| | | Activity | | | | |
| R403A | R233A | 1.4E+07 | 3.0E+06 | 22% | 31% | 7 |
| R403E | R233E | 5.5E+06 | 1.5E+06 | 28% | 12% | 6 |
| K400A | K230A | 2.0E+07 | 3.1E+06 | 16% | 44% | 2 |
| K400E | K230E | 9.5E+06 | 1.1E+06 | 12% | 21% | 2 |
| K293E | K126E | 8.1E+06 | 5.4E+05 | 7% | 18% | 2 |
| K293A | K126A | 2.1E+07 | 4.4E+06 | 21% | 46% | 2 |
| R333A | R165A | No | n.d. | n.d. | 0% | 2 |
| | | Activity | | | | |
| R333E | R165E | No | n.d. | n.d. | 0% | 2 |
| | | Activity | | | | |
| R338A | R170A | 1.6E+08 | 2.5E+07 | 15% | 361% | 2 |
| R338E | R170E | 1.8E+08 | 8.3E+07 | 45% | 408% | 10 |
| R338A/R403A | R170A/R233A | 5.3E+07 | 1.3E+07 | 24% | 119% | 6 |
| R338E/R403E | R170E/R233E | 6.2E+07 | 8.8E+06 | 14% | 138% | 2 |
| K293A/R403A | K126A/R233A | 5.7E+06 | 1.4E+06 | 25% | 13% | 2 |
| K293E/R403E | K126E/R233E | 1.3E+06 | 8.5E+04 | 6% | 3% | 2 |
| K293A/R338A/ | K126A/R170A/ | 2.5E+07 | 9.5E+06 | 39% | 55% | 2 |
| R403A | R233A | | | | | |
| K293E/R338E/ | K126E/R170E/ | 1.7E+07 | 5.7E+05 | 3% | 37% | 2 |
| R403E | R233E | | | | | |
| R318A/R403A | R150A/R233A | 1.5E+07 | 1.3E+06 | 9% | 33% | 2 |
| R318E/R403E | R150E/R233E | 1.2E+06 | 3.8E+05 | 33% | 3% | 2 |
| R318Y/E410N | R150Y/E240N | 7.5E+07 | 2.7E+07 | 35% | 168% | 21 |
| R338E/E410N | R170E/E240N | 4.6E+08 | 1.7E+08 | 38% | 1018% | 8 |
| R338E/R403E/ | R170E/R233E/ | 7.8E+07 | 3.7E+07 | 47% | 175% | 7 |
| E410N | E240N | | | | | |
| R318Y/R338E/ | R150Y/R170E/ | 6.5E+07 | 4.6E+06 | 7% | 145% | 2 |
| R403E | R233E | | | | | |
| D203N/F205T/ | D39N/F41T/K63N | 1.4E+07 | 2.5E+06 | 18% | 31% | 2 |
| K228N | | | | | | |
| D203N/F205T/ | D39N/F41T/E240N | 4.2E+07 | 1.7E+07 | 40% | 94% | 6 |
| E410N | | | | | | |
| D203N/F205T/ | D39N/F41T/R170E | 1.0E+08 | 2.3E+07 | 22% | 234% | 2 |
| R338E | | | | | | |
| D203N/F205T/ | D39N/F41T/R170A | 6.2E+07 | 1.4E+07 | 22% | 139% | 3 |
| R338A | | | | | | |
| D203N/F205T/ | D39N/F41T/R150Y | 2.0E+07 | 2.5E+06 | 12% | 45% | 4 |
| R318Y | | | | | | |
| D203N/F205T/ | D39N/F41T/R170E/ | 1.9E+07 | 4.8E+06 | 25% | 42% | 2 |
| R338E/R403E | R233E | | | | | |
| K228N/E410N | K63N/E240N | 8.5E+07 | 3.4E+07 | 40% | 190% | 10 |
| K228N/R338E | K63N/R170E | 2.1E+08 | 6.1E+07 | 29% | 469% | 2 |
| K228N/R338A | K63N/R170A | 2.1E+08 | 4.6E+07 | 22% | 473% | 2 |
| K228N/R318Y | K63N/R150Y | 4.7E+07 | 6.5E+06 | 14% | 105% | 5 |
| K228N/R338E/ | K63N/R170E/ | 4.8E+07 | 8.6E+06 | 18% | 108% | 2 |
| R403E | R233E | | | | | |
| R403E/E410N | R233E/E240N | 2.1E+07 | 1.7E+06 | 8% | 47% | 2 |
| R318Y/R338E/ | R150Y/R170E/E240N | 3.4E+08 | 1.4E+08 | 39% | 770% | 26 |
| E410N | | | | | | |
| D104N/K106S/ | D[104]N/K[106]S/ | 2.6E+08 | 5.9E+07 | 23% | 581% | 4 |
| R318Y/R338E/ | R150Y/R170E/E240N | | | | | |
| E410N | | | | | | |
| Y155F/R318Y/ | Y[155]F/R150Y/ | 3.7E+08 | 1.3E+08 | 33% | 835% | 5 |
| R338E/E410N | R170E/E240N | | | | | |
| K228N/R318Y/ | K63N/R150Y/ | 1.2E+08 | 2.6E+07 | 22% | 272% | 4 |
| E410N | E240N | | | | | |
| R318Y/R403E/ | R150Y/R233E/ | 2.7E+07 | 3.8E+06 | 14% | 59% | 3 |
| E410N | E240N | | | | | |
| R318Y/R338E/ | R150Y/R170E/ | 1.2E+08 | 8.1E+07 | 69% | 262% | 14 |
| R403E/E410N | R233E/E240N | | | | | |
| A103N/N105S/R318Y/ | A[103]N/N[105]S/ | 1.5E+08 | 7.3E+07 | 50% | 327% | 5 |
| R338E/R403E/E410N | R150Y/R170E/R233E/E240N | | | | | |
| D104N/K106S/R318Y/ | D[104]N/K[106]S/ | 1.7E+08 | 7.9E+07 | 47% | 377% | 3 |
| R338E/R403E/E410N | R150Y/R170E/R233E/E240N | | | | | |
| Y155F/R318Y/ | Y[155]F/R150Y/R170E/ | 1.9E+08 | 5.0E+07 | 27% | 418% | 4 |
| R338E/R403E/E410N | R233E/E240N | | | | | |
| A103N/N105S/Y155F/ | A[103]N/N[105]S/ | 1.3E+08 | 1.8E+06 | 1% | 283% | 2 |
| R318Y/R338E/R403E/ | Y[155]/R150Y/R170E/ | | | | | |
| E410N | R233E/E240N | | | | | |
| D104N/K106S/Y155F/ | D[104]N/K[106]S/ | 1.8E+08 | 9.1E+06 | 5% | 394% | 2 |
| R318Y/R338E/R403E/ | Y[155]F/R150Y/R170E/ | | | | | |
| E410N | R233E/E240N | | | | | |
| D203N/F205T/R318Y/ | D39N/F41T/R150Y/ | 3.9E+07 | 2.0E+07 | 52% | 88% | 6 |
| E410N | E240N | | | | | |
| R333S | R165S | 1.1E+05 | 5.5E+04 | 51% | 0.2% | 3 |
| R338L | R170L | 2.0E+08 | 2.3E+07 | 11% | 444% | 3 |
| K316N | K148N | 6.2E+06 | 4.2E+06 | 69% | 14% | 3 |
| K316A | K148A | 6.1E+06 | 8.2E+05 | 13% | 14% | 3 |
| K316E | K148E | 7.1E+05 | 1.4E+05 | 19% | 2% | 3 |
| K316S | K148S | 3.9E+06 | 6.2E+05 | 16% | 9% | 3 |
| K316M | K148M | 3.1E+07 | 1.4E+07 | 46% | 70% | 3 |
| E239S | E74S | 3.4E+07 | 1.8E+07 | 52% | 75% | 3 |
| E239A | E74A | 4.9E+07 | 6.2E+06 | 13% | 110% | 3 |
| E239R | E74R | 5.6E+07 | 1.1E+07 | 19% | 126% | 3 |
| E239K | E74K | 5.1E+07 | 5.1E+06 | 10% | 114% | 3 |
| H257F | H92F | 4.8E+07 | 6.6E+06 | 14% | 108% | 3 |
| H257Y | H92Y | 3.4E+07 | 9.1E+06 | 27% | 75% | 3 |
| H257E | H92E | 2.7E+07 | 1.5E+07 | 57% | 60% | 3 |
| H257S | H92S | 3.5E+07 | 1.3E+07 | 36% | 78% | 3 |
| T412A | T242A | 4.6E+07 | 2.8E+07 | 62% | 103% | 5 |
| T412V | T242V | 5.8E+07 | 3.2E+07 | 55% | 130% | 8 |
| E410N/T412A | E240N/T242A | 8.0E+07 | 1.7E+07 | 21% | 178% | 4 |
| E410N/T412V | E240N/T242V | 8.8E+07 | 2.7E+07 | 30% | 197% | 4 |
| E410Q | E240Q | 1.2E+08 | 7.6E+07 | 63% | 269% | 4 |
| E410S | E240S | 1.1E+08 | 6.6E+07 | 60% | 246% | 12 |
| E410A | E240A | 1.1E+08 | 5.6E+07 | 50% | 248% | 10 |
| E410D | E240D | 6.0E+07 | 1.6E+07 | 27% | 134% | 4 |
| N346D | N178D | 1.9E+07 | 8.5E+06 | 44% | 43% | 4 |
| Y155F/N346D | Y[155]F/N178D | 1.3E+07 | 6.8E+06 | 53% | 29% | 2 |
| N346Y | N178Y | 9.8E+07 | 2.3E+07 | 24% | 218% | 8 |
| Y345A | Y177A | 1.5E+07 | 6.3E+06 | 43% | 32% | 4 |
| Y345T | Y177T | 5.0E+07 | 2.5E+07 | 50% | 112% | 4 |
| T343R | T175R | 1.7E+08 | 1.1E+08 | 66% | 372% | 9 |
| T343E | T175E | 4.0E+07 | 2.3E+07 | 58% | 88% | 4 |
| T343Q | T175Q | 7.1E+07 | 2.2E+07 | 30% | 159% | 3 |
| F342I | F174I | 5.4E+07 | 2.9E+07 | 54% | 121% | 3 |
| T343R/Y345T | T175R/Y177T | 9.3E+07 | 1.8E+07 | 19% | 208% | 3 |
| R318Y/R338E | R150Y/R170E | 1.5E+08 | 5.3E+07 | 36% | 331% | 4 |
| Y259F/K265T/Y345T | Y94F/K98T/Y177T | 5.6E+07 | 1.2E+07 | 21% | 126% | 2 |
| K228N/I251S | K63N/I86S | 2.2E+07 | 5.7E+05 | 3% | 50% | 2 |
| K228N/R318Y/R338E/ | K63N/R150Y/R170E/ | 1.6E+08 | 6.1E+07 | 39% | 349% | 3 |
| R403E/E410N | R233E/E240N | | | | | |
| Y155F/K228N/R318Y/ | Y[155]F/K63N/R150Y/ | 2.0E+08 | 9.3E+06 | 5% | 453% | 2 |
| R338E/R403E/E410N | R170E/R233E/E240N | | | | | |
| D85N/K228N/R318Y/ | D[85]N/K63N/R150Y/ | 1.6E+08 | 2.3E+07 | 15% | 346% | 2 |
| R338E/R403E/E410N | R170E/R233E/E240N | | | | | |
| I251S/R318Y/R338E/ | I86S/R150Y/R170E/ | 1.5E+08 | 4.2E+07 | 27% | 344% | 4 |
| R403E/E410N | R233E/E240N | | | | | |
| D104N/K106S/I251S/ | D[104]N/K[106]S/ | 1.2E+08 | 2.0E+07 | 16% | 271% | 8 |
| R318Y/R338E/R403E/ | I86S/R150Y/R170E/ | | | | | |
| E410N | R233E/E240N | | | | | |
| Y155F/I251S/R318Y/ | Y[155]F/I86S/R150Y/ | 1.7E+08 | 9.2E+06 | 6% | 374% | 2 |
| R338E/R403E/E410N | R170E/R233E/E240N | | | | | |
| I251S/R318Y/R338E/ | I86S/R150Y/R170E/E240N | 3.8E+08 | 6.1E+07 | 16% | 851% | 7 |
| E410N | | | | | | |
| D104N/K106S/I251S/ | D[104]N/K[106]S/ | 1.3E+08 | 3.2E+07 | 24% | 300% | 3 |
| R318Y/R338E/E410N | I86S/R150Y/R170E/E240N | | | | | |
| F314N/K316S | F145N/K148S | 8.8E+04 | 8.2E+04 | 94% | 0.2% | 2 |
| K247N/N249S/R318Y/ | K82N/N84S/R150Y/R170E/ | 1.5E+08 | 4.7E+07 | 30% | 341% | 6 |
| R338E/R403E/E410N | R233E/E240N | | | | | |
| Y155F/K247N/N249S/ | Y[155]F/K82N/N84S/ | 1.8E+08 | 6.1E+07 | 33% | 408% | 6 |
| R318Y/R338E/R403E/ | R150Y/R170E/R233E/ | | | | | |
| E410N | E240N | | | | | |
| A103N/N105S/K247N/ | A[103]N/N[105]S/ | 1.0E+08 | 7.6E+06 | 7% | 232% | 2 |
| N249S/R318Y/R338E/ | K82N/N84S/R150Y/R170E/ | | | | | |
| R403E/E410N | R233E/E240N | | | | | |
| D104N/K106S/K247N/ | D[104]N/K[106]S/ | 8.8E+07 | 6.5E+06 | 7% | 197% | 2 |
| N249S/R318Y/R338E/ | K82N/N84S/R150Y/R170E/ | | | | | |
| R403E/E410N | R233E/E240N | | | | | |
| K247N/N249S/R318Y/ | K82N/N84S/R150Y/ | 2.3E+08 | 6.6E+07 | 28% | 516% | 6 |
| R338E/E410N | R170E/E240N | | | | | |
| Y155F/K247N/N249S/ | Y[155]F/K82N/N84S/ | 3.0E+08 | 1.3E+08 | 42% | 674% | 7 |
| R318Y/R338E/E410N | R150Y/R170E/E240N | | | | | |
| R318Y/R338E/R403E/ | R150Y/R170E/R233E/ | 1.8E+08 | 6.2E+07 | 34% | 401% | 4 |
| E410S | E240S | | | | | |
| R318Y/R338E/E410S | R150Y/R170E/E240S | 3.3E+08 | 1.2E+08 | 37% | 730% | 8 |
| K228N/K247N/N249S | K63N/K82N/N84S | 3.8E+07 | 1.2E+07 | 32% | 86% | 2 |
| D104N/K106S/Y155F/ | D[104]N/K[106]S/ | 6.3E+07 | 3.3E+06 | 5% | 142% | 2 |
| K228N/K247N/N249S | Y[155]F/K63N/K82N/N84S | | | | | |
| D104N/K106S/K228N/ | D[104]N/K[106]S/ | 2.3E+07 | 1.1E+07 | 48% | 51% | 5 |
| K247N/N249S | K63N/K82N/N84S | | | | | |
| Y155F/K228N/K247N/ | Y[155]F/K63N/K82N/ | 5.3E+07 | 5.5E+06 | 10% | 118% | 2 |
| N249S | N84S | | | | | |
| K228N/K247N/N249S/ | K63N/K82N/N84S/R150Y/ | 1.2E+08 | 3.8E+07 | 33% | 258% | 3 |
| R318Y/R338E/R403E/ | R170E/R233E/E240N | | | | | |
| E410N | | | | | | |
| R318Y/R338E/R403E/ | R150Y/R170E/R233E/ | 1.9E+08 | 5.0E+07 | 26% | 424% | 4 |
| E410N/T412V | E240N/T242V | | | | | |
| R318Y/R338E/R403E/ | R150Y/R170E/R233E/ | 2.6E+08 | 7.4E+07 | 29% | 577% | 4 |
| E410N/T412A | E240N/T242A | | | | | |
| R318Y/R338E/R403E/ | R150Y/R170E/R233E/ | 8.0E+07 | 3.4E+07 | 42% | 178% | 4 |
| T412A | T242A | | | | | |
| R318Y/R338E/T412A | R150Y/R170E/T242A | 3.0E+08 | 8.3E+07 | 28% | 661% | 6 |
| R318Y/R338E/E410N/ | R150Y/R170E/E240N/ | 2.4E+08 | 1.4E+08 | 60% | 536% | 4 |
| T412V | T242V | | | | | |
| N260S/R318Y/R338E/ | N95S/R150Y/R170E/ | 5.3E+07 | 6.6E+05 | 1% | 117% | 2 |
| R403E/E410N | R233E/E240N | | | | | |
| D104N/K106S/N260S/ | D[104]N/K[106]S/ | 8.8E+07 | 7.9E+06 | 9% | 196% | 2 |
| R318Y/R338E/R403E/ | N95S/R150Y/R170E/R233E/ | | | | | |
| E410N | E240N | | | | | |
| Y155F/N260S/R318Y/ | Y[155]F/N95S/R150Y/ | 7.0E+07 | 2.4E+07 | 35% | 156% | 2 |
| R338E/R403E/E410N | R170E/R233E/E240N | | | | | |
| R318Y/R338E/N346D/ | R150Y/R170E/N178D/ | 3.1E+07 | 9.1E+06 | 30% | 68% | 2 |
| R403E/E410N | R233E/E240N | | | | | |
| Y155F/R318Y/R338E/ | Y[155]F/R150Y/R170E/ | 6.2E+07 | 1.8E+07 | 30% | 139% | 2 |
| N346D/R403E/E410N | N178D/R233E/E240N | | | | | |
| K247N/N249S/N260S | K82N/N84S/N95S | 2.9E+07 | 2.6E+06 | 9% | 64% | 2 |
| Y155F/K247N/N249S/ | Y[155]F/K82N/N84S/ | 1.9E+07 | 4.2E+06 | 22% | 43% | 2 |
| N260S | N95S | | | | | |
| D104N/K106S/K247N/ | D[104]N/K[106]S/ | 9.8E+06 | 3.0E+06 | 30% | 22% | 2 |
| N249S/N260S | K82N/N84S/N95S | | | | | |
| DI04N/K106S/Y155F/ | D[104]N/K[106]S/ | 8.2E+06 | 3.9E+06 | 47% | 18% | 2 |
| K247N/N249S/N260S | Y[155]F/K82N/N84S/N95S | | | | | |
| K247N/N249S/N260S/ | K82N/N84S/N95S/ | 9.7E+07 | 8.7E+06 | 9% | 217% | 2 |
| R318Y/R338E/R403E/ | R150Y/R170E/R233E/ | | | | | |
| E410N | E240N | | | | | |
| Y155F/N260S/N346D | Y[155]F/N95S/ | 2.2E+06 | 7.4E+05 | 34% | 5% | 2 |
| | N178D | | | | | |
| R318Y/R338E/T343R/ | R150Y/R170E/T175R/ | 5.4E+08 | 1.6E+08 | 29% | 1217% | 3 |
| R403E/E410N | R233E/E240N | | | | | |
| R338E/T343R | R170E/T175R | 6.0E+08 | 1.7E+08 | 29% | 1329% | 4 |

†produced in BHK-21 cells;
*80% glycosylated form of E410N

TABLE 24
Catalytic activity of FIXa variants (k_{cat}/K_{M})

| | Mutation | | | | % of | |
|---|---|---|---|---|---|---|
| Mutation (Mature | (Chymotrypsin | k_{cat}/K_{M} | ±S.D. | | WT | |
| FIX Numbering) | Numbering) | (M⁻¹s⁻¹) | (M⁻¹V⁻¹) | % CV | k_{cat}/K_{M} | n |
| BeneFIX ® | BeneFIX ® | 4.3E+07 | 2.3E+07 | 54% | 92% | 140 |
| Coagulation FIX | Coagulation FIX | | | | | |
| (T148A) | (T[148]A) | | | | | |
| Plasma Purified | Plasma Purified | 5.6E+07 | 2.6E+07 | 46% | 122% | 200 |
| FIXa | FIXa | | | | | |
| Catalyst | Catalyst | 4.6E+07 | 2.5E+07 | 54% | 100% | 33 |
| Biosciences WT | Biosciences WT | | | | | |
| N157D | N[1571D | 2.9E+07 | 8.1E+06 | 28% | 62% | 2 |
| Y155F | Y[155]F | 4.1E+07 | 1.3E+05 | 0% | 90% | 2 |
| A103N/N105S/ | A[103]N/N[105]S/ | 3.9E+07 | 1.4E+06 | 4% | 85% | 2 |
| Y155F | Y[155]F | | | | | |
| D104N/K106S/ | D[104]N/K[106]S/ | 3.6E+07 | 1.0E+06 | 3% | 78% | 2 |
| Y155F | Y[155]F | | | | | |
| A103N/N105S | A[103]N/N[105]S | 3.7E+07 | 1.4E+07 | 38% | 80% | 9 |
| D104N/K106S | D[104]N/K[106]S | 3.8E+07 | 1.3E+07 | 34% | 83% | 9 |
| K106N/V108S | K[106]N/V[108]S | 2.8E+07 | 6.7E+06 | 24% | 60% | 7 |
| D85N | D[85]N | 7.0E+07 | 2.7E+07 | 39% | 153% | 17 |
| T148A | T[1481A | 4.0E+07 | 2.2E+07 | 54% | 88% | 44 |
| T148A† | T[148]A† | 2.3E+07 | 7.6E+06 | 33% | 50% | 7 |
| K5A | K[5]A | 5.5E+07 | 9.3E+06 | 17% | 120% | 4 |
| D64N | D[64]N | 1.0E+07 | 1.9E+06 | 19% | 22% | 2 |
| D64A | D[64]A | 2.5E+06 | 1.1E+06 | 47% | 5% | 2 |
| N167D | N[167]D | 3.1E+07 | 1.1E+07 | 34% | 67% | 2 |
| N167Q | N[167]Q | 3.5E+07 | 1.9E+07 | 53% | 76% | 4 |
| S61A | S[61]A | 4.8E+07 | 2.5E+07 | 52% | 105% | 4 |
| S53A | S[53]A | 3.5E+07 | 1.7E+07 | 48% | 76% | 3 |
| T159A | T[159]A | 3.7E+07 | 1.2E+07 | 33% | 80% | 3 |
| T169A | T[169]A | 4.7E+07 | 2.0E+07 | 43% | 103% | 3 |
| T172A | T[172]A | 5.0E+07 | 2.6E+07 | 52% | 109% | 3 |
| T179A | T[179]A | 5.5E+07 | 1.3E+07 | 23% | 119% | 3 |
| Y155H | Y[155]H | 5.0E+07 | 1.4E+07 | 27% | 109% | 3 |
| Y155Q | Y[155]Q | 5.4E+07 | 2.0E+07 | 36% | 117% | 3 |
| S158A | S[158]A | 3.6E+07 | 1.1E+06 | 3% | 79% | 2 |
| S158D | S[158]D | 4.0E+07 | 9.3E+05 | 2% | 86% | 2 |
| S158E | S[158]E | 3.7E+07 | 3.5E+06 | 9% | 80% | 2 |
| N157Q | N[157]Q | 3.2E+07 | 2.8E+06 | 9% | 70% | 2 |
| D203N/F205T | D39N/F41T | 2.2E+07 | 1.2E+07 | 53% | 49% | 12 |
| D85N/D203N/F205T | D[85]N/D39N/F41T | 3.0E+07 | 6.4E+06 | 22% | 64% | 5 |
| K228N | K63N | 3.6E+07 | 1.7E+07 | 49% | 77% | 13 |
| D85N/K228N | D[85]N/K63N | 4.6E+07 | 1.5E+07 | 32% | 101% | 6 |
| A103N/N105S/K228N | A[103]N/N[105]S/ | 2.9E+07 | 1.0E+07 | 35% | 63% | 3 |
| | K63N | | | | | |
| D104N/K106S/K228N | D[104]N/K[106]S/ | 2.6E+07 | 7.6E+06 | 29% | 57% | 3 |
| | K63N | | | | | |
| Y155F/K228N | Y[155]F/K63N | 4.5E+07 | 2.4E+06 | 5% | 98% | 2 |
| D104N/K106S/Y155F/ | D[104]N/K[106]S/ | 5.9E+07 | 1.1E+07 | 19% | 129% | 2 |
| K228N | Y[155]F/K63N | | | | | |
| I251S | I86S | 5.9E+07 | 1.2E+07 | 21% | 128% | 13 |
| D85N/I251S | D[85]N/I86S | 5.6E+07 | 1.1E+07 | 20% | 121% | 5 |
| D85N/D104N/K106S/ | D[85]N/D[104]N/ | 3.3E+07 | 6.4E+06 | 19% | 73% | 5 |
| I251S | K[106]S/I86S | | | | | |
| A103N/N105S/I251S | A[103]N/N[105]S/ | 3.9E+07 | 2.6E+07 | 67% | 84% | 3 |
| | I86S | | | | | |
| D104N/K106S/I251S | D[104]N/K[106]S/ | 2.9E+07 | 1.1E+06 | 4% | 64% | 2 |
| | I86S | | | | | |
| Y155F/I251S | Y[155]F/I86S | 6.7E+07 | 5.9E+06 | 9% | 145% | 2 |
| A262S | A95bS | 2.4E+07 | 1.0E+07 | 42% | 52% | 8 |
| K413N | K243N | 2.8E+07 | 1.4E+07 | 51% | 60% | 7 |
| E410N | E240N | 1.3E+08 | 7.7E+07 | 60% | 277% | 27 |
| E410N* | E240N* | 3.0E+07 | 1.1E+07 | 36% | 65% | 10 |
| E239N | E74N | 2.0E+07 | 1.1E+07 | 58% | 43% | 9 |
| T241N/H243S | T76N/H78S | 1.9E+07 | 5.7E+05 | 3% | 41% | 2 |
| K247N/N249S | K82N/N84S | 5.4E+07 | 1.7E+07 | 32% | 118% | 11 |
| Y155F/K247N/N249S | Y[155]F/K82N/N84S | 5.1E+07 | 9.6E+06 | 19% | 110% | 4 |
| A103N/N105S/K247N/ | A[103]N/N[105]S/ | 4.0E+07 | 5.2E+06 | 13% | 87% | 6 |
| N249S | K82N/N84S | | | | | |
| D104N/K106S/K247N/ | D[104]N/K[106]S/ | 3.2E+07 | 3.3E+06 | 10% | 69% | 2 |
| N249S | K82N/N84S | | | | | |
| D104N/K106S/Y155F/ | D[104]N/K[106]S/ | 3.2E+07 | 1.1E+07 | 36% | 69% | 3 |
| K247N/N249S | Y[155]F/K82N/N84S | | | | | |
| L321N | L153N | 1.6E+07 | 2.0E+06 | 13% | 34% | 2 |
| F314N/H315S | F145N/H147S | 4.4E+05 | 3.7E+04 | 8% | 1% | 2 |
| K392N/K394S | K222N/K224S | 0.0E+00 | n.d. | n.d. | 0% | 0 |
| S319N/L321S | S151N/L153S | 2.8E+07 | 2.2E+07 | 78% | 62% | 3 |
| N260S | N95S | 1.8E+07 | 1.2E+07 | 66% | 38% | 13 |
| D104N/K106S/N260S | D[104]N/K[106]S/ | 1.3E+07 | 6.6E+06 | 51% | 28% | 2 |
| | N95S | | | | | |
| Y155F/N260S | Y[1551F/N95S | 1.9E+07 | 1.6E+07 | 83% | 42% | 2 |
| D104N/K106S/Y155F/ | D[104]N/K[106]S/ | 4.3E+06 | 2.0E+06 | 46% | 9% | 2 |
| N260S | Y[155]F/N95S | | | | | |
| Y284N | Y117N | 3.5E+07 | 1.5E+07 | 42% | 76% | 8 |
| G317N | G149N | 4.6E+04 | n.d. | n.d. | 0% | 1 |
| R318N/A320S | R150N/A152S | 2.3E+05 | 2.1E+05 | 89% | 1% | 3 |
| R318A | R150A | 4.5E+07 | 6.4E+06 | 14% | 98% | 2 |
| R318E | R150E | 1.7E+07 | 4.2E+06 | 25% | 37% | 3 |
| R318Y | R150Y | 7.0E+07 | 7.0E+06 | 10% | 151% | 3 |
| R312Q | R143Q | 1.1E+07 | 1.8E+06 | 17% | 23% | 3 |
| R312A | R143A | 4.6E+06 | 9.3E+05 | 20% | 10% | 2 |
| R312Y | R143Y | 1.2E+07 | 4.2E+06 | 36% | 26% | 2 |
| R312L | R143L | 2.4E+07 | 9.4E+06 | 39% | 53% | 2 |
| V202M | V38M | 6.6E+07 | 2.6E+07 | 39% | 143% | 2 |
| V202Y | V38Y | 2.5E+07 | 1.6E+06 | 6% | 55% | 2 |
| D203M | D39M | 4.5E+07 | 1.9E+07 | 42% | 98% | 5 |
| D203Y | D39Y | 3.0E+07 | 2.8E+06 | 9% | 65% | 4 |
| A204M | A40M | 1.8E+07 | 1.2E+07 | 67% | 39% | 5 |
| A204Y | A40Y | 4.6E+07 | 7.6E+06 | 16% | 100% | 2 |
| K400A/R403A | K230A/R233A | 5.3E+06 | 6.9E+05 | 13% | 12% | 2 |
| K400E/R403E | K230E/R233E | 4.3E+05 | 3.1E+04 | 7% | 1% | 3 |
| R403A | R233A | 1.4E+07 | 3.0E+06 | 22% | 30% | 7 |
| R403E | R233E | 5.5E+06 | 1.5E+06 | 28% | 12% | 6 |
| K400A | K230A | 2.0E+07 | 3.1E+06 | 16% | 43% | 2 |
| K400E | K230E | 9.5E+06 | 1.1E+06 | 12% | 21% | 2 |
| K293E | K126E | 8.1E+06 | 5.4E+05 | 7% | 17% | 2 |
| K293A | K126A | 2.1E+07 | 4.4E+06 | 21% | 45% | 2 |
| R333A | R165A | 1.6E+05 | 1.1E+04 | 7% | 0% | 2 |
| R333E | R165E | 1.3E+04 | n.d. | n.d. | 0% | 1 |
| R338A | R170A | 1.6E+08 | 2.5E+07 | 15% | 350% | 2 |
| R338E | R170E | 1.8E+08 | 8.3E+07 | 45% | 396% | 10 |
| R338A/R403A | R170A/R233A | 5.3E+07 | 1.3E+07 | 24% | 115% | 6 |
| R338E/R403E | R170E/R233E | 6.2E+07 | 8.8E+06 | 14% | 134% | 2 |
| K293A/R403A | K126A/R233A | 5.7E+06 | 1.4E+06 | 25% | 12% | 2 |
| K293E/R403E | K126E/R233E | 1.3E+06 | 8.5E+04 | 6% | 3% | 2 |
| K293A/R338A/R403A | K126A/R170A/R233A | 2.5E+07 | 9.5E+06 | 39% | 53% | 2 |
| K293E/R338E/R403E | K126E/R170E/R233E | 1.7E+07 | 5.7E+05 | 3% | 36% | 2 |
| R318A/R403A | R150A/R233A | 1.5E+07 | 1.3E+06 | 9% | 32% | 2 |
| R318E/R403E | R150E/R233E | 1.2E+06 | 3.8E+05 | 33% | 3% | 2 |
| R318Y/E410N | R150Y/E240N | 7.5E+07 | 2.7E+07 | 35% | 163% | 21 |
| R338E/E410N | R170E/E240N | 4.4E+08 | 1.5E+08 | 33% | 950% | 12 |
| R338E/R403E/E410N | R170E/R233E/E240N | 1.9E+08 | 1.4E+08 | 72% | 411% | 17 |
| Y155F/R338E/R403E/ | Y[155]F/R170E/ | 1.8E+08 | 6.0E+07 | 32% | 401% | 2 |
| E410N | R233E/E240N | | | | | |
| R318Y/R338E/R403E | R150Y/R170E/R233E | 6.2E+07 | 6.3E+06 | 10% | 134% | 3 |
| Y155F/R318Y/R338E/ | Y[155]F/R150Y/ | 8.7E+07 | 5.1E+07 | 58% | 189% | 2 |
| R403E | R170E/R233E | | | | | |
| D203N/F205T/K228N | D39N/F41T/K63N | 1.4E+07 | 2.5E+06 | 18% | 30% | 2 |
| D203N/F205T/E410N | D39N/F41T/E240N | 4.2E+07 | 1.7E+07 | 40% | 91% | 6 |
| D203N/F205T/R338E | D39N/F41T/R170E | 1.0E+08 | 2.3E+07 | 22% | 228% | 2 |
| D203N/F205T/R338A | D39N/F41T/R170A | 6.2E+07 | 1.4E+07 | 22% | 135% | 3 |
| D203N/F205T/R318Y | D39N/F41T/R150Y | 2.0E+07 | 2.5E+06 | 12% | 44% | 4 |
| D203N/F205T/ | D39N/F41T/R170E/ | 1.9E+07 | 4.8E+06 | 25% | 41% | 2 |
| R338E/R403E | R233E | | | | | |
| K228N/E410N | K63N/E240N | 8.5E+07 | 3.4E+07 | 40% | 184% | 10 |
| K228N/R338E | K63N/R170E | 2.1E+08 | 6.1E+07 | 29% | 455% | 2 |
| K228N/R338A | K63N/R170A | 2.1E+08 | 4.6E+07 | 22% | 459% | 2 |
| K228N/R318Y | K63N/R150Y | 4.7E+07 | 6.5E+06 | 14% | 102% | 5 |
| K228N/R338E/R403E | K63N/R170E/R233E | 4.8E+07 | 8.6E+06 | 18% | 105% | 2 |
| R403E/E410N | R233E/E240N | 2.1E+07 | 1.7E+06 | 8% | 46% | 2 |
| R318Y/R338E/E410N | R150Y/R170E/E240N | 3.4E+08 | 1.2E+08 | 37% | 727% | 42 |
| D104N/K106S/R318Y/ | D[104]N/K[106]S/ | 2.6E+08 | 5.9E+07 | 23% | 564% | 4 |
| R338E/E410N | R150Y/R170E/E240N | | | | | |
| Y155F/R318Y/R338E/ | Y[155]F/R150Y/R170E/ | 3.7E+08 | 1.3E+08 | 33% | 810% | 5 |
| E410N | E240N | | | | | |
| K228N/R318Y/E410N | K63N/R150Y/E240N | 1.2E+08 | 2.6E+07 | 22% | 264% | 4 |
| R318Y/R403E/E410N | R150Y/R233E/E240N | 2.5E+07 | 4.7E+06 | 19% | 54% | 5 |
| Y155F/R318Y/R403E/ | Y[155]F/R150Y/R233E/ | 3.6E+07 | 2.9E+07 | 82% | 78% | 2 |
| E410N | E240N | | | | | |
| R318Y/R338E/R403E/ | R150Y/R170E/R233E/ | 1.5E+08 | 8.2E+07 | 56% | 320% | 26 |
| E410N | E240N | | | | | |
| A103N/N105S/R318Y/ | A[103]N/N[105]S/ | 1.5E+08 | 7.3E+07 | 50% | 318% | 5 |
| R338E/R403E/E410N | R150Y/R170E/R233E/E240N | | | | | |
| D104N/K106S/R318Y/ | D[104]N/K[106]S/ | 1.7E+08 | 7.9E+07 | 47% | 366% | 3 |
| R338E/R403E/E410N | R150Y/R170E/R233E/E240N | | | | | |
| Y155F/R318Y/R338E/ | Y[155]F/R150Y/R170E/ | 1.9E+08 | 5.0E+07 | 27% | 406% | 4 |
| R403E/E410N | R233E/E240N | | | | | |
| A103N/N105S/Y155F/ | A[103]N/N[105]S/ | 1.3E+08 | 1.8E+06 | 1% | 274% | 2 |
| R318Y/R338E/R403E/ | Y[155]F/R150Y/R170E/ | | | | | |
| E410N | R233E/E240N | | | | | |
| D104N/K106S/Y155F/ | D[104]N/K[106]S/ | 1.8E+08 | 9.1E+06 | 5% | 382% | 2 |
| R318Y/R338E/R403E/ | Y[155]F/R150Y/R170E/ | | | | | |
| E410N | R233E/E240N | | | | | |
| D203N/F205T/R318Y/ | D39N/F41T/R150Y/ | 3.9E+07 | 2.0E+07 | 52% | 85% | 6 |
| E410N | E240N | | | | | |
| R333S | R165S | 1.1E+05 | 5.5E+04 | 51% | 0% | 3 |
| R338L | R170L | 2.0E+08 | 2.3E+07 | 11% | 431% | 3 |
| K316N | K148N | 6.2E+06 | 4.2E+06 | 69% | 13% | 3 |
| K316A | K148A | 6.1E+06 | 8.2E+05 | 13% | 13% | 3 |
| K316E | K148E | 7.1E+05 | 1.4E+05 | 19% | 2% | 3 |
| K316S | K148S | 3.9E+06 | 6.2E+05 | 16% | 9% | 3 |
| K316M | K148M | 3.1E+07 | 1.4E+07 | 46% | 68% | 3 |
| E239S | E74S | 3.4E+07 | 1.8E+07 | 52% | 73% | 3 |
| E239A | E74A | 4.9E+07 | 6.2E+06 | 13% | 107% | 3 |
| E239R | E74R | 5.6E+07 | 1.1E+07 | 19% | 122% | 3 |
| E239K | E74K | 5.1E+07 | 5.1E+06 | 10% | 111% | 3 |
| H257F | H92F | 4.8E+07 | 6.6E+06 | 14% | 105% | 3 |
| H257Y | H92Y | 3.4E+07 | 9.1E+06 | 27% | 73% | 3 |
| H257E | H92E | 2.7E+07 | 1.5E+07 | 57% | 58% | 3 |
| H257S | H92S | 3.5E+07 | 1.3E+07 | 36% | 76% | 3 |
| T412A | T242A | 4.6E+07 | 2.8E+07 | 62% | 100% | 5 |
| T412V | T242V | 5.8E+07 | 3.2E+07 | 55% | 126% | 8 |
| E410N/T412A | E240N/T242A | 8.0E+07 | 1.7E+07 | 21% | 173% | 4 |
| E410N/T412V | E240N/T242V | 8.8E+07 | 2.7E+07 | 30% | 192% | 4 |
| E410Q | E240Q | 1.2E+08 | 7.6E+07 | 63% | 261% | 4 |
| E410S | E240S | 1.1E+08 | 6.6E+07 | 60% | 239% | 12 |
| E410A | E240A | 1.1E+08 | 5.6E+07 | 50% | 241% | 10 |
| E410D | E240D | 6.0E+07 | 1.6E+07 | 27% | 130% | 4 |
| N346D | N178D | 1.9E+07 | 8.5E+06 | 44% | 42% | 4 |
| Y155F/N346D | Y[155]F/N178D | 1.3E+07 | 6.8E+06 | 53% | 28% | 2 |
| N346Y | N178Y | 9.8E+07 | 2.3E+07 | 24% | 212% | 8 |
| Y345A | Y177A | 1.5E+07 | 6.3E+06 | 43% | 32% | 4 |
| Y345T | Y177T | 5.0E+07 | 2.5E+07 | 50% | 108% | 4 |
| T343R | T175R | 1.4E+08 | 1.0E+08 | 70% | 313% | 12 |
| T343E | T175E | 4.0E+07 | 2.3E+07 | 58% | 86% | 4 |
| T343Q | T175Q | 7.1E+07 | 2.2E+07 | 30% | 154% | 3 |
| F342I | F174I | 5.4E+07 | 2.9E+07 | 54% | 118% | 3 |
| T343R/Y345T | T175R/Y177T | 9.3E+07 | 1.8E+07 | 19% | 202% | 3 |
| R318Y/R338E | R150Y/R170E | 1.5E+08 | 5.3E+07 | 36% | 322% | 4 |
| Y259F/K265T/Y345T | Y94F/K98T/Y177T | 5.6E+07 | 1.2E+07 | 21% | 122% | 2 |
| K228N/I251S | K63N/I86S | 2.2E+07 | 5.7E+05 | 3% | 48% | 2 |
| K228N/R318Y/R338E/ | K63N/R150Y/R170E/ | 1.6E+08 | 6.1E+07 | 39% | 339% | 3 |
| R403E/E410N | R233E/E240N | | | | | |
| Y155F/K228N/R318Y/ | Y[155]F/K63N/R150Y/ | 1.6E+08 | 4.1E+07 | 25% | 356% | 5 |
| R338E/R403E/E410N | R170E/R233E/E240N | | | | | |
| D85N/K228N/R318Y/ | D[85]N/K63N/R150Y/ | 1.6E+08 | 2.3E+07 | 15% | 336% | 2 |
| R338E/R403E/E410N | R170E/R233E/E240N | | | | | |
| I251S/R318Y/R338E/ | I86S/R150Y/R170E/ | 1.5E+08 | 4.2E+07 | 27% | 334% | 4 |
| R403E/E410N | R233E/E240N | | | | | |
| D104N/K106S/I251S/ | D[104]N/K[106]S/ | 1.2E+08 | 2.0E+07 | 16% | 263% | 8 |
| R318Y/R338E/R403E/ | I86S/R150Y/R170E/R233E/ | | | | | |
| E410N | E240N | | | | | |
| Y155F/I251S/R318Y/ | D[104]N/K[106]S/ | 1.7E+08 | 9.2E+06 | 6% | 363% | 2 |
| R338E/R403E/E410N | I86S/R150Y/R170E/R233E/ | | | | | |
| | E240N | | | | | |
| I251S/R318Y/R338E/ | I86S/R150Y/R170E/ | 3.9E+08 | 7.4E+07 | 19% | 849% | 10 |
| E410N | E240N | | | | | |
| D104N/K106S/I25IS/ | D[104]N/K[106]S/ | 1.3E+08 | 3.2E+07 | 24% | 291% | 3 |
| R318Y/R338E/E410N | I86S/R150Y/R170E/E240N | | | | | |
| F314N/K316S | F145N/K148S | 8.8E+04 | 8.2E+04 | 94% | 0% | 2 |
| K247N/N249S/R318Y/ | K82N/N84S/R150Y/ | 1.5E+08 | 4.7E+07 | 30% | 331% | 6 |
| R338E/R403E/E410N | R170E/R233E/E240N | | | | | |
| Y155F/K247N/N249S/ | Y[155]F/K82N/N84S/ | 1.9E+08 | 5.7E+07 | 30% | 405% | 10 |
| R318Y/R338E/R403E/ | R150Y/R170E/R233E/E240N | | | | | |
| E410N | | | | | | |
| A103N/N105S/K247N/ | A[103]N/N[105]S/ | 1.5E+08 | 4.2E+07 | 28% | 324% | 6 |
| N249S/R318Y/R338E/ | K82N/N84S/R150Y/R170E/ | | | | | |
| R403E/E410N | R233E/E240N | | | | | |
| D104N/K106S/K247N/ | D[104]N/K[106]S/ | 8.8E+07 | 6.5E+06 | 7% | 192% | 2 |
| N249S/R318Y/R338E/ | K82N/N84S/R150Y/R170E/ | | | | | |
| R403E/E410N | R233E/E240N | | | | | |
| D104N/K106S/Y155F/ | D[104]N/K[106]S/ | 1.3E+08 | 7.3E+07 | 54% | 292% | 6 |
| K247N/N249S/R318Y/ | Y[155]F/K82N/N84S/R150Y/ | | | | | |
| R338E/R403E/E410N | R170E/R233E/E240N | | | | | |
| K247N/N249S/R318Y/ | K82N/N84S/R150Y/ | 2.3E+08 | 6.6E+07 | 28% | 501% | 6 |
| R338E/E410N | R170E/E240N | | | | | |
| Y155F/K247N/N249S/ | Y[155]F/K82N/N84S/ | 3.3E+08 | 1.3E+08 | 39% | 717% | 9 |
| R318Y/R338E/E410N | R150Y/R170E/E240N | | | | | |
| R318Y/R338E/R403E/ | R150Y/R170E/R233E/ | 2.1E+08 | 6.1E+07 | 29% | 458% | 7 |
| E410S | E240S | | | | | |
| R318Y/R338E/E410S | R150Y/R170E/E240S | 3.3E+08 | 1.2E+08 | 37% | 708% | 8 |
| K228N/K247N/N249S | K63N/K82N/N84S | 3.8E+07 | 1.2E+07 | 32% | 83% | 2 |
| D104N/K106S/Y155F/ | D[104]N/K[106]S/ | 6.3E+07 | 3.3E+06 | 5% | 137% | 2 |
| K228N/K247N/N249S | Y[155]F/K63N/K82N/N84S | | | | | |
| D104N/K106S/K228N/ | D[104]N/K[106]S/ | 2.3E+07 | 1.1E+07 | 48% | 49% | 5 |
| K247N/N249S | K63N/K82N/N84S | | | | | |
| Y155F/K228N/K247N/ | Y[155]F/K63N/K82N/ | 5.3E+07 | 5.5E+06 | 10% | 115% | 2 |
| N249S | N84S | | | | | |
| K228N/K247N/N249S/ | K63N/K82N/N84S/R150Y/ | 1.6E+08 | 8.4E+07 | 51% | 352% | 17 |
| R318Y/R338E/R403E/ | R170E/R233E/E240N | | | | | |
| E410N | | | | | | |
| D104N/K106S/K228N/ | D[104]N/K[106]S/ | 1.1E+08 | 4.4E+07 | 40% | 239% | 7 |
| K247N/N249S/R318Y/ | K63N/K82N/N84S/R150Y/R170E/ | | | | | |
| R338E/R403E/E410N | R233E/E240N | | | | | |
| Y155F/K228N/K247N/ | Y[155]F/K63N/K82N/N84S/ | 1.2E+08 | 5.3E+07 | 44% | 263% | 5 |
| N249S/R318Y/R338E/ | R150Y/R170E/R233E/E240N | | | | | |
| R403E/E410N | | | | | | |
| R318Y/R338E/R403E/ | R150Y/R170E/R233E/ | 1.6E+08 | 6.3E+07 | 40% | 342% | 6 |
| E410N/T412V | E240N/T242V | | | | | |
| R318Y/R338E/R403E/ | R150Y/R170E/R233E/ | 2.5E+08 | 9.2E+07 | 37% | 538% | 6 |
| E410N/T412A | E240N/T242A | | | | | |
| R318Y/R338E/R403E/ | R150Y/R170E/R233E/ | 8.0E+07 | 3.4E+07 | 42% | 173% | 4 |
| T412A | T242A | | | | | |
| R318Y/R338E/T412A | R150Y/R170E/T242A | 3.0E+08 | 8.3E+07 | 28% | 642% | 6 |
| R318Y/R338E/E410N/ | R150Y/R170E/E240N/ | 2.6E+08 | 1.2E+08 | 46% | 571% | 11 |
| T412V | T242V | | | | | |
| N260S/R318Y/R338E/ | N95S/R150Y/R170E/ | 5.3E+07 | 6.6E+05 | 1% | 114% | 2 |
| R403E/E410N | R233E/E240N | | | | | |
| D104N/K106S/N260S/ | D[104]N/K[106]S/ | 8.8E+07 | 7.9E+06 | 9% | 190% | 2 |
| R318Y/R338E/R403E/ | N95S/R150Y/R170E/R233E/ | | | | | |
| E410N | E240N | | | | | |
| Y155F/N260S/R318Y/ | Y[155]F/N95S/R150Y/ | 7.0E+07 | 2.4E+07 | 35% | 152% | 2 |
| R338E/R403E/E410N | R170E/R233E/E240N | | | | | |
| R318Y/R338E/N346D/ | R150Y/R170E/N178D/ | 3.1E+07 | 9.1E+06 | 30% | 66% | 2 |
| R403E/E410N | R233E/E240N | | | | | |
| Y155F/R318Y/R338E/ | Y[155]F/R150Y/R170E/ | 6.2E+07 | 1.8E+07 | 30% | 135% | 2 |
| N346D/R403E/E410N | N178D/R233E/E240N | | | | | |
| K247N/N249S/N260S | K82N/N84S/N95S | 2.9E+07 | 2.6E+06 | 9% | 62% | 2 |
| Y155F/K247N/N249S/ | Y[155]F/K82N/N84S/ | 1.9E+07 | 4.2E+06 | 22% | 42% | 2 |
| N260S | N95S | | | | | |
| D104N/K106S/K247N/ | D[104]N/K[106]S/ | 9.8E+06 | 3.0E+06 | 30% | 21% | 2 |
| N249S/N260S | K82N/N84S/N95S | | | | | |
| D104N/K106S/Y155F/ | D[104]N/K[106]S/ | 8.2E+06 | 3.9E+06 | 47% | 18% | 2 |
| K247N/N249S/N260S | Y[155]F/K82N/N84S/N95S | | | | | |
| K247N/N249S/N260S/ | K82N/N84S/N95S/R150Y/ | 6.7E+07 | 2.6E+07 | 38% | 145% | 6 |
| R318Y/R338E/R403E/ | R170E/R233E/E240N | | | | | |
| E410N | | | | | | |
| Y155F/K247N/N249S/ | Y[155]F/K82N/N84S/N95S/ | 5.7E+07 | 3.6E+07 | 64% | 124% | 5 |
| N260S/R318Y/R338E/ | R150Y/R170E/R233E/E240N | | | | | |
| R403E/E410N | | | | | | |
| Y155F/N260S/N346D | Y[155]F/N95S/N178D | 2.2E+06 | 7.4E+05 | 34% | 5% | 2 |
| R318Y/R338E/T343R/ | R150Y/R170E/T175R/ | 4.2E+08 | 1.4E+08 | 33% | 907% | 13 |
| R403E/E410N | R233E/E240N | | | | | |
| Y155F/R318Y/R338E/ | Y[155]F/R150Y/R170E/ | 3.0E+08 | 8.3E+07 | 28% | 640% | 4 |
| T343R/R403E/E410N | T175R/R233E/E240N | | | | | |
| D104N/K106S/R318Y/ | D[104]N/K[106]S/ | 2.2E+08 | 1.2E+08 | 52% | 487% | 5 |
| R338E/T343R/R403E/ | R150Y/R170E/T175R/R233E/ | | | | | |
| E410N | E240N | | | | | |
| R338E/T343R | R170E/T175R | 5.2E+08 | 1.6E+08 | 31% | 1120% | 7 |
| T343R/N346Y | T175R/N178Y | 9.6E+07 | 4.4E+07 | 46% | 208% | 11 |
| R318Y/R338E/N346Y/ | R150Y/R170E/N178Y/ | 1.2E+08 | 2.1E+07 | 16% | 270% | 3 |
| R403E/E410N | R233E/E240N | | | | | |
| R318Y/R338E/T343R/ | R150Y/R170E/T175R/ | 3.1E+08 | 1.1E+08 | 37% | 663% | 5 |
| N346Y/R403E/E410N | N178Y/R233E/E240N | | | | | |
| T343R/N346D | T175R/N178D | 1.6E+07 | 2.6E+06 | 16% | 36% | 2 |
| R318Y/R338E/T343R/ | R150Y/R170E/T175R/ | 8.2E+07 | 3.2E+06 | 4% | 177% | 2 |
| N346D/R403E/E410N | N178D/R233E/E240N | | | | | |
| R318Y/R338E/Y345A/ | R150Y/R170E/Y177A/ | 8.3E+07 | 3.6E+07 | 44% | 180% | 6 |
| R403E/E410N | R233E/E240N | | | | | |
| R318Y/R338E/Y345A/ | R150Y/R170E/Y177A/ | 2.3E+07 | 7.6E+06 | 33% | 49% | 3 |
| N346D/R403E/E410N | N178D/R233E/E240N | | | | | |
| Y155F/K247N/N249S/ | Y[155]F/K82N/N84S/ | 9.5E+07 | 6.6E+07 | 69% | 206% | 5 |
| R318Y/R338E/R403E | R150Y/R170E/R233E | | | | | |
| K247N/N249S/R318Y/ | K82N/N84S/R150Y/ | 2.3E+08 | 1.6E+08 | 71% | 496% | 2 |
| R338E/R403E | R170E/R233E | | | | | |
| Y155F/K247N/N249S/ | Y[155]F/K82N/N84S/ | 1.0E+07 | 4.5E+06 | 45% | 22% | 3 |
| R318Y/R403E/E410N | R150Y/R233E/E240N | | | | | |
| K247N/N249S/R318Y/ | K82N/N84S/R150Y/R233E/ | 2.7E+07 | 1.2E+07 | 44% | 58% | 10 |
| R403E/E410N | E240N | | | | | |
| Y155F/K247N/N249S/ | Y[155]F/K82N/N84S/ | 1.1E+08 | 2.4E+07 | 23% | 229% | 3 |
| R338E/R403E/E410N | R170E/R233E/E240N | | | | | |
| K247N/N249S/R338E/ | K82N/N84S/R170E/ | 1.9E+08 | 2.9E+07 | 15% | 422% | 2 |
| R403E/E410N | R233E/E240N | | | | | |
| R318Y/R338E/T343R/ | R150Y/R170E/T175R/ | 1.6E+08 | 7.4E+07 | 45% | 357% | 4 |
| R403E | R233E | | | | | |
| Y155F/R318Y/R338E/ | Y[155]F/R150Y/R170E/ | 2.6E+08 | 1.7E+08 | 65% | 563% | 4 |
| T343R/R403E | T175R/R233E | | | | | |
| R318Y/R338E/T343R/ | R150Y/R170E/T175R/ | 3.4E+08 | 1.6E+08 | 48% | 728% | 16 |
| E410N | E240N | | | | | |
| Y155F/R318Y/R338E/ | Y[155]F/R150Y/R170E/ | 3.7E+08 | 1.2E+08 | 32% | 794% | 4 |
| T343R/E410N | T175R/E240N | | | | | |
| R318Y/T343R/R403E/ | R150Y/T175R/R233E/ | 5.8E+07 | 1.8E+07 | 31% | 125% | 3 |
| E410N | E240N | | | | | |
| Y155F/R318Y/T343R/ | Y[155]F/R150Y/T175R/ | 2.6E+08 | 5.0E+07 | 19% | 571% | 2 |
| R403E/E410N | R233E/E240N | | | | | |
| R338E/T343R/R403E/ | R170E/T175R/R233E/ | 3.0E+08 | 8.2E+07 | 27% | 650% | 2 |
| E410N | E240N | | | | | |
| Y155F/R338E/T343R/ | Y[155]F/R170E/T175R/ | 2.4E+08 | 1.0E+08 | 42% | 524% | 4 |
| R403E/E410N | R233E/E240N | | | | | |
| Y155F/K247N/N249S/ | Y[155]F/K82N/N84S/ | 4.0E+08 | 1.5E+08 | 37% | 864% | 11 |
| R318Y/R338E/T343R/ | R150Y/R170E/T175R/ | | | | | |
| R403E/E410N | R233E/E240N | | | | | |
| K247N/N249S/R318Y/ | K82N/N84S/R150Y/R170E/ | 3.8E+08 | 1.5E+08 | 40% | 824% | 5 |
| R338E/T343R/R403E/ | T175R/R233E/E240N | | | | | |
| E410N | | | | | | |
| K228N/I251S/R318Y/ | K63N/I86S/R150Y/R170E/ | 2.1E+08 | 7.2E+07 | 34% | 463% | 7 |
| R338E/R403E/E410N | R233E/E240N | | | | | |
| Y155F/K228N/I251S/ | Y[155]F/K63N/ | 1.4E+08 | 5.0E+07 | 37% | 296% | 5 |
| R318Y/R338E/ | I86S/R150Y/R170E/ | | | | | |
| R403E/E410N | R233E/E240N | | | | | |
| N260S/R318Y/R338E/ | N95S/R150Y/R170E/T175R/ | 2.9E+08 | 1.1E+08 | 38% | 638% | 7 |
| T343R/R403E/E410N | R233E/E240N | | | | | |
| Y155F/N260S/R318Y/ | Y[155]F/N95S/R150Y/ | 1.5E+08 | 6.0E+07 | 39% | 335% | 5 |
| R338E/T343R/R403E/ | R170E/T175R/R233E/ | | | | | |
| E410N | E240N | | | | | |
| K228N/K247N/N249S/ | K63N/K82N/N84S/R150Y/ | 4.1E+08 | 1.4E+08 | 34% | 880% | 12 |
| R318Y/R338E/T343R/ | R170E/T175R/R233E/ | | | | | |
| R403E/E410N | E240N | | | | | |
| Y155F/K228N/K247N/ | Y[155]F/K63N/ | 3.0E+08 | 1.1E+08 | 37% | 646% | 5 |
| N249S/R318Y/R338E/ | K82N/N84S/R150Y/R170E/ | | | | | |
| T343R/R403E/E410N | T175R/R233E/E240N | | | | | |
| Y155F/R338E/T343R/ | Y[155]F/R170E/T175R/ | 2.0E+08 | 7.7E+07 | 39% | 429% | 5 |
| R403E | R233E | | | | | |
| R338E/T343R/R403E | R170E/T175R/R233E | 3.1E+08 | 9.6E+07 | 31% | 663% | 2 |
| Y155F/R338E/T343R/ | Y[155]F/R170E/T175R/ | 2.9E+08 | 1.0E+08 | 35% | 629% | 6 |
| R403E/E410S | R233E/E240S | | | | | |
| Y155F/N260S/R338E/ | Y[155]F/N95S/R170E/ | 9.4E+07 | 3.1E+07 | 33% | 203% | 6 |
| T343R/R403E | T175R/R233E | | | | | |
| Y155F/I251S/R338E/ | Y[155]F/I86S/R170E/ | 3.0E+08 | 1.6E+07 | 5% | 651% | 2 |
| T343R/R403E | T175R/R233E | | | | | |
| R318Y/R338E/T343R/ | R150Y/R170E/T175R/ | 4.4E+08 | 1.7E+08 | 39% | 962% | 14 |
| R403E/E410S | R233E/E240S | | | | | |
| Y155F/K247N/N249S/ | Y[155]F/K82N/N84S/ | 8.5E+07 | 2.7E+07 | 31% | 184% | 4 |
| T343R/R403E | T175R/R233E | | | | | |
| Y155F/K247N/N249S/ | Y[155]F/K82N/N84S/ | 2.9E+08 | 5.0E+06 | 2% | 630% | 2 |
| R318Y/R338E/ | R150Y/R170E/T175R/ | | | | | |
| T343R/R403E | R233E | | | | | |
| K247N/N249S/R318Y/ | K82N/N84S/R150Y/ | 4.1E+08 | 2.2E+08 | 55% | 886% | 4 |
| R338E/T343R/R403E | R170E/T175R/R233E | | | | | |
| Y155F/K247N/N249S/ | Y[155]F/K82N/N84S/ | 3.7E+08 | 1.1E+07 | 3% | 805% | 2 |
| R338E/T343R/R403E/ | R170E/T175R/R233E/ | | | | | |
| E410N | E240N | | | | | |
| K247N/N249S/R338E/ | K82N/N84S/R170E/ | 4.3E+08 | 1.2E+07 | 3% | 930% | 2 |
| T343R/R403E/E410N | T175R/R233E/E240N | | | | | |
| Y155F/K247N/N249S/ | Y[155]F/K82N/N84S/ | 2.9E+08 | 4.1E+07 | 14% | 632% | 2 |
| R318Y/R338E | R150Y/R170E | | | | | |
| Y155F/K247N/N249S/ | Y[155]F/K82N/N84S/ | 2.5E+08 | 9.4E+07 | 37% | 549% | 4 |
| R318Y/T343R | R150Y/T175R | | | | | |
| Y155F/K247N/N249S/ | Y[155]F/K82N/N84S/ | 1.6E+07 | 5.4E+06 | 35% | 34% | 3 |
| R318Y/R403E | R150Y/R233E | | | | | |
| Y155F/K247N/N249S/ | Y[155]F/K82N/N84S/ | 7.2E+07 | 2.5E+07 | 35% | 155% | 3 |
| R318Y/E410N | R150Y/E240N | | | | | |
| Y155F/K247N/N249S/ | Y[155]F/K82N/N84S/ | 1.4E+08 | 5.7E+07 | 41% | 299% | 2 |
| R338E/R403E | R170E/R233E | | | | | |
| Y155F/K247N/N249S/ | Y[155]F/K82N/N84S/ | 7.3E+08 | 2.6E+08 | 36% | 1579% | 2 |
| R338E/T343R | R170E/T175R | | | | | |
| Y155F/K247N/N249S/ | Y[155]F/K82N/N84S/ | 5.0E+08 | 2.8E+08 | 57% | 1091% | 4 |
| R318Y/R338E/T343R/ | R150Y/R170E/T175R/ | | | | | |
| E410N | E240N | | | | | |
| K247N/N249S/R318Y/ | K82N/N84S/R150Y/ | 3.2E+08 | 1.6E+08 | 50% | 687% | 6 |
| R338E/T343R/E410N | R170E/T175R/E240N | | | | | |
| Y155F/K247N/N249S/ | Y[155]F/K82N/N84S/ | 1.6E+08 | 6.2E+07 | 38% | 350% | 2 |
| R318Y/T343R/R403E/ | R150Y/T175R/R233E/ | | | | | |
| E410N | E240N | | | | | |
| K247N/N249S/R318Y/ | K82N/N84S/R150Y/ | 1.3E+08 | 3.9E+07 | 30% | 279% | 7 |
| | T175R/R233E/E240N | | | | | |
| T343R/R403E/E410N | | | | | | |
| Y155F/K247N/N249S/ | Y[155]F/K82N/N84S/ | 4.7E+08 | 3.1E+08 | 66% | 1009% | 8 |
| R338E/E410N | R170E/E240N | | | | | |
| Y155F/K247N/N249S/ | Y[155]F/K82N/N84S/ | 1.3E+08 | 5.1E+07 | 40% | 276% | 2 |
| R318Y/T343R/R403E | R150Y/T175R/R233E | | | | | |
| K247N/N249S/R318Y/ | K82N/N84S/R150Y/ | 3.9E+07 | 2.2E+07 | 57% | 84% | 9 |
| T343R/R403E | T175R/R233E | | | | | |
| Y155F/K247N/N249S/ | Y[155]F/K82N/N84S/ | 3.1E+08 | 2.1E+08 | 67% | 668% | 4 |
| R318Y/T343R/E410N | R150Y/T175R/E240N | | | | | |
| K247N/N249S/R318Y/ | K82N/N84S/R150Y/ | 2.0E+08 | 1.6E+08 | 77% | 439% | 4 |
| T343R/E410N | T175R/E240N | | | | | |
| Y155F/K247N/N249S/ | Y[155]F/K82N/N84S/ | 5.9E+08 | 5.8E+07 | 10% | 1269% | 2 |
| R338E/T343R/R403E | R170E/T175R/R233E | | | | | |
| K247N/N249S/R338E/ | K82N/N84S/R170E/ | 5.6E+08 | 8.8E+07 | 16% | 1215% | 2 |
| T343R/R403E | T175R/R233E | | | | | |
| Y155F/K247N/N249S/ | Y[155]F/K82N/N84S/ | 1.8E+08 | 1.1E+07 | 6% | 391% | 2 |
| R338E/T343R/E410N | R170E/T175R/E240N | | | | | |
| K247N/N249S/R338E/ | K82N/N84S/R170E/ | 3.1E+08 | 1.0E+08 | 33% | 676% | 5 |
| T343R/E410N | T175R/E240N | | | | | |
| Y155F/K247N/N249S/ | Y[155]F/K82N/N84S/ | 2.9E+08 | 8.8E+07 | 30% | 635% | 2 |
| T343R/R403E/E410N | T175R/R233E/E240N | | | | | |
| K247N/N249S/T34 | K82N/N84S/T175R/ | 1.3E+08 | 1.7E+07 | 13% | 285% | 2 |
| 3R/R403E/E410N | R233E/E240N | | | | | |
| Y155F/R318Y/R338E/ | Y[155]F/R150Y/ | 3.6E+08 | 1.5E+08 | 41% | 771% | 7 |
| T343R | R170E/T175R | | | | | |
| R318Y/R338E/T343R | R150Y/R170E/T175R | 1.5E+08 | 3.3E+07 | 22% | 324% | 2 |
| Y155F/R318Y/T343R/ | Y[155]F/R150Y/T175R/ | 7.1E+07 | 1.4E+07 | 20% | 154% | 2 |
| R403E | R233E | | | | | |
| Y155F/T343R/R403E/ | Y[155]F/T175R/R233E/ | 1.5E+08 | 2.4E+07 | 17% | 321% | 2 |
| E410N | E240N | | | | | |
| Y155F/K247N/N249S/ | Y[155]F/K82N/N84S/ | 3.6E+08 | 1.6E+08 | 45% | 772% | 7 |
| R318Y/R338E/T343R | R150Y/R170E/T175R | | | | | |
| K247N/N249S/R318Y/ | K82N/N84S/R150Y/ | 3.9E+08 | 1.6E+08 | 43% | 840% | 4 |
| R338E/T343R | R170E/T175R | | | | | |
| Y155F/K247N/N249S/ | Y[155]F/K82N/N84S/ | 2.8E+08 | 1.1E+08 | 38% | 599% | 5 |
| T343R/E410N | T175R/E240N | | | | | |
| Y155F/K247N/N249S/ | Y[155]F/K82N/N84S/ | 2.4E+07 | 1.4E+07 | 59% | 53% | 7 |
| R403E/E410N | R233E/E240N | | | | | |
| Y155F/R338E/T343R/ | Y[155]F/R170E/T175R/ | 3.5E+08 | 2.2E+08 | 62% | 761% | 6 |
| E410N | E240N | | | | | |
| R338E/T343R/E410N | R170E/T175R/E240N | 9.3E+07 | 2.8E+07 | 30% | 201% | 2 |
| Y155F/R318Y/T343R/ | Y[155]F/R150Y/T175R/ | 1.5E+08 | 6.6E+07 | 44% | 326% | 4 |
| E410N | E240N | | | | | |
| R318Y/T343R/E410N | R150Y/T175R/E240N | 6.2E+07 | 1.1E+07 | 17% | 135% | 2 |
| K228N/R318Y/R338E/ | K63N/R150Y/R170E/ | 2.7E+08 | 8.8E+07 | 32% | 593% | 3 |
| T343R/R403E/E410N | T175R/R233E/E240N | | | | | |
| K228N/K247N/N249S/ | K63N/K82N/N84S/R150Y/ | 2.9E+08 | 1.3E+08 | 46% | 636% | 3 |
| R318Y/R338E/T343R/ | R170E/T175R/R233E | | | | | |
| R403E | | | | | | |
| K228N/247N/N249S/ | K63N/K82N/N84S/R150Y/ | 1.3E+08 | 4.5E+07 | 35% | 278% | 2 |
| R318Y/R338E/ | R170E/T175R/E240N | | | | | |
| T343R/E410N | | | | | | |
| K228N/K247N/N249S/ | K63N/K82N/N84S/R150Y/ | 7.1E+07 | 3.3E+07 | 46% | 153% | 3 |
| R318Y/T343R/R403E/ | T175R/R233E/E240N | | | | | |
| E410N | | | | | | |

†produced in BHK-21 cells;
*80% glycosylated form of E410N

TABLE 25
Catalytic activity of FIXa variants (k_{cat})

| Mutation | Mutation | | | | |
|---|---|---|---|---|---|
| (Mature FIX | (Chymotrypsin | k_{cat} | ±S.D. | | |
| Numbering) | Numbering) | (s⁻¹) | (s⁻¹) | % CV | n |
| BeneFIX ® | BeneFIX ® | 2.8 | 1.1 | 39% | 125 |
| Coagulation FIX | Coagulation FIX | | | | |
| (T148A) | (T[148]A) | | | | |
| Plasma Purified | Plasma Purified | 3.6 | 1.2 | 34% | 120 |
| FIXa | FIXa | | | | |
| Catalyst | Catalyst | 3.1 | 1.4 | 46% | 31 |
| Biosciences WT | Biosciences WT | | | | |
| N157D | N[157]D | 3.3 | 0.5 | 16% | 2 |
| Y155F | Y[155]F | 3.7 | 0.4 | 11% | 2 |
| A103N/N105S/Y155F | A[103]N/N[105]S/ | 3.2 | 0.0 | 0% | 2 |
| | Y[155]F | | | | |
| D104N/K106S/Y155F | D[104]N/K[106]S/ | 2.9 | 0.1 | 4% | 2 |
| | Y[155]F | | | | |
| A103N/N105S | A[103]N/N[105]S | 3.1 | 1.0 | 31% | 9 |
| D104N/K106S | D[104]N/K[106]S | 3.1 | 1.1 | 34% | 9 |
| K106N/V108S | K[106]N/V[108]S | 2.5 | 0.5 | 21% | 7 |
| D85N | D[85]N | 4.2 | 0.8 | 19% | 15 |
| T148A | T[148]A | 2.2 | 0.9 | 42% | 30 |
| T148A† | T[148]A† | 1.6 | 0.2 | 14% | 7 |
| K5A | K[5]A | 3.1 | 0.2 | 8% | 2 |
| D64N | D[64]N | 1.2 | 0.4 | 31% | 2 |
| D64A | D[64]A | 0.3 | 0.2 | 70% | 2 |
| N167D | N[167]D | 2.9 | 0.8 | 27% | 2 |
| N167Q | N[167]Q | 2.3 | 0.7 | 32% | 4 |
| S61A | S[61]A | 3.6 | 1.5 | 41% | 4 |
| S53A | S[53]A | 3.7 | 1.7 | 44% | 3 |
| T159A | T[159]A | 3.7 | 1.2 | 34% | 3 |
| T169A | T[169]A | 4.6 | 1.6 | 36% | 3 |
| T172A | T[172]A | 4.4 | 1.5 | 34% | 3 |
| T179A | T[179]A | 5.1 | 0.6 | 12% | 3 |
| Y155H | Y[155]H | 4.6 | 0.9 | 18% | 3 |
| Y155Q | Y[155]Q | 4.4 | 1.0 | 24% | 3 |
| S158A | S[158]A | 3.9 | 0.1 | 3% | 2 |
| S158D | S[158]D | 3.5 | 0.3 | 8% | 2 |
| S158E | S[158]E | 3.5 | 0.2 | 5% | 2 |
| N157Q | N[157]Q | 3.5 | 0.1 | 4% | 2 |
| D203N/F205T | D39N/F41T | 1.6 | 0.6 | 40% | 12 |
| D85N/D203N/F205T | D[85]N/D39N/F41T | 1.2 | 0.5 | 40% | 5 |
| K228N | K63N | 2.7 | 1.2 | 43% | 13 |
| D85N/K228N | D[85]N/K63N | 2.7 | 0.8 | 29% | 6 |
| A103N/N105S/K228N | A[103]N/N[105]S/ | 2.1 | 0.5 | 22% | 3 |
| | K63N | | | | |
| D104N/K106S/K228N | D[104]N/K[106]S/ | 2.4 | 0.1 | 6% | 3 |
| | K63N | | | | |
| Y155F/K228N | Y[155]F/K63N | 3.3 | 0.3 | 10% | 2 |
| D104N/K106S/Y155F/ | D[104]N/K[106]S/ | 4.6 | 1.2 | 27% | 2 |
| K228N | Y[155]F/K63N | | | | |
| I251S | I86S | 3.8 | 1.1 | 30% | 13 |
| D85N/I251S | D[85]N/I86S | 2.8 | 0.6 | 22% | 5 |
| D85N/D104N/K106S/ | D[85]N/D[104]N/ | 1.5 | 0.3 | 19% | 5 |
| I251S | K[106]S/I86S | | | | |
| A103N/N105S/I251S | A[103]N/N[105]S/ | 2.9 | 1.0 | 36% | 3 |
| | I86S | | | | |
| D104N/K106S/I251S | D[104]N/K[106]S/ | 2.9 | 0.5 | 18% | 2 |
| | I86S | | | | |
| Y155F/I251S | Y[155]F/I86S | 3.7 | 0.8 | 22% | 2 |
| A262S | A95bS | 2.3 | 0.7 | 32% | 8 |
| K413N | K243N | 2.6 | 0.5 | 19% | 5 |
| E410N | E240N | 5.0 | 2.2 | 45% | 21 |
| E410N* | E240N* | 2.2 | 0.5 | 25% | 11 |
| E239N | E74N | 1.4 | 0.5 | 36% | 9 |
| T241N/H243S | T76N/H78S | 2.0 | 0.0 | 0% | 2 |
| K247N/N249S | K82N/N84S | 3.9 | 1.0 | 26% | 11 |
| Y155F/K247N/N249S | Y[155]F/K82N/N84S | 3.3 | 0.7 | 21% | 4 |
| A103N/N105S/K247N/ | A[103]N/N[105]S/ | 3.4 | 0.5 | 15% | 6 |
| N249S | K82N/N84S | | | | |
| D104N/K106S/K247N/ | D[104]N/K[106]S/ | 3.3 | 1.1 | 32% | 2 |
| N249S | K82N/N84S | | | | |
| D104N/K106S/Y155F/ | D[104]N/K[106]S/ | 2.8 | 1.1 | 40% | 3 |
| K247N/N249S | Y[155]F/K82N/N84S | | | | |
| L321N | L153N | 1.9 | 0.1 | 4% | 2 |
| F314N/H315S | F145N/H147S | No | n.d. | n.d. | 4 |
| | | Activity | | | |
| S319N/L321S | S151N/L153S | 1.4 | 0.9 | 61% | 3 |
| N260S | N95S | 1.3 | 0.5 | 42% | 13 |
| D104N/K106S/N260S | D[104]N/K[106]S/ | 1.2 | 0.7 | 58% | 2 |
| | N95S | | | | |
| Y155F/N260S | Y[155]F/N95S | 1.9 | 0.6 | 32% | 2 |
| D104N/K106S/Y155F/ | D[104]N/K[106]S/ | 0.4 | 0.1 | 28% | 2 |
| N260S | Y[155]F/N95S | | | | |
| Y284N | Y117N | 2.0 | 0.9 | 45% | 8 |
| G317N | G149N | No | n.d. | n.d. | 5 |
| | | Activity | | | |
| R318N/A320S | R150N/A152S | No | n.d. | n.d. | 8 |
| | | Activity | | | |
| R318A | R150A | 2.4 | 0.8 | 32% | 3 |
| R318E | R150E | 0.6 | 0.2 | 35% | 3 |
| R318Y | R150Y | 2.9 | 0.7 | 26% | 3 |
| R312Q | R143Q | 0.3 | 0.1 | 26% | 3 |
| R312A | R143A | 0.3 | 0.0 | 8% | 2 |
| R312Y | R143Y | 0.4 | 0.3 | 73% | 2 |
| R312L | R143L | 0.7 | 0.3 | 41% | 2 |
| V202M | V38M | 2.6 | 1.0 | 37% | 2 |
| V202Y | V38Y | 1.8 | 0.2 | 10% | 2 |
| D203M | D39M | 1.8 | 0.8 | 42% | 5 |
| D203Y | D39Y | 1.7 | 0.5 | 27% | 4 |
| A204M | A40M | 0.6 | 0.5 | 84% | 5 |
| A204Y | A40Y | 1.9 | 0.8 | 42% | 2 |
| K400A/R403A | K230A/R233A | 0.3 | 0.0 | 5% | 2 |
| K400E/R403E | K230E/R233E | No | n.d. | n.d. | 4 |
| | | Activity | | | |
| R403A | R233A | 0.6 | 0.2 | 24% | 7 |
| R403E | R233E | 0.4 | 0.1 | 25% | 6 |
| K400A | K230A | 1.4 | 0.2 | 14% | 2 |
| K400E | K230E | 0.6 | 0.0 | 4% | 2 |
| K293E | K126E | 0.5 | 0.1 | 15% | 2 |
| K293A | K126A | 1.4 | 0.4 | 28% | 2 |
| R333A | R165A | No | n.d. | n.d. | 2 |
| | | Activity | | | |
| R333E | R165E | No | n.d. | n.d. | 2 |
| | | Activity | | | |
| R338A | R170A | 5.4 | 0.3 | 5% | 2 |
| R338E | R170E | 4.7 | 1.0 | 21% | 10 |
| R338A/R403A | R170A/R233A | 3.8 | 0.9 | 24% | 6 |
| R338E/R403E | R170E/R233E | 3.3 | 1.2 | 37% | 2 |
| K293A/R403A | K126A/R233A | 0.4 | 0.0 | 9% | 2 |
| K293E/R403E | K126E/R233E | 0.1 | 0.0 | 37% | 2 |
| K293A/R338A/R403A | K126A/R170A/R233A | 1.6 | 0.7 | 41% | 2 |
| K293E/R338E/R403E | K126E/R170E/R233E | 0.8 | 0.2 | 27% | 2 |
| R318A/R403A | R150A/R233A | 0.7 | 0.1 | 12% | 2 |
| R318E/R403E | R150E/R233E | 0.1 | 0.0 | 35% | 2 |
| R318Y/E410N | R150Y/E240N | 3.5 | 0.9 | 27% | 21 |
| R338E/E410N | R170E/E240N | 5.2 | 0.8 | 16% | 8 |
| R338E/R403E/E410N | R170E/R233E/E240N | 3.3 | 1.3 | 39% | 7 |
| R318Y/R338E/R403E | R150Y/R170E/R233E | 3.5 | 0.4 | 11% | 2 |
| D203N/F205T/K228N | D39N/F41T/K63N | 0.6 | 0.2 | 27% | 2 |
| D203N/F205T/E410N | D39N/F41T/E240N | 1.7 | 0.3 | 16% | 6 |
| D203N/F205T/R338E | D39N/F41T/R170E | 2.5 | 0.0 | 2% | 2 |
| D203N/F205T/R338A | D39N/F41T/R170A | 2.3 | 0.5 | 23% | 3 |
| D203N/F205T/R318Y | D39N/F41T/R150Y | 0.9 | 0.1 | 13% | 4 |
| D203N/F205T/R338E/ | D39N/F41T/R170E/ | 0.9 | 0.0 | 5% | 2 |
| R403E | R233E | | | | |
| K228N/E410N | K63N/E240N | 3.5 | 0.9 | 27% | 10 |
| K228N/R338E | K63N/R170E | 4.8 | 0.8 | 17% | 2 |
| K228N/R338A | K63N/R170A | 6.5 | 0.5 | 7% | 2 |
| K228N/R318Y | K63N/R150Y | 2.9 | 0.6 | 19% | 5 |
| K228N/R338E/R403E | K63N/R170E/R233E | 2.8 | 0.3 | 9% | 2 |
| R403E/E410N | R233E/E240N | 2.0 | 0.2 | 9% | 2 |
| R318Y/R338E/E410N | R150Y/R170E/E240N | 4.6 | 1.3 | 29% | 26 |
| D104N/K106S/R318Y/ | D[104]N/K[106]S/ | 4.8 | 0.6 | 12% | 4 |
| R338E/E410N | R150Y/R170E/E240N | | | | |
| Y155F/R318Y/R338E/ | Y[155]F/R150Y/R170E/ | 5.6 | 1.4 | 25% | 5 |
| E410N | E240N | | | | |
| K228N/R318Y/E410N | K63N/R150Y/E240N | 5.0 | 0.5 | 10% | 4 |
| R318Y/R403E/E410N | R150Y/R233E/E240N | 2.3 | 0.3 | 15% | 3 |
| R318Y/R338E/R403E/ | R150Y/R170E/R233E/ | 5.0 | 3.1 | 63% | 14 |
| E410N | E240N | | | | |
| A103N/N105S/R318Y/ | A[103]N/N[105]S/ | 5.4 | 0.9 | 16% | 5 |
| R338E/R403E/E410N | R150Y/R170E/R233E/ | | | | |
| | E240N | | | | |
| D104N/K106S/R318Y/ | D[104]N/K[106]S/ | 5.7 | 1.1 | 20% | 3 |
| R338E/R403E/E410N | R150Y/R170E/R233E/ | | | | |
| | E240N | | | | |
| Y155F/R318Y/R338E/ | Y[155]F/R150Y/R170E/ | 5.3 | 0.7 | 12% | 4 |
| R403E/E410N | R233E/E240N | | | | |
| A103N/N105S/Y155F/ | A[103]N/N[105]S/ | 6.4 | 0.5 | 7% | 2 |
| R318Y/R338E/R403E/ | Y[155]F/R150Y/R170E/ | | | | |
| E410N | R233E/E240N | | | | |
| D104N/K106S/Y155F/ | D[104]N/K[106]S/ | 8.5 | 0.8 | 10% | 2 |
| R318Y/R338E/R403E/ | Y[155]F/R150Y/R170E/ | | | | |
| E410N | R233E/E240N | | | | |
| D203N/F205T/R318Y/ | D39N/F41T/R150Y/ | 1.6 | 0.6 | 36% | 6 |
| E410N | E240N | | | | |
| R333S | R165S | 0.05 | 0.01 | 22% | 3 |
| R338L | R170L | 9.5 | 1.9 | 21% | 3 |
| K316N | K148N | 0.3 | 0.1 | 39% | 3 |
| K316A | K148A | 0.3 | 0.1 | 21% | 3 |
| K316E | K148E | 0.1 | 0.0 | 9% | 3 |
| K316S | K148S | 0.2 | 0.0 | 10% | 3 |
| K316M | K148M | 0.7 | 0.1 | 15% | 3 |
| E239S | E74S | 0.7 | 0.1 | 19% | 3 |
| E239A | E74A | 2.8 | 1.2 | 43% | 3 |
| E239R | E74R | 3.4 | 1.4 | 42% | 3 |
| E239K | E74K | 3.0 | 1.1 | 36% | 3 |
| H257F | H92F | 3.0 | 1.4 | 46% | 3 |
| H257Y | H92Y | 2.0 | 1.1 | 55% | 3 |
| H257E | H92E | 1.3 | 0.4 | 28% | 3 |
| H257S | H92S | 1.8 | 0.3 | 18% | 3 |
| T412A | T242A | 2.6 | 0.3 | 13% | 5 |
| T412V | T242V | 2.6 | 0.6 | 25% | 8 |
| E410N/T412A | E240N/T242A | 2.9 | 0.4 | 13% | 4 |
| E410N/T412V | E240N/T242V | 2.9 | 0.5 | 16% | 4 |
| E410Q | E240Q | 6.0 | 2.8 | 46% | 4 |
| E410S | E240S | 4.9 | 1.6 | 32% | 12 |
| E410A | E240A | 4.8 | 1.6 | 32% | 10 |
| E410D | E240D | 4.0 | 0.7 | 19% | 4 |
| N346D | N178D | 0.8 | 0.2 | 29% | 4 |
| Y155F/N346D | Y[155]F/N178D | 1.3 | 0.5 | 41% | 2 |
| N346Y | N178Y | 2.6 | 0.2 | 9% | 8 |
| Y345A | Y177A | 0.7 | 0.6 | 83% | 4 |
| Y345T | Y177T | 1.3 | 0.3 | 27% | 4 |
| T343R | T175R | 4.3 | 1.2 | 27% | 9 |
| T343E | T175E | 1.0 | 0.7 | 72% | 4 |
| T343Q | T175Q | 2.5 | 0.3 | 11% | 3 |
| F342I | F174I | 1.3 | 0.2 | 16% | 3 |
| T343R/Y345T | TI75R/YI77T | 2.4 | 0.3 | 14% | 3 |
| R318Y/R338E | R150Y/R170E | 3.4 | 0.5 | 14% | 4 |
| Y259F/K265T/Y345T | Y94F/K98T/Y177T | 1.7 | 0.1 | 5% | 2 |
| K228N/I251S | K63N/I86S | 2.7 | 1.1 | 41% | 2 |
| K228N/R318Y/R338E/ | K63N/R150Y/R170E/ | 5.1 | 0.7 | 14% | 3 |
| R403E/E410N | R233E/E240N | | | | |
| Y155F/K228N/R318Y/ | Y[155]F/K63N/R150Y/ | 9.7 | 1.6 | 16% | 2 |
| R338E/R403E/E410N | R170E/R233E/E240N | | | | |
| D85N/K228N/R318Y/ | D[85]N/K63N/R150Y/ | 6.0 | 0.6 | 10% | 2 |
| R338E/R403E/E410N | R170E/R233E/E240N | | | | |
| I251S/R318Y/R338E/ | I86S/R150Y/R170E/ | 4.8 | 0.6 | 12% | 4 |
| R403E/E410N | R233E/E240N | | | | |
| D104N/K106S/I251S/ | D[104]N/K[106]S/ | 5.5 | 0.9 | 17% | 8 |
| R318Y/R338E/R403E/ | I86S/R150Y/R170E/ | | | | |
| E410N | R233E/E240N | | | | |
| Y155F/I251S/R318Y/ | Y[155]F/I86S/ | 7.2 | 0.8 | 11% | 2 |
| R338E/R403E/E410N | R150Y/R170E/R233E/ | | | | |
| | E240N | | | | |
| I251S/R318Y/R338E/ | I86S/R150Y/R170E/ | 6.2 | 1.2 | 20% | 7 |
| E410N | E240N | | | | |
| D104N/K106S/I251S/ | D[104]N/K[106]S/ | 3.1 | 0.6 | 19% | 3 |
| R318Y/R338E/E410N | I86S/R150Y/R170E/ | | | | |
| | E240N | | | | |
| F314N/K316S | F145N/K148S | 0.0 | 0.0 | 7% | 2 |
| K247N/N249S/R318Y/ | K82N/N84S/R150Y/ | 5.8 | 1.1 | 19% | 6 |
| R338E/R403E/E410N | R170E/R233E/E240N | | | | |
| Y155F/K247N/N249S/ | Y[155]F/K82N/N84S/ | 6.5 | 1.1 | 17% | 6 |
| R318Y/R338E/R403E/ | R150Y/R170E/R233E/ | | | | |
| E410N | E240N | | | | |
| A103N/N105S/K247N/ | A[103]N/N[105]S/ | 4.1 | 0.8 | 18% | 2 |
| N249S/R318Y/R338E/ | K82N/N84S/R150Y/ | | | | |
| R403E/E410N | R170E/R233E/E240N | | | | |
| D104N/K106S/K247N/ | D[104]N/K[106]S/ | 5.2 | 0.3 | 6% | 2 |
| N249S/R318Y/R338E/ | K82N/N84S/R150Y/ | | | | |
| R403E/E410N | R170E/R233E/E240N | | | | |
| K247N/N249S/R318Y/ | K82N/N84S/R150Y/ | 3.8 | 1.6 | 41% | 6 |
| R338E/E410N | R170E/E240N | | | | |
| Y155F/K247N/N249S/ | Y[155]F/K82N/N84S/ | 4.3 | 1.4 | 33% | 7 |
| R318Y/R338E/E410N | R150Y/R170E/E240N | | | | |
| R318Y/R338E/R403E/ | R150Y/R170E/R233E/ | 5.8 | 0.6 | 10% | 4 |
| E410S | E240S | | | | |
| R318Y/R338E/E410S | R150Y/R170E/E240S | 5.1 | 1.7 | 33% | 8 |
| K228N/K247N/N249S | K63N/K82N/N84S | 3.5 | 0.1 | 4% | 2 |
| D104N/K106S/Y155F/ | D[104]N/K[106]S/ | 4.7 | 1.4 | 30% | 2 |
| K228N/K247N/N249S | Y[155]F/K63N/K82N/ | | | | |
| | N84S | | | | |
| D104N/K106S/K228N/ | D[104]N/K[106]S/ | 1.7 | 0.9 | 54% | 5 |
| K247N/N249S | K63N/K82N/N84S | | | | |
| Y155F/K228N/K247N/ | Y[155]F/K63N/K82N/ | 4.3 | 1.9 | 44% | 2 |
| N249S | N84S | | | | |
| K228N/K247N/N249S/ | K63N/K82N/N84S/ | 6.1 | 0.7 | 12% | 3 |
| R318Y/R338E/R403E/ | R150Y/R170E/R233E/ | | | | |
| E410N | E240N | | | | |
| R318Y/R338E/R403E/ | R150Y/R170E/R233E/ | 7.9 | 2.1 | 26% | 4 |
| E410N/T412V | E240N/T242V | | | | |
| R318Y/R338E/R403E/ | R150Y/R170E/R233E/ | 8.4 | 1.5 | 18% | 4 |
| E410N/T412A | E240N/T242A | | | | |
| R318Y/R338E/R403E/ | R150Y/R170E/R233E/ | 5.1 | 1.1 | 21% | 4 |
| T412A | T242A | | | | |
| R318Y/R338E/T412A | R150Y/R170E/T242A | 7.0 | 2.8 | 39% | 6 |
| R318Y/R338E/E410N/ | R150Y/R170E/E240N/ | 6.3 | 2.3 | 37% | 4 |
| T412V | T242V | | | | |
| N260S/R318Y/R338E/ | N95S/R150Y/R170E/ | 3.8 | 1.1 | 29% | 2 |
| R403E/E410N | R233E/E240N | | | | |
| D104N/K106S/N260S/ | D[104]N/K[106]S/ | 5.4 | 0.5 | 9% | 2 |
| R318Y/R338E/R403E/ | N95S/R150Y/R170E/ | | | | |
| E410N | R233E/E240N | | | | |
| Y155F/N260S/R318Y/ | Y[155]F/N95S/R150Y/ | 5.8 | 1.7 | 30% | 2 |
| R338E/R403E/E410N | R170E/R233E/E240N | | | | |
| R318Y/R338E/N346D/ | R150Y/R170E/N178D/ | 2.5 | 1.3 | 54% | 2 |
| R403E/E410N | R233E/E240N | | | | |
| Y155F/R318Y/R338E/ | Y[155]F/R150Y/R170E/ | 6.4 | 2.8 | 44% | 2 |
| N346D/R403E/E410N | N178D/R233E/E240N | | | | |
| K247N/N249S/N260S | K82N/N84S/N95S | 3.3 | 0.3 | 9% | 2 |
| Y155F/K247N/N249S/ | Y[155]F/K82N/N84S/ | 1.8 | 0.3 | 16% | 2 |
| N260S | N95S | | | | |
| D104N/K106S/K247N/ | D[104]N/K[106]S/ | 0.6 | 0.0 | 7% | 2 |
| N249S/N260S | K82N/N84S/N95S | | | | |
| D104N/K106S/Y155F/ | D[104]N/K[106]S/ | 0.5 | 0.0 | 2% | 2 |
| K247N/N249S/N260S | Y[155]F/K82N/N84S/ | | | | |
| | N95S | | | | |
| K247N/N249S/N260S/ | K82N/N84S/N95S/ | 6.0 | 0.5 | 9% | 2 |
| R318Y/R338E/R403E/ | R150Y/R170E/R233E/ | | | | |
| E410N | E240N | | | | |
| Y155F/N260S/N346D | Y[155]F/N95S/N178D | 0.3 | 0.1 | 29% | 2 |
| R318Y/R338E/T343R/ | R150Y/R170E/T175R/ | 11.8 | 2.4 | 20% | 3 |
| R403E/E410N | R233E/E240N | | | | |
| R338E/T343R | R170E/T175R | 7.7 | 1.3 | 17% | 4 |

†produced in BHK-21 cells;
*80% glycosylated form of E410N

TABLE 26
Catalytic activity of FIXa variants (k_{cat})

| Mutation | Mutation | | | | |
|---|---|---|---|---|---|
| (Mature FIX | (Chymotrypsin | k_{cat} | ± S.D. | | |
| Numbering) | Numbering) | (s⁻¹) | (s⁻¹) | % CV | n |
| BeneFIX ® | BeneFIX ® | 2.9 | 1.1 | 39% | 140 |
| Coagulation FIX | Coagulation FIX | | | | |
| (T148A) | (T[148]A) | | | | |
| Plasma Purified | Plasma Purified | 3.7 | 1.3 | 36% | 200 |
| FIXa | FIXa | | | | |
| Catalyst | Catalyst | 3.1 | 1.4 | 46% | 33 |
| Biosciences WT | Biosciences WT | | | | |
| N157D | N[157]D | 3.3 | 0.5 | 16% | 2 |
| Y155F | Y[155]F | 3.7 | 0.4 | 11% | 2 |
| A103N/N105S/Y155F | A[103]N/N[105]S/ | 3.2 | 0.0 | 0% | 2 |
| | Y[155]F | | | | |
| D104N/K106S/Y155F | D[104]N/K[106]S/ | 2.9 | 0.1 | 4% | 2 |
| | Y[155]F | | | | |
| A103N/N105S | A[103]N/N[105]S | 3.1 | 1.0 | 31% | 9 |
| D104N/K106S | D[104]N/K[106]S | 3.1 | 1.1 | 34% | 9 |
| K106N/V108S | K[106]N/V[108]S | 2.5 | 0.5 | 21% | 7 |
| D85N | D[85]N | 4.1 | 0.8 | 20% | 17 |
| T148A | T[148]A | 2.5 | 1.0 | 39% | 44 |
| T148A† | T[148]A† | 1.6 | 0.2 | 14% | 7 |
| K5A | K[5]A | 3.5 | 0.8 | 23% | 4 |
| D64N | D[64]N | 1.2 | 0.4 | 31% | 2 |
| D64A | D[64]A | 0.3 | 0.2 | 70% | 2 |
| N167D | N[167]D | 2.9 | 0.8 | 27% | 2 |
| N167Q | N[167]Q | 2.3 | 0.7 | 32% | 4 |
| S61A | S[61]A | 3.6 | 1.5 | 41% | 4 |
| S53A | S[531A | 3.7 | 1.7 | 44% | 3 |
| T159A | T[159]A | 3.7 | 1.2 | 34% | 3 |
| T169A | T[169]A | 4.6 | 1.6 | 36% | 3 |
| T172A | T[172]A | 4.4 | 1.5 | 34% | 3 |
| T179A | T[179]A | 5.1 | 0.6 | 12% | 3 |
| Y155H | Y[155]H | 4.6 | 0.9 | 18% | 3 |
| Y155Q | Y[155]Q | 4.4 | 1.0 | 24% | 3 |
| S158A | S[158]A | 3.9 | 0.1 | 3% | 2 |
| S158D | S[158]D | 3.5 | 0.3 | 8% | 2 |
| S158E | S[158]E | 3.5 | 0.2 | 5% | 2 |
| N157Q | N[157]Q | 3.5 | 0.1 | 4% | 2 |
| D203N/F205T | D39N/F41T | 1.6 | 0.6 | 40% | 12 |
| D85N/D203N/F205T | D[85]N/D39N/F41T | 1.2 | 0.5 | 40% | 5 |
| K228N | K63N | 2.7 | 1.2 | 43% | 13 |
| D85N/K228N | D[85]N/K63N | 2.7 | 0.8 | 29% | 6 |
| A103N/N105S/K228N | A[103]N/N[105]S/ | 2.1 | 0.5 | 22% | 3 |
| | K63N | | | | |
| D104N/K106S/K228N | D[104]N/K[106]S/ | 2.4 | 0.1 | 6% | 3 |
| | K63N | | | | |
| Y155F/K228N | Y[155]F/K63N | 3.3 | 0.3 | 10% | 2 |
| D104N/K106S/Y155F/ | D[104]N/K[106]S/ | 4.6 | 1.2 | 27% | 2 |
| K228N | Y[155]F/K63N | | | | |
| I251S | I86S | 3.8 | 1.1 | 30% | 13 |
| D85N/I251S | D[85]N/I86S | 2.8 | 0.6 | 22% | 5 |
| D85N/D104N/K106S/ | D[85]N/D[104]N/ | 1.5 | 0.3 | 19% | 5 |
| I251S | K[106]S/I86S | | | | |
| A103N/N105S/I251S | A[103]N/N[105]S/ | 2.9 | 1.0 | 36% | 3 |
| | I86S | | | | |
| D104N/K106S/I251S | D[104]N/K[106]S/ | 2.9 | 0.5 | 18% | 2 |
| | I86S | | | | |
| Y155F/I251S | Y[155]F/I86S | 3.7 | 0.8 | 22% | 2 |
| A262S | A95bS | 2.3 | 0.7 | 32% | 8 |
| K413N | K243N | 2.5 | 0.5 | 19% | 7 |
| E410N | E240N | 4.9 | 2.0 | 41% | 27 |
| E410N* | E240N* | 2.3 | 0.5 | 22% | 10 |
| E239N | E74N | 1.4 | 0.5 | 36% | 9 |
| T241N/H243S | T76N/H78S | 2.0 | 0.0 | 0% | 2 |
| K247N/N249S | K82N/N84S | 3.9 | 1.0 | 26% | 11 |
| Y155F/K247N/N249S | Y[155]F/K82N/N84S | 3.3 | 0.7 | 21% | 4 |
| A103N/N105S/K247N/ | A[103]N/N[105]S/ | 3.4 | 0.5 | 15% | 6 |
| N249S | K82N/N84S | | | | |
| D104N/K106S/K247N/ | D[104]N/K[106]S/ | 3.3 | 1.1 | 32% | 2 |
| N249S | K82N/N84S | | | | |
| D104N/K106S/Y155F/ | D[104]N/K[106]S/ | 2.8 | 1.1 | 40% | 3 |
| K247N/N249S | Y[155]F/K82N/N84S | | | | |
| L321N | L153N | 1.9 | 0.1 | 4% | 2 |
| F314N/H315S | F145N/H147S | 0.0 | 0.0 | 7% | 2 |
| K392N/K394S | K222N/K224S | 0.0 | n.d. | n.d. | 0 |
| S319N/L321S | S151N/L153S | 1.4 | 0.9 | 61% | 3 |
| N260S | N95S | 1.3 | 0.5 | 42% | 13 |
| D104N/K106S/N260S | D[104]N/K[106]S/ | 1.2 | 0.7 | 58% | 2 |
| | N95S | | | | |
| Y155F/N260S | Y[155]F/N95S | 1.9 | 0.6 | 32% | 2 |
| D104N/K106S/Y155F/ | D[104]N/K[106]S/ | 0.4 | 0.1 | 28% | 2 |
| N260S | Y[155]F/N95S | | | | |
| Y284N | Y117N | 2.0 | 0.9 | 45% | 8 |
| G317N | G149N | 0.0 | n.d. | n.d. | 1 |
| R318N/A320S | R150N/A152S | 0.0 | 0.0 | 95% | 3 |
| R318A | R150A | 2.7 | 0.9 | 32% | 2 |
| R318E | R150E | 0.6 | 0.2 | 35% | 3 |
| R318Y | R150Y | 2.9 | 0.7 | 26% | 3 |
| R312Q | R143Q | 0.3 | 0.1 | 26% | 3 |
| R312A | R143A | 0.3 | 0.0 | 8% | 2 |
| R312Y | R143Y | 0.4 | 0.3 | 73% | 2 |
| R312L | R143L | 0.7 | 0.3 | 41% | 2 |
| V202M | V38M | 2.6 | 1.0 | 37% | 2 |
| V202Y | V38Y | 1.8 | 0.2 | 10% | 2 |
| D203M | D39M | 1.8 | 0.8 | 42% | 5 |
| D203Y | D39Y | 1.7 | 0.5 | 27% | 4 |
| A204M | A40M | 0.6 | 0.5 | 84% | 5 |
| A204Y | A40Y | 1.9 | 0.8 | 42% | 2 |
| K400A/R403A | K230A/R233A | 0.3 | 0.0 | 5% | 2 |
| K400E/R403E | K230E/R233E | 0.1 | 0.0 | 50% | 3 |
| R403A | R233A | 0.6 | 0.2 | 24% | 7 |
| R403E | R233E | 0.4 | 0.1 | 25% | 6 |
| K400A | K230A | 1.4 | 0.2 | 14% | 2 |
| K400E | K230E | 0.6 | 0.0 | 4% | 2 |
| K293E | K126E | 0.5 | 0.1 | 15% | 2 |
| K293A | K126A | 1.4 | 0.4 | 28% | 2 |
| R333A | R165A | 0.1 | 0.0 | 35% | 2 |
| R333E | R165E | 0.0 | n.d. | n.d. | 1 |
| R338A | R170A | 5.4 | 0.3 | 5% | 2 |
| R338E | R170E | 4.7 | 1.0 | 21% | 10 |
| R338A/R403A | R170A/R233A | 3.8 | 0.9 | 24% | 6 |
| R338E/R403E | R170E/R233E | 3.3 | 1.2 | 37% | 2 |
| K293A/R403A | K126A/R233A | 0.4 | 0.0 | 9% | 2 |
| K293E/R403E | K126E/R233E | 0.1 | 0.0 | 37% | 2 |
| K293A/R338A/R403A | K126A/R170A/R233A | 1.6 | 0.7 | 41% | 2 |
| K293E/R338E/R403E | K126E/R170E/R233E | 0.8 | 0.2 | 27% | 2 |
| R318A/R403A | R150A/R233A | 0.7 | 0.1 | 12% | 2 |
| R318E/R403E | R150E/R233E | 0.1 | 0.0 | 35% | 2 |
| R318Y/E410N | R150Y/E240N | 3.5 | 0.9 | 27% | 21 |
| R338E/E410N | R170E/E240N | 5.2 | 1.1 | 22% | 12 |
| R338E/R403E/E410N | R170E/R233E/E240N | 5.8 | 3.0 | 52% | 17 |
| Y155F/R338E/R403E/ | Y[155]F/R170E/R233E/ | 5.9 | 0.4 | 7% | 2 |
| E410N | E240N | | | | |
| R318Y/R338E/R403E | R150Y/R170E/R233E | 3.6 | 0.4 | 10% | 3 |
| Y155F/R318Y/R338E/ | Y[155]F/R150Y/R170E/ | 5.1 | 1.0 | 19% | 2 |
| R403E | R233E | | | | |
| D203N/F205T/K228N | D39N/F41T/K63N | 0.6 | 0.2 | 27% | 2 |
| D203N/F205T/E410N | D39N/F41T/E240N | 1.7 | 0.3 | 16% | 6 |
| D203N/F205T/R338E | D39N/F41T/R170E | 2.5 | 0.0 | 2% | 2 |
| D203N/F205T/R338A | D39N/F41T/R170A | 2.3 | 0.5 | 23% | 3 |
| D203N/F205T/R318Y | D39N/F41T/R150Y | 0.9 | 0.1 | 13% | 4 |
| D203N/F205T/R338E/ | D39N/F41T/R170E/ | 0.9 | 0.0 | 5% | 2 |
| R403E | R233E | | | | |
| K228N/E410N | K63N/E240N | 3.5 | 0.9 | 27% | 10 |
| K228N/R338E | K63N/R170E | 4.8 | 0.8 | 17% | 2 |
| K228N/R338A | K63N/R170A | 6.5 | 0.5 | 7% | 2 |
| K228N/R318Y | K63N/R150Y | 2.9 | 0.6 | 19% | 5 |
| K228N/R338E/R403E | K63N/R170E/R233E | 2.8 | 0.3 | 9% | 2 |
| R403E/E410N | R233E/E240N | 2.0 | 0.2 | 9% | 2 |
| R318Y/R338E/E410N | R150Y/R170E/E240N | 4.4 | 1.2 | 27% | 42 |
| D104N/K106S/R318Y/ | D[104]N/K[106]S/ | 4.8 | 0.6 | 12% | 4 |
| R338E/E410N | R150Y/R170E/E240N | | | | |
| Y155F/R318Y/R338E/ | Y[155]F/R150Y/ | 5.6 | 1.4 | 25% | 5 |
| E410N | R170E/E240N | | | | |
| K228N/R318Y/E410N | K63N/R150Y/E240N | 5.0 | 0.5 | 10% | 4 |
| R318Y/R403E/E410N | R150Y/R233E/E240N | 2.3 | 0.3 | 11% | 5 |
| Υ155F/R318Y/R403E/ | Y[155]F/R150Y/R233E/ | 2.9 | 0.6 | 20% | 2 |
| E410N | E240N | | | | |
| R318Y/R338E/R403E/ | R150Y/R170E/R233E/ | 5.8 | 2.8 | 48% | 26 |
| E410N | E240N | | | | |
| A103N/N105S/R318Y/ | A[103]N/N[105]S/ | 5.4 | 0.9 | 16% | 5 |
| R338E/R403E/E410N | R150Y/R170E/R233E/ | | | | |
| | E240N | | | | |
| D104N/K106S/R318Y/ | D[104]N/K[106]S/ | 5.7 | 1.1 | 20% | 3 |
| R338E/R403E/E410N | R150Y/R170E/R233E/ | | | | |
| | E240N | | | | |
| Y155F/R318Y/R338E/ | Y[155]F/R150Y/R170E/ | 5.3 | 0.7 | 12% | 4 |
| R403E/E410N | R233E/E240N | | | | |
| A103N/N105S/Y155F/ | A[103]N/N[105]S/ | 6.4 | 0.5 | 7% | 2 |
| R318Y/R338E/R403E/ | Y[155]F/R150Y/R170E/ | | | | |
| E410N | R233E/E240N | | | | |
| D104N/K106S/Y155F/ | D[104]N/K[106]S/ | 8.5 | 0.8 | 10% | 2 |
| R318Y/R338E/R403E/ | Y[155]F/R150Y/R170E/ | | | | |
| E410N | R233E/E240N | | | | |
| D203N/F205T/R318Y/ | D39N/F41T/R150Y/ | 1.6 | 0.6 | 36% | 6 |
| E410N | E240N | | | | |
| R333S | R165S | 0.1 | 0.0 | 22% | 3 |
| R338L | R170L | 9.5 | 1.9 | 21% | 3 |
| K316N | K148N | 0.3 | 0.1 | 39% | 3 |
| K316A | K148A | 0.3 | 0.1 | 21% | 3 |
| K316E | K148E | 0.1 | 0.0 | 9% | 3 |
| K316S | K148S | 0.2 | 0.0 | 10% | 3 |
| K316M | K148M | 0.7 | 0.1 | 15% | 3 |
| E239S | E74S | 0.7 | 0.1 | 19% | 3 |
| E239A | E74A | 2.8 | 1.2 | 43% | 3 |
| E239R | E74R | 3.4 | 1.4 | 42% | 3 |
| E239K | E74K | 3.0 | 1.1 | 36% | 3 |
| H257F | H92F | 3.0 | 1.4 | 46% | 3 |
| H257Y | H92Y | 2.0 | 1.1 | 55% | 3 |
| H257E | H92E | 1.3 | 0.4 | 28% | 3 |
| H257S | H92S | 1.8 | 0.3 | 18% | 3 |
| T412A | T242A | 2.6 | 0.3 | 13% | 5 |
| T412V | T242V | 2.6 | 0.6 | 25% | 8 |
| E410N/T412A | E240N/T242A | 2.9 | 0.4 | 13% | 4 |
| E410N/T412V | E240N/T242V | 2.9 | 0.5 | 16% | 4 |
| E410Q | E240Q | 6.0 | 2.8 | 46% | 4 |
| E410S | E240S | 4.9 | 1.6 | 32% | 12 |
| E410A | E240A | 4.8 | 1.6 | 32% | 10 |
| E410D | E240D | 4.0 | 0.7 | 19% | 4 |
| N346D | N178D | 0.8 | 0.2 | 29% | 4 |
| Y155F/N346D | Y[155]F/N178D | 1.3 | 0.5 | 41% | 2 |
| N346Y | N178Y | 2.6 | 0.2 | 9% | 8 |
| Y345A | Y177A | 0.7 | 0.6 | 83% | 4 |
| Y345T | Y177T | 1.3 | 0.3 | 27% | 4 |
| T343R | T175R | 4.1 | 1.1 | 27% | 12 |
| T343E | T175E | 1.0 | 0.7 | 72% | 4 |
| T343Q | T175Q | 2.5 | 0.3 | 11% | 3 |
| F342I | F174I | 1.3 | 0.2 | 16% | 3 |
| T343R/Y345T | T175R/Y177T | 2.4 | 0.3 | 14% | 3 |
| R318Y/R338E | R150Y/R170E | 3.4 | 0.5 | 14% | 4 |
| Y259F/K265T/Y345T | Y94F/K98T/Y177T | 1.7 | 0.1 | 5% | 2 |
| K228N/I251S | K63N/I86S | 2.7 | 1.1 | 41% | 2 |
| K228N/R318Y/R338E/ | K63N/R150Y/R170E/ | 5.1 | 0.7 | 14% | 3 |
| R403E/E410N | R233E/E240N | | | | |
| Y155F/K228N/R318Y/ | Y[155]F/K63N/R150Y/ | 6.7 | 3.0 | 45% | 5 |
| R338E/R403E/E410N | R170E/R233E/E240N | | | | |
| D85N/K228N/R318Y/ | D[85]N/K63N/R150Y/ | 6.0 | 0.6 | 10% | 2 |
| R338E/R403E/E410N | R170E/R233E/E240N | | | | |
| I251S/R318Y/R338E/ | I86S/R150Y/R170E/ | 4.8 | 0.6 | 12% | 4 |
| R403E/E410N | R233E/E240N | | | | |
| D104N/K106S/I251S/ | D[104]N/K[106]S/ | 5.5 | 0.9 | 17% | 8 |
| R318Y/R338E/R403E/ | I86S/R150Y/R170E/ | | | | |
| E410N | R233E/E240N | | | | |
| Y155F/I251S/R318Y/ | D[104]N/K[106]S/ | 7.2 | 0.8 | 11% | 2 |
| R338E/R403E/E410N | I86S/R150Y/R170E/ | | | | |
| | R233E/E240N | | | | |
| I251S/R318Y/R338E/ | I86S/R150Y/R170E/ | 6.4 | 2.0 | 31% | 10 |
| E410N | E240N | | | | |
| D104N/K106S/I251S/ | D[104]N/K[106]S/ | 3.1 | 0.6 | 19% | 3 |
| R318Y/R338E/E410N | I86S/R150Y/R170E/ | | | | |
| | E240N | | | | |
| F314N/K316S | F145N/K148S | 0.0 | 0.0 | 7% | 2 |
| K247N/N249S/R318Y/ | K82N/N84S/R150Y/ | 5.8 | 1.1 | 19% | 6 |
| R338E/R403E/E410N | R170E/R233E/E240N | | | | |
| Y155F/K247N/N249S/ | Y[155]F/K82N/N84S/ | 6.2 | 1.0 | 16% | 10 |
| R318Y/R338E/R403E/ | R150Y/R170E/R233E/ | | | | |
| E410N | E240N | | | | |
| A103N/N105S/K247N/ | A[103]N/N[105]S/ | 3.9 | 0.4 | 11% | 6 |
| N249S/R318Y/R338E/ | K82N/N84S/R150Y/ | | | | |
| R403E/E410N | R170E/R233E/E240N | | | | |
| D104N/K106S/K247N/ | D[104]N/K[106]S/ | 5.2 | 0.3 | 6% | 2 |
| N249S/R318Y/R338E/ | K82N/N84S/R150Y/ | | | | |
| R403E/E410N | R170E/R233E/E240N | | | | |
| D104N/K106S/Y155F/ | D[104]N/K[106]S/ | 6.9 | 4.7 | 67% | 6 |
| K247N/N249S/R318Y/ | Y[155]F/K82N/N84S/ | | | | |
| R338E/R403E/E410N | R150Y/R170E/R233E/ | | | | |
| | E240N | | | | |
| K247N/N249S/R318Y/ | K82N/N84S/R150Y/ | 3.8 | 1.6 | 41% | 6 |
| R338E/E410N | R170E/E240N | | | | |
| Y155F/K247N/N249S/ | Y[155]F/K82N/N84S/ | 4.5 | 1.3 | 28% | 9 |
| R318Y/R338E/E410N | R150Y/R170E/E240N | | | | |
| R318Y/R338E/R403E/ | R150Y/R170E/R233E/ | 7.4 | 2.3 | 31% | 7 |
| E410S | E240S | | | | |
| R318Y/R338E/E410S | R150Y/R170E/E240S | 5.1 | 1.7 | 33% | 8 |
| K228N/K247N/N249S | K63N/K82N/N84S | 3.5 | 0.1 | 4% | 2 |
| D104N/K106S/Y155F/ | D[104]N/K[106]S/ | 4.7 | 1.4 | 30% | 2 |
| K228N/K247N/N249S | Y[155]F/K63N/K82N/ | | | | |
| | N84S | | | | |
| D104N/K106S/K228N/ | D[104]N/K[106]S/ | 1.7 | 0.9 | 54% | 5 |
| K247N/N249S | K63N/K82N/N84S | | | | |
| Y155F/K228N/K247N/ | Y[155]F/K63N/K82N/ | 4.3 | 1.9 | 44% | 2 |
| N249S | N84S | | | | |
| K228N/K247N/N249S/ | K63N/K82N/N84S/ | 7.1 | 2.2 | 31% | 17 |
| R318Y/R338E/R403E/ | R150Y/R170E/R233E/ | | | | |
| E410N | E240N | | | | |
| D104N/K106S/K228N/ | D[104]N/K[106]S/ | 6.1 | 3.7 | 61% | 7 |
| K247N/N249S/R318Y/ | K63N/K82N/N84S/ | | | | |
| R338E/R403E/E410N | R150Y/R170E/R233E/ | | | | |
| | E240N | | | | |
| Y155F/K228N/K247N/ | Y[155]F/K63N/K82N/ | 5.1 | 1.8 | 34% | 5 |
| N249S/R318Y/R338E/ | N84S/R150Y/R170E/ | | | | |
| R403E/E410N | R233E/E240N | | | | |
| R318Y/R338E/R403E/ | R150Y/R170E/R233E/ | 7.0 | 2.1 | 30% | 6 |
| E410N/T412V | E240N/T242V | | | | |
| R318Y/R338E/R403E/ | R150Y/R170E/R233E/ | 7.8 | 1.6 | 20% | 6 |
| E410N/T412A | E240N/T242A | | | | |
| R318Y/R338E/R403E/ | R150Y/R170E/R233E/ | 5.1 | 1.1 | 21% | 4 |
| T412A | T242A | | | | |
| R318Y/R338E/T412A | R150Y/R170E/T242A | 7.0 | 2.8 | 39% | 6 |
| R318Y/R338E/E410N/ | R150Y/R170E/E240N/ | 5.2 | 1.7 | 33% | 11 |
| T412V | T242V | | | | |
| N260S/R318Y/R338E/ | N95S/R150Y/R170E/ | 3.8 | 1.1 | 29% | 2 |
| R403E/E410N | R233E/E240N | | | | |
| D104N/K106S/N260S/ | D[104]N/K[106]S/ | 5.4 | 0.5 | 9% | 2 |
| R318Y/R338E/R403E/ | N95S/R150Y/R170E/ | | | | |
| E410N | R233E/E240N | | | | |
| Y155F/N260S/R318Y/ | Y[155]F/N95S/R150Y/ | 5.8 | 1.7 | 30% | 2 |
| R338E/R403E/E410N | R170E/R233E/E240N | | | | |
| R318Y/R338E/N346D/ | R150Y/R170E/N178D/ | 2.5 | 1.3 | 54% | 2 |
| R403E/E410N | R233E/E240N | | | | |
| Y155F/R318Y/R338E/ | Y[155]F/R150Y/R170E/ | 6.4 | 2.8 | 44% | 2 |
| N346D/R403E/E410N | N178D/R233E/E240N | | | | |
| K247N/N249S/N260S | K82N/N84S/N95S | 3.3 | 0.3 | 9% | 2 |
| Y155F/K247N/N249S/ | Y[155]F/K82N/N84S/ | 1.8 | 0.3 | 16% | 2 |
| N260S | N95S | | | | |
| D104N/K106S/K247N/ | D[104]N/K[106]S/ | 0.6 | 0.0 | 7% | 2 |
| N249S/N260S | K82N/N84S/N95S | | | | |
| D104N/K106S/Y155F/ | D[104]N/K[106]S/ | 0.5 | 0.0 | 2% | 2 |
| K247N/N249S/N260S | Y[155]F/K82N/N84S/ | | | | |
| | N95S | | | | |
| K247N/N249S/N260S/ | K82N/N84S/N95S/ | 3.4 | 2.1 | 62% | 6 |
| R318Y/R338E/R403E/ | R150Y/R170E/R233E/ | | | | |
| E410N | E240N | | | | |
| Y155F/K247N/N249S/ | Y[155]F/K82N/N84S/ | 3.6 | 1.2 | 33% | 5 |
| N260S/R318Y/R338E/ | N95S/R150Y/R170E/ | | | | |
| R403E/E410N | R233E/E240N | | | | |
| Y155F/N260S/N346D | Y[155]F/N95S/N178D | 0.3 | 0.1 | 29% | 2 |
| R318Y/R338E/T343R/ | R150Y/R170E/T175R/ | 9.7 | 2.6 | 27% | 13 |
| R403E/E410N | R233E/E240N | | | | |
| Y155F/R318Y/R338E/ | Y[155]F/R150Y/R170E/ | 7.8 | 1.9 | 24% | 4 |
| T343R/R403E/E410N | T175R/R233E/E240N | | | | |
| D104N/K106S/R318Y/ | D[104]N/K[106]S/ | 5.7 | 2.3 | 41% | 5 |
| R338E/T343R/R403E/ | R150Y/R170E/T175R/ | | | | |
| E410N | R233E/E240N | | | | |
| R338E/T343R | R170E/T175R | 7.1 | 1.4 | 20% | 7 |
| T343R/N346Y | T175R/N178Y | 2.3 | 0.5 | 21% | 11 |
| R318Y/R338E/N346Y/ | R150Y/R170E/N178Y/ | 3.4 | 0.3 | 9% | 3 |
| R403E/E410N | R233E/E240N | | | | |
| R318Y/R338E/T343R/ | R150Y/R170E/T175R/ | 4.6 | 1.2 | 26% | 5 |
| N346Y/R403E/E410N | N178Y/R233E/E240N | | | | |
| T343R/N346D | T175R/N178D | 1.9 | 0.4 | 21% | 2 |
| R318Y/R338E/T343R/ | R150Y/R170E/T175R/ | 5.4 | 2.0 | 36% | 2 |
| N346D/R403E/E410N | N178D/R233E/E240N | | | | |
| R318Y/R338E/Y345A/ | R150Y/R170E/Y177A/ | 1.4 | 0.5 | 36% | 6 |
| R403E/E410N | R233E/E240N | | | | |
| R318Y/R338E/Y345A/ | R150Y/R170E/Y177A/ | 1.2 | 0.3 | 26% | 3 |
| N346D/R403E/E410N | N178D/R233E/E240N | | | | |
| Y155F/K247N/N249S/ | Y[155]F/K82N/N84S/ | 5.7 | 3.2 | 55% | 5 |
| R318Y/R338E/R403E | R150Y/R170E/R233E | | | | |
| K247N/N249S/R318Y/ | K82N/N84S/R150Y/ | 10.5 | 3.6 | 34% | 2 |
| R338E/R403E | R170E/R233E | | | | |
| Y155F/K247N/N249S/ | Y[155]F/K82N/N84S/ | 1.2 | 0.5 | 40% | 3 |
| R318Y/R403E/E410N | R150Y/R233E/E240N | | | | |
| K247N/N249S/R318Y/ | K82N/N84S/R150Y/ | 2.9 | 1.6 | 55% | 10 |
| R403E/E410N | R233E/E240N | | | | |
| Y155F/K247N/N249S/ | Y[155]F/K82N/N84S/ | 5.0 | 0.6 | 13% | 3 |
| R338E/R403E/E410N | R170E/R233E/E240N | | | | |
| K247N/N249S/R338E/ | K82N/N84S/R170E/ | 4.8 | 0.8 | 17% | 2 |
| R403E/E410N | R233E/E240N | | | | |
| R318Y/R338E/T343R/ | R150Y/R170E/T175R/ | 6.7 | 1.6 | 24% | 4 |
| R403E | R233E | | | | |
| Y155F/R318Y/R338E/ | Y[155]F/R150Y/R170E/ | 8.2 | 4.1 | 50% | 4 |
| T343R/R403E | T175R/R233E | | | | |
| R318Y/R338E/T343R/ | R150Y/R170E/T175R/ | 4.9 | 1.2 | 24% | 16 |
| E410N | E240N | | | | |
| Y155F/R318Y/R338E/ | Y[155]F/R150Y/R170E/ | 9.2 | 3.1 | 33% | 4 |
| T343R/E410N | T175R/E240N | | | | |
| R318Y/T343R/R403E/ | R150Y/T175R/R233E/ | 5.3 | 0.9 | 17% | 3 |
| E410N | E240N | | | | |
| Y155F/R318Y/T343R/ | Y[155]F/R150Y/T175R/ | 8.8 | 0.3 | 4% | 2 |
| R403E/E410N | R233E/E240N | | | | |
| R338E/T343R/R403E/ | R170E/T175R/R233E/ | 9.8 | 1.4 | 15% | 2 |
| E410N | E240N | | | | |
| Y155F/R338E/T343R/ | Y[155]F/R170E/T175R/ | 5.7 | 1.1 | 20% | 4 |
| R403E/E410N | R233E/E240N | | | | |
| Y155F/K247N/N249S/ | Y[155]F/K82N/N84S/ | 9.7 | 3.4 | 35% | 11 |
| R318Y/R338E/T343R/ | R150Y/R170E/T175R/ | | | | |
| R403E/E410N | R233E/E240N | | | | |
| K247N/N249S/R318Y/ | K82N/N84S/R150Y/ | 10.6 | 3.6 | 34% | 5 |
| R338E/T343R/R403E/ | R170E/T175R/R233E/ | | | | |
| E410N | E240N | | | | |
| K228N/I251S/R318Y/ | K63N/I86S/R150Y/ | 7.5 | 3.3 | 44% | 7 |
| R338E/R403E/E410N | R170E/R233E/E240N | | | | |
| Y155F/K228N/I251S/ | Y[155]F/K63N/I86S/ | 5.3 | 1.9 | 36% | 5 |
| R318Y/R338E/R403E/ | R150Y/R170E/R233E/ | | | | |
| E410N | E240N | | | | |
| N260S/R318Y/R338E/ | N95S/R150Y/R170E/ | 8.9 | 3.6 | 40% | 7 |
| T343R/R403E/E410N | T175R/R233E/E240N | | | | |
| Y155F/N260S/R318Y/ | Y[155]F/N95S/R150Y/ | 5.8 | 1.6 | 28% | 5 |
| R338E/T343R/R403E/ | R170E/T175R/R233E/ | | | | |
| E410N | E240N | | | | |
| K228N/K247N/N249S/ | K63N/K82N/N84S/ | 9.9 | 3.2 | 32% | 12 |
| R318Y/R338E/T343R/ | R150Y/R170E/T175R/ | | | | |
| R403E/E410N | R233E/E240N | | | | |
| Y155F/K228N/K247N/ | Y[155]F/K63N/K82N/ | 9.4 | 2.3 | 25% | 5 |
| N249S/R318Y/R338E/ | N84S/R150Y/R170E/ | | | | |
| T343R/R403E/E410N | T175R/R233E/E240N | | | | |
| Y155F/R338E/T343R/ | Y[155]F/R170E/T175R/ | 5.2 | 0.9 | 18% | 5 |
| R403E | R233E | | | | |
| R338E/T343R/R403E | R170E/T175R/R233E | 6.9 | 0.3 | 4% | 2 |
| Y155F/R338E/T343R/ | Y[155]F/R170E/T175R/ | 6.8 | 2.4 | 34% | 6 |
| R403E/E410S | R233E/E240S | | | | |
| Y155F/N260S/R338E/ | Y[155]F/N95S/R170E/ | 6.4 | 3.8 | 59% | 6 |
| T343R/R403E | T175R/R233E | | | | |
| Y155F/I251S/R338E/ | Y[155]F/I86S/R170E/ | 5.9 | 0.7 | 12% | 2 |
| T343R/R403E | T175R/R233E | | | | |
| R318Y/R338E/T343R/ | R150Y/R170E/T175R/ | 7.6 | 1.7 | 22% | 14 |
| R403E/E410S | R233E/E240S | | | | |
| Y155F/K247N/N249S/ | Y[155]F/K82N/N84S/ | 4.7 | 0.2 | 5% | 4 |
| T343R/R403E | T175R/R233E | | | | |
| Y155F/K247N/N249S/ | Y[155]F/K82N/N84S/ | 10.6 | 0.8 | 8% | 2 |
| R318Y/R338E/T343R/ | R150Y/R170E/T175R/ | | | | |
| R403E | R233E | | | | |
| K247N/N249S/R318Y/ | K82N/N84S/R150Y/ | 9.2 | 3.3 | 36% | 4 |
| R338E/T343R/R403E | R170E/ | | | | |
| | T175R/R233E | | | | |
| Y155F/K247N/N249S/ | Y[155]F/K82N/N84S/ | 9.8 | 0.7 | 8% | 2 |
| R338E/T343R/R403E/ | R170E/T175R/R233E/ | | | | |
| E410N | E240N | | | | |
| K247N/N249S/R338E/ | K82N/N84S/R170E/ | 10.8 | 1.6 | 15% | 2 |
| T343R/R403E/E410N | T175R/R233E/E240N | | | | |
| Y155F/K247N/N249S/ | Y[155]F/K82N/N84S/ | 7.5 | 1.5 | 20% | 2 |
| R318Y/R338E | R150Y/R170E | | | | |
| Y155F/K247N/N249S/ | Y[155]F/K82N/N84S/ | 10.3 | 3.3 | 32% | 4 |
| R318Y/T343R | R150Y/T175R | | | | |
| Y155F/K247N/N249S/ | Y[155]F/K82N/N84S/ | 1.7 | 0.7 | 42% | 3 |
| R318Y/R403E | R150Y/R233E | | | | |
| Y155F/K247N/N249S/ | Y[155]F/K82N/N84S/ | 3.4 | 0.9 | 26% | 3 |
| R318Y/E410N | R150Y/E240N | | | | |
| Y155F/K247N/N249S/ | Y[155]F/K82N/N84S/ | 5.3 | 0.6 | 11% | 2 |
| R338E/R403E | R170E/R233E | | | | |
| Y155F/K247N/N249S/ | Y[155]F/K82N/N84S/ | 10.6 | 1.1 | 10% | 2 |
| R338E/T343R | R170E/T175R | | | | |
| Y155F/K247N/N249S/ | Y[155]F/K82N/N84S/ | 7.7 | 2.3 | 30% | 4 |
| R318Y/R338E/T343R/ | R150Y/R170E/T175R/ | | | | |
| E410N | E240N | | | | |
| K247N/N249S/R318Y/ | K82N/N84S/R150Y/ | 8.8 | 4.4 | 50% | 6 |
| R338E/T343R/E410N | R170E/T175R/E240N | | | | |
| Y155F/K247N/N249S/ | Y[155]F/K82N/N84S/ | 9.0 | 0.4 | 5% | 2 |
| R318Y/T343R/R403E/ | R150Y/T175R/R233E/ | | | | |
| E410N | E240N | | | | |
| K247N/N249S/R318Y/ | K82N/N84S/R150Y/ | 9.5 | 1.6 | 17% | 7 |
| T343R/R403E/E410N | T175R/R233E/E240N | | | | |
| Y155F/K247N/N249S/ | Y[155]F/K82N/N84S/ | 7.3 | 3.5 | 48% | 8 |
| R338E/E410N | R170E/E240N | | | | |
| Y155F/K247N/N249S/ | Y[155]F/K82N/N84S/ | 7.5 | 2.1 | 28% | 2 |
| R318Y/T343R/R403E | R150Y/T175R/R233E | | | | |
| K247N/N249S/R318Y/ | K82N/N84S/R150Y/ | 3.7 | 1.6 | 44% | 9 |
| T343R/R403E | T175R/R233E | | | | |
| Y155F/K247N/N249S/ | Y[155]F/K82N/N84S/ | 8.1 | 4.1 | 51% | 4 |
| R318Y/T343R/E410N | R150Y/T175R/E240N | | | | |
| K247N/N249S/R318Y/ | K82N/N84S/R150Y/ | 6.1 | 2.6 | 42% | 4 |
| T343R/E410N | T175R/E240N | | | | |
| Y155F/K247N/N249S/ | Y[155]F/K82N/N84S/ | 14.6 | 0.2 | 1% | 2 |
| R338E/T343R/R403E | R170E/T175R/R233E | | | | |
| K247N/N249S/R338E/ | K82N/N84S/R170E/ | 14.6 | 0.4 | 3% | 2 |
| T343R/R403E | T175R/R233E | | | | |
| Y155F/K247N/N249S/ | Y[155]F/K82N/N84S/ | 4.8 | 1.0 | 20% | 2 |
| R338E/T343R/E410N | R170E/T175R/E240N | | | | |
| K247N/N249S/R338E/ | K82N/N84S/R170E/ | 7.9 | 1.4 | 18% | 5 |
| T343R/E410N | T175R/E240N | | | | |
| Y155F/K247N/N249S/ | Y[155]F/K82N/N84S/ | 15.0 | 3.0 | 20% | 2 |
| T343R/R403E/E410N | T175R/R233E/E240N | | | | |
| K247N/N249S/T343R/ | K82N/N84S/T175R/ | 8.0 | 2.8 | 36% | 2 |
| R403E/E410N | R233E/E240N | | | | |
| Y155F/R318Y/R338E/ | Y[155]F/R150Y/R170E/ | 7.9 | 3.0 | 38% | 7 |
| T343R | T175R | | | | |
| R318Y/R338E/T343R | R150Y/R170E/T175R | 4.5 | 1.2 | 27% | 2 |
| Y155F/R318Y/T343R/ | Y[155]F/R150Y/T175R/ | 5.0 | 1.1 | 22% | 2 |
| R403E | R233E | | | | |
| Y155F/T343R/R403E/ | Y[155]F/T175R/R233E/ | 6.6 | 1.4 | 21% | 2 |
| E410N | E240N | | | | |
| Y155F/K247N/N249S/ | Y[155]F/K82N/N84S/ | 8.5 | 3.3 | 39% | 7 |
| R318Y/R338E/T343R | R150Y/R170E/T175R | | | | |
| K247N/N249S/R318Y/ | K82N/N84S/R150Y/ | 8.0 | 1.7 | 22% | 4 |
| R338E/T343R | R170E/T175R | | | | |
| Y155F/K247N/N249S/ | Y[155]F/K82N/N84S/ | 7.9 | 1.6 | 20% | 5 |
| T343R/E410N | T175R/E240N | | | | |
| Y155F/K247N/N249S/ | Y[155]F/K82N/N84S/ | 2.7 | 1.4 | 52% | 7 |
| R403E/E410N | R233E/E240N | | | | |
| Y155F/R338E/T343R/ | Y[155]F/R170E/T175R/ | 6.0 | 2.2 | 37% | 6 |
| E410N | E240N | | | | |
| R338E/T343R/E410N | R170E/T175R/E240N | 3.1 | 0.5 | 16% | 2 |
| Y155F/R318Y/T343R/ | Y[155]F/R150Y/T175R/ | 5.0 | 1.3 | 25% | 4 |
| E410N | E240N | | | | |
| R318Y/T343R/E410N | R150Y/T175R/E240N | 3.2 | 0.4 | 13% | 2 |
| K228N/R318Y/R338E/ | K63N/R150Y/R170E/ | 10.5 | 0.7 | 6% | 3 |
| T343R/R403E/E410N | T175R/R233E/E240N | | | | |
| K228N/K247N/N249S/ | K63N/K82N/N84S/ | 10.9 | 1.4 | 13% | 3 |
| R318Y/R338E/T343R/ | R150Y/R170E/T175R/ | | | | |
| R403E | R233E | | | | |
| K228N/247N/N249S/ | K63N/K82N/N84S/ | 4.7 | 0.4 | 8% | 2 |
| R318Y/R338E/T343R/ | R150Y/R170E/T175R/ | | | | |
| E410N | E240N | | | | |
| K228N/K247N/N249S/ | K63N/K82N/N84S/ | 8.1 | 2.1 | 26% | 3 |
| R318Y/T343R/R403E/ | R150Y/T175R/R233E/ | | | | |
| E410N | E240N | | | | |

†produced in BHK-21 cells;
*80% glycosylated form of E410N

TABLE 27
Catalytic activity of FIXa variants (K_{M})

| Mutation | Mutation | | | | |
|---|---|---|---|---|---|
| (Mature FIX | (Chymotrypsin | K_{M} | ±S.D. | | |
| Numbering) | Numbering) | (nM) | (nM) | % CV | n |
| BeneFIX ® | BeneFIX ® | 76.9 | 27.5 | 36% | 125 |
| Coagulation FIX | Coagulation FIX | | | | |
| (T148A) | (T[148]A) | | | | |
| Plasma Purified | Plasma Purified | 74.5 | 25.5 | 34% | 120 |
| FIXa | FIXa | | | | |
| Catalyst | Catalyst | 74.7 | 23.1 | 31% | 31 |
| Biosciences WT | Biosciences WT | | | | |
| N157D | N[157]D | 121.8 | 53.0 | 44% | 2 |
| Y155F | Y[155]F | 90.3 | 10.3 | 11% | 2 |
| A103N/N105S/Y155F | A[103]N/N[105]S/ | 80.4 | 2.5 | 3% | 2 |
| | Y[155]F | | | | |
| D104N/K106S/Y155F | D[104]N/K[106]S/ | 81.5 | 5.2 | 6% | 2 |
| | Y[155]F | | | | |
| A103N/N105S | A[103]N/N[105]S | 88.0 | 22.5 | 26% | 9 |
| D104N/K106S | D[104]N/K[106]S | 83.2 | 18.2 | 22% | 9 |
| K106N/V108S | K[106]N/V[108]S | 91.9 | 20.2 | 22% | 7 |
| D85N | D[85]N | 64.5 | 23.1 | 36% | 15 |
| T148A | T[148]A | 64.5 | 25.1 | 39% | 30 |
| T148A† | T[148]A† | 74.6 | 16.1 | 22% | 7 |
| K5A | K[5]A | 55.0 | 0.3 | 1% | 2 |
| D64N | D[64]N | 121.4 | 58.8 | 48% | 2 |
| D64A | D[64]A | 129.4 | 36.3 | 28% | 2 |
| N167D | N[167]D | 94.6 | 7.0 | 7% | 2 |
| N167Q | N[167]Q | 77.1 | 35.8 | 46% | 4 |
| S61A | S[61]A | 84.6 | 35.6 | 42% | 4 |
| S53A | S[53]A | 109.9 | 11.6 | 11% | 3 |
| T159A | T[159]A | 100.9 | 1.2 | 1% | 3 |
| T169A | T[169]A | 99.7 | 10.8 | 11% | 3 |
| T172A | T[172]A | 96.2 | 22.1 | 23% | 3 |
| T179A | T[179]A | 94.5 | 16.7 | 18% | 3 |
| Y155H | Y[155]H | 93.9 | 15.8 | 17% | 3 |
| Y155Q | Y[155]Q | 87.6 | 29.8 | 34% | 3 |
| S158A | S[158]A | 107.7 | 0.4 | 0% | 2 |
| S158D | S[158]D | 87.0 | 9.0 | 10% | 2 |
| S158E | S[158]E | 96.0 | 14.1 | 15% | 2 |
| N157Q | N[157]Q | 107.8 | 5.5 | 5% | 2 |
| D203N/F205T | D39N/F41T | 74.3 | 19.5 | 26% | 12 |
| D85N/D203N/F205T | D[85]N/D39N/F41T | 40.6 | 9.1 | 22% | 5 |
| K228N | K63N | 72.5 | 25.5 | 35% | 13 |
| D85N/K228N | D[85]N/K63N | 60.1 | 13.4 | 22% | 6 |
| A103N/N105S/K228N | A[103]N/N[105]S/ | 76.5 | 15.8 | 21% | 3 |
| | K63N | | | | |
| D104N/K106S/K228N | D[104]N/K[106]S/ | 96.8 | 21.2 | 22% | 3 |
| | K63N | | | | |
| Y155F/K228N | Y[155]F/K63N | 73.7 | 3.7 | 5% | 2 |
| D104N/K106S/Y155F/ | D[104]N/K[106]S/ | 76.2 | 6.4 | 8% | 2 |
| K228N | Y[155]F/K63N | | | | |
| I251S | I86S | 64.3 | 13.3 | 21% | 13 |
| D85N/I251S | D[85]N/I86S | 51.5 | 15.3 | 30% | 5 |
| D85N/D104N/K106S/ | D[85]N/D[104]N/ | 46.4 | 19.0 | 41% | 5 |
| I251S | K[106]S/I86S | | | | |
| A103N/N105S/I251S | A[103]N/N[105]S/ | 90.9 | 41.2 | 45% | 3 |
| | I86S | | | | |
| D104N/K106S/I251S | D[104]N/K[106]S/ | 97.5 | 13.8 | 14% | 2 |
| | I86S | | | | |
| Y155F/I251S | Y[155]F/I86S | 56.4 | 17.5 | 31% | 2 |
| A262S | A95bS | 99.2 | 19.9 | 20% | 8 |
| K413N | K243N | 109.6 | 41.0 | 37% | 5 |
| E410N | E240N | 46.2 | 21.5 | 47% | 21 |
| E410N* | E240N* | 83.3 | 36.9 | 44% | 11 |
| E239N | E74N | 78.3 | 29.5 | 38% | 9 |
| T241N/H243S | T76N/H78S | 104.5 | 3.5 | 3% | 2 |
| K247N/N249S | K82N/N84S | 75.0 | 15.4 | 21% | 11 |
| Y155F/K247N/N249S | Y[155]F/K82N/N84S | 67.1 | 23.6 | 35% | 4 |
| A103N/N105S/K247N/ | A[103]N/N[105]S/ | 84.0 | 9.7 | 12% | 6 |
| N249S | K82N/N84S | | | | |
| D104N/K106S/K247N/ | D[104]N/K[106]S/ | 102.3 | 23.0 | 23% | 2 |
| N249S | K82N/N84S | | | | |
| D104N/K106S/Y155F/ | D[104]N/K[106]S/ | 89.3 | 10.3 | 12% | 3 |
| K247N/N249S | Y[155]F/K82N/N84S | | | | |
| L321N | L153N | 118.5 | 10.6 | 9% | 2 |
| F314N/H315S | F145N/H147S | No | n.d. | n.d. | 4 |
| | | Activity | | | |
| S319N/L321S | S151N/L153S | 54.2 | 14.8 | 27% | 3 |
| N260S | N95S | 83.4 | 27.5 | 33% | 13 |
| D104N/K106S/N260S | D[104]N/K[106]S/ | 94.3 | 6.8 | 7% | 2 |
| | N95S | | | | |
| Y155F/N260S | Y[155]F/N95S | 130.6 | 78.1 | 60% | 2 |
| D104N/K106S/Y155F/ | D[104]N/K[106]S/ | 107.7 | 74.8 | 69% | 2 |
| N260S | Y[155]F/N95S | | | | |
| Y284N | Y117N | 59.8 | 23.5 | 39% | 8 |
| G317N | G149N | No | n.d. | n.d. | 5 |
| | | Activity | | | |
| R318N/A320S | R150N/A152S | No | n.d. | n.d. | 8 |
| | | Activity | | | |
| R318A | R150A | 52.8 | 25.8 | 49% | 3 |
| R318E | R150E | 33.6 | 10.3 | 31% | 3 |
| R318Y | R150Y | 40.7 | 7.6 | 19% | 3 |
| R312Q | R143Q | 29.9 | 5.0 | 17% | 3 |
| R312A | R143A | 61.6 | 16.9 | 27% | 2 |
| R312Y | R143Y | 27.2 | 11.4 | 42% | 2 |
| R312L | R143L | 28.8 | 0.6 | 2% | 2 |
| V202M | V38M | 40.2 | 1.0 | 2% | 2 |
| V202Y | V38Y | 70.6 | 2.3 | 3% | 2 |
| D203M | D39M | 40.6 | 7.9 | 19% | 5 |
| D203Y | D39Y | 58.0 | 19.5 | 34% | 4 |
| A204M | A40M | 34.0 | 9.2 | 27% | 5 |
| A204Y | A40Y | 39.5 | 10.3 | 26% | 2 |
| K400A/R403A | K230A/R233A | 56.7 | 10.0 | 18% | 2 |
| K400E/R403E | K230E/R233E | No | n.d. | n.d. | 4 |
| | | Activity | | | |
| R403A | R233A | 46.4 | 5.2 | 11% | 7 |
| R403E | R233E | 67.0 | 19.4 | 29% | 6 |
| K400A | K230A | 74.6 | 22.1 | 30% | 2 |
| K400E | K230E | 61.3 | 9.3 | 15% | 2 |
| K293E | K126E | 63.2 | 13.9 | 22% | 2 |
| K293A | K126A | 73.7 | 35.2 | 48% | 2 |
| R333A | R165A | No | n.d. | n.d. | 2 |
| | | Activity | | | |
| R333E | R165E | No | n.d. | n.d. | 2 |
| | | Activity | | | |
| R338A | R170A | 33.7 | 3.7 | 11% | 2 |
| R338E | R170E | 28.7 | 9.0 | 31% | 10 |
| R338A/R403A | R170A/R233A | 73.6 | 18.1 | 25% | 6 |
| R338E/R403E | R170E/R233E | 51.9 | 11.9 | 23% | 2 |
| K293A/R403A | K126A/R233A | 69.2 | 10.2 | 15% | 2 |
| K293E/R403E | K126E/R233E | 104.1 | 31.0 | 30% | 2 |
| K293A/R338A/R403A | K126A/R170A/R233A | 65.4 | 1.3 | 2% | 2 |
| K293E/R338E/R403E | K126E/R170E/R233E | 50.0 | 15.1 | 30% | 2 |
| R318A/R403A | R150A/R233A | 45.7 | 1.6 | 3% | 2 |
| R318E/R403E | R150E/R233E | 75.3 | 47.7 | 63% | 2 |
| R318Y/E410N | R150Y/E240N | 49.6 | 14.3 | 29% | 21 |
| R338E/E410N | R170E/E240N | 12.6 | 4.2 | 33% | 8 |
| R338E/R403E/E410N | R170E/R233E/E240N | 45.5 | 12.8 | 28% | 7 |
| R318Y/R338E/R403E | R150Y/R170E/R233E | 53.7 | 1.9 | 4% | 2 |
| D203N/F205T/K228N | D39N/F41T/K63N | 39.9 | 3.8 | 9% | 2 |
| D203N/F205T/E410N | D39N/F41T/E240N | 45.5 | 12.0 | 26% | 6 |
| D203N/F205T/R338E | D39N/F41T/R170E | 24.1 | 5.6 | 23% | 2 |
| D203N/F205T/R338A | D39N/F41T/R170A | 38.5 | 9.9 | 26% | 3 |
| D203N/F205T/R318Y | D39N/F41T/R150Y | 47.5 | 6.4 | 13% | 4 |
| D203N/F205T/R338E/ | D39N/F41T/R170E/ | 51.1 | 10.7 | 21% | 2 |
| R403E | R233E | | | | |
| K228N/E410N | K63N/E240N | 44.3 | 13.0 | 29% | 10 |
| K228N/R338E | K63N/R170E | 23.1 | 3.0 | 13% | 2 |
| K228N/R338A | K63N/R170A | 31.2 | 4.5 | 14% | 2 |
| K228N/R318Y | K63N/R150Y | 61.3 | 5.4 | 9% | 5 |
| K228N/R338E/R403E | K63N/R170E/R233E | 59.2 | 4.9 | 8% | 2 |
| R403E/E410N | R233E/E240N | 93.7 | 1.0 | 1% | 2 |
| R318Y/R338E/E410N | R150Y/R170E/E240N | 14.2 | 4.3 | 30% | 26 |
| D104N/K106S/R318Y/ | D[104]N/K[106]S/ | 18.9 | 4.1 | 22% | 4 |
| R338E/E410N | R150Y/R170E/E240N | | | | |
| Y155F/R318Y/R338E/ | Y[155]F/R150Y/R170E/ | 16.0 | 4.8 | 30% | 5 |
| E410N | E240N | | | | |
| K228N/R318Y/E410N | K63N/R150Y/E240N | 42.0 | 4.7 | 11% | 4 |
| R318Y/R403E/E410N | R150Y/R233E/E240N | 88.3 | 12.4 | 14% | 3 |
| R318Y/R338E/R403E/ | R150Y/R170E/R233E/ | 45.5 | 12.2 | 27% | 14 |
| E410N | E240N | | | | |
| A103N/N105S/R318Y/ | A[103]N/N[105]S/ | 44.7 | 20.9 | 47% | 5 |
| R338E/R403E/E410N | R150Y/R170E/R233E/ | | | | |
| | E240N | | | | |
| D104N/K106S/R318Y/ | D[104]N/K[106]S/ | 38.5 | 16.1 | 42% | 3 |
| R338E/R403E/E410N | R150Y/R170E/R233E/ | | | | |
| | E240N | | | | |
| Y155F/R318Y/R338E/ | Y[155]F/R150Y/R170E/ | 30.4 | 10.5 | 35% | 4 |
| R403E/E410N | R233E/E240N | | | | |
| A103N/N105S/Y155F/ | A[103]N/N[105]S/ | 50.7 | 4.5 | 9% | 2 |
| R318Y/R338E/R403E/ | Y[155]F/R150Y/R170E/ | | | | |
| E410N | R233E/E240N | | | | |
| D104N/K106S/Y155F/ | D[104]N/K[106]S/ | 48.0 | 2.1 | 4% | 2 |
| R318Y/R338E/R403E/ | Y[155]F/R150Y/R170E/ | | | | |
| E410N | R233E/E240N | | | | |
| D203N/F205T/R318Y/ | D39N/F41T/R150Y/ | 45.7 | 13.4 | 29% | 6 |
| E410N | E240N | | | | |
| R333S | R165S | 605.9 | 317.5 | 52% | 3 |
| R338L | R170L | 47.9 | 9.0 | 19% | 3 |
| K316N | K148N | 62.5 | 15.6 | 25% | 3 |
| K316A | K148A | 55.2 | 4.1 | 7% | 3 |
| K316E | K148E | 110.5 | 25.1 | 23% | 3 |
| K316S | K148S | 57.3 | 4.6 | 8% | 3 |
| K316M | K148M | 26.0 | 16.7 | 64% | 3 |
| E239S | E74S | 28.5 | 19.2 | 67% | 3 |
| E239A | E74A | 55.4 | 18.4 | 33% | 3 |
| E239R | E74R | 58.3 | 13.9 | 24% | 3 |
| E239K | E74K | 59.2 | 25.5 | 43% | 3 |
| H257F | H92F | 62.0 | 30.1 | 49% | 3 |
| H257Y | H92Y | 59.3 | 25.0 | 42% | 3 |
| H257E | H92E | 59.7 | 39.6 | 66% | 3 |
| H257S | H92S | 56.0 | 24.7 | 44% | 3 |
| T412A | T242A | 76.1 | 44.7 | 59% | 5 |
| T412V | T242V | 51.2 | 18.9 | 37% | 8 |
| E410N/T412A | E240N/T242A | 37.2 | 3.6 | 10% | 4 |
| E410N/T412V | E240N/T242V | 33.3 | 4.9 | 15% | 4 |
| E410Q | E240Q | 56.1 | 18.0 | 32% | 4 |
| E410S | E240S | 50.0 | 11.9 | 24% | 12 |
| E410A | E240A | 47.7 | 11.7 | 24% | 10 |
| E410D | E240D | 71.9 | 26.9 | 37% | 4 |
| N346D | N178D | 45.7 | 7.8 | 17% | 4 |
| Y155F/N346D | Y[155]F/N178D | 104.4 | 14.5 | 14% | 2 |
| N346Y | N178Y | 27.4 | 4.2 | 15% | 8 |
| Y345A | Y177A | 50.8 | 32.4 | 64% | 4 |
| Y345T | Y177T | 28.6 | 7.9 | 28% | 4 |
| T343R | T175R | 31.3 | 10.9 | 35% | 9 |
| T343E | T175E | 27.3 | 10.0 | 37% | 4 |
| T343Q | T175Q | 37.0 | 9.1 | 25% | 3 |
| F342I | F174I | 30.0 | 19.1 | 64% | 3 |
| T343R/Y345T | T175R/Y177T | 26.5 | 6.8 | 26% | 3 |
| R318Y/R338E | R150Y/R170E | 24.6 | 5.5 | 22% | 4 |
| Y259F/K265T/Y345T | Y94F/K98T/Y177T | 30.9 | 4.8 | 16% | 2 |
| K228N/I251S | K63N/I86S | 122.6 | 53.5 | 44% | 2 |
| K228N/R318Y/R338E/ | K63N/R150Y/R170E/ | 36.1 | 14.0 | 39% | 3 |
| R403E/E410N | R233E/E240N | | | | |
| Y155F/K228N/R318Y/ | Y[155]F/K63N/R150Y/ | 48.0 | 9.8 | 21% | 2 |
| R338E/R403E/E410N | R170E/R233E/E240N | | | | |
| D85N/K228N/R318Y/ | D[85]N/K63N/R150Y/ | 39.3 | 9.8 | 25% | 2 |
| R338E/R403E/E410N | R170E/R233E/E240N | | | | |
| I251S/R318Y/R338E/ | I86S/R150Y/R170E/ | 33.4 | 10.2 | 30% | 4 |
| R403E/E410N | R233E/E240N | | | | |
| D104N/K106S/I251S/ | D[104]N/K[106]S/ | 46.2 | 7.7 | 17% | 8 |
| R318Y/R338E/R403E/ | I86S/R150Y/R170E/ | | | | |
| E410N | R233E/E240N | | | | |
| Y155F/I251S/R318Y/ | Y[155]F/I86S/R150Y/ | 43.3 | 7.0 | 16% | 2 |
| R338E/R403E/E410N | R170E/R233E/E240N | | | | |
| I251S/R318Y/R338E/ | I86S/R150Y/R170E/ | 16.2 | 1.8 | 11% | 7 |
| E410N | E240N | | | | |
| D104N/K106S/I251S/ | D[104]N/K[106]S/ | 24.3 | 8.6 | 35% | 3 |
| R318Y/R338E/E410N | I86S/R150Y/R170E/ | | | | |
| | E240N | | | | |
| F314N/K316S | F145N/K148S | 635.1 | 569.9 | 90% | 2 |
| K247N/N249S/R318Y/ | K82N/N84S/R150Y/ | 39.2 | 8.3 | 21% | 6 |
| R338E/R403E/E410N | R170E/R233E/E240N | | | | |
| Y155F/K247N/N249S/ | Y[155]F/K82N/N84S/ | 39.1 | 14.7 | 38% | 6 |
| R318Y/R338E/R403E/ | R150Y/R170E/R233E/ | | | | |
| E410N | E240N | | | | |
| A103N/N105S/K247N/ | A[103]N/N[105]S/ | 39.7 | 4.5 | 11% | 2 |
| N249S/R318Y/R338E/ | K82N/N84S/R150Y/ | | | | |
| R403E/E410N | R170E/R233E/E240N | | | | |
| D104N/K106S/K247N/ | D[104]N/K[106]S/ | 59.0 | 0.6 | 1% | 2 |
| N249S/R318Y/R338E/ | K82N/N84S/R150Y/ | | | | |
| R403E/E410N | R170E/R233E/E240N | | | | |
| K247N/N249S/R318Y/ | K82N/N84S/R150Y/ | 16.6 | 3.7 | 22% | 6 |
| R338E/E410N | R170E/E240N | | | | |
| Y155F/K247N/N249S/ | Y[155]F/K82N/N84S/ | 15.3 | 4.1 | 27% | 7 |
| R318Y/R338E/E410N | R150Y/R170E/E240N | | | | |
| R318Y/R338E/R403E/ | R150Y/R170E/R233E/ | 35.1 | 12.4 | 35% | 4 |
| E410S | E240S | | | | |
| R318Y/R338E/E410S | R150Y/R170E/E240S | 16.4 | 4.0 | 25% | 8 |
| K228N/K247N/N249S | K63N/K82N/N84S | 94.5 | 27.0 | 29% | 2 |
| D104N/K106S/Y155F/ | D[104]N/K[106]S/ | 75.3 | 26.4 | 35% | 2 |
| K228N/K247N/N249S | Y[155]F/K63N/K82N/ | | | | |
| | N84S | | | | |
| D104N/K106S/K228N/ | D[104]N/K[106]S/ | 77.1 | 18.3 | 24% | 5 |
| K247N/N249S | K63N/K82N/N84S | | | | |
| Y155F/K228N/K247N/ | Y[155]F/K63N/K82N/ | 79.2 | 27.6 | 35% | 2 |
| N249S | N84S | | | | |
| K228N/K247N/N249S/ | K63N/K82N/N84S/ | 55.8 | 15.8 | 28% | 3 |
| R318Y/R338E/R403E/ | R150Y/R170E/R233E/ | | | | |
| E410N | E240N | | | | |
| R318Y/R338E/R403E/ | R150Y/R170E/R233E/ | 44.3 | 19.2 | 43% | 4 |
| E410N/T412V | E240N/T242V | | | | |
| R318Y/R338E/R403E/ | R150Y/R170E/R233E/ | 33.5 | 4.8 | 14% | 4 |
| E410N/T412A | E240N/T242A | | | | |
| R318Y/R338E/R403E/ | R150Y/R170E/R233E/ | 67.5 | 11.6 | 17% | 4 |
| T412A | T242A | | | | |
| R318Y/R338E/T412A | R150Y/R170E/T242A | 23.5 | 5.3 | 22% | 6 |
| R318Y/R338E/E410N/ | R150Y/R170E/E240N/ | 29.7 | 10.9 | 37% | 4 |
| T412V | T242V | | | | |
| N260S/R318Y/R338E/ | N95S/R150Y/R170E/ | 72.4 | 20.2 | 28% | 2 |
| R403E/E410N | R233E/E240N | | | | |
| D104N/K106S/N260S/ | D[104]N/K[106]S/ | 61.1 | 0.0 | 0% | 2 |
| R318Y/R338E/R403E/ | N95S/R150Y/R170E/ | | | | |
| E410N | R233E/E240N | | | | |
| Y155F/N260S/R318Y/ | Y[155]F/N95S/R150Y/ | 83.9 | 4.4 | 5% | 2 |
| R338E/R403E/E410N | R170E/R233E/E240N | | | | |
| R318Y/R338E/N346D/ | R150Y/R170E/N178D/ | 77.7 | 20.9 | 27% | 2 |
| R403E/E410N | R233E/E240N | | | | |
| Y155F/R318Y/R338E/ | Y[155]F/R150Y/R170E/ | 100.0 | 15.6 | 16% | 2 |
| N346D/R403E/E410N | N178D/R233E/E240N | | | | |
| K247N/N249S/N260S | K82N/N84S/N95S | 114.1 | 0.0 | 0% | 2 |
| Y155F/K247N/N249S/ | Y[155]F/K82N/N84S/ | 96.5 | 5.5 | 6% | 2 |
| N260S | N95S | | | | |
| D104N/K106S/K247N/ | D[104]N/K[106]S/ | 61.2 | 14.1 | 23% | 2 |
| N249S/N260S | K82N/N84S/N95S | | | | |
| D104N/K106S/Y155F/ | D[104]N/K[106]S/ | 68.5 | 33.2 | 49% | 2 |
| K247N/N249S/N260S | Y[155]F/K82N/N84S/ | | | | |
| | N95S | | | | |
| K247N/N249S/N260S/ | K82N/N84S/N95S/ | 62.2 | 0.0 | 0% | 2 |
| R318Y/R338E/R403E/ | R150Y/R170E/R233E/ | | | | |
| E410N | E240N | | | | |
| Y155F/N260S/N346D | Y[155]F/N95S/N178D | 127.9 | 6.2 | 5% | 2 |
| R318Y/R338E/T343R/ | R150Y/R170E/T175R/ | 22.3 | 5.0 | 23% | 3 |
| R403E/E410N | R233E/E240N | | | | |
| R338E/T343R | R170E/T175R | 13.6 | 3.7 | 27% | 4 |

†produced in BHK-21 cells;
*80% glycosylated form of E410N

TABLE 28
Catalytic activity of FIXa variants (K_{M})

| Mutation | Mutation | | | | |
|---|---|---|---|---|---|
| (Mature FIX | (Chymotrypsin | K_{M} | ±S.D. | | |
| Numbering) | Numbering) | (nM) | (nM) | % CV | n |
| BeneFIX ® Coagulation | BeneFIX ® Coagulation | 75.8 | 27.2 | 36% | 140 |
| FIX (T148A) | FIX (T[148]A) | | | | |
| Plasma Purified FIXa | Plasma Purified FIXa | 73.3 | 26.8 | 37% | 200 |
| Catalyst Biosciences WT | Catalyst Biosciences WT | 72.3 | 24.3 | 34% | 33 |
| N157D | N[157]D | 121.8 | 53.0 | 44% | 2 |
| Y155F | Y[155]F | 90.3 | 10.3 | 11% | 2 |
| A103N/N105S/Y155F | A[103]N/N[105]S/Y[155]F | 80.4 | 2.5 | 3% | 2 |
| D104N/K106S/Y155F | D[104]N/K[106]S/Y[155]F | 81.5 | 5.2 | 6% | 2 |
| A103N/N105S | A[103]N/N[105]S | 88.0 | 22.5 | 26% | 9 |
| D104N/K106S | D[104]N/K[106]S | 83.2 | 18.2 | 22% | 9 |
| K106N/V108S | K[106]N/V[108]S | 91.9 | 20.2 | 22% | 7 |
| D85N | D[85]N | 64.5 | 21.9 | 34% | 17 |
| T148A | T[148]A | 70.1 | 26.9 | 38% | 44 |
| T148A† | T[148]A† | 74.6 | 16.1 | 22% | 7 |
| K5A | K[5]A | 65.4 | 26.8 | 41% | 4 |
| D64N | D[64]N | 121.4 | 58.8 | 48% | 2 |
| D64A | D[64]A | 129.4 | 36.3 | 28% | 2 |
| N167D | N[167]D | 94.6 | 7.0 | 7% | 2 |
| N167Q | N[167]Q | 77.1 | 35.8 | 46% | 4 |
| S61A | S[61]A | 84.6 | 35.6 | 42% | 4 |
| S53A | S[53]A | 109.9 | 11.6 | 11% | 3 |
| T159A | T[159]A | 100.9 | 1.2 | 1% | 3 |
| T169A | T[169]A | 99.7 | 10.8 | 11% | 3 |
| T172A | T[172]A | 96.2 | 22.1 | 23% | 3 |
| T179A | T[179]A | 94.5 | 16.7 | 18% | 3 |
| Y155H | Y[155]H | 93.9 | 15.8 | 17% | 3 |
| Y155Q | Y[155]Q | 87.6 | 29.8 | 34% | 3 |
| S158A | S[158]A | 107.7 | 0.4 | 0% | 2 |
| S158D | S[158]D | 87.0 | 9.0 | 10% | 2 |
| S158E | S[158]E | 96.0 | 14.1 | 15% | 2 |
| N157Q | N[157]Q | 107.8 | 5.5 | 5% | 2 |
| D203N/F205T | D39N/F41T | 74.3 | 19.5 | 26% | 12 |
| D85N/D203N/F205T | D[85]N/D39N/F41T | 40.6 | 9.1 | 22% | 5 |
| K228N | K63N | 72.5 | 25.5 | 35% | 13 |
| D85N/K228N | D[85]N/K63N | 60.1 | 13.4 | 22% | 6 |
| A103N/N105S/K228N | A[103]N/N[105]S/K63N | 76.5 | 15.8 | 21% | 3 |
| D104N/K106S/K228N | D[104]N/K[106]S/K63N | 96.8 | 21.2 | 22% | 3 |
| Y155F/K228N | Y[155]F/K63N | 73.7 | 3.7 | 5% | 2 |
| D104N/K106S/Y155F/K228N | D[104]N/K[106]S/Y[155] | 76.2 | 6.4 | 8% | 2 |
| | F/K63N | | | | |
| I251S | I86S | 64.3 | 13.3 | 21% | 13 |
| D85N/I251S | D[85]N/I86S | 51.5 | 15.3 | 30% | 5 |
| D85N/D104N/K106S/I251S | D[85]N/D[104]N/K[106] | 46.4 | 19.0 | 41% | 5 |
| | S/I86S | | | | |
| A103N/N105S/I251S | A[103]N/N[105]S/I86S | 90.9 | 41.2 | 45% | 3 |
| D104N/K106S/I251S | D[104]N/K[106]S/I86S | 97.5 | 13.8 | 14% | 2 |
| Y155F/I251S | Y[155]F/I86S | 56.4 | 17.5 | 31% | 2 |
| A262S | A95bS | 99.2 | 19.9 | 20% | 8 |
| K413N | K243N | 106.3 | 40.4 | 38% | 7 |
| E410N | E240N | 45.9 | 19.1 | 42% | 27 |
| E410N* | E240N* | 85.2 | 38.1 | 45% | 10 |
| E239N | E74N | 78.3 | 29.5 | 38% | 9 |
| T241N/H243S | T76N/H78S | 104.5 | 3.5 | 3% | 2 |
| K247N/N249S | K82N/N84S | 75.0 | 15.4 | 21% | 11 |
| Y155F/K247N/N249S | Y[155]F/K82N/N84S | 67.1 | 23.6 | 35% | 4 |
| A103N/N105S/K247N/N249S | A[103]N/N[105]S/K82N/N84S | 84.0 | 9.7 | 12% | 6 |
| D104N/K106S/K247N/N249S | D[104]N/K[106]S/K82N/N84S | 102.3 | 23.0 | 23% | 2 |
| D104N/K106S/Y155F/K247N/ | D[104]N/K[106]S/Y[155] | 89.3 | 10.3 | 12% | 3 |
| N249S | F/K82N/N84S | | | | |
| L321N | L153N | 118.5 | 10.6 | 9% | 2 |
| F314N/H315S | F145N/H147S | 93.0 | 14.3 | 15% | 2 |
| K392N/K394S | K222N/K224S | 0.0 | n.d. | n.d. | 0 |
| S319N/L321S | S151N/L153S | 54.2 | 14.8 | 27% | 3 |
| N260S | N95S | 83.4 | 27.5 | 33% | 13 |
| D104N/K106S/N260S | D[104]N/K[106]S/N95S | 94.3 | 6.8 | 7% | 2 |
| Y155F/N260S | Y[155]F/N95S | 130.6 | 78.1 | 60% | 2 |
| D104N/K106S/Y155F/N260S | D[104]N/K[106]S/Y[155] | 107.7 | 74.8 | 69% | 2 |
| | F/N95S | | | | |
| Y284N | Y117N | 59.8 | 23.5 | 39% | 8 |
| G317N | G149N | 104.6 | n.d. | n.d. | 1 |
| R318N/A320S | R150N/A152S | 84.5 | 21.2 | 25% | 3 |
| R318A | R150A | 62.3 | 28.2 | 45% | 2 |
| R318E | R150E | 33.6 | 10.3 | 31% | 3 |
| R318Y | R150Y | 40.7 | 7.6 | 19% | 3 |
| R312Q | R143Q | 29.9 | 5.0 | 17% | 3 |
| R312A | R143A | 61.6 | 16.9 | 27% | 2 |
| R312Y | R143Y | 27.2 | 11.4 | 42% | 2 |
| R312L | R143L | 28.8 | 0.6 | 2% | 2 |
| V202M | V38M | 40.2 | 1.0 | 2% | 2 |
| V202Y | V38Y | 70.6 | 2.3 | 3% | 2 |
| D203M | D39M | 40.6 | 7.9 | 19% | 5 |
| D203Y | D39Y | 58.0 | 19.5 | 34% | 4 |
| A204M | A40M | 34.0 | 9.2 | 27% | 5 |
| A204Y | A40Y | 39.5 | 10.3 | 26% | 2 |
| K400A/R403A | K230A/R233A | 56.7 | 10.0 | 18% | 2 |
| K400E/R403E | K230E/R233E | 137.1 | 68.4 | 50% | 3 |
| R403A | R233A | 46.4 | 5.2 | 11% | 7 |
| R403E | R233E | 67.0 | 19.4 | 29% | 6 |
| K400A | K230A | 74.6 | 22.1 | 30% | 2 |
| K400E | K230E | 61.3 | 9.3 | 15% | 2 |
| K293E | K126E | 63.2 | 13.9 | 22% | 2 |
| K293A | K126A | 73.7 | 35.2 | 48% | 2 |
| R333A | R165A | 406.7 | 117.5 | 29% | 2 |
| R333E | R165E | 437.3 | n.d. | n.d. | 1 |
| R338A | R170A | 33.7 | 3.7 | 11% | 2 |
| R338E | R170E | 28.7 | 9.0 | 31% | 10 |
| R338A/R403A | R170A/R233A | 73.6 | 18.1 | 25% | 6 |
| R338E/R403E | R170E/R233E | 51.9 | 11.9 | 23% | 2 |
| K293A/R403A | K126A/R233A | 69.2 | 10.2 | 15% | 2 |
| K293E/R403E | K126E/R233E | 104.1 | 31.0 | 30% | 2 |
| K293A/R338A/R403A | K126A/R170A/R233A | 65.4 | 1.3 | 2% | 2 |
| K293E/R338E/R403E | K126E/R170E/R233E | 50.0 | 15.1 | 30% | 2 |
| R318A/R403A | R150A/R233A | 45.7 | 1.6 | 3% | 2 |
| R318E/R403E | R150E/R233E | 75.3 | 47.7 | 63% | 2 |
| R318Y/E410N | R150Y/E240N | 49.6 | 14.3 | 29% | 21 |
| R338E/E410N | R170E/E240N | 12.6 | 3.5 | 28% | 12 |
| R338E/R403E/E410N | R170E/R233E/E240N | 36.7 | 12.2 | 33% | 17 |
| Y155F/R338E/R403E/E410N | Y[155]F/R170E/R233E/E240N | 33.6 | 8.6 | 26% | 2 |
| R318Y/R338E/R403E | R150Y/R170E/R233E | 59.7 | 10.4 | 17% | 3 |
| Y155F/R318Y/R338E/R403E | Y[155]F/R150Y/R170E/R233E | 67.1 | 27.9 | 42% | 2 |
| D203N/F205T/K228N | D39N/F41T/K63N | 39.9 | 3.8 | 9% | 2 |
| D203N/F205T/E410N | D39N/F41T/E240N | 45.5 | 12.0 | 26% | 6 |
| D203N/F205T/R338E | D39N/F41T/R170E | 24.1 | 5.6 | 23% | 2 |
| D203N/F205T/R338A | D39N/F41T/R170A | 38.5 | 9.9 | 26% | 3 |
| D203N/F205T/R318Y | D39N/F41T/R150Y | 47.5 | 6.4 | 13% | 4 |
| D203N/F205T/R338E/R403E | D39N/F41T/R170E/R233E | 51.1 | 10.7 | 21% | 2 |
| K228N/E410N | K63N/E240N | 44.3 | 13.0 | 29% | 10 |
| K228N/R338E | K63N/R170E | 23.1 | 3.0 | 13% | 2 |
| K228N/R338A | K63N/R170A | 31.2 | 4.5 | 14% | 2 |
| K228N/R318Y | K63N/R150Y | 61.3 | 5.4 | 9% | 5 |
| K228N/R338E/R403E | K63N/R170E/R233E | 59.2 | 4.9 | 8% | 2 |
| R403E/E410N | R233E/E240N | 93.7 | 1.0 | 1% | 2 |
| R318Y/R338E/E410N | R150Y/R170E/E240N | 13.9 | 4.0 | 29% | 42 |
| D104N/K106S/R318Y/R338E/ | D[104]N/K[106]S/R150Y/ | 18.9 | 4.1 | 22% | 4 |
| E410N | R170E/E240N | | | | |
| Y155F/R318Y/R338E/E410N | Y[155]F/R150Y/R170E/E240N | 16.0 | 4.8 | 30% | 5 |
| K228N/R318Y/E410N | K63N/R150Y/E240N | 42.0 | 4.7 | 11% | 4 |
| R318Y/R403E/E410N | R150Y/R233E/E240N | 94.2 | 21.1 | 22% | 5 |
| Y155F/R318Y/R403E/E410N | Y[155]F/R150Y/R233E/E240N | 111.4 | 74.7 | 67% | 2 |
| R318Y/R338E/R403E/E410N | R150Y/R170E/R233E/E240N | 43.2 | 13.8 | 32% | 26 |
| A103N/N105S/R318Y/R338E/ | A[103]N/N[105]S/R150Y/ | 44.7 | 20.9 | 47% | 5 |
| R403E/E410N | R170E/R233E/E240N | | | | |
| D104N/K106S/R318Y/R338E/ | D[104]N/K[106]S/R150Y/ | 38.5 | 16.1 | 42% | 3 |
| R403E/E410N | R170E/R233E/E240N | | | | |
| Y155F/R318Y/R338E/R403E/ | Y[155]F/R150Y/R170E/R233E/ | 30.4 | 10.5 | 35% | 4 |
| E410N | E240N | | | | |
| A103N/N105S/Y155F/R318Y/ | A[103]N/N[105]S/Y[155] | 50.7 | 4.5 | 9% | 2 |
| R338E/R403E/E410N | F/R150Y/R170E/R233E/E240N | | | | |
| D104N/K106S/Y155F/R318Y/ | D[104]N/K[106]S/Y[155] | 48.0 | 2.1 | 4% | 2 |
| R338E/R403E/E410N | F/R150Y/R170E/R233E/E240N | | | | |
| D203N/F205T/R318Y/E410N | D39N/F41T/R150Y/E240N | 45.7 | 13.4 | 29% | 6 |
| R333S | R165S | 605.9 | 317.5 | 52% | 3 |
| R338L | R170L | 47.9 | 9.0 | 19% | 3 |
| K316N | K148N | 62.5 | 15.6 | 25% | 3 |
| K316A | K148A | 55.2 | 4.1 | 7% | 3 |
| K316E | K148E | 110.5 | 25.1 | 23% | 3 |
| K316S | K148S | 57.3 | 4.6 | 8% | 3 |
| K316M | K148M | 26.0 | 16.7 | 64% | 3 |
| E239S | E74S | 28.5 | 19.2 | 67% | 3 |
| E239A | E74A | 55.4 | 18.4 | 33% | 3 |
| E239R | E74R | 58.3 | 13.9 | 24% | 3 |
| E239K | E74K | 59.2 | 25.5 | 43% | 3 |
| H257F | H92F | 62.0 | 30.1 | 49% | 3 |
| H257Y | H92Y | 59.3 | 25.0 | 42% | 3 |
| H257E | H92E | 59.7 | 39.6 | 66% | 3 |
| H257S | H92S | 56.0 | 24.7 | 44% | 3 |
| T412A | T242A | 76.1 | 44.7 | 59% | 5 |
| T412V | T242V | 51.2 | 18.9 | 37% | 8 |
| E410N/T412A | E240N/T242A | 37.2 | 3.6 | 10% | 4 |
| E410N/T412V | E240N/T242V | 33.3 | 4.9 | 15% | 4 |
| E410Q | E240Q | 56.1 | 18.0 | 32% | 4 |
| E410S | E240S | 50.0 | 11.9 | 24% | 12 |
| E410A | E240A | 47.7 | 11.7 | 24% | 10 |
| E410D | E240D | 71.9 | 26.9 | 37% | 4 |
| N346D | N178D | 45.7 | 7.8 | 17% | 4 |
| Y155F/N346D | Y[155]F/N178D | 104.4 | 14.5 | 14% | 2 |
| N346Y | N178Y | 27.4 | 4.2 | 15% | 8 |
| Y345A | Y177A | 50.8 | 32.4 | 64% | 4 |
| Y345T | Y177T | 28.6 | 7.9 | 28% | 4 |
| T343R | T175R | 34.5 | 11.8 | 34% | 12 |
| T343E | T175E | 27.3 | 10.0 | 37% | 4 |
| T343Q | T175Q | 37.0 | 9.1 | 25% | 3 |
| F342I | F174I | 30.0 | 19.1 | 64% | 3 |
| T343R/Y345T | T175R/Y177T | 26.5 | 6.8 | 26% | 3 |
| R318Y/R338E | R150Y/R170E | 24.6 | 5.5 | 22% | 4 |
| Y259F/K265T/Y345T | Y94F/K98T/Y177T | 30.9 | 4.8 | 16% | 2 |
| K228N/I251S | K63N/I86S | 122.6 | 53.5 | 44% | 2 |
| K228N/R318Y/R338E/R403E/ | K63N/R150Y/R170E/R233E/ | 36.1 | 14.0 | 39% | 3 |
| E410N | E240N | | | | |
| Y155F/K228N/R318Y/R338E/ | Y[155]F/K63N/R150Y/R170E/ | 40.8 | 15.0 | 37% | 5 |
| R403E/E410N | R233E/E240N | | | | |
| D85N/K228N/R318Y/R338E/ | D[85]N/K63N/R150Y/R170E/ | 39.3 | 9.8 | 25% | 2 |
| R403E/E410N | R233E/E240N | | | | |
| I251S/R318Y/R338E/R403E/ | I86S/R150Y/R170E/R233E/ | 33.4 | 10.2 | 30% | 4 |
| E410N | E240N | | | | |
| D104N/K106S/I251S/R318Y/ | D[104]N/K[106]S/I86S/R150Y/ | 46.2 | 7.7 | 17% | 8 |
| R338E/R403E/E410N | R170E/R233E/E240N | | | | |
| Y155F/I251S/R318Y/R338E/ | D[104]N/K[106]S/I86S/R150Y/ | 43.3 | 7.0 | 16% | 2 |
| R403E/E410N | R170E/R233E/E240N | | | | |
| I251S/R318Y/R338E/E410N | I86S/R150Y/R170E/E240N | 16.1 | 2.7 | 17% | 10 |
| D104N/K106S/I251S/R318Y/ | D[104]N/K[106]S/I86S/R150Y/ | 24.3 | 8.6 | 35% | 3 |
| R338E/E410N | R170E/E240N | | | | |
| F314N/K316S | F145N/K148S | 635.1 | 569.9 | 90% | 2 |
| K247N/N249S/R318Y/R338E/ | K82N/N84S/R150Y/R170E/ | 39.2 | 8.3 | 21% | 6 |
| R403E/E410N | R233E/E240N | | | | |
| Y155F/K247N/N249S/R318Y/ | Y[155]F/K82N/N84S/R150Y/ | 36.3 | 12.8 | 35% | 10 |
| R338E/R403E/E410N | R170E/R233E/E240N | | | | |
| A103N/N105S/K247N/N249S/ | A[103]N/N[105]S/K82N/N84S/ | 28.0 | 9.5 | 34% | 6 |
| R318Y/R338E/R403E/E410N | R150Y/R170E/R233E/E240N | | | | |
| D104N/K106S/K247N/N249S/ | D[104]N/K[106]S/K82N/N84S/ | 59.0 | 0.6 | 1% | 2 |
| R318Y/R338E/R403E/E410N | R150Y/R170E/R233E/E240N | | | | |
| D104N/K106S/Y155F/K247N/ | D[104]N/K[106]S/Y[155] | 51.8 | 16.7 | 32% | 6 |
| N249S/R318Y/R338E/R403E/ | F/K82N/N84S/R150Y/R170E/ | | | | |
| E410N | R233E/E240N | | | | |
| K247N/N249S/R318Y/R338E/ | K82N/N84S/R150Y/R170E/ | 16.6 | 3.7 | 22% | 6 |
| E410N | E240N | | | | |
| Y155F/K247N/N249S/R318Y/ | Y[155]F/K82N/N84S/R150Y/ | 14.7 | 3.9 | 27% | 9 |
| R338E/E410N | R170E/E240N | | | | |
| R318Y/R338E/R403E/E410S | R150Y/R170E/R233E/E240S | 36.4 | 9.5 | 26% | 7 |
| R318Y/R338E/E410S | R150Y/R170E/E240S | 16.4 | 4.0 | 25% | 8 |
| K228N/K247N/N249S | K63N/K82N/N84S | 94.5 | 27.0 | 29% | 2 |
| D104N/K106S/Y155F/K228N/ | D[104]N/K[106]S/Y[155] | 75.3 | 26.4 | 35% | 2 |
| K247N/N249S | F/K63N/K82N/N84S | | | | |
| D104N/K106S/K228N/K247N/ | D[104]N/K[106]S/K63N/K82N/ | 77.1 | 18.3 | 24% | 5 |
| N249S | N84S | | | | |
| Y155F/K228N/K247N/N249S | Y[155]F/K63N/K82N/N84S | 79.2 | 27.6 | 35% | 2 |
| K228N/K247N/N249S/R318Y/ | K63N/K82N/N84S/R150Y/R170E/ | 49.7 | 15.6 | 31% | 17 |
| R338E/R403E/E410N | R233E/E240N | | | | |
| D104N/K106S/K228N/K247N/ | D[104]N/K[106]S/K63N/K82N/ | 53.3 | 12.2 | 23% | 7 |
| N249S/R318Y/R338E/R403E/ | N84S/R150Y/R170E/R233E/E240N | | | | |
| E410N | | | | | |
| Y155F/K228N/K247N/N249S/ | Y[155]F/K63N/K82N/N84S/ | 45.4 | 17.7 | 39% | 5 |
| R318Y/R338E/R403E/E410N | R150Y/R170/R233E/E240N | | | | |
| R318Y/R338E/R403E/E410N/ | R150Y/R170E/R233E/E240N/ | 48.3 | 16.2 | 33% | 6 |
| T412V | T242V | | | | |
| R318Y/R338E/R403E/E410N/ | R150Y/R170E/R233E/E240N/ | 34.4 | 10.0 | 29% | 6 |
| T412A | T242A | | | | |
| R318Y/R338E/R403E/T412A | R150Y/R170E/R233E/T242A | 67.5 | 11.6 | 17% | 4 |
| R318Y/R338E/T412A | R150Y/R170E/T242A | 23.5 | 5.3 | 22% | 6 |
| R318Y/R338E/E410N/T412V | R150Y/R170E/E240N/T242V | 23.6 | 12.3 | 52% | 11 |
| N260S/R318Y/R338E/R403E/ | N95S/R150Y/R170E/R233E/ | 72.4 | 20.2 | 28% | 2 |
| E410N | E240N | | | | |
| D104N/K106S/N260S/R318Y/ | D[104]N/K[106]S/N95S/R150Y/ | 61.1 | 0.0 | 0% | 2 |
| R338E/R403E/E410N | R170E/R233E/E240N | | | | |
| Y155F/N260S/R318Y/R338E/ | Y[155]F/N95S/R150Y/R170E/ | 83.9 | 4.4 | 5% | 2 |
| R403E/E410N | R233E/E240N | | | | |
| R318Y/R338E/N346D/R403E/ | R150Y/R170E/N178D/R233E/ | 77.7 | 20.9 | 27% | 2 |
| E410N | E240N | | | | |
| Y155F/R318Y/R338E/N346D/ | Y[155]F/R150Y/R170E/N178D/ | 100.0 | 15.6 | 16% | 2 |
| R403E/E410N | R233E/E240N | | | | |
| K247N/N249S/N260S | K82N/N84S/N95S | 114.1 | 0.0 | 0% | 2 |
| Y155F/K247N/N249S/N260S | Y[155]F/K82N/N84S/N95S | 96.5 | 5.5 | 6% | 2 |
| D104N/K106S/K247N/N249S/ | D[104]N/K[106]S/K82N/ | 61.2 | 14.1 | 23% | 2 |
| N260S | N84S/N95S | | | | |
| D104N/K106S/Y155F/K247N/ | D[104]N/K[106]S/Y[155] | 68.5 | 33.2 | 49% | 2 |
| N249S/N260S | F/K82N/N84S/N95S | | | | |
| K247N/N249S/N260S/R318Y/ | K82N/N84S/N95S/R150Y/ | 47.4 | 12.1 | 26% | 6 |
| R338E/R403E/E410N | R170E/R233E/E240N | | | | |
| Y155F/K247N/N249S/N260S/ | Y[155]F/K82N/N84S/N95S/ | 95.4 | 73.0 | 77% | 5 |
| R318Y/R338E/R403E/E410N | R150Y/R170E/R233E/E240N | | | | |
| Y155F/N260S/N346D | Y[155]F/N95S/N178D | 127.9 | 6.2 | 5% | 2 |
| R318Y/R338E/T343R/R403E/ | R150Y/R170E/T175R/R233E/ | 24.7 | 7.2 | 29% | 13 |
| E410N | E240N | | | | |
| Y155F/R318Y/R338E/T343R/ | Y[155]F/R150Y/R170E/ | 27.2 | 5.7 | 21% | 4 |
| R403E/E410N | T175R/R233E/E240N | | | | |
| D104N/K106S/R318Y/R338E/ | D[104]N/K[106]S/R150Y/ | 26.6 | 5.0 | 19% | 5 |
| T343R/R403E/E410N | R170E/T175R/R233E/E240N | | | | |
| R338E/T343R | R170E/T175R | 14.3 | 3.6 | 25% | 7 |
| T343R/N346Y | T175R/N178Y | 26.0 | 7.3 | 28% | 11 |
| R318Y/R338E/N346Y/R403E/ | R150Y/R170E/N178Y/R233E/ | 28.1 | 7.5 | 27% | 3 |
| E410N | E240N | | | | |
| R318Y/R338E/T343R/N346Y/ | R150Y/R170E/T175R/N178Y/ | 15.8 | 4.0 | 25% | 5 |
| R403E/E410N | R233E/E240N | | | | |
| T343R/N346D | T175R/N178D | 118.5 | 42.9 | 36% | 2 |
| R318Y/R338E/T343R/N346D/ | R150Y/R170E/T175R/N178D/ | 67.0 | 26.8 | 40% | 2 |
| R403E/E410N | R233E/E240N | | | | |
| R318Y/R338E/Y345A/R403E/ | R150Y/R170E/Y177A/R233E/ | 18.8 | 8.8 | 47% | 6 |
| E410N | E240N | | | | |
| R318Y/R338E/Y345A/N346D/ | R150Y/R170E/Y177A/N178D/ | 56.5 | 16.1 | 28% | 3 |
| R403E/E410N | R233E/E240N | | | | |
| Y155F/K247N/N249S/R318Y/ | Y[155]F/K82N/N84S/R150Y/ | 67.3 | 17.7 | 26% | 5 |
| R338E/R403E | R170E/R233E | | | | |
| K247N/N249S/R318Y/R338E/ | K82N/N84S/R150Y/R170E/ | 53.6 | 22.1 | 41% | 2 |
| R403E | R233E | | | | |
| Y155F/K247N/N249S/R318Y/ | Y[155]F/K82N/N84S/R150Y/ | 125.4 | 9.1 | 7% | 3 |
| R403E/E410N | R233E/E240N | | | | |
| K247N/N249S/R318Y/R403E/ | K82N/N84S/R150Y/R233E/ | 110.9 | 29.5 | 27% | 10 |
| E410N | E240N | | | | |
| Y155F/K247N/N249S/R338E/ | Y[155]F/K82N/N84S/R170E/ | 48.7 | 11.4 | 23% | 3 |
| R403E/E410N | R233E/E240N | | | | |
| K247N/N249S/R338E/R403E/ | K82N/N84S/R170E/R233E/ | 25.0 | 7.9 | 31% | 2 |
| E410N | E240N | | | | |
| R318Y/R338E/T343R/R403E | R150Y/R170E/T175R/R233E | 44.3 | 11.0 | 25% | 4 |
| Y155F/R318Y/R338E/T343R/ | Y[155]F/R150Y/R170E/ | 34.0 | 8.7 | 26% | 4 |
| R403E | T175R/R233E | | | | |
| R318Y/R338E/T343R/E410N | R150Y/R170E/T175R/E240N | 16.4 | 5.9 | 36% | 16 |
| Y155F/R318Y/R338E/T343R/ | Y[155]F/R150Y/R170E/ | 25.6 | 5.4 | 21% | 4 |
| E410N | T175R/E240N | | | | |
| R318Y/T343R/R403E/E410N | R150Y/T175R/R233E/ | 93.9 | 14.0 | 15% | 3 |
| | E240N | | | | |
| Y155F/R318Y/T343R/R403E/ | Y[155]F/R150Y/T175R/ | 34.0 | 7.7 | 23% | 2 |
| E410N | R233E/E240N | | | | |
| R338E/T343R/R403E/E410N | R170E/T175R/R233E/E240N | 34.7 | 14.3 | 41% | 2 |
| Y155F/R338E/T343R/R403E/ | Y[155]F/R170E/T175R/ | 25.9 | 8.2 | 32% | 4 |
| E410N | R233E/E240N | | | | |
| Y155F/K247N/N249S/R318Y/ | Y[155]F/K82N/N84S/ | 25.7 | 8.4 | 33% | 11 |
| R338E/T343R/R403E/E410N | R150Y/R170E/T175R/ | | | | |
| | R233E/E240N | | | | |
| K247N/N249S/R318Y/R338E/ | K82N/N84S/R150Y/R170E/ | 29.2 | 7.9 | 27% | 5 |
| T343R/R403E/E410N | T175R/R233E/E240N | | | | |
| K228N/I251S/R318Y/R338E/ | K63N/I86S/R150Y/R170E/ | 36.4 | 10.8 | 30% | 7 |
| R403E/E410N | R233E/E240N | | | | |
| Y155F/K228N/I251S/R318Y/ | Y[155]F/K63N/I86S/R150Y/ | 39.3 | 7.3 | 19% | 5 |
| R338E/R403E/E410N | R170E/R233E/E240N | | | | |
| N260S/R318Y/R338E/T343R/ | N95S/R150Y/R170E/T175R/ | 32.1 | 10.3 | 32% | 7 |
| R403E/E410N | R233E/E240N | | | | |
| Y155F/N260S/R318Y/R338E/ | Y[155]F/N95S/R150Y/ | 40.2 | 11.6 | 29% | 5 |
| T343R/R403E/E410N | R170E/T175R/R233E/E240N | | | | |
| K228N/K247N/N249S/R318Y/ | K63N/K82N/N84S/R150Y/ | 25.1 | 5.4 | 21% | 12 |
| R338E/T343R/R403E/E410N | R170E/T175R/R233E/E240N | | | | |
| Y155F/K228N/K247N/N249S/ | Y[155]F/K63N/K82N/N84S/ | 36.8 | 18.8 | 51% | 5 |
| R318Y/R338E/T343R/R403E/ | R150Y/R170E/T175R/R233E/ | | | | |
| E410N | E240N | | | | |
| Y155F/R338E/T343R/R403E | Y[155]F/R170E/T175R/R233E | 28.9 | 9.1 | 31% | 5 |
| R338E/T343R/R403E | R170E/T175R/R233E | 23.5 | 6.5 | 28% | 2 |
| Y155F/R338E/T343R/R403E/ | Y[155]F/R170E/T175R/ | 23.9 | 3.1 | 13% | 6 |
| E410S | R233E/E240S | | | | |
| Y155F/N260S/R338E/T343R/ | Y[155]F/N95S/R170E/ | 69.2 | 27.8 | 40% | 6 |
| R403E | T175R/R233E | | | | |
| Y155F/I251S/R338E/T343R/ | Y[155]F/I86S/R170E/ | 19.6 | 3.4 | 17% | 2 |
| R403E | T175R/R233E | | | | |
| R318Y/R338E/T343R/R403E/ | R150Y/R170E/T175R/ | 19.0 | 6.4 | 33% | 14 |
| E410S | R233E/E240S | | | | |
| Y155F/K247N/N249S/T343R/ | Y[155]F/K82N/N84S/ | 59.6 | 20.3 | 34% | 4 |
| R403E | T175R/R233E | | | | |
| Y155F/K247N/N249S/R318Y/ | Y[155]F/K82N/N84S/ | 36.5 | 3.5 | 10% | 2 |
| R338E/T343R/R403E | R150Y/R170E/T175R/R233E | | | | |
| K247N/N249S/R318Y/R338E/ | K82N/N84S/R150Y/R170E/ | 28.4 | 17.8 | 63% | 4 |
| T343R/R403E | T175R/R233E | | | | |
| Y155F/K247N/N249S/R338E/ | Y[155]F/K82N/N84S/R170E/ | 26.4 | 1.3 | 5% | 2 |
| T343R/R403E/E410N | T175R/R233E/E240N | | | | |
| K247N/N249S/R338E/T343R/ | K82N/N84S/R170E/T175R/ | 25.1 | 3.0 | 12% | 2 |
| R403E/E410N | R233E/E240N | | | | |
| Y155F/K247N/N249S/R318Y/ | Y[155]F/K82N/N84S/R150Y/ | 26.3 | 8.8 | 33% | 2 |
| R338E | R170E | | | | |
| Y155F/K247N/N249S/R318Y/ | Y[155]F/K82N/N84S/R150Y/ | 42.1 | 12.8 | 30% | 4 |
| T343R | T175R | | | | |
| Y155F/K247N/N249S/R318Y/ | Y[155]F/K82N/N84S/R150Y/ | 108.6 | 22.3 | 21% | 3 |
| R403E | R233E | | | | |
| Y155F/K247N/N249S/R318Y/ | Y[155]F/K82N/N84S/R150Y/ | 48.8 | 12.8 | 26% | 3 |
| E410N | E240N | | | | |
| Y155F/K247N/N249S/R338E/ | Y[155]F/K82N/N84S/R170E/ | 40.9 | 12.9 | 31% | 2 |
| R403E | R233E | | | | |
| Y155F/K247N/N249S/R338E/ | Y[155]F/K82N/N84S/R170E/ | 15.3 | 4.0 | 26% | 2 |
| T343R | T175R | | | | |
| Y155F/K247N/N249S/R318Y/ | Y[155]F/K82N/N84S/R150Y/ | 17.7 | 6.0 | 34% | 4 |
| R338E/T343R/E410N | R170E/T175R/E240N | | | | |
| K247N/N249S/R318Y/R338E/ | K82N/N84S/R150Y/R170E/ | 32.8 | 22.9 | 70% | 6 |
| T343R/E410N | T175R/E240N | | | | |
| Y155F/K247N/N249S/R318Y/ | Y[155]F/K82N/N84S/R150Y/ | 60.6 | 26.0 | 43% | 2 |
| T343R/R403E/E410N | T175R/R233E/E240N | | | | |
| K247N/N249S/R318Y/T343R/ | K82N/N84S/R150Y/T175R/ | 80.5 | 31.3 | 39% | 7 |
| R403E/E410N | R233E/E240N | | | | |
| Y155F/K247N/N249S/R338E/ | Y[155]F/K82N/N84S/R170E/ | 17.7 | 7.6 | 43% | 8 |
| E410N | E240N | | | | |
| Y155F/K247N/N249S/R318Y/ | Y[155]F/K82N/N84S/R150Y/ | 60.5 | 7.5 | 12% | 2 |
| T343R/R403E | T175R/R233E | | | | |
| K247N/N249S/R318Y/T343R/ | K82N/N84S/R150Y/T175R/ | 105.3 | 25.8 | 25% | 9 |
| R403E | R233E | | | | |
| Y155F/K247N/N249S/R318Y/ | Y[155]F/K82N/N84S/ | 38.1 | 29.6 | 78% | 4 |
| T343R/E410N | R150Y/T175R/E240N | | | | |
| K247N/N249S/R318Y/T343R/ | K82N/N84S/R150Y/T175R/ | 40.1 | 25.9 | 64% | 4 |
| E410N | E240N | | | | |
| Y155F/K247N/N249S/R338E/ | Y[155]F/K82N/N84S/ | 25.1 | 2.8 | 11% | 2 |
| T343R/R403E | R170E/T175R/R233E | | | | |
| K247N/N249S/R338E/T343R/ | K82N/N84S/R170E/T175R/ | 26.3 | 3.5 | 13% | 2 |
| R403E | R233E | | | | |
| Y155F/K247N/N249S/R338E/ | Y[155]F/K82N/N84S/ | 27.0 | 7.1 | 26% | 2 |
| T343R/E410N | R170E/T175R/E240N | | | | |
| K247N/N249S/R338E/T343R/ | K82N/N84S/R170E/T175R/ | 27.5 | 11.1 | 40% | 5 |
| E410N | E240N | | | | |
| Y155F/K247N/N249S/T343R/ | Y[155]F/K82N/N84S/ | 52.0 | 5.4 | 10% | 2 |
| R403E/E410N | T175R/R233E/E240N | | | | |
| K247N/N249S/T343R/R403E/ | K82N/N84S/T175R/R233E/ | 60.0 | 13.9 | 23% | 2 |
| E410N | E240N | | | | |
| Y155F/R318Y/R338E/T343R | Y[155]F/R150Y/R170E/ | 24.2 | 8.8 | 36% | 7 |
| | T175R | | | | |
| R318Y/R338E/T343R | R150Y/R170E/T175R | 30.0 | 1.5 | 5% | 2 |
| Y155F/R318Y/T343R/R403E | Y[155]F/R150Y/T175R/ | 72.7 | 29.5 | 41% | 2 |
| | R233E | | | | |
| Y155F/T343R/R403E/E410N | Y[155]F/T175R/R233E/ | 44.6 | 1.9 | 4% | 2 |
| | E240N | | | | |
| Y155F/K247N/N249S/R318Y/ | Y[155]F/K82N/N84S/ | 27.6 | 13.2 | 48% | 7 |
| R338E/T343R | R150Y/R170E/T175R | | | | |
| K247N/N249S/R318Y/R338E/ | K82N/N84S/R150Y/R170E/ | 24.4 | 13.5 | 55% | 4 |
| T343R | T175R | | | | |
| Y155F/K247N/N249S/T343R/ | Y[155]F/K82N/N84S/ | 34.4 | 20.0 | 58% | 5 |
| E410N | T175R/E240N | | | | |
| Y155F/K247N/N249S/R403E/ | Y[155]F/K82N/N84S/ | 131.3 | 53.1 | 40% | 7 |
| E410N | R233E/E240N | | | | |
| Y155F/R338E/T343R/E410N | Y[155]F/R170E/T175R/ | 22.4 | 13.8 | 62% | 6 |
| | E240N | | | | |
| R338E/T343R/E410N | R170E/T175R/E240N | 35.5 | 15.9 | 45% | 2 |
| Y155F/R318Y/T343R/E410N | Y[155]F/R150Y/T175R/ | 40.3 | 22.9 | 57% | 4 |
| | E240N | | | | |
| R318Y/T343R/E410N | R150Y/T175R/E240N | 52.3 | 2.4 | 5% | 2 |
| K228N/R318Y/R338E/T343R/ | K63N/R150Y/R170E/ | 40.3 | 9.6 | 24% | 3 |
| R403E/E410N | T175R/R233E/E240N | | | | |
| K228N/K247N/N249S/R318Y/ | K63N/K82N/N84S/R150Y/ | 44.4 | 23.7 | 53% | 3 |
| R338E/T343R/R403E | R170E/T175R/R233E | | | | |
| K228N/247N/N249S/R318Y/ | K63N/K82N/N84S/R150Y/ | 38.1 | 10.4 | 27% | 2 |
| R338E/T343R/E410N | R170E/T175R/E240N | | | | |
| K228N/K247N/N249S/R318Y/ | K63N/K82N/N84S/R150Y/ | 125.1 | 36.4 | 29% | 3 |
| T343R/R403E/E410N | T175R/R233E/E240N | | | | |

†produced in BHK-21 cells;
*80% glycosylated form of E410N

### Example 5

### Determination of the Inhibition of FIXa by the Antithrombin/Heparin Complex

Inhibition of wild-type FIXa or FIXa variants by the Antithrombin/heparin complex (AT-III/heparin) was assessed by measuring the level of inhibition by various concentrations of AT-III/heparin on the catalytic activity of FIXa towards a small molecule substrate, Mesyl-D-CHG-Gly-Arg-AMC (Pefafluor FIXa; Pentapharm). A K_{0.5} value is determined for each FIXa variant tested, which corresponds to the molar concentration of AT-III that was required for 50% inhibition (IC₅₀) of the catalytic activity of a FIXa variant under the predefined conditions of the assay. Inhibition reactions were performed in the presence of low molecular weight heparin (LMWH; Calbiochem) or full-length unfractionated heparin (UFH; Calbiochem), the latter requiring modified protocol conditions to account for an increase in the rate of inhibition. The apparent second-order rate constant (kₐₚₚ) for the inhibition of wild-type FIXa or FIXa variants by the AT-III/UFH complex was also directly evaluated using a modified protocol, in which the time of incubation with the AT-III/UFH complex was varied.

### A. Inhibition of FIXa by the Antithrombin/LMWH Complex

For inhibition reactions in the presence of LMWH, a 200 nM solution of AT-III/LMWH (final 2 μM LMWH) was prepared by dilution of a 20 μM stock of plasma purified human AT-III (Molecular Innovations) into a solution of 2 μM LMWH in a 1.2 mL volume of 1× Buffer A (50 mM Tris, 100 mM NaCl, 10 mM CaCl₂), 0.01% Tween-20, pH 7.4). This solution of AT-III/LMWH was for use as the highest concentration in the assay. AT-III/LMWH solutions were incubated for at least 30 minutes at room temperature and then serially diluted 1.5-fold in a 96 deep-well polypropylene plate with a final volume of 400 μL 1× Buffer A that contained 2 μM LMWH, resulting in dilutions of 200 nM, 133.3, nM 88.9 nM, 59.3 nM, 39.5 nM, 26.3 nM, 17.6 nM and 0 nM (i.e., rows A-H). A total of 25 μL was aliquoted into their respective rows of a 96-well V-bottom storage plate to fill all columns (i.e. 1-12). FIXa variants were initially diluted to 100 nM in 1× Buffer A. Subsequently, 36 μL of each 100 nM FIXa variant was diluted to a concentration of 1.8 nM in 2.0 mL of 1× Buffer A and then 60 μL of this solution was aliquoted into a 96-well V-bottom storage plate according to a predefined plate map (4 FIXa variants per plate).

Assay reactions were initiated using a BioMek FX liquid handling system programmed to dispense 25 μL of the FIXa solutions into the plates containing 25 of each dilution of AT-III/LMWH per well for a total of two duplicate assay plates for each FIXa variant. The final inhibition assay conditions were: 0.9 nM FIXa and AT-III dilutions ranging from 0 to 100 nM in 1 μM LMWH. Inhibition reactions were further incubated for 1 minute at room temperature (˜25° C.) before a 25 μL aliquot of the reaction was transferred by the BioMek FX to a 96-well black half-area plate containing 25 μL of 1.6 mM Mesyl-D-CHG-Gly-Arg-AMC per well in assay Buffer B (50 mM Tris, 100 mM NaCl, 10 mM CaCl₂), 0.01% Tween-20, pH 7.4, 60% ethylene glycol). Polybrene (hexadimethrine bromide) at a final concentration of 5 mg/mL was added in Buffer B to quench the AT-III/LMWH reaction. Residual activity of FIXa was assessed by following the initial rates of substrate cleavage for 60 minutes in a fluorescence reader set to 25° C. The final assay conditions for determination of residual activity are 0.45 nM FIXa variant, 0.8 mM Mesyl-D-CHG-Gly-Arg-AMC, 30% ethylene glycol and 5 mg/mL polybrene in 50 mM Tris, 100 mM NaCl, 10 mM CaCl₂), 0.01% Tween-20, pH 7.4.

To determine the degree of inhibition by AT-III/LMWH for FIXa or FIXa variants, raw data collected with the SoftMax Pro application (Molecular Devices) were exported as .XML files. Further non-linear data analyses were performed with XLfit4, a software package for automated curve fitting and statistical analysis within the Microsoft Excel spreadsheet environment (IDBS Software) or directly within the ActivityBase software package using the XE Runner data analysis module (IDBS Software). The template was used to calculate the AT-III dilution series, ratio of AT-III to FIXa, and the Vi/Vo ratios for each FIXa replicate at each experimental AT-III concentration. The spreadsheet template was used to calculate the AT-III dilution series, ratio of AT-III to FIXa, and the Vi/Vo ratios for each FIXa replicate at each experimental AT-III concentration. Non-linear regression analyses of residual FIXa activity (expressed as Vi/Vo) versus AT-III concentration was processed using XLfit4 and a hyperbolic inhibition equation of the form ((C+(Amp*(1−(X/(K_{0.5}+X))))); where C=the offset (fixed at 0 to permit extrapolation of data sets that did not reach 100% inhibition during the course of the assay), Amp=the amplitude of the fit and K_{0.5}, which corresponds to the concentration of AT-III required for half-maximal inhibition under the assay conditions. For several FIXa variants, AT-III/LMWH inhibited less than 10-15% of the total protease activity at the highest tested concentration of AT-III, representing an upper limit of detection for the assay under standard screening conditions. Variants with less than 10% maximal inhibition were therefore assigned a lower limit K_{0.5} value of 999 nM and in most cases are expected to have AT-III resistances much greater than the reported value.

Table 29 provides the results of the assays that were performed using AT-III/LMWH. The results are presented both as the fitted K_{0.5} parameter and as a representation of the extent of AT-III resistance for each variant compared to the wild-type FIXa expressed as a ratio of their fitted K_{0.5} values (K_{0.5} variant/K_{0.5} wild-type). Where the K_{0.5} parameter of the FIXa variant was compared to wild-type FIXa, it was compared to a recombinant wild-type FIXa polypeptide that was expressed and purified using the same conditions as used for the variant FIXa polypeptides to ensure that any differences in activity were the result of the mutation(s), and not the result of differences in, for example, post-translational modifications associated with different expression systems. Thus, the wild-type FIXa polypeptide used for comparison was the recombinant wild-type FIXa generated from cloning the FIX gene set forth in SEQ ID NO:1 and expressed from CHOX cells as a polypeptide with an amino acid sequence set forth in SEQ ID NO:3, as described in Example 1 (i.e., Catalyst Biosciences WT FIX polypeptide). Several FIXa variants exhibited greater than 20-fold increased resistance to AT-III compared to wild-type FIXa (Catalyst Biosciences WT FIXa). For example, FIXa-R318A/R403A, FIXa-R318E/R340E, FIXa-R318A, FIXa-R318E, FIXa-K400E, FIXa-R338E/R403E and FIXa-K400A/R403A are among the group that exhibited significant resistance to AT-III.

TABLE 29
Inhibition of FIXa variants by AT-III/LMWH

| Mutation | Mutation | K_{0.5} | ±S.D. | | K_{0.5-mut}/ | |
|---|---|---|---|---|---|---|
| (Mature FIX Numbering) | (Chymotrypsin Numbering) | (nM) | (nM) | % CV | K_{0.5-wt} | n |
| Plasma Purified FIXa | Plasma Purified FIXa | 20.2 | 6.7 | 33% | 0.7 | 3 |
| BeneFIX ® (T148A) | BeneFIX ® (T[148]A) | 27.3 | 4.7 | 17% | 0.9 | 2 |
| Catalyst Biosciences WT | Catalyst Biosciences WT | 29.4 | 7.3 | 25% | 1.0 | 10 |
| A103N/N105S | A[103]N/N[105]S | 31.1 | n/a | n/a | 1.1 | 1 |
| D104N/K106S | D[104]N/K[106]S | 26.1 | n/a | n/a | 0.9 | 1 |
| K106N/V108S | K[106]N/V[108]S | 47.7 | n/a | n/a | 1.6 | 1 |
| D85N | D[85]N | 33.1 | n/a | n/a | 1.1 | 1 |
| T148A | T[148]A | 22.9 | 1.7 | 8% | 0.8 | 4 |
| D203N/F205T | D39N/F41T | 154.1 | 50.1 | 33% | 5.2 | 4 |
| I251S | I86S | 22.6 | n/a | n/a | 0.8 | 1 |
| D85N/I251S | D[85]N/I86S | 28.3 | n/a | n/a | 1.0 | 1 |
| D85N/D104N/K106S/I251S | D[85]N/D[104]N/K[106]S/I86S | 32.1 | n/a | n/a | 1.1 | 1 |
| A262S | A95bS | 25.3 | n/a | n/a | 0.9 | 1 |
| K413N | K243N | 34.2 | n/a | n/a | 1.2 | 1 |
| E410N | E240N | 24.8 | 7.8 | 31% | 0.8 | 3 |
| E239N | E74N | 191.8 | 61.0 | 32% | 6.5 | 3 |
| T241N/H243S | T76N/H78S | 35.4 | n/a | n/a | 1.2 | 1 |
| K247N/N249S | K82N/N84S | 23.1 | n/a | n/a | 0.8 | 1 |
| L321N | L153N | 39.0 | n/a | n/a | 1.3 | 1 |
| F314N/H315S | F145N/H147S | 191.8 | 59.8 | 31% | 6.5 | 3 |
| S319N/L321S | S151N/L153S | 113.4 | n/a | n/a | 3.9 | 1 |
| N260S | N95S | 64.6 | n/a | n/a | 2.2 | 1 |
| Y284N | Y117N | 36.7 | n/a | n/a | 1.2 | 1 |
| R318A | R150A | 896.2 | 189.2 | 21% | 30.5 | 2 |
| R318E | R150E | 861.1 | 21.8 | 3% | 29.3 | 2 |
| R318Y | R150Y | 395.1 | 6.3 | 2% | 13.5 | 2 |
| R312Q | R143Q | 52.7 | 5.1 | 10% | 1.8 | 2 |
| R312A | R143A | 51.9 | 1.3 | 3% | 1.8 | 2 |
| R312Y | R143Y | 323.0 | 13.7 | 4% | 11.0 | 2 |
| R312L | R143L | 25.5 | 2.9 | 11% | 0.9 | 2 |
| V202M | V38M | 20.3 | 5.1 | 25% | 0.7 | 2 |
| V202Y | V38Y | 27.2 | 6.9 | 25% | 0.9 | 2 |
| D203M | D39M | 18.6 | 6.9 | 37% | 0.6 | 2 |
| D203Y | D39Y | 31.1 | 0.3 | 1% | 1.1 | 2 |
| A204M | A40M | 45.8 | 11.1 | 24% | 1.6 | 2 |
| A204Y | A40Y | 43.4 | 22.3 | 51% | 1.5 | 2 |
| K400A/R403A | K230A/R233A | 585.0 | 160.5 | 27% | 19.9 | 2 |
| K400E/R403E | K230E/R233E | 299.0 | 206.5 | 69% | 10.2 | 2 |
| R403A | R233A | 164.3 | 88.7 | 54% | 5.6 | 2 |
| R403E | R233E | 264.2 | 80.9 | 31% | 9.0 | 2 |
| K400A | K230A | 384.0 | 121.1 | 32% | 13.1 | 2 |
| K400E | K230E | 614.8 | 71.4 | 12% | 20.9 | 2 |
| K293E | K126E | 290.2 | 42.1 | 15% | 9.9 | 2 |
| K293A | K126A | 194.1 | 38.0 | 20% | 6.6 | 2 |
| R333A | R165A | 225.7 | 72.7 | 32% | 7.7 | 2 |
| R333E | R165E | 345.6 | 1.7 | 0% | 11.8 | 2 |
| R338A | R170A | 56.2 | 8.4 | 15% | 1.9 | 2 |
| R338E | R170E | 238.4 | n/a | n/a | 8.1 | 1 |
| R338A/R403A | R170A/R233A | 418.5 | 150.9 | 36% | 14.2 | 2 |
| R338E/R403E | R170E/R233E | 601.6 | 241.5 | 40% | 20.5 | 2 |
| K293A/R403A | K126A/R233A | 486.3 | 114.9 | 24% | 16.6 | 2 |
| K293E/R403E | K126E/R233E | 342.0 | 4.9 | 1% | 11.6 | 2 |
| K293A/R338A/R403A | K126A/R170A/R233A | 497.1 | 85.9 | 17% | 16.9 | 2 |
| K293E/R338E/R403E | K126E/R170E/R233E | 418.5 | 150.9 | 36% | 14.2 | 2 |
| R318A/R403A | R150A/R233A | 999.0 | n/a | n/a | 34.0 | 2 |
| R318E/R403E | R150E/R233E | 999.0 | n/a | n/a | 34.0 | 2 |

* A K_{0.5} value of 999 nM indicates the lower limit value for those variants with less than 10% inhibition under the conditions of the assay.

### B. Inhibition of FIXa by the Antithrombin/UFH Complex

Additional experiments were performed to assess the inhibition of FIXa variants by AT-III/UFH (unfractionated full-length heparin) using the same assay as described above with minor modifications. Full-length, unfractionated heparin (Calbiochem) was used instead of low molecular weight heparin (LMWH) to observe the effects of FIXa variant mutations on the increased rate of the inhibition reaction due to the “templating” effect provided by longer heparin chains (see e.g., Olson et al. (2004) J. Thromb. Haemost. 92(5), 929-939).

For inhibition reactions in the presence of UFH, a 70 nM, 600 nM, 2000 nM, 6000 nM or 10000 nM solution of AT-III/UFH (final 1 μM UFH) was prepared by dilution of a 20 μM stock of plasma purified human AT-III (Molecular Innovations) into a solution of excess UFH (2 to 20 μM) in a 1.4 mL volume of 1× Buffer A (50 mM Tris, 100 mM NaCl, 10 mM CaCl₂), 0.01% Tween-20, pH 7.4). AT-III/UFH solutions were also incubated for 30 minutes at room temperature before being serially diluted 1.5-fold in a 96 deep-well polypropylene plate with a final volume of 460 μL 1× Buffer A containing 1 μM UFH. The final dilutions of AT-III for the modified assay were dependent on the starting concentration of AT-III and ranged from 70 nM-0 nM, 600 nM-0 nM, 100 nM-0 nM or 5000 nM-0 nM (i.e., rows A-H). Those variants, which showed increased resistance to AT-III inhibition under the standard conditions, were further tested using higher concentrations of AT-III. A total of 35 μL of each AT-III dilution was aliquoted into their respective rows of a 96-well V-bottom storage plate to fill all columns (i.e., 1-12). FIXa variants were initially diluted to 100 nM in 1× Buffer A. Subsequently, 15 μL of each 100 nM FIXa variant was diluted to a concentration of 0.6 nM in 2.0 mL of 1× Buffer A and then 70 μL of this solution was aliquoted into a 96-well V-bottom storage plate according to the same predefined plate map (4 FIXa variants per plate).

Assay reactions were initiated using a BioMek FX liquid handling system programmed to dispense 35 μL of the FIXa solutions into the plates containing 35 of each dilution of AT-III/heparin per well for a total of two duplicate assay plates for each FIXa variant. The final inhibition assay conditions were: 0.3 nM FIXa and AT-III dilutions ranging from 35 nM to 0 nM, 300 nM to 0 nM, 1000 nM to 0 nM, 3000 nM to 0 nM or 5000 nM to 0 nM in UFH ranging from 1 μM to 10 μM, depending of the highest AT-III concentration so that the heparin remained in excess. Inhibition reactions were further incubated for 10 seconds at room temperature (˜25° C.) before a 40 μL aliquot of the reaction was transferred by the BioMek FX to a 96-well black half-area plate containing 20 μL of 2.5 mM Mesyl-D-CHG-Gly-Arg-AMC per well in assay Buffer C (50 mM Tris, 100 mM NaCl, 10 mM CaCl₂), 0.01% Tween-20, pH 7.4, 82% ethylene glycol and 5 mg/mL polybrene). Polybrene (hexadimethrine bromide) at a final concentration of 5 mg/mL was added to Buffer C to quench the AT-III/UFH reaction. Residual activity of FIXa was assessed by following the initial rates of substrate cleavage for 60 minutes in a fluorescence reader set to 25° C. The final assay conditions for determination of residual activity were 0.2 nM FIXa variant, 0.83 mM Mesyl-D-CHG-Gly-Arg-AMC, 30% ethylene glycol and 5 mg/mL polybrene in 50 mM Tris, 100 mM NaCl, 10 mM CaCl₂), 0.01% Tween-20, pH 7.4. Data analyses were performed as described above for AT-III/LMWH inhibition assays.

As found with LMWH, AT-III/UFH inhibited less than 10-15% of the of the total protease activity for a number of FIXa variants at the highest tested concentrations of AT-III, thus representing an upper limit of detection for the assay under standard screening conditions. These variants with less than 10% maximal inhibition were therefore assigned a lower limit K_{0.5} value of 999 nM and in most cases are expected to have AT-III resistances much greater than the reported value. Several FIXa variants that were initially given a K_{0.5} value of 999 nM were retested at higher AT-III concentrations, expanding the sensitivity of the assay and providing clear levels of AT-III resistance. If these variants still maintained less than 10% maximal inhibition at the highest test AT-III concentrations (1000 nM to 5000 nM) a lower limit K_{0.5} value of 9999 nM was assigned, thus these variants are expected to have AT-III resistances much greater than the reported value.

Tables 30-31 provide the results of the assays that were performed using AT-III/UFH. Table 31 reflects data for additional FIXa variants and provide new overall averages calculated to include additional experimental replicates (n) for FIXa variants in Table 30. The results are presented both as the fitted K_{0.5} parameter and as a representation of the extent of AT-III resistance for each variant compared to the wild-type FIXa expressed as a ratio of their fitted K_{0.5} values (K_{0.5} variant/K_{0.5} wild-type). Several FIXa variants exhibited greater than 100 to 500-fold increased resistance to AT-III compared to wild-type FIXa. For example, FIXa-R318A/R403A, FIXa-R318A, FIXa-R318Y, FIXa-R338A/R403A FIXa-D203N/F205T/R318Y, FIXa-R318Y/R338E/R403E, FIXa-R318Y/R338E/R403E, FIXa-R318Y/R338E/E410N, R318Y/R338E/T343R/N346Y/R403E/E410N and FIXa-R318Y/R403E/E410N are among this group, which exhibited significant resistance to AT-III.

TABLE 30
Inhibition of FIXa variants by AT-III/UFH

| Mutation | Mutation | K_{0.5} | ±S.D. | | K_{0.5-mut}/ | |
|---|---|---|---|---|---|---|
| (Mature FIX Numbering) | (Chymotrypsin Numbering) | (nM) | (nM) | % CV | K_{0.5-wt} | n |
| BeneFIX ® Coagulation | BeneFIX ® Coagulation | 18 | 8 | 44% | 0.9 | 51 |
| FIX (T148A) | FIX (T[148]A) | | | | | |
| Plasma Purified FIXa | Plasma Purified FIXa | 30 | 4 | 14% | 1.6 | 5 |
| Catalyst Biosciences WT | Catalyst Biosciences WT | 19 | 7 | 34% | 1.0 | 15 |
| N157D | N[157]D | 17 | 4 | 23% | 0.9 | 2 |
| Y155F | Y[155]F | 13 | 0 | 1% | 0.7 | 2 |
| A103N/N105S/Y155F | A[103]N/N[105]S/Y[155]F | 11 | 6 | 49% | 0.6 | 2 |
| D104N/K106S/Y155F | D[104]N/K[106]S/Y[155]F | 6 | 2 | 33% | 0.3 | 2 |
| A103N/N105S | A[103]N/N[105]S | 20 | 3 | 14% | 1.0 | 2 |
| D104N/K106S | D[104]N/K[106]S | 20 | 2 | 9% | 1.0 | 2 |
| K106N/V108S | K[106]N/V[108]S | 24 | 0 | 1% | 1.2 | 2 |
| D85N | D[85]N | 17 | 3 | 15% | 0.9 | 4 |
| T148A | T[148]A | 21 | 8 | 39% | 1.1 | 10 |
| K5A | K[5]A | 22 | 3 | 15% | 1.2 | 2 |
| D64N | D[64]N | 18 | 0 | 1% | 0.9 | 2 |
| D64A | D[64]A | 16 | 2 | 12% | 0.8 | 2 |
| N167D | N[167]D | 12 | 2 | 14% | 0.6 | 2 |
| N167Q | N[167]Q | 12 | 1 | 8% | 0.6 | 2 |
| S61A | S[61]A | 19 | 3 | 18% | 1.0 | 2 |
| S53A | S[53]A | 27 | 4 | 16% | 1.4 | 2 |
| T159A | T[159]A | 33 | 7 | 23% | 1.7 | 2 |
| T169A | T[169]A | 17 | 6 | 36% | 0.9 | 2 |
| T172A | T[172]A | 16 | 3 | 21% | 0.8 | 2 |
| T179A | T[179]A | 24 | 2 | 7% | 1.2 | 2 |
| Y155H | Y[155]H | 25 | 4 | 15% | 1.3 | 2 |
| Y155Q | Y[155]Q | 23 | 0 | 1% | 1.2 | 2 |
| S158A | S[158]A | 20 | 1 | 5% | 1.0 | 2 |
| S158D | S[158]D | 15 | 2 | 16% | 0.8 | 2 |
| S158E | S[158]E | 14 | 1 | 10% | 0.7 | 2 |
| N157Q | N[157]Q | 16 | 2 | 11% | 0.8 | 2 |
| D203N/F205T | D39N/F41T | 271 | 51 | 19% | 14.0 | 5 |
| D85N/D203N/F205T | D[85]N/D39N/F41T | 587 | 65 | 11% | 30.3 | 2 |
| K228N | K63N | 29 | 13 | 46% | 1.5 | 6 |
| D85N/K228N | D[85]N/K63N | 34 | 3 | 7% | 1.7 | 2 |
| A103N/N105S/K228N | A[103]N/N[105]S/K63N | 46 | 17 | 36% | 2.4 | 2 |
| D104N/K106S/K228N | D[104]N/K[106]S/K63N | 41 | 21 | 52% | 2.1 | 2 |
| Y155F/K228N | Y[155]F/K63N | 15 | n.d. | n.d. | 0.8 | 1 |
| D104N/K106S/Y155F/K228N | D[104]N/K[106]S/Y[155]F/K63N | 49 | 5 | 9% | 2.5 | 2 |
| I251S | I86S | 28 | 8 | 28% | 1.4 | 4 |
| D85N/I251S | D[85]N/I86S | 19 | 6 | 30% | 1.0 | 2 |
| D85N/D104N/K106S/I251S | D[85]N/D[104]N/K[106]S/I86S | 28 | 11 | 41% | 1.4 | 2 |
| A103N/N105S/I251S | A[103]N/N[105]S/I86S | 42 | 14 | 33% | 2.2 | 3 |
| D104N/K106S/I251S | D[104]N/K[106]S/I86S | 32 | 5 | 16% | 1.6 | 2 |
| Y155F/I251S | Y[155]F/I86S | 18 | 3 | 19% | 0.9 | 2 |
| A262S | A95bS | 25 | 5 | 21% | 1.3 | 2 |
| K413N | K243N | 27 | 13 | 48% | 1.4 | 2 |
| E410N | E240N | 9 | 2 | 27% | 0.5 | 4 |
| E239N | E74N | 132 | 21 | 16% | 6.8 | 2 |
| T241N/H243S | T76N/H78S | 21 | 12 | 56% | 1.1 | 2 |
| K247N/N249S | K82N/N84S | 22 | 4 | 18% | 1.1 | 4 |
| Y155F/K247N/N249S | Y[155]F/K82N/N84S | 13 | 3 | 24% | 0.7 | 4 |
| A103N/N105S/K247N/N249S | A[103]N/N[105]S/K82N/N84S | 53 | 29 | 55% | 2.7 | 4 |
| D104N/K106S/K247N/N249S | D[104]N/K[106]S/K82N/N84S | 19 | 2 | 9% | 1.0 | 2 |
| D104N/K106S/Y155F/K247N/ | D[104]N/K[106]S/Y[155]F/K82N/ | 27 | 2 | 9% | 1.4 | 2 |
| N249S | N84S | | | | | |
| L321N | L153N | 25 | 6 | 25% | 1.3 | 2 |
| F314N/H315S | F145N/H147S | 104 | 27 | 26% | 5.4 | 4 |
| S319N/L321S | S151N/L153S | 65 | 11 | 17% | 3.4 | 2 |
| N260S | N95S | 312 | 283 | 91% | 16.1 | 13 |
| D104N/K106S/N260S | D[104]N/K[106]S/N95S | 228 | 82 | 36% | 11.8 | 2 |
| Y155F/N260S | Y[155]F/N95S | 77 | 16 | 21% | 4.0 | 2 |
| D104N/K106S/Y155F/N260S | D[104]N/K[106]S/Y[155]F/N95S | 292 | 37 | 13% | 15.1 | 2 |
| Y284N | Y117N | 41 | 25 | 63% | 2.1 | 5 |
| R318N/A320S | R150N/A152S | 999 | 0 | 0% | 51.7 | 2 |
| R318A | R150A | 4145 | 1297 | 31% | 214.3 | 2 |
| R318E | R150E | 10000 | 0 | 0% | 517.0 | 2 |
| R318Y | R150Y | 1976 | 430 | 22% | 102.2 | 2 |
| R312Q | R143Q | 33 | 9 | 26% | 1.7 | 2 |
| R312A | R143A | 31 | 0 | 1% | 1.6 | 2 |
| R312Y | R143Y | 2499 | 350 | 14% | 129.2 | 2 |
| R312L | R143L | 17 | 1 | 5% | 0.9 | 2 |
| V202M | V38M | 14 | 2 | 14% | 0.7 | 2 |
| V202Y | V38Y | 18 | 3 | 14% | 0.9 | 2 |
| D203M | D39M | 11 | 0 | 1% | 0.6 | 2 |
| D203Y | D39Y | 16 | 3 | 21% | 0.8 | 2 |
| A204M | A40M | 29 | 3 | 9% | 1.5 | 2 |
| A204Y | A40Y | 24 | 1 | 3% | 1.2 | 2 |
| K400A/R403A | K230A/R233A | 999 | 0 | 0% | 51.7 | 2 |
| K400E/R403E | K230E/R233E | 999 | 0 | 0% | 51.7 | 2 |
| R403A | R233A | 190 | 34 | 18% | 9.8 | 4 |
| R403E | R233E | 731 | 14 | 2% | 37.8 | 2 |
| K400A | K230A | 114 | 3 | 3% | 5.9 | 2 |
| K400E | K230E | 301 | 27 | 9% | 15.6 | 2 |
| K293E | K126E | 187 | 25 | 13% | 9.7 | 2 |
| K293A | K126A | 82 | 1 | 1% | 4.2 | 2 |
| R333A | R165A | 235 | 54 | 23% | 12.1 | 2 |
| R333E | R165E | 999 | 0 | 0% | 51.7 | 2 |
| R338A | R170A | 33 | 3 | 10% | 1.7 | 2 |
| R338E | R170E | 222 | 124 | 56% | 11.5 | 8 |
| R338A/R403A | R170A/R233A | 328 | 106 | 32% | 17.0 | 6 |
| R338E/R403E | R170E/R233E | 6000 | 1089 | 18% | 310.2 | 2 |
| K293A/R403A | K126A/R233A | 999 | 0 | 0% | 51.7 | 2 |
| K293E/R403E | K126E/R233E | 999 | 0 | 0% | 51.7 | 2 |
| K293A/R338A/R403A | K126A/R170A/R233A | 999 | 0 | 0% | 51.7 | 2 |
| K293E/R338E/R403E | K126E/R170E/R233E | 999 | 0 | 0% | 51.7 | 2 |
| R318A/R403A | R150A/R233A | 999 | 0 | 0% | 51.7 | 2 |
| R318E/R403E | R150E/R233E | 999 | 0 | 0% | 51.7 | 2 |
| R318Y/E410N | R150Y/E240N | 607 | 164 | 27% | 31.4 | 4 |
| R338E/E410N | R170E/E240N | 92 | 14 | 15% | 4.7 | 4 |
| R338E/R403E/E410N | R170E/R233E/E240N | 2351 | 168 | 7% | 121.5 | 2 |
| R318Y/R338E/R403E | R150Y/R170E/R233E | 10000 | 0 | 0% | 517.0 | 7 |
| D203N/F205T/K228N | D39N/F41T/K63N | 822 | 69 | 8% | 42.5 | 2 |
| D203N/F205T/E410N | D39N/F41T/E240N | 377 | 20 | 5% | 19.5 | 2 |
| D203N/F205T/R338E | D39N/F41T/R170E | 1170 | 180 | 15% | 60.5 | 2 |
| D203N/F205T/R338A | D39N/F41T/R170A | 423 | 61 | 14% | 21.9 | 2 |
| D203N/F205T/R318Y | D39N/F41T/R150Y | 7226 | 133 | 2% | 373.6 | 2 |
| D203N/F205T/R338E/R403E | D39N/F41T/R170E/R233E | 1520 | 162 | 11% | 78.6 | 2 |
| K228N/E410N | K63N/E240N | 36 | 7 | 20% | 1.9 | 2 |
| K228N/R338E | K63N/R170E | 108 | 8 | 7% | 5.6 | 2 |
| K228N/R338A | K63N/R170A | 51 | 7 | 14% | 2.7 | 2 |
| K228N/R318Y | K63N/R150Y | 3414 | 73 | 2% | 176.5 | 2 |
| K228N/R338E/R403E | K63N/R170E/R233E | 1679 | 239 | 14% | 86.8 | 2 |
| R403E/E410N | R233E/E240N | 279 | 26 | 9% | 14.4 | 2 |
| R318Y/R338E/E410N | R150Y/R170E/E240N | 3458 | 1033 | 30% | 178.8 | 5 |
| D104N/K106S/R318Y/R338E/ | D[104]N/K[106]S/R150Y/R170E/ | 6328 | 4241 | 67% | 327.2 | 4 |
| E410N | E240N | | | | | |
| Y155F/R318Y/R338E/E410N | Y[155]F/R150Y/R170E/E240N | 1098 | 1095 | 100% | 56.8 | 7 |
| K228N/R318Y/E410N | K63N/R150Y/E240N | 475 | 83 | 17% | 24.6 | 2 |
| R318Y/R403E/E410N | R150Y/R233E/E240N | 7072 | 1387 | 20% | 365.6 | 2 |
| R318Y/R338E/R403E/E410N | R150Y/R170E/R233E/E240N | 5881 | 4757 | 81% | 304.1 | 4 |
| A103N/N105S/R318Y/R338E/ | A[103]N/N[105]S/R150Y/R170E/ | 9193 | 1037 | 11% | 475.3 | 4 |
| R403E/E410N | R233E/E240N | | | | | |
| D104N/K106S/R318Y/R338E/ | D[104]N/K[106]S/R150Y/R170E/ | 10000 | 0 | 0% | 517.0 | 2 |
| R403E/E410N | R233E/E240N | | | | | |
| Y155F/R318Y/R338E/R403E/ | Y[155]F/R150Y/R170E/R233E/ | 10000 | 0 | 0% | 517.0 | 2 |
| E410N | E240N | | | | | |
| A103N/N105S/Y155F/R318Y/ | A[103]N/N[105]S/Y[155]F/R150Y/ | 10000 | 0 | 0% | 517.0 | 2 |
| R338E/R403E/E410N | R170E/R233E/E240N | | | | | |
| D104N/K106S/Y155F/R318Y/ | D[104]N/K[106]S/Y[155]F/R150Y/ | 10000 | 0 | 0% | 517.0 | 2 |
| R338E/R403E/E410N | R170E/R233E/E240N | | | | | |
| D203N/F205T/R318Y/E410N | D39N/F41T/R150Y/E240N | 1280 | 220 | 17% | 66.2 | 2 |
| R333S | R165S | 720 | 67 | 9% | 37.2 | 2 |
| R338L | R170L | 121 | 6 | 5% | 6.3 | 2 |
| K316N | K148N | 56 | 2 | 4% | 2.9 | 2 |
| K316A | K148A | 63 | 15 | 24% | 3.2 | 2 |
| K316E | K148E | 183 | 2 | 1% | 9.5 | 2 |
| K316S | K148S | 77 | 15 | 19% | 4.0 | 2 |
| K316M | K148M | 9 | 2 | 24% | 0.5 | 2 |
| E239S | E74S | 101 | 12 | 12% | 5.2 | 2 |
| E239A | E74A | 30 | 14 | 47% | 1.6 | 3 |
| E239R | E74R | 65 | 17 | 26% | 3.3 | 2 |
| E239K | E74K | 19 | 4 | 22% | 1.0 | 2 |
| H257F | H92F | 12 | 1 | 11% | 0.6 | 2 |
| H257Y | H92Y | 20 | 2 | 12% | 1.0 | 2 |
| H257E | H92E | 25 | 12 | 48% | 1.3 | 3 |
| H257S | H92S | 23 | 21 | 89% | 1.2 | 3 |
| T412A | T242A | 25 | 3 | 14% | 1.3 | 4 |
| T412V | T242V | 23 | 4 | 16% | 1.2 | 4 |
| E410N/T412A | E240N/T242A | 10 | 1 | 7% | 0.5 | 2 |
| E410N/T412V | E240N/T242V | 11 | 3 | 24% | 0.6 | 2 |
| E410Q | E240Q | 24 | 14 | 60% | 1.2 | 4 |
| E410S | E240S | 26 | 16 | 63% | 1.3 | 7 |
| E410A | E240A | 42 | 24 | 58% | 2.2 | 6 |
| E410D | E240D | 41 | 2 | 5% | 2.1 | 2 |
| N346D | N178D | 222 | 176 | 79% | 11.5 | 5 |
| Y155F/N346D | Y[155]F/N178D | 223 | 102 | 46% | 11.5 | 2 |
| N346Y | N178Y | 36 | 2 | 7% | 1.9 | 4 |
| Y345A | Y177A | 96 | 87 | 90% | 5.0 | 13 |
| Y345T | Y177T | 16 | 0 | 0% | 0.8 | 2 |
| T343R | T175R | 7 | 1 | 10% | 0.4 | 2 |
| T343E | T175E | 55 | 8 | 15% | 2.8 | 2 |
| T343Q | T175Q | 13 | 3 | 25% | 0.7 | 2 |
| F342I | F174I | 98 | 10 | 11% | 5.1 | 2 |
| T343R/Y345T | T175R/Y177T | 6 | 0 | 4% | 0.3 | 2 |
| R318Y/R338E | R150Y/R170E | 397 | 50 | 12% | 20.5 | 2 |
| Y259F/K265T/Y345T | Y94F/K98T/Y177T | 6 | 0 | 2% | 0.3 | 2 |
| K228N/I251S | K63N/I86S | 73 | 16 | 22% | 3.8 | 2 |
| K228N/R318Y/R338E/R403E/ | K63N/R150Y/R170E/R233E/E240N | 10000 | 0 | 0% | 517.0 | 2 |
| E410N | | | | | | |
| Y155F/K228N/R318Y/R338E/ | Y[155]F/K63N/R150Y/R170E/ | 10000 | 0 | 0% | 517.0 | 2 |
| R403E/E410N | R233E/E240N | | | | | |
| D85N/K228N/R318Y/R338E/ | D[85]N/K63N/R150Y/R170E/ | 10000 | 0 | 0% | 517.0 | 2 |
| R403E/E410N | R233E/E240N | | | | | |
| I251S/R318Y/R338E/R403E/ | I86S/R150Y/R170E/R233E/E240N | 10000 | 0 | 0% | 517.0 | 2 |
| E410N | | | | | | |
| D104N/K106S/I251S/R318Y/ | D[104]N/K[106]S/I86S/R150Y/ | 10000 | 0 | 0% | 517.0 | 3 |
| R338E/R403E/E410N | R170E/R233E/E240N | | | | | |
| Y155F/I251S/R318Y/R338E/ | Y[155]F/I86S/R150Y/R170E/R233E/ | 10000 | 0 | 0% | 517.0 | 2 |
| R403E/E410N | E240N | | | | | |
| I251S/R318Y/R338E/E410N | I86S/R150Y/R170E/E240N | 5855 | 3889 | 66% | 302.7 | 7 |
| D104N/K106S/I251S/R318Y/ | D[104]N/K[106]S/I86S/R150Y/ | 8985 | 1436 | 16% | 464.5 | 2 |
| R338E/E410N | R170E/E240N | | | | | |
| F314N/K316S | F145N/K148S | 1221 | 505 | 41% | 63.1 | 4 |
| K247N/N249S/R318Y/R338E/ | K82N/N84S/R150Y/R170E/R233E/ | 8076 | 2967 | 37% | 417.6 | 9 |
| R403E/E410N | E240N | | | | | |
| Y155F/K247N/N249S/R318Y/ | Y[155]F/K82N/N84S/R150Y/R170E/ | 10000 | 0 | 0% | 517.0 | 3 |
| R338E/R403E/E410N | R233E/E240N | | | | | |
| A103N/N105S/K247N/N249S/ | A[103]N/N[105]S/K82N/N84S/ | 2497 | 772 | 31% | 129.1 | 4 |
| R318Y/R338E/R403E/E410N | R150Y/R170E/R233E/E240N | | | | | |
| D104N/K106S/K247N/N249S/ | D[104]N/K[106]S/K82N/N84S/ | 10000 | 0 | 0% | 517.0 | 2 |
| R318Y/R338E/R403E/E410N | R150Y/R170E/R233E/E240N | | | | | |
| K247N/N249S/R318Y/R338E/ | K82N/N84S/R150Y/R170E/E240N | 1514 | 631 | 42% | 78.3 | 3 |
| E410N | | | | | | |
| Y155F/K247N/N249S/R318Y/ | Y[155]F/K82N/N84S/R150Y/R170E/ | 3875 | 846 | 22% | 200.4 | 2 |
| R338E/E410N | E240N | | | | | |
| R318Y/R338E/R403E/E410S | R150Y/R170E/R233E/E240S | 10000 | 0 | 0% | 517.0 | 2 |
| R318Y/R338E/E410S | R150Y/R170E/E240S | 5402 | 2785 | 52% | 279.3 | 5 |
| K228N/K247N/N249S | K63N/K82N/N84S | 85 | 19 | 22% | 4.4 | 2 |
| D104N/K106S/Y155F/K228N/ | D[104]N/K[106]S/Y[155]F/K63N/ | 32 | 12 | 37% | 1.6 | 4 |
| K247N/N249S | K82N/N84S | | | | | |
| D104N/K106S/K228N/K247N/ | D[104]N/K[106]S/K63N/K82N/ | 41 | 18 | 45% | 2.1 | 10 |
| N249S | N84S | | | | | |
| Y155F/K228N/K247N/N249S | Y[155]F/K63N/K82N/N84S | 27 | 6 | 22% | 1.4 | 2 |
| K228N/K247N/N249S/R318Y/ | K63N/K82N/N84S/R150Y/R170E/ | 10000 | 0 | 0% | 517.0 | 2 |
| R338E/R403E/E410N | R233E/E240N | | | | | |
| R318Y/R338E/R403E/E410N/ | R150Y/R170E/R233E/E240N/ | 10000 | 0 | 0% | 517.0 | 2 |
| T412V | T242V | | | | | |
| R318Y/R338E/R403E/E410N/ | R150Y/R170E/R233E/E240N/ | 10000 | 0 | 0% | 517.0 | 2 |
| T412A | T242A | | | | | |
| R318Y/R338E/R403E/T412A | R150Y/R170E/R233E/T242A | 10000 | 0 | 0% | 517.0 | 2 |
| R318Y/R338E/T412A | R150Y/R170E/T242A | 7661 | 3243 | 42% | 396.1 | 9 |
| R318Y/R338E/E410N/T412V | R150Y/R170E/E240N/T242V | 10000 | 0 | 0% | 517.0 | 2 |
| N260S/R318Y/R338E/R403E/ | N95S/R150Y/R170E/R233E/E240N | 10000 | 0 | 0% | 517.0 | 2 |
| E410N | | | | | | |
| D104N/K106S/N260S/R318Y/ | D[104]N/K[106]S/N95S/R150Y/ | 10000 | 0 | 0% | 517.0 | 3 |
| R338E/R403E/E410N | R170E/R233E/E240N | | | | | |
| Y155F/N260S/R318Y/R338E/ | Y[155]F/N95S/R150Y/R170E/ | 9696 | 527 | 5% | 501.3 | 3 |
| R403E/E410N | R233E/E240N | | | | | |
| R318Y/R338E/N346D/R403E/ | R150Y/R170E/N178D/R233E/ | 10000 | 0 | 0% | 517.0 | 2 |
| E410N | E240N | | | | | |
| Y155F/R318Y/R338E/N346D/ | Y[155]F/R150Y/R170E/N178D/ | 10000 | 0 | 0% | 517.0 | 2 |
| R403E/E410N | R233E/E240N | | | | | |
| K247N/N249S/N260S | K82N/N84S/N95S | 157 | 38 | 24% | 8.1 | 3 |
| Y155F/K247N/N249S/N260S | Y[155]F/K82N/N84S/N95S | 152 | 39 | 26% | 7.9 | 3 |
| D[104]N/K[106]S/K247N/N249S/ | D[104]N/K[106]S/K82N/N84S/ | 1262 | 40 | 3% | 65.3 | 2 |
| N260S | N95S | | | | | |
| D[104]N/K[106]S/Y[155]F/K247N/ | D[104]N/K[106]S/Y[155]F/K82N/ | 692 | 84 | 12% | 35.8 | 2 |
| N249S/N260S | N84S/N95S | | | | | |
| K247N/N249S/N260S/R318Y/ | K82N/N84S/N95S/R150Y/R170E/ | 5560 | 3872 | 70% | 287.5 | 3 |
| R338E/R403E/E410N | R233E/E240N | | | | | |
| Y155F/N260S/N346D | Y[155]F/N95S/N178D | 1382 | 477 | 35% | 71.4 | 2 |
| R318Y/R338E/T343R/R403E/ | R150Y/R170E/T175R/R233E/ | 10000 | 0 | 0% | 517.0 | 2 |
| E410N | E240N | | | | | |
| R338E/T343R | R170E/T175R | 16 | 6 | 38% | 0.8 | 2 |

* A K_{0.5} value of 999 nM indicates the lower limit value for those variants with less than 10% inhibition under the conditions of the standard assay (35 nM-0 nM AT-III).
* Variants with >50% of WT k_{cat}/K_{M} (see Example 4, Table 23) and initially given a K_{0.5} value of 999 nM were retested at higher AT-III concentrations, expanding in the sensitivity of the assay.
* A K_{0.5} value of 9999 nM indicates the lower limit value for those variants with less than 10% inhibition under the conditions of the expanded sensitivity assay (1000 nM-0 nM AT-III and 5000-0 nM AT-III).

TABLE 31
Inhibition of FIXa variants by AT-III/UFH

| Mutation | Mutation | K_{0.5} | ±S.D. | | K_{0.5-mut}/ | |
|---|---|---|---|---|---|---|
| (Mature FIX Numbering) | (Chymotrypsin Numbering) | (nM) | (nM) | % CV | K_{0.5-wt} | n |
| BeneFIX ® Coagulation | BeneFIX ® Coagulation | 17 | 8 | 47% | 0.9 | 55 |
| FIX (T148A) | FIX (T[148]A) | | | | | |
| Plasma Purified FIXa | Plasma Purified FIXa | 30 | 4 | 14% | 1.6 | 5 |
| Catalyst Biosciences WT | Catalyst Biosciences WT | 19 | 7 | 34% | 1.0 | 15 |
| N157D | N[157]D | 17 | 4 | 23% | 0.9 | 2 |
| Y155F | Y[155]F | 13 | 0 | 1% | 0.7 | 2 |
| A103N/N105S/Y155F | A[103]N/N[105]S/Y[155]F | 11 | 6 | 49% | 0.6 | 2 |
| D104N/K106S/Y155F | D[104]N/K[106]S/Y[155]F | 6 | 2 | 33% | 0.3 | 2 |
| A103N/N105S | A[103]N/N[105]S | 20 | 3 | 14% | 1.0 | 2 |
| D104N/K106S | D[104]N/K[106]S | 20 | 2 | 9% | 1.0 | 2 |
| K106N/V108S | K[106]N/V[108]S | 24 | 0 | 1% | 1.2 | 2 |
| D85N | D[85]N | 17 | 3 | 15% | 0.9 | 4 |
| T148A | T[148]A | 17 | 10 | 56% | 0.9 | 13 |
| K5A | K[5]A | 22 | 3 | 15% | 1.2 | 2 |
| D64N | D[64]N | 18 | 0 | 1% | 0.9 | 2 |
| D64A | D[64]A | 16 | 2 | 12% | 0.8 | 2 |
| N167D | N[167]D | 12 | 2 | 14% | 0.6 | 2 |
| N167Q | N[167]Q | 12 | 1 | 8% | 0.6 | 2 |
| S61A | S[61]A | 19 | 3 | 18% | 1.0 | 2 |
| S53A | S[53]A | 27 | 4 | 16% | 1.4 | 2 |
| T159A | T[159]A | 33 | 7 | 23% | 1.7 | 2 |
| T169A | T[169]A | 17 | 6 | 36% | 0.9 | 2 |
| T172A | T[172]A | 16 | 3 | 21% | 0.8 | 2 |
| T179A | T[179]A | 24 | 2 | 7% | 1.2 | 2 |
| Y155H | Y[155]H | 25 | 4 | 15% | 1.3 | 2 |
| Y155Q | Y[155]Q | 23 | 0 | 1% | 1.2 | 2 |
| S158A | S[158]A | 20 | 1 | 5% | 1.0 | 2 |
| S158D | S[158]D | 15 | 2 | 16% | 0.8 | 2 |
| S158E | S[158]E | 14 | 1 | 10% | 0.7 | 2 |
| N157Q | N[157]Q | 16 | 2 | 11% | 0.8 | 2 |
| D203N/F205T | D39N/F41T | 271 | 51 | 19% | 14.0 | 5 |
| D85N/D203N/F205T | D[85]N/D39N/F41T | 587 | 65 | 11% | 30.3 | 2 |
| K228N | K63N | 29 | 13 | 46% | 1.5 | 6 |
| D85N/K228N | D[85]N/K63N | 34 | 3 | 7% | 1.7 | 2 |
| A103N/N105S/K228N | A[103]N/N[105]S/K63N | 46 | 17 | 36% | 2.4 | 2 |
| D104N/K106S/K228N | D[104]N/K[106]S/K63N | 41 | 21 | 52% | 2.1 | 2 |
| Y155F/K228N | Y[155]F/K63N | 15 | n.d. | n.d. | 0.8 | 1 |
| D104N/K106S/Y155F/K228N | D[104]N/K[106]S/Y[155]F/K63N | 49 | 5 | 9% | 2.5 | 2 |
| I251S | I86S | 28 | 8 | 28% | 1.4 | 4 |
| D85N/I251S | D[85]N/I86S | 19 | 6 | 30% | 1.0 | 2 |
| D85N/D104N/K106S/I251S | D[85]N/D[104]N/K[106]S/I86S | 28 | 11 | 41% | 1.4 | 2 |
| A103N/N105S/I251S | A[103]N/N[105]S/I86S | 42 | 14 | 33% | 2.2 | 3 |
| D104N/K106S/I251S | D[104]N/K[106]S/I86S | 32 | 5 | 16% | 1.6 | 2 |
| Y155F/I251S | Y[155]F/I86S | 18 | 3 | 19% | 0.9 | 2 |
| A262S | A95bS | 25 | 5 | 21% | 1.3 | 2 |
| K413N | K243N | 27 | 13 | 48% | 1.4 | 2 |
| E410N | E240N | 8 | 2 | 25% | 0.4 | 6 |
| E239N | E74N | 132 | 21 | 16% | 6.8 | 2 |
| T241N/H243S | T76N/H78S | 21 | 12 | 56% | 1.1 | 2 |
| K247N/N249S | K82N/N84S | 22 | 4 | 18% | 1.1 | 4 |
| Y155F/K247N/N249S | Y[155]F/K82N/N84S | 13 | 3 | 24% | 0.7 | 4 |
| A103N/N105S/K247N/N249S | A[103]N/N[105]S/K82N/N84S | 53 | 29 | 55% | 2.7 | 4 |
| D104N/K106S/K247N/N249S | D[104]N/K[106]S/K82N/N84S | 19 | 2 | 9% | 1.0 | 2 |
| D104N/K106S/Y155F/K247N/N249S | D[104]N/K[106]S/Y[155]F/K82N/ | 27 | 2 | 9% | 1.4 | 2 |
| | N84S | | | | | |
| L321N | L153N | 25 | 6 | 25% | 1.3 | 2 |
| F314N/H315S | F145N/H147S | 104 | 27 | 26% | 5.4 | 4 |
| S319N/L321S | S151N/L153S | 65 | 11 | 17% | 3.4 | 2 |
| N260S | N95S | 312 | 283 | 91% | 16.1 | 13 |
| D104N/K106S/N260S | D[104]N/K[106]S/N95S | 228 | 82 | 36% | 11.8 | 2 |
| Y155F/N260S | Y[155]F/N95S | 77 | 16 | 21% | 4.0 | 2 |
| D104N/K106S/Y155F/N260S | D[104]N/K[106]S/Y[155]F/N95S | 292 | 37 | 13% | 15.1 | 2 |
| Y284N | Y117N | 41 | 25 | 63% | 2.1 | 5 |
| R318N/A320S | R150N/A152S | 999 | 0 | 0% | 51.7 | 2 |
| R318A | R150A | 4145 | 1297 | 31% | 214.3 | 2 |
| R318E | R150E | 9999 | 0 | 0% | 517.0 | 2 |
| R318Y | R150Y | 1976 | 430 | 22% | 102.2 | 2 |
| R312Q | R143Q | 33 | 9 | 26% | 1.7 | 2 |
| R312A | R143A | 31 | 0 | 1% | 1.6 | 2 |
| R312Y | R143Y | 2499 | 350 | 14% | 129.2 | 2 |
| R312L | R143L | 17 | 1 | 5% | 0.9 | 2 |
| V202M | V38M | 14 | 2 | 14% | 0.7 | 2 |
| V202Y | V38Y | 18 | 3 | 14% | 0.9 | 2 |
| D203M | D39M | 11 | 0 | 1% | 0.6 | 2 |
| D203Y | D39Y | 16 | 3 | 21% | 0.8 | 2 |
| A204M | A40M | 29 | 3 | 9% | 1.5 | 2 |
| A204Y | A40Y | 24 | 1 | 3% | 1.2 | 2 |
| K400A/R403A | K230A/R233A | 999 | 0 | 0% | 51.7 | 2 |
| K400E/R403E | K230E/R233E | 999 | 0 | 0% | 51.7 | 2 |
| R403A | R233A | 190 | 34 | 18% | 9.8 | 4 |
| R403E | R233E | 731 | 14 | 2% | 37.8 | 2 |
| K400A | K230A | 114 | 3 | 3% | 5.9 | 2 |
| K400E | K230E | 301 | 27 | 9% | 15.6 | 2 |
| K293E | K126E | 187 | 25 | 13% | 9.7 | 2 |
| K293A | K126A | 82 | 1 | 1% | 4.2 | 2 |
| R333A | R165A | 235 | 54 | 23% | 12.1 | 2 |
| R333E | R165E | 999 | 0 | 0% | 51.7 | 2 |
| R338A | R170A | 33 | 3 | 10% | 1.7 | 2 |
| R338E | R170E | 222 | 124 | 56% | 11.5 | 8 |
| R338A/R403A | R170A/R233A | 328 | 106 | 32% | 17.0 | 6 |
| R338E/R403E | R170E/R233E | 6000 | 1089 | 18% | 310.2 | 2 |
| K293A/R403A | K126A/R233A | 999 | 0 | 0% | 51.7 | 2 |
| K293E/R403E | K126E/R233E | 999 | 0 | 0% | 51.7 | 2 |
| K293A/R338A/R403A | K126A/R170A/R233A | 999 | 0 | 0% | 51.7 | 2 |
| K293E/R338E/R403E | K126E/R170E/R233E | 999 | 0 | 0% | 51.7 | 2 |
| R318A/R403A | R150A/R233A | 999 | 0 | 0% | 51.7 | 2 |
| R318E/R403E | R150E/R233E | 999 | 0 | 0% | 51.7 | 2 |
| R318Y/E410N | R150Y/E240N | 607 | 164 | 27% | 31.4 | 4 |
| R338E/E410N | R170E/E240N | 92 | 14 | 15% | 4.7 | 4 |
| R338E/R403E/E410N | R170E/R233E/E240N | 2351 | 168 | 7% | 121.5 | 2 |
| R318Y/R338E/R403E | R150Y/R170E/R233E | 10000 | 0 | 0% | 517.0 | 7 |
| D203N/F205T/K228N | D39N/F41T/K63N | 822 | 69 | 8% | 42.5 | 2 |
| D203N/F205T/E410N | D39N/F41T/E240N | 377 | 20 | 5% | 19.5 | 2 |
| D203N/F205T/R338E | D39N/F41T/R170E | 1170 | 180 | 15% | 60.5 | 2 |
| D203N/F205T/R338A | D39N/F41T/R170A | 423 | 61 | 14% | 21.9 | 2 |
| D203N/F205T/R318Y | D39N/F41T/R150Y | 7226 | 133 | 2% | 373.6 | 2 |
| D203N/F205T/R338E/R403E | D39N/F41T/R170E/R233E | 1520 | 162 | 11% | 78.6 | 2 |
| K228N/E410N | K63N/E240N | 36 | 7 | 20% | 1.9 | 2 |
| K228N/R338E | K63N/R170E | 108 | 8 | 7% | 5.6 | 2 |
| K228N/R338A | K63N/R170A | 51 | 7 | 14% | 2.7 | 2 |
| K228N/R318Y | K63N/R150Y | 3414 | 73 | 2% | 176.5 | 2 |
| K228N/R338E/R403E | K63N/R170E/R233E | 1679 | 239 | 14% | 86.8 | 2 |
| R403E/E410N | R233E/E240N | 279 | 26 | 9% | 14.4 | 2 |
| R318Y/R338E/E410N | R150Y/R170E/E240N | 3458 | 1033 | 30% | 178.8 | 5 |
| D104N/K106S/R318Y/R338E/E410N | D[104]N/K[106]S/R150Y/R170E/ | 6328 | 4241 | 67% | 327.2 | 4 |
| | E240N | | | | | |
| Y155F/R318Y/R338E/E410N | Y[155]F/R150Y/R170E/E240N | 1098 | 1095 | 100% | 56.8 | 7 |
| K228N/R318Y/E410N | K63N/R150Y/E240N | 475 | 83 | 17% | 24.6 | 2 |
| R318Y/R403E/E410N | R150Y/R233E/E240N | 7072 | 1387 | 20% | 365.6 | 2 |
| R318Y/R338E/R403E/E410N | R150Y/R170E/R233E/E240N | 5881 | 4757 | 81% | 304.1 | 4 |
| A103N/N105S/R318Y/R338E/R403E/ | A[103]N/N[105]S/R150Y/R170E/ | 9193 | 1037 | 11% | 475.3 | 4 |
| E410N | R233E/E240N | | | | | |
| D104N/K106S/R318Y/R338E/R403E/ | D[104]N/K[106]S/R150Y/R170E/ | 10000 | 0 | 0% | 517.0 | 2 |
| E410N | R233E/E240N | | | | | |
| Y155F/R318Y/R338E/R403E/E410N | Y[155]F/R150Y/R170E/R233E/ | 10000 | 0 | 0% | 517.0 | 2 |
| | E240N | | | | | |
| A103N/N105S/Y155F/R318Y/R338E/ | A[103]N/N[105]S/Y[155]F/R150Y/ | 10000 | 0 | 0% | 517.0 | 2 |
| R403E/E410N | R170E/R233E/E240N | | | | | |
| D104N/K106S/Y155F/R318Y/R338E/ | D[104]N/K[106]S/Y[155]F/R150Y/ | 10000 | 0 | 0% | 517.0 | 2 |
| R403E/E410N | R170E/R233E/E240N | | | | | |
| D203N/F205T/R318Y/E410N | D39N/F41T/R150Y/E240N | 1280 | 220 | 17% | 66.2 | 2 |
| R333S | R165S | 720 | 67 | 9% | 37.2 | 2 |
| R338L | R170L | 121 | 6 | 5% | 6.3 | 2 |
| K316N | K148N | 56 | 2 | 4% | 2.9 | 2 |
| K316A | K148A | 63 | 15 | 24% | 3.2 | 2 |
| K316E | K148E | 183 | 2 | 1% | 9.5 | 2 |
| K316S | K148S | 77 | 15 | 19% | 4.0 | 2 |
| K316M | K148M | 9 | 2 | 24% | 0.5 | 2 |
| E239S | E74S | 101 | 12 | 12% | 5.2 | 2 |
| E239A | E74A | 30 | 14 | 47% | 1.6 | 3 |
| E239R | E74R | 65 | 17 | 26% | 3.3 | 2 |
| E239K | E74K | 19 | 4 | 22% | 1.0 | 2 |
| H257F | H92F | 12 | 1 | 11% | 0.6 | 2 |
| H257Y | H92Y | 20 | 2 | 12% | 1.0 | 2 |
| H257E | H92E | 25 | 12 | 48% | 1.3 | 3 |
| H257S | H92S | 23 | 21 | 89% | 1.2 | 3 |
| T412A | T242A | 25 | 3 | 14% | 1.3 | 4 |
| T412V | T242V | 23 | 4 | 16% | 1.2 | 4 |
| E410N/T412A | E240N/T242A | 10 | 1 | 7% | 0.5 | 2 |
| E410N/T412V | E240N/T242V | 11 | 3 | 24% | 0.6 | 2 |
| E410Q | E240Q | 24 | 14 | 60% | 1.2 | 4 |
| E410S | E240S | 26 | 16 | 63% | 1.3 | 7 |
| E410A | E240A | 42 | 24 | 58% | 2.2 | 6 |
| E410D | E240D | 41 | 2 | 5% | 2.1 | 2 |
| N346D | N178D | 222 | 176 | 79% | 11.5 | 5 |
| Y155F/N346D | Y[155]F/N178D | 223 | 102 | 46% | 11.5 | 2 |
| N346Y | N178Y | 36 | 2 | 7% | 1.9 | 4 |
| Y345A | Y177A | 96 | 87 | 90% | 5.0 | 13 |
| Y345T | Y177T | 16 | 0 | 0% | 0.8 | 2 |
| T343R | T175R | 7 | 1 | 10% | 0.4 | 2 |
| T343E | T175E | 55 | 8 | 15% | 2.8 | 2 |
| T343Q | T175Q | 13 | 3 | 25% | 0.7 | 2 |
| F342I | F174I | 98 | 10 | 11% | 5.1 | 2 |
| T343R/Y345T | T175R/Y177T | 6 | 0 | 4% | 0.3 | 2 |
| R318Y/R338E | R150Y/R170E | 397 | 50 | 12% | 20.5 | 2 |
| Y259F/K265T/Y345T | Y94F/K98T/Y177T | 6 | 0 | 2% | 0.3 | 2 |
| K228N/I251S | K63N/I86S | 73 | 16 | 22% | 3.8 | 2 |
| K228N/R318Y/R338E/R403E/E410N | K63N/R150Y/R170E/R233E/ | 10000 | 0 | 0% | 517.0 | 2 |
| | E240N | | | | | |
| Y155F/K228N/R318Y/R338E/R403E/ | Y[155]F/K63N/R150Y/R170E/ | 10000 | 0 | 0% | 517.0 | 2 |
| E410N | R233E/E240N | | | | | |
| D85N/K228N/R318Y/R338E/R403E/ | D[85]N/K63N/R150Y/R170E/ | 10000 | 0 | 0% | 517.0 | 2 |
| E410N | R233E/E240N | | | | | |
| I251S/R318Y/R338E/R403E/E410N | I86S/R150Y/R170E/R233E/E240N | 10000 | 0 | 0% | 517.0 | 2 |
| D104N/K106S/I251S/R318Y/R338E/ | D[104]N/K[106]S/I86S/R150Y/ | 10000 | 0 | 0% | 517.0 | 3 |
| R403E/E410N | R170E/R233E/E240N | | | | | |
| Y155F/I251S/R318Y/R338E/R403E/ | D[104]N/K[106]S/I86S/R150Y/ | 10000 | 0 | 0% | 517.0 | 2 |
| E410N | R170E/R233E/E240N | | | | | |
| I251S/R318Y/R338E/E410N | I86S/R150Y/R170E/E240N | 5855 | 3889 | 66% | 302.7 | 7 |
| D104N/K106S/I251S/R318Y/R338E/ | D[104]N/K[106]S/I86S/R150Y/ | 8985 | 1436 | 16% | 464.5 | 2 |
| E410N | R170E/E240N | | | | | |
| F314N/K316S | F145N/K148S | 1221 | 505 | 41% | 63.1 | 4 |
| K247N/N249S/R318Y/R338E/R403E/ | K82N/N84S/R150Y/R170E/R233E/ | 8076 | 2967 | 37% | 417.6 | 9 |
| E410N | E240N | | | | | |
| Y155F/K247N/N249S/R318Y/R338E/ | Y[155]F/K82N/N84S/R150Y/ | 10000 | 0 | 0% | 517.0 | 3 |
| R403E/E410N | R170E/R233E/E240N | | | | | |
| A103N/N105S/K247N/N249S/R318Y/ | A[103]N/N[105]S/K82N/N84S/ | 2497 | 772 | 31% | 129.1 | 4 |
| R338E/R403E/E410N | R150Y/R170E/R233E/E240N | | | | | |
| D104N/K106S/K247N/N249S/R318Y/ | D[104]N/K[106]S/K82N/N84S/ | 10000 | 0 | 0% | 517.0 | 2 |
| R338E/R403E/E410N | R150Y/R170E/R233E/E240N | | | | | |
| K247N/N249S/R318Y/R338E/E410N | K82N/N84S/R150Y/R170E/E240N | 1514 | 631 | 42% | 78.3 | 3 |
| Y155F/K247N/N249S/R318Y/R338E/ | Y[155]F/K82N/N84S/R150Y/ | 3875 | 846 | 22% | 200.4 | 2 |
| E410N | R170E/E240N | | | | | |
| R318Y/R338E/R403E/E410S | R150Y/R170E/R233E/E240S | 10000 | 0 | 0% | 517.0 | 2 |
| R318Y/R338E/E410S | R150Y/R170E/E240S | 5402 | 2785 | 52% | 279.3 | 5 |
| K228N/K247N/N249S | K63N/K82N/N84S | 85 | 19 | 22% | 4.4 | 2 |
| D104N/K106S/Y155F/K228N/K247N/ | D[104]N/K[106]S/Y[155]F/K63N/ | 32 | 12 | 37% | 1.6 | 4 |
| N249S | K82N/N84S | | | | | |
| D104N/K106S/K228N/K247N/ | D[104]N/K[106]S/K63N/K82N/ | 41 | 18 | 45% | 2.1 | 10 |
| N249S | N84S | | | | | |
| Y155F/K228N/K247N/N249S | Y[155]F/K63N/K82N/N84S | 27 | 6 | 22% | 1.4 | 2 |
| K228N/K247N/N249S/R318Y/R338E/ | K63N/K82N/N84S/R150Y/R170E/ | 10000 | 0 | 0% | 517.0 | 2 |
| R403E/E410N | R233E/E240N | | | | | |
| R318Y/R338E/R403E/E410N/T412V | R150Y/R170E/R233E/E240N/ | 10000 | 0 | 0% | 517.0 | 2 |
| | T242V | | | | | |
| R318Y/R338E/R403E/E410N/T412A | R150Y/R170E/R233E/E240N/ | 10000 | 0 | 0% | 517.0 | 2 |
| | T242A | | | | | |
| R318Y/R338E/R403E/T412A | R150Y/R170E/R233E/T242A | 10000 | 0 | 0% | 517.0 | 2 |
| R318Y/R338E/T412A | R150Y/R170E/T242A | 7661 | 3243 | 42% | 396.1 | 9 |
| R318Y/R338E/E410N/T412V | R150Y/R170E/E240N/T242V | 4871 | 4173 | 86% | 251.8 | 9 |
| N260S/R318Y/R338E/R403E/E410N | N95S/R150Y/R170E/R233E/ | 10000 | 0 | 0% | 517.0 | 2 |
| | E240N | | | | | |
| D104N/K106S/N260S/R318Y/R338E/ | D[104]N/K[106]S/N95S/R150Y/ | 10000 | 0 | 0% | 517.0 | 3 |
| R403E/E410N | R170E/R233E/E240N | | | | | |
| Y155F/N260S/R318Y/R338E/R403E/ | Y[155]F/N95S/R150Y/R170E/ | 9696 | 527 | 5% | 501.3 | 3 |
| E410N | R233E/E240N | | | | | |
| R318Y/R338E/N346D/R403E/E410N | R150Y/R170E/N178D/R233E/ | 10000 | 0 | 0% | 517.0 | 2 |
| | E240N | | | | | |
| Y155F/R318Y/R338E/N346D/R403E/ | Y[155]F/R150Y/R170E/N178D/ | 10000 | 0 | 0% | 517.0 | 2 |
| E410N | R233E/E240N | | | | | |
| K247N/N249S/N260S | K82N/N84S/N95S | 157 | 38 | 24% | 8.1 | 3 |
| Y155F/K247N/N249S/N260S | Y[155]F/K82N/N84S/N95S | 152 | 39 | 26% | 7.9 | 3 |
| D104N/K106S/K247N/N249S/N260S | D[104]N/K[106]S/K82N/N84S/ | 1262 | 40 | 3% | 65.3 | 2 |
| | N95S | | | | | |
| D104N/K106S/Y155F/K247N/N249S/ | D[104]N/K[106]S/Y[155]F/K82N/ | 692 | 84 | 12% | 35.8 | 2 |
| N260S | N84S/N95S | | | | | |
| K247N/N249S/N260S/R318Y/R338E/ | K82N/N84S/N95S/R150Y/R170E/ | 5560 | 3872 | 70% | 287.5 | 3 |
| R403E/E410N | R233E/E240N | | | | | |
| Y155F/N260S/N346D | Y[155]F/N95S/N178D | 1382 | 477 | 35% | 71.4 | 2 |
| R318Y/R338E/T343R/R403E/E410N | R150Y/R170E/T175R/R233E/ | 10000 | 0 | 0% | 517.0 | 4 |
| | E240N | | | | | |
| R338E/T343R | R170E/T175R | 12 | 6 | 46% | 0.6 | 4 |
| T343R/N346Y | T175R/N178Y | 3 | 1 | 32% | 0.1 | 4 |
| R318Y/R338E/N346Y/R403E/E410N | R150Y/R170E/N178Y/R233E/ | 10000 | 0 | 0% | 517.0 | 2 |
| | E240N | | | | | |
| R318Y/R338E/T343R/N346Y/R403E/ | R150Y/R170E/T175R/N178Y/ | 10000 | 0 | 0% | 517.0 | 2 |
| E410N | R233E/E240N | | | | | |
| T343R/N346D | T175R/N178D | 22 | 4 | 18% | 1.1 | 2 |
| R318Y/R338E/T343R/N346D/R403E/ | R150Y/R170E/T175R/N178D/ | 10000 | 0 | 0% | 517.0 | 2 |
| E410N | R233E/E240N | | | | | |
| R318Y/R338E/Y345A/R403E/E410N | R150Y/R170E/Y177A/R233E/ | 10000 | 0 | 0% | 517.0 | 2 |
| | E240N | | | | | |
| R318Y/R338E/Y345A/N346D/R403E/ | R150Y/R170E/Y177A/N178D/ | 10000 | 0 | 0% | 517.0 | 2 |
| E410N | R233E/E240N | | | | | |

* A K_{0.5} value of 999 nM indicates the lower limit value for those variants with less than 10% inhibition under the conditions of the standard assay (35 nM-0 nM AT-III).
* Variants with >50% of WT k_{cat}/K_{M} (see Example 4) and initially given a K_{0.5} value of 999 nM were retested at higher AT-III concentrations, expanding in the sensitivity of the assay.
* A K_{0.5} value of 10000 nM indicates the lower limit value for those variants with less than 10% inhibition under the conditions of the expanded sensitivity assay (1000 nM-0 nM AT-III and 5000-0 nM AT-III).

C. Determination of the Second-Order Rate Constant (kₐₚₚ) for Inhibition of FIXa by the Antithrombin/UFH Complex

Additional experiments were performed to measure the second-order rate constant for inhibition (kₐₚₚ) of FIXa variants by AT-III/UFH using the same assay as described above in Example 5B with minor modifications. This method is more amenable to evaluating the second-order rate constants for multiple variants concurrently than the traditional competitive kinetic or discontinuous methods (see e.g., Olson et al. (2004) J. Thromb. Haemost. 92(5):929-939).

For inhibition reactions in the presence of UFH, a 1000 nM solution of AT-III/UFH were prepared by dilution of a 20 μM stock of plasma purified human AT-III (Molecular Innovations) into a solution of excess UFH (2 μM) in a 1.0 mL volume of 1× Buffer A (50 mM Tris, 100 mM NaCl, 10 mM CaCl₂), 0.01% Tween-20, pH 7.4). AT-III/UFH solutions were incubated for 30 minutes at room temperature prior to being serially diluted 2.0-fold in a 96 deep-well polypropylene plate with a final volume of 500 μL 1× Buffer A containing 2 μM UFH. The final dilutions of AT-III for the modified kₐₚₚ assay ranged from 500 nM-0 nM (i.e., rows A-H). A total of 35 of each AT-III dilution was aliquoted into their respective rows of a 96-well V-bottom storage plate to fill all columns (i.e., 1-12). FIXa variants were initially diluted to 100 nM in 1× Buffer A. Subsequently, 50 μL of each 100 nM FIXa variant was diluted to a concentration of 2.0 nM in 2.5 mL of 1× Buffer A and then 70 μL of this solution was aliquoted into a 96-well V-bottom storage plate according to the same predefined plate map as above (4 FIXa variants per plate).

Assay reactions were initiated using a BioMek FX liquid handling system programmed to dispense 35 μL of the FIXa solutions into the plates containing 35 of each dilution of AT-III/UFH per well for a total of two duplicate assay plates for each FIXa variant. The final inhibition assay conditions were: 1.0 nM FIXa and AT-III dilutions ranging from 500 nM to 0 nM in 1 μM UFH so that the heparin remained in excess. Inhibition reactions were further incubated for various times at room temperature (˜25° C.) depending on the expected inhibition rate constant and adjusted so that >90% inhibition could be reached at the highest concentration of AT-III in the assay (500 nM). Typical incubation times were determined specifically for each variant, or class of variants, but generally followed the incubation times outlined in Table 32, below.

TABLE 32
Assay Incubation Times Based on Expected kₐₚₚ Values

| Expected kₐₚₚ | FIXa/ATIII |
|---|---|
| (M⁻¹s⁻¹) | Incubation (sec) |
| 1.0E−07 | 10 |
| 1.0E−06 | 30 |
| 1.0E−05 | 120 |
| 1.0E−04 | 600 |
| 1.0E−03 | 3600 |
| 1.0E−02 | 7200 |

Following the desired incubation time a 40 μL aliquot of the reaction was transferred by the BioMek FX to a 96-well black half-area plate containing 20 μL of 2.5 mM Mesyl-D-CHG-Gly-Arg-AMC per well in assay Buffer C (50 mM Tris, 100 mM NaCl, 10 mM CaCl₂), 0.01% Tween-20, pH 7.4, 82% ethylene glycol and 5 mg/mL polybrene). Polybrene (hexadimethrine bromide) at a final concentration of 5 mg/mL was added to Buffer C to quench the AT-III/UFH reaction. Residual activity of FIXa was assessed by following the initial rates of substrate cleavage for 60 minutes in a fluorescence reader set to 25° C. The final assay conditions for determination of residual activity were 0.67 nM FIXa variant, 0.83 mM Mesyl-D-CHG-Gly-Arg-AMC, 30% ethylene glycol and 5 mg/mL polybrene in 50 mM Tris, 100 mM NaCl, 10 mM CaCl₂), 0.01% Tween-20, pH 7.4. Data analyses to calculate the K_{0.5} value were performed in a similar manner as that described above for AT-III/UFH inhibition assays in Example 5B using the ActivityBase software package and the XE Runner data analysis module (IDBS Software). Using the assay set-up outlined in Example 5B under psuedo-1st-order conditions and testing various incubation times it is thus possible to calculate the apparent second-order rate constant for inhibition by AT-III (kₐₚₚ) using the following equations:

$$\begin{matrix}{k}_{app} = \frac{{k}_{obs}}{( \frac{[ \text{AT-III]}}{S . I .} )} & Equation ( 1 ) \\ {k}_{obs} = \frac{ln ( 2 )}{{t}_{1 / 2}} & Equation ( 2 )\end{matrix}$$

Given that the fit value for K_{0.5}=[AT-III] at t_{1/2} (defined by the time of the assay) all the necessary values are available to calculate k_{obs} and thus the kₐₚₚ for inhibition of a given FIXa variant by AT-III. The calculated kₐₚₚ value does not take into account any potential effects of changes in the stoichiometry of inhibition (S.I.), which is given a constant value of 1.2 in the present calculations as this value reflects what is typically reported in the literature (see e.g., Olson et al. (2004) J. Thromb. Haemost. 92(5):929-939).

Table 33 provides the results of the second-order rate assays that were performed using AT-III/UFH. The results are presented both as the fitted kₐₚₚ parameter and as a representation of the extent of AT-III resistance for each variant compared to the wild-type FIXa expressed as a ratio of their fitted kₐₚₚ values (kₐₚₚ wild-type/kₐₚₚ variant). Several FIXa variants exhibited greater than 10,000-20,000 fold increased resistance to AT-III compared to wild-type FIXa. For example, FIXa-R318A, FIXa-R318Y, FIXa-R338A/R403A, FIXa-R318Y/R338E/R403E, FIXa-R318Y/R338E/R403E, FIXa-K247N/N249S/R318Y/R338E/R403E, FIXa-R318Y/R338E/R403E, FIXa-K228N/I251S/R318Y/R338E/R403E/E410N, FIXa-R318Y/R338E/E410N and FIXa-R318Y/R338E/R403E/E410N are among this group, which exhibited significant resistance to AT-III.

TABLE 33
Second-Order Rate Constant for Inhibition by AT-III/UFH

| Mutation | Mutation | kₐₚₚ | ±S.D. | | k_{app-wt}/ | |
|---|---|---|---|---|---|---|
| (Mature FIX Numbering) | (Chymotrypsin Numbering) | (M⁻¹s⁻¹) | (M⁻¹s⁻¹) | % CV | kₐₚₚ₋ₘᵤₜ | n |
| BeneFIX ® Coagulation | BeneFIX ® Coagulation | 1.6E+07 | 1.7E+07 | 105% | 1 | 8 |
| FIX (T148A) | FIX (T[148]A) | | | | | |
| Catalyst Biosciences WT | Catalyst Biosciences WT | 2.4E+07 | 8.0E+06 | 33% | 1 | 4 |
| T148A | T[148]A | 1.6E+07 | 1.1E+07 | 69% | 1 | 4 |
| D203N/F205T | D39N/F41T | 8.1E+05 | 5.3E+05 | 66% | 30 | 3 |
| D85N/D203N/F205T | D[85]N/D39N/F41T | 2.7E+06 | 4.5E+05 | 17% | 9 | 2 |
| N260S | N95S | 1.1E+06 | 2.1E+04 | 2% | 21 | 2 |
| D104N/K106S/N260S | D[104]N/K[106]S/N95S | 7.0E+06 | 1.9E+06 | 27% | 3 | 3 |
| R318A | R150A | 6.9E+05 | 5.6E+04 | 8% | 35 | 2 |
| R318E | R150E | 1.6E+04 | 1.2E+03 | 7% | 1,452 | 2 |
| R318Y | R150Y | 6.4E+05 | 3.5E+05 | 55% | 37 | 5 |
| R312Y | R143Y | 2.3E+05 | 4.5E+04 | 19% | 102 | 3 |
| R403A | R233A | 1.4E+06 | 3.1E+05 | 23% | 18 | 2 |
| R403E | R233E | 1.1E+05 | 2.4E+04 | 21% | 209 | 2 |
| K400E | K230E | 4.1E+05 | 3.3E+04 | 8% | 58 | 2 |
| K293E | K126E | 1.2E+06 | 8.4E+04 | 7% | 20 | 2 |
| R338E | R170E | 2.7E+05 | 1.7E+05 | 64% | 88 | 3 |
| R338A/R403A | R170A/R233A | 8.4E+05 | 4.6E+04 | 5% | 28 | 2 |
| R338E/R403E | R170E/R233E | 6.8E+04 | 1.9E+04 | 28% | 353 | 2 |
| K293A/R403A | K126A/R233A | 8.1E+04 | 1.5E+04 | 18% | 294 | 2 |
| K293A/R338A/R403A | K126A/R170A/R233A | 4.7E+04 | 7.9E+03 | 17% | 511 | 2 |
| K293E/R338E/R403E | K126E/R170E/R233E | 3.1E+04 | 6.3E+03 | 20% | 768 | 2 |
| R318A/R403A | R150A/R233A | 1.7E+04 | 4.7E+03 | 27% | 1,390 | 2 |
| R318Y/E410N | R150Y/E240N | 1.1E+06 | 7.9E+03 | 1% | 22 | 2 |
| R338E/E410N | R170E/E240N | 6.3E+06 | 7.4E+06 | 117% | 4 | 10 |
| R338E/R403E/E410N | R170E/R233E/E240N | 1.3E+05 | 1.5E+05 | 115% | 180 | 14 |
| Y155F/R338E/R403E/E410N | Y[155]F/R170E/R233E/E240N | 3.2E+04 | 1.7E+03 | 5% | 755 | 2 |
| R318Y/R338E/R403E | R150Y/R170E/R233E | 1.2E+03 | 9.9E+02 | 80% | 19,396 | 7 |
| Y155F/R318Y/R338E/R403E | Y[155]F/R150Y/R170E/R233E | 1.0E+03 | 5.4E+01 | 5% | 23,242 | 2 |
| D203N/F205T/K228N | D39N/F41T/K63N | 1.1E+06 | 3.7E+05 | 33% | 21 | 2 |
| D203N/F205T/E410N | D39N/F41T/E240N | 2.0E+06 | 2.1E+05 | 10% | 12 | 2 |
| D203N/F205T/R338E | D39N/F41T/R170E | 3.6E+05 | 2.8E+04 | 8% | 66 | 2 |
| D203N/F205T/R338A | D39N/F41T/R170A | 8.6E+05 | 1.6E+05 | 18% | 28 | 2 |
| D203N/F205T/R318Y | D39N/F41T/R150Y | 6.1E+04 | 2.0E+04 | 33% | 391 | 2 |
| D203N/F205T/R338E/R403E | D39N/F41T/R170E/R233E | 2.0E+03 | n.d. | n.d. | 12,250 | 1 |
| K228N/R318Y | K63N/R150Y | 1.2E+06 | 2.1E+05 | 17% | 19 | 2 |
| K228N/R338E/R403E | K63N/R170E/R233E | 4.2E+04 | 1.3E+04 | 31% | 567 | 2 |
| R403E/E410N | R233E/E240N | 4.8E+06 | 2.5E+06 | 53% | 5 | 5 |
| R318Y/R338E/E410N | R150Y/R170E/E240N | 2.8E+05 | 2.4E+05 | 85% | 84 | 8 |
| D104N/K106S/R318Y/R338E/ | D[104]N/K[106]S/R150Y/R170E/ | 2.1E+05 | 4.2E+04 | 20% | 113 | 2 |
| E410N | E240N | | | | | |
| Y155F/R318Y/R338E/E410N | Y[155]F/R150Y/R170E/E240N | 4.5E+05 | 6.9E+04 | 15% | 53 | 2 |
| K228N/R318Y/E410N | K63N/R150Y/E240N | 1.9E+06 | n.d. | n.d. | 12 | 1 |
| R318Y/R403E/E410N | R150Y/R233E/E240N | 2.8E+04 | 1.8E+04 | 63% | 856 | 6 |
| Y155F/R318Y/R403E/E410N | Y[155]F/R150Y/R233E/E240N | 8.1E+03 | 1.4E+02 | 2% | 2,963 | 2 |
| R318Y/R338E/R403E/E410N | R150Y/R170E/R233E/E240N | 3.2E+03 | 2.0E+03 | 63% | 7,385 | 6 |
| A103N/N105S/R318Y/R338E/ | A[103]N/N[105]S/R150Y/R170E/ | 2.6E+03 | 1.7E+02 | 7% | 9,060 | 2 |
| R403E/E410N | R233E/E240N | | | | | |
| D104N/K106S/R318Y/R338E/ | D[104]N/K[106]S/R150Y/R170E/ | 3.9E+03 | 1.6E+01 | 0% | 6,154 | 2 |
| R403E/E410N | R233E/E240N | | | | | |
| Y155F/R318Y/R338E/R403E/ | Y[155]F/R150Y/R170E/R233E/ | 3.2E+03 | 8.1E+02 | 25% | 7,464 | 3 |
| E410N | E240N | | | | | |
| A103N/N105S/Y155F/R318Y/ | A[103]N/N[105]S/Y[155]F/ | 3.2E+03 | 6.7E+00 | 0% | 7,531 | 2 |
| R338E/R403E/E410N | R150Y/R170E/R233E/E240N | | | | | |
| D104N/K106S/Y155F/R318Y/ | D[104]N/K[106]S/Y[155]F/ | 2.9E+03 | 1.8E+02 | 6% | 8,147 | 2 |
| R338E/R403E/E410N | R150Y/R170E/R233E/E240N | | | | | |
| D203N/F205T/R318Y/E410N | D39N/F41T/R150Y/E240N | 5.3E+04 | 5.8E+03 | 11% | 454 | 3 |
| N346D | N178D | 3.4E+06 | 1.6E+06 | 48% | 7 | 4 |
| Y155F/N346D | Y[155]F/N178D | 4.0E+06 | 5.4E+05 | 13% | 6 | 2 |
| N346Y | N178Y | 8.4E+05 | n.d. | n.d. | 28 | 1 |
| Y345T | Y177T | 1.8E+06 | 7.8E+03 | 0% | 13 | 2 |
| T343R | T175R | 4.2E+06 | 1.0E+04 | 0% | 6 | 2 |
| T343Q | T175Q | 2.1E+06 | 5.4E+05 | 25% | 11 | 2 |
| T343R/Y345T | T175R/Y177T | 5.0E+06 | 1.8E+05 | 4% | 5 | 2 |
| R318Y/R338E | R150Y/R170E | 6.2E+05 | 5.4E+04 | 9% | 39 | 2 |
| K228N/R318Y/R338E/R403E/ | K63N/R150Y/R170E/R233E/ | 2.9E+03 | 2.2E+02 | 7% | 8,212 | 2 |
| E410N | E240N | | | | | |
| Y155F/K228N/R318Y/R338E/ | Y[155]F/K63N/R150Y/R170E/ | 4.6E+03 | 6.1E+02 | 13% | 5,161 | 2 |
| R403E/E410N | R233E/E240N | | | | | |
| D85N/K228N/R318Y/R338E/ | D[85]N/K63N/R150Y/R170E/ | 3.0E+03 | 3.2E+02 | 11% | 7,932 | 2 |
| R403E/E410N | R233E/E240N | | | | | |
| I251S/R318Y/R338E/R403E/ | I86S/R150Y/R170E/R233E/ | 3.0E+03 | 3.5E+02 | 12% | 7,940 | 2 |
| E410N | E240N | | | | | |
| D104N/K106S/I251S/R318Y/ | D[104]N/K[106]S/I86S/R150Y/ | 5.7E+03 | 8.4E+02 | 15% | 4,225 | 2 |
| R338E/R403E/E410N | R170E/R233E/E240N | | | | | |
| Y155F/I251S/R318Y/R338E/ | D[104]N/K[106]S/I86S/R150Y/ | 3.3E+03 | 1.4E+02 | 4% | 7,306 | 2 |
| R403E/E410N | R170E/R233E/E240N | | | | | |
| I251S/R318Y/R338E/E410N | I86S/R150Y/R170E/E240N | 2.4E+05 | 2.1E+05 | 89% | 100 | 6 |
| D104N/K106S/I251S/R318Y/ | D[104]N/K[106]S/I86S/R150Y/ | 3.2E+03 | 4.5E+02 | 14% | 7,567 | 2 |
| R338E/E410N | R170E/E240N | | | | | |
| K247N/N249S/R318Y/R338E/ | K82N/N84S/R150Y/R170E/ | 2.0E+03 | 1.0E+03 | 53% | 12,122 | 2 |
| R403E/E410N | R233E/E240N | | | | | |
| Y155F/K247N/N249S/R318Y/ | Y[155]F/K82N/N84S/R150Y/ | 1.6E+03 | 5.9E+02 | 37% | 15,058 | 4 |
| R338E/R403E/E410N | R170E/R233E/E240N | | | | | |
| A103N/N105S/K247N/N249S/ | A[103]N/N[105]S/K82N/N84S/ | 1.7E+03 | 2.4E+02 | 14% | 14,063 | 3 |
| R318Y/R338E/R403E/E410N | R150Y/R170E/R233E/E240N | | | | | |
| D104N/K106S/K247N/N249S/ | D[104]N/K[106]S/K82N/N84S/ | 3.1E+03 | 7.6E+02 | 24% | 7,646 | 3 |
| R318Y/R338E/R403E/E410N | R150Y/R170E/R233E/E240N | | | | | |
| D104N/K106S/Y155F/K247N/ | D[104]N/K[106]S/Y[155]F/ | 1.0E+03 | 2.8E+02 | 28% | 23,776 | 6 |
| N249S/R318Y/R338E/R403E/ | K82N/N84S/R150Y/R170E/ | | | | | |
| E410N | R233E/E240N | | | | | |
| K247N/N249S/R318Y/R338E/ | K82N/N84S/R150Y/R170E/ | 8.6E+05 | 1.2E+05 | 14% | 28 | 2 |
| E410N | E240N | | | | | |
| Y155F/K247N/N249S/R318Y/ | Y[155]F/K82N/N84S/R150Y/ | 1.8E+05 | 2.2E+04 | 13% | 136 | 2 |
| R338E/E410N | R170E/E240N | | | | | |
| R318Y/R338E/R403E/E410S | R150Y/R170E/R233E/E240S | 1.6E+03 | 1.1E+03 | 64% | 14,483 | 7 |
| R318Y/R338E/E410S | R150Y/R170E/E240S | 7.2E+05 | 4.8E+05 | 66% | 33 | 2 |
| K228N/K247N/N249S/R318Y/ | K63N/K82N/N84S/R150Y/ | 1.1E+03 | 4.5E+02 | 41% | 21,766 | 12 |
| R338E/R403E/E410N | R170E/R233E/E240N | | | | | |
| D104N/K106S/K228N/K247N/ | D[104]N/K[106]S/K63N/K82N/ | 6.8E+02 | 3.3E+02 | 48% | 35,018 | 4 |
| N249S/R318Y/R338E/R403E/ | N84S/R150Y/R170E/R233E/ | | | | | |
| E410N | E240N | | | | | |
| Y155F/K228N/K247N/N249S/ | Y[155]F/K63N/K82N/N84S/ | 1.1E+03 | 3.9E+01 | 4% | 21,856 | 4 |
| R318Y/R338E/R403E/E410N | R150Y/R170E/R233E/E240N | | | | | |
| R318Y/R338E/R403E/E410N/ | R150Y/R170E/R233E/E240N/ | 2.9E+03 | 5.4E+02 | 19% | 8,296 | 5 |
| T412V | T242V | | | | | |
| R318Y/R338E/R403E/E410N/ | R150Y/R170E/R233E/E240N/ | 3.8E+03 | 1.2E+03 | 31% | 6,322 | 5 |
| T412A | T242A | | | | | |
| R318Y/R338E/R403E/T412A | R150Y/R170E/R233E/T242A | 1.6E+03 | 3.8E+02 | 23% | 14,529 | 2 |
| R318Y/R338E/T412A | R150Y/R170E/T242A | 3.5E+05 | 7.2E+04 | 21% | 69 | 3 |
| R318Y/R338E/E410N/T412V | R150Y/R170E/E240N/T242V | 3.9E+05 | 2.6E+04 | 7% | 61 | 2 |
| N260S/R318Y/R338E/R403E/ | N95S/R150Y/R170E/R233E/ | 4.4E+03 | 8.5E+02 | 19% | 5,407 | 2 |
| E410N | E240N | | | | | |
| D104N/K106S/N260S/R318Y/ | D[104]N/K[106]S/N95S/R150Y/ | 2.1E+03 | 3.9E+02 | 18% | 11,173 | 2 |
| R338E/R403E/E410N | R170E/R233E/E240N | | | | | |
| Y155F/N260S/R318Y/R338E/ | Y[155]F/N95S/R150Y/R170E/ | 2.1E+03 | 2.4E+02 | 11% | 11,456 | 2 |
| R403E/E410N | R233E/E240N | | | | | |
| R318Y/R338E/N346D/R403E/ | R150Y/R170E/N178D/R233E/ | 1.1E+03 | 5.5E+02 | 49% | 21,504 | 6 |
| E410N | E240N | | | | | |
| Y155F/R318Y/R338E/N346D/ | Y[155]F/R150Y/R170E/N178D/ | 1.6E+03 | 6.6E+02 | 41% | 14,831 | 3 |
| R403E/E410N | R233E/E240N | | | | | |
| D104N/K106S/K247N/N249S/ | D[104]N/K[106]S/K82N/N84S/ | 1.7E+06 | 8.7E+04 | 5% | 14 | 2 |
| N260S | N95S | | | | | |
| D104N/K106S/Y155F/K247N/ | D[104]N/K[106]S/Y[155]F/ | 3.2E+06 | 2.1E+05 | 6% | 7 | 2 |
| N249S/N260S | K82N/N84S/N95S | | | | | |
| K247N/N249S/N260S/R318Y/ | K82N/N84S/N95S/R150Y/ | 1.3E+03 | 3.8E+02 | 30% | 18,567 | 2 |
| R338E/R403E/E410N | R170E/R233E/E240N | | | | | |
| Y155F/K247N/N249S/N260S/ | Y[155]F/K82N/N84S/N95S/ | 4.3E+02 | 3.8E+00 | 1% | 55,342 | 4 |
| R318Y/R338E/R403E/E410N | R150Y/R170E/R233E/E240N | | | | | |
| R318Y/R338E/T343R/R403E/ | R150Y/R170E/T175R/R233E/ | 3.2E+04 | 2.2E+04 | 69% | 749 | 6 |
| E410N | E240N | | | | | |
| Y155F/R318Y/R338E/T343R/ | Y[155]F/R150Y/R170E/T175R/ | 8.6E+03 | 5.4E+03 | 63% | 2,774 | 6 |
| R403E/E410N | R233E/E240N | | | | | |
| D104N/K106S/R318Y/R338E/ | D[104]N/K[106]S/R150Y/R170E/ | 9.1E+03 | 2.4E+03 | 27% | 2,636 | 4 |
| T343R/R403E/E410N | T175R/R233E/E240N | | | | | |
| R338E/T343R | R170E/T175R | 3.4E+06 | 4.8E+05 | 14% | 7 | 2 |
| T343R/N346Y | T175R/N178Y | 4.2E+06 | 4.0E+06 | 95% | 6 | 4 |
| R318Y/R338E/N346Y/R403E/ | R150Y/R170E/N178Y/R233E/ | 2.8E+03 | 4.4E+02 | 16% | 8,498 | 2 |
| E410N | E240N | | | | | |
| R318Y/R338E/T343R/N346Y/ | R150Y/R170E/T175R/N178Y/ | 1.1E+04 | 4.3E+03 | 37% | 2,086 | 4 |
| R403E/E410N | R233E/E240N | | | | | |
| T343R/N346D | T175R/N178D | 1.3E+06 | 2.3E+05 | 18% | 18 | 2 |
| R318Y/R338E/T343R/N346D/ | R150Y/R170E/T175R/N178D/ | 5.1E+03 | 3.7E+01 | 1% | 4,726 | 2 |
| R403E/E410N | R233E/E240N | | | | | |
| R318Y/R338E/Y345A/R403E/ | R150Y/R170E/Y177A/R233E/ | 7.9E+03 | 1.2E+03 | 16% | 3,015 | 2 |
| E410N | E240N | | | | | |
| Y155F/K247N/N249S/R318Y/ | Y[155]F/K82N/N84S/R150Y/ | 8.1E+02 | 1.6E+02 | 20% | 29,512 | 4 |
| R338E/R403E | R170E/R233E | | | | | |
| K247N/N249S/R318Y/R338E/ | K82N/N84S/R150Y/R170E/ | 3.1E+02 | 2.1E+02 | 67% | 76,373 | 4 |
| R403E | R233E | | | | | |
| Y155F/K247N/N249S/R318Y/ | Y[155]F/K82N/N84S/R150Y/ | 7.3E+03 | 2.0E+01 | 0% | 3,291 | 2 |
| R403E/E410N | R233E/E240N | | | | | |
| K247N/N249S/R318Y/R403E/ | K82N/N84S/R150Y/R233E/ | 2.7E+03 | 9.3E+02 | 35% | 8,942 | 6 |
| E410N | E240N | | | | | |
| Y155F/K247N/N249S/R338E/ | Y[155]F/K82N/N84S/R170E/ | 4.2E+04 | 4.3E+02 | 1% | 572 | 2 |
| R403E/E410N | R233E/E240N | | | | | |
| K247N/N249S/R338E/R403E/ | K82N/N84S/R170E/R233E/ | 2.1E+04 | 1.5E+03 | 7% | 1,148 | 2 |
| E410N | E240N | | | | | |
| R318Y/R338E/T343R/R403E | R150Y/R170E/T175R/R233E | 5.8E+03 | 8.6E+02 | 15% | 4,118 | 2 |
| Y155F/R318Y/R338E/T343R/ | Y[155]F/R150Y/R170E/T175R/ | 2.8E+03 | 3.8E+02 | 14% | 8,515 | 6 |
| R403E | R233E | | | | | |
| R318Y/R338E/T343R/E410N | R150Y/R170E/T175R/E240N | 5.4E+05 | 3.2E+05 | 58% | 44 | 8 |
| Y155F/R318Y/R338E/T343R/ | Y[155]F/R150Y/R170E/T175R/ | 7.8E+05 | 6.1E+05 | 79% | 31 | 4 |
| E410N | E240N | | | | | |
| R318Y/T343R/R403E/E410N | R150Y/T175R/R233E/E240N | 9.3E+04 | 1.2E+04 | 13% | 257 | 2 |
| Y155F/R318Y/T343R/R403E/ | Y[155]F/R150Y/T175R/R233E/ | 5.5E+04 | 7.8E+03 | 14% | 436 | 4 |
| E410N | E240N | | | | | |
| R338E/T343R/R403E/E410N | R170E/T175R/R233E/E240N | 3.4E+05 | 2.7E+03 | 1% | 70 | 2 |
| Y155F/R338E/T343R/R403E/ | Y[155]F/R170E/T175R/R233E/ | 2.8E+05 | 1.7E+04 | 6% | 85 | 4 |
| E410N | E240N | | | | | |
| Y155F/K247N/N249S/R318Y/ | Y[155]F/K82N/N84S/R150Y/ | 8.7E+03 | 1.9E+03 | 22% | 2,733 | 8 |
| R338E/T343R/R403E/E410N | R170E/T175R/R233E/E240N | | | | | |
| K247N/N249S/R318Y/R338E/ | K82N/N84S/R150Y/R170E/ | 9.6E+03 | 2.4E+03 | 25% | 2,499 | 4 |
| T343R/R403E/E410N | T175R/R233E/E240N | | | | | |
| K228N/I251S/R318Y/R338E/ | K63N/I86S/R150Y/R170E/ | 9.0E+02 | 2.2E+02 | 25% | 26,598 | 4 |
| R403E/E410N | R233E/E240N | | | | | |
| Y155F/K228N/I251S/R318Y/ | Y[155]F/K63N/I86S/R150Y/ | 1.3E+03 | 2.8E+02 | 21% | 17,778 | 6 |
| R338E/R403E/E410N | R170E/R233E/E240N | | | | | |
| N260S/R318Y/R338E/T343R/ | N95S/R150Y/R170E/T175R/ | 2.6E+03 | 5.6E+02 | 22% | 9,317 | 4 |
| R403E/E410N | R233E/E240N | | | | | |
| Y155F/N260S/R318Y/R338E/ | Y[155]F/N95S/R150Y/R170E/ | 2.6E+03 | 6.6E+02 | 25% | 9,148 | 4 |
| T343R/R403E/E410N | T175R/R233E/E240N | | | | | |
| K228N/K247N/N249S/R318Y/ | K63N/K82N/N84S/R150Y/ | 5.3E+03 | 1.8E+03 | 34% | 4,468 | 10 |
| R338E/T343R/R403E/E410N | R170E/T175R/R233E/E240N | | | | | |
| Y155F/K228N/K247N/N249S/ | Y[155]F/K63N/K82N/N84S/ | 2.2E+03 | 1.4E+03 | 62% | 10,758 | 4 |
| R318Y/R338E/T343R/R403E/ | R150Y/R170E/T175R/R233E/ | | | | | |
| E410N | E240N | | | | | |
| Y155F/R338E/T343R/R403E | Y[155]F/R170E/T175R/R233E | 9.3E+04 | 1.2E+04 | 13% | 257 | 4 |
| R338E/T343R/R403E | R170E/T175R/R233E | 1.9E+05 | 7.1E+02 | 0% | 125 | 2 |
| Y155F/R338E/T343R/R403E/ | Y[155]F/R170E/T175R/R233E/ | 2.2E+05 | 2.6E+04 | 12% | 110 | 6 |
| E410S | E240S | | | | | |
| Y155F/N260S/R338E/T343R/ | Y[155]F/N95S/R170E/T175R/ | 4.0E+04 | 7.6E+03 | 19% | 601 | 4 |
| R403E | R233E | | | | | |
| Y155F/I251S/R338E/T343R/ | Y[155]F/I86S/R170E/T175R/ | 1.6E+05 | 1.5E+04 | 9% | 146 | 2 |
| R403E | R233E | | | | | |
| R318Y/R338E/T343R/R403E/ | R150Y/R170E/T175R/R233E/ | 9.9E+03 | 2.9E+03 | 30% | 2,417 | 22 |
| E410S | E240S | | | | | |
| Y155F/K247N/N249S/T343R/ | Y[155]F/K82N/N84S/T175R/ | 1.4E+05 | 2.3E+04 | 16% | 168 | 4 |
| R403E | R233E | | | | | |
| Y155F/K247N/N249S/R318Y/ | Y[155]F/K82N/N84S/R150Y/ | 2.3E+03 | 1.7E+02 | 8% | 10,415 | 2 |
| R338E/T343R/R403E | R170E/T175R/R233E | | | | | |
| K247N/N249S/R318Y/R338E/ | K82N/N84S/R150Y/R170E/ | 1.7E+03 | 2.0E+02 | 12% | 14,156 | 4 |
| T343R/R403E | T175R/R233E | | | | | |
| Y155F/K247N/N249S/R338E/ | Y[155]F/K82N/N84S/R170E/ | 8.9E+04 | 1.1E+04 | 13% | 268 | 4 |
| T343R/R403E/E410N | T175R/R233E/E240N | | | | | |
| K247N/N249S/R338E/T343R/ | K82N/N84S/R170E/T175R/ | 8.6E+04 | 1.1E+04 | 13% | 276 | 4 |
| R403E/E410N | R233E/E240N | | | | | |
| Y155F/K247N/N249S/R318Y/ | Y[155]F/K82N/N84S/R150Y/ | 2.7E+04 | 1.4E+04 | 50% | 889 | 4 |
| R338E | R170E | | | | | |
| Y155F/K247N/N249S/R318Y/ | Y[155]F/K82N/N84S/R150Y/ | 4.0E+05 | 2.9E+05 | 72% | 60 | 8 |
| T343R | T175R | | | | | |
| Y155F/K247N/N249S/R318Y/ | Y[155]F/K82N/N84S/R150Y/ | 2.1E+03 | 5.3E+01 | 2% | 11,125 | 2 |
| R403E | R233E | | | | | |
| Y155F/K247N/N249S/R318Y/ | Y[155]F/K82N/N84S/R150Y/ | 1.3E+05 | 9.5E+04 | 75% | 188 | 6 |
| E410N | E240N | | | | | |
| Y155F/K247N/N249S/R338E/ | Y[155]F/K82N/N84S/R170E/ | 1.3E+04 | 1.0E+03 | 8% | 1,819 | 2 |
| R403E | R233E | | | | | |
| Y155F/K247N/N249S/R338E/ | Y[155]F/K82N/N84S/R170E/ | 1.2E+07 | 6.2E+06 | 51% | 2 | 4 |
| T343R | T175R | | | | | |
| Y155F/K247N/N249S/R318Y/ | Y[155]F/K82N/N84S/R150Y/ | 2.2E+05 | 1.0E+05 | 45% | 107 | 4 |
| R338E/T343R/E410N | R170E/T175R/E240N | | | | | |
| K247N/N249S/R318Y/R338E/ | K82N/N84S/R150Y/R170E/ | 2.1E+05 | 8.2E+04 | 39% | 114 | 4 |
| T343R/E410N | T175R/E240N | | | | | |
| Y155F/K247N/N249S/R318Y/ | Y[155]F/K82N/N84S/R150Y/ | 2.8E+04 | 5.6E+03 | 20% | 842 | 4 |
| T343R/R403E/E410N | T175R/R233E/E240N | | | | | |
| K247N/N249S/R318Y/T343R/ | K82N/N84S/R150Y/T175R/ | 2.5E+04 | 8.0E+03 | 32% | 962 | 6 |
| R403E/E410N | R233E/E240N | | | | | |
| Y155F/K247N/N249S/R338E/ | Y[155]F/K82N/N84S/R170E/ | 2.9E+06 | 2.2E+06 | 77% | 8 | 6 |
| E410N | E240N | | | | | |
| Y155F/K247N/N249S/R318Y/ | Y[155]F/K82N/N84S/R150Y/ | 1.2E+04 | 1.0E+03 | 9% | 2,011 | 4 |
| T343R/R403E | T175R/R233E | | | | | |
| K247N/N249S/R318Y/T343R/ | K82N/N84S/R150Y/T175R/ | 9.8E+03 | 2.5E+03 | 26% | 2,430 | 12 |
| R403E | R233E | | | | | |
| Y155F/K247N/N249S/R318Y/ | Y[155]F/K82N/N84S/R150Y/ | 3.6E+05 | 1.2E+05 | 32% | 66 | 4 |
| T343R/E410N | T175R/E240N | | | | | |
| K247N/N249S/R318Y/T343R/ | K82N/N84S/R150Y/T175R/ | 4.9E+04 | 6.5E+03 | 13% | 487 | 4 |
| E410N | E240N | | | | | |
| Y155F/K247N/N249S/R338E/ | Y[155]F/K82N/N84S/R170E/ | 4.4E+04 | 1.1E+04 | 26% | 549 | 4 |
| T343R/R403E | T175R/R233E | | | | | |
| K247N/N249S/R338E/T343R/ | K82N/N84S/R170E/T175R/ | 5.0E+04 | 1.7E+04 | 35% | 482 | 4 |
| R403E | R233E | | | | | |
| K247N/N249S/R338E/T343R/ | K82N/N84S/R170E/T175R/ | 1.4E+07 | 7.2E+06 | 53% | 2 | 5 |
| E410N | E240N | | | | | |
| Y155F/K247N/N249S/T343R/ | Y[155]F/K82N/N84S/T175R/ | 6.2E+05 | 5.6E+04 | 9% | 39 | 4 |
| R403E/E410N | R233E/E240N | | | | | |
| K247N/N249S/T343R/R403E/ | K82N/N84S/T175R/R233E/ | 4.2E+05 | 8.1E+04 | 19% | 58 | 4 |
| E410N | E240N | | | | | |
| Y155F/R318Y/R338E/T343R | Y[155]F/R150Y/R170E/T175R | 4.4E+05 | 1.9E+05 | 43% | 55 | 6 |
| R318Y/R338E/T343R | R150Y/R170E/T175R | 1.8E+06 | 8.6E+05 | 48% | 13 | 4 |
| Y155F/R318Y/T343R/R403E | Y[155]F/R150Y/T175R/R233E | 1.1E+04 | 9.1E+02 | 8% | 2,114 | 2 |
| Y155F/T343R/R403E/E410N | Y[155]F/T175R/R233E/E240N | 8.8E+05 | 3.3E+03 | 0% | 27 | 2 |
| Y155F/K247N/N249S/R318Y/ | Y[155]F/K82N/N84S/R150Y/ | 3.7E+05 | 1.1E+05 | 28% | 64 | 6 |
| R338E/T343R | R170E/T175R | | | | | |
| K247N/N249S/R318Y/R338E/ | K82N/N84S/R150Y/R170E/ | 3.2E+05 | 1.4E+05 | 44% | 74 | 6 |
| T343R | T175R | | | | | |
| Y155F/K247N/N249S/T343R/ | Y[155]F/K82N/N84S/T175R/ | 3.5E+06 | 4.8E+05 | 14% | 7 | 2 |
| E410N | E240N | | | | | |
| Y155F/K247N/N249S/R403E/ | Y[155]F/K82N/N84S/R233E/ | 1.3E+05 | 3.3E+04 | 26% | 191 | 14 |
| E410N | E240N | | | | | |
| Y155F/R338E/T343R/E410N | Y[155]F/R170E/T175R/E240N | 1.3E+07 | 1.0E+07 | 78% | 2 | 6 |
| R338E/T343R/E410N | R170E/T175R/E240N | 2.0E+07 | 6.3E+06 | 31% | 1 | 4 |
| Y155F/R318Y/T343R/E410N | Y[155]F/R150Y/T175R/E240N | 2.0E+05 | 5.9E+04 | 29% | 118 | 4 |
| R318Y/T343R/E410N | R150Y/T175R/E240N | 1.2E+06 | 1.1E+05 | 9% | 20 | 2 |
| K228N/R150Y/R338E/T343R/ | K63N/R150Y/R170E/T175R/ | 7.1E+03 | 3.3E+02 | 5% | 3,343 | 2 |
| R403E/E410N | R233E/E240N | | | | | |
| K228N/K247N/N249S/R318Y/ | K63N/K82N/N84S/R150Y/ | 1.0E+03 | 2.3E+02 | 22% | 23,389 | 2 |
| R338E/T343R/R403E | R170E/T175R/R233E | | | | | |
| K228N/247N/N249S/R318Y/ | K63N/K82N/N84S/R150Y/ | 6.3E+05 | 1.0E+05 | 17% | 38 | 2 |
| R338E/T343R/E410N | R170E/T175R/E240N | | | | | |
| K228N/K247N/N249S/R318Y/ | K63N/K82N/N84S/R150Y/ | 1.7E+04 | 2.4E+03 | 14% | 1,422 | 2 |
| T343R/R403E/E410N | T175R/R233E/E240N | | | | | |

### Example 6

### Pharmacokinetic and Pharmacodynamic Analysis of FIXa Polypeptides

The pharmacokinetic (PK) and pharmacodynamic (PD) properties of the FIXa variant polypeptides were assessed by measuring the amount of variant FIX in mouse plasma at various time points following intravenous administration. Two assays were used to quantify FIXa in plasma. An ELISA was used to quantify total FIX protein in mouse plasma to assess the pharmacokinetic properties, and a FIX-dependent clotting assay (activated partial thromboplastin time (aPTT) assay using FIX-depleted plasma) was used to quantify the coagulant activity of the FIX polypeptides in plasma, thus assessing the pharmacodynamic properties.

Animals

Male CD-1 mice (30-40 gm), supplied by Charles River Laboratories (Hollister, Calif.) were quarantined for at least 3 days before treatment. For serial PK studies, male CD-1 mice (30-37 gm) were fitted with an indwelling jugular vein cannula. Filtered tap water and food was available ad libitum prior to use in PD or PK experiments.

### A. Dosing and Blood Collection

Mice (N=3 per time point) were administered the FIX polypeptides intravenously (˜1.4 mg/kg for PK studies and ˜400 IU/kg for PD studies, dose volume 2 ml/kg) via the tail vein. At the appropriate time after dosing, animals were anesthetized and blood was drawn (0.5-1 mL) using terminal cardiac puncture into syringes containing citrate. In some experiments where insufficient amount of protein was available, a total of only 4-6 animals were used for serial bleeding at staggered time points; two mice were used for each full time course in order to collect all time points without removing excess blood volume. Blood was sampled in restrained conscious animals by first removing a small amount of blood into a 0.1 mL syringe containing 0.9% saline. A syringe containing 4.5 μl of 0.1M sodium citrate was then attached and 0.05 mL blood was withdrawn into the syringe and the blood was transferred to a 1.5 mL tube. The initial syringe was reattached and 0.07 mL of saline pushed back through the cannula. The cannula was capped until the next time point, when the process was repeated. For all studies, blood samples were centrifuged within 15 minutes of collection (9000 rpm, 8 minutes, 4° C.) and the plasma removed and immediately flash frozen in liquid nitrogen and then stored frozen (−70° C.) pending analysis.

### A. PK Assessment

Citrated blood samples were collected at various times up to 1440 min post dose (i.e., Predose, 2, 4, 10, 30, 60, 120, 240, 360, 480, 960 and 1440 min) by cardiac puncture for terminal experiments or indwelling catheter for serial experiments. Plasma concentrations of rFIX were determined using a factor IX specific ELISA utilizing a matched pair of detection and capture antibodies (#FIX-EIA, Affinity Biologicals, Ancaster, ON). Briefly, an affinity purified polyclonal antibody to FIX is coated onto the wells of a plate. The plates are washed and plasma samples containing FIX are applied. Plasma samples are diluted 1:750 and 1:1500 on the plate. After washing the plate to remove unbound material, a peroxidase conjugated detection antibody to FIX is added to the plate to bind to the captured FIX. After washing the plate to remove unbound conjugated antibody, the peroxidase activity is expressed by incubation with chemiluminescent substrate and read at 425 nM on an EnVision plate reader. The standard curve is linear over the entire concentration range and spans the concentrations of 0.82 pg/ml to 30 ng/ml. The FIX variant itself is used for the standard curve to eliminate differences in the antibody affinity. Each sample is measured on two separate assay plates and those measurements within the range of the standard curve are used to calculate the concentration of FIX variants in the plasma sample.

PD Assessment

The plasma pharmacodynamic activity of rFIX was quantified using an activated partial thromboplastin time (aPTT) assay and FIX deficient human plasma (STACLOT C.K. PREST kit, Diagnostica Stago, Asnieres, France) per the manufacturer's instructions. Briefly, the aPTT assay involves the recalcification of plasma in the presence of cephalin (platelet substitute) and activator (koalin). Using FIX deficient human plasma, the aPTT assay is specific for FIX. The aPTT assay was performed as described in the manufacturers' product insert. Briefly, citrated blood samples were collected at the same time points described for PK assessment. Plasma samples were diluted 1:100 in Tris buffered saline containing 0.1% bovine serum albumin (Probumin, Millipore, Billerica, Mass.). Diluted plasma or standard was combined with FIX deficient human plasma and cephalin/kaolin reagent and incubated for 180 seconds. Coagulation was initiated by the addition of calcium (CaCl₂)). Coagulation time in seconds was measured using a STArt4 instrument (Diagnostica Stago, Asnieres, France). Using a standard curve made from known concentrations of rFIX, plasma FIX concentrations were interpolated from the log concentration VS. log time standard curve plot and then background FIX activity (from pre dose animals) was subtracted. The lower limit of quantification for factor IX activity was ˜10 ng/mL.

PD and PK Data Analysis

PD (aPTT) and PK (ELISA) parameters from mouse studies with rFIX variants were calculated using non-compartmental analysis in WinNonLin (v5.1, Pharsight Corp., Mountain View, Calif.). Both the PD and PK of rFIX variants followed apparent biexponential plasma decay. Select parameters for each variant tested are provided in Table 34 for PD (using the aPTT assay) and Tables 35-36 for PK (using the ELISA assay). Table 35 reflects data for additional FIXa variants and provide new overall averages calculated to include additional experimental replicates (n) for FIXa variants in Table 35. The PD parameters included half-life (terminal, min), MRT (MRT_{0-inf}, min), Area under the curve (AUC) 0-last (min·μg/mL)/Dose (mg/kg); Maximal concentration (Cₘₐₓ; (μg/mL)/Dose (μg/kg), Vd (mL/kg) and Clearance (Cl, mL/min/kg).

### Definitions and Formulae Used to Calculate Pharmacokinetic Parameters

Plasma half-life (the half-life of the FIX polypeptide during the terminal phase of plasma FIX concentration-versus-time profile); T_{1/2β} (calculated as −ln 2 divided by the negative slope during the terminal phase of the log-linear plot of the plasma FIX concentration-versus-time curve); MRT₀₋ₗₐₛₜ is the mean time the FIX polypeptide resides in body; calculated as AUMC₀₋ₗₐₛₜ/AUC₀₋ₗₐₛₜ, (where AUMC₀₋ₗₐₛₜ is the total area under the first moment-versus-time curve and AUC as described subsequently); AUC₀₋ₗₐₛₜ/Dose is calculated as [AUC₍₀₋ₜ₎], where t is the last time point with measurable plasma concentration of the FIX polypeptide divided by the IV dose (mg/kg); AUC_{0-inf}/Dose is calculated as [AUC₍₀₋ₜ₎+Ct/(ln 2/T_{1/2β}], where t is the last time point with measurable plasma concentration of the FIX polypeptide divided by the IV dose (mg/kg); Cₘₐₓ/Dose (μg/mL per mg/kg), where Cₘₐₓ is the time post dose corresponding to the maximal measured plasma FIX concentration; Cl is systemic clearance calculated as (Dose/AUC_{0-inf}); Vₛₛ is the steady state volume of distribution; calculated as MRT*Cl; and V_{z} is the volume of distribution based on the terminal elimination constant (β); calculated as Cl/(ln 2/T_{1/2β}).

TABLE 34
PD properties of FIX variants assessed by aPTT assay

| Mutation | | | | | | | | |
|---|---|---|---|---|---|---|---|---|
| (Mature FIX | | | | Cₘₐₓ/ | | | | |
| numbering) | N | T_{1/2β} | MRT_{0-inf} | dose | AUC_{0-inf} | Cl | Vz | Vss |
| BeneFIX ® | 2 | 296 | 354 | 19.3 | 2641 | 0.41 | 169 | 142 |
| Coagulation FIX | | | | | | | | |
| (T148A) | | | | | | | | |

TABLE 35
PK properties of FIX variants assessed by ELISA

| | | | MRT | | AUC/Dose | AUC/Dose | | |
|---|---|---|---|---|---|---|---|---|
| Mutation | N | T½_{β} | 0-inf | Cmax/Dose | 0-last | 0-inf | Vz | Cl |
| BeneFIX ® Coagulation FIX | 3 | 314 ± 128 | 366 ± 105 | 9.1 ± 1.5 | 1298 ± 298 | 1522 ± 158 | 308 ± 160 | 0.74 ± 0.06 |
| (T148A) | | | | | | | | |
| T148A | 8 | 383 ± 109 | 435 ± 128 | 10.2 ± 2.1 | 1620 ± 195 | 1747 ± 234 | 317 ± 82 | 0.58 ± 0.08 |
| Catalyst Biosciences WT | 2 | 329 | 360 | 11.9 | 2036 | 2121 | 229 | 0.48 |
| A103N/N105S | 2 | 375 | 481 | 12.5 | 2841 | 3068 | 177 | 0.33 |
| D104N/K106S | 2 | 428 | 558 | 13.9 | 3379 | 3786 | 164 | 0.26 |
| K106N/V108S | 2 | 510 | 629 | 12.8 | 2748 | 3202 | 234 | 0.32 |
| D85N | 2 | 528 | 607 | 9.5 | 1798 | 2046 | 372 | 0.49 |
| D64N | 2 | 447 | 519 | 11.8 | 1933 | 2152 | 304 | 0.47 |
| D64A | 2 | 364 | 372 | 11.5 | 1351 | 1466 | 359 | 0.68 |
| N167D | 2 | 334 | 318 | 8.9 | 1129 | 1176 | 410 | 0.85 |
| N167Q | 3 | 337 ± 8.8 | 323 ± 4.2 | 8.2 ± 1.2 | 1495 ± 258 | 1554 ± 268 | 318 ± 42 | 0.66 ± 0.10 |
| S61A | 2 | 397 | 412 | 10.0 | 1685 | 1800 | 325 | 0.57 |
| S53A | 2 | 382 | 462 | 11.2 | 2146 | 2321 | 238 | 0.43 |
| T159A | 2 | 232 | 227 | 10.5 | 1036 | 1048 | 315 | 0.97 |
| T169A | 2 | 348 | 319 | 8.3 | 836 | 889 | 567 | 1.15 |
| T172A | 3 | 494 ± 187 | 571 ± 214 | 11.2 ± 2.9 | 2055 ± 408 | 2366 ± 676 | 295 ± 31 | 0.45 ± 0.13 |
| T179A | 2 | 377 | 431 | 12.5 | 2291 | 2458 | 223 | 0.42 |
| Y155H | 2 | 465 | 552 | 11.6 | 2365 | 2638 | 253 | 0.38 |
| Y155Q | 1 | 552 | 645 | 13.6 | 2583 | 3045 | 262 | 0.33 |
| S158E | 2 | 433 | 471 | 14.5 | 2029 | 2222 | 291 | 0.46 |
| N157Q | 2 | 335 | 352 | 11.3 | 1185 | 1238 | 395 | 0.83 |
| N157D | 2 | 290 | 265 | 9.9 | 1166 | 1211 | 393 | 0.93 |
| Y155F | 2 | 443 | 567 | 18.1 | 3941 | 4375 | 149 | 0.23 |
| A103N/N105S/Y155F | 2 | 562 | 619 | 13.1 | 2427 | 2496 | 325 | 0.40 |
| D104N/K106S/Y155F | 3 | 514 ± 80 | 581 ± 81 | 13.8 ± 1.0 | 3057 ± 1032 | 3181 ± 989 | 243 ± 47 | 0.34 ± 0.13 |
| D203N/F205T | 3 | 481 ± 69 | 566 ± 29 | 9.4 ± 1.9 | 2028 ± 448 | 2289 ± 489 | 314 ± 91 | 0.45 ± 0.09 |
| D203N/F205T/D85N | 1 | 291 | 406 | 12.4 | 1538 | 2044 | 205 | 0.49 |
| K228N/D85N | 2 | 459 | 565 | 11.3 | 2616 | 2926 | 227 | 0.35 |
| K228N/A103N/N105S | 2 | 583 | 701 | 14.4 | 3032 | 3301 | 255 | 0.30 |
| K228N/D104N/K106S | 2 | 801 | 913 | 13.6 | 2050 | 2238 | 513 | 0.45 |
| K228N/Y155F | 2 | 626 | 679 | 8.6 | 2073 | 2149 | 431 | 0.47 |
| K228N/D104N/K106S/Y155F | 2 | 551 | 614 | 14.0 | 3730 | 3822 | 211 | 0.27 |
| I251S | 2 | 565 | 718 | 10.1 | 2646 | 3137 | 260 | 0.32 |
| I251S/A103N/N105S | 2 | 444 | 542 | 14.3 | 2445 | 2719 | 241 | 0.38 |
| I251S/D104N/K106S | 2 | 692 | 802 | 13.9 | 2533 | 2664 | 375 | 0.38 |
| I251S/Y155F | 2 | 572 | 660 | 12.2 | 2591 | 2790 | 291 | 0.37 |
| A262S | 3 | 373 ± 87 | 453 ± 91 | 14.4 ± 3.8 | 2716 ± 732 | 2926 ± 908 | 188 ± 29 | 0.36 ± 0.10 |
| E410N* | 2 | 439 | 551 | 7.4 | 893 | 1365 | 469 | 0.75 |
| E239N | 2 | 338 | 416 | 10.7 | 1657 | 1908 | 257 | 0.54 |
| K247N/N249S | 6 | 627 ± 174 | 734 ± 244 | 10.8 ± 3.4 | 2196 ± 737 | 2545 ± 795 | 387 ± 154 | 0.42 ± 0.11 |
| Y155F/K247N/N249S | 2 | 538 | 608 | 10.6 | 1752 | 1880 | 420 | 0.53 |
| K247N/N249S/A103N/N105S | 2 | 736 | 852 | 21.5 | 4369 | 4699 | 226 | 0.21 |
| K247N/N249S/D104N/K106S/ | 2 | 603 | 714 | 16.8 | 3744 | 3889 | 233 | 0.27 |
| Y155F | | | | | | | | |
| S319N/L321S | 2 | 351 | 427 | 11.4 | 2270 | 2409 | 210 | 0.42 |
| N260S | 3 | 496 ± 157 | 619 ± 170 | 11.5 ± 3.8 | 3364 ± 1300 | 3687 ± 1457 | 231 ± 156 | 0.30 ± 0.11 |
| D104N/K106S/N260S | 2 | 805 | 1001 | 16.1 | 4736 | 5248 | 220 | 0.20 |
| Y155F/N260S | 2 | 607 | 682 | 18.4 | 3408 | 3530 | 257 | 0.27 |
| Y284N | 2 | 400 | 478 | 9.0 | 2052 | 2210 | 270 | 0.46 |
| R318Y/E410N | 1 | 428 | 474 | 6.1 | 575 | 686 | 900 | 1.46 |
| R338E/E410N | 2 | 334 | 376 | 6.2 | 718 | 844 | 570 | 1.18 |
| R338E/R403E/E4100N | 5 | 436 ± 24 | 507 ± 29 | 13.4 ± 2.0 | 3052 ± 522 | 3302 ± 656 | 196 ± 49 | 0.31 ± 0.06 |
| D203N/F205T/E240N | 2 | 600 | 679 | 6.8 | 671 | 799 | 1080 | 1.25 |
| D203N/F205T/R338E | 2 | 307 | 419 | 9.3 | 1186 | 1586 | 281 | 0.63 |
| D203N/F205T/R338A | 2 | 317 | 403 | 9.0 | 1063 | 1397 | 327 | 0.72 |
| D203N/F205T/R318Y | 2 | 258 | 286 | 8.7 | 508 | 601 | 732 | 1.91 |
| D203N/F205T/R338E/R403E | 2 | 303 | 419 | 11.3 | 2105 | 2804 | 156 | 0.36 |
| K228N/E410N | 2 | 373 | 479 | 6.0 | 721 | 1025 | 522 | 0.98 |
| K228N/R338E | 2 | 248 | 340 | 10.4 | 1403 | 1736 | 207 | 0.58 |
| R318Y/R338E/E410N | 5 | 424 ± 306 | 515 ± 378 | 5.8 ± 1.6 | 645 ± 310 | 774 ± 454 | 778 ± 272 | 1.6 ± 0.73 |
| R318Y/R338E/E410N/D104N/ | 2 | 502 | 531 | 8.9 | 2008 | 2041 | 355 | 0.49 |
| K106S | | | | | | | | |
| R318Y/R338E/E410N/Y155F | 2 | 555 | 584 | 6.5 | 678 | 721 | 1136 | 1.53 |
| K228N/R318Y/E410N | 1 | 304 | 408 | 6.0 | 686 | 906 | 485 | 1.10 |
| R318Y/R338E/R403E/E410N | 5 | 442 ± 22 | 534 ± 28 | 16.4 ± 3.7 | 3902 ± 867 | 4232 ± 996 | 157 ± 38 | 0.25 ± 0.05 |
| A103N/N105S/R318Y/R338E/ | 2 | 421 | 527 | 16.2 | 3605 | 3935 | 157 | 0.26 |
| R403E/E410N | | | | | | | | |
| D104N/K106S/R318Y/R338E/ | 2 | 417 | 517 | 15.1 | 3114 | 3392 | 183 | 0.30 |
| R403E/E410N | | | | | | | | |
| Y155F/R318Y/R338E/R403E/ | 2 | 565 | 649 | 12.4 | 3687 | 3772 | 226 | 0.27 |
| E410N | | | | | | | | |
| R318Y/R338E/R403E/E410N/ | 3 | 669 ± 145 | 819 ± 223 | 17.2 ± 2.0 | 5844 ± 1064 | 6204 ± 1393 | 156 ± 8.7 | 0.17 ± 0.04 |
| A103N/N105S/Y155F | | | | | | | | |
| R318Y/R338E/R403E/E410N/ | 2 | 472 | 575 | 14.4 | 5885 | 5967 | 114 | 0.17 |
| D104N/K106S/Y155F | | | | | | | | |
| D203N/F205T/R318Y/E410N | 1 | 431 | 475 | 8.0 | 637 | 761 | 816 | 1.31 |
| R338L | 2 | 368 | 377 | 11.2 | 1761 | 1861 | 285 | 0.54 |
| K316M | 2 | 527 | 665 | 7.9 | 1846 | 2142 | 356 | 0.47 |
| E239S | 2 | 462 | 542 | 11.3 | 2184 | 2416 | 278 | 0.41 |
| E239A | 2 | 538 | 544 | 13.1 | 1973 | 2209 | 353 | 0.45 |
| E239R | 2 | 431 | 709 | 8.9 | 1668 | 2020 | 307 | 0.50 |
| E239K | 2 | 400 | 370 | 14.4 | 2107 | 2222 | 278 | 0.48 |
| H257F | 2 | 328 | 357 | 10.3 | 1689 | 1820 | 273 | 0.70 |
| H257Y | 2 | 352 | 353 | 13.6 | 1971 | 2063 | 245 | 0.49 |
| H257E | 2 | 491 | 520 | 10.9 | 2185 | 2411 | 294 | 0.42 |
| H257S | 2 | 435 | 511 | 8.2 | 1630 | 1769 | 358 | 0.57 |
| T412A | 2 | 473 | 539 | 7.1 | 1561 | 1756 | 379 | 0.58 |
| T412V | 2 | 579 | 665 | 8.3 | 1258 | 1454 | 565 | 0.69 |
| E410N/T412A | 2 | 461 | 514 | 2.8 | 364 | 398 | 1679 | 2.51 |
| E410N/T412V | 2 | 340 | 390 | 3.7 | 431 | 487 | 906 | 2.27 |
| E410Q | 2 | 276 | 283 | 7.2 | 445 | 484 | 836 | 2.19 |
| E410S | 2 | 310 | 286 | 7.2 | 753 | 775 | 587 | 1.32 |
| E410A | 2 | 363 | 328 | 8.6 | 528 | 554 | 946 | 1.81 |
| E410D | 2 | 348 | 377 | 9.2 | 1473 | 1596 | 320 | 0.63 |
| N346D | 2 | 349 | 395 | 13.3 | 2817 | 2956 | 170 | 0.34 |
| Y155F/N346D | 2 | 472 | 478 | 17.0 | 3934 | 3986 | 176 | 0.26 |
| N346Y | 2 | 329 | 325 | 11.7 | 1246 | 1297 | 365 | 0.77 |
| Y345T | 2 | 359 | 453 | 6.1 | 1124 | 1200 | 438 | 0.85 |
| T343R | 2 | 402 | 504 | 6.5 | 1143 | 1234 | 487 | 0.85 |
| T343E | 2 | 414 | 461 | 12.6 | 1740 | 1877 | 318 | 0.53 |
| T343Q | 2 | 434 | 442 | 9.0 | 1626 | 1737 | 408 | 0.63 |
| F342I | 2 | 400 | 476 | 8.3 | 1133 | 1224 | 491 | 0.88 |
| T343R/Y345T | 2 | 325 | 324 | 9.1 | 1094 | 1130 | 422 | 0.90 |
| R318Y/R338E | 2 | 340 | 313 | 11.2 | 1402 | 1452 | 336 | 0.69 |
| K228N/I251S | 2 | 586 | 657 | 11.3 | 1473 | 1588 | 551 | 0.65 |
| K228N/R318Y/R338E/R403E/ | 2 | 476 | 647 | 9.1 | 2400 | 2726 | 261 | 0.37 |
| E410N | | | | | | | | |
| K228N/R318Y/R338E/R403E/ | 3 | 615 ± 135 | 750 ± 191 | 18.6 ± 2.1 | 5496 ± 2044 | 5970 ± 2260 | 158 ± 50 | 0.18 ± 0.06 |
| E410N/Y155F | | | | | | | | |
| K228N/R318Y/R338E/R403E/ | 2 | 587 | 713 | 24.8 | 6153 | 6725 | 125 | 0.15 |
| E410N/D85N | | | | | | | | |
| I251S/R318Y/R338E/R403E/ | 3 | 412 ± 140 | 542 ± 181 | 15.7 ± 4.9 | 2306 ± 884 | 2636 ± 1261 | 242 ± 89 | 0.44 ± 0.17 |
| E410N | | | | | | | | |
| D104N/K106S/I251S/R318Y/ | 4 | 687 ± 60 | 874 ± 82 | 17.2 ± 2.2 | 7653 ± 456 | 8127 ± 520 | 122 ± 10 | 0.12 ± 0.01 |
| R338E/R403E/E410N | | | | | | | | |
| I251S/R318Y/R338E/R403E/ | 2 | 492 | 620 | 19.9 | 5704 | 6510 | 110 | 0.15 |
| E410N/Y155F | | | | | | | | |
| I251S/R318Y/R338E/E410N | 2 | 591 | 630 | 7.5 | 1245 | 1292 | 664 | 0.78 |
| D104N/K106S/D104N/K106S/ | 2 | 726 | 819 | 16.4 | 1512 | 1612 | 650 | 0.62 |
| I251S/R318Y/R338E/E410N/ | | | | | | | | |
| K247N/N249S/R318Y/R338E/ | 2 | 637 | 807 | 15.4 | 5283 | 5541 | 170 | 0.18 |
| R403E/E410N | | | | | | | | |
| Y155F/K247N/N249S/R318Y/ | 2 | 613 | 758 | 13.8 | 5335 | 5549 | 160 | 0.18 |
| R338E/R403E/E410N | | | | | | | | |
| A103N/N105S/K247N/N249S/ | 2 | 615 | 783 | 18.6 | 7319 | 7612 | 117 | 0.13 |
| R318Y/R338E/R403E/E410N | | | | | | | | |
| D104N/K106S/K247N/N249S/ | 2 | 626 | 754 | 19.4 | 6332 | 6580 | 140 | 0.15 |
| R318Y/R338E/R403E/E410N | | | | | | | | |
| K228N/N84S/R318Y/R338E/ | 2 | 512 | 539 | 18.1 | 1925 | 1967 | 396 | 0.54 |
| E410N | | | | | | | | |
| Y155F/K228N/N84S/R318Y/ | 2 | 617 | 685 | 8.1 | 1170 | 1221 | 745 | 0.83 |
| R338E/E410N | | | | | | | | |
| R318Y/R338E/R403E/E410S | 2 | 382 | 395 | 14.7 | 2897 | 2971 | 184 | 0.34 |
| R318Y/R338E/E410S | 2 | 356 | 326 | 7.7 | 488 | 511 | 1066 | 2.08 |
| K228N/K247N/N249S | 2 | 662 | 753 | 19.6 | 3390 | 3578 | 268 | 0.28 |
| K228N/K247N/N249S/D104N/ | 3 | 781 ± 55 | 939 ± 48 | 18.5 ± 3.8 | 6111 ± 1900 | 6606 ± 1949 | 183 ± 63 | 0.16 ± 0.04 |
| K106S/Y155F | | | | | | | | |
| K228N/K247N/N249S/D104N/ | 2 | 758 | 838 | 17.9 | 3792 | 4035 | 271 | 0.25 |
| K106S | | | | | | | | |
| K228N/K247N/N249S/Y155F | 2 | 549 | 643 | 17.2 | 3002 | 3269 | 246 | 0.31 |
| I251S/R318Y/R338E/R403E/ | 3 | 599 ± 89 | 753 ± 121 | 21.7 ± 3.2 | 8567 ± 2834 | 9233 ± 2860 | 96.6 ± 15.4 | 0.11 ± 0.03 |
| E410N/Y155F | | | | | | | | |
| R318Y/R338E/R403E/E410N/ | 2 | 424 | 456 | 20.0 | 4730 | 4892 | 124 | 0.20 |
| T412V | | | | | | | | |
| R318Y/R338E/R403E/E410N/ | 2 | 380 | 439 | 17.5 | 4994 | 5115 | 107 | 0.20 |
| T412A | | | | | | | | |
| R318Y/R338E/R403E/T412A | 3 | 399 ± 88 | 477 ± 108 | 19.7 ± 0.7 | 4320 ± 2385 | 4505 ± 2357 | 144 ± 48 | 0.27 ± 0.15 |
| R318Y/R3380E/T412A | 2 | 462 | 401 | 13.6 | 1674 | 1691 | 398 | 0.60 |
| N260S/R318Y/R338E/R403E/ | 2 | 583 | 743 | 23.9 | 6821 | 7488 | 111 | 0.13 |
| E410N | | | | | | | | |
| D104N/K106S/N260S/R318Y/ | 2 | 779 | 999 | 17.2 | 7100 | 7728 | 145 | 0.12 |
| R338E/R403E/E410N | | | | | | | | |
| Y155F/N260S/R318Y/R338E/ | 2 | 628 | 758 | 21.4 | 5214 | 5465 | 167 | 0.21 |
| R403E/E410N | | | | | | | | |
| R318Y/R338E/N346D/R403E/ | 2 | 474 | 575 | 25.2 | 7623 | 8140 | 86 | 0.12 |
| E410N | | | | | | | | |
| Y155F/R318Y/R338E/N346D/ | 2 | 540 | 641 | 18.2 | 5039 | 5172 | 154 | 0.20 |
| R403E/E410N | | | | | | | | |
| K247N/N249S/N260S | 2 | 549 | 632 | 17.4 | 4156 | 4262 | 186 | 0.23 |
| Y155F/K247N/N249S/N260S | 2 | 691 | 814 | 24.0 | 3857 | 4085 | 244 | 0.22 |
| D104N/K106S/K247N/N249S/ | 2 | 712 | 859 | 16.5 | 4187 | 4458 | 235 | 0.23 |
| N260S | | | | | | | | |
| D104N/K106S/Y155F/K247N/ | 2 | 680 | 856 | 23.3 | 7026 | 7423 | 134 | 0.14 |
| N249S/N260S | | | | | | | | |
| K247N/N249S/N260S/R318Y/ | 2 | 691 | 875 | 18.9 | 6353 | 6737 | 149 | 0.13 |
| R338E/R403E/E410N | | | | | | | | |
| R318Y/R338E/T343R/R403E/ | 2 | 531 | 560 | 20.5 | 3766 | 3862 | 200 | 0.27 |
| E410N | | | | | | | | |
| R338E/T343R | 2 | 534 | 453 | 12.8 | 798 | 813 | 949 | 1.23 |

*80% glycosylated form of E410N

TABLE 36
PK properties of FIX variants assessed by ELISA

| Mutation | Mutation | | Terminal | AUC/Dose | AUC/Dose | MRT | | | |
|---|---|---|---|---|---|---|---|---|---|
| (Mature FIX Numbering) | (Chymotrypsin Numbering) | N | T½ | (0-last) | (0-inf) | (0-inf) | Cmax/Dose | Vz | Cl |
| N157D | N[157]D | 2 | 290 | 1166 | 1211 | 265 | 9.9 | 393 | 0.93 |
| Y155F | Y[155]F | 2 | 443 | 3941 | 4375 | 567 | 18.1 | 149 | 0.23 |
| A103N/N105S/Y155F | A[103]N/N[105]S/Y[155]F | 2 | 562 | 2427 | 2496 | 619 | 13.1 | 325 | 0.40 |
| D104N/K106S/Y155F | D[104]N/K[106]S/Y[155]F | 3 | 514 ± 79.8 | 3060 ± 1030 | 3180 ± 989 | 581 ± 81.0 | 13.8 ± 1.02 | 243 ± 47.4 | 0.341 ± 0.128 |
| WT | Catalyst Biosciences WT | 2 | 329 | 2036 | 2121 | 360 | 11.9 | 229 | 0.48 |
| A103N/N105S | A[103]N/N[105]S | 2 | 375 | 2841 | 3068 | 481 | 12.5 | 177 | 0.33 |
| D104N/K106S | D[104]N/K[106]S | 2 | 428 | 3379 | 3786 | 558 | 13.9 | 164 | 0.26 |
| K106N/V108S | K[106]N/V[108]S | 2 | 510 | 2748 | 3202 | 629 | 12.8 | 234 | 0.32 |
| D85N | D[85]N | 4 | 575 ± 89.3 | 1530 ± 321 | 1680 ± 83.3 | 623 ± 83.3 | 9.10 ± 0.518 | 528 ± 184 | 0.619 ± 0.156 |
| T148A | BeneFIX, T[148]A | 3 | 314 ± 128 | 1300 ± 298 | 1520 ± 158 | 366 ± 105 | 9.12 ± 1.52 | 308 ± 160 | 0.662 ± 0.071 |
| T148A | T[148]A | 8 | 383 ± 109 | 1620 ± 195 | 1750 ± 234 | 435 ± 128 | 10.2 ± 2.09 | 317 ± 82.3 | 0.582 ± 0.084 |
| K5A | K[5]A | 2 | 271 | 1548 | 1583 | 311 | 10.5 | 251 | 0.64 |
| D64N | D[64]N | 2 | 447 | 1933 | 2152 | 519 | 11.8 | 304 | 0.47 |
| D64A | D[64]A | 2 | 364 | 1351 | 1466 | 372 | 11.5 | 359 | 0.68 |
| N167D | N[167]D | 2 | 334 | 1129 | 1176 | 318 | 8.9 | 410 | 0.85 |
| N167Q | N[167]Q | 3 | 337 ± 8.75 | 1500 ± 258 | 1550 ± 268 | 323 ± 4.25 | 8.20 ± 1.17 | 318 ± 42.5 | 0.655 ± 0.103 |
| S61A | S[61]A | 2 | 397 | 1685 | 1800 | 412 | 10.0 | 325 | 0.57 |
| S53A | S[53]A | 2 | 382 | 2146 | 2321 | 462 | 11.2 | 238 | 0.43 |
| T159A | T[159]A | 2 | 232 | 1036 | 1048 | 227 | 10.5 | 315 | 0.97 |
| T169A | T[169]A | 2 | 348 | 836 | 889 | 319 | 8.3 | 567 | 1.15 |
| T172A | T[172]A | 3 | 494 ± 187 | 2050 ± 408 | 237 ± 676 | 571 ± 214 | 11.2 ± 2.89 | 295 ± 31.5 | 0.447 ± 0.132 |
| T179A | T[179]A | 2 | 377 | 2291 | 2458 | 431 | 12.5 | 223 | 0.42 |
| Y155H | Y[155]H | 2 | 465 | 2365 | 2638 | 552 | 11.6 | 253 | 0.38 |
| Y155Q | Y[155]Q | 1 | 552 | 2583 | 3045 | 645 | 13.6 | 262 | 0.33 |
| S158E | S[158]E | 2 | 433 | 2029 | 2222 | 471 | 14.5 | 291 | 0.46 |
| N157Q | N[157]Q | 2 | 335 | 1185 | 1238 | 352 | 11.3 | 395 | 0.83 |
| D203N/F205T | D39N/F41T | 3 | 481 ± 69.0 | 2030 ± 448 | 2290 ± 489 | 566 ± 28.6 | 9.43 ± 1.93 | 314 ± 91.3 | 0.449 ± 0.087 |
| D85N/D203N/F205T | D[85]N/D39N/F41T | 1 | 291 | 1538 | 2044 | 406 | 12.4 | 205 | 0.49 |
| K228N | K63N | 3 | 490 ± 57.8 | 2340 ± 519 | 2570 ± 682 | 570 ± 27.9 | 12.3 ± 1.58 | 296 ± 119 | 0.410 ± 0.118 |
| A103N/N105S/K228N | A[103]N/N[105]S/K63N | 2 | 583 | 3032 | 3301 | 701 | 14.4 | 255 | 0.30 |
| D104N/K106S/K228N | D[104]N/K[106]S/K63N | 2 | 801 | 2050 | 2238 | 913 | 13.6 | 513 | 0.45 |
| Y155F/K228N | Y[155]F/K63N | 2 | 626 | 2073 | 2149 | 679 | 8.6 | 431 | 0.47 |
| D104N/K106S/Y155F/K228N | D[104]N/K[106]S/Y[155]F/K63N | 2 | 551 | 3730 | 3822 | 614 | 14.0 | 211 | 0.27 |
| I251S | I86S | 2 | 565 | 2646 | 3137 | 718 | 10.1 | 260 | 0.32 |
| A103N/N105S/I251S | A[103]N/N[105]S/I86S | 2 | 444 | 2445 | 2719 | 542 | 14.3 | 241 | 0.38 |
| D104N/K106S/I251S | D[104]N/K[106]S/I86S | 2 | 692 | 2533 | 2664 | 802 | 13.9 | 375 | 0.38 |
| Y155F/I251S | Y[155]F/I86S | 2 | 572 | 2591 | 2790 | 660 | 12.2 | 291 | 0.37 |
| A262S | A95bS | 2 | 373 | 2716 | 2926 | 453 | 14.4 | 188 | 0.36 |
| E410N | E240N | 2 | 439 | 893 | 1365 | 551 | 7.4 | 469 | 0.75 |
| E239N | E74N | 2 | 338 | 1657 | 1908 | 416 | 10.7 | 257 | 0.54 |
| K247N/N249S | K82N/N84S | 6 | 627 ± 174 | 2200 ± 737 | 2540 ± 795 | 734 ± 244 | 10.8 ± 3.41 | 387 ± 154 | 0.420 ± 0.106 |
| Y155F/K247N/N249S | Y[155]F/K82N/N84S | 2 | 538 | 1752 | 1880 | 608 | 10.6 | 420 | 0.53 |
| A103N/N105S/K247N/N249S | A[103]N/N[105]S/K82N/N84S | 2 | 736 | 4369 | 4699 | 852 | 21.5 | 226 | 0.21 |
| D104N/K106S/K247N/N249S | D[104]N/K[106]S/K82N/N84S | 2 | 571 | 2052 | 2109 | 632 | 16.2 | 426 | 0.51 |
| D104N/K106S/Y155F/K247N/N249S | D[104]N/K[106]S/Y[155]F/K82N/N84S | 2 | 603 | 3744 | 3889 | 714 | 16.8 | 233 | 0.27 |
| S319N/L321S | S151N/L153S | 2 | 351 | 2270 | 2409 | 427 | 11.4 | 210 | 0.42 |
| N260S | N95S | 3 | 496 ± 157 | 3360 ± 1300 | 3690 ± 1460 | 619 ± 170 | 11.5 ± 3.18 | 231 ± 156 | 0.295 ± 0.105 |
| D104N/K106S/N260S | D[104]N/K[106]S/N95S | 2 | 805 | 4736 | 5248 | 1001 | 16.1 | 220 | 0.20 |
| Y155F/N260S | Y[155]F/N95S | 2 | 607 | 3408 | 3530 | 682 | 18.4 | 257 | 0.27 |
| Y284N | Y117N | 2 | 400 | 2052 | 2210 | 478 | 9.0 | 270 | 0.46 |
| R318Y/E410N | R150Y/E240N | 1 | 428 | 575 | 686 | 474 | 6.1 | 900 | 1.46 |
| R338E/E410N | R170E/E240N | 2 | 334 | 718 | 844 | 376 | 6.2 | 570 | 1.18 |
| R338E/R403E/E410N | R170E/R233E/E240N | 5 | 436 ± 24.4 | 3050 ± 522 | 3300 ± 656 | 507 ± 28.9 | 13.4 ± 2.03 | 196 ± 49.2 | 0.312 ± 0.063 |
| D203N/F205T/E410N | D39N/F41T/E240N | 2 | 600 | 671 | 799 | 679 | 6.8 | 1080 | 1.25 |
| D203N/F205T/R338E | D39N/F41T/R170E | 2 | 307 | 1186 | 1586 | 419 | 9.3 | 281 | 0.63 |
| D203N/F205T/R338A | D39N/F41T/R170A | 2 | 317 | 1063 | 1397 | 403 | 9.0 | 327 | 0.72 |
| D203N/F205T/R318Y | D39N/F41T/R150Y | 2 | 258 | 508 | 601 | 286 | 8.7 | 732 | 1.91 |
| D203N/F205T/R338E/R403E | D39N/F41T/R170E/R233E | 2 | 303 | 2105 | 2804 | 419 | 11.3 | 156 | 0.36 |
| K228N/E410N | K63N/E240N | 2 | 373 | 721 | 1025 | 479 | 6.0 | 522 | 0.98 |
| K228N/R338E | K63N/R170E | 2 | 248 | 1403 | 1736 | 340 | 10.4 | 207 | 0.58 |
| R318Y/R338E/E410N | R150Y/E240N/R170E | 5 | 424 ± 306 | 645 ± 310 | 774 ± 454 | 515 ± 378 | 5.78 ± 1.56 | 778 ± 272 | 1.62 ± 0.730 |
| D104N/K106S/R318Y/R338E/E410N | D[104]N/K[106]S/R150Y/R170E/E240N/ | 2 | 502 | 2008 | 2041 | 531 | 8.9 | 355 | 0.49 |
| Y155F/R318Y/R338E/E410N | Y[155]F/R150Y/R170E/E240N | 2 | 555 | 678 | 721 | 584 | 6.5 | 1136 | 1.53 |
| K228N/R318Y/E410N | K63N/R150Y/E240N | 1 | 304 | 686 | 906 | 408 | 6.0 | 485 | 1.10 |
| R318Y/R338E/R403E/E410N | R150Y/R170E/R233E/E240N | 5 | 442 ± 22.1 | 3900 ± 867 | 4230 ± 996 | 534 ± 28.0 | 16.4 ± 3.72 | 157 ± 38.3 | 0.246 ± 0.051 |
| A103N/N105S/R318Y/R338E/R403E/ | A[103]N/N[105]S/R150Y/R170E/R233E/ | 2 | 421 | 3605 | 3935 | 527 | 16.2 | 157 | 0.26 |
| E410N | E240N | | | | | | | | |
| D104N/K106S/R318Y/R338E/R403E/ | D[104]N/K[106]S/R150Y/R170E/R233E/ | 2 | 417 | 3114 | 3392 | 517 | 15.1 | 183 | 0.30 |
| E410N | E240N | | | | | | | | |
| Y155F/R318Y/R338E/R403E/E410N | Y[155]F/R150Y/R170E/R233E/E240N | 2 | 565 | 3687 | 3772 | 649 | 12.4 | 226 | 0.27 |
| A103N/N105S/Y155F/R318Y/R338E/ | A[103]N/N[105]S/Y[155]F/ | 3 | 669 ± 145 | 5840 ± 1060 | 6200 ± 1390 | 819 ± 223 | 17.2 ± 2.02 | 156 ± 8.74 | 0.167 ± 0.039 |
| R403E/E410N | R150Y/R170E/R233E/E240N | | | | | | | | |
| D104N/K106S/Y155F/R318Y/R338E/ | D[104]N/K[106]S/Y[155]F/ | 2 | 472 | 5885 | 5967 | 575 | 14.4 | 114 | 0.17 |
| R403E/E410N | R150Y/R170E/R233E/E240N | | | | | | | | |
| D203N/F205T/R318Y/E410N | D39N/F41T/R150Y/E240N | 1 | 431 | 637 | 761 | 475 | 8.0 | 816 | 1.31 |
| R338L | R170L | 2 | 368 | 1761 | 1861 | 377 | 11.2 | 285 | 0.54 |
| K316M | K148M | 2 | 527 | 1846 | 2142 | 665 | 7.9 | 356 | 0.47 |
| E239S | E74S | 2 | 462 | 2184 | 2416 | 542 | 11.3 | 278 | 0.41 |
| E239A | E74A | 2 | 538 | 1973 | 2209 | 544 | 13.1 | 353 | 0.45 |
| E239R | E74R | 2 | 431 | 1668 | 2020 | 709 | 8.9 | 307 | 0.50 |
| E239K | E74K | 2 | 400 | 2107 | 2222 | 370 | 14.4 | 278 | 0.48 |
| H257F | H92F | 2 | 328 | 1689 | 1820 | 357 | 10.3 | 273 | 0.70 |
| H257Y | H92Y | 2 | 352 | 1971 | 2063 | 353 | 13.6 | 245 | 0.49 |
| H257E | H92E | 2 | 491 | 2185 | 2411 | 520 | 10.9 | 294 | 0.42 |
| H257S | H92S | 2 | 435 | 1630 | 1769 | 511 | 8.2 | 358 | 0.57 |
| T412A | T242A | 2 | 473 | 1561 | 1756 | 539 | 7.1 | 379 | 0.58 |
| T412V | T242V | 2 | 579 | 1258 | 1454 | 665 | 8.3 | 565 | 0.69 |
| E410N/T412A | E240N/T242A | 2 | 461 | 364 | 398 | 514 | 2.8 | 1679 | 2.51 |
| E410N/T412V | E240N/T242V | 2 | 340 | 431 | 487 | 390 | 3.7 | 906 | 2.27 |
| E410Q | E240Q | 2 | 276 | 445 | 484 | 283 | 7.2 | 836 | 2.19 |
| E410S | E240S | 2 | 310 | 753 | 775 | 286 | 7.2 | 587 | 1.32 |
| E410A | E240A | 2 | 363 | 528 | 554 | 328 | 8.6 | 946 | 1.81 |
| E410D | E240D | 2 | 348 | 1473 | 1596 | 377 | 9.2 | 320 | 0.63 |
| N346D | N178D | 2 | 349 | 2817 | 2956 | 395 | 13.3 | 170 | 0.34 |
| Y155F/N346D | 178D/Y[155]F | 2 | 472 | 3934 | 3986 | 478 | 17.0 | 176 | 0.26 |
| N346Y | N178Y | 2 | 329 | 1246 | 1297 | 325 | 11.7 | 365 | 0.77 |
| Y345T | Y177T | 2 | 359 | 1124 | 1200 | 453 | 6.1 | 438 | 0.85 |
| T343R | Y175R | 2 | 402 | 1143 | 1234 | 504 | 6.5 | 487 | 0.85 |
| T343E | T175E | 2 | 414 | 1740 | 1877 | 461 | 12.6 | 318 | 0.53 |
| T343Q | Y175Q | 2 | 434 | 1626 | 1737 | 442 | 9.0 | 408 | 0.63 |
| F342I | F174I | 2 | 400 | 1133 | 1224 | 476 | 8.3 | 491 | 0.88 |
| T343R/Y345T | T175R/Y177T | 2 | 325 | 1094 | 1130 | 324 | 9.1 | 422 | 0.90 |
| R318Y/R338E | R150Y/R170E | 2 | 340 | 1402 | 1452 | 313 | 11.2 | 336 | 0.69 |
| K228N/I251S | K63N/I86S | 2 | 586 | 1473 | 1588 | 657 | 11.3 | 551 | 0.65 |
| K228N/R318Y/R338E/R403E/E410N | K63N/R150Y/R170E/R233E/E240N | 2 | 476 | 2400 | 2726 | 647 | 9.1 | 261 | 0.37 |
| Y155F/K228N/R318Y/R338E/R403E/ | Y[155]F/K63N/R150Y/R170E/R233E/E240N | 3 | 615 ± 135 | 5500 ± 2040 | 5970 ± 2260 | 750 ± 191 | 18.6 ± 2.14 | 158 ± 50.1 | 0.183 ± 0.062 |
| E410N | | | | | | | | | |
| D85N/K228N/R318Y/R338E/R403E/ | D[85]N/K63N/R150Y/R170E/R233E/E240N | 2 | 587 | 6153 | 6725 | 713 | 24.8 | 125 | 0.15 |
| E410N | | | | | | | | | |
| I251S/R318Y/R338E/R403E/E410N | I86S/R150Y/R170E/R233E/E240N | 3 | 412 ± 140 | 2310 ± 884 | 2640 ± 1260 | 542 ± 181 | 15.7 ± 4.89 | 242 ± 89.4 | 0.438 ± 0.171 |
| D104N/K106S/I251S/R318Y/R338E/ | D[104]N/K[106]S/I86S/R150Y/ | 4 | 687 ± 60.2 | 7650 ± 456 | 8130 ± 520 | 874 ± 81.7 | 17.2 ± 2.24 | 122 ± 10.1 | 0.123 ± 0.008 |
| R403E/E410N | R170E/R233E/E240N | | | | | | | | |
| Y155F/I251S/R318Y/R338E/R403E/ | Y[155]F/I86S/R150Y/R170E/R233E/E240N | 2 | 492 | 5704 | 6510 | 620 | 19.9 | 110 | 0.15 |
| E410N | | | | | | | | | |
| I251S/R318Y/R338E/E410N | I86S/R150Y/R170E/E240N | 2 | 591 | 1245 | 1292 | 630 | 7.5 | 664 | 0.78 |
| D104N/K106S/I251S/R318Y/R338E/ | D[104]N/K[106]S I86S/R150Y/ | 2 | 726 | 1512 | 1612 | 819 | 16.4 | 650 | 0.62 |
| E410N | R170E/E240N | | | | | | | | |
| K247N/N249S/R318Y/R338E/R403E/ | K82N/N84S/R150Y/R170E/R233E/E240N | 2 | 637 | 5283 | 5541 | 807 | 15.4 | 170 | 0.18 |
| E410N | | | | | | | | | |
| Y155F/K247N/N249S/R318Y/R338E/ | Y[155]F/K82N/N84S/R150Y/R170E/R233E/ | 2 | 613 | 5335 | 5549 | 758 | 13.8 | 160 | 0.18 |
| R403E/E410N | E240N | | | | | | | | |
| A103N/N105S/K247N/N249S/R318Y/ | A[103]N/N[105]S K82N/N84S/ | 2 | 615 | 7319 | 7612 | 783 | 18.6 | 117 | 0.13 |
| R338E/R403E/E410N | R150Y/R170E/R233E/E240N/ | | | | | | | | |
| D104N/K106S/K247N/N249S/R318Y/ | D[104]N/K[106]S K82N/N84S/ | 2 | 626 | 6332 | 6580 | 754 | 19.4 | 140 | 0.15 |
| R338E/R403E/E410N | R150Y/R170E/R233E/E240N/ | | | | | | | | |
| D104N/K106S/Y155F/K247N/N249S/ | D[104]N/K[106]S/Y[155]F/ | 2 | 846 | 8069 | 8807 | 1020 | 18.4 | 139 | 0.11 |
| R318Y/R338E/R403E/E410N | K82N/N84S/R150Y/R170E/R233E/E240N | | | | | | | | |
| K247N/N249S/R318Y/R338E/E410N | K82N/N84S/R150Y/R170E/E240N | 2 | 512 | 1925 | 1967 | 539 | 18.1 | 396 | 0.54 |
| Y155F/K247N/N249S/R318Y/R338E/ | Y[155]F/K82N/N84S/R150Y/R170E/E240N/ | 2 | 617 | 1170 | 1221 | 685 | 8.1 | 745 | 0.83 |
| E410N | | | | | | | | | |
| R318Y/R338E/R403E/E410S | R150Y/R170E/R233E/E240S | 2 | 382 | 2897 | 2971 | 395 | 14.7 | 184 | 0.34 |
| R318Y/R338E/E410S | R150Y/R170E/E240S | 2 | 356 | 488 | 511 | 326 | 7.7 | 1066 | 2.08 |
| K228N/K247N/N249S | K63N/K82N/N84S | 2 | 662 | 3390 | 3578 | 753 | 19.6 | 268 | 0.28 |
| D104N/K106S/Y155F/K228N/K247N/ | D[104]N/K[106]S/Y[155]F/ | 3 | 781 ± 55.2 | 6110 ± 1900 | 6610 ± 1950 | 939 ± 48.2 | 18.5 ± 3.84 | 183 ± 63.3 | 0.160 ± 0.045 |
| N249S | K63N/K82N/N84S | | | | | | | | |
| D104N/K106S/K228N/K247N/N249S | D[104]N/K[106]S/K63N/K82N/N84S | 2 | 758 | 3792 | 4035 | 838 | 17.9 | 271 | 0.25 |
| Y155F/K228N/K247N/N249S | Y[155]F/K63N/K82N/N84S | 2 | 549 | 3002 | 3269 | 643 | 17.2 | 246 | 0.31 |
| K228N/K247N/N249S/R318Y/R338E/ | Y[155]F/I86S/R150Y/R170E/R233E/E240N/ | 3 | 599 ± 88.6 | 8570 ± 2830 | 9230 ± 2860 | 753 ± 120 | 21.7 ± 3.19 | 96.6 ± 15.4 | 0.115 ± 0.030 |
| R403E/E410N | | | | | | | | | |
| D104N/K106S/K228N/K247N/N249S/ | D[104]N/K[106]S/K63N/K82N/N84S/ | 3 | 806 ± 88.6 | 9330 ± 2830 | 9990 ± 2860 | 912 ± 120 | 24.4 ± 3.19 | 116 ± 15.4 | 0.100 ± 0.030 |
| R318Y/R338E/R403E/E410N | R150Y/R170E/R233E/E240N | | | | | | | | |
| Y155F/K228N/K247N/N249S/R318Y/ | Y[155]F/K63N/K82N/N84S/R150Y/R170E/ | 1 | 559 | 10704 | 11042 | 710 | 27.3 | 73 | 0.09 |
| R338E/R403E/E410N | R233E/E240N | | | | | | | | |
| R318Y/R338E/R403E/E410N/T412V | R150Y/R170E/R233E/E240N/T242V | 2 | 424 | 4730 | 4892 | 456 | 20.0 | 124 | 0.20 |
| R318Y/R338E/R403E/E410N/T412A | R150Y/R170E/R233E/E240N/T242A | 2 | 380 | 4994 | 5115 | 439 | 17.5 | 107 | 0.20 |
| R318Y/R338E/R403E/T412A | R150Y/R170E/R233E/T242A | 3 | 399 ± 88.1 | 4320 ± 2380 | 4500 ± 2360 | 477 ± 108 | 19.7 ± 0.684 | 144 ± 47.8 | 0.270 ± 0.145 |
| R318Y/R338E/T412A | R150Y/R170E/T242A | 2 | 462 | 1674 | 1691 | 401 | 13.6 | 398 | 0.60 |
| R318Y/R338E/E410N/T412V | 150Y/R170E/E240N/T242V | 2 | 251 | 524 | 555 | 226 | 16.3 | 772 | 2.31 |
| N260S/R318Y/R338E/R403E/E410N | N95S/R150Y/R170E/R233E/E240N | 2 | 583 | 6821 | 7488 | 743 | 23.9 | 111 | 0.13 |
| D104N/K106S/N260S/R318Y/R338E/ | D[104]N/K[106]S/N95S/R150Y/ | 2 | 779 | 7100 | 7728 | 999 | 17.2 | 145 | 0.12 |
| R403E/E410N | R170E/R233E/E240N | | | | | | | | |
| Y155F/N260S/R318Y/R338E/R403E/ | Y[155]F/N95S/R150Y/R170E/R233E/E240N | 2 | 628 | 5214 | 5465 | 758 | 21.4 | 167 | 0.21 |
| E410N | | | | | | | | | |
| R318Y/R338E/N346D/R403E/E410N | R150Y/R170E/N178D/R233E/E240N | 2 | 474 | 7623 | 8140 | 575 | 25.2 | 86 | 0.12 |
| Y155F/R318Y/R338E/N346D/R403E/ | Y[155]F/R150Y/R170E/N178D/R233E/E240N | 2 | 540 | 5039 | 5172 | 641 | 18.2 | 154 | 0.20 |
| E410N | | | | | | | | | |
| K247N/N249S/N260S | K82N/N84S/N95S | 2 | 549 | 4156 | 4262 | 632 | 17.4 | 186 | 0.23 |
| Y155F/K247N/N249S/N260S | Y[155]F/K82N/N84S/N95S | 2 | 691 | 3857 | 4085 | 814 | 24.0 | 244 | 0.22 |
| D104N/K106S/K247N/N249S/N260S | D[104]N/K[106]S/K82N/N84S/N95S | 2 | 712 | 4187 | 4458 | 859 | 16.5 | 235 | 0.23 |
| D104N/K106S/Y155F/K247N/N249S/ | D[104]N/K[106]S/Y[155]F/ | 2 | 680 | 7026 | 7423 | 856 | 23.3 | 134 | 0.14 |
| N260S | K82N N84S/N95S/ | | | | | | | | |
| K247N/N249S/N260S/R318Y/R338E/ | K82N/N84S/N95S/R150Y/R170E/R233E/E240N | 2 | 691 | 6353 | 6737 | 875 | 18.9 | 149 | 0.13 |
| R403E/E410N | | | | | | | | | |
| Y155F/K247N/N249S/N260S/R318Y/ | Y[155]F/K82N/N84S/N95S/R150Y/R170E/ | 1 | 1038 | 8401 | 9376 | 1068 | 21.0 | 160 | 0.11 |
| R338E/R403E/E410N | R233E/E240N | | | | | | | | |
| R318Y/R338E/T343R/R403E/E410N | T175R/R233E/E240N/R150Y/R170E | 2 | 531 | 3766 | 3862 | 560 | 20.5 | 200 | 0.27 |
| Y155F/R318Y/R338E/T343R/R403E/ | Y[155]F/R150Y/R170E/T175R/R233E/E240N | 1 | 182 | 3223 | 4335 | 259 | 20.5 | 61 | 0.23 |
| E410N | | | | | | | | | |
| D104N/K106S/R318Y/R338E/T343R/ | D[104]N/K[106]S/R150Y/R170E/ | 3 | 666 ± 89.9 | 7270 ± 729 | 7550 ± 708 | 699 ± 88.0 | 21.7 ± 4.71 | 128 ± 21.1 | 0.133 ± 0.013 |
| R403E/E410N | T175R/R233E/E240N | | | | | | | | |
| R338E/T343R | R170E/T175R | 2 | 534 | 798 | 813 | 453 | 12.8 | 949 | 1.23 |
| T343R/N346Y | T175R/N178Y | 3 | 276 ± 19.9 | 1080 ± 331 | 1100 ± 333 | 228 ± 7.76 | 12.3 ± 5.14 | 394 ± 156 | 0.989 ± 0.360 |
| R318Y/R338E/N346Y/R403E/E410N | R150Y/R170E/N178Y/R233E/E240N | 2 | 324 | 2394 | 2487 | 335 | 24.7 | 189 | 0.40 |
| R318Y/R338E/T343R/N346Y/R403E/ | R150Y/R170E/T175R/N178Y/R233E/E240N | 2 | 303 | 3569 | 3691 | 329 | 22.2 | 118 | 0.27 |
| E410N | | | | | | | | | |
| T343R/N346D | T175R/N178D | 2 | 388 | 2903 | 2917 | 356 | 17.0 | 192 | 0.34 |
| R318Y/R338E/T343R/N346D/R403E/ | R150Y/R170E/T175R/N178D/R233E/E240N | 2 | 450 | 6645 | 6717 | 506 | 20.7 | 97 | 0.15 |
| E410N | | | | | | | | | |
| R318Y/R338E/Y345A/R403E/E410N | R150Y/R170E/Y177A/R233E/E240N | 1 | 475 | 4989 | 5058 | 511 | 22.3 | 135 | 0.20 |
| R318Y/R338E/Y345A/N346D/R403E/ | R150Y/R170E/Y177A/N178D/R233E/E240N | 2 | 492 | 6249 | 6347 | 607 | 22.1 | 112 | 0.16 |
| E410N | | | | | | | | | |
| Y155F/K247N/N249S/R318Y/R338E/ | Y[155]F/K82N/N84S/R150Y/R170E/R233E | 2 | 622 | 10477 | 10973 | 791 | 26.9 | 85 | 0.10 |
| R403E | | | | | | | | | |
| K247N/N249S/R318Y/R338E/R403E | K82N/N84S/R150Y/R170E/R233E | 2 | 805 | 8099 | 8569 | 814 | 20.0 | 137 | 0.12 |
| Y155F/K247N/N249S/R338E/R403E/ | Y[155]F/K82N/N84S/R170E/R233E/E240N | 2 | 618 | 9233 | 9709 | 801 | 22.4 | 92 | 0.10 |
| E410N | | | | | | | | | |
| R318Y/R338E/T343R/R403E | R150Y/R170E/T175R/R233E | 2 | 421 | 6107 | 6153 | 473 | 19.9 | 99 | 0.16 |
| R318Y/R338E/T343R/E410N | R150Y/R170E/T175R/E240N | 2 | 529 | 793 | 815 | 391 | 5.6 | 931 | 1.23 |
| R150Y/T343R/R403E/E410N | R150Y/T175R/R233E/E240N | 2 | 431 | 5020 | 5060 | 434 | 20.7 | 130 | 0.21 |
| R170E/T343R/R403E/E410N | R170E/T175R/R233E/E240N | 2 | 484 | 5008 | 5060 | 450 | 19.8 | 141 | 0.20 |
| Y155F/R338E/T343R/R403E/E410N | Y[155]F/R170E/T175R/R233E/E240N | 2 | 628 | 5406 | 5509 | 521 | 17.9 | 164 | 0.18 |
| Y155F/K247N/N249S/R318Y/R338E/ | K82N/N84S/R150Y/R170E/T175R/R233E/E240N | 2 | 513 | 9067 | 9267 | 642 | 24.7 | 82 | 0.11 |
| T343R/R403E/E410N | | | | | | | | | |
| K247N/N249S/R318Y/R338E/T343R/ | K82N/N84S/R150Y/R170E/T175R/R233E/E240N | 2 | 536 | 8604 | 8824 | 672 | 24.4 | 89 | 0.12 |
| R403E/E410N | | | | | | | | | |
| Y155F/K228N/I251S/R318Y/R338E/ | Y[155]F/K63N/I86S/R150Y/R170E/ | 2 | 780 | 9033 | 9557 | 854 | 20.5 | 123 | 0.11 |
| R403E/E410N | R233E/E240N | | | | | | | | |
| N260S/R318Y/R338E/T343R/R403E/ | Y[155]F/N95S/R150Y/R170E/T175R/ | 2 | 539 | 8325 | 8537 | 675 | 24.0 | 92 | 0.12 |
| E410N | R233E/E240N | | | | | | | | |
| Y155F/N260S/R318Y/R338E/T343R/ | Y[155]F/N95S/R150Y/R170E/T175R/ | 1 | 578 | 3266 | 6295 | 733 | 20.4 | 133 | 0.16 |
| R403E/E410N | R233E/E240N | | | | | | | | |
| K228N/K247N/N249S/R318Y/R338E/ | K63N/K82N/N84S/R150Y/R170E/T175R/R233E/ | 2 | 753 | 8972 | 9391 | 757 | 26.0 | 117 | 0.11 |
| T343R/R403E/E410N | E240N | | | | | | | | |
| Y155F/R338E/T343R/R403E | Y[155]F/R170E/T175R/R233E | 2 | 503 | 5350 | 5412 | 506 | 16.7 | 135 | 0.19 |
| Y155F/R338E/T343R/R403E/E410S | Y[155]F/R170E/T175R/R233E/E240S | 2 | 589 | 5447 | 5546 | 526 | 22.9 | 156 | 0.18 |
| Y155F/N260S/R338E/T343R/R403E | Y[155]F/N95S/R170E/T175R/R233E | 2 | 485 | 9590 | 9749 | 619 | 24.0 | 74 | 0.10 |
| Y155F/I251S/R338E/T343R/R403E | Y[155]F/I86S/R170E/T175R/R233E | 2 | 732 | 7531 | 7926 | 807 | 21.0 | 134 | 0.13 |
| R318Y/R338E/T343R/R403E/E410S | R150Y/R170E/T175R/R233E/E240S | 2 | 618 | 4657 | 4728 | 466 | 19.9 | 199 | 0.23 |
| Y155F/K247N/N249S/T343R/R403E | Y[155]F/K82N/N84S/T175R/R233E | 2 | 866 | 7007 | 7391 | 751 | 18.3 | 169 | 0.14 |
| K247N/N249S/R338E/T343R/R403E/ | K82N/N84S/R170E/T175R/R233E/E240N | 2 | 804 | 9554 | 10051 | 776 | 20.4 | 116 | 0.10 |
| E410N | | | | | | | | | |
| Y155F/K247N/N249S/R318Y/R338E | Y[155]F/K82N/N84S/R150Y/R170E | 2 | 662 | 2965 | 3048 | 578 | 13.6 | 313 | 0.33 |
| Y155F/K247N/N249S/R338E/R403E | Y[155]F/K82N/N84S/R170E/R233E | 1 | 717 | 8404 | 8790 | 783 | 16.9 | 118 | 0.11 |
| Y155F/K247N/N249S/R338E/T343R/ | Y[155]F/K82N/N84S/R170E/T175R/ | 2 | 676 | 7455 | 7702 | 676 | 20.3 | 131 | 0.13 |
| R403E | R233E | | | | | | | | |
| K247N/N249S/T343R/R403E/E410N | K82N/N84S/T175R/R233E/E240N | 2 | 680 | 7758 | 8085 | 747 | 18.0 | 122 | 0.13 |

### Example 7

### In vivo assessment of FIX polypeptide procoagulant activity

Mouse models of hemophilia B, using mice deficient in FIX (FIX^{-/-} mice), were established to assess the procoagulant activity of FIX polypeptides. The mice were treated with FIX polypeptide and the amount of blood lost in 20 minutes was measured to determine the procoagulant activity of the FIX polypeptides.

### A. In Vivo Assessment of Wild-Type FIX Procoagulant Activity

Male FIX^{-/-} mice were anesthetized by intraperitoneal administration of a ketamine/xylazine cocktail (45 mg/ml and 3.6 mg/ml in saline) and placed on a heated platform (39° C.) to ensure there was no drop in body temperature. The procedure room was kept at a temperature of 82° F. Ten minutes prior to tail cut the tail was immersed in 10 mL of pre-warmed PBS (15 mL centrifuge tube; 39° C.). Seven to fifteen mice were injected with recombinant human FIX (Benefix® Coagulation Factor IX (Recombinant), Wyeth) or modified FIX polypeptides diluted in a buffer that was the same as that of Benefix® Coagulation Factor IX (Recombinant) (0.234% sodium chloride, 8 mM L-histidine, 0.8% sucrose, 208 mM glycine, 0.004% polysorbate 80) via the tail vein in a single injection. A negative control group of mice received buffer only. In instances where the injection was missed, the animal was excluded from the study.

Injection with FIX polypeptide or buffer was made 5 minutes prior to tail cut. The tail cut was made using a razor blade 5 mm from the end of the tail and blood was collected into PBS for a period of 20 minutes. At the end of the collection period, total blood loss was assessed. The collection tubes were mixed and a 1 ml aliquot of each sample was taken and assayed for hemoglobin content. Triton X-100 was diluted 1 in 4 in sterile water and 100 μL was added to the 1 mL samples to cause hemolysis. The absorbance of the samples was then measured at a wavelength of 546 nm. To calculate the amount of blood lost, the absorbance was read against a standard curve generated by measuring the absorbance at 546 nm of known volumes of murine blood, diluted in PBS and hemolyzed as above with Triton X 100. Values are expressed as Mean±SEM.

1. Dose Response Study Assessing Wild-Type FIX Coagulant Activity

Dose response studies to assess the coagulant activity of Benefix® Coagulation Factor IX (Recombinant) at 0.03, 0.1, 0.3 and 1 mg/kg in FIX^{-/-} mice were performed. In this experiment, the blood loss in the buffer-only group was 835.42±24.55 μl, which was significantly reduced by Benefix® Coagulation Factor IX (Recombinant) treatment at 0.1, 0.3 and 1 mg/kg (to 558.59±56.63 415.81±66.72 μL and 270.75±57.48 p<0.05 using Kruskal-Wallis followed by Dunn's post test). At the lowest dose tested of 0.03 mg/kg the value was 731.66±59.16 μL. Calculated ED₅₀ values using non-linear regression are shown in Table 37 below.

2. Dose Response Assessing the Coagulant Activity of FIXa-R318Y/R338E/R403E/E410N, FIXa-R318Y/R338E/E410N and FIXa-Y155F/K247N/N249S/R318Y/R338E/R403E/E410N

Dose response studies were conducted in which the coagulant activity of FIXa-R318Y/R338E/R403E/E410N (R150Y/R170E/R233E/E240N by chymotrypsin numbering), FIXa-R318Y/R338E/E410N (R150Y/R170E/E240N by chymotrypsin numbering) and FIXa-Y155F/K247N/N249S/R318Y/R338E/R403E/E410N (Y[155]F/K82N/N84S/R150Y/R170E/R233E/E240N by chymotrypsin numbering) at different doses were assessed.

Treatment with FIXa-R318Y/R338E/R403E/E410N resulted in significant inhibition of blood loss at 0.01, 0.03, 0.1, 0.3 and 1 mg/kg (434.65±73.75 497.28±50.92 230.81±39.67 261.94±58.79 μL and 251.56±41.81 respectively) compared to the buffer-only control (811.45±26.63 μL; p<0.05 using Kruskal-Wallis followed by Dunn's post test). Reducing the dose to 0.003 mg/kg led to blood loss values nearer control levels, of 786.83±44.39 μL.

Treatment with FIXa-R318Y/R338E/E410N also resulted in significant inhibition of blood loss at 0.03, 0.1, 0.3 and 1 mg/kg (571.67±50.45 425.42±43.65 263.47±42.66 μL and 78.19±13.42 respectively) compared to the buffer-only control (845.14±23.63 μL; p<0.05 using Kruskal-Wallis followed by Dunn's post test). Reducing the dose to 0.001 mg/kg led to blood loss values nearer control levels, of 777.16±53.72 μL.

Treatment with FIXa-Y155F/K247N/N249S/R318Y/R338E/R403E/E410N resulted in the most significant inhibition of blood loss of the mutants tested: 460.03±74.60 393.48±75.16 μL and 157.28±28.89 μL at 0.01, 0.03 and 0.1 mg/kg, respectively, compared to the buffer-only control (851.38±44.25 p<0.05 using Kruskal-Wallis followed by Dunn's post test). Calculated ED₅₀ values using non-linear regression are shown in Table 37 below.

TABLE 37

| Mutation | Mutation | | | Blood Loss; |
|---|---|---|---|---|
| (Mature FIX | (chymotrypsin | | n | ED50 |
| numbering) | numbering | n/group | (expts) | (mg/kg) |
| BeneFIX ® Coagulation | BeneFIX ® Coagulation | 7-20 | 2 | 0.2 |
| FIX (T148A) | FIX (T[148]A) | | | |
| R318Y/R338E/R403E/ | R150Y/R170E/R233E/ | 19-38 | 3 | 0.02 |
| E410N | E240N | | | |
| R318Y/R338E/E410N | R150Y/R170E/E240N | 8-42 | 4 | 0.06 |
| Y155F/K247N/N249S/ | Y[155]F/K82N/N84S/ | 18-21 | 2 | 0.01 |
| R318Y/R338E/R403E/ | R150Y/R170E/R233E/ | | | |
| E410N | E240N | | | |

3. Duration Response Assessing Wild-Type FIX Coagulant Activity

Studies were performed to assess the duration of effect of Benefix® Coagulation Factor IX (Recombinant) at 0.5 mg/kg in FIX^{-/-} mice. Mice were dosed intravenously at 48 hr, 24 hr, 16 hr, 8 hr, 4 hr, 2 hr, 30 min and 5 min prior to tail cut. In this experiment, inhibition from the control group was determined where the control group was set at 0% inhibition. Inhibition of blood loss was 59.7±11.9%, 48.25±12.84%, 57.74±9.10%, 56.04±8.46%, 32.09±7.92%, 12.94±7.33%, 38.75±11.47% and 0.64±11.3% at 5 min, 30 min, 2, 4, 8, 16, 24 and 48 hr, respectively from vehicle control (Mean and SEM, n=8-33 mice, from 3 experiments).

4. Duration Response Assessing FIXa-R318Y/R338E/R403E/E410N Coagulant Activity

Studies were performed to assess the duration of effect of FIXa-R318Y/R338E/R403E/E410N at 0.5 mg/kg in FIX^{-/-} mice. Mice were dosed i.v. at 96 hr, 72 hr, 48 hr, 32 hr, 24 hr, 16 hr, 8 hr, 4 hr, 2 hr, 30 min and 5 min prior to tail cut. In this experiment, inhibition from the control group was determined where the control group was set at 0% inhibition. Inhibition of blood loss was 93.26±2.04%, 96.30±3.70%, 85.86±6.52%, 69.4±9.92%, 89.05±3.69%, 78.48±8.71%, 63.33±6.70%, 47.97±10.07%, 3.1±8.22%, −13.52±10.59% and −12.82±7.31% at 5 min, 30 min, 2, 4, 8, 16, 24, 32, 48, 72 and 96 hr, respectively from vehicle control (Mean and SEM, n=8-45 mice, from 4 experiments).

Note on the FIX^{-/-} Mice:

The FIX knockout colony of mice was generated by in vitro fertilization using cryo-preserved sperm from male FIX knock out mice. All offspring were genotyped using PCR-based protocols to select those animals that contained a FIX knock-out allele. Further crossings of these animals and their offspring (after PCR-based genotyping) produced FIX knock-out animals (i.e., hemizygous males and homozygous females because the FIX gene is on the X chromosome), as confirmed by PCR. After PCR confirmation of the genotype of all members of this initial FIX colony, PCR confirmation of all colony offspring was ceased since legitimate knock-out animals can only produce knock-out offspring. “Retired breeders” from the colony were, however, genotyped on several occasions. Approximately 7 months after genotyping of all colony offspring was ceased, genotyping of retired breeders clearly indicated the presence of non-knock-out (or wild-type) animals in the colony. Based on this result, all members of the knock-out colony were genotyped and any non-knock-out animals were identified and eliminated from the colony. The results of the colony genotyping indicated that 19% of the male mice were wild type and 4% of the male animals were ambiguous due to poor DNA preparations. Both the wild type and “ambiguous” males (and females) were eliminated from the colony.

Thus, the FIX knockout colony was contaminated at some point with one or more non-knock-out animals and therefore contained a small fraction of non-knock out animals that increased over time until between 19-23% of the males in the colony contained a wild type FIX gene (in vivo experiments use male mice only). With respect to the FIX data generated and reported in this application, all of the in vitro data is unaffected. With respect to in vivo data, it is assumed and expected that the contamination affected all compounds similarly and therefore does not affect either the rank order of variants or their comparison to BeneFIX. Since the contaminating animals already had endogenous FIX, they would lose much less blood in the efficacy and duration experiments than true hemophilic animals and would benefit much less from administration of exogenous FIX, therefore increasing the “spread” or variability of data for all compounds. The contamination also could make all the compounds appear slightly less potent than they actually are, but their ratio to BeneFIX® should not be altered (i.e., the potency and duration advantage of our lead molecules should be unaffected).

### B. In Vivo Assessment of Wild-Type FIX Procoagulant Activity—New Colony Data

The data described below comes from a new colony, rebuilt from the confirmed FIX−/− mice described above. Mice were double confirmed by genotyping before being used as breeders. All data described below comes from mice born from breeding units where parents have been double confirmed. All replacement breeders are also double confirmed as FIX−/− prior to initiation of new breeding units.

Male FIX^{-/-} mice were anesthetized by intraperitoneal administration of a ketamine/xylazine cocktail (45 mg/ml and 3.6 mg/ml in saline) and placed on a heated platform (39° C.) to ensure there was no drop in body temperature. The procedure room was kept at a temperature of 82° F. Ten minutes prior to tail cut the tail was immersed in 10 mL of pre-warmed PBS (15 mL centrifuge tube; 39° C.). Seven to fifteen mice were injected with recombinant human FIX (Benefix® Coagulation Factor IX (Recombinant), Wyeth) or modified FIX polypeptides diluted in a buffer that was the same as that of Benefix® Coagulation Factor IX (Recombinant) (0.234% sodium chloride, 8 mM L-histidine, 0.8% sucrose, 208 mM glycine, 0.004% polysorbate 80) via the tail vein in a single injection. A negative control group of mice received buffer only. In instances where the injection was missed, the animal was excluded from the study.

Injection with FIX polypeptide or buffer was made 5 minutes prior to tail cut. The tail cut was made using a razor blade 5 mm from the end of the tail and blood was collected into PBS for a period of 20 minutes. At the end of the collection period, total blood loss was assessed. The collection tubes were mixed and a 1 ml aliquot of each sample was taken and assayed for hemoglobin content. Triton X-100 was diluted 1 in 4 in sterile water and 100 μL was added to the 1 mL samples to cause hemolysis. The absorbance of the samples was then measured at a wavelength of 546 nm. To calculate the amount of blood lost, the absorbance was read against a standard curve generated by measuring the absorbance at 546 nm of known volumes of murine blood, diluted in PBS and hemolyzed as above with Triton X 100. Values are expressed as Mean±SEM.

1. Dose Response Studies Assessing FIX Coagulant Activity

Dose response studies to assess the coagulant activity of Benefix® Coagulation Factor IX (Recombinant) and FIX polypeptides at varying doses in FIX^{-/-} mice were performed. In these experiments ED₅₀ values were calculated using non-linear regression and are shown in Table 38 below.

TABLE 38
Dose Response ED₅₀ values

| | Mutation | | | Average |
|---|---|---|---|---|
| | (Chymotrypsin | n/group/ | N | ED50 |
| Mutation | numbering) | expt | (expts) | (mg/kg) |
| BeneFIX ® FIX | BeneFIX ® FIX | 10-14 | 2 | 0.4 |
| WT | Catalyst Biosciences WT | 8-15 | 4 | 1.6 |
| T148A | T[148]A | 10-15 | 2 | 1.0 |
| R318Y/R338E/E410N | R150Y/R170E/E240N | 10-13 | 2 | 0.14 |
| R318Y/R403E/E410N | R150Y/R233E/E240N | 13-15 | 2 | 0.095 |
| R318Y/R338E/R403E/E410N | R150Y/R170E/R233E/E240N | 7-14 | 6 | 0.02 |
| D104N/K106S/Y155F/R318Y/ | D[104]N/K[106]S/Y[155] | 9-14 | 4 | 0.05 |
| R338E/R403E/E410N | F/R150Y/R170E/R233E/E240N | | | |
| T343R | T175R | 9-15 | 4 | 0.9 |
| Y155F/K228N/R318Y/R338E/ | Y[155]F/K63N/R150Y/R170E/ | 10-14 | 2 | 0.08 |
| R403E/E410N | R233E/E240N | | | |
| I251S/R318Y/R338E/E410N | I86S/R150Y/R170E/E240N | 9-18 | 3 | 1.0 |
| K247N/N249S/R318Y/R338E/ | K82N/N84S/R150Y/R170E/ | 9-14 | 4 | 0.06 |
| R403E/E410N | R233E/E240N | | | |
| Y155F/K247N/N249S/R318Y/ | Y[155]F/K82N/N84S/R150Y/ | 9-15 | 4 | 0.03 |
| R338E/R403E/E410N | R170E/R233E/E240N | | | |
| A103N/N105S/K247N/N249S/ | A[103]N/N[105]S/K82N/N84S/ | 8-10 | 2 | 0.08 |
| R318Y/R338E/R403E/E410N | R150Y/R170E/R233E/E240N | | | |
| D104N/K106S/Y155F/K247N/ | D[104]N/K[106]S/Y[155] | 12-15 | 2 | 0.055 |
| N249S/R318Y/R338E/R403E/ | F/K82N/N84S/R150Y/R170E/ | | | |
| E410N | R233E/E240N | | | |
| R318Y/R338E/R403E/E410S | R150Y/R170E/R233E/E240S | 10-15 | 2 | 0.055 |
| D104N/K106S/Y155F/K228N/ | D[104]N/K[106]S/Y[155] | 10-12 | 1 | 1.64 |
| K247N/N249S | F/K63N/K82N/N84S | | | |
| K228N/K247N/N249S/R318Y/ | K63N/K82N/N84S/R150Y/ | 8-15 | 5 | 0.08 |
| R338E/R403E/E410N | R170E/R233E/E240N | | | |
| D104N/K106S/K228N/K247N/ | D[104]N/K[106]S/K63N/ | 13-15 | 2 | 0.125 |
| N249S/R318Y/R338E/R403E/ | K82N/N84S/R150Y/R170E/ | | | |
| E410N | R233E/E240N | | | |
| Y155F/K228N/K247N/N249S/ | Y[155]F/K63N/K82N/N84S/ | 12-15 | 2 | 0.035 |
| R318Y/R338E/R403E/E410N | R150Y/R170E/R233E/E240N | | | |
| R318Y/R338E/R403E/E410N/ | R150Y/R170E/R233E/E240N/ | 8-14 | 3 | 0.03 |
| T412V | T242V | | | |
| R318Y/R338E/R403E/E410N/ | R150Y/R170E/R233E/E240N/ | 11-15 | 2 | 0.04 |
| T412A | T242A | | | |
| K247N/N249S/N260S/R318Y/ | K82N/N84S/N95S/R150Y/ | 8-15 | 4 | 0.26 |
| R338E/R403E/E410N | R170E/R233E/E240N | | | |
| Y155F/ | Y[155]F/K82N/N84S/N95S/ | 13-15 | 3 | 0.06 |
| K247N/N249S/N260S/R318Y/ | R150Y/R170E/R233E/E240N | | | |
| R338E/R403E/E410N | | | | |
| R318Y/R338E/T343R/R403E/ | R150Y/R170E/T175R/R233E/ | 7-15 | 5 | 0.025 |
| E410N | E240N | | | |
| Y155F/R318Y/R338E/T343R/ | Y[155]F/R150Y/R170E/ | 10-14 | 2 | 0.0045 |
| R403E/E410N | T175R/R233E/E240N | | | |
| D104N/K106S/R318Y/R338E/ | D[104]N/K[106]S/R150Y/ | 10-15 | 3 | 0.07 |
| T343R/R403E/E410N | R170E/T175R/R233E/E240N | | | |
| R338E/T343R | R170E/T175R | 11-14 | 2 | 0.83 |
| R318Y/R338E/T343R/N346Y/ | R150Y/R170E/T175R/N178Y/ | 9-13 | 3 | 0.03 |
| R403E/E410N | R233E/E240N | | | |
| Y155F/K247N/N249S/R318Y/ | Y[155]F/K82N/N84S/R150Y/ | 11-15 | 2 | 0.145 |
| R338E/R403E | R170E/R233E | | | |
| Y155F/K247N/N249S/R338E/ | Y[155]F/K82N/N84S/R170E/ | 12-15 | 3 | 0.08 |
| R403E/E410N | R233E/E240N | | | |
| R318Y/R338E/T343R/R403E | R150Y/R170E/T175R/R233E | 10-15 | 2 | 0.025 |
| Y155F/R318Y/R338E/T343R/ | Y[155]F/R150Y/R170E/ | 10-14 | 2 | 0.007 |
| R403E | T175R/R233E | | | |
| R318Y/R338E/T343R/E410N | R150Y/R170E/T175R/E240N | 11-15 | 5 | 0.13 |
| R318Y/T343R/R403E/E410N | R150Y/T175R/R233E/E240N | 10-15 | 2 | 0.03 |
| Y155F/R318Y/T343R/R403E/ | Y[155]F/R150Y/T175R/ | 13-15 | 2 | 0.07 |
| E410N | R233E/E240N | | | |
| R338E/T343R/R403E/E410N | R170E/T175R/R233E/E240N | 11-15 | 2 | 0.045 |
| Y155F/R338E/T343R/R403E/ | Y[155]F/R170E/T175R/ | 10-15 | 2 | 0.055 |
| E410N | R233E/E240N | | | |
| Y155F/K247N/N249S/R318Y/ | Y[155]F/K82N/N84S/R150Y/ | 11-15 | 2 | 0.04 |
| R338E/T343R/R403E/E410N | R170E/T175R/R233E/E240N | | | |
| K247N/N249S/R318Y/R338E/ | K82N/N84S/R150Y/R170E/ | 11-15 | 2 | 0.035 |
| T343R/R403E/E410N | T175R/R233E/E240N | | | |
| K228N/I251S/R318Y/R338E/ | K63N/I86S/R150Y/R170E/ | 10-15 | 3 | 0.01 |
| R403E/E410N | R233E/E240N | | | |
| Y155F/K228N/I251S/R318Y/ | Y[155]F/K63N/I86S/R150Y/ | 13-15 | 2 | 0.04 |
| R338E/R403E/E410N | R170E/R233E/E240N | | | |
| N260S/R318Y/R338E/T343R/ | N95S/R150Y/R170E/T175R/ | 12-15 | 2 | 0.03 |
| R403E/E410N | R233E/E240N | | | |
| Y155F/N260S/R318Y/R338E/ | Y[155]F/N95S/R150Y/R170E/ | 10-15 | 2 | 0.02 |
| T343R/R403E/E410N | T175R/R233E/E240N | | | |
| K228N/K247N/N249S/R318Y/ | K63N/K82N/N84S/R150Y/ | 12-15 | 3 | 0.03 |
| R338E/T343R/R403E/E410N | R170E/T175R/R233E/E240N | | | |
| Y155F/R338E/T343R/R403E | Y[155]F/R170E/T175R/R233E | 12-15 | 1 | 0.06 |
| R338E/T343R/R403E | R170E/T175R/R233E | 10-15 | 2 | 0.195 |
| Y155F/R338E/T343R/R403E/ | Y[155]F/R170E/T175R/ | 12-15 | 3 | 0.06 |
| E410S | R233E/E240S | | | |
| Y155F/N260S/R338E/T343R/ | Y[155]F/N95S/R170E/T175R/ | 12-15 | 1 | 0.1 |
| R403E | R233E | | | |
| Y155F/I251S/R338E/T343R/ | Y[155]F/I86S/R170E/T175R/ | 13-15 | 2 | 0.145 |
| R403E | R233E | | | |
| R318Y/R338E/T343R/R403E/ | R150Y/R170E/T175R/R233E/ | 11-15 | 2 | 0.015 |
| E410S | E240S | | | |
| Y155F/K247N/N249S/T343R/ | Y[155]F/K82N/N84S/T175R/ | 12-14 | 2 | 0.26 |
| R403E | R233E | | | |
| Y155F/K247N/N249S/R318Y/ | Y[155]F/K82N/N84S/R150Y/ | 10-14 | 2 | 0.006 |
| R338E/T343R/R403E | R170E/T175R/R233E | | | |
| K247N/N249S/R318Y/R338E/ | K82N/N84S/R150Y/R170E/ | 10-13 | 2 | 0.009 |
| T343R/R403E | T175R/R233E | | | |
| Y155F/K247N/N249S/R338E/ | Y[155]F/K82N/N84S/R170E/ | 12-13 | 1 | 0.2 |
| T343R/R403E/E410N | T175R/R233E/E240N | | | |
| K247N/N249S/R338E/T343R/ | K82N/N84S/R170E/T175R/ | 11-14 | 1 | 0.01 |
| R403E/E410N | R233E/E240N | | | |
| Y155F/K247N/N249S/R318Y/ | Y[155]F/K82N/N84S/R150Y/ | 13-15 | 2 | 0.04 |
| R338E | R170E | | | |
| K247N/N249S/R338E/T343R/ | K82N/N84S/R170E/T175R/ | 10-15 | 2 | 0.18 |
| R403E/E410N | R233E/E240N | | | |
| Y155F/K247N/N249S/R338E/ | Y[155]F/K82N/N84S/R170E/ | 12-15 | 2 | 0.22 |
| R403E | R233E | | | |
| Y155F/K247N/N249S/R318Y/ | Y[155]F/K82N/N84S/R150Y/ | 12-15 | 2 | 0.12 |
| R338E/T343R/E410N | R170E/T175R/E240N | | | |
| K247N/N249S/R318Y/R338E/ | K82N/N84S/R150Y/R170E/ | 11-14 | 2 | 0.12 |
| T343R/E410N | T175R/E240N | | | |
| Y155F/K247N/N249S/R318Y/ | Y[155]F/K82N/N84S/R150Y/ | 10-15 | 2 | 0.07 |
| T343R/R403E/E410N | T175R/R233E/E240N | | | |
| K247N/N249S/R318Y/T343R/ | K82N/N84S/R150Y/T175R/ | 14-15 | 1 | 0.02 |
| R403E/E410N | R233E/E240N | | | |
| Y155F/K247N/N249S/R318Y/ | Y[155]F/K82N/N84S/R150Y/ | 11-14 | 2 | 0.065 |
| T343R/R403E | T175R/R233E | | | |
| Y155F/K247N/N249S/R318Y/ | Y[155]F/K82N/N84S/R150Y/ | 12-15 | 1 | 0.25 |
| T343R/E410N | T175R/E240N | | | |
| Y155F/K247N/N249S/R338E/ | Y[155]F/K82N/N84S/R170E/ | 10-15 | 2 | 0.125 |
| T343R/R403E | T175R/R233E | | | |
| Y155F/K247N/N249S/T343R/ | Y[155]F/K82N/N84S/T175R/ | 13-14 | 1 | 0.1 |
| R403E/E410N | R233E/E240N | | | |
| Y155F/K247N/N249S/R318Y/ | Y[155]F/K82N/N84S/R150Y/ | 13-14 | 2 | 0.07 |
| R338E/T343R | R170E/T175R | | | |
| Y155F/K247N/N249S/T343R/ | Y[155]F/K82N/N84S/T175R/ | 11-15 | 1 | 0.11 |
| E410N | E240N | | | |

2. Duration Response Assessing Wild-Type FIX Coagulant Activity

Studies were performed to assess the duration of effect of Benefix® Coagulation Factor IX (Recombinant) at 0.5 mg/kg in FIX^{-/-} mice. Mice were dosed intravenously at 48 hr, 32 hr, 24 hr, 16 hr, 8 hr, 4 hr, 2 hr and 5 min prior to tail cut. In this experiment, inhibition from the control group was determined where the control group was set at 0% inhibition. Inhibition of blood loss was 68.6±5.8%, 64±6.98%, 54.7±6.13%, 43.4±6.86%, 13.7±5.53%, 24.9±6.11%, 11.7±4.88% and 5.6±4.17% at 5 min, 2, 4, 8, 16, 24, 32 and 48 hr, respectively from vehicle control (Mean and SEM, n=10-35 mice, from 3 experiments).

3. Duration Response Assessing FIX Polypeptide Procoagulant Activity

Studies were performed to assess the duration of effect of FIX-polypeptides at 0.5 mg/kg in FIX^{-/-} mice. Mice were dosed i.v. at 72 hr, 48 hr, 32 hr, 24 hr, 8 hr and 5 min prior to tail cut, or at 72 hr, 48 hr and 1 hr prior to tail cut. In these experiments, inhibition from the control group was determined where the control group was set at 0% inhibition. Inhibition of blood loss is shown as % inhibition (Mean and SEM) in Table 39.

TABLE 39
Inhibition of blood loss

| Mutation | | | Inhibition (% of vehicle (0) +/− SEM) at each time point (hrs) | | | | | | |
|---|---|---|---|---|---|---|---|---|---|
| (chymotrypsin numbering) | n/group | N (expt) | 0.08 | 1 | 8 | 24 | 32 | 48 | 72 |
| R150Y/R170E/R233E | 24-30 | 2 | 85 +/− 3.2 | | 88.8 +/− 2.8 | 59.5 +/− 7.3 | 71.8 +/− 7.0 | 40.2 +/− 7.8 | 7.8 +/− 5.2 |
| R150Y/R170E/E240N | 37-44 | 3 | 71.6 +/− 3.9 | | 85.0 +/− 3.8 | 59.4 +/− 6.8 | 55.3 +/− 6.1 | 21.0 +/− 6.2 | 27.7 +/− 7.3 |
| Y[155]F/R150Y/R170E/E240N | 26-29 | 2 | | 74.2 +/− 6.5 | | | | 56.8 +/− 9.0 | 15.6 +/− 8.2 |
| R150Y/R233E/E240N | 23-29 | 2 | 71.0 +/− 3.7 | | 71.4 +/− 6.6 | 31.1 +/− 6.1 | 15.8 +/− 4.3 | 4.8 +/− 5.4 | −0.4 +/− 2.9 |
| R150Y/R170E/R233E/E240N | 75-86 | 7 | 75.9 +/− 2 | | 82.7 +/− 2.6 | 58 +/− 4.8 | 63.6 +/− 4.4 | 31.1 +/− 4.9 | 3.5 +/− 2.7 |
| Y[155]F/R150Y/R170E/R233E/E240N | 25-30 | 2 | | 88.5 +/− 1.7 | | | | 22.2 +/− 8.2 | −17.6 +/− 3.6 |
| D[104]N/K[106]S/Y[155]F/R150Y/R170E/R233E/E240N | 35-44 | 3 | 70.8 +/− 3.0 | | 85.5 +/− 3.5 | 55.1 +/− 5.4 | 48.3 +/− 7.2 | 27.3 +/− 5.7 | 12.1 +/− 3.0 |
| T175R | 23-28 | 2 | 43.7 +/− 6.3 | | 30.9 +/− 6.6 | 23.8 +/− 3.8 | 12.3 +/− 6.1 | 14.8 +/− 7.1 | 3.4 +/− 3.1 |
| Y[155]F/K63N/R150Y/R170E/R233E/E240N | 36-43 | 3 | 65.2 +/− 3.0 | | 72.2 +/− 4.5 | 59.2 +/− 6.5 | 42.4 +/− 8.3 | 41.2 +/− 7.6 | 4.7 +/− 5.6 |
| K82N/N84S/R150Y/R170E/R233E/E240N | 37-41 | 3 | 78.7 +/− 2.5 | | 85.9 +/− 2.6 | 52.5 +/− 5.5 | 49.9 +/− 6.8 | 31.4 +/− 5.9 | 5.0 +/− 4.2 |
| Y[155]F/K82N/N84S/R150Y/R170E/R233E/E240N | 57-65 | 5 | 79.1 +/− 2.2 | | 79.5 +/− 2.7 | 66.7 +/− 4.0 | 61.1 +/− 4.8 | 38.2 +/− 5.2 | 17.1 +/− 4.0 |
| D[104]N/K[106]S/Y[155]F/K82N/N84S/R150Y/R170E/R233E/E240N | 20-29 | 2 | 71.2 +/− 4.5 | | 74.2 +/− 6.6 | 61.2 +/− 7.2 | 48.7 +/− 8.2 | 54.1 +/− 7.7 | 12.3 +/− 6.5 |
| K82N/N84S/R150Y/R170E/E240N | 23-28 | 2 | | 76.0 +/− 6.6 | | | | 26.2 +/− 8.7 | 22.3 +/− 7.1 |
| Y[155]F/K82N/N84S/R150Y/R170E/E240N | 26-30 | 2 | | 77.7 +/− 5.1 | | | | 16.0 +/− 7.3 | −2.2 +/− 4.3 |
| R150Y/R170E/R233E/E240S | 35-42 | 3 | 79.3 +/− 1.9 | | 75.6 +/− 4.6 | 51.0 +/− 5.4 | 48.3 +/− 6.5 | 12.3 +/− 5.3 | −5.6 +/− 2.4 |
| K63N/K82N/N84S/R150Y/R170E/R233E/E240N | 32-38 | 3 | 72.6 +/− 2.9 | | 78.6 +/− 3.7 | 44.2 +/− 7 | 53.9 +/− 7.1 | 42.9 +/− 6.9 | 10.4 +/− 5.4 |
| D[104]N/K[106]S/K63N/K82N/N84S/R150Y/R170E/R233E/E240N | 26-28 | 2 | 81.6 +/− 3.5 | | 86.0 +/− 3.6 | 46.8 +/− 8.0 | 59.7 +/− 7.7 | 33.8 +/− 8.3 | 26.2 +/− 5.8 |
| Y[155]F/K63N/K82N/N84S/R150Y/R170E/R233E/E240N | 23-29 | 2 | 85.5 +/− 2.2 | | 75.6 +/− 4.0 | 70.6 +/− 6.5 | 58.4 +/− 6.3 | 27.0 +/− 7.7 | 14.1 +/− 7.8 |
| R150Y/R170E/R233E/E240N/T242V | 40-44 | 3 | 69.5 +/− 3.2 | | 85.5 +/− 2.6 | 37.5 +/− 5.1 | 42.8 +/− 6.2 | 9.0 +/− 6.6 | −3.8 +/− 3.4 |
| R150Y/R170E/R233E/E240N/T242A | 29-38 | 3 | 81.3 +/− 2.5 | | 85.6 +/− 3.3 | 45.2 +/− 6.2 | 35.6 +/− 6.3 | 29.3 +/− 6.0 | 3.7 +/− 3.1 |
| K82N/N84S/N95S/R150Y/R170E/R233E/E240N | 20-28 | 2 | 46.4 +/− 6.6 | | 37.7 +/− 7.5 | 4.0 +/− 2.6 | 16.0 +/− 4.7 | 0.08 +/− 3.8 | −6.1 +/− 2.4 |
| Y[155]F/K82N/N84S/N95S/R150Y/R170E/R233E/E240N | 37-43 | 3 | 72.2 +/− 4.4 | | 69.1 +/− 5.4 | 47.0 +/− 6.1 | 44.3 +/− 6.2 | 27.0 +/− 6.4 | 8.1 +/− 5.3 |
| R150Y/R170E/T175R/R233E/E240N | 32-38 | 3 | 80.3 +/− 2.6 | | 78.2 +/− 3.8 | 68.3 +/− 5.5 | 69.4 +/− 6.0 | 23.2 +/− 7.2 | 4.9 +/− 5.8 |
| Y[155]F/R150Y/R170E/T175R/R233E/E240N | 21-27 | 2 | 84.8 +/− 2.5 | | 87.8 +/− 2.8 | 76.6 +/− 4.2 | 66.7 +/− 6.6 | 56.8 +/− 8.0 | 8.2 +/− 8.0 |
| D[104]N/K[106]S/R150Y/R170E/T175R/R233E/E240N | 26-30 | 2 | 80.4 +/− 2.8 | | 81.5 +/− 4.8 | 69.5 +/− 7.6 | 60.4 +/− 7.9 | 54.8 +/− 6.7 | 12.8 +/− 6.3 |
| R150Y/R170E/T175R/N178Y/R233E/E240N | 35-43 | 3 | 76.6 +/− 3.1 | | 85.1 +/− 3.3 | 43.9 +/− 5.7 | 47.9 +/− 6.8 | 14.9 +/− 6.2 | −12.1 +/− 2.9 |
| Y[155]F/K82N/N84S/R150Y/R170E/R233E | 24-30 | 2 | 76.2 +/− 3.0 | | 85.6 +/− 4.7 | 49.6 +/− 6.5 | 61.1 +/− 7.4 | 46.0 +/− 6.9 | 0.4 +/− 4.9 |
| K82N/N84S/R150Y/R170E/R233E | 27-29 | 2 | | 70.0 +/− 5.8 | | | | 18.8 +/− 6.3 | 2.1 +/− 2.7 |
| Y[155]F/K82N/N84S/R170E/R233E/E240N | 38-44 | 3 | 69.8 +/− 4.7 | | 78.4 +/− 4.1 | 56.4 +/− 5.9 | 58.4 +/− 5.8 | 51.1 +/− 6.6 | 26.9 +/− 5.4 |
| K82N/N84S/R170E/R233E/E240N | 28-30 | 2 | | 63.9 +/− 7.2 | | | | 16.7 +/− 6.3 | −7.0 +/− 2.0 |
| R150Y/R170E/T175R/R233E | 37-43 | 3 | 80.0 +/− 2.1 | | 83.5 +/− 3.5 | 62.1 +/− 5.6 | 62.6 +/− 5.3 | 50.5 +/− 5.9 | 1.9 +/− 4.0 |
| Y[155]F/R150Y/R170E/T175R/R233E | 24-28 | 2 | 80.4 +/− 3.0 | | 90.7 +/− 2.1 | 65.7 +/− 6.6 | 67.2 +/− 7.3 | 52.2 +/− 8.2 | 41.1 +/− 8.3 |
| R150Y/R170E/T175R/E240N | 35-44 | 3 | 65.5 +/− 4.7 | | 74.1 +/− 5.3 | 55.8 +/− 5.6 | 53.1 +/− 6.8 | 46.4 +/− 6.7 | 34.9 +/− 6.0 |
| R150Y/T175R/R233E/E240N | 29-30 | 2 | 74.1 +/− 3.6 | | 77.7 +/− 3.9 | 55.3 +/− 7.5 | 39.4 +/− 8.1 | 24.5 +/− 7.6 | 6.8 +/− 4.8 |
| Y[155]F/R150Y/T175R/R233E/E240N | 25-29 | 2 | | 92.7 +/− 2.1 | | | | 29.3 +/− 6.1 | 7.7 +/− 3.2 |
| R170E/T175R/R233E/E240N | 26-30 | 2 | 67 +/− 5.3 | | 87.4 +/− 4.2 | 55.9 +/− 8.7 | 47.2 +/− 8.6 | 33.0 +/− 8.4 | 9.2 +/− 5.3 |
| Y[155]F/R170E/T175R/R233E/E240N | 34-43 | 3 | 77.8 +/− 4.2 | | 90.8 +/− 2.8 | 68.6 +/− 5.2 | 61.3 +/− 5.8 | 35.6 +/− 8.3 | 5.9 +/− 5.0 |
| Y[155]F/K82N/N84S/R150Y/R170E/T175R/R233E/E240N | 39-43 | 3 | 76.0 +/− 3.0 | | 80.4 +/− 3.3 | 72.7 +/− 3.8 | 64.2 +/− 5.4 | 51.4 +/− 5.7 | 33.1 +/− 7.3 |
| K82N/N84S/R150Y/R170E/T175R/R233E/E240N | 42-44 | 3 | 83.0 +/− 2.4 | | 81.0 +/− 2.4 | 73.8 +/− 5.2 | 57.1 +/− 5.7 | 48.5 +/− 6.1 | 16.9 +/− 6.8 |
| K63N/I86S/R150Y/R170E/R233E/E240N | 21-26 | 2 | 71.9 +/− 3.6 | | 85.8 +/− 4.0 | 71.3 +/− 6.8 | 54.8 +/− 7.3 | 40.3 +/− 10.3 | 23.1 +/− 10.4 |
| Y[155]F/K63N/I86S/R150Y/R170E/R233E/E240N | 26-29 | 2 | 82.1 +/− 2.7 | | 83.6 +/− 3.7 | 65.6 +/− 5.5 | 57.2 +/− 7.9 | 38.4 +/− 8.9 | 16.5 +/− 7.7 |
| N95S/R150Y/R170E/T175R/R233E/E240N | 24-29 | 2 | 75.5 +/− 4.5 | | 76.6 +/− 4.3 | 82.2 +/− 5.8 | 84.7 +/− 3.9 | 41.6 +/− 8.6 | 20.1 +/− 6.0 |
| Y[155]F/N95S/R150Y/R170E/T175R/R233E/E240N | 21-27 | 2 | 85.2 +/− 2.5 | | 89.7 +/− 3.6 | 46.5 +/− 7.0 | 63.3 +/− 8.0 | 41.6 +/− 8.8 | 9.1 +/− 6.5 |
| K63N/K82N/N84S/R150Y/R170E/T175R/R233E/E240N | 34-45 | 3 | 83.9 +/− 1.8 | | 79.8 +/− 3.6 | 75.2 +/− 4.9 | 80.9 +/− 3.0 | 73.0 +/− 4.4 | 43.8 +/− 6.6 |
| Y[155]F/K63N/K82N/N84S/R150Y/R170E/T175R/R233E/E240N | 24-26 | 2 | | 84.6 +/− 3.4 | | | | 70.6 +/− 7.5 | 50.9 +/− 8.6 |
| Y[155]F/R170E/T175R/R233E | 22-30 | 2 | 81.9 +/− 3.6 | | 79.2 +/− 6.2 | 55.0 +/− 8.0 | 44.4 +/− 9.9 | 26.8 +/− 6.8 | −6.5 +/− 2.7 |
| R170E/T175R/R233E | 23-28 | 2 | 60.6 +/− 6.4 | | 86.5 +/− 4.3 | 35.6 +/− 8.3 | 35.8 +/− 8.5 | 18.9 +/− 6.8 | 12.1 +/− 6.0 |
| Y[155]F/R170E/T175R/R233E/E240S | 24-27 | 2 | 71.2 +/− 4.5 | | 77.8 +/− 5.3 | 54.6 +/− 8.2 | 58.3 +/− 8.1 | 21.9 +/− 7.1 | −11.0 +/− 3.4 |
| Y[155]F/N95S/R170E/T175R/R233E | 25-29 | 2 | 58.2 +/− 7.9 | | 65.5 +/− 8.3 | 48.2 +/− 10.0 | 29.3 +/− 9.3 | 21.0 +/− 6.7 | −14.8 +/− 5.3 |
| Y[155]F/I86S/R170E/T175R/R233E | 23-30 | 2 | 84.1 +/− 5.1 | | 90.9 +/− 2.7 | 76.6 +/− 6.4 | 62.4 +/− 6.7 | 55.2 +/− 7.9 | 23.7 +/− 6.5 |
| R150Y/R170E/T175R/R233E/E240S | 27-43 | 3 | 80.2 +/− 2.5 | | 87.1 +/− 3.2 | 76.9 +/− 4.0 | 67.9 +/− 5.6 | 48.3 +/− 5.5 | 21.0 +/− 5.0 |
| Y[155]F/K82N/N84S/T175R/R233E | 12-29 | 2 | 70.5 +/− 6.9 | 84.2 +/− 5.4 | 53.2 +/− 12.3 | 39.5 +/− 11.1 | 18.0 +/− 7.3 | 17.0 +/− 5.4 | −7.4 +/− 3.1 |
| Y[155]F/K82N/N84S/R150Y/R170E/T175R/R233E | 36-41 | 3 | 79.6 +/− 3.2 | | 90.5 +/− 2.4 | 73.8 +/− 4.6 | 75.0 +/− 5.0 | 74.4 +/− 4.7 | 27.5 +/− 6.5 |
| K82N/N84S/R150Y/R170E/T175R/R233E | 22-28 | 2 | 84.3 +/− 3.1 | | 91.8 +/− 1.4 | 60.1 +/− 6.7 | 54.0 +/− 8.1 | 43.6 +/− 8.8 | 35.7 +/− 8.7 |
| Y[155]F/K82N/N84S/R170E/T175R/R233E/E240N | 25-30 | 2 | | 91.1 +/− 1.8 | | | | 22.7 +/− 6.6 | 12.8 +/− 6.2 |
| K82N/N84S/R170E/T175R/R233E/E240N | 25-28 | 2 | | 82.7 +/− 4.5 | | | | 67.1 +/− 7.7 | 21.6 +/− 8.0 |
| Y[155]F/K82N/N84S/R150Y/R170E | 20-29 | 2 | | 83.3 +/− 3.9 | | | | 47.8 +/− 7.0 | 19.4 +/− 6.2 |
| Y[155]F/K82N/N84S/R150Y/T175R | 24-28 | 2 | | 43.6 +/− 6.5 | | | | 4.9 +/− 4.6 | 7.2 +/− 1.9 |
| Y[155]F/K82N/N84S/R170E/R233E | 15-30 | 2 | 47.2 +/− 8.0 | 64.7 +/− 9.7 | 90.8 +/− 4.5 | 78.4 +/− 7.5 | 49.2 +/− 11.5 | 19.7 +/− 7.9 | −5.8 +/− 4.2 |
| Y[155]F/K82N/N84S/R170E/T175R | 25-27 | 2 | | 70.5 +/− 7.0 | | | | 34.0 +/− 7.4 | 27.9 +/− 6.4 |
| Y[155]F/K82N/N84S/R150Y/R170E/T175R/E240N | 28-30 | 2 | | 73.7 +/− 6.7 | | | | 30.1 +/− 8.4 | 43.1 +/− 7.9 |
| K82N/N84S/R150Y/R170E/T175R/E240N | 25-29 | 2 | | 77.2 +/− 6.0 | | | | 29.5 +/− 7.2 | 29.0 +/− 5.5 |
| Y[155]F/K82N/N84S/R150Y/T175R/R233E/E240N | 26-28 | 2 | | 87.6 +/− 2.4 | | | | 42.6 +/− 8.6 | 14.5 +/− 6.4 |
| K82N/N84S/R150Y/T175R/R233E/E240N | 28-30 | 2 | | 91.3 +/− 2.6 | | | | 52.4 +/− 7.7 | 6.6 +/− 4.5 |
| Y[155]F/K82N/N84S/R170E/E240N | 25-30 | 2 | | 74.6 +/− 6.4 | | | | 30.1 +/− 7.1 | 12.4 +/− 6.3 |
| Y[155]F/K82N/N84S/R150Y/T175R/R233E | 27-30 | 2 | | 85.2 +/− 4.4 | | | | 31.1 +/− 7.8 | −7.9 +/− 2.6 |
| K82N/N84S/R150Y/T175R/E240N | 25-30 | 2 | | 51.9 +/− 8.2 | | | | 9.4 +/− 4.9 | 3.2 +/− 4.5 |
| Y[155]F/K82N/N84S/R170E/T175R/R233E | 27-29 | 2 | | 84.6 +/− 5.0 | | | | 26.8 +/− 8.5 | 10.9 +/− 6.9 |
| K82N/N84S/R170E/T175R/R233E | 27-29 | 2 | | 73.0 +/− 6.6 | | | | 27.3 +/− 7.7 | 23.4 +/− 5.6 |
| K82N/N84S/R170E/T175R/E240N | 24-29 | 2 | | 59.1 +/− 8.0 | | | | 29.6 +/− 7.4 | 12.2 +/− 5.2 |
| Y[155]F/K82N/N84S/T175R/R233E/E240N | 28-30 | 2 | | 86.5 +/− 3.9 | | | | 34.6 +/− 8.1 | −2.3 +/− 4.0 |
| K82N/N84S/T175R/R233E/E240N | 25-29 | 2 | | 59.2 +/− 8.2 | | | | 1.0 +/− 4.0 | −7.3 +/− 2.8 |
| Y[155]F/T175R/R233E/E240N | 24-28 | 2 | | 78.7 +/− 4.9 | | | | −5.7 +/− 2.8 | −4.2 +/− 3.7 |
| Y[155]F/K82N/N84S/R150Y/R170E/T175R | 28-30 | 2 | | 82.3 +/− 5.4 | | | | 64.6 +/− 7.4 | 41.4 +/− 7.7 |
| K82N/N84S/R150Y/R170E/T175R | 37-43 | 3 | | 79.3 +/− 4.2 | | | | 47.7 +/− 5.3 | 20.9 +/− 5.5 |
| R170E/T175R/E240N | 37-41 | 3 | | 66.6 +/− 5.9 | | | | 31.5 +/− 6 | 10.4 +/− 3.6 |
| R150Y/T175R/E240N | 24-28 | 2 | | 83.5 +/− 5.1 | | | | 36.7 +/− 8.9 | 20.0 +/− 6.7 |
| K63N/R150Y/R170E/T175R/R233E/E240N | 23-29 | 2 | | 84.5 +/− 3.1 | | | | 66.3 +/− 7.8 | 41.2 +/− 8.5 |
| K63N/K82N/N84S/R150Y/R170E/T175R/R233E | 22-28 | 2 | | 81.9 +/− 4.1 | | | | 62.2 +/− 8.2 | 28.6 +/− 8.0 |

### Example 8

### Determination of the Functional Cofactor Binding (K_{D-app}) of FIXa for its Cofactor, Factor VIIIa

The functional cofactor binding (K_{D-app}) of the FIXa variants for the cofactor Factor VIIIa (FVIIIa) in the presence or saturating substrate, Factor X (FX), was assessed indirectly in a fluorogenic assay by assaying for the activity of FXa, generated upon activation by FIXa, on the synthetic substrate Spectrafluor FXa. A range of FVIIIa concentrations were used to calculate the apparent kinetic rate constant (K_{D-app}) where the cofactor (FVIIIa) was in excess by at least a 1000-fold over the concentration of the activating protease (FIXa). The experiment was designed to be a variation of the assay described in Example 4 (Determination of the Catalytic Activity of FIXa for its Substrate, Factor X) where the cofactor (FVIIIa) at various concentrations is pre-incubated with FIXa in the presence of phospholipid vesicles forming the tenase (Xase) complex prior to assessing the catalytic activity with saturating levels of the substrate, FX. Briefly, activated and active site titrated FIXa was incubated in a calcium-containing buffer with phospholipid vesicles while separately recombinant FVIII is activated (to FVIIIa) with alpha-thrombin. The activity of alpha-thrombin was then quenched by the addition of a highly specific thrombin inhibitor, hirudin, prior to initiating the assay. FIXa variants were then mixed with various concentrations of FVIIIa to form the Xase complex and subsequently mixed with saturating concentrations of FX and the fluorescent substrate, Spectrafluor FXa (CH₃SO₂-D-CHA-Gly-Arg-AMC) to initiate the assay. The release of the free fluorophore, AMC (7-amino-4-methylcoumarin) following catalysis of Spectrafluor FXa by FXa was then assessed continuously over a time period, and the kinetic rate constants of the FIXa variants determined.

### A. Assay Protocol

For assays evaluating the kinetic rate of FX activation by FIXa in the presence of various FVIIIa concentrations and phospholipids, recombinant FVIII (Kogenate FS®; Bayer healthcare) was first resuspended in 1 mL of the provided diluent. The molar concentration of FVIII was then determined by absorbance at 280 nm using an extinction coefficient of 1.567 mg⁻¹ mL cm⁻¹ and a molecular weight of 163.6 kDa. The FIX variants were expressed, purified, activated and active site titrated as described in Examples 1-3, above. FIXa variants were then serially diluted to a concentration of 8 pM (4×) in a 1 mL volume of 1× Buffer A (20 mM Hepes/150 mM NaCl/5 mM CaCl₂)/0.1% BSA/0.1% PEG-8000, pH 7.4). In preparation for activation of FVIII to FVIIIa in the presence phospholipids, alpha-thrombin (Heamatologic Technologies, Inc.) and hirudin (American Diagnostica) were each diluted from the manufacturer's stock concentrations 1:100 in 1× Buffer A. Reconstituted FVIII was further diluted to a concentration of 1600 nM (4× of the top dose) in a 1.6 mL volume of 1× Buffer A containing 400 μM freshly resuspended phospholipids (75% phosphatidylcholine (PC)/25% phosphatidylserine (PS); PS/PC vesicles ˜120 nm in diameter; Avanti Polar Lipids). FVIII was activated to FVIIIa by mixing the above FVIII/PC/PS solution with a final concentration of 15 nM alpha-thrombin solutions followed by 15 minutes of incubation at 25° C. Activation reactions were subsequently quenched by the addition of hirudin to a final concentration of 150 nM for 5 min at 25° C. prior to initiating a dilution series of 1.5-fold in a 12-channel deep-well polypropylene plate with a final volume of 0.5 mL of the activated FVIIIa into 1× Buffer A containing 400 μM PC/PS vesicles. The final concentrations of FVIIIa (4×) were 1600 nM, 1066.7 nM, 711.1 nM, 474.1 nM, 316.1 nM, 210.7 nM, 140.5 nM, 93.6 nM, 62.43 nM, 41.6 nM. 27.8 nM and 0 nM for a 12-point assay or for an alternative 8-point assay with a 2-fold dilution series; 1600 nM, 600 nM, 400 nM, 200 nM, 100 nM, 50 nM, 25 nM and 0 nM. The dilution series of FVIIIa was subsequently mixed 1:1 with the 4×FIXa dilutions (12.5 μL each) in a 96-well half-area black assay plate according to a predefined plate map (4 FIXa variants/plate) and pre-incubated 15 min at 25° C. to form Xase complexes with varied concentrations of FVIIIa. Final 2× solutions (25 μL) were as follows: 4 pM FIXa variant, 1600-0 nM FVIIIa, 200 μM PC/PS vesicles, 7.5 nM alpha-thrombin (inhibited) and 75 nM hirudin.

A solution of 1000 nM (2×) active site titrated and DFP/EGR-cmk treated FX (see Example 2, above) was prepared in 20 mL of 1× Buffer A containing 1.0 mM Spectrafluor Xa substrate providing a sufficient volume for 4 assays. This represented a 2× saturating concentration of FX that would be at least 5-20-fold above the K_{M} values reported in Example 4, Table 25. Assay reactions were typically initiated using a BioMek FX liquid handling system programmed to dispense 25 μL of the FX/Spectrafluor Xa dilutions into 4 assay plates containing 25 μL of each FIXa variant and FVIIIa dilution (Xase complexes). The final concentrations of the reagents in the assay were as follows: 2 pM FIXa, 400-0 nM FVIIIa, 100 μM PC/PS vesicles, 0.5 mM Spectrafluor Xa, 3.8 nM alpha-thrombin (inhibited), 38 nM hirudin and FX at 500 nM. Reactions were monitored in a SpectraMax fluorescence plate reader for 30 min at 37° C. A standard curve of free AMC served as the conversion factor for RFU to μM in the subsequent data analysis calculations using a dose range that covered 0 μM to 100 μM AMC.

### B. Data Analysis

To determine functional affinity of FIXa variants for FVIIIa based on their catalytic activity, raw data collected with the SoftMax Pro application (Molecular Devices) were exported as .TXT files. Further non-linear data analyses were performed directly within the ActivityBase software package using the XE Runner data analysis module (IDBS Software). Data analyses were essentially as described in Example 4B with minor modifications. The Abase template was set up to automatically fit the parabolic reaction velocities (μM/sec²) of the tested FIXa variants at each FVIIIa concentration to the function of a standard rectangular hyperbola (i.e. Michaelis Menten equation) given by equation (1) to yield the fit values for Vₘₐₓ and K_{D-app}.

$$Reaction Velocity ( {\text{µ} \text{M} \text{/sec}}^{2} ) = \frac{{V}_{max} [ {S}_{0} ]}{{K}_{D - a p p} + [ {S}_{0} ]} \qquad Equation ( 1 )$$

Table 40 sets forth the functional affinity (K_{D-app}) for each of the FIXa variants assayed. Also assayed were recombinant wild-type FIXa (termed Catalyst Biosciences WT; generated as described above in Example 1), plasma purified FIXa (Haematologic Technologies, Inc.), and BeneFIX® (Coagulation Factor IX (Recombinant); Wyeth). Table 40 presents the results expressed as the kinetic constant for affinity, K_{D-app} (nM), and also as ratio of the functional affinity of the wild-type FIXa compared to that of the FIXa variant, wherein the functional affinity of each FIXa variant is defined by the K_{D-app} (nM) value for activation of the substrate, FX. Where the activity of the FIXa variant was compared to wild-type FIXa, it was compared to a recombinant wild-type FIXa polypeptide that was expressed and purified using the same conditions as used for the variant FIXa polypeptides to ensure that any differences in activity were the result of the mutation(s), and not the result of differences in, for example, post-translational modifications associated with different expression systems. Thus, the wild-type FIXa polypeptide used for comparison was the recombinant wild-type FIXa generated from cloning the FIX gene set forth in SEQ ID NO:1 and expressed from CHOX cells as a polypeptide with an amino acid sequence set forth in SEQ ID NO:3, as described in Example 1 (i.e., Catalyst Biosciences WT FIX polypeptide). The standard deviation (S.D.), coefficient of variation (as a percentage; % CV) and the number of assays performed (n) also are provided.

While some variants showed similar to wild-type affinities or nominal increases in K_{D-app} (e.g., FIXa-R318Y/R338E and FIXa-R318Y/R338E/R403E/E410N) several variants showed marked increases in functional affinity with greater than 6-10 fold increases in K_{D-app}. Variants with combinations of the R338E, T343R and E410N mutations showed the greatest improvements in functional affinity. For instance, FIXa-R338E/T343R, FIXa-R318Y/R338E/T343R/E410N, FIXa-R318Y/R338E/E410N, FIXa-Y155F/K247N/N249S/R318Y/R338E/T343R/R403E/E410N, FIXa-R338E/E410N and FIXa-K228N/247N/N249S/R318Y/R338E/T343R/E410N are among this group.

TABLE 40
Functional Cofactor Affinity of FIXa variants (K_{D-app})

| Mutation | Mutation | K_{D-app} | ±S.D. | | K_{D-WT}/ | |
|---|---|---|---|---|---|---|
| (Mature FIX Numbering) | (Chymotrypsin Numbering) | (nM) | (nM) | % CV | K_{D-mut} | n |
| BeneFIX Benefix ® Coagulation | BeneFIX Benefix ® Coagulation | 90.2 | 13.5 | 15% | 1.1 | 4 |
| FIX (T148A) | FIX (T[148]A) | | | | | |
| Plasma Purified FIXa | Plasma Purified FIXa | 101.6 | 5.8 | 6% | 0.9 | 3 |
| Catalyst Biosciences WT | Catalyst Biosciences WT | 95.5 | 4.6 | 5% | 1.0 | 2 |
| T148A | T[148]A | 79.7 | 27.1 | 34% | 1.2 | 2 |
| D104N/K106S/I251S | D[104]N/K[106]S/I86S | 305.5 | 119.5 | 39% | 0.3 | 2 |
| A262S | A95bS | 94.1 | 18.3 | 19% | 1.0 | 2 |
| E410N | E240N | 74.2 | 0.6 | 1% | 1.3 | 2 |
| E239N | E74N | 77.3 | 40.6 | 53% | 1.2 | 2 |
| T241N/H243S | T76N/H78S | 75.5 | 26.2 | 35% | 1.3 | 2 |
| S319N/L321S | S151N/L153S | 52.4 | 0.7 | 1% | 1.8 | 2 |
| R318E | R150E | 67.0 | 5.2 | 8% | 1.4 | 2 |
| R318Y | R150Y | 192.0 | 55.2 | 29% | 0.5 | 2 |
| R312Q | R143Q | 45.2 | 5.6 | 12% | 2.1 | 2 |
| R312A | R143A | 52.9 | 5.9 | 11% | 1.8 | 2 |
| R312Y | R143Y | 85.2 | 36.5 | 43% | 1.1 | 2 |
| R312L | R143L | 68.9 | 15.6 | 23% | 1.4 | 2 |
| V202Y | V38Y | 61.5 | 3.5 | 6% | 1.6 | 2 |
| D203Y | D39Y | 77.4 | 11.8 | 15% | 1.2 | 2 |
| A204M | A40M | 60.6 | 9.0 | 15% | 1.6 | 2 |
| K400A/R403A | K230A/R233A | 129.5 | 13.4 | 10% | 0.7 | 2 |
| K400E/R403E | K230E/R233E | 298.0 | 58.0 | 19% | 0.3 | 2 |
| R403E | R233E | 654.0 | 131.6 | 20% | 0.1 | 3 |
| K400A | K230A | 98.9 | 7.2 | 7% | 1.0 | 2 |
| K293A | K126A | 86.6 | 4.0 | 5% | 1.1 | 2 |
| R338E | R170E | 43.0 | 7.2 | 17% | 2.2 | 2 |
| R338E/R403E | R170E/R233E | 183.0 | 42.4 | 23% | 0.5 | 2 |
| R338E/E410N | R170E/E240N | 4.1 | 1.4 | 33% | 23.5 | 3 |
| R338E/R403E/E410N | R170E/R233E/E240N | 54.9 | 3.0 | 6% | 1.7 | 2 |
| R318Y/R338E/R403E | R150Y/R170E/R233E | 340.0 | 244.7 | 72% | 0.3 | 2 |
| R403E/E410N | R233E/E240N | 910.5 | 197.3 | 22% | 0.1 | 2 |
| R318Y/R338E/E410N | R150Y/R170E/E240N | 7.7 | 4.6 | 60% | 12.4 | 17 |
| D104N/K106S/R318Y/R338E/ | D[104]N/K[106]S/R150Y/R170E/ | 12.4 | n.d. | n.d. | 7.7 | 1 |
| E410N | E240N | | | | | |
| R318Y/R338E/R403E/E410N | R150Y/R170E/R233E/E240N | 47.0 | 12.4 | 26% | 2.0 | 12 |
| D104N/K106S/Y155F/R318Y/ | D[104]N/K[106]S/Y[155]F/ | 61.6 | n.d. | n.d. | 1.6 | 1 |
| R338E/R403E/E410N | R150Y/R170E/R233E/E240N | | | | | |
| K316N | K148N | 66.4 | 8.3 | 13% | 1.4 | 2 |
| H257E | H92E | 81.3 | 2.5 | 3% | 1.2 | 2 |
| E410S | E240S | 99.6 | 2.0 | 2% | 1.0 | 2 |
| N346D | N178D | 126.5 | 3.5 | 3% | 0.8 | 2 |
| N346Y | N178Y | 65.7 | n.d. | n.d. | 1.5 | 1 |
| Y345A | Y177A | 29.6 | 2.3 | 8% | 3.2 | 2 |
| T343R | T175R | 58.4 | 16.2 | 28% | 1.6 | 3 |
| T343R/Y345T | T175R/Y177T | 68.1 | n.d. | n.d. | 1.4 | 1 |
| R318Y/R338E | R150Y/R170E | 28.9 | n.d. | n.d. | 3.3 | 1 |
| Y259F/K265T/Y345T | Y94F/K98T/Y177T | 115.2 | n.d. | n.d. | 0.8 | 1 |
| K228N/I251S | K63N/I86S | 89.7 | 1.3 | 1% | 1.1 | 2 |
| Y155F/K228N/R318Y/R338E/ | Y[155]F/K63N/R150Y/R170E/ | 31.2 | 4.8 | 15% | 3.1 | 2 |
| R403E/E410N | R233E/E240N | | | | | |
| I251S/R318Y/R338E/R403E/ | I86S/R150Y/R170E/R233E/ | 62.7 | 0.6 | 1% | 1.5 | 2 |
| E410N | E240N | | | | | |
| D104N/K106S/I251S/R318Y/ | D[104]N/K[106]S/I86S/R150Y/ | 54.7 | 19.9 | 36% | 1.7 | 5 |
| R338E/R403E/E410N | R170E/R233E/E240N | | | | | |
| I251S/R318Y/R338E/E410N | I86S/R150Y/R170E/E240N | 5.7 | 1.1 | 20% | 16.7 | 3 |
| D104N/K106S/I251S/R318Y/ | D[104]N/K[106]S/I86S/R150Y/ | 12.4 | 1.1 | 9% | 7.7 | 2 |
| R338E/E410N | R170E/E240N | | | | | |
| K247N/N249S/R318Y/R338E/ | K82N/N84S/R150Y/R170E/ | 68.6 | 17.3 | 25% | 1.4 | 3 |
| R403E/E410N | R233E/E240N | | | | | |
| Y155F/K247N/N249S/R318Y/ | Y[155]F/K82N/N84S/R150Y/ | 45.8 | 4.6 | 10% | 2.1 | 7 |
| R338E/R403E/E410N | R170E/R233E/E240N | | | | | |
| A103N/N105S/K247N/N249S/ | A[103]N/N[105]S/K82N/N84S/ | 93.1 | 8.4 | 9% | 1.0 | 2 |
| R318Y/R338E/R403E/E410N | R150Y/R170E/R233E/E240N | | | | | |
| D104N/K106S/K247N/N249S/ | D[104]N/K[106]S/K82N/N84S/ | 87.4 | 10.3 | 12% | 1.1 | 2 |
| R318Y/R338E/R403E/E410N | R150Y/R170E/R233E/E240N | | | | | |
| Y155F/K247N/N249S/R318Y/ | Y[155]F/K82N/N84S/R150Y/ | 7.4 | n.d. | n.d. | 12.8 | 1 |
| R338E/E410N | R170E/E240N | | | | | |
| R318Y/R338E/R403E/E410S | R150Y/R170E/R233E/E240S | 53.1 | 10.4 | 20% | 1.8 | 3 |
| R318Y/R338E/E410S | R150Y/R170E/E240S | 6.8 | 0.2 | 3% | 14.1 | 3 |
| K228N/K247N/N249S | K63N/K82N/N84S | 113.0 | 0.0 | 0% | 0.8 | 2 |
| K228N/K247N/N249S/R318Y/ | K63N/K82N/N84S/R150Y/R170E/ | 100.5 | n.d. | n.d. | 0.9 | 1 |
| R338E/R403E/E410N | R233E/E240N | | | | | |
| R318Y/R338E/R403E/E410N/ | R150Y/R170E/R233E/E240N/ | 55.0 | n.d. | n.d. | 1.7 | 1 |
| T412V | T242V | | | | | |
| R318Y/R338E/E410N/T412V | R150Y/R170E/E240N/T242V | 8.9 | n.d. | n.d. | 10.7 | 1 |
| R318Y/R338E/N346D/R403E/ | R150Y/R170E/N178D/R233E/ | 109.7 | 44.3 | 40% | 0.9 | 2 |
| E410N | E240N | | | | | |
| K247N/N249S/N260S | K82N/N84S/N95S | 147.0 | 60.8 | 41% | 0.6 | 2 |
| Y155F/K247N/N249S/N260S | Y[155]F/K82N/N84S/N95S | 167.0 | 97.7 | 58% | 0.6 | 2 |
| D104N/K106S/K247N/N249S/ | D[104]N/K[106]S/K82N/N84S/ | 330.0 | 319.6 | 97% | 0.3 | 2 |
| N260S | N95S | | | | | |
| D104N/K106S/Y155F/K247N/ | D[104]N/K[106]S/Y[155]F/K82N/ | 142.0 | 73.5 | 52% | 0.7 | 2 |
| N249S/N260S | N84S/N95S | | | | | |
| K247N/N249S/N260S/R318Y/ | K82N/N84S/N95S/R150Y/R170E/ | 65.0 | 10.8 | 17% | 1.5 | 2 |
| R338E/R403E/E410N | R233E/E240N | | | | | |
| R318Y/R338E/T343R/R403E/ | R150Y/R170E/T175R/R233E/ | 14.5 | 4.0 | 28% | 6.6 | 7 |
| E410N | E240N | | | | | |
| R338E/T343R | R170E/T175R | 3.4 | 0.6 | 18% | 28.0 | 2 |
| T343R/N346Y | T175R/N178Y | 38.6 | n.d. | n.d. | 2.5 | 1 |
| R318Y/R338E/N346Y/R403E/ | R150Y/R170E/N178Y/R233E/ | 39.6 | n.d. | n.d. | 2.4 | 1 |
| E410N | E240N | | | | | |
| R318Y/R338E/T343R/N346Y/ | R150Y/R170E/T175R/N178Y/ | 15.6 | 0.1 | 1% | 6.1 | 2 |
| R403E/E410N | R233E/E240N | | | | | |
| T343R/N346D | T175R/N178D | 78.4 | n.d. | n.d. | 1.2 | 1 |
| R318Y/R338E/T343R/N346D/ | R150Y/R170E/T175R/N178D/ | 76.2 | n.d. | n.d. | 1.3 | 1 |
| R403E/E410N | R233E/E240N | | | | | |
| R318Y/R338E/T343R/E410N | R150Y/R170E/T175R/E240N | 6.1 | n.d. | n.d. | 15.7 | 1 |
| Y155F/R318Y/R338E/T343R/ | Y[155]F/R150Y/R170E/T175R/ | 7.4 | n.d. | n.d. | 12.8 | 1 |
| E410N | E240N | | | | | |
| R318Y/T343R/R403E/E410N | R150Y/T175R/R233E/E240N | 84.1 | 17.8 | 21% | 1.1 | 2 |
| R338E/T343R/R403E/E410N | R170E/T175R/R233E/E240N | 29.4 | n.d. | n.d. | 3.2 | 1 |
| Y155F/R338E/T343R/R403E/ | Y[155]F/R170E/T175R/R233E/ | 28.5 | n.d. | n.d. | 3.3 | 1 |
| E410N | E240N | | | | | |
| Y155F/K247N/N249S/R318Y/ | Y[155]F/K82N/N84S/R150Y/ | 15.3 | 1.3 | 9% | 6.3 | 3 |
| R338E/T343R/R403E/E410N | R170E/T175R/R233E/E240N | | | | | |
| K228N/K247N/N249S/R318Y/ | K63N/K82N/N84S/R150Y/R170E/ | 29.1 | 0.3 | 1% | 3.3 | 2 |
| R338E/T343R/R403E/E410N | T175R/R233E/E240N | | | | | |
| Y155F/K228N/K247N/N249S/R318Y/ | Y[155]F/K63N/K82N/N84S/R150Y/ | 37.0 | 5.7 | 16% | 2.6 | 2 |
| R338E/T343R/R403E/E410N | R170E/T175R/R233E/E240N | | | | | |
| Y155F/R338E/T343R/R403E | Y[155]F/R170E/T175R/R233E | 72.1 | n.d. | n.d. | 1.3 | 1 |
| R338E/T343R/R403E | R170E/T175R/R233E | 55.0 | n.d. | n.d. | 1.7 | 1 |
| R318Y/R338E/T343R/R403E/ | R150Y/R170E/T175R/R233E/ | 23.2 | n.d. | n.d. | 4.1 | 1 |
| E410S | E240S | | | | | |
| Y155F/K247N/N249S/R338E/ | Y[155]F/K82N/N84S/R170E/ | 15.4 | n.d. | n.d. | 6.2 | 1 |
| T343R | T175R | | | | | |
| Y155F/K247N/N249S/R318Y/ | Y[155]F/K82N/N84S/R150Y/ | 13.9 | n.d. | n.d. | 6.9 | 1 |
| R338E/T343R/E410N | R170E/T175R/E240N | | | | | |
| Y155F/K247N/N249S/R338E/ | Y[155]F/K82N/N84S/R170E/ | 24.9 | n.d. | n.d. | 3.8 | 1 |
| E410N | E240N | | | | | |
| K247N/N249S/R338E/T343R/ | K82N/N84S/R170E/T175R/ | 14.0 | n.d. | n.d. | 6.8 | 1 |
| E410N | E240N | | | | | |
| Y155F/R318Y/R338E/T343R | Y[155]F/R150Y/R170E/T175R | 8.4 | n.d. | n.d. | 11.3 | 1 |
| R318Y/R338E/T343R | R150Y/R170E/T175R | 9.8 | n.d. | n.d. | 9.7 | 1 |
| Y155F/K247N/N249S/R318Y/ | Y[155]F/K82N/N84S/R150Y/ | 14.0 | n.d. | n.d. | 6.8 | 1 |
| R338E/T343R | R170E/T175R | | | | | |
| K247N/N249S/R318Y/R338E/ | K82N/N84S/R150Y/R170E/ | 14.7 | n.d. | n.d. | 6.5 | 1 |
| T343R | T175R | | | | | |
| Y155F/R338E/T343R/E410N | Y[155]F/R170E/T175R/E240N | 8.5 | n.d. | n.d. | 11.2 | 1 |
| R338E/T343R/E410N | R170E/T175R/E240N | 7.5 | n.d. | n.d. | 12.8 | 1 |
| Y155F/R318Y/T343R/E410N | Y[155]F/R150Y/T175R/E240N | 38.0 | n.d. | n.d. | 2.5 | 1 |
| K228N/R150Y/R338E/T343R/ | K63N/R150Y/R170E/T175R/ | 17.5 | n.d. | n.d. | 5.4 | 1 |
| R403E/E410N | R233E/E240N | | | | | |
| K228N/247N/N249S/R318Y/ | K63N/K82N/N84S/R150Y/R170E/ | 7.8 | n.d. | n.d. | 12.2 | 1 |
| R338E/T343R/E410N | T175R/E240N | | | | | |

### Example 9

### Determination of the Clotting Activities of FIX Variants in Hemophilia B Plasma

Clotting activities for FIX variants were determine by an activated partial thromboplastin time (aPTT) assay in human hemophilia B plasma from a single donor with <1% clotting activity (George King Bio-Medical, Inc., Overland Park, Kans.) per the manufacturer's instructions. Briefly, the aPTT assay involves the recalcification of plasma in the presence of a blend of purified phospholipids (platelet substitute) and activators (kaolin and sulphatide). The aPTT assay was performed using the Dapttin®TC aPTT reagent (Technoclone GmbH, Vienna, Austria) essentially as described in the manufacturers' product insert with FIX variants spiked into the hemophilia B plasma at final concentrations of 100 nM, 10 nM or 1 nM FIX variant. Briefly, FIX variants were diluted to 1 μM in 1× Buffer A (20 mM Hepes/150 mM NaCl/0.5% BSA, pH 7.4) based on the active site titrated zymogen concentration (Example 2). FIX variants were subsequently serially diluted to 100 nM, 10 nM and 1 nM directly into citrated human hemophilia B plasma (George King Bio-Medical). A 100 μL volume of each FIX dilution in plasma was mixed with 100 μL of the Dapttin®TC aPTT reagent and incubated at 37° C. for 180 seconds. Coagulation was initiated by the addition of 100 μL of 25 mM calcium (Diagnostica Stago, Asnieres, France). Coagulation time in seconds was measured using a STArt4 instrument (Diagnostica Stago, Asnieres, France). Each experiment represents the average of two independent clotting time measurements, which typically showed <5% CV.

Table 41 sets forth the clotting activities for each of the FIX variants assayed. Also assayed were recombinant wild-type FIX (termed Catalyst Biosciences WT; generated as described above in Example 1), and BeneFIX® (Coagulation Factor IX (Recombinant); Wyeth). Table 41 presents the results expressed as the time to clot at each of the three tested FIX concentrations; 100 nM, 10 nM and 1 nM, wherein each

FIX concentration represents ˜100%, ˜10% and −1% of the normal concentration of FIX in pooled normal plasma (PNP). Under identical assay conditions, 100% PNP shows a clotting time of 31.3±2.0 seconds, whereas clotting times for 10% and 1% dilutions of PNP in hemophilia B plasma are 42.7±1.7 and 55.0±4.7 seconds, respectively (n=4). The time to clot for the hemophilia B plasma used in these analyses was evaluated 83.2±9.2 seconds (n=5). A number of tested variants demonstrated clotting times similar to or slightly prolonged compared to the wild-type FIXa, where wild-type FIXa polypeptide used for comparison was the recombinant wild-type FIXa expressed from CHOX cells as a polypeptide with an amino acid sequence set forth in SEQ ID NO:3, as described in Example 1 (i.e. Catalyst Biosciences WT FIX polypeptide). On the other hand, several variants showed significantly shortened clotting times. Among this group of variants are FIXa-R318Y/R338E/T343R, FIXa-R318Y/R338E/E410N, FIXa-R338E/T343R/E410N, FIXa-R318Y/R338E/T343R/E410N, FIXa-K247N/N249S/R338E/T343R/E410N and FIXa-K228N/247N/N249S/R318Y/R338E/T343R/E410N.

TABLE 41
Clotting Activity (aPTT) of FIX Variants in Hemophilia B Plasma

| Mutation | Mutation | aPTT | | aPTT | | aPTT | | |
|---|---|---|---|---|---|---|---|---|
| (Mature FIX | (Chymotrypsin | (100 nM) | | (10 nM) | | (1.0 nM) | | |
| Numbering) | Numbering) | (s) | ±S.D. | (s) | ±S.D. | (s) | ±S.D. | n |
| BeneFIX ® | BeneFIX ® | 35.2 | n.d. | 47.4 | n.d. | 63.5 | n.d. | 1 |
| Coagulation | Coagulation | | | | | | | |
| FIX (T148A) | FIX (T[148]A) | | | | | | | |
| Catalyst | Catalyst | 35.5 | n.d. | 46.9 | n.d. | 60.6 | n.d. | 1 |
| Biosciences | Biosciences | | | | | | | |
| WT | WT | | | | | | | |
| T148A | T[148]A | 33.2 | n.d. | 43.1 | n.d. | 59.2 | n.d. | 1 |
| R338E/R403E | R170E/R233E | 34.3 | n.d. | 46.2 | n.d. | 58.8 | n.d. | 1 |
| R338E/R403E/ | R170E/R233E/ | 35.6 | n.d. | 46.6 | n.d. | 57.1 | n.d. | 1 |
| E410N | E240N | | | | | | | |
| Y155F/R338E/ | Y[155]F/R170E/ | 31.1 | n.d. | 41.2 | n.d. | 52.6 | n.d. | 1 |
| R403E/E410N | R233E/E240N | | | | | | | |
| R318Y/R338E/ | R150Y/R170E/ | 41.7 | n.d. | 52.7 | n.d. | 68.4 | n.d. | 1 |
| R403E | R233E | | | | | | | |
| Y155F/R318Y/ | Y[155]F/R150Y/ | 38.6 | n.d. | 48.6 | n.d. | 64.1 | n.d. | 1 |
| R338E/R403E | R170E/R233E | | | | | | | |
| R318Y/R338E/ | R150Y/R170E/ | 21.2 | n.d. | 24.8 | n.d. | 34.3 | n.d. | 1 |
| E410N | E240N | | | | | | | |
| D104N/K106S/ | D[104]N/K[106]S/ | 24.5 | n.d. | 30.8 | n.d. | 40.0 | n.d. | 1 |
| R318Y/R338E/ | R150Y/R170E/ | | | | | | | |
| E410N | E240N | | | | | | | |
| R318Y/R403E/ | R150Y/R233E/ | 46.1 | n.d. | 61.7 | n.d. | 78.3 | n.d. | 1 |
| E410N | E240N | | | | | | | |
| Y155F/R318Y/ | Y[155]F/R150Y/ | 42.3 | n.d. | 57.1 | n.d. | 74.5 | n.d. | 1 |
| R403E/E410N | R233E/E240N | | | | | | | |
| R318Y/R338E/ | R150Y/R170E/ | 25.4 | 1.2 | 33.0 | 2.1 | 43.0 | 1.1 | 3 |
| R403E/E410N | R233E/E240N | | | | | | | |
| T343R | T175R | 41.3 | 2.1 | 53.3 | 2.9 | 67.2 | 6.2 | 2 |
| T343R/Y345T | T175R/Y177T | 46.8 | 2.8 | 56.3 | 9.6 | 75.5 | 1.8 | 2 |
| R318Y/R338E | R150Y/R170E | 26.7 | n.d. | 31.5 | n.d. | 45.3 | n.d. | 1 |
| Y155F/K228N/ | Y[155]F/K63N/ | 35.6 | n.d. | 45.1 | n.d. | 60.1 | n.d. | 1 |
| R318Y/R338E/ | R150Y/R170E/ | | | | | | | |
| R403E/E410N | R233E/E240N | | | | | | | |
| D104N/K106S/ | D[104]N/K[106]S/ | 36.0 | n.d. | 46.8 | n.d. | 61.8 | n.d. | 1 |
| I251S/R318Y/ | I86S/R150Y/ | | | | | | | |
| R338E/R403E/ | R170E/R233E/ | | | | | | | |
| E410N | E240N | | | | | | | |
| I251S/R318Y/ | I86S/R150Y/ | 28.0 | n.d. | 30.1 | n.d. | 40.7 | n.d. | 1 |
| R338E/E410N | R170E/E240N | | | | | | | |
| D104N/K106S/ | D[104]N/K[106]S/ | 25.0 | n.d. | 31.0 | n.d. | 43.1 | n.d. | 1 |
| I251S/R318Y/ | I86S/R150Y/ | | | | | | | |
| R338E/E410N | R170E/E240N | | | | | | | |
| K247N/N249S/ | K82N/N84S/ | 33.7 | n.d. | 43.8 | n.d. | 58.4 | n.d. | 1 |
| R318Y/R338E/ | R150Y/R170E/ | | | | | | | |
| R403E/E410N | R233E/E240N | | | | | | | |
| Y155F/K247N/ | Y[155]F/K82N/ | 34.1 | n.d. | 46.2 | n.d. | 62.4 | n.d. | 1 |
| N249S/R318Y/ | N84S/R150Y/ | | | | | | | |
| R338E/R403E/ | R170E/R233E/ | | | | | | | |
| E410N | E240N | | | | | | | |
| A103N/N105S/ | A[103]N/N[105]S/ | 36.1 | n.d. | 48.1 | n.d. | 62.6 | n.d. | 1 |
| K247N/N249S/ | K82N/N84S/ | | | | | | | |
| R318Y/R338E/ | R150Y/R170E/ | | | | | | | |
| R403E/E410N | R233E/E240N | | | | | | | |
| D104N/K106S/ | D[104]N/K[106]S/ | 34.8 | n.d. | 45.6 | n.d. | 59.3 | n.d. | 1 |
| Y155F/K247N/ | Y[155]F/K82N/ | | | | | | | |
| N249S/R318Y/ | N84S/R150Y/ | | | | | | | |
| R338E/R403E/ | R170E/R233E/ | | | | | | | |
| E410N | E240N | | | | | | | |
| K247N/N249S/ | K82N/N84S/ | 26.1 | n.d. | 34.3 | n.d. | 44.7 | n.d. | 1 |
| R318Y/R338E/ | R150Y/R170E/ | | | | | | | |
| E410N | E240N | | | | | | | |
| Y155F/K247N/ | Y[155]F/K82N/ | 24.0 | n.d. | 29.2 | n.d. | 41.1 | n.d. | 1 |
| N249S/R318Y/ | N84S/R150Y/ | | | | | | | |
| R338E/E410N | R170E/E240N | | | | | | | |
| R318Y/R338E/ | R150Y/R170E/ | 26.9 | n.d. | 34.7 | n.d. | 47.0 | n.d. | 1 |
| R403E/E410S | R233E/E240S | | | | | | | |
| K228N/K247N/ | K63N/K82N/ | 44.4 | n.d. | 57.2 | n.d. | 70.2 | n.d. | 1 |
| N249S | N84S | | | | | | | |
| D104N/K106S/ | D[104]N/K[106]S/ | 46.9 | n.d. | 60.0 | n.d. | 73.6 | n.d. | 1 |
| Y155F/K228N/ | Y[155]F/K63N/ | | | | | | | |
| K247N/N249S | K82N/N84S | | | | | | | |
| K228N/K247N/ | K63N/K82N/ | 35.3 | 5.1 | 46.1 | 8.0 | 60.6 | 8.9 | 2 |
| N249S/R318Y/ | N84S/R150Y/ | | | | | | | |
| R338E/R403E/ | R170E/R233E/ | | | | | | | |
| E410N | E240N | | | | | | | |
| D104N/K106S/ | D[104]N/K[106]S/ | 38.4 | n.d. | 50.1 | n.d. | 67.1 | n.d. | 1 |
| K228N/K247N/ | K63N/K82N/ | | | | | | | |
| N249S/R318Y/ | N84S/R150Y/ | | | | | | | |
| R338E/R403E/ | R170E/R233E/ | | | | | | | |
| E410N | E240N | | | | | | | |
| Y155F/K228N/ | Y[155]F/K63N/ | 34.9 | n.d. | 44.7 | n.d. | 59.1 | n.d. | 1 |
| K247N/N249S/ | K82N/N84S/ | | | | | | | |
| R318Y/R338E/ | R150Y/R170E/ | | | | | | | |
| R403E/E410N | R233E/E240N | | | | | | | |
| R318Y/R338E/ | R150Y/R170E/ | 28.7 | n.d. | 37.6 | n.d. | 47.6 | n.d. | 1 |
| R403E/E410N/ | R233E/E240N/ | | | | | | | |
| T412V | T242V | | | | | | | |
| R318Y/R338E/ | R150Y/R170E/ | 30.5 | n.d. | 40.6 | n.d. | 52.8 | n.d. | 1 |
| R403E/E410N/ | R233E/E240N/ | | | | | | | |
| T412A | T242A | | | | | | | |
| R318Y/R338E/ | R150Y/R170E/ | 25.5 | n.d. | 30.7 | n.d. | 40.3 | n.d. | 1 |
| E410N/T412V | E240N/T242V | | | | | | | |
| R318Y/R338E/ | R150Y/R170E/ | 42.5 | n.d. | 54.2 | n.d. | 68.9 | n.d. | 1 |
| N346D/R403E/ | N178D/R233E/ | | | | | | | |
| E410N | E240N | | | | | | | |
| Y155F/R318Y/ | Y[155]F/R150Y/ | 37.8 | n.d. | 48.9 | n.d. | 65.2 | n.d. | 1 |
| R338E/N346D/ | R170E/N178D/ | | | | | | | |
| R403E/E410N | R233E/E240N | | | | | | | |
| K247N/N249S/ | K82N/N84S/ | 44.7 | n.d. | 56.9 | n.d. | 75.7 | n.d. | 1 |
| N260S/R318Y/ | N95S/R150Y/ | | | | | | | |
| R338E/R403E/ | R170E/R233E/ | | | | | | | |
| E410N | E240N | | | | | | | |
| Y155F/K247N/ | Y[155]F/K82N/ | 49.3 | n.d. | 59.6 | n.d. | 75.5 | n.d. | 1 |
| N249S/N260S/ | N84S/N95S/ | | | | | | | |
| R318Y/R338E/ | R150Y/R170E/ | | | | | | | |
| R403E/E410N | R233E/E240N | | | | | | | |
| R318Y/R338E/ | R150Y/R170E/ | 23.7 | 2.7 | 29.7 | 3.3 | 39.7 | 6.5 | 4 |
| T343R/R403E/ | T175R/R233E/ | | | | | | | |
| E410N | E240N | | | | | | | |
| Y155F/R318Y/ | Y[155]F/R150Y/ | 26.2 | 3.6 | 32.0 | 3.9 | 42.4 | 1.8 | 2 |
| R338E/T343R/ | R170E/T175R/ | | | | | | | |
| R403E/E410N | R233E/E240N | | | | | | | |
| D104N/K106S/ | D[104]N/K[106]S/ | 27.3 | n.d. | 34.9 | n.d. | 48.0 | n.d. | 1 |
| R318Y/R338E/ | R150Y/R170E/ | | | | | | | |
| T343R/R403E/ | T175R/R233E/ | | | | | | | |
| E410N | E240N | | | | | | | |
| R338E/T343R | R170E/T175R | 27.9 | n.d. | 33.8 | n.d. | 45.1 | n.d. | 1 |
| T343R/N346Y | T175R/N178Y | 40.8 | 3.8 | 54.9 | 0.8 | 74.9 | 2.2 | 2 |
| R318Y/R338E/ | R150Y/R170E/ | 28.8 | n.d. | 41.0 | n.d. | 54.4 | n.d. | 1 |
| N346Y/R403E/ | N178Y/R233E/ | | | | | | | |
| E410N | E240N | | | | | | | |
| R318Y/R338E/ | R150Y/R170E/ | 24.5 | n.d. | 32.5 | n.d. | 41.7 | n.d. | 1 |
| T343R/N346Y/ | T175R/N178Y/ | | | | | | | |
| R403E/E410N | R233E/E240N | | | | | | | |
| T343R/N346D | T175R/N178D | 39.9 | 1.4 | 51.3 | 4.8 | 65.0 | 4.1 | 2 |
| R318Y/R338E/ | R150Y/R170E/ | 34.8 | n.d. | 45.1 | n.d. | 57.9 | n.d. | 1 |
| T343R/N346D/ | T175R/N178D/ | | | | | | | |
| R403E/E410N | R233E/E240N | | | | | | | |
| R318Y/R338E/ | R150Y/R170E/ | 41.2 | n.d. | 47.9 | n.d. | 61.9 | n.d. | 1 |
| Y345A/R403E/ | Y177A/R233E/ | | | | | | | |
| E410N | E240N | | | | | | | |
| Y155F/K247N/ | Y[155]F/K82N/ | 40.2 | n.d. | 51.6 | n.d. | 62.2 | n.d. | 1 |
| N249S/R318Y/ | N84S/R150Y/ | | | | | | | |
| R338E/R403E | R170E/R233E | | | | | | | |
| K247N/N249S/ | K82N/N84S/ | 42.0 | n.d. | 55.6 | n.d. | 70.3 | n.d. | 1 |
| R318Y/R338E/ | R150Y/R170E/ | | | | | | | |
| R403E | R233E | | | | | | | |
| K247N/N249S/ | K82N/N84S/ | 44.6 | 3.0 | 57.2 | 4.2 | 71.5 | 6.1 | 3 |
| R318Y/R403E/ | R150Y/R233E/ | | | | | | | |
| E410N | E240N | | | | | | | |
| Y155F/K247N/ | Y[155]F/K82N/ | 31.0 | n.d. | 42.1 | n.d. | 55.6 | n.d. | 1 |
| N249S/R338E/ | N84S/R170E/ | | | | | | | |
| R403E/E410N | R233E/E240N | | | | | | | |
| K247N/N249S/ | K82N/N84S/ | 32.7 | n.d. | 42.2 | n.d. | 56.2 | n.d. | 1 |
| R338E/R403E/ | R170E/R233E/ | | | | | | | |
| E410N | E240N | | | | | | | |
| R318Y/R338E/ | R150Y/R170E/ | 30.1 | n.d. | 37.9 | n.d. | 51.4 | n.d. | 1 |
| T343R/R403E | T175R/R233E | | | | | | | |
| Y155F/R318Y/ | Y[155]F/R150Y/ | 32.0 | n.d. | 41.5 | n.d. | 53.7 | n.d. | 1 |
| R338E/T343R/ | R170E/T175R/ | | | | | | | |
| R403E | R233E | | | | | | | |
| R318Y/R338E/ | R150Y/R170E/ | 24.7 | 2.9 | 27.2 | 2.9 | 36.5 | 3.8 | 5 |
| T343R/E410N | T175R/E240N | | | | | | | |
| Y155F/R318Y/ | Y[155]F/R150Y/ | 25.9 | 2.1 | 28.8 | 3.5 | 38.5 | 4.2 | 2 |
| R338E/T343R/ | R170E/T175R/ | | | | | | | |
| E410N | E240N | | | | | | | |
| R318Y/T343R/ | R150Y/T175R/ | 31.7 | n.d. | 43.3 | n.d. | 60.7 | n.d. | 1 |
| R403E/E410N | R233E/E240N | | | | | | | |
| Y155F/R318Y/ | Y[155]F/R150Y/ | 40.3 | n.d. | 52.0 | n.d. | 68.7 | n.d. | 1 |
| T343R/R403E/ | T175R/R233E/ | | | | | | | |
| E410N | E240N | | | | | | | |
| R338E/T343R/ | R170E/T175R/ | 25.5 | n.d. | 30.4 | n.d. | 41.9 | n.d. | 1 |
| R403E/E410N | R233E/E240N | | | | | | | |
| Y155F/R338E/ | Y[155]F/R170E/ | 27.5 | n.d. | 33.3 | n.d. | 42.3 | n.d. | 1 |
| T343R/R403E/ | T175R/R233E/ | | | | | | | |
| E410N | E240N | | | | | | | |
| Y155F/K247N/ | Y[155]F/K82N/ | 24.2 | 0.9 | 29.7 | 1.4 | 40.5 | 2.4 | 5 |
| N249S/R318Y/ | N84S/R150Y/ | | | | | | | |
| R338E/T343R/ | R170E/T175R/ | | | | | | | |
| R403E/E410N | R233E/E240N | | | | | | | |
| K247N/N249S/ | K82N/N84S/ | 28.7 | n.d. | 36.2 | n.d. | 50.2 | n.d. | 1 |
| R318Y/R338E/ | R150Y/R170E/ | | | | | | | |
| T343R/R403E/ | T175R/R233E/ | | | | | | | |
| E410N | E240N | | | | | | | |
| K228N/I251S/ | K63N/I86S/ | 34.5 | n.d. | 44.9 | n.d. | 58.2 | n.d. | 1 |
| R318Y/R338E/ | R150Y/R170E/ | | | | | | | |
| R403E/E410N | R233E/E240N | | | | | | | |
| Y155F/K228N/ | Y[155]F/K63N/ | 34.5 | n.d. | 46.5 | n.d. | 60.3 | n.d. | 1 |
| I251S/R318Y/ | I86S/R150Y/ | | | | | | | |
| R338E/R403E/ | R170E/R233E/ | | | | | | | |
| E410N | E240N | | | | | | | |
| N260S/R318Y/ | N95S/R150Y/ | 31.4 | n.d. | 41.1 | n.d. | 55.4 | n.d. | 1 |
| R338E/T343R/ | R170E/T175R/ | | | | | | | |
| R403E/E410N | R233E/E240N | | | | | | | |
| Y155F/N260S/ | Y[155]F/N95S/ | 35.3 | 0.6 | 45.3 | 2.5 | 59.1 | 3.2 | 2 |
| R318Y/R338E/ | R150Y/R170E/ | | | | | | | |
| T343R/R403E/ | T175R/R233E/ | | | | | | | |
| E410N | E240N | | | | | | | |
| K228N/K247N/ | K63N/K82N/ | 28.0 | 2.0 | 35.5 | 3.9 | 47.7 | 6.0 | 8 |
| N249S/R318Y/ | N84S/R150Y/ | | | | | | | |
| R338E/T343R/ | R170E/T175R/ | | | | | | | |
| R403E/E410N | R233E/E240N | | | | | | | |
| Y155F/K228N/ | Y[155]F/K63N/ | 30.7 | 2.3 | 40.6 | 2.0 | 53.5 | 2.5 | 2 |
| K247N/N249S/ | K82N/N84S/ | | | | | | | |
| R318Y/R338E/ | R150Y/R170E/ | | | | | | | |
| T343R/R403E/ | T175R/R233E/ | | | | | | | |
| E410N | E240N | | | | | | | |
| Y155F/R338E/ | Y[155]F/R170E/ | 29.8 | n.d. | 37.9 | n.d. | 50.1 | n.d. | 1 |
| T343R/R403E | T175R/R233E | | | | | | | |
| R338E/T343R/ | R170E/T175R/ | 29.4 | n.d. | 37.0 | n.d. | 49.8 | n.d. | 1 |
| R403E | R233E | | | | | | | |
| Y155F/R338E/ | Y[155]F/R170E/ | 28.3 | n.d. | 33.3 | n.d. | 44.4 | n.d. | 1 |
| T343R/R403E/ | T175R/R233E/ | | | | | | | |
| E410S | E240S | | | | | | | |
| Y155F/N260S/ | Y[155]F/N95S/ | 40.5 | n.d. | 52.9 | n.d. | 70.1 | n.d. | 1 |
| R338E/T343R/ | R170E/T175R/ | | | | | | | |
| R403E | R233E | | | | | | | |
| Y155F/I251S/ | Y[155]F/I86S/ | 31.9 | n.d. | 40.1 | n.d. | 54.5 | n.d. | 1 |
| R338E/T343R/ | R170E/T175R/ | | | | | | | |
| R403E | R233E | | | | | | | |
| R318Y/R338E/ | R150Y/R170E/ | 27.4 | n.d. | 34.0 | n.d. | 43.3 | n.d. | 1 |
| T343R/R403E/ | T175R/R233E/ | | | | | | | |
| E410S | E240S | | | | | | | |
| Y155F/K247N/ | Y[155]F/K82N/ | 43.2 | n.d. | 58.6 | n.d. | 74.2 | n.d. | 1 |
| N249S/T343R/ | N84S/T175R/ | | | | | | | |
| R403E | R233E | | | | | | | |
| Y155F/K247N/ | Y[155]F/K82N/ | 32.5 | n.d. | 41.4 | n.d. | 55.4 | n.d. | 1 |
| N249S/R318Y/ | N84S/R150Y/ | | | | | | | |
| R338E/T343R/ | R170E/T175R/ | | | | | | | |
| R403E | R233E | | | | | | | |
| K247N/N249S/ | K82N/N84S/ | 30.8 | 4.2 | 39.1 | 6.9 | 52.5 | 9.1 | 2 |
| R318Y/R338E/ | R150Y/R170E/ | | | | | | | |
| T343R/R403E | T175R/R233E | | | | | | | |
| Y155F/K247N/ | Y[155]F/K82N/ | 27.3 | n.d. | 34.9 | n.d. | 47.7 | n.d. | 1 |
| N249S/R338E/ | N84S/R170E/ | | | | | | | |
| T343R/R403E/ | T175R/R233E/ | | | | | | | |
| E410N | E240N | | | | | | | |
| K247N/N249S/ | K82N/N84S/ | 28.2 | n.d. | 35.1 | n.d. | 47.3 | n.d. | 1 |
| R338E/T343R/ | R170E/T175R/ | | | | | | | |
| R403E/E410N | R233E/E240N | | | | | | | |
| Y155F/K247N/ | Y[155]F/K82N/ | 29.6 | n.d. | 37.4 | n.d. | 48.7 | n.d. | 1 |
| N249S/R318Y/ | N84S/R150Y/ | | | | | | | |
| R338E | R170E | | | | | | | |
| Y155F/K247N/ | Y[155]F/K82N/ | 39.6 | n.d. | 49.7 | n.d. | 65.0 | n.d. | 1 |
| N249S/R318Y/ | N84S/R150Y/ | | | | | | | |
| T343R | T175R | | | | | | | |
| Y155F/K247N/ | Y[155]F/K82N/ | 52.2 | n.d. | 67.9 | n.d. | 79.9 | n.d. | 1 |
| N249S/R318Y/ | N84S/R150Y/ | | | | | | | |
| R403E | R233E | | | | | | | |
| Y155F/K247N/ | Y[155]F/K82N/ | 32.9 | n.d. | 43.8 | n.d. | 55.8 | n.d. | 1 |
| N249S/R318Y/ | N84S/R150Y/ | | | | | | | |
| E410N | E240N | | | | | | | |
| Y155F/K247N/ | Y[155]F/K82N/ | 39.2 | n.d. | 50.4 | n.d. | 62.6 | n.d. | 1 |
| N249S/R338E/ | N84S/R170E/ | | | | | | | |
| R403E | R233E | | | | | | | |
| Y155F/K247N/ | Y[155]F/K82N/ | 27.4 | n.d. | 31.5 | n.d. | 41.8 | n.d. | 1 |
| N249S/R338E/ | N84S/R170E/ | | | | | | | |
| T343R | T175R | | | | | | | |
| Y155F/K247N/ | Y[155]F/K82N/ | 28.7 | 0.4 | 32.7 | 0.1 | 41.8 | 0.9 | 2 |
| N249S/R318Y/ | N84S/R150Y/ | | | | | | | |
| R338E/T343R/ | R170E/T175R/ | | | | | | | |
| E410N | E240N | | | | | | | |
| K247N/N249S/ | K82N/N84S/ | 28.0 | 0.8 | 32.7 | 0.8 | 42.4 | 0.3 | 2 |
| R318Y/R338E/ | R150Y/R170E/ | | | | | | | |
| T343R/E410N | T175R/E240N | | | | | | | |
| Y155F/K247N/ | Y[155]F/K82N/ | 38.9 | n.d. | 50.4 | n.d. | 65.5 | n.d. | 1 |
| N249S/R318Y/ | N84S/R150Y/ | | | | | | | |
| T343R/R403E/ | T175R/R233E/ | | | | | | | |
| E410N | E240N | | | | | | | |
| K247N/N249S/ | K82N/N84S/ | 35.9 | 4.2 | 46.6 | 6.0 | 60.9 | 7.8 | 2 |
| R318Y/T343R/ | R150Y/T175R/ | | | | | | | |
| R403E/E410N | R233E/E240N | | | | | | | |
| Y155F/K247N/ | Y[155]F/K82N/ | 27.1 | 1.9 | 31.8 | 2.0 | 41.2 | 0.8 | 2 |
| N249S/R338E/ | N84S/R170E/ | | | | | | | |
| E410N | E240N | | | | | | | |
| Y155F/K247N/ | Y[155]F/K82N/ | 44.3 | n.d. | 60.7 | n.d. | 75.5 | n.d. | 1 |
| N249S/R318Y/ | N84S/R150Y/ | | | | | | | |
| T343R/R403E | T175R/R233E | | | | | | | |
| K247N/N249S/ | K82N/N84S/ | 45.3 | n.d. | 57.5 | n.d. | 75.7 | n.d. | 1 |
| R318Y/T343R/ | R150Y/T175R/ | | | | | | | |
| R403E | R233E | | | | | | | |
| Y155F/K247N/ | Y[155]F/K82N/ | 44.9 | 0.1 | 52.5 | 3.7 | 64.9 | 0.5 | 2 |
| N249S/R318Y/ | N84S/R150Y/ | | | | | | | |
| T343R/E410N | T175R/E240N | | | | | | | |
| K247N/N249S/ | K82N/N84S/ | 42.7 | n.d. | 50.2 | n.d. | 64.6 | n.d. | 1 |
| R318Y/T343R/ | R150Y/T175R/ | | | | | | | |
| E410N | E240N | | | | | | | |
| Y155F/K247N/ | Y[155]F/K82N/ | 31.1 | n.d. | 40.9 | n.d. | 56.2 | n.d. | 1 |
| N249S/R338E/ | N84S/R170E/ | | | | | | | |
| T343R/R403E | T175R/R233E | | | | | | | |
| K247N/N249S/ | K82N/N84S/ | 32.0 | n.d. | 43.2 | n.d. | 56.1 | n.d. | 1 |
| R338E/T343R/ | R170E/T175R/ | | | | | | | |
| R403E | R233E | | | | | | | |
| Y155F/K247N/ | Y[155]F/K82N/ | 28.5 | n.d. | 32.2 | n.d. | 45.9 | n.d. | 1 |
| N249S/R338E/ | N84S/R170E/ | | | | | | | |
| T343R/E410N | T175R/E240N | | | | | | | |
| K247N/N249S/ | K82N/N84S/ | 25.1 | 3.9 | 29.9 | 5.0 | 41.1 | 8.0 | 2 |
| R338E/T343R/ | R170E/T175R/ | | | | | | | |
| E410N | E240N | | | | | | | |
| Y155F/K247N/ | Y[155]F/K82N/ | 36.7 | n.d. | 49.3 | n.d. | 65.4 | n.d. | 1 |
| N249S/T343R/ | N84S/T175R/ | | | | | | | |
| R403E/E410N | R233E/E240N | | | | | | | |
| Y155F/R318Y/ | Y[155]F/R150Y/ | 27.4 | 1.0 | 31.4 | 1.7 | 40.7 | 0.4 | 2 |
| R338E/T343R | R170E/T175R | | | | | | | |
| R318Y/R338E/ | R150Y/R170E/ | 20.5 | n.d. | 24.3 | n.d. | 32.2 | n.d. | 1 |
| T343R | T175R | | | | | | | |
| Y155F/R318Y/ | Y[155]F/R150Y/ | 43.4 | n.d. | 56.1 | n.d. | 71.3 | n.d. | 1 |
| T343R/R403E | T175R/R233E | | | | | | | |
| Y155F/T343R/ | Y[155]F/T175R/ | 36.1 | n.d. | 47.5 | n.d. | 63.0 | n.d. | 1 |
| R403E/E410N | R233E/E240N | | | | | | | |
| Y155F/K247N/ | Y[155]F/K82N/ | 28.0 | 1.4 | 32.9 | 0.8 | 42.6 | 0.4 | 2 |
| N249S/R318Y/ | N84S/R150Y/ | | | | | | | |
| R338E/T343R | R170E/T175R | | | | | | | |
| K247N/N249S/ | K82N/N84S/ | 27.4 | 1.2 | 32.7 | 0.2 | 42.4 | 3.1 | 2 |
| R318Y/R338E/ | R150Y/R170E/ | | | | | | | |
| T343R | T175R | | | | | | | |
| Y155F/K247N/ | Y[155]F/K82N/ | 36.2 | 4.5 | 44.8 | 5.9 | 54.4 | 4.2 | 5 |
| N249S/T343R/ | N84S/T175R/ | | | | | | | |
| E410N | E240N | | | | | | | |
| Y155F/K247N/ | Y[155]F/K82N/ | 47.2 | n.d. | 60.7 | n.d. | 74.2 | n.d. | 1 |
| N249S/R403E/ | N84S/R233E/ | | | | | | | |
| E410N | E240N | | | | | | | |
| Y155F/R338E/ | Y[155]F/R170E/ | 24.9 | 4.4 | 27.5 | 4.4 | 34.9 | 4.4 | 4 |
| T343R/E410N | T175R/E240N | | | | | | | |
| R338E/T343R/ | R170E/T175R/ | 19.8 | n.d. | 23.9 | n.d. | 34.7 | n.d. | 1 |
| E410N | E240N | | | | | | | |
| Y155F/R318Y/ | Y[155]F/R150Y/ | 41.3 | 5.7 | 49.5 | 6.0 | 63.4 | 6.0 | 2 |
| T343R/E410N | T175R/E240N | | | | | | | |
| R318Y/T343R/ | R150Y/T175R/ | 34.5 | n.d. | 44.8 | n.d. | 61.0 | n.d. | 1 |
| E410N | E240N | | | | | | | |
| K228N/R318Y/ | K63N/R150Y/ | 23.4 | n.d. | 28.8 | n.d. | 38.9 | n.d. | 1 |
| R338E/T343R/ | R170E/T175R/ | | | | | | | |
| R403E/E410N | R233E/E240N | | | | | | | |
| K228N/K247N/ | K63N/K82N/ | 28.6 | n.d. | 37.3 | n.d. | 47.9 | n.d. | 1 |
| N249S/R318Y/ | N84S/R150Y/ | | | | | | | |
| R338E/T343R/ | R170E/T175R/ | | | | | | | |
| R403E | R233E | | | | | | | |
| K228N/247N/ | K63N/K82N/ | 21.4 | n.d. | 25.8 | n.d. | 34.3 | n.d. | 1 |
| N249S/R318Y/ | N84S/R150Y/ | | | | | | | |
| R338E/T343R/ | R170E/T175R/ | | | | | | | |
| E410N | E240N | | | | | | | |
| K228N/K247N/ | K63N/K82N/ | 35.4 | n.d. | 44.0 | n.d. | 61.4 | n.d. | 1 |
| N249S/R318Y/ | N84S/R150Y/ | | | | | | | |
| T343R/R403E/ | T175R/R233E/ | | | | | | | |
| E410N | E240N | | | | | | | |

### Example 10

### A. AAV Capsid Selection

Capsid libraries, containing barcodes for tracking and identification, were generated, and library diversity was validated by DNA sequencing. Unique barcodes were used to track variant enrichment over passage rounds via high-throughput sequencing (HTS) using three methods: high throughput sequencing of the barcodes using a MiSeq sequencer (Illumina); standard PacBio real-time sequencing (Pacbio) of the capsids including the barcodes; and Sanger sequencing of the capsids including the barcodes. Capsid genes from various AAV wild-type serotypes and previously described variants were shuffled using DNase shuffling to create highly complex and functional capsid libraries (see, e.g., Stemmer (1994) Proc. Natl. Acad. Sci. U.S.A. 91:10747-10751). Capsid variants that demonstrated improved transduction efficiency in human islet cells were selected. Details of the generation and selection of the capsid proteins are described in International PCT application No. PCT/US2019/025026, published as WO2019/191701.

### B. AAV Capsid Transduction Efficiency in Liver

Transduction efficiency of each of the rAAVs packaged with the variant capsids (e.g., capsids whose protein and encoding nucleic acid sequences are set forth in SEQ ID NOs: 418-423) also was assessed in vivo. Balb/C SCID mice were injected in the tail vein with 2 E+10 viral genomes (vgs) of a luciferase rAAV vector packaged with the new variant capsids set forth in SEQ ID NOS: 418-423, and previously characterized capsids AAV8 and AAV-DJ (sold by CellBiolabs, Inc.; SEQ ID NOs: 424-427) which show tropism for the liver. The results show that for all capsid variants tested, the majority of the AAV capsids targeted to the liver, as assessed by live imaging for luciferase. Luciferase expression also was analyzed in several mouse organs, post-mortem, thirty-four days after injection. The results show that luciferase was expressed highest in liver compared to other organs. Vector genomes were quantified in the organs using qPCR. Vector genome copies above background were detected only for liver samples. Mice injected with the capsid variant, whose protein and nucleic acid sequence is set forth in SEQ ID NOs: 418 and 421(KP1), respectively, contained higher vector copy numbers than the mice that had received the previously characterized AAV-DJ packaged vector (nucleic acid and protein sequences of the capsid set forth in SEQ ID NOs:424 and 425, respectively) and AAV-8 packaged vector. Mice injected with the capsid variant, designated KP-3, whose amino acid and nucleic acid sequences are set forth in SEQ ID NOs:420 and 423, respectively, had significantly more vector genomes (vgs) in their livers than AAV-8 injected mice, and had similar levels to the DJ injected mice. Mice injected with the capsid variant, designated KP-2, whose amino acid and nucleic acid sequences are set forth in SEQ ID NO: 419 and SEQ ID NO:422, respectively, had similar levels of rAAV genomes as AAV8 injected mice and fewer than DJ injected mice.

Transduction efficiency of hepatocytes with rAAV packaged with the capsid, designated KP1 (SEQ ID NOs: 418 and 421) was assessed in xenograft liver models. AAV-KP1 transduced human and mouse hepatocytes at higher levels than the AAV-DJ (SEQ ID NOs:424 and 425). Capsid variants that demonstrated improved transduction efficiency of hepatocytes for rodents and humans and in vivo in the liver are those packaged in the capsids designated KP1, KP2, and KP3, whose nucleic acid and protein sequences are set forth in SEQ ID NOs:418-423. Each of the capsids were analyzed to determine the prevalence of various serotypes in the sequence. The results show that the capsid KP1 (SEQ ID NOs:418 and 421) contains fragments from 7 of the 8 parental serotypes, and the capsids designated KP2 and KP3 (SEQ ID NOs:419, 420, 422, and 423) contain fragments from 6 parental serotypes. The capsid designated KP1 contains stretches of several different parental sequences at the N-terminus, while the capsids designated KP2 and KP3 each contain a less diverse N-terminus. The capsids designated KP1 and KP3 are enriched for AAV2 in the N-terminus, and share common residues from AAV8 at the C-terminus. In each of the capsids designated KP1, KP2 and KP3, most of the C-terminus was derived from AAV3B. KP1 and KP3 share 92% overall sequence identity with AAV3B; KP3 shares 95% overall amino acid sequence identity with AAV3B. All three of these capsids share residues that are unique to AAV1 and AAV6 in the sequence stretch between amino acids 225 and 267, and all contain arginine at position 597, which was previously characterized as a heparin sulfate proteoglycan binding site (Lerch et al. (2012) Virology 423:6-13). Table 42, below, summarizes shared residues among these capsids that exhibit the improved transduction of hepatocytes. The table shows shared amino acid residues among AAV capsid variants designated KP1, KP2 and KP3. Numbering of residues of AAV3B, KP1, KP2, and KP3 is with reference to alignment with SEQ ID NO:418 (KP1). Shown are residues that are different from AAV3B and that are shared among at least two of the variants shown. Amino acids shared among all three variant capsids are highlighted in bold. A blank field indicates a residue identical to that of AAV3B. A missing amino acid is indicated (-). HVAR is the hypervariable region. Surface exposed residues on the VP3 capsid protein are marked (*)

TABLE 42

| Position | Capsid protein/HVAR | AAV3B | KP1 | KP2 | KP3 |
|---|---|---|---|---|---|
| 14 | VP1 | N | T | | T |
| 21 | VP1 | E | Q | | Q |
| 24 | VP1 | A | K | D | K |
| 29 | VP1 | V | P | A | P |
| 31 | VP1 | Q | P | K | P |
| 34 | VP1 | A | P | | P |
| 35 | VP1 | N | A | | A |
| 36 | VP1 | Q | E | | E |
| 37 | VP1 | Q | R | | R |
| 39 | VP1 | Q | K | | K |
| 41 | VP1 | N | D | D | D |
| 42 | VP1 | R | S | G | S |
| 56 | VP1 | G | | F | F |
| 67 | VP1 | E | A | A | |
| 92 | VP1 | K | R | R | R |
| 125 | VP1 | I | V | V | V |
| 135 | VP1 | A | P | G | G |
| 147 | VP1, VP2 | D | E | E | E |
| 148 | VP1, VP2 | Q | H | H | P |
| 151 | VP1, VP2 | Q | V | V | |
| 159 | VP1, VP2 | V | T | I | I |
| 164 | VP1, VP2 | K | Q | Q | Q |
| 180 | VP1, VP2 | E | D | D | |
| 196 | VP1, VP2 | T | S | S | S |
| 197 | VP1, VP2 | S | G | G | G |
| 200 | VP1, VP2 | S | T | T | P |
| 205 | VP1, VP2, VP3 | S | A | T | A |
| 224 | VP1, VP2, VP3 | S | A | A | A |
| 233 | VP1, VP2, VP3 | Q | T | T | T |
| 263* | VP1, VP2, VP3 (HVR I) | Q | A | A | A |
| 265* | VP1, VP2, VP3 (HVR I) | — | T | T | T |
| 311 | VP1, VP2, VP3 | K | R | R | R |
| 313 | VP1, VP2, VP3 | S | N | N | N |
| 327* | VP1, VP2, VP3 (HVR II) | D | E | E | E |
| 331* | VP1, VP2, VP3 (HVR II) | T | K | | K |
| 373 | VP1, VP2, VP3 | V | I | I | I |
| 649 | VP1, VP2, VP3 | M | L | | L |
| 658* | VP1, VP2, VP3 | N | D | | D |
| 664* | VP1, VP2, VP3 | S | N | | N |
| 668* | VP1, VP2, VP3 | F | L | | L |
| 669* | VP1, VP2, VP3 | A | N | | N |
| 707* | VP1, VP2, VP3 (HVR IX) | N | Y | | Y |
| 710* | VP1, VP2, VP3 (HVR IX) | V | T | | T |
| 711* | VP1, VP2, VP3 (HVR IX) | N | S | | S |
| 715* | VP1, VP2, VP3 (HVR IX) | T | A | | A |
| 717* | VP1, VP2, VP3 (HVR IX) | D | N | | N |
| 719* | VP1, VP2, VP3 (HVR IX) | N | E | | E |

### Example 11

### rAAV-Modified FIX (Designated CB2679) Vector Construction, Production and Titration

In all examples, except where noted, the mature form of this FIX has the sequence set forth in SEQ ID NO:394. Where noted, the replacements are in a FIX allele with T148A (SEQ ID NO:490). The amino acid sequence of the mature form of FIX encoded by the wild-type allele with the T148A replacement is set forth in SEQ ID NO:20 and also SEQ ID NO:489. The nucleic acid sequence of the modified mature FIX allele is set forth in SEQ ID NO:486. The T148A replacement does not alter activity of FIX or modified FIX. The activities of these the modified FIX polypeptides are substantially the same.

### A. Cloning of the FIX Gene

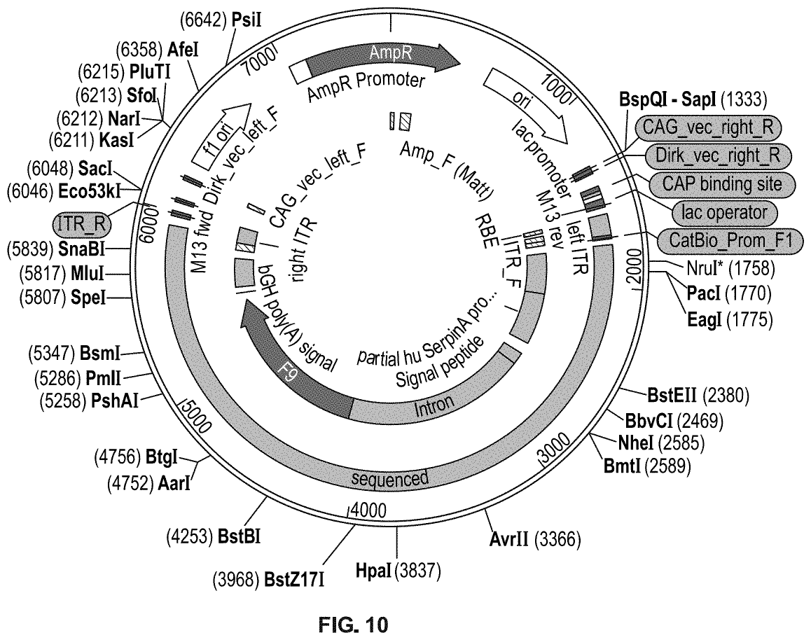

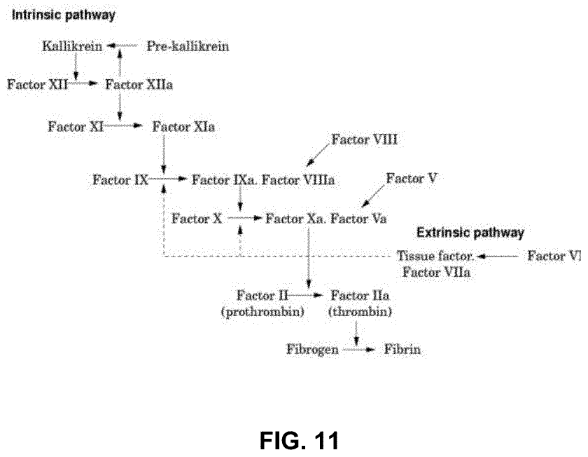

The nucleic acid encoding the 461 amino acid wild-type FIX precursor polypeptide (set forth in SEQ ID NO: 2) with a portion of the first FIX intron (nucleotides 2691..4128 of SEQ ID NO: 451; collectively the FIX “minigene” or FIX-GT, for FIX Gene Therapy) cloned immediately downstream of the first amino acid following the FIX signal sequence was cloned into the rAAV vector (set forth in SEQ ID NO:555). The rAAV vector contains an apolipoprotein E locus control region (ApoE-HCR) enhancer (set forth in SEQ ID NO: 438), the human Serpin A alpha-1 antitrypsin liver specific promoter (hAAT) (set forth in SEQ ID NO: 440), and a bovine growth hormone polyA (pA; set forth in SEQ ID NO: 443) (see, FIG. 10). Viral DNA packaging sequences (inverted terminal repeats [ITRs]) flank the enhancer-promoter-FIX-pA cassette.

Site-directed mutagenesis generated the Padua FIX, which contains the R338L mutation (SEQ ID NO: 484), and the modified FIX containing the R318Y, R338E and T343R mutations (SEQ ID NO:394 or 490 (T148A allele)) using the QuikChange II kit according to manufacturer's instructions. In these examples modified FIX refers to the variant that contains the replacements R318Y/R338E/T343R. The complete sequence of the rAAV vector containing wild-type hFIX, modified hFIX, and Padua FIX are set forth in SEQ IDS NOs: 447, 448 and 449, respectively. Additional vectors containing wild-type hFIX, modified hFIX, and Padua FIX are set forth in SEQ IDS NOs: 450-455.

The hFIX minigene encodes a FIX precursor protein that contains a 28 amino acid signal peptide (nucleotides 2604-2687 of any of SEQ ID NOs: 447, 448 and 449; or nucleotides 1-84 of SEQ ID NO: 1) followed by an 18 amino acid propeptide (nucleotides 2688-2690 and 4129-4179 of any of SEQ ID NOs: 447, 448 and 449; or nucleotides 85-138 of SEQ ID NO: 1), and the 415 amino acid mature human factor IX zymogen (nucleotides 4180-5427 of any of SEQ ID NOs: 447, 448 and 449; or nucleotides 139-1383 of SEQ ID NO: 1). The FIX mini-intron (1438 bp spanning nucleotides 2691-4128 of any of SEQ ID NOs: 447, 448 and 449) is inserted after the first codon of the propeptide, and before the second codon (e.g., immediately after nucleotide 87 of SEQ ID NO: 1); it is spliced out upon functional expression. The FIX mini-intron increases transgene expression by 10-fold when used in mouse liver (see, e.g., Kurachi et al. J. Biol. Chem 270(10):5276-5281 (1995)). With a total size of 4.4 kb including the ITRs, the vector is well within the range that can be efficiently packaged in an AAV capsid.

B. rAAV Vector Production

rAAV vectors (SEQ ID NOs: 447, 449 and 448), prepared as detailed above, respectively encoding wild-type FIX (mature sequence set forth SEQ ID NO: 3), Padua FIX (mature sequence set forth in SEQ ID NO:484), and another exemplary modified human FIX (mature sequence set forth in SEQ ID NO:394, designated CB2679 elsewhere herein, and referred to below as modified FIX), packaged with the capsid designated KP1, were generated using triple plasmid calcium phosphate transfection (large scale preparation) or Polyethylenimine (PEI) (small scale preparation) transfection protocol of HEK-293T/17 cells. rAAV was purified from the cell lysates by two rounds of cesium chloride (CsCl) gradient ultracentrifugation purification (see e.g., Grimm et al, J. of Virology 80(1):426-439 (2006); Pekrun et al., JCI insight 4(22):e131610 (2019)).

For large-scale production, HEK-293T cells (ATCC #CRL-3216, Manassas, Va.) were seeded and expanded for 2 days prior to transfection in T225 flasks (Corning Inc., Corning, N.Y.) using 40×225 cm² flasks for each prep. The cells were then transfected using a standard calcium phosphate-based protocol or with DNA-OptiMEM-PEI mixture (Polyethylenimine, linear MW 25000, transfection grade, PEI 25K, Polysciences, Inc., #23966-1, Warrington, Pa.) with 75 μg of plasmid DNA per flask, consisting of an equimolar mixture of: a) hFIX-encoding rAAV vector plasmid encoding the modified FIX (mature sequence set forth in SEQ ID NO:394), Padua FIX (mature sequence set forth in SEQ ID NO:484), or wild type FIX (mature sequence set forth in SEQ ID NO:3), b) AAV serotype-specific packaging plasmid (e.g., SEQ ID NO:430), and c) an adenoviral helper plasmid (adenovirus type 5 (pAd5); see e.g., Vandendriessche et al. (2007) J. Throm. Haem. 5:16-24; Chuah et al., (2014) J. Am. Soc. Gene Ther. 22:1605-1613). After 3 days of incubation, cells were released by the addition of 0.5 mL 500 mM EDTA per flask and crude AAV particle extracts were prepared by freezing and thawing cells for 3-4 times at −80° C. to liberate the virus particles from the HEK-293T cells.

Cells were then digested for 1 hour at 37° C. with 200 U/mL Benzonase® enzyme digestion buffer (EMD Chemicals, Fisher #NC0544951, Waltham, Mass.) to remove non-encapsidated single stranded (ss) and double stranded (ds) DNA and RNA and/or DNA and RNA leaking from broken virus particles. Then, 25 mM CaCl₂) was added to precipitate debris, and supernatants were treated with PEG 8000 (Fisher, M6510, Waltham, Mass.) buffered with 2.5M NaCl to pellet the virus.

All resulting rAAV preparations were processed identically using two rounds of ultrapure optical grade cesium chloride (CsCl, Invitrogen #15507-023, Waltham, Mass.) gradient centrifugation, followed by dialysis using slide-A-Lyzer G2 dialysis cassettes (MWCO 10,000) (Pierce #87730, Waltham, Mass.) according to manufacture instructions for removal of CsCl and particle concentration. Final viral preparations were collected and frozen and stored at −80° C. in phosphate-buffered saline (PBS) containing 5% D-Sorbitol (Sigma #S6021, St. Louis, Mo.).

C. rAAV Titration

Titration of AAV was performed by qPCR using the Real-Time PCR System (Applied Biosystems StepOnePlus, Waltham, Mass.). DNA was extracted using the QIAamp MinElute Virus Spin Kit (Qiagen Cat #57704, Germantown, Md.) according to the manufacturer's instructions. Linearized and purified inverted terminal repeat (ITR) vector containing a sequence in the AAV of known concentration was used as a standard for titration. Serial dilutions of the genomic DNA (gDNA) isolated from purified virus was amplified using forward and reverse primers for Factor IX (Forward: ATCTACAACAACATGTTCTGCG SEQ ID NO: 556 Reverse: CTGATGATGCCGGTCAGAAA SEQ ID NO: 557) using a PCR program set to the following cycle times: 95° C. 10 minute, 40 cycles at 95° C. for 15 seconds and 60° C. for 1 minute. Titer was then assessed by quantitative dot blot and calculated based on a standard curve.

### D. FIX Codon Optimization

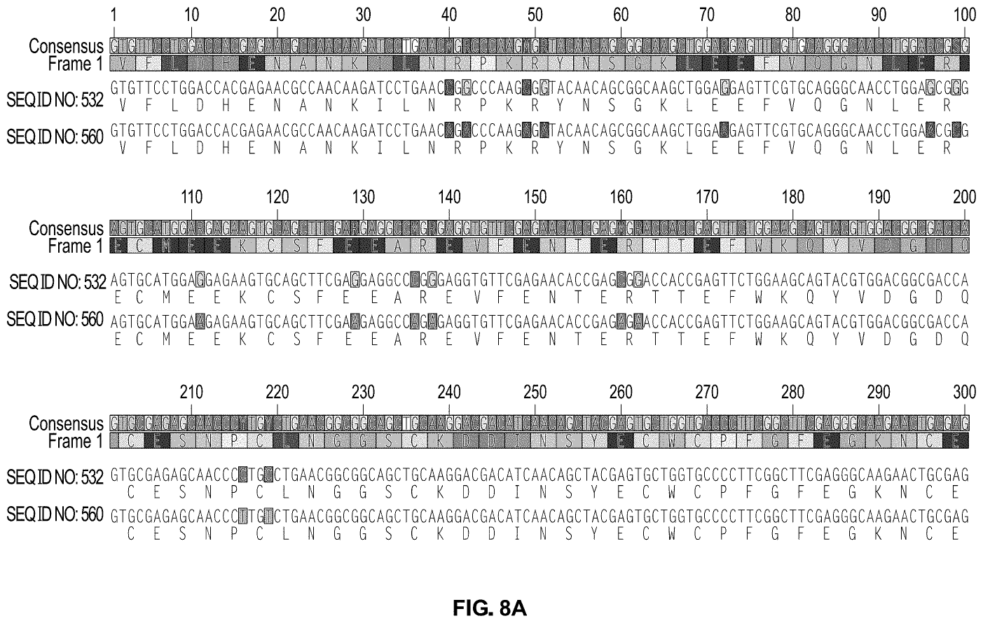

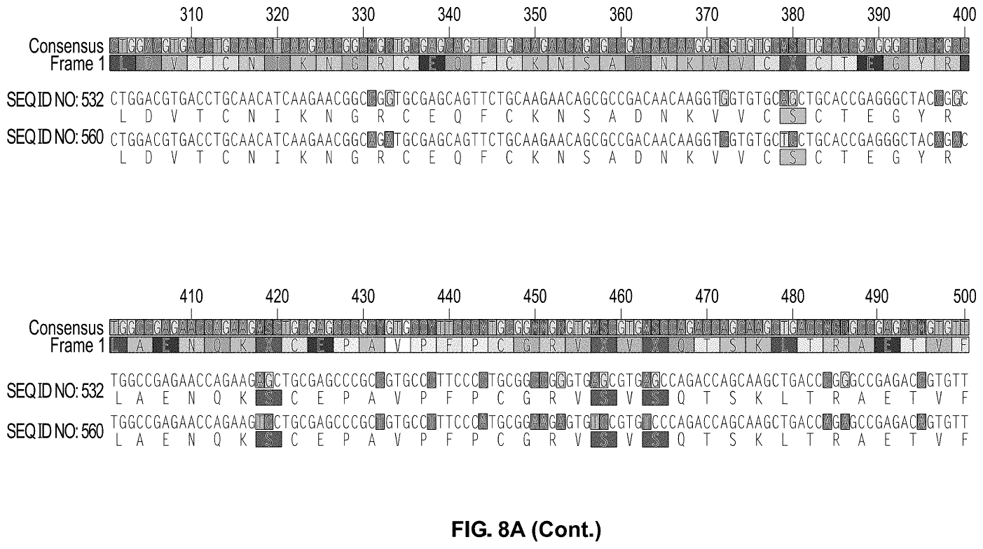

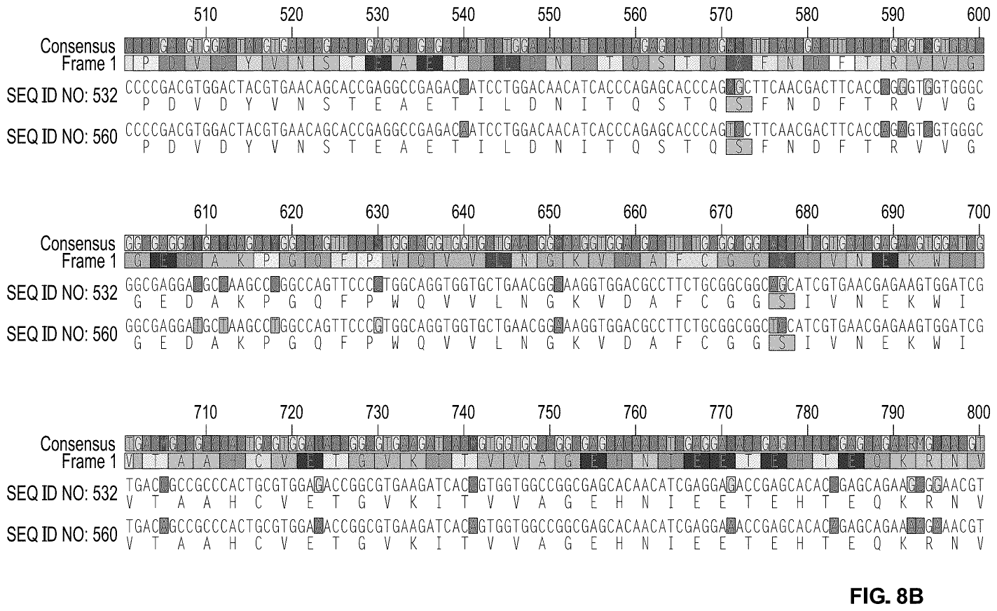

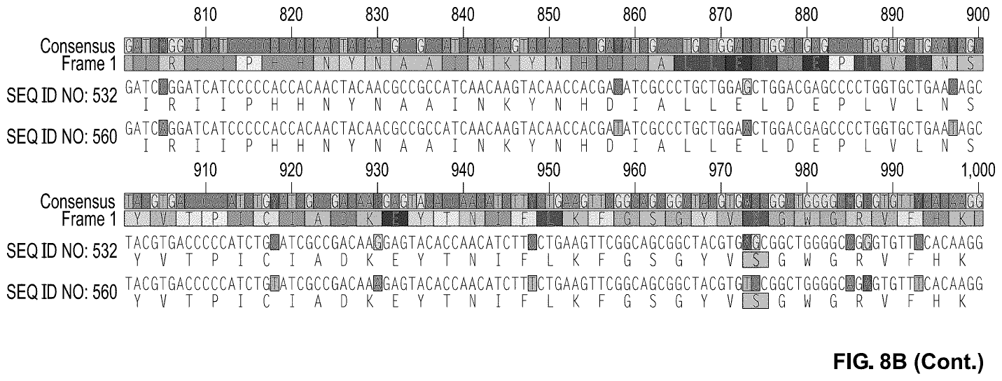

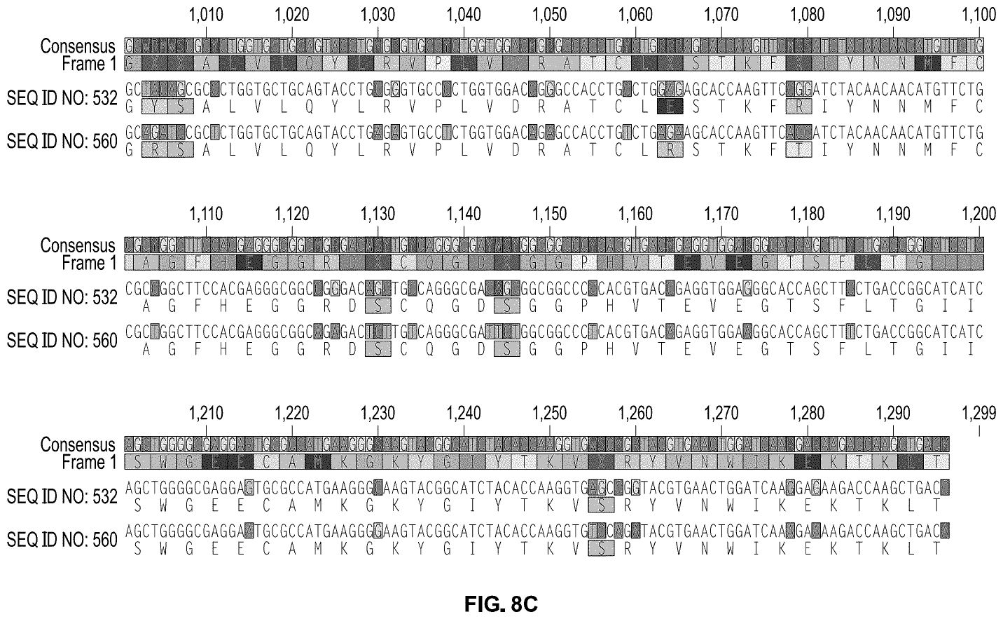

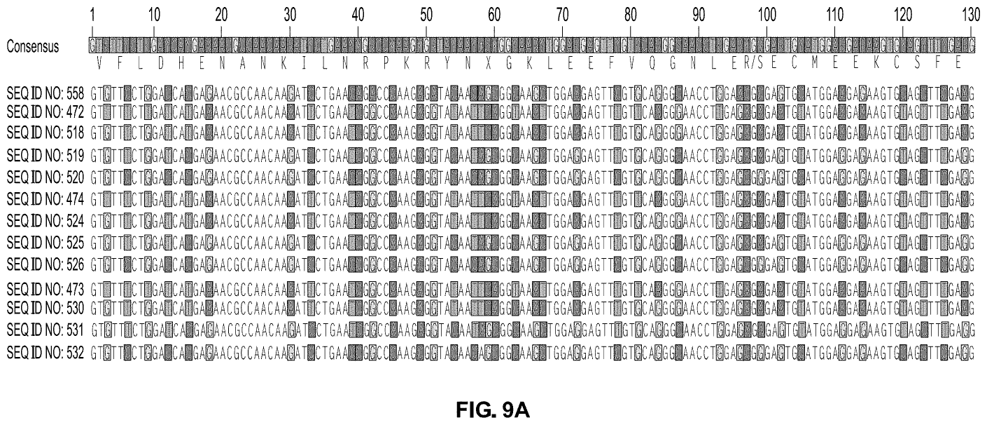

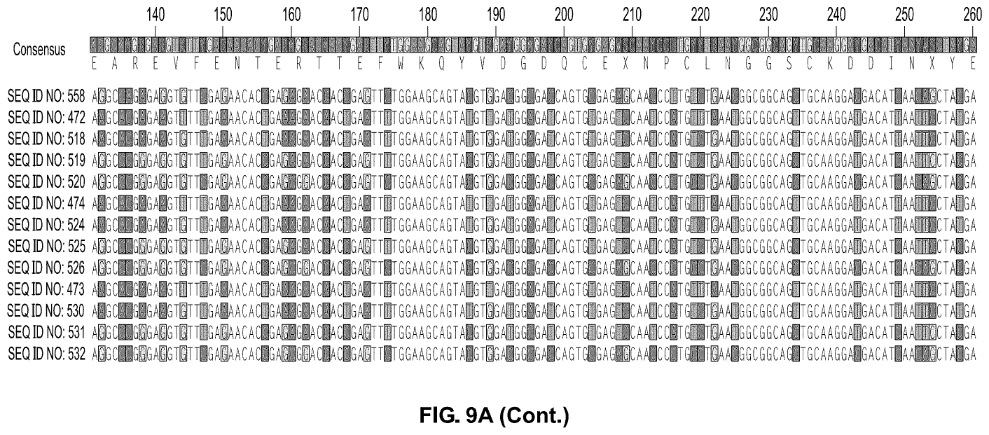

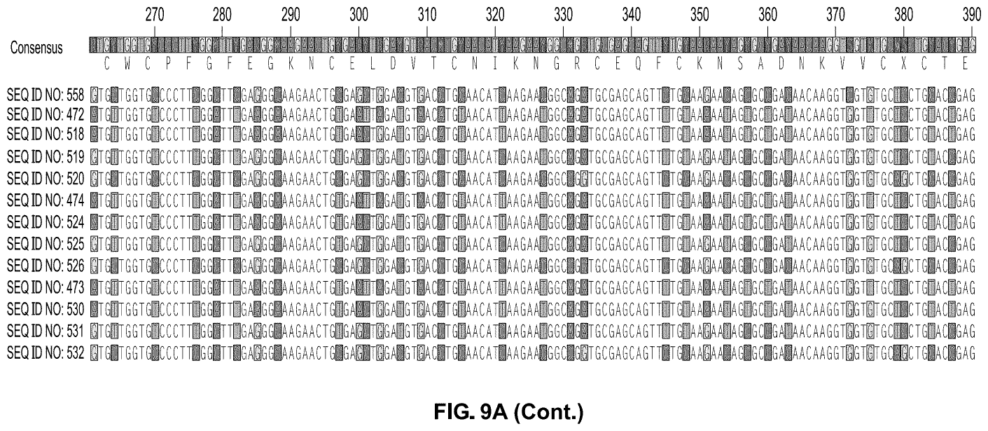

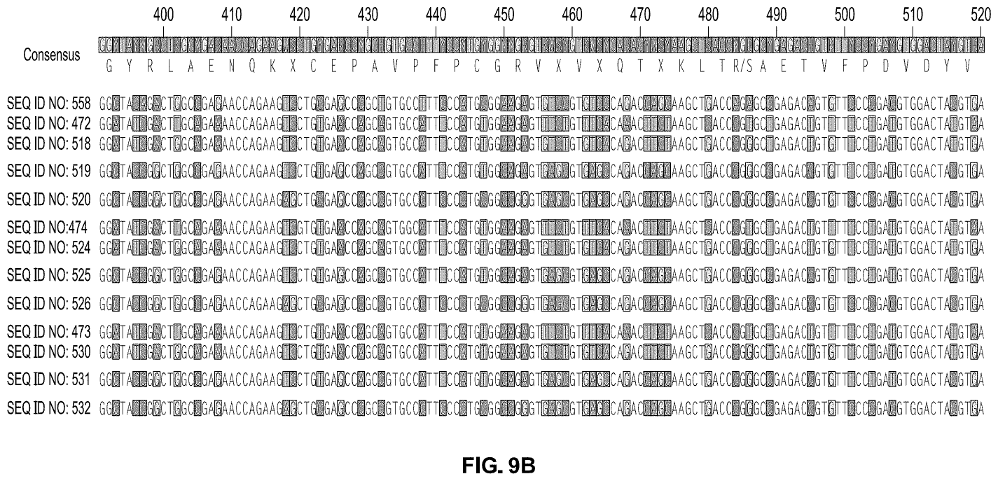

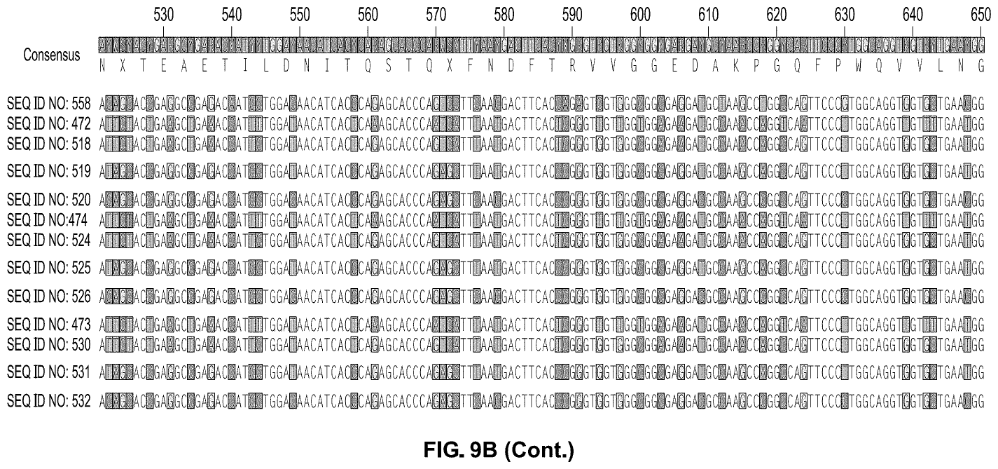

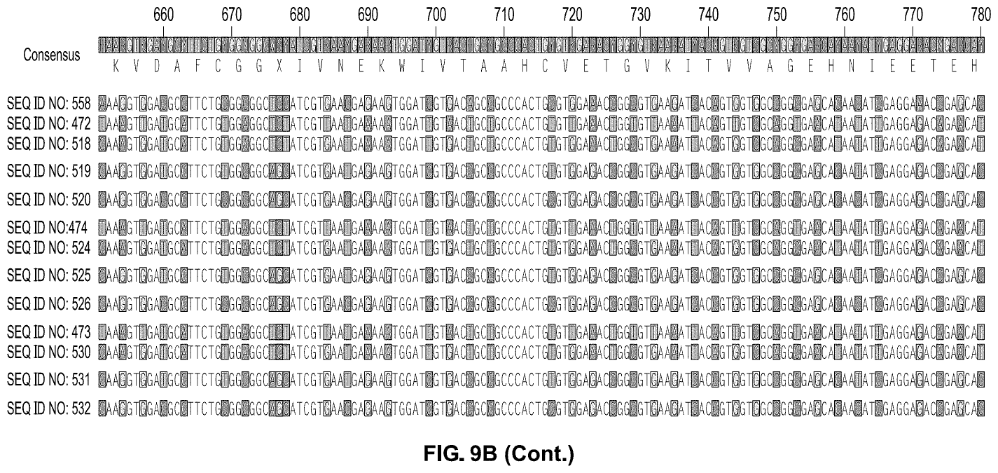

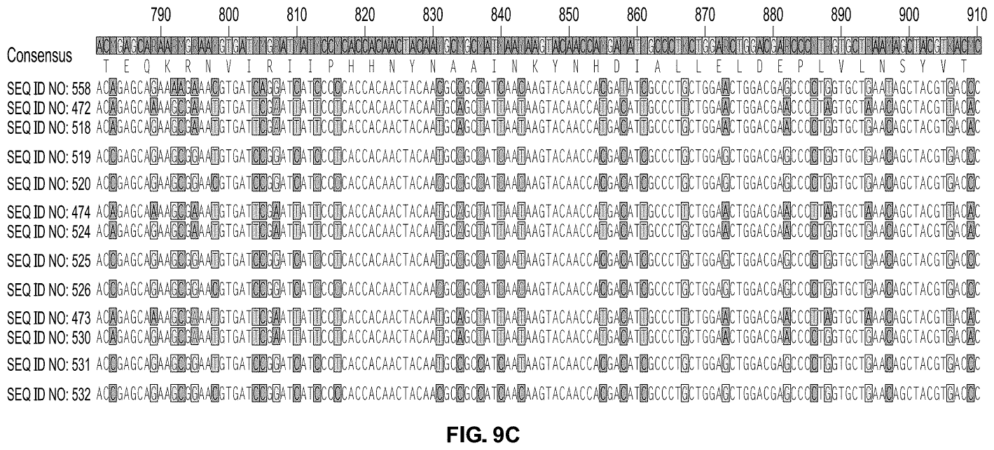

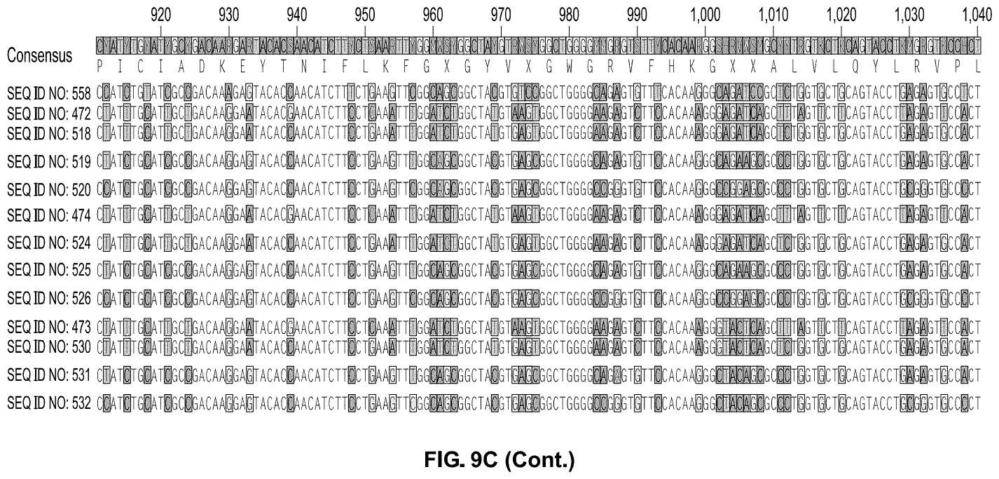

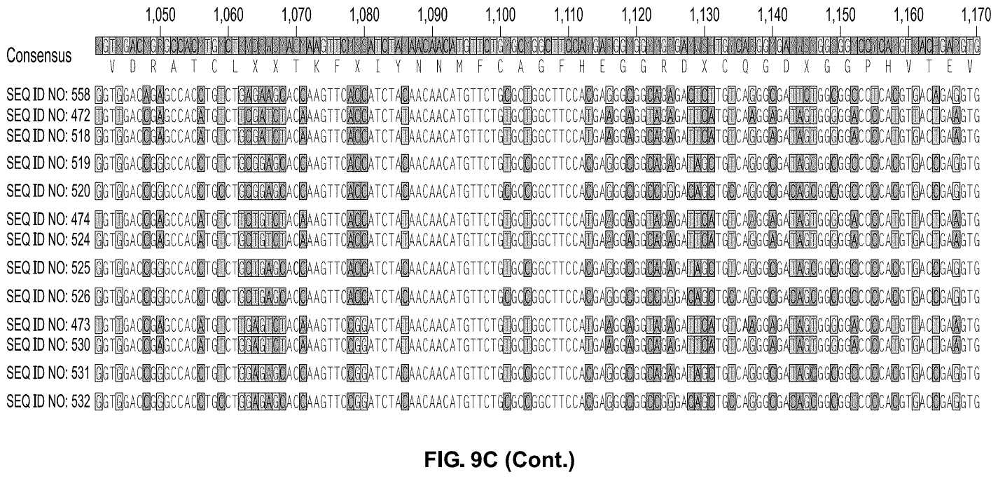

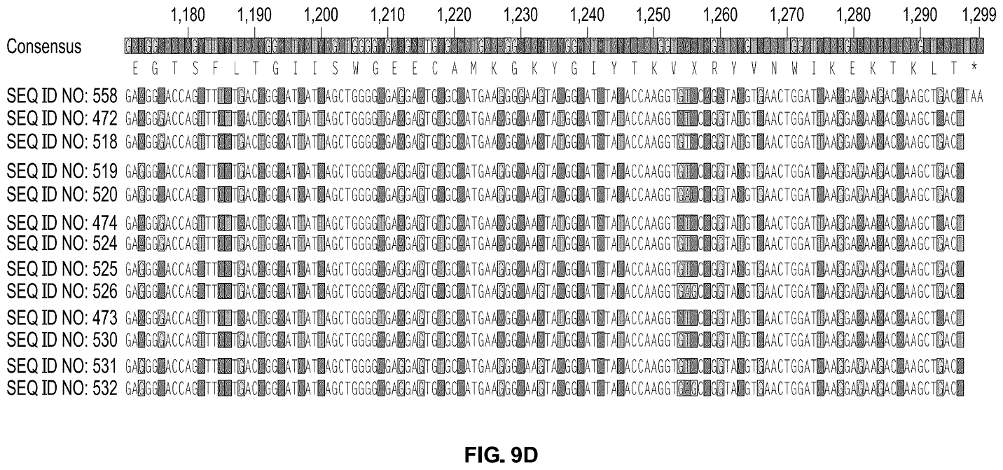

As applicable, FIX DNA sequences were codon optimized for mammalian expression using murine preferences or human preferences using the Geneious Prime software tool. For example, for expressing human wild-type FIX or FIX variants in mouse cell lines or in vivo in mice, the human FIX gene was codon optimized for mouse using the Thermo Scientific GeneArt website tool as previously described (see e.g., Barzel et al. (2015) Nature 517:360-364). In some examples, the human wild-type or FIX variants are codon optimized for mouse and are used for expressing FIX in non-mouse cells, such as human cells or non-human primate cells or animals. In another example, human wild-type FIX or FIX variants for expression in human cells or tissue were codon optimized for human. For example, to codon optimize for human cell line expression, sequences were optimized utilizing ‘standard’ source genetic code, Homo sapiens as the target organism, ‘standard’ target genetic code, and thresholds were set up to be rare (i.e., at thresholds of 0.5, 0.8 and 1.0). The nucleic acid molecules encoding the three FIX polypeptides: wild-type FIX (SEQ ID NO: 3), Padua FIX (SEQ ID NO: 484), and exemplary modified FIX (SEQ ID NO:394), were codon optimized for human expression. As noted above, the sequences with the optimized human codons are very similar (greater than 90% sequence identity). In some examples, the CpG (or CG) islands are removed, such as for optimizing coding sequences for increased expression. In these examples, examples of codon-optimized wild-type FIX, modified FIX (CB2679), and Padua FIX, are set forth in SEQ ID NOs:518-553. Table 43, below, describes the sequences. A sequence alignment comparing wild-type FIX, modified FIX and Padua FIX (frame 1) at thresholds of 0.5, 0.8 and 1.0 is set forth in FIGS. 8A-B and FIGS. 9A-D.

TABLE 43
Nucleic acid sequences encoding FIX and modified FIX
that is codon optimized for expression in humans

| SEQ ID NO. | Portion of FIX | Description |
|---|---|---|
| 518 | encodes 47-461 | human WT-FIX mature codon |
| | | optimized for human 0.5 |
| | | optimization |
| 519 | encodes 47-461 | human WT-FIX-mature codon |
| | | optimized for human 0.8 |
| | | optimization |
| 520 | encodes 47-461 | human WT-FIX-mature codon |
| | | optimized for human 1.0 |
| | | optimization |
| 521 | encodes 47-461 | human FIX R318Y/R338E/T343R |
| | | mature codon optimized for |
| | | human 0.5 optimization |
| 522 | encodes 47-461 | human FIX R318Y/R338E/T343R |
| | | mature codon optimized for |
| | | human 0.8 optimization |
| 523 | encodes 47-461 | human FIX R318Y/R338E/T343R |
| | | mature codon optimized for |
| | | human 1.0 optimization |
| 524 | encodes 47-461 | human FIX R338L mature codon |
| | | optimized for human 0.5 |
| | | optimization |
| 525 | encodes 47-461 | human FIX R338L mature codon |
| | | optimized for human 0.8 |
| | | optimization |
| 526 | encodes 47-461 | human FIX R338L mature codon |
| | | optimized for human 1.0 |
| | | optimization |
| 527 | encodes 30-461 | hWT-FIX codon optimized for |
| | | human 0.5 optimization |

TABLE 43
Nucleic acid sequences encoding FIX and modified FIX
that is codon optimized for expression in humans

| SEQ ID | | |
|---|---|---|
| NO. | Portion of FIX | Description |
| 518 | encodes 47-461 | human WT-FIX mature codon optimized for |
| | | human 0.5 optimization |
| 519 | encodes 47-461 | human WT-FIX-mature codon optimized for |
| | | human 0.8 optimization |
| 520 | encodes 47-461 | human WT-FIX-mature codon optimized for |
| | | human 1.0 optimization |
| 521 | encodes 47-461 | human FIX R318Y/R338E/T343R mature codon |
| | | optimized for human 0.5 optimization |
| 522 | encodes 47-461 | human FIX R318Y/R338E/T343R mature codon |
| | | optimized for human 0.8 optimization |
| 523 | encodes 47-461 | human FIX R318Y/R338E/T343R mature codon |
| | | optimized for human 1.0 optimization |
| 524 | encodes 47-461 | human FIX R338L mature codon optimized for |
| | | human 0.5 optimization |
| 525 | encodes 47-461 | human FIX R338L mature codon optimized for |
| | | human 0.8 optimization |
| 526 | encodes 47-461 | human FIX R338L mature codon optimized for |
| | | human 1.0 optimization |
| 527 | encodes 30-461 | hWT-FIX codon optimized for human 0.5 |
| | | optimization |
| 528 | encodes 30-461 | hWT-FIX codon optimized for human 0.8 |
| | | optimization |
| 529 | encodes 30-461 | hWT-FIX codon optimized for human 1.0 |
| | | optimization |
| 530 | encodes 30-461 | human FIX R318Y/R338E/T343R codon |
| | | optimized for human 0.5 optimization |
| 531 | encodes 30-461 | human FIX R318Y/R338E/T343R codon |
| | | optimized for human 0.8 optimization |
| 532 | encodes 30-461 | human FIX R318Y/R338E/T343R codon |
| | | optimized for human 1.0 optimization |
| 533 | encodes 30-461 | human FIX R338L codon optimized for human |
| | | 0.5 optimization |
| 534 | encodes 30-461 | human FIX R338L codon optimized for human |
| | | 0.8 optimization |
| 535 | encodes 30-461 | human FIX R338L codon optimized for human |
| | | 1.0 optimization |
| 536 | precursor + intron | hFIXss-Pro-mini Intron-Pro-human wild type |
| | | FIX optimized 0.5 |
| 537 | precursor + intron | hFIXss-Pro-mini Intron-Pro-human wild type |
| | | FIX optimized 0.8 |
| 538 | precursor + intron | hFIXss-Pro-mini Intron-Pro-human wild type |
| | | FIX optimized 1.0 |
| 539 | precursor + intron | hFIXss-Pro-mini Intron-Pro-human FIX |
| | | R318Y/R338E/T343R optimized 0.5 |
| 540 | precursor + intron | hFIXss-Pro-mini Intron-Pro-human FIX |
| | | R318Y/R338E/T343R optimized 0.8 |
| 541 | precursor + intron | hFIXss-Pro-mini Intron-Pro-human FIX |
| | | R318Y/R338E/T343R optimized 1.0 |
| 542 | precursor + intron | hFIXss-Pro-mini Intron-Pro-human FIX |
| | | R338L optimized 0.5 |
| 543 | precursor + intron | hFIXss-Pro-mini Intron-Pro-human FIX |
| | | R338L optimized 0.8 |
| 544 | precursor + intron | hFIXss-Pro-mini Intron-Pro-human FIX |
| | | R338L optimized 1.0 |
| 545 | precursor + intron | mouse optimized hFIXss-Pro-mini Intron-Pro- |
| | | human wild type FIX optimized 0.5 |
| 546 | precursor + intron | mouse optimized hFIXss-Pro-mini Intron-Pro- |
| | | human wild type FIX optimized 0.8 |
| 547 | precursor + intron | mouse optimized hFIXss-Pro-mini Intron-Pro- |
| | | human wild type FIX optimized 1.0 |
| 548 | precursor + intron | mouse optimized hFIXss-Pro-mini Intron-Pro- |
| | | human FIX R318Y/R338E/T343R optimized 0.5 |
| 549 | precursor + intron | mouse optimized hFIXss-Pro-mini Intron-Pro- |
| | | human FIX R318Y/R338E/T343R optimized 0.8 |
| 550 | precursor + intron | mouse optimized hFIXss-Pro-mini Intron-Pro- |
| | | human FIX R318Y/R338E/T343R optimized 1.0 |
| 551 | precursor + intron | mouse optimized hFIXss-Pro-mini Intron-Pro- |
| | | human FIX R338L optimized 0.5 |
| 552 | precursor + intron | mouse optimized hFIXss-Pro-mini Intron-Pro- |
| | | human FIX R338L optimized 0.8 |
| 553 | precursor + intron | mouse optimized hFIXss-Pro-mini Intron-Pro- |
| | | human FIX R338L optimized 1.0 |

### Example 12

### FIX Production in Huh-7 Cells

rAAV modified FIX (CB2679), Padua FIX and wild-type FIX plasmids, described above in Example 10 and set forth in SEQ ID NOs:447-449 with the FIX codon optimized for expression in mouse (SEQ ID NOs:558-560), were transfected into the human hepatocellular carcinoma cell line Huh-7 and protein expression was assessed. Since the experiments are conducted in a mouse model, the codons were optimized for expression in mouse. The FIX sequences of the mouse optimized codons (SEQ ID NOs:558-560) share more than 90% sequence identity to the highest stringency human codon optimized sequences (SEQ ID NOs: 529, 532, 535). As a control a parental mini circle plasmid containing the same construct with the native, i.e. non-codon optimized, huFIX sequence was transfected in parallel.

4.5×10⁵ cells were seeded in a volume of 3 mL per well of a 6-well plate. The next morning, the cells were transfected with 2.5 μg of each construct using Lipofectamine 3000 according to the manufacturer's instructions. 3 days post transfection, cell supernatants were collected. Next, cells were rinsed with DPBS, and lysed in 350 μL mammalian protein extraction reagent (M-PER®; Thermo Fisher Scientific) and cell lysates were harvested. Cell debris was removed by centrifugation and cell lysates and supernatants were analyzed for huFIX antigen expression by ELISA and Western Blot (WB). The results show that codon optimized huFIX, which share more than 90% sequence identity with the mouse optimized FIX, constructs expressed at higher levels than the non-codon optimized constructs. The results are set forth in Table C1, below.

Next, hFIX expression in Huh-7 cells transfected with the vectors was assessed. rAAV was prepared on a small scale using the AAVpro® purification kit (Takara, Cat No. 6666) rAAV vector containing wild-type hFIX packaged with a chimeric capsid (SEQ ID NO: 418) also was transduced, at a multiplicity of infection (MOI) of 20,000 vector genomes (vg) per cell. Cell lysates and supernatants were harvested 3 days post transduction and huFIX expression was assessed by ELISA and western blot.

The results are set forth in Table 44, below. The results show that the rAAV construct packaged with the capsid designated KP1 is capable of transducing Huh-7 cells with high efficiency. In cells transfected with the plasmids alone, codon optimized FIX expressed at higher levels than non-codon optimized FIX. Wild type FIX, Padua FIX and modified FIX all expressed at similar levels in Huh-7 cells.

TABLE 44
huFIX antigen levels after transfection of Huh-7 cells with
FIX constructs or transduction with rAAV expressing FIX.

| | | huFIX in | huFIX in Cell |
|---|---|---|---|
| | | Supernatants | Lysates |
| Delivery | Sample | (ng/mL) | (ng/mL) |
| Transfection | WT FIX | 106.3 | 93.4 |
| (plasmid) | | | |
| Transfection | Padua FIX | 94.7 | 70.7 |
| (plasmid) | | | |
| Transfection | Modified FIX | 83.6 | 59.5 |
| (plasmid) | (SEQ ID NO: 394) | | |
| Transfection | WT FIX, non-codon opt. | 27.0 | 20.1 |
| (plasmid) | | | |
| Transduction | WT FIX + Capsid | 219.3 | 166.1 |
| (rAAV) | set forth in | | |
| | SEQ ID NO: 418 (KP1) | | |

### Example 13

### FIX Expression in a Mouse Hemophilia Model Treated with the AAV Vector Designated DJ8

FIX protein expression and activity in mouse plasma after injection of a recombinant adeno-associated viral vector (rAAV) encoding and expressing modified FIX (mature form set forth in SEQ ID NO:394, except that residue 148 is A; SEQ ID NO:486) packaged in capsid designated DJ8 (SEQ ID NO:427) for comparison with the rAAV using the vectors and constructs provided herein (see, Example 14 below, in which constructs prepared as described herein are packaged in capsids provided herein.

In vivo transduction efficiency of rAAV packaged with the previously characterized AAV packaging construct encoding AAV/DJ8 (Bio-connect, The Netherlands; Grimm et al., J. Virol. 82:5887-5911 (2008); SEQ ID NO: 427) and encoding and expressing modified FIX (mature sequence set forth in SEQ ID NO:394, except for this example the residue at position 148 is A (alanine), sequence set forth in SEQ ID NO:486) was examined in mice. C57BL/6 FIX-deficient mice were injected with rAAV FIX vector packaged with the DJ8 capsid, and FIX expression and coagulation activity was monitored over several weeks.

### A. Cloning of the FIX Gene

Codon-optimized (co) human (h) FIX-R338L-Padua cDNA (Padua) or co-hFIX-CB 2679d-GT (modified FIX R318Y/R338E/T343R, mature sequence set forth in SEQ ID NO:394 where the amino acid at position 148 is A) cloned downstream of a liver-specific promoter (α1-anti-trypsin promoter, AAT) was cloned into a self-complementary (sc) AAV backbone (scAAV) (see e.g., McCarty et al., (2003) Gene Therapy 10:2112-2118) containing a mini-intron from minute virus of mice (MVM) upstream of the co-hFIX transgene and a bovine growth hormone polyadenylation signal (bGHpA). The Padua and modified FIX constructs contain the T148A residue; a known polymorphism in FIX. Both plasmids were verified by restriction digestion and Sanger sequencing using 13 different primers spanning the entire expression cassette and the scAAV backbone. The final AAV vector contains the AAT promoter-MVM-co-hFIX (Padua or CB2679, where the amino acid at position 148 is A)-bGHpA.

B. rAAV Vector Production

The unmodified FIX employed for these experiments was the allele that encodes T148A. rAAV vectors for producing the modified FIX (mature SEQ ID NO:486, which is SEQ ID NO:394 with the replacement T148A) or Padua FIX (SEQ ID NO:491) packaged with the DJ/8 capsid (SEQ ID NO:427) were generated using triple plasmid calcium phosphate transfection (Invitrogen Corp, Carlsbad, Calif., USA) of HEK-293T/17 cells with (1) the AAV plasmid of interest (pAAV-AAT-hFIX), (2) a chimeric AAV packaging construct encoding AAV/DJ8, and (3) an adenoviral helper plasmid. All plasmids required for AAV production were extracted using an endotoxin free maxiprep protocol (Thermo Fischer Scientific, Belgium). The AAV vectors were produced at high titer (5.2×10¹²-1.3×10¹³ vg/mL). rAAV was purified from the cell lysates by three rounds of cesium chloride (CsCl) gradient ultracentrifugation purification (see e.g., Grimm et al, J. of Virology 80(1):426-39 (2006)).

HEK-293T (ATCC #CRL-3216, Manassas, Va.) were seeded and expanded for 2 days prior to transfection in T225 flasks (Corning Inc., Corning, N.Y.). The cells were then transfected using a standard calcium phosphate-based protocol with a) hFIX-encoding rAAV vector plasmid (modified FIX or Padua), b) AAV serotype-specific packaging plasmid (AAV/DJ8), and c) adenoviral helper plasmid (adenovirus type 5 (pAd5); see e.g., Vandendriessche et al., J. Throm. Haem. (2007) 5:16-24; Chuah et al., J. Am. Soc. Gene Ther. (2014) 22:1605-1613). Two days post transfection, cells were harvested. Harvested cells were lysed by successive freeze/thaw cycles and sonication, digested for 1 hour at 37° C. with 200 U/mL Benzonase® digestion buffer (EMD Chemicals, Fisher #NC0544951, Waltham, Mass.) and deoxycholic acid (Sigma-Aldrich, St Louis, Mo., USA) to remove non-encapsidated single stranded (ss) and double stranded (ds) DNA and RNA and/or DNA and RNA leaking from broken virus particles.

All resulting rAAV preparations were processed identically using three rounds of ultrapure optical grade cesium chloride (CsCl, Invitrogen #15507-023, Waltham, Mass.) gradient centrifugation. Fractions containing the AAV vector were collected, concentrated and dialyzed into 1 mM MgCl₂ in Dulbecco's phosphate buffered saline (PBS; Gibco, BRL).

Vector titers (in viral genomes (vg) per mL) were determined by quantitative real-time polymerase chain reaction (qPCR) using vector-specific primer pairs. For all vectors, primers specific for the bGHpA sequence were used. The forward and reverse primers used were 5′-GCCTTCTAGTTGCCAGCCAT-3′ and 5′-GGCACCTTCCAGGGTCAAG-3′(SEQ ID NOs: 512 and 513, respectively). Reactions were performed with SYBR® Green PCR Master Mix, according to the manufacturer's instructions on an ABI 7500 Real-Time PCR System. Known copy numbers (102-108) of the respective vector plasmids were used to generate the standard curves. The AAV vectors were produced at high titer (5.2×10¹²-1.3×10¹³ vg/mL).

C. FIX Protein Expression in Plasma after Intravenous Injection of rAAV-Modified FIX and Padua FIX in a Mouse Model of Hemophilia B

C57BL/6 F9-deficient mice (3-5 mice/group) were injected via the tail vein with either 4 E+10, 2 E+11 or 4 E+11 vector genomes per kg (vg/kg) of rAAV FIX vector for expressing either modified FIX or Padua FIX assuming a mean animal weigh t of 25 grams. Whole blood was collected by phlebotomy of the retro orbital plexus at 1, 3, 5, 7, 8, 9, 12, 16 and 20 weeks post-intravenous dosing and plasma was prepared for bioanalytical assays.

Blood collected from the retro orbital plexus was diluted in 20% sodium citrate butter to a final amount of 0.4%. Plasma was prepared by centrifugation at 13,000 RPM at 4° C. for three minutes and treated blood samples were placed on dry ice and stored at −80° C. Blood and plasma samples were kept on ice throughout collection and processing. Plasma from non-injected or vehicle-injected mice were used as negative controls.

FIX concentrations in plasma were determined using a FIX enzyme-linked immunosorbent antigen assay (ELISA) specific for hFIX antigen (ASSERACHROM IX: Ag Enzyme Immunoassay for Factor FIX; Diagnostica Stago, France) according to the manufacturer's instructions using known concentrations of recombinantly expressed modified FIX or Padua FIX as controls.

The results show that modified FIX and Padua FIX expressed at similar levels; expression was maintained for at least 20 weeks post vector-injection in a dose-dependent manner.

D. FIX Activity in Plasma after Intravenous Injection of rAAV-Modified FIX and Padua FIX in a Mouse Model of Hemophilia B

rAAV FIX vectors expressing the modified FIX (CB2679) and Padua FIX (FIX R338L) were produced as detailed above in Sections A and B. C57BL/6 FIX-deficient mice (3-5 mice/group) were injected via the tail vein with either 4 E+10, 2 E+11 or 4 E+11 vector genomes per kg (vg/kg) of rAAV FIX vector expressing the modified FIX (or Padua FIX. Blood was collected retro-orbitally at weeks 1, 3, 5, 7, 8, 9, 12, 16 and 20 post-intravenous dosing, and plasma was prepared for examination of the functional coagulation activity of the modified FIX and Padua FIX. The results are set forth in Tables 45-47, below.

FIX activity levels were determined using an activated partial thromboplastin time (aPTT)-based Factor IX single-stage clotting assay. The aPTT-assay was performed on an ACL-TOP instrument (Instrumentation Laboratories (Bedford, Mass.)) using indicated reagents and protocols. Briefly, an aPTT reagent, sold as HemosIL® or SynthasIL was equilibrated at 37° C. and pre-incubated with dilutions of mouse plasma harvested at various time points from mice treated with the rAAV, according to the manufacturer's instructions. Clotting was induced through the automated addition of 20 mM calcium chloride, which triggers the coagulation process. For FIX clotting times, activity was also assessed using an aPTT-based Factor IX single-stage clotting assay, however, a different reagent set was employed as per the manufacturer's instructions (C.K. PREST kit and Start Max; Stago).

The results show that FIX activity, as determined by the aPTT-assay, was restored with modified FIX and Padua FIX expression. The modified FIX (SEQ ID NO:490) showed a significant increase in activity compared to vehicle control at all dose levels with the highest vector dose showing stable FIX activity level of 5-6 U/mL. Similarly, clotting times were reduced after administration of 4 E+10, 2 E+11 or 4 E+11 vg/kg of rAAV FIX vectors, compared to vehicle control (see Tables 45 and 46, below).

Phenotypic efficacy was assessed using a standard mouse tail-clip assay and following the blood loss and the bleeding time. Briefly, the mice were anesthetized, and the tails were placed in a pre-warmed saline solution for two minutes and subsequently cut at a 2.5 mm diameter. The tails were then immediately returned to the 37° C. saline solution and the bleeding time monitored until no further bleeding occurred or the end of study. Blood-containing saline was centrifuged at 520 g for 10 minutes at 4° C. to collect erythrocytes, which were then resuspended in 6 mL of lysis buffer (10 mM KHCO₃, 150 mM NH₄Cl, 0.1 mM EDTA). Lysis proceeded for 10 minutes at room temperature and samples were centrifuged again, at 520 g for 10 minutes at 4° C. The absorbance of the supernatants was measured at 570 nm spectroscopically to determine the amount of hemoglobin as an indication of blood loss. The bleeding time was significantly reduced (p<0.001) for all doses of modified FIX and Padua FIX compared to vehicle control. Blood loss volume also was reduced significantly in mice administered both rAAV FIX vectors (p<0.001), compared to vehicle control. Of note, the bleeding time was reduced 4-5-fold for mice receiving 2 E+11 and 4 E+11 vg/kg of modified FIX compared to Padua FIX (p<0.01) (see Table 47).

The increased FIX activity in the one-stage clotting assays and significantly reduced blood loss following gene therapy was similar in mice administered AAV-modified FIX and AAV-Padua (FIX R338L), and both FIX proteins showed significantly increased FIX expression and activity compared to vehicle alone. AAV-modified FIX (CB2679) demonstrated superior reduction in blood loss compared to AAV-FIX R338L.

TABLE 45
Modified FIX and Padua FIX activity in plasma after 4E+10, 2E+11 or 4E+11 vector genomes per
kg (vg/kg) administered intravenously via tail vein (values are ± standard deviation; nd = not determined).

| | | FIX activity in U/mL | | FIX activity in U/mL | | FIX activity in U/mL | |
|---|---|---|---|---|---|---|---|
| | | (4E+11 vg/kg) | | (2E+11 vg/kg) | | (4E+10 vg/kg) | |
| | Vehicle | modified | FIX- | Modified | FIX- | Modified | FIX- |
| week | PBS | FIX* | R338L | FIX | R338L | FIX | R338L |
| 1 | 0.11 ± 0.03 | 6.62 ± 1.92 | 4.48 ± 1.07 | 4.87 ± 1.40 | 2.39 ± 0.58 | nd | nd |
| 3 | nd | nd | nd | nd | nd | 0.92 ± 0.13 | 0.56 ± 0.16 |
| 5 | 0.12 | nd | nd | nd | nd | 0.90 ± 0.16 | 0.6 ± 0.17 |
| 7 | nd | 6.36 ± 0.97 | 5.68 ± 1.28 | 4.29 ± 0.60 | 2.79 ± 0.89 | nd | nd |
| 8 | nd | nd | nd | nd | nd | 0.79 ± 0.19 | 0.47 ± 0.147 |
| 9 | nd | 6.48 ± 1.20 | 5.54 ± 1.23 | 4.18 ± 1.35 | 2.81 ± 0.89 | nd | nd |
| 12 | 0.13 ± 0.04 | 5.32 ± 1.37 | 5.12 ± 1.49 | 4.02 ± 1.11 | 2.74 ± 0.93 | 0.97 ± 0.25 | 0.73 ± 0.16 |
| 16 | 0.20 ± 0.01 | 5.78 ± 1.61 | 4.96 ± 1.35 | 4.13 ± 0.89 | 2.63 ± 0.94 | 0.89 ± 0.29 | 0.70 ± 0.15 |
| 20 | 0.17 ± 0.04 | 5.66 ± 1.34 | 4.94 ± 1.00 | 4.38 ± 1.00 | 3.15 ± 0.98 | nd | nd |

*modified FIX is the FIX contain R318Y/R338E/T343R replacements (SEQ ID NO: 394 except with the T128A allele)

TABLE 46
Clotting time (in seconds) after 4E+10, 2E+11 or 4E+11 vector genomes per kg (vg/kg)
modified FIX (SEQ ID NO: 490) or Padua FIX (SEQ ID NO: 491) administered intravenously via tail vein.

| | Clotting time (seconds) | | | Clotting time (seconds) | | | Clotting time (seconds) | | |
|---|---|---|---|---|---|---|---|---|---|
| | (4E+11 vg/kg) | | | (2E+11 vg/kg) | | | (4E+10 vg/kg) | | |
| Week | FIX* | Padua | Vehicle | FIX* | Padua | Vehicle | FIX* | Padua | Vehicle |
| 1 | 16.1 ± 2.6 | 22.0 ± 1.5 | 49.7 ± 3.1 | 15.1 ± 2.5 | 21.4 ± 2.1 | 49.7 ± 3.1 | 23.0 ± 1.6 | 27.1 ± 3.3 | 50.0 ± 3.0 |
| 3 | 18.1 ± 1.7 | 18.5 ± 1.1 | 44.9 ± 6.8 | 17.4 ± 2.4 | 22.7 ± 3.5 | 44.9 ± 6.8 | 22.8 ± 1.6 | 25.7 ± 2.1 | 56.9 ± 4.1 |
| 5 | 15.5 ± 3.1 | 23.2 ± 1.2 | 50.8 ± 3.8 | 16.6 ± 2.4 | 25.1 ± 2.3 | 50.8 ± 3.8 | 25.7 ± 1.2 | 28.7 ± 0.5 | 53.4 ± 4.4 |
| 7 | 15.8V1.2 | 21.3 ± 1.7 | 56.9 ± 4.1 | 16.9 ± 1.7 | 24.5 ± 2.3 | 56.9 ± 4.1 | nd | nd | nd |
| 8 | nd | nd | nd | nd | nd | nd | 27.7 ± 0.5 | 30.3 ± 0.8 | 57.1 ± 5.9 |
| 9 | 15.9 ± 0.6 | 23.0 ± 2.6 | 53.4 ± 4.4 | 16.5 ± 1.6 | 26.3 ± 2.4 | 53.4 ± 4.4 | nd | nd | nd |
| 12 | 17.7 ± 3.1 | 21.5 ± 2.7 | 57.1 ± 5.9 | 17.1 ± 1.8 | 26.0 ± 2.1 | 57.1 ± 5.9 | 30.6 ± 3.1 | 38.4 ± 3.0 | 62.3 ± 5.1 |
| 16 | 22.8 ± 2.8 | 27.1 ± 2.8 | 62.3 ± 5.1 | 23.0 ± 3.2 | 29.5 ± 1.2 | 62.3 ± 5.1 | 31.9 ± 1.7 | 35.4 ± 2.7 | 60.1 ± 5.0 |
| 20 | 21.9 ± 2.1 | 28.7 ± 2.8 | 60.1 ± 5.0 | 22.5 ± 3.4 | 29.9 ± 2.2 | 60.1 ± 5.0 | nd | nd | nd |

FIX* is the modified FIX whose mature sequence is set forth in SEQ ID NO: 394, except with the T148A allele (see SEQ ID NO: 490)

TABLE 47
Blood loss (Hb at 570 nm) and bleeding time (in minutes) after 4E+10
or 2E+11 vector genomes per kg modified FIX (SEQ ID NO: 490)
or Padua FIX (SEQ ID NO: 491) administered intravenously via tail
vein (values are ± standard deviation; nd = not determined).

| | Blood Loss (Hb) | | | Blood Loss (Hb) | | |
|---|---|---|---|---|---|---|
| | (4E+10 vg/kg) | | | (2E+11 vg/kg) | | |
| Week | FIX* | Padua | Vehicle | FIX* | Padua | Vehicle |
| 20 | 0.26 ± 0.10 | 0.13 ± 0.07 | 1.18 ± 0.33 | 0.15 ± 0.03 | 0.19 ± 0.04 | 1.33 ± 0.32 |
| | Bleeding time (minutes) | | | Bleeding time (minutes) | | |
| | (4E+10 vg/kg) | | | (2E+11 vg/kg) | | |
| Week | FIX* | Padua | Vehicle | FIX* | Padua | Vehicle |
| 20 | 7.4 ± 4.6 | 29.4 ± 12.1 | 63.3 ± 4.8 | 5.2 ± 2.4 | 24.6 ± 9.2 | 63.3 ± 4.8 |

FIX* is the modified FIX whose mature sequence is set forth in SEQ ID NO: 394

E. FIX Transduction Efficiency and Biodistribution after Intravenous Injection of rAAV-Modified FIX and rAAV-Padua FIX in a Mouse Model of Hemophilia B

Quantitative PCR assessments were performed to determine vector copy number per cell and RNA expression in a panel of 8 organs from mice injected with 2 E+11 or 4 E+11 vg/kg of AAV-modified FIX (SEQ ID NO:486 or 394 with the replacement T148A) or AAV-Padua FIX. RNA expression of the transgene was normalized to mouse GAPDH expression.

Mice were euthanized and a panel of organs was collected for further DNA and RNA analyses. Genomic DNA and RNA was extracted from different tissues using the AllPrep DNA/RNA Mini Kit (Qiagen, Chatsworth, Calif., USA) according to the manufacturer's instructions. 100-150 ng of genomic DNA was analyzed using qPCR on an ABI Prism 7900HT (Applied Biosystems, Foster City/CA, USA) and GoTaq® qPCR Master Mix (Promega, Madison, Wis., USA) with bghpA specific forward (5′-GCCTTCTAGTTGCCAGCCAT-3′) and reverse (5′-GGCACCTTCCAGGGTCAAG-3′) primers, according to the manufacturer's instructions.

To generate standard curves, known copy numbers of the corresponding vector plasmid were used. The isolated RNA was used for cDNA synthesis. 100 ng of RNA was reverse transcribed into cDNA by the GoScript™ Reverse Transcription system (Promega, Madison, Wis., USA) and analyzed by qPCR (ABI Prism 7900HT, Applied Biosystems, Foster City/CA, USA) and GoTaq® qPCR Master Mix (Promega, Madison, Wis., USA) with bghpA specific forward (5′-GCCTTCTAGTTGCCAGCCAT-3′) and reverse (5′-GGCACCTTCCAGGGTCAAG-3′) primers (SEQ ID NOs: 512 and 513 respectively). Expression levels were normalized to murine glyceraldehyde-3-phosphate dehydrogenase (GAPDH) mRNA expression, obtained by using the forward primer 5′-TGTGTCCGTCGTGGATCTGA-3′ and reverse primer 5′-GCCTGCTTCACCACCTTCTTGA-3′ (SEQ ID NOs: 516 and 517 respectively). Expression levels were determined based on the 2-ΔcT method.

The results show that the liver was predominately transduced with both vectors and to a lesser extent other organs were transduced, consistent with the known biodistribution of the AAV/DJ-8 serotype (see e.g., Grimm et al., J. Virol. 82:5887-9141 (2008)). Similarly, mRNA expression analysis by quantitative real-time PCR showed that modified FIX (SEQ ID NO: 394) and Padua FIX (SEQ ID NO: 488) were exclusively restricted to the liver by virtue of the liver-specific AAT promoter (see, e.g., Grimm et al., J. Virol. 82:5887-9141 (2008)).

F. Immune Response and Toxicity after Intravenous Injection of Modified FIX and Padua FIX in a Mouse Model of Hemophilia B

To assess the immune consequences of expressing modified FIX and Padua FIX protein at high levels following AAV-based gene therapy, the anti-FIX antibody response was examined.

Anti-FIX antibody titers were analyzed using a modified ELISA protocol. Briefly, 96-well microtiter plates were coated with purified recombinant FIX (SEQ ID NO: 394) or Padua FIX (SEQ ID NO:488) at 1 μg/mL. Serially diluted reference standards were prepared with purified mouse IgG (Invitrogen, Europe). As a positive control, mice were injected with a FIX vector expressing wild-type FIX from the ubiquitously expressed cytomegalovirus (CMV) promoter which invariably results in high-titer anti-FIX antibodies.

The plates were incubated overnight at 4° C. On day two, the samples of mouse plasma taken at various time points during the study were diluted in dilution buffer, loaded on the pre-coated plates and incubated for two hours at room temperature. Experimental plasma samples were obtained from mice injected with different doses of the AAV vector. The plates were then incubated with horseradish peroxidase (HRP) conjugated goat anti-mouse IgG (Invitrogen, Europe) as a secondary antibody. Anti-hFIX antibody levels were measured following incubation with a detection buffer constituting 12 mL 0.01M sodium citrate, 12 mg o-phenylenediamine and 2.5 μL hydrogen peroxide (Invitrogen, Europe). The colorimetric reaction was monitored by determining the absorbance at 450 nm.

The results show that the majority of FIX-deficient hemophilia B mice treated with the AAV-AAT-modified FIX (SEQ ID NO: 394) did not develop any anti-FIX antibody response. At weeks 9 and 20, anti-FIX titer was low or comparable to vehicle-injected controls and substantially lower than the positive control for both FIX variants, indicating low immunogenicity of modified FIX (SEQ ID NO: 394) and Padua-FIX (SEQ ID NO: 488) after AAV-based gene therapy.

To assess for liver toxicity, aspartate aminotransferase (AST) and alanine transaminase (ALT) activity were determined in plasma using AST (MAK055-1KT, Sigma Aldrich, MO, USA) and ALT activity assay kits (MAK052-1KT, Sigma Aldrich, MO, USA), according to the manufacturer's instructions. At weeks one and three post vector administration, AST and ALT levels in plasma were determined for AAV-modified FIX (SEQ ID NO: 394) vector at the 4 E+10, 2 E+11 or 4 E+11 vg/kg dose levels. In normal healthy mice, the expected levels of ALT are up to 60 mU/mL and AST falls in the range of 50-100 mU/mL.

The liver toxicity results show that AST and ALT levels were within normal parameters (defined above) after injecting the FIX-deficient mice with AAV-modified FIX (SEQ ID NO: 394) vector. While still within the normal range, a slight and transient increase in AST and ALT levels was observed at one week post vector administration in those cohorts receiving the mid-dose (2 E+11 vg/kg) and high dose (4 E+11 vg/kg) of AAV-modified FIX (SEQ ID NO:394) and AAV-Padua (SEQ ID NO:488). The effect was transient, as by week three AST and ALT levels were comparable to vehicle-injected control mice. In the low dose cohorts, no increase in AST or ALT levels was observed.

### Example 14

### FIX Protein Expression and Activity in Mouse Plasma after Injection of a Recombinant Adeno-Associated Viral Vector (rAAV), Provided Herein, Encoding Modified FIX, Padua FIX, and WT-FIX

For this example, the modified FIX, Padua FIX, and WT-FIX, contain the residue T at position 148. As above, modified FIX, Padua FIX, and WT-FIX refer to the FIX whose mature forms are set forth in SEQ ID NOs:394, 488, and 3, with a modification of T at residue 148 (SEQ ID NOs: 489, 491, 490), respectively.

In vivo transduction efficiency of each rAAV packaged with the capsid designated KP1 (SEQ ID NO:418), and expressing modified FIX, Padua FIX, or wild-type FIX, was examined in mice. C57BL/6 FIX-deficient mice were injected with rAAV FIX vector packaged with the capsid KP1; FIX expression and coagulation activity were monitored over several weeks.

A. FIX Protein Expression in Plasma after Intravenous Injection of Modified FIX, Padua FIX and WT-FIX-rAAV in a Mouse Model of Hemophilia B

rAAV FIX vector expressing either modified FIX, Padua FIX, or wild-type FIX was produced as detailed above in Example 10. C57BL/6 F9-deficient mice (3-5 mice/group) were injected via the tail vein with either 8 E+11, 8 E+10 or 8 E+9 vector genomes per kg (vg/kg) assuming a nominal mouse weight of 25 grams of rAAV FIX vector expressing either modified, Padua FIX or wild-type FIX packaged in the capsid designated KP1 (SEQ ID NO:418). Blood was collected from the retro orbital plexus at weeks 1, 2, 3, 4, 5, 7, 9, 12, 14 and 17 post-intravenous dosing and plasma was prepared for bioanalytical assays.

For blood collection from the retro orbital plexus, non-heparinized capillaries were rinsed with 3.8% sodium citrate and used for blood collection. Sodium citrate was added to blood collected to 0.4% of final. Plasma was prepared by centrifugation at 10,000 RPM at 4° C. for ten minutes and treated blood samples were stored at −80° C. Blood and plasma samples were kept on ice throughout collection and processing.

The FIX concentration in plasma was determined using a FIX enzyme-linked immunosorbent antigen assay (ELISA). Briefly, wells of a 96-well plate were coated with 2 μg/mL of anti-human Factor IX antibody (AHIX-5041, Heamatologic Technologies, Essex VT) to capture the FIX. Detection of the captured FIX was performed with a goat anti-human FIX polyclonal antibody conjugated with HRP (GAFIX-HRP, Affinity Biologicals, Ontario Canada), at 2 μg/mL. Upon binding of FIX to the FIX antibody, a colorimetric signal was emitted, wherein signal strength is directly proportional to the quantity of FIX. The colorimetric reaction was measured on a Spectra MAX UV/VIS with SOFTmax PRO (Molecular Devices, San Jose, Calif.) and the unknown FIX concentrations in plasma were interpolated from a standard curve ranging from 0.4 ng/mL to 800 ng/mL of recombinantly expressed variant, Padua or wild-type FIX.

Data are provided in Table 48, below. The results show that no significant differences in plasma FIX levels were observed among modified FIX, Padua FIX and wild-type FIX. The levels observed are stable for up to 17 weeks and equivalent to approximately 5-10% of normal mice when using 8 E+11 vg/kg vector injection of AAV-FIX. Vector injections at lower doses (8 E+10 and 8 E+9 vg/kg) led to lower plasma FIX levels reaching a plateau within two weeks and remained stable through week 7.

TABLE 48
modified FIX, Padua FIX and wild-type FIX levels in plasma after 8E+11, 8E+10
and 8E+9 vector genomes per kg administered intravenously via the tail vein
(values are ± standard deviation; nd = not determined; FIX* is the
modified FIX whose mature sequence is set forth in SEQ ID NO: 394).

| | Average levels of FIX (μg/mL) | | | Average levels of FIX (μg/mL) | | | Average levels of FIX (μg/mL) | | |
|---|---|---|---|---|---|---|---|---|---|
| | (8E+11 vg/kg) | | | (8E+10 vg/kg) | | | (8E+9 vg/kg) | | |
| | | FIX- | FIX- | | FIX- | FIX- | | FIX- | FIX- |
| Week | FIX* | Padua | WT | FIX* | Padua | WT | FIX* | Padua | WT |
| 1 | 26.53 ± | 26.87 ± | 20.66 ± | 3.66 ± | 2.96 ± | 1.97 ± | 0.16 ± | 0.19 ± | 0.11 ± |
| | 3.67 | 4.16 | 1.03 | 0.76 | 0.49 | 0.75 | 0.01 | 0.11 | 0.03 |
| 2 | 26.91 ± | 23.52 ± | 16.98 ± | 4.28 ± | 3.94 ± | 2.60 ± | 0.19 ± | 0.22 ± | 0.13 ± |
| | 3.36 | 3.49 | 2.82 | 0.52 | 1.96 | 0.03 | 0.02 | 0.10 | 0.02 |
| 3 | 20.54 ± | 19.66 ± | 15.13 ± | nd | nd | nd | nd | nd | nd |
| | 2.94 | 4.74 | 1.69 | | | | | | |
| 4 | 17.91 ± | 19.70 ± | 15.17 ± | 4.98 ± | 4.38 ± | 2.95 ± | 0.23 ± | 0.25 ± | 0.15 ± |
| | 3.89 | 3.13 | 2.53 | 0.50 | 1.63 | 0.45 | 0.02 | 0.14 | 0.04 |
| 5 | 17.04 ± | 18.39 ± | 13.10 ± | nd | nd | nd | nd | nd | nd |
| | 2.30 | 3.30 | 2.70 | | | | | | |
| 7 | 14.33 ± | 16.23 ± | 11.47 ± | 4.46 ± | 3.21 ± | 2.18 ± | 0.25 ± | 0.25 ± | 0.13 ± |
| | 2.73 | 1.01 | 1.25 | 0.70 | 0.57 | 0.45 | 0.05 | 0.14 | 0.03 |
| 9 | 15.06 ± | 15.17 ± | 12.85 ± | nd | nd | nd | nd | nd | nd |
| | 3.79 | 2.47 | 1.77 | | | | | | |
| 12 | 12.14 ± | 14.77 ± | 11.13 ± | nd | nd | nd | nd | nd | nd |
| | 0.71 | 2.50 | 1.33 | | | | | | |
| 14 | 13.16 ± | 13.23 ± | 10.65 ± | nd | nd | nd | nd | nd | nd |
| | 2.10 | 3.12 | 1.06 | | | | | | |
| 17 | 13.07 ± | 14.12 ± | 10.67 ± | nd | nd | nd | nd | nd | nd |
| | 3.05 | 2.38 | 2.22 | | | | | | |

B. FIX Activity in Plasma after Intravenous Injection of Modified FIX, Padua FIX and WT-FIX-rAAV in a Mouse Model of Hemophilia B

rAAV FIX vectors expressing either modified FIX, Padua FIX, or wild-type FIX were produced as detailed above in Example 10. C57BL/6 FIX-deficient mice (3-5 mice/group) were injected via the tail vein with either 8 E+11, 8 E+10 or 8 E+9 vector genomes per kg (vg/kg) assuming a nominal mouse weight of 25 grams of rAAV FIX vector expressing either modified FIX, Padua FIX, or wild-type FIX. Blood was collected retro-orbitally at weeks 1, 2, 3, 4, 5, 7, 9, 12, 14 and 17 post-intravenous dosing and plasma was prepared for examination of the functional coagulation activity for each of the modified FIX, Padua FIX, and wild-type FIX cohorts.

FIX activity was assessed using an activated partial thromboplastin time (aPTT)-based factor IX single-stage clotting assay. The aPTT-assay was performed on an ACL-TOP instrument (Instrumentation Laboratories (Bedford, Mass.)) using indicated reagents and protocols. Briefly, an aPTT reagent, sold as HemosIL® or SynthasIL was equilibrated at 37° C. and pre-incubated with dilutions of mouse plasma harvested at various time points from mice treated with the rAAV, according to the manufacturer's instructions. Clotting was induced through the automated addition of 20 mM calcium chloride, which triggers the coagulation process. The resultant clot was detected by measuring the change in optical density. The amount of time required for the plasma specimen to clot also was recorded. Calibration was performed with the HemosIL Calibration Plasma (Instrumentation Laboratories, Bedford, Mass.) as a reference traceable to the WHO standard (09/172). Each sample was run in duplicate and using the required number of dilutions to obtain valid and reportable results. Sample dilutions ranged from 1:10 to 1:300, depending on anticipated FIX expression levels.

Data are provided in Table 49, below. The results show that FIX activity, as determined by the activated partial thromboplastin time (aPTT)-assay, was restored with wild-type FIX. Modified FIX and Padua FIX show approximately 15- and 8-fold increases in activity, respectively, compared to wild-type FIX. Thus, modified FIX and Padua FIX showed dramatically increased activity compared to this baseline level of wild-type FIX when the AAV is packaged with the capsid of SEQ ID NO: 418, and at levels approximately 10-fold higher than FIX packaged with the previously characterized DJ8 capsid (see, Example 12 and Table 45, above).

TABLE 49
Modified FIX, Padua FIX, and wild-type FIX activity in plasma after 8E+11,
8E+10 and 8E+9 vector genomes per kg administered intravenously
via tail vein (values are ± standard deviation; nd = not determined).

| | FIX activity in U/mL | | | FIX activity in U/mL | | | FIX activity in U/mL | | |
|---|---|---|---|---|---|---|---|---|---|
| | (8E+11 vg/kg) | | | (8E+10 vg/kg) | | | (8E+9 vg/kg) | | |
| | | FIX- | FIX- | | FIX- | FIX- | | FIX- | FIX- |
| Week | FIX* | Padua | WT | FIX* | Padua | WT | FIX* | Padua | WT |
| 1 | 129.73 ± | 63.47 ± | 7.74 ± | 16.36 ± | 7.81 ± | 1.03 ± | 0.71 ± | 0.53 ± | 0.13 ± |
| | 21.58 | 12.52 | 0.86 | 3.12 | 1.89 | 0.27 | 0.04 | 0.22 | 0.01 |
| 2 | 131.14 ±± | 61.09 ± | 6.82 ± | 19.21 ± | 9.59 ± | 1.19 ± | 0.85 ± | 0.60 ± | 0.16 ± |
| | 19.94 | 11.34 | 1.52 | 2.13 | 4.24 | 0.05 | 0.11 | 0.25 | 0.02 |
| 3 | 109.24 ± | 45.36 ± | 6.02 ± | nd | nd | nd | nd | nd | nd |
| | 15.09 | 9.56 | 0.6 | | | | | | |
| 4 | 81.02 ± | 44.22 ± | 5.95 ± | 22.99 ± | 10.26 ± | 1.40 ± | 0.84 ± | 0.67 ± | 0.17 ± |
| | 11.71 | 6.09 | 1.08 | 3.04 | 2.68 | 0.25 | 0.07 | 0.31 | 0.02 |
| 5 | 88.71 ± | 48.35 ± | 5.26 ± | nd | nd | nd | nd | nd | nd |
| | 8.03 | 9.47 | 0.65 | | | | | | |
| 6.5 | nd | nd | nd | 22.29 ± | 9.28 ± | 1.17 ± | 0.76 ± | 0.44 ± | 0.10 ± |
| | | | | 3.28 | 1.55 | 0.32 | 0.10 | 0.25 | 0.01 |
| 7 | 66.50 ± | 39.84 ± | 5.09 ± | nd | nd | nd | nd | nd | nd |
| | 10.56 | 1.79 | 0.63 | | | | | | |
| 9 | 57.18 ± | 30.25 ± | 4.57 ± | nd | nd | nd | nd | nd | nd |
| | 8.38 | 4.32 | 0.65 | | | | | | |
| 11.5 | 55.78 ± | 33.64 ± | 4.07 ± | nd | nd | nd | nd | nd | nd |
| | 3.93 | 6.63 | 0.27 | | | | | | |
| 13.5 | 71.59± | 36.38 ± | 3.37 ± | nd | nd | nd | nd | nd | nd |
| | 10.43 | 9.80 | 0.76 | | | | | | |
| 16.5 | 66.79 ± | 43.49 ± | 5.48 ± | nd | nd | nd | nd | nd | nd |
| | 12.68 | 9.60 | 1.05 | | | | | | |

FIX* is the modified FIX whose mature sequence is set forth in SEQ ID NO: 394, with the 148A allele

C. FIX Specific Activity in Plasma after Intravenous Injection of Modified FIX, Padua FIX and WT-FIX-rAAV in a Mouse Model of Hemophilia B

The specific activity of FIX, modified FIX and Padua FIX was determined by the assessment of antigen levels and protein coagulation activity. The FIX-specific activity was calculated by dividing the clotting activity by the antigen levels and expressing the results in units per milligram.

Data are set forth in Table 50, below. The results show that the specific activity of modified FIX was consistently highest for modified FIX for all levels of vg administered. For example, modified FIX showed 12-14 times the specific activity of wild-type FIX after administration of 8 E+10 and 8 E+9 vg per animal of the AAV vector. This was greater than the specific activity of Padua FIX, which showed approximately 6 times the specific activity of wild-type FIX. Thus, modified FIX has dramatically improved FIX specific activity with the modified FIX set forth in SEQ ID NO: 394, and exceeds the previously characterized high activity Padua FIX (SEQ ID NO: 488).

TABLE 50
Modified FIX, Padua FIX and wild-type FIX activity in plasma after 8E+11, 8E+10
and 8E+9 vector genomes per kg administered intravenously via tail vein (values
are ± standard deviation; nd = not determined; FIX* is the modified
FIX whose mature sequence is set forth in SEQ ID NO: 394, except with the T148A allele).

| | FIX Specific Activity (U/μg) | | | FIX Specific Activity (U/μg) | | | FIX Specific Activity (U/μg) | | |
|---|---|---|---|---|---|---|---|---|---|
| | (8E+11 vg/kg) | | | (8E+10 vg/kg) | | | (8E+9 vg/kg) | | |
| | | FIX- | FIX- | | FIX- | FIX- | | FIX- | FIX- |
| Week | FIX* | Padua | WT | FIX* | Padua | WT | FIX* | Padua | WT |
| 1 | 5.20 ± | 2.36 ± | 0.37 ± | 4.48 ± | 2.62 ± | 0.55 ± | 4.56 ± | 2.88 ± | 1.27 ± |
| | 0.37 | 0.29 | 0.03 | 0.17 | 0.19 | 0.09 | 0.15 | 0.47 | 0.24 |
| 2 | 5.20 ± | 2.45 ± | 0.37 ± | 4.49 ± | 2.48 ± | 0.46 ± | 4.57 ± | 2.67 ± | 1.24 ± |
| | 0.47 | 0.29 | 0.02 | 0.18 | 0.17 | 0.02 | 0.26 | 0.06 | 0.16 |
| 3 | 5.41 ± | 2.33 ± | 0.40 ± | nd | nd | nd | nd | nd | nd |
| | 0.59 | 0.20 | 0.03 | | | | | | |
| 4 | 4.58 ± | 2.26 ± | 0.39 ± | 4.69 ± | 2.40 ± | 0.48 ± | 3.71 ± | 2.80 ± | 1.28 ± |
| | 0.47 | 0.26 | 0.03 | 1.09 | 0.26 | 0.04 | 0.11 | 0.35 | 0.53 |
| 5 | 5.25 ± | 2.63 ± | 0.41 ± | nd | nd | nd | nd | nd | nd |
| | 0.56 | 0.20 | 0.04 | | | | | | |
| 6.5 | nd | nd | nd | 5.01 ± | 2.90 ± | 0.53 ± | 3.02 ± | 1.70 ± | 0.72 ± |
| | | | | 0.11 | 0.06 | 0.05 | 0.15 | 0.01 | 0.08 |
| 7 | 4.66 ± | 2.46 ± | 0.44 ± | nd | nd | nd | nd | nd | nd |
| | 0.30 | 0.12 | 0.03 | | | | | | |
| 9 | 3.91 ± | 2.00 ± | 0.36 ± | nd | nd | nd | nd | nd | nd |
| | 0.67 | 0.06 | 0.04 | | | | | | |
| 11.5 | 4.60 ±± | 2.28 ± | 0.37 ± | nd | nd | nd | nd | nd | nd |
| | 0.38 | 0.28 | 0.03 | | | | | | |
| 13.5 | 5.47 ± | 2.75 ± | 0.31 ± | nd | nd | nd | nd | nd | nd |
| | 0.44 | 0.29 | 0.05 | | | | | | |
| 16.5 | 5.15 ± | 3.07 ± | 0.52 ± | nd | nd | nd | nd | nd | nd |
| | 0.27 | 0 32 | 0 06 | | | | | | |

The experiments detailed above, with results set forth in Tables 48, 49 and 50, were continued for additional time, and FIX expression, activity and specific activity, were measured. The results show that FIX expression and activity at the later time point follow the trends of the earlier time points. For example, mice administered 8 E+11 vg/kg of modified FIX, Padua FIX, or wild-type FIX, showed stable FIX levels at 18 weeks post injection. Mice administered 8 E+10 vg/kg and 8 E+9 vg/kg FIX showed stable FIX levels for up to 12 weeks, with higher expression of modified FIX, and Padua FIX, compared to wild-type FIX. FIX activity and specific activity also were tracked for 18 weeks in mice administered 8 E+11 vg/kg FIX, and for 12 weeks in mice administered 8 E+10 vg/kg and 8 E+9 vg/kg FIX. The results show that FIX activity and specific activity, in all groups, was maintained at the 18 week and 10 week time points, with modified FIX showing the highest activity at all of 8 E+11, 8 E+10 and 8 E+9 vg/kg, followed by Padua FIX. Modified FIX and Padua FIX showed significantly higher activity than wild-type FIX at all time points, through 12 weeks for mice administered 8 E+10 and 8 E+9 vg/kg, and through 18 weeks in mice administered 8 E+11 vg/kg. Specific huFIX activity in animals injected with modified FIX expressing rAAV was around 10-fold or 2-fold enhanced as compared to those injected with wild type or Padua respectively (8×10″ and 8×10⁹ groups).

### D. Conclusions

The in vivo performances of the three FIX constructs: the modified FIX (mature sequence designated CB2769 and set forth in SEQ ID NO:394, except that residue 148 is the A allele), Padua FIX, and wild-type FIX demonstrate stabilizing antigen levels by week three that were ˜10-fold higher than observed using the DJ/8 capsid (Example above). The new rAAV expression cassette in conjunction with the KP1 capsid was shown to exhibit robust and very strong huFIX expression after injection into hemophilic mice. FIX activity levels were significantly increased for the modified variants, modified FIX and Padua, compared to the wild-type transgene at week three (109 IU/mL and 45 IU/mL versus 6 IU/mL for modified FIX, Padua, and wild-type respectively at the 8 E+11 vg/kg dose). In accord with the antigen data, the observed FIX activity levels for modified FIX were more than 10-fold higher than the activity levels using the DJ/8 (Example 13, above, and Example 16, below) vector at comparable doses. Comparative data are as follows:

| | | | FIX Activity |
|---|---|---|---|
| FIX | Capsid | Dose | (U/mL) |
| Modified FIX | KP1 | 8.0E+10 | 20 |
| R338L | TAK-748* | 7.4E+11 | 20 |
| R338L | TAK-748* | 7.4E+11 | 1 |
| Modified FIX | DJ8 | 2.0E+11 | 4 |
| Modified FIX | DJ8 | 2.0E10 | 1 |

*Weller et al.(2019) Blood 134: S1 P4633; see, also Example 15

These results demonstrate that the combination of the chimeric capsid and modified FIX that combines increased coagulation activity, resistance to endogenous inhibitor, and increased FVIII binding provides a striking increase in FIX activity levels.

Therefore, the combination of the AAV-vectors described herein for encoding and delivering modified FIX that have enhanced potency, such at least about 7, 10, or 20-fold higher than wild-type exhibits, and as constructed with an intron, provide improved transgene expression in vivo. The vectors and constructs provided herein, can be administered with lower the viral doses than other current rAVV-FIX gene therapy vectors, to achieve FIX activity levels for treating or essentially curing hemophilia in treated individuals.

### Example 15

### Evaluation of the Human Factor IX Gene Therapy Vector TAK-748 (SHP648)

### COMPARATIVE EXAMPLE

FIX protein expression and activity in mouse plasma after intravenous injection of a recombinant adeno-associated viral vector (rAAV) TAK-748 encoding and expressing Padua FIX (with the single replacement R338L) is described, for comparison with Example 14. For comparison with the rAAV vectors and constructs provided herein, in vivo transduction efficiency of rAAV TAK-748 (Baxalta US Inc., a Takeda company, Lexington, Mass., USA) and expression of the modified FIX with the replacement R338L (SEQ ID NO: 488) was examined in mice.

### A. TAK-748 Vector (Previously Designated SHP-648)

TAK-748 is a single stranded AAV vector, based on AAV8, that includes the insertion of 3 hepatocyte-specific cis-regulatory elements (CRM8) to increase the strength of the liver-specific transthyretin (TTR) promoter driving expression of the Padua variant human FIX transgene. The TTR enhancer promoter delivers liver-restricted expression of the transgene. The FIX transgene (encoding the modified FIX set forth in SEQ ID NO: 488) is a cytosine-phosphate-guanosine (CpG)-depleted FIX variant. This codon optimized CpG-depleted nucleotide sequence was designed to increase FIX expression and decrease potential immunogenicity.

B. FIX R338L Activity after Intravenous Injection of FIX Gene Therapy Vector TAK748 in a Mouse Model of Hemophilia B

Male FIX KO mice (N=12/group) received a single intravenous dose of TAK-748 7.4 E+10, 1.5 E+11, 7.4 E+11, or 1.5 E+12 vector genomes [vg]/kg, or buffer as a negative control. FIX expression, blood clotting activity, transduction efficiency and safety were monitored over several weeks.

FIX R338 L Activity

Blood was collected on days 3, 7, 14, 28, 42, 56 and 84 and FIX activity in plasma was analyzed using a one-stage clotting assay. The results show that TAK-748 administration increased the mean FIX activity in FIX knock-out mice plasma in a dose-dependent manner. Administration of 1.5 E+12 vg/kg increased mean FIX activity to supraphysiologic levels of up to 41.0 IU/mL. FIX activity levels in plasma from control FIX knockout (KO) mice treated with buffer were below the lower limit of quantification.

Blood loss also was assessed using a tail-tip bleeding assay. The results show that mean blood loss in buffer control was approximately 30 mg/g. In the animals administered the TAK-748 FIX vector, blood loss was reduced in a dose dependent manner. Blood loss was significantly reduced in groups administered 1.5 E+11, 7.4 E+11, or 1.5 E+12 vector genomes (vg/kg) compared to the buffer control (P<0.05).

Transduction Efficiency

The viral transduction efficiency of TAK-748 in liver tissue was analyzed by quantitative real-time polymerase chain reaction (qPCR) and histological analysis. The results show a dose dependent increase in FIX copies per cell. For example, there was approximately 2-fold more copies of FIX per cell after administration of 7.4 E+11 compared to 1.5 E+12. Similar results were demonstrated after immunohistochemical analysis, which showed a dose-dependent increase in FIX expression in liver after TAK-748 administration.

Safety Assessments

Animals were monitored for clinical and histopathological adverse effects. Selected organs (liver, spleen, kidney, and heart) were analyzed. The results show that no clinical histopathological or signs, or premature deaths were recorded in animals treated with TAK-748.

C. FIX R338L Activity after Intravenous Injection of FIX Gene Therapy Vector TAK748 in Rhesus Monkeys

Male rhesus monkeys (N=3/group) were administered a single intravenous bolus injection of TAK-748-FIX at 3.8 E+11, 9.5 E+11, or 1.9 E+12 vg/kg. Blood samples were collected prior to dosing and weekly thereafter, until week 18. Plasma FIX activity, human (hu)FIX antigen, and anti-hu-v FIX neutralizing antibodies were analyzed.

FIX R338L Activity

Blood was collected weekly for 12 weeks and FIX activity in plasma was analyzed using a one-stage clotting assay. The results show that TAK-748 administration to rhesus monkeys resulted in a dose-dependent increase in mean plasma FIX activity and antigen. Peak levels of hu-FIX R338L (SEQ ID NO: 488) expression were detected 2-4 weeks after treatment. Mean hu-FIX activity was 0.3, 0.6, and 1.9 IU/mL after treatment with 3.8 E+11 vg/kg, 9.5 E+11 vg/kg, and 1.9 E+12 vg/kg TAK-748, respectively. A significant reduction in FIX activity and huFIX protein was observed in most animals beginning approximately 4 weeks after dosing. In most animals, anti-huFIX neutralizing antibody titers were detected at approximately week 6 and correlated with decreased FIX R338L (SEQ ID NO: 488) expression.

Safety Assessments

Animals were monitored for clinical adverse effects. No adverse clinical effects were observed.

### Example 16

### Modified FIX Variants Tested in Mouse Model in AAV Vector Packaged in the Capsid Designated DJ8

This example repeats experiments described in Example 13; and confirms the previous results in hemophilic mice cohorts, with a vector batch produced de novo, and using two vector doses. Assessments were conducted via modified phenotypic and analysis assays. FIX protein expression and activity in mouse plasma after injection of the recombinant adeno-associated viral vector (rAAV) encoding the modified FIX (mature form encoded by the nucleic acid sequence set forth in SEQ ID NO:490, or SEQ ID NO:394, except that the replacements are in the FIX allele (SEQ ID NO:481) in which residue 148 is A, for expression and packaging in the capsid designated DJ8 (SEQ ID NO:427; Bio-connect, The Netherlands; Grimm et al., J Virol 82:5887-911 (2008)). As in Example 13, the nucleic acid encoding the mature FIX is codon optimized for expression in mice. Expression of the encoded modified FIX was analyzed for comparison with results in Example 14, above. The data show that the use of the capsids, described herein, that have increased tropism for hepatocytes compared to vectors packaged in the capsid DJ/8, as well as including the FIX intron, increases the amount of FIX expressed and the resulting coagulation activity.

C57BL/6 FIX-deficient mice were injected with rAAV FIX vector packaged with the DJ8 capsid, and FIX expression and coagulation activity was monitored for several weeks. In these experiments, the phenotypic assays to assess FIX activity were modified to demonstrate the difference in biological efficacy between the modified FIX (R318Y, T343R, R338E, T148A) and the R338L FIX (Padua), by assessing clotting time, blood volume loss, and bleeding time.

A. Cloning of the FIX Gene and rAAV Vector Production

The FIX gene was cloned and rAAV vectors were prepared as detailed in Example 11, above. Research grade AAV/DJ8 vectors were produced by calcium phosphate co-transfection of HEK-293 human embryonic kidney carcinoma cells with the pAAV plasmid, an adenoviral helper plasmid and chimeric packaging construct that delivers the AAV2 Rep gene and the AAV/DJ8 capsid gene. The AAV particles were purified. Known copy numbers (102-108) of the vector plasmids used to generate the corresponding AAV vectors, carrying the appropriate cDNA were used to generate the standard curves. Purified high-titer AAV vectors were obtained for both vectors modified FIX (R318Y, T343R, R338E, T148A) and the R338L FIX (Padua), at comparable levels, as detailed below:

TABLE 51
Summary of Vector Titers

| | Vol | size | Titer 1 | Titer 2 | Titer 3 | Titer 4 | Avg titer |
|---|---|---|---|---|---|---|---|
| Vector | (μL) | (bp) | (vg/mL) | (vg/mL) | (vg/mL) | (vg/mL) | (vg/mL) |
| FIX-R338L | 300 | 6270 | 7.1E+12 | 6.7E+12 | 7.71E+12 | 5.25E+12 | 6.67E+12 |
| Modified FIX | 305 | 6270 | 1.3E+13 | 1.1E+13 | 9.63E+12 | 9.83E+12 | 1.08E+13 |
| (R318Y, R338E, | | | | | | | |
| T343R, T148A) | | | | | | | |

B. FIX Protein Expression in Plasma after Intravenous Injection of rAAV-Modified FIX and Padua FIX in a Mouse Model of Hemophilia B

A colony of FIX hemophilic mice was established and additional breeding for the studies was implemented. The in vivo performances of the two FIX vectors at two difference doses were assessed. The design of the studies was as follows:

Mouse model - Male FIX-KO mouse - 5-6 week-old

| Vector/Condition | Dose 1 | Dose 2 |
|---|---|---|
| rAAV/DJ8sc-AAT-MVM- | 5 × 10⁰⁹ vg/mouse* | 1 × 10¹⁰ vg/mouse* |
| FIXcoPadua-bghpA | 4 mice | 4 mice |
| rAAV/DJ8sc-AAT-MVM- | 5 × 10⁰⁹ vg/mouse* | 1 × 10¹⁰ vg/mouse* |
| Fixico | | |
| (R318Y, R338E, T343R, | 4 mice | 4 mice |
| T148A)-bghpA | | |
| PBS | 4 mice | 4 mice |

Assuming the average mouse weighs about .025 kg (.75 ounces), 5 × 10⁹ vg/mouse is 2 × 10¹¹ vg/kg, and 1 × 10¹⁰ vg/mouse is 4 × 10¹¹ vg/kg

C57BL/6 F9-deficient mice (4 mice/group) were injected in the tail vein with either 2 E+11 or 4 E+11 vector genomes per kg (vg/kg) of rAAV FIX vector for expressing either modified FIX or Padua FIX, or PBS as a negative control. Whole blood was collected by phlebotomy of the retro orbital plexus at 1, 3, 5, 7, 8, 9, 12, 16 and 20 weeks post-intravenous dosing and plasma was prepared for bioanalytical assays.

Blood collection was performed at different times post vector injection into these mice. Blood was collected in 1.5 mL Eppendorf tubes containing 20% citrate buffer using non-heparinized capillaries. To obtain the plasma, the blood was centrifuged for 3 min at 13000 rpm. The plasma was aliquoted into 3 tubes, placed on dry ice and stored at −80° C. FIX antigen levels were determined on plasma samples from the mice injected with the vectors and non-injected controls by enzyme-linked immunosorbent assay (Asserachrome IX: Ag Enzyme Immunoassay for Factor FIX; Diagnostica Stago, France) using known concentrations of purified hFIX-R338L-Padua and modified FIX (R318Y, R338E, T343R, T148A) proteins, as respective standards. The FIX activity was measured using an aPTT test as per the manufacturer's instructions (C.K.PREST kit & Start Max; Stago). Plasma from non-injected mice was used as negative control.

A tail-clipping assay was performed to assess the phenotypic correction. Mice were anesthetized and the tail was placed in pre-warmed 37° C. normal saline solution for 2 minutes and subsequently cut at 2.5-3 mm diameter. Tail was then immediately placed in 37° C. normal saline solution and monitored for bleeding or clotting for 30 minutes. Blood-containing saline was centrifuged at 520 g for 15 min at 4° C. to collect erythrocytes and resuspended in 18 ml of lysis buffer (10 mM KHCO3, 150 mM NH4Cl, 0.1 mM EDTA). Lysis proceeded for 10 minutes at room temperature and samples were centrifuged at 520 g for 10 min at 4° C. OD at 570 nm of supernatants was measured. After the tail clip assay, mice were euthanized, and liver samples were collected from them for further RNA and DNA analysis (ongoing).

Results

At both the doses, at the 2 month time point post injection, the modified FIX (R318Y, R338E, T343R; T148A allele) showed a statistically significant 1.1-1.2× improvement in the clotting time compared to FIX Padua (R338L), but produced similar or 1.2× lower (p<0.05) amounts of FIX protein. Taking into consideration the difference in protein levels for the higher dose, then the overall activity resulted in an apparent 1.45 fold improvement when comparing modified FIX (R318Y, R338E, T343R, T148A) with FIX Padua (R338L) under the aPTT assay conditions defined above and a single volume of mouse plasma.

The results of the tail clip experiment show that, when both vector doses tested for phenotypic correction, the modified FIX (R318Y, R338E, T343R, in the T148A allele) had a statistically significant improvement in the bleeding time and blood loss. The difference in the blood loss is more apparent in the modified tail clip protocol, where the mice are bled for 30 min instead of 10 min.

C. FIX Activity in Plasma after Intravenous Injection of rAAV-Modified FIX and Padua FIX in a Mouse Model of Hemophilia B

rAAV-DJ8 FIX vectors encoding the modified FIX (SEQ ID NO: 490; containing the replacements R318Y, R338E, T343R in the T148A allele) and Padua FIX (FIX R338L) were produced as detailed above in Section A and in previous examples. C57BL/6 FIX-deficient mice (4 mice/group) were injected via the tail vein with either 2 E+11 or 4 E+11 vector genomes per kg (vg/kg) of rAAV-DJ8 FIX vector expressing modified FIX or Padua FIX. Two months post injection, blood was collected retro-orbitally, and plasma was prepared for examination of the functional coagulation activity of the modified FIX and Padua FIX.

FIX activity levels were determined using an activated partial thromboplastin time (aPTT)-based Factor IX single-stage clotting assay as detailed above.

The results show that FIX activity, as determined by the aPTT-assay, was restored with modified FIX and Padua FIX expression. The modified FIX (R318Y, R338E, T343R, T148A) and Padua FIX (R338L) showed a significant reduction in clotting times compared to vehicle control at both 2 E+11 or 4 E+11 vg/kg. The modified FIX (R318Y, R338E, T343R, T148A) showed a significantly lower clotting time (p<0.05) than Padua FIX (R338L) at both 2 E+11 or 4 E+11 vg/kg dose levels, in spite of producing similar or 1.2 times less FIX protein. After accounting for the difference in protein expression, modified FIX decreased clotting time by approximately 1.45 fold compared to Padua FIX.

Phenotypic efficacy was assessed using a modified mouse tail-clip assay and recording the blood loss and the bleeding time. Briefly, the mice were anesthetized, and the tails were placed in a pre-warmed saline solution for two minutes and subsequently cut at a 2.5-3 mm diameter. The tails were then immediately returned to the 37° C. saline solution and the bleeding time monitored for 30 minutes. Blood-containing saline was centrifuged at 520 g for 15 minutes at room temperature to collect erythrocytes, which were then resuspended in 18 mL of lysis buffer (10 mM KHCO3, 150 mM NH₄Cl, 0.1 mM EDTA). Lysis proceeded for 10 minutes at room temperature and samples were centrifuged again, at 520 g for 10 minutes at 4° C. The absorbance of the supernatants was measured at 570 nm spectroscopically to determine the amount of hemoglobin as an indication of blood loss.

The results show that bleeding time was significantly reduced (p<0.001) for both doses of modified FIX and Padua FIX compared to vehicle control. Bleeding time also was significantly reduced (p<0.001) in mice administered both doses of modified FIX compared to mice administered equivalent amounts of Padua FIX. For example, the bleeding time for mice administered 2 E+11 vg/kg modified FIX was approximately 4 minutes, compared to approximately 31 minutes for mice administered Padua FIX. The bleeding time for mice administered 4 E+11 vg/kg modified FIX was approximately 3 minutes, compared to approximately 16 minutes for mice administered Padua FIX. Both concentrations of modified FIX decreased the bleeding time significantly with a p<0.001 compared to the equivalent concentration of Padua FIX.

The phenotypic correction was performed for both the doses. The modified FIX (R318Y, R338E, T343R, (in the T148A allele)) is superior; it reduces bleeding time (5-8×) and blood loss (3.8-4.1×) compared to the FIX R338L. At both the doses, compared to the PBS control groups the FIX (R318Y, R338E, T343R, in the T148A allele) had a statistically significant decrease in the bleeding time (23 to 27-fold) and blood loss (8.7 to 8.9-fold). The FIX R338L showed a significant decrease of 2.7 to 5.7-fold in bleeding time and 2 to 2.3-fold in blood volume loss compared to the PBS control group. Thus, the vector with the modified capsid effectively delivers FIX; the modified FIX (R318Y, R338E, T343R) is superior to the FIX (R338L).

Blood loss volume also was significantly reduced in mice administered both rAAV-DJ8 FIX vectors, compared to vehicle control, with modified FIX (R318Y, R338E, T343R, in the T148A allele) inhibiting blood loss more effectively than Padua FIX (R338L). For example, mice administered 2 E+11 vg/kg modified FIX (R318Y, R338E, T343R, in the T148A allele)) lost significantly less blood than mice administered vehicle control (p<0.01) and mice administered 2 E+11 vg/kg Padua FIX (R338L) (p<0.001). Mice administered 4 E+11 vg/kg modified FIX (R318Y, R338E, T343R, in the T148A allele)) also lost significantly less blood than mice administered vehicle control (p<0.001) and mice administered 4 E+11 vg/kg Padua FIX (R338L) (p<0.01). The blood loss was reduced about 4-fold in mice receiving 2 E+11 and 4 E+11 vg/kg of modified FIX compared to Padua FIX. The results are set forth in Table 52 below:

TABLE 52
Average blood loss by testing hemoglobin levels (570 nm)

| Hemoglobin (570 nm) | | | Hemoglobin (570 nm) | | |
|---|---|---|---|---|---|
| (2E+11 vg/kg) | | | (4E+11 vg/kg) | | |
| | | Vehicle | | | Vehicle |
| Modified FIX* | FIX-Padua | (PBS) | FIX* | FIX-Padua | (PBS) |
| 0.04175 ± 0.0048 | 0.1725 ± 0.03 | 0.34225 ± 0.12 | 0.0385 ± 0.003 | 0.1485 ± 0.04 | 0.337 ± 0.08 |

*R318Y/R338E/T343 replacements in the allele with T148A

D. Conclusion

Mice administered AAV-DJ8-modified FIX (R318Y, R338E, T343R in the T148A allele; SEQ ID NO:490) and AAV-DJ8-Padua (FIX R338L) have high levels of FIX activity in the one-stage clotting assays following gene therapy, and both FIX proteins showed significantly increased FIX expression and activity compared to vehicle alone. The AAV-modified FIX (R318Y, R338E, T343R, in the T148A allele)) results in a superior reduction in blood loss and decreased bleeding time compared to AAV-FIX R338L. Independently, these two functional approaches confirm that modified FIX (R318Y, R338E, T343R, in the T148A allele)) exhibits superior hemostatic potency compared to Padua FIX (R338L).

### Example 17

### Vector Copy Numbers in Mouse Plasma after Injection of a Recombinant Adeno-Associated Viral Vector (rAAV) Packaged with KP1, Encoding the Modified FIX (R318Y/R338E/T343R), Padua FIX, or a WT-FIX

In vivo transduction efficiency of each rAAV packaged with the capsid designated KP1 (SEQ ID NO:418), and expressing modified FIX (R318Y/R338E/T343R), Padua FIX, or wild-type FIX, was examined in mice, as detailed in Example 15. C57BL/6 FIX-deficient mice were injected with each rAAV FIX vector packaged with the capsid KP1; huFIX transcript levels and rAAV vector geneome copy numbers were quantified in the livers of animals after sacrifice at week 18 (8 E+11 vg/kg group) or week 16 (8 E+9 vg/kg and 8 E+10 vg/kg groups) post rAAV injection.

The animals were sacrificed, livers were harvested, and the livers were prepared for analysis by qPCR as detailed above, in Example 13. Data are set forth in Tables 53 and 54, below, as mean±S.D. FIX expression is expressed as fold over actin baseline level. Three technical replicates of each sample were analyzed by qPCR. The results show huFIX transcript levels and AAV vector copy numbers were similar for all constructs, with the exception of animals administered 8 E+11 vg/kg modified (R318Y/R338E/T343R) FIX, which showed statistically significant higher vector copy numbers than wild-type FIX (p=0.025). One mouse from the Padua 8 E+9 vg/kg injection group died one day post injection. Data are set forth in Tables 53 and 54, below:

TABLE 53
Average AAV Vector Copy Numbers

| Vector Copy Number | | | Vector Copy Number | | | Vector Copy Number | | |
|---|---|---|---|---|---|---|---|---|
| (normalized) | | | (normalized) | | | (normalized) | | |
| (8E+11 vg/kg) | | | (8E+10 vg/kg) | | | (8E+9 vg/kg) | | |
| | FIX- | FIX- | | FIX- | FIX- | | FIX- | FIX- |
| FIX* | Padua | WT | FIX* | Padua | WT | FIX* | Padua | WT |
| 1.090 ± | 0.869 ± | 0.815 ± | 0.869 ± | 0.812 ± | 0.073 ± | 0.112 ± | 0.125 ± | 0.116 ± |
| 0.114 | 0.183 | 0.109 | 0.0071 | 0.0146 | 0.0141 | 0.0041 | 0.00297 | 0.0051 |

TABLE 54
FIX Expression

| Transcript levels | | | Transcript levels | | | Transcript levels | | |
|---|---|---|---|---|---|---|---|---|
| (8E+11 vg/kg) | | | (8E+10 vg/kg) | | | (8E+9 vg/kg) | | |
| | FIX- | FIX- | | FIX- | FIX- | | FIX- | FIX- |
| FIX* | Padua | WT | FIX* | Padua | WT | FIX* | Padua | WT |
| 7.180 ± | 6.504 ± | 5.854 ± | 2.214 ± | 3.025 ± | 1.645 ± | 0.0398 ± | 0.072 ± | 0.044 ± |
| 1.279 | 1.009 | 0.407 | 1.084 | 1.297 | 0.798 | 0.021 | 0.0217 | 0.006 |

The results show that rAAV vector copy numbers and FIX transcript levels were similar between wild-type FIX-, Padua FIX-, and modified FIX-rAAV-injected mice within the same dose groups. Animals injected with modified (R318Y/R338E/T343R) FIX rAAV had the highest huFIX activity levels for all three dose levels.

### Example 18

### FIX Protein Expression and Activity in Cynomolgus Monkey after Injection of a Recombinant Adeno-Associated Viral Vector (rAAV), rAAV-KP1, and the Vector Designated rAAV-LK03, Each Encoding Modified FIX (R318Y/R338E/T343R)

Expression and activity of modified Factor IX (R318Y/R338E/T343R replacements), intravenously delivered via the AAV-LK03 capsid (Lisowski et al., (2014) Nature 506(7488):382-386) or AAV-KP1 (SEQ ID NO:418), was assessed in blood plasma in a pilot study in cynomolgus monkeys. To test for safety of rAAV infusion, clinical chemistry, hematological assessments, and coagulation also were assessed.

rAAV-LK03 is a known AAV strain that has been characterized as a strain that efficiently and preferentially transduces human cells, and transduces primary human hepatocytes 100-fold better than AAV8 (Lisowski et al., (2014) Nature 506(7488):382-386; see, e.g., SEQ ID NO:561, which sets forth the LK03 capsid sequence (see, also, U.S. Patent Publication No. 2018/0135076 A1)). The expression of modified FIX, including the intron as described herein is encoded in LK03. AAV vectors containing the capsid LK03 is compared to the vectors containing KP1 capsid described herein. Expression of modified FIX in each of these vectors is compared.

As in Example 13, the nucleic acid encoding the mature FIX is codon optimized for expression in mice; the optimized codons are very similar (˜90%) to human optimized sequences. The data show that the capsids, described herein, that have increased tropism for human hepatocytes compared to vectors packaged in the capsid DJ/8, as well as including the FIX intron, achieved high initial FIX levels and coagulation activity while maintaining normal clinical chemistry levels and without negatively impacting the health or blood chemistry of the experimental animals.

A. Identification of Non-Human Primates (NHPs) with Low AAV Neutralization Antibodies Toward rAAV with Capsids LK03 or KP1

Activation of the immune systems in experimental animals can be an obstacle to efficient and safe in vivo gene transfer via AAV vectors (Mingozzi and High (2013) Blood 122(1):23-36). Anti-AAV neutralizing antibodies produced by experimental animals can impact transduction efficiency of intravenously delivered AAV (Masat et al. (2013) Discov. Med. 15(85):379-389). To address this, non-human primate (NHP) subjects were pre-screened before treatment with AAV to identify animals that have low levels of anti-AAV neutralizing antibodies.

To examine the natural production of AAV-neutralizing antibodies in NHPs, prior to treatment with AAV, serum from twelve cynomolgus monkeys was collected approximately twenty one days prior to AAV-KP1 and AAV-LK03 injection, and analyzed. Animals producing the lowest levels of AAV-neutralizing antibodies were selected for AAV injection to analyze FIX expression and activity. The procedure for analyzing AAV-neutralizing antibody production was modified from the protocol detailed in Meliani et al. (Human Gene Therapy Methods (2015) 26:45-53).

1. Control Experiments Test Lk03 And Kp1 In Vitro Transduction Efficiency

First, the AAV transduction efficiency of LK03 and KP1 was tested in SNU-387 Hepatocellular carcinoma cells. Cells were seeded on 96-well plates and the next day cells were transduced with 2, 20 or 200 MOI of LK03-luciferase or KP1-luciferase vectors essentially in accord with the protocol set forth in Meliani et al. (Human Gene Therapy Methods (2015) 26:45-53). Twenty-four hours after transduction, luciferase signal was measured. The results show luciferase signal, which is indicative of transduction efficiency, increased with increasing multiplicity of infection (MOI). The signal for LK03 and KP1 was not significantly different, and both vectors efficiently infected SNU-387 cells in these control experiments. The results are set forth as FLuc molecules (log₁₀) in Table 55, below:

TABLE 55
Transduction Efficiency of FLuc rAAV vectors
LK03-FIX and KP1-FIX on SNU-387 Cells

| | LK03 | KP1 |
|---|---|---|
| MOI 200 | 8.17 | 8.535 |
| MOI 20 | 6.86 | 7.29 |
| MOI 2 | 5.66 | 6.23 |

Next, pooled human intravenous immunoglobulin (IVIG) was used as a control/standard for neutralization of the AAVs LK03 and KP1. AAVs LK03-Luc and KP1-Luc were subject to serial dilution of the IVIG standard control to assess antibody neutralization of the AAV. Data are expressed, in Table 56, as percent luciferase expression normalized to luciferase expression in the absence of IVIG (set to 100, no neutralization). The results show that LK03 and KP1 are neutralized with increasing concentrations of IVIG, as demonstrated by decreasing luciferase expression with increasing IVIG. LK03 is more susceptible to lower concentrations of IVIG, compared to KP 1. The results are set forth in Table 56, below:

TABLE 56
AAV Neutralization by Pooled Human IVIG

| IVIG (μg/mL) | LK03 | KP1 |
|---|---|---|
| 0 | 100 | 100 |
| 20 | 13.65 | 60.74 |
| 40 | 1.73 | 45.68 |
| 60 | 0.25 | 35.25 |
| 80 | 0.13 | 24.79 |
| 100 | 0.04 | 17.7 |
| 125 | 0.02 | 13.06 |
| 150 | 0.01 | 7.07 |
| 200 | 0.02 | 2.9 |
| 300 | 0.02 | 0.51 |

* data represented as percent luciferase expression normalized to luciferase expression in the absence of IVIG (set to 100, no neutralization)

2. AAV In Vitro Transduction in the Presence of NHP Serum

Next, transduction efficiency of AVVs KP1 and LK03 was assessed in SNU-387 cells in the presence of serum collected from 12 non-human primates (NHPs) to identify animals with low AAV neutralizing antibodies. Twenty one days prior to AAV injection, blood was collected from twelve female Cynomolgus monkeys, processed into serum, and monkey serum was heat-inactivated for 30 minutes at 56° C. Monkey sera was then serially diluted in the heat-inactivated FBS (undiluted, 1:2, 1:4, 1:8, 1:16, 1:32, 1:64, 1:128). Next, 30 μL (1.4 E+7 vg) of the AAV-luciferase vector (LK03 or KP1) was incubated with 30 μL of undiluted serum, or a serum dilution (1:2, 1:4, 1:8, 1:16, 1:32, 1:64, 1:128) or a no monkey serum negative control (FBS only) for 1 hour at 37° C. Following incubation, 22.5 μL of each serum-virus mixture was used to transduce SNU-387 cells in duplicates at a MOI of 200. SNU-387 cells had been seeded the day before at 2.5e4 cells/well in a 48 well plate.

After 24 hours of transfection, cells were rinsed with PBS and 100 μL of Passive Lysis buffer (Promega, Madison, Wis.; Cat. No. E194I) was added to each well. Next, cell lysates were collected, frozen, thawed, and centrifuged. After centrifugation, 20 μL of each clarified supernatant was extracted from the cell lysates and incubated with the luciferase substrate (Promega, Madison, Wis.; Cat. No. E1910) according to manufacturer's instructions, and luminescence was measured on a plate reader.

Two technical replicates of each sample dilution (or undiluted, or no serum control) were analyzed for luciferase luminescence. Data are expressed, in Tables 57 and 58, as percent luciferase expression normalized to luciferase expression in the absence of serum (set to 100, no neutralization).

The results show variability in the level of neutralizing antibodies among the different NHPs. When neutralizing antibodies (nAB) are present, low luciferase signal is detected in undiluted serum. Overall, there was a less of a neutralizing effect of pre-existing nAb observed with KP1 compared to LK03. For example, NHP3 had antibodies to LK03, but not to KP1. Animals with high levels of neutralizing antibodies (e.g., NHP2, 4, 8, 10, 11, and 12) were excluded from further study. Animals that showed lower levels of antibody neutralization antibodies (e.g., NHP1, 3, 5, 6, 7, and 9) were used for future experiments to determine the impact of rAAV administration on blood chemistry, coagulation, and FIX expression and activity, detailed below. NHPs 1, 5 and 7 were selected for experiments with rAAV-LK03, and NHPs 3, 6 and 9 were selected for experiments with rAAV-KP1.

Data are set forth in Tables 57 and 58, below, as a percentage of the no serum control (set to 100):

TABLE 57
Antibody Neutralization of AAV-KP1.

| | NHP Animal Identification Number | | | | | | | | | | | |
|---|---|---|---|---|---|---|---|---|---|---|---|---|
| Dilution | 1 | 2 | 3 | 4 | 5 | 6 | 7 | 8 | 9 | 10 | 11 | 12 |
| No serum | 100 | 100 | 100 | 100 | 100 | 100 | 100 | 100 | 100 | 100 | 100 | 100 |
| 1:128 | 88.82 | 41.87 | 81.38 | 76.56 | 82.97 | 79.54 | 77.52 | 1.23 | 85.18 | 26.81 | 85.74 | 68.35 |
| 1:64 | 92.60 | 19.85 | 102.98 | 60.96 | 92.13 | 85.90 | 88.71 | 0.04 | 91.95 | 9.21 | 88.14 | 61.45 |
| 1:32 | 103.51 | 1.75 | 105.43 | 33.79 | 105.28 | 66.13 | 75.86 | 0.02 | 83.43 | 1.25 | 61.60 | 32.71 |
| 1:16 | 84.62 | 0.05 | 105.09 | 6.73 | 96.11 | 49.34 | 80.08 | 0.01 | 88.10 | 0.13 | 34.22 | 5.27 |
| 1:8 | 79.60 | 0.01 | 108.66 | 0.30 | 99.45 | 23.03 | 83.33 | 0.01 | 84.87 | 0.01 | 11.30 | 0.27 |
| 1:4 | 50.43 | 0.01 | 114.50 | 0.01 | 97.59 | 5.68 | 86.78 | 0.01 | 97.49 | 0.01 | 1.28 | 0.01 |
| 1:2 | 22.63 | 0.01 | 111.44 | 0.01 | 102.79 | 0.66 | 95.16 | 0.01 | 92.52 | 0.01 | 0.17 | 0.01 |
| Undil. | 4.18 | 0.01 | 111.50 | 0.01 | 97.14 | 0.03 | 109.64 | 0.01 | 111.8 | 0.01 | 0.01 | 0.01 |

*NHP animals 3, 6, 9 were selected for further study (cells highlighted in bold, cols. 3, 6, and 9)

TABLE 58
Antibody Neutralization of AAV-LK03

| | NHP Animal Identification Number | | | | | | | | | | | |
|---|---|---|---|---|---|---|---|---|---|---|---|---|
| Dilution | 1 | 2 | 3 | 4 | 5 | 6 | 7 | 8 | 9 | 10 | 11 | 12 |
| No serum | 100 | 100 | 100 | 100 | 100 | 100 | 100 | 100 | 100 | 100 | 100 | 100 |
| 1:128 | 107.12 | 45.02 | 106.50 | 61.60 | 102.69 | 56.08 | 72.44 | 0.01 | 86.64 | 18.08 | 21.72 | 65.14 |
| 1:64 | 111.00 | 13.37 | 111.00 | 26.34 | 108.94 | 47.44 | 83.66 | 0.01 | 82.12 | 4.01 | 5.08 | 36.93 |
| 1:32 | 109.24 | 1.43 | 106.86 | 3.68 | 106.68 | 22.92 | 99.48 | 0.01 | 79.21 | 0.45 | 0.22 | 10.67 |
| 1:16 | 95.89 | 0.04 | 107.23 | 0.10 | 107.26 | 3.89 | 96.11 | 0.01 | 81.99 | 0.02 | 0.02 | 0.43 |
| 1:8 | 55.75 | 0.03 | 103.38 | 0.03 | 111.17 | 0.15 | 90.76 | 0.02 | 79.72 | 0.01 | 0.01 | 0.02 |
| 1:4 | 20.04 | 0.03 | 90.75 | 0.02 | 111.00 | 0.02 | 91.66 | 0.02 | 88.36 | 0.02 | 0.02 | 0.02 |
| 1:2 | 1.50 | 0.03 | 39.53 | 0.03 | 111.50 | 0.02 | 107.46 | 0.02 | 85.87 | 0.02 | 0.02 | 0.02 |
| Undil. | 0.11 | 0.03 | 7.37 | 0.03 | 110.00 | 0.03 | 112 | 0.03 | 82.89 | 0.02 | 0.02 | 0.02 |

*NHP animals 1, 5, 7 were selected for further study (cells highlighted in bold, cols 1, 5, and 7)

B. Study Comparing AAV Neutralization, Blood Chemistry, FIX Expression and FIX Activity in NHPs Administered rAAV LK03-FIX or rAAV-KP1 FIX

Animals that showed the lowest levels of AAV-neutralizing antibody production were selected for further experiments; non-human primates (NHP) designated 1, 5, and 7 were selected for intravenous infusion of LK03-FIX, and NHPs 3, 6, and 9 were selected for infusion of KP1-FIX, where FIX was the modified Factor IX (R318Y/R338E/T343R) codon optimized for mouse and containing the partial FIX intron, as detailed above. rAAV was prepared in a liquid formulation for intravenous infusion. AAV-LK03-FIX was a liquid formulation at 2.15 E+12 vg/mL. AAV-KP1-FIX was a liquid formulation at 1.17 E+12 vg/mL.

Particulars of the experimental groups, including dosage, AAV concentration, volume, and administration route are set forth in Table 59, below:

TABLE 59
Experimental Setup

| | | | Concen- | | | |
|---|---|---|---|---|---|---|
| | | | tration | Volume | | |
| Group | Treatment | Dose | (vg/ml) | (ml/kg) | Route | N |
| 1 | AAV-LK03 | 1.0e12 vg/kg | 2.16e12 | 0.46 | IV | 3 |
| | CB2679d | | | | | |
| 2 | AAV-KP1 | 1.0e12 vg/kg | 1.17e12 | 0.85 | | 3 |
| | CB2679d | | | | | |

Animals were anesthetized with intramuscular (IM) ketamine HCl (10 mg/kg) and isoflurane prior to all blood collections and intravenous (IV) dosing. rAAV (LK03 or KP1)-FIX was delivered to anesthetized animals through an IV catheter placed into the saphenous or cephalic vein, per laboratory Standard Operating Procedures (SOPs) for Procedures for Injections and Blood Withdrawal for Nonhuman Primates. AAV-LK03 (at 2.16e12 vg/mL) was dosed at 1.0e12 vg/kg NHP body weight (0.46 mL/kg), based on body weights obtained within one week of dosing. AAV-KP1 (1.17e12 vg/mL) was dosed at 1.0e12 vg/kg NHP body weight (0.85 mL/kg). rAAV-FIX was delivered over approximately 15 seconds, and then flushed with approximately 2 mL sterile saline.

Blood was collected for assessment of AAV neutralization, clinical chemistry, hematological assessments, coagulation, FIX expression, and FIX activity at 7 days prior to AAV infusion, and at days 3, 7, 14, 28, 42, 56 and 84 post-AAV infusion. Blood was collected from the femoral, saphenous, or cephalic veins of sedated animals.

C. AAV Neutralization in Selected NHPs

Animals producing the lowest levels of AAV-neutralizing antibodies from the experiments in section A, above, were selected for further analysis during the 12 week time course of the experiment. NHP animal numbers 3, 6 and 9, the animals injected with KP1-AAV vectors, and animal numbers 1, 5 and 7, the animals injected with LK03-AAV vectors, were further characterized.

1. Control Experiments Assessing LK03 And KP1 In Vitro Transduction

Prior to injection of the LK03-FIX and KP1-FIX AAV vectors into the selected NHP animals (NHP numbers 1, 3, 5, 6, 7, 9), transduction efficiencies of the LK03 and KP1 vector preparations were tested again in SNU-387 Hepatocellular carcinoma cells, as detailed above and essentially in accord with the protocol set forth in Meliani et al. ((2015) Human Gene Therapy Methods 26:45-53). Twenty-four hours after transduction, luciferase signal in the cells, which is indicative of transduction efficiency, was measured. The results show that luciferase signal increased with increasing multiplicity of infection (MOI). The signals for LK03-AAV and KP1-AAV were not significantly different; both vectors efficiently infected SNU-387 cells in these control experiments. The results are set forth as FLuc molecules (log₁₀) in Table 60, below:

TABLE 60
Transduction Efficiency of FLuc rAAV vectors
LK03-FIX and KP1-FIX on SNU-387 Cells

| | LK03 | KP1 |
|---|---|---|
| MOI 200 | 8.368 | 8.89 |

Next, pooled human intravenous immunoglobulin (IVIG) was used as a control/standard for neutralization of the AAVs LK03 and KP1. AAVs LK03-Luc and KP1-Luc were subject to serial dilution of the IVIG standard control, as detailed in section A, above, to assess antibody neutralization of the AAV. Results are set forth in Table 61, where data are presented as percent luciferase expression normalized to luciferase expression in the absence of IVIG (set to 100, no neutralization). These results replicate the initial experiments set forth above and show that LK03 and KP1 are neutralized with increasing concentrations of IVIG, as demonstrated by decreasing luciferase expression with increasing IVIG. LK03 is more susceptible to IVIG neutralization of relative total expression at all concentrations greater than 5 ug/mL, compared to KP 1.

TABLE 61
AAV Neutralization by Pooled Human IVIG

| IVIG (μg/mL) | LK03 | KP1 |
|---|---|---|
| 0 | 100 | 100 |
| 5 | 94.37 | 92.205 |
| 10 | 72.24 | 88.54 |
| 20 | 32.03 | 77.81 |
| 40 | 7.89 | 59.72 |
| 60 | 2.05 | 46.08 |
| 80 | 1.26 | 33.8 |
| 100 | 0.88 | 25.69 |
| 125 | 0.66 | 16.94 |
| 150 | 0.52 | 9.97 |
| 200 | 0.40 | 3.96 |
| 300 | 0.36 | 1.33 |

* data represented as percent luciferase expression normalized to luciferase expression in the absence of IVIG (set to 100, no neutralization)

2. KP1 and LK03 AAV In Vitro Transduction in the Presence of NHP Serum Isolated from Animals Weekly, for Eight Weeks

Next, the presence of AAV neutralizing antibodies during the 8 weeks post-AAV infusion was assessed in NHPs. Twenty-one days prior to AAV injection, blood was collected from the 6 female Cynomolgus monkeys selected above (NHP numbers 1, 3, 5, 6, 7, 9). The blood was processed into serum, and serum was heat-inactivated for 30 minutes at 56° C. Monkey serum then was serially diluted in the heat-inactivated FBS (undiluted, 1:3.16, 1:10, 1:31.6, 1:100, 1:316, 1:1000, 1:3160, 1:10000, 1:31600, 1:100000) or in medium with no serum. Next, 30 μL (1.4 E+7 vg) of the AAV-luciferase vector (LK03 or KP1) was incubated with 30 μL of undiluted serum, or a serum dilution (1:3.16, 1:10, 1:31.6, 1:100, 1:316, 1:1000, 1:3160, 1:10000, 1:31600, 1:100000) or no monkey serum as a negative control (FBS only) for 1 hour at 37° C. Following incubation, 22.5 μL of each serum-virus mixture was used to transduce SNU-387 cells in duplicates at a MOI of 200. SNU-387 cells had been seeded the day before at 2.5e4 cells/well in a 48 well plate.

After 24 hours of transfection, cells were rinsed with PBS and 100 μL of Passive Lysis buffer (Promega, Madison, Wis.; Cat. No. E194I) was added to each well. Next, cell lysates were collected, frozen, thawed, and centrifuged. After centrifugation, 20 μL of each clarified supernatant was extracted from the cell lysates and incubated with the luciferase substrate (Promega, Madison, Wis.; Cat. No. E1910) according to manufacturer's instructions, and luminescence was measured on a plate reader.

Two technical replicates of each sample dilution (or undiluted, or no serum control) were analyzed for luciferase luminescence. Data are expressed, in Tables 62-67, as percent luciferase expression normalized to luciferase expression in the absence of serum (set to 100, no neutralization).

When neutralizing antibodies (nAB) are present, low luciferase signal is detected in undiluted serum. Luciferase signal in serum collected from animals 21 days prior to AAV administration was approximately the same as the no serum control for four animals, two administered LK03 (NHP 5 and 7) and two administered KP1 (NHP 3 and 9). This indicates there were no neutralizing antibodies present in these animals prior to AAV administration. Conversely, NHP 6 (administered KP1) and NHP 1 (administered LK03) showed low luciferase in undiluted serum prior to AAV treatment, indicating the presence of neutralizing antibodies prior to AAV treatment. Following AAV administration, in all animals, luciferase signal was low in undiluted serum at all timepoints from one to seven weeks post-injection, indicating the presence of neutralizing antibodies. Luciferase expression in serially diluted serum elucidated variability in neutralizing antibodies levels among the different NHPs. In all animals administered LK03 (e.g., NHP 1, 5 and 7), luciferase signal decreased slightly one week after LK03-AAV injection in serum diluted 1:3160 and greater, increasing after two weeks to a consistent level that was similar to the pre-treatment values, and was maintained over seven weeks. Animals administered KP1-AAV showed generally higher neutralizing antibody levels than animals administered LK03, and KP1-NHP 3 showed low luciferase expression even at high dilutions, indicating high levels of neutralizing antibodies in this animal.

Data are set forth in Tables 62-67, below, as a percentage of the no serum control at 21 days prior to treatment (which is set to 100):

TABLE 62
Antibody Neutralization of AAV-KP1 NHP Animal No. 3

| | NHP Animal No. 3 | | | | | | |
|---|---|---|---|---|---|---|---|
| Day | −21 | 7 | 14 | 28 | 42 | 56 | 84 |
| No serum | 100 ± | 99.49 ± | 99.55 ± | 104.8 ± | 95.2 ± | 101.60 ± | 98.41 ± |
| | 3.82 | 1.78 | 0.62 | 4.6 | 5.78 | 4.21 | 2.23 |
| 1:100000 | 101.9 ± | 99.67 ± | 90.12 ± | 110.87 ± | 92.96 ± | 91.34 ± | 89.0 ± |
| | 10.1 | 2.36 | 2.49 | 4.1 | 8.09 | 4.58 | 5.09 |
| 1:31600 | 104.19 ± | 97.9 ± | 87.67 ± | 105.64 ± | 86.0 ± | 80.89 ± | 67.78 ± |
| | 6.17 | 1.79 | 12.43 | 9.93 | 6.62 | 3.31 | 5.81 |
| 1:10000 | 97.95 ± | 89.41 ± | 76.85 ± | 88.86 ± | 58.02 ± | 45.68 ± | 33.88 ± |
| | 4.46 | 5.58 | 5.23 | 6.78 | 3.19 | 3.21 | 5.81 |
| 1:3160 | 98.79 ± | 67.49 ± | 53.44 ± | 41.74 ± | 13.39 ± | 5.95 ± | 3.26 ± |
| | 3.38 | 4.83 | 5.0 | 2.95 | 1.18 | 1.52 | 0.08 |
| 1:1000 | 104.15 ± | 34.39 ± | 12.97 ± | 5.00 ± | 1.71 ± | 1.11 ± | 0.80 ± |
| | 10.99 | 1.86 | 8.56 | 0.21 | 0.15 | 0.05 | 0.04 |
| 1:316 | 102.83 ± | 29.94 ± | 2.11 ± | 5.16 ± | 0.73 ± | 2.14 ± | 0.43 ± |
| | 5.77 | 10.70 | 0.18 | 0.48 | 0.08 | 1.29 | 0.02 |
| 1:100 | 103.25 ± | 23.16 ± | 0.94 ± | 5.31 ± | 0.42 ± | 2.56 ± | 0.28 ± |
| | 2.27 | 2.73 | 0.14 | 0.53 | 0.02 | 2.51 | 0.02 |
| 1:31.6 | 108.13 ± | 27.33 ± | 0.6 ± | 5.41 ± | 0.26 ± | 1.42 ± | 0.20 ± |
| | 1.26 | 9.84 | 0.11 | 1.14 | 0.02 | 1.48 | 0.03 |
| 1:10 | 104.73 ± | 6.5 ± | 0.45 ± | 2.22 ± | 0.18 ± | 2.04 ± | 0.13 ± |
| | 8.67 | 5.42 | 0.11 | 2.62 | 0.01 | 2.22 | 0.00 |
| 1:3.16 | 109.8 ± | 1.26 ± | 0.33 ± | 0.32 ± | 0.13 ± | 0.16 ± | 0.11 ± |
| | 4.0 | 0.24 | 0.6 | 0.25 | 0.0 | 0.09 | 0.01 |
| Undil. | 117.8 ± | 0.67 ± | 0.29 ± | 0.14 ± | 0.11 ± | 0.10 ± | 0.08 ± |
| | 4.06 | 0.06 | 0.20 | 0.01 | 0.01 | 0.01 | 0.01 |

TABLE 63
Antibody Neutralization of AAV-KP1 NHP Animal No. 6

| | NHP Animal No. 6 | | | | | | |
|---|---|---|---|---|---|---|---|
| Day | −25 | 7 | 14 | 28 | 42 | 56 | 84 |
| No serum | 100 ± | 98.74 ± | 101.90 ± | 103.69 ± | 94.47 ± | 100.90 ± | 98.65 ± |
| | 4.92 | 0.22 | 0.40 | 10.21 | 2.03 | 6.48 | 5.08 |
| 1:100000 | 98.48 ± | 104.17 ± | 100.22 ± | 104.85 ± | 101.74 ± | 98.95 ± | 97.86 ± |
| | 2.23 | 1.11 | 3.78 | 9.53 | 6.10 | 3.33 | 3.48 |
| 1:31600 | 99.22 ± | 107.27 ± | 103.01 ± | 112.82 ± | 100.58 ± | 100.90 ± | 89.92 ± |
| | 3.61 | 14.48 | 15.88 | 12.56 | 3.34 | 1.75 | 3.63 |
| 1:10000 | 93.73 ± | 84.73 ± | 84.25 ± | 91.97 ± | 95.56 ± | 101.03 ± | 91.46 ± |
| | 3.26 | 0.42 | 4.20 | 1.46 | 8.36 | 7.82 | 12.21 |
| 1:3160 | 89.01 ± | 47.86 ± | 50.80 ± | 72.73 ± | 73.15 ± | 76.52 ± | 74.24 ± |
| | 11.24 | 0.79 | 3.95 | 2.54 | 2.16 | 1.29 | 3.73 |
| 1:1000 | 92.62 ± | 7.07 ± | 7.24 ± | 23.34 ± | 33.87 ± | 43.44 ± | 31.87 ± |
| | 9.58 | 0.90 | 0.56 | 0.36 | 1.85 | 3.91 | 5.63 |
| 1:316 | 97.12 ± | 1.53 ± | 1.42 ± | 2.98 ± | 4.13 ± | 5.41 ± | 2.98 ± |
| | 4.62 | 0.22 | 0.14 | 0.22 | 0.56 | 0.93 | 0.08 |
| 1:100 | 95.60 ± | 1.16 ± | 0.61 ± | 1.39 ± | 0.95 ± | 1.37 ± | 0.95 ± |
| | 5.25 | 0.55 | 0.04 | 0.37 | 0.04 | 0.34 | 0.08 |
| 1:31.6 | 72.66 ± | 1.47 ± | 0.35 ± | 1.53 ± | 0.53 ± | 0.96 ± | 0.50 ± |
| | 2.43 | 1.64 | 0.03 | 1.44 | 0.03 | 0.55 | 0.05 |
| 1:10 | 33.29 ± | 0.48 ± | 0.23 ± | 0.48 ± | 0.33 ± | 0.41 ± | 0.32 ± |
| | 1.18 | 0.25 | 0.00 | 0.12 | 0.04 | 0.07 | 0.02 |
| 1:3.16 | 4.88 ± | 0.19 ± | 0.18 ± | 0.25 ± | 0.25 ± | 0.26 ± | 0.23 ± |
| | 0.32 | 0.01 | 0.02 | 0.01 | 0.01 | 0.04 | 0.01 |
| Undil. | 1.33 ± | 0.13 ± | 0.13 ± | 0.21 ± | 0.20 ± | 0.19 ± | 0.18 ± |
| | 0.08 | 0.03 | 0.02 | 0.01 | 0.02 | 0.01 | 0.00 |

TABLE 64
Antibody Neutralization of AAV-KP1 NHP Animal No. 9

| | NHP Animal No. 9 | | | | | | |
|---|---|---|---|---|---|---|---|
| Day | −25 | 7 | 14 | 28 | 42 | 56 | 84 |
| No serum | 100.00 ± | 104.79 ± | 95.21 ± | 98.62 ± | 100.57 ± | 101.41 ± | 98.59 ± |
| | 9.47 | 6.75 | 3.68 | 4.19 | 6.81 | 5.97 | 11.81 |
| 1:100000 | 104.10 ± | 119.40 ± | 97.65 ± | 93.71 ± | 89.38 ± | 109.47 ± | 99.52 ± |
| | 10.38 | 2.73 | 7.16 | 11.63 | 5.35 | 11.14 | 6.20 |
| 1:31600 | 96.00 ± | 114.79 ± | 99.02 ± | 96.52 ± | 98.24 ± | 118.47 ± | 99.08 ± |
| | 5.82 | 7.47 | 3.25 | 8.59 | 10.11 | 12.62 | 4.38 |
| 1:10000 | 98.68 ± | 120.54 ± | 98.14 ± | 94.75 ± | 81.59 ± | 99.04 ± | 90.76 ± |
| | 10.10 | 11.15 | 8.06 | 10.23 | 8.04 | 5.69 | 3.13 |
| 1:3160 | 99.05 ± | 112.79 ± | 102.95 ± | 88.53 ± | 70.36 ± | 63.74 ± | 64.94 ± |
| | 6.23 | 6.88 | 2.11 | 14.30 | 4.21 | 4.13 | 0.49 |
| 1:1000 | 96.10 ± | 112.60 ± | 96.90 ± | 72.37 ± | 31.45 ± | 13.17 ± | 19.11 ± |
| | 5.95 | 1.96 | 3.96 | 5.32 | 1.09 | 8.09 | 1.21 |
| 1:316 | 100.92 ± | 94.01 ± | 85.47 ± | 35.25 ± | 4.63 ± | 2.55 ± | 2.03 ± |
| | 2.62 | 6.61 | 4.77 | 3.27 | 0.27 | 0.45 | 0.17 |
| 1:100 | 101.99 ± | 44.72 ± | 49.66 ± | 4.80 ± | 1.01 ± | 1.81 ± | 0.75 ± |
| | 4.41 | 2.22 | 1.90 | 1.45 | 0.03 | 0.99 | 0.08 |
| 1:31.6 | 100.03 ± | 19.36 ± | 14.67 ± | 6.14 ± | 0.52 ± | 4.46 ± | 0.44 ± |
| | 5.92 | 2.13 | 1.03 | 8.01 | 0.03 | 6.21 | 0.07 |
| 1:10 | 101.90 ± | 4.69 ± | 1.98 ± | 1.42 ± | 0.34 ± | 0.94 ± | 0.29 ± |
| | 5.19 | 2.84 | 0.15 | 1.05 | 0.03 | 0.75 | 0.04 |
| 1:3.16 | 105.98 ± | 0.87 ± | 0.83 ± | 0.31 ± | 0.25 ± | 0.23 ± | 0.21 ± |
| | 4.36 | 0.12 | 0.06 | 0.32 | 0.02 | 0.03 | 0.02 |
| Undil. | 109.07 ± | 0.57 ± | 0.54 ± | 0.27 ± | 0.21 ± | 0.14 ± | 0.16 ± |
| | 5.87 | 0.04 | 0.03 | 0.03 | 0.03 | 0.01 | 0.02 |

TABLE 65
Antibody Neutralization of AAV-LK03 NHP Animal No. 1

| | NHP Animal No. 1 | | | | | | |
|---|---|---|---|---|---|---|---|
| Day | −25 | 7 | 14 | 28 | 42 | 56 | 84 |
| No serum | 100 ± | 92.62 ± | 107.38 ± | 98.29 ± | 101.72 ± | 98.31 ± | 101.69 ± |
| | 3.95 | 1.97 | 3.21 | 6.36 | 0.33 | 1.50 | 1.12 |
| 1:100000 | 114.79 ± | 112.02 ± | 124.92 ± | 116.55 ± | 119.34 ± | 108.99 ± | 120.02 ± |
| | 4.96 | 21.16 | 10.56 | 5.62 | 8.47 | 15.40 | 1.45 |
| 1:31600 | 121.21 ± | 107.66 ± | 125.05 ± | 119.15 ± | 126.18 ± | 111.48 ± | 119.76 ± |
| | 12.50 | 7.96 | 6.42 | 3.55 | 3.67 | 9.70 | 6.19 |
| 1:10000 | 113.32 ± | 96.70 ± | 124.15 ± | 117.27 ± | 123.05 ± | 122.00 ± | 116.23 ± |
| | 3.44 | 12.96 | 0.44 | 5.95 | 7.16 | 11.38 | 5.90 |
| 1:3160 | 117.76 ± | 96.50 ± | 116.94 ± | 128.06 ± | 107.56 ± | 122.22 ± | 117.90 ± |
| | 10.46 | 16.93 | 7.24 | 3.01 | 5.22 | 12.85 | 15.26 |
| 1:1000 | 122.83 ± | 48.35 ± | 80.89 ± | 108.93 ± | 114.28 ± | 112.45 ± | 107.92 ± |
| | 13.04 | 7.43 | 4.38 | 7.09 | 13.41 | 10.40 | 8.46 |
| 1:316 | 120.93 ± | 7.61 ± | 25.35 ± | 68.33 ± | 83.44 ± | 92.00 ± | 89.46 ± |
| | 11.08 | 0.98 | 2.23 | 2.59 | 0.37 | 5.55 | 11.19 |
| 1:100 | 113.07 ± | 1.21 ± | 2.21 ± | 14.96 ± | 34.09 ± | 40.23 ± | 48.07 ± |
| | 12.41 | 0.10 | 0.19 | 1.50 | 1.69 | 4.81 | 0.96 |
| 1:31.6 | 98.97 ± | 0.65 ± | 0.72 ± | 2.01 ± | 3.16 ± | 4.25 ± | 6.00 ± |
| | 18.57 | 0.08 | 0.04 | 0.04 | 0.58 | 0.38 | 0.26 |
| 1:10 | 64.45 ± | 0.39 ± | 0.43 ± | 0.92 ± | 0.87 ± | 1.21 ± | 1.19 ± |
| | 13.60 | 0.02 | 0.02 | 0.04 | 0.08 | 0.08 | 0.04 |
| 1:3.16 | 15.19 ± | 0.28 ± | 0.27 ± | 0.54 ± | 0.52 ± | 0.71 ± | 0.66 ± |
| | 5.30 | 0.02 | 0.02 | 0.05 | 0.06 | 0.04 | 0.02 |
| Undil. | 1.77 ± | 0.22 ± | 0.19 ± | 0.48 ± | 0.43 ± | 0.52 ± | 0.48 ± |
| | 0.06 | 0.02 | 0.01 | 0.02 | 0.06 | 0.04 | 0.02 |

TABLE 66
Antibody Neutralization of AAV-LK03 NHP Animal No. 5

| | NHP Animal No. 5 | | | | | | |
|---|---|---|---|---|---|---|---|
| Day | −25 | 7 | 14 | 28 | 42 | 56 | 84 |
| No serum | 100 ± | 95.03 ± | 104.98 ± | 95.74 ± | 104.27 ± | 99.02 ± | 100.99 ± |
| | 5.01 | 7.39 | 1.42 | 5.75 | 8.77 | 2.43 | 1.22 |
| 1:100000 | 133.40 ± | 115.37 ± | 129.14 ± | 112.24 ± | 116.36 ± | 120.78 ± | 139.91 ± |
| | 5.34 | 19.17 | 10.17 | 11.15 | 17.11 | 9.92 | 11.35 |
| 1:31600 | 133.05 ± | 121.70 ± | 124.16 ± | 103.97 ± | 110.48 ± | 119.20 ± | 132.19 ± |
| | 7.73 | 14.01 | 8.11 | 2.28 | 12.81 | 9.03 | 20.65 |
| 1:10000 | 126.94 ± | 109.71 ± | 116.83 ± | 95.16 ± | 91.47 ± | 111.61 ± | 124.80 ± |
| | 6.50 | 7.35 | 8.16 | 9.28 | 9.56 | 8.91 | 11.92 |
| 1:3160 | 118.97 ± | 109.02 ± | 86.11 ± | 46.62 ± | 58.15 ± | 70.28 ± | 93.04 ± |
| | 2.17 | 10.30 | 5.09 | 10.04 | 6.98 | 13.14 | 1.79 |
| 1:1000 | 119.95 ± | 89.75 ± | 38.02 ± | 8.5 ± | 9.30 ± | 18.12 ± | 43.78 ± |
| | 16.21 | 8.06 | 1.52 | 1.01 | 1.49 | 4.13 | 2.40 |
| 1:316 | 123.45 ± | 39.89 ± | 5.11 ± | 1.26 ± | 1.21 ± | 1.53 ± | 4.39 ± |
| | 5.35 | 2.14 | 0.32 | 0.09 | 0.05 | 0.21 | 0.65 |
| 1:100 | 127.31 ± | 6.82 ± | 1.26 ± | 0.64 ± | 0.58 ± | 0.79 ± | 1.23 ± |
| | 11.72 | 1.25 | 0.02 | 0.05 | 0.04 | 0.04 | 0.02 |
| 1:31.6 | 128.06 ± | 1.32 ± | 0.59 ± | 0.36 ± | 0.31 ± | 0.49 ± | 0.63 ± |
| | 6.39 | 0.16 | 0.04 | 0.02 | 0.02 | 0.05 | 0.10 |
| 1:10 | 126.94 ± | 0.66 ± | 0.49 ± | 0.26 ± | 0.26 ± | 0.36 ± | 0.43 ± |
| | 13.04 | 0.03 | 0.02 | 0.04 | 0.02 | 0.02 | 0.03 |
| 1:3.16 | 129.12 ± | 0.45 ± | 0.3 ± | 0.18 ± | 0.20 ± | 0.28 ± | 0.29 ± |
| | 13.36 | 0.04 | 0.03 | 0.02 | 0.04 | 0.01 | 0.05 |
| Undil. | 129.24 ± | 0.32 ± | 0.25 ± | 0.16 ± | 0.14 ± | 0.22 ± | 0.19 ± |
| | 11.05 | 0.02 | 0.04 | 0.01 | 0.02 | 0.02 | 0.02 |

TABLE 67
Antibody Neutralization of AAV-LK03 NHP Animal No. 7

| | NHP Animal No. 7 | | | | | | |
|---|---|---|---|---|---|---|---|
| Day | −25 | 7 | 14 | 28 | 42 | 56 | 84 |
| No serum | 100 ± | 95.92 ± | 104.09 ± | 101.13 ± | 98.88 ± | 102.22 ± | 97.78 ± |
| | 0.78 | 8.03 | 4.12 | 2.48 | 5.34 | 3.44 | 13.73 |
| 1:100000 | 119.02 ± | 109.83 ± | 96.78 ± | 113.121 ± | 109.17 ± | 113.31 ± | 121.92 ± |
| | 7.44 | 6.48 | 8.50 | 17.42 | 8.45 | 6.72 | 16.14 |
| 1:31600 | 123.43 ± | 111.08 ± | 102.49 ± | 105.52 ± | 110.11 ± | 118.80 ± | 122.97 ± |
| | 8.71 | 8.51 | 10.74 | 8.38 | 11.67 | 13.98 | 15.28 |
| 1:10000 | 128.09 ± | 110.09 ± | 100.05 ± | 107.56 ± | 111.04 ± | 119.69 ± | 116.47 ± |
| | 5.12 | 2.57 | 14.33 | 12.16 | 10.80 | 6.18 | 12.93 |
| 1:3160 | 132.35 ± | 92.29 ± | 98.07 ± | 105.47 ± | 116.96 ± | 121.87 ± | 124.78 ± |
| | 11.75 | 0.92 | 13.14 | 6.31 | 6.29 | 12.15 | 9.40 |
| 1:1000 | 123.16 ± | 68.99 ± | 86.95 ± | 99.29 ± | 115.37 ± | 112.82 ± | 121.66 ± |
| | 4.32 | 2.89 | 7.95 | 2.23 | 3.03 | 15.13 | 9.37 |
| 1:316 | 121.76 ± | 27.87 ± | 67.01 ± | 92.11 ± | 98.51 ± | 101.95 ± | 110.25 ± |
| | 5.89 | 0.78 | 6.74 | 1.10 | 7.52 | 6.03 | 1.34 |
| 1:100 | 125.07 ± | 3.41 ± | 21.09 ± | 67.46 ± | 71.05 ± | 75.46 ± | 82.17 ± |
| | 4.79 | 0.35 | 4.21 | 2.40 | 6.49 | 10.08 | 7.51 |
| 1:31.6 | 130.03 ± | 0.99 ± | 2.04 ± | 16.79 ± | 23.80 ± | 20.82 ± | 26.85 ± |
| | 2.36 | 0.09 | 0.61 | 3.47 | 2.24 | 4.64 | 1.49 |
| 1:10 | 124.54 ± | 0.52 ± | 0.65 ± | 2.06 ± | 2.25 ± | 2.38 ± | 2.94 ± |
| | 6.51 | 0.03 | 0.02 | 0.29 | 0.15 | 0.16 | 0.17 |
| 1:3.16 | 141.34 ± | 0.37 ± | 0.41 ± | 0.82 ± | 0.90 ± | 1.03 ± | 1.07 ± |
| | 3.23 | 0.04 | 0.01 | 0.03 | 0.11 | 0.05 | 0.10 |
| Undil. | 133.47 ± | 0.26 ± | 0.27 ± | 0.60 ± | 0.59 ± | 0.68 ± | 0.78 ± |
| | 11.77 | 0.02 | 0.04 | 0.07 | 0.02 | 0.07 | 0.06 |

3. Anti-Drug Antibodies

Assays for anti-drug antibodies (ADAs) were conducted to aid in assessing any immune response to modified FIX in NHPs administered AAV-KP1-FIX and AAV-LK03-FIX. Citrated plasma was prepared from blood, and FIX concentration in plasma was determined using a bridging electro-chemiluminescent immunoassay (ECLIA) using Meso Scale Discovery (MSD) technology. ECLIA is similar to ELISA, but uses an electrochemiluminesent signal rather than a colorimetric reaction. Biotin-conjugated modified (R318Y/R338E/T343R) FIX at 500 ng/mL was added to a streptavidin-coated assay plate. Plasma samples were diluted in acetic acid solution (AAWS) to a minimum required dilution of 1/10, and then added to the plate. Ruthenium-conjugated (sulfo-tagged) modified (R318Y/R338E/T343R) FIX, which produces light upon application of an electrical potential, was then added at 500 ng/mL. Any drug-antibody-drug complexes remaining after the plate was washed were detected in relative light units (RLU) on an MSD SECTOR™ Imager 600 reader (Meso Scale Diagnostics, LLC. Rockville, Md.). A positive signal above background reflect the presence of anti-FIX antibodies in the sample.

In this assay, the negative control is 1/10 diluted pooled monkey plasma in AAWS. The plate specific cut point (PSCP) was the sum of the RLU of the negative control RLU and the pre-defined screening cut-point factor (here 9 RLU), which was defined during method validation. Samples were considered positive if their mean RLU was greater than or equal to the PSCP.

Samples spiked with a 25 μg/mL solution of modified FIX (R318Y/R338E/T343R) in AAWS or those substituted with an equivalent volume of AAWS prior to addition to the MSD plate served as additional controls. Added modified FIX (R318Y/R338E/T343R) competes with the ability of sample ADAs to bind the capture and the detection of the modified FIX. A positive sample was identified as a spiked sample with a minimum percent inhibition of RLU signal of 15.6% compared to its unspiked pair, where the unspiked sample signal was one that remained above the PSCP.

The results show two animals administered LK03 (NHP 5 and 1) and two administered KP1 (NHP 3 and 9) showed high chemiluminescence signal, indicating high levels of anti-FIX antibodies. Positive signals were first detected at study day 28 for both LK03-FIX and KP1-FIX and generally increased in signal intensity over time. NHP 7, which was administered LK03-FIX, and NHP 6, which was administered KP1-FIX, did not show an increase of chemiluminescence over time, indicating that anti-FIX antibodies did not develop over the 13 week course of the experiment. The results are set forth in Table 68, below:

TABLE 68
Electrochemiluminesent signal results from ADA assay

| | Day | | | | | | | | | | |
|---|---|---|---|---|---|---|---|---|---|---|---|
| NHP No. | −7 | 0 | 3 | 7 | 14 | 28 | 42 | 56 | 84 | 98 | AAV |
| 5 | 72 | 68 | 73 | 74 | 77 | 100 | 1722 | 8550 | 18061 | 15195 | LK03 |
| 7 | 76 | 68 | 74 | 72 | 73 | 73 | 82 | 78 | 100 | 92 | |
| 1 | 76 | 78 | 75 | 78 | 92 | 89 | 97 | 146 | 7871 | 16199 | |
| 3 | 62 | 61 | 61 | 71 | 74 | 74 | 74 | 87 | 1796 | 8546 | KP1 |
| 6 | 68 | 68 | 71 | 68 | 76 | 64 | 67 | 62 | 74 | 78 | |
| 9 | 71 | 70 | 70 | 69 | 81 | 2254 | 15306 | 21593 | n/d | 9876 | |

*NHP animals 1, 3, 5, and 9 had positive confirmatory testing results for ADA presence (cells highlighted in bold)

4. Clinical Chemistry

For clinical chemistry analyses, approximately 1 mL whole blood was collected from each animal 7 days prior to AAV infusion, the day of infusion (day 0), and at days 3, 7, 14, 28, 42, 56 and 84 post-infusion. Blood samples were allowed to clot at room temperature for at least 15 minutes and up to two hours prior to centrifugation and then loaded into a serum separator or clot tube and centrifuged at 2500×g for 10 minutes at room temperature to separate into cellular and serum fractions. Samples were analyzed on a Roche/Hitachi Cobas Clinical Chemistry System (Roche Diagnostics, Indianapolis, Ind.) per laboratory SOP (Routine Operation of the Roche/Hitachi cobas c501 Chemistry Analyzer) in accord with manufacturer's instructions.

The serum chemistry results show that animals were similarly unaffected by administration of rAAV-LK03 and rAAV-KP1; serum chemistry remained stable before and after AAV administration and were within normal ranges for NHPs, with a few exceptions. Alanine Aminotransferase (Alanine Transaminase) levels were higher in animals administered KP1 compared to LK03; notably, these animals displayed higher average baseline levels of these enzymes prior to administration of KP1 compared to animals administered LK03. This indicates that the higher levels are not entirely be due to AAV administration. In another example, Gamma-Glutamyltransferase levels were higher in a single animal that had pre-existing nAbs. In another example, Alkaline Phosphatase levels were higher in animals administered LK03 compared to KP1; these animals also displayed higher average baseline levels of the enzyme prior to administration of LK03 compared to animals administered KP1 indicating that the higher levels are not entirely due to AAV administration. A selected group of serum chemistry parameters (Alanine Aminotransferase (Alanine Transaminase), Aspartate Aminotransferase (Aspartate Transaminase), and Gamma Glutamyltransferase) were analyzed relative to levels at 7 days prior to AAV infusion. The results of these normalized results show a steady level of these enzymes with rAAV-LK03 and rAAV-KP1, with a slight increase in Alanine Aminotransferase (Alanine Transaminase) with both AAVs.

Individual animal results for a subset of analyses are set forth in Table 71 and 73 and distinguish inter-animal variability within groups of animals administered KP1 or LK03. The results show that although there was some minor variability in serum chemistry results between animals, these differences did not appear significant indicating that KP1-AAV and LK03-AAV were well-tolerated.

The clinical chemistry parameters assessed are set forth in Table 69; the serum chemistry results, expressed as an average of the three animals for each group, are set forth in Table 70; and the select enzyme normalized to pretreatment with rAAVs are set forth in Table 72. Tables 69-74 are set forth below:

TABLE 69
Serum Chemistry Results

| Analyte | Abbreviation | Units |
|---|---|---|
| Alanine Aminotransferase | ALT | IU/L |
| (Alanine Transaminase) | | |
| Albumin (ALB) | ALB | g/dL |
| Alkaline Phosphatase (ALP) | ALP | IU/L |
| Aspartate Aminotransferase | AST | IU/L |
| (Aspartate Transaminase) | | |
| Bilirubin (Total) | BILI-T | mg/dL |
| Blood Urea Nitrogen | BUN | mg/dl |
| Chloride | CL-S | mmol/L |
| Creatinine | CRE-S | mg/dL |
| Glucose | GLU | mg/dL |
| Gamma Glutamyltransferase | GGT | IU/L |
| Lactate Dehydrogenase | LDH | IU/L |
| Phosphorous | | mg/dL |
| Potassium | K-S | mmol/L |
| Protein (Total) (TP) | TP | g/dL |
| Sodium | NA-S | mmol/L |
| Calculated Parameters and Ratios | | |
| Albumin/Globulin | A/G | None |
| Blood Urea Nitrogen/Creatinine | BUN/CRE | None |
| Globulin (GLOB) | GLOBN | g/dL |

TABLE 70
Serum Chemistry Results

| | Day | | | | | | | | | |
|---|---|---|---|---|---|---|---|---|---|---|
| Analyte | −7 | 0 | 3 | 7 | 14 | 28 | 42 | 56 | 84 | AAV |
| Alanine | 20.67 | 23.00 | 26.00 | 52.67 | 45.67 | 37.33 | 45.67 | 39.67 | 42.00 | LK03 |
| Aminotransferase | 40.67 | 46.67 | 38.00 | 97.00 | 86.33 | 63.00 | 65.33 | 81.67 | 78.00 | KP1 |
| (Alanine | | | | | | | | | | |
| Transaminase) | | | | | | | | | | |
| | 4.00 | 4.00 | 4.00 | 4.00 | 4.27 | 4.20 | 4.43 | 4.50 | 4.07 | LK03 |

TABLE 70
Serum Chemistry Results

| | Day | | | | | | | | | |
|---|---|---|---|---|---|---|---|---|---|---|
| | −7 | 0 | 3 | 7 | 14 | 28 | 42 | 56 | 84 | AAV |
| Analyte | | | | | | | | | | |
| Albumin (ALB) | 3.67 | 4.00 | 3.33 | 4.00 | 4.13 | 4.13 | 4.17 | 4.30 | 3.87 | KP1 |
| Alkaline | 231.67 | 186.00 | 189.00 | 219.67 | 210.33 | 207.67 | 252.00 | 242.33 | 226.67 | LK03 |
| Phosphatase (ALP) | 157.00 | 157.33 | 135.00 | 159.33 | 148.33 | 166.67 | 156.67 | 194.67 | 158.00 | KP1 |
| Aspartate | 43.67 | 52.00 | 45.33 | 54.33 | 45.00 | 45.67 | 58.00 | 59.33 | 50.00 | LK03 |
| Aminotransferase | 43.00 | 52.33 | 50.33 | 50.00 | 39.67 | 41.00 | 46.33 | 50.00 | 50.67 | KP1 |
| (Aspartate | | | | | | | | | | |
| Transaminase) | | | | | | | | | | |
| Bilirubin (Total) | 0.20 | 0.20 | 0.20 | 0.20 | 0.15 | 0.15 | 0.20 | 0.15 | 0.15 | LK03 |
| | 0.20 | 0.20 | 0.20 | 0.20 | 0.15 | 0.15 | 0.20 | 0.15 | 0.15 | KP1 |
| Blood Urea | 15.33 | 21.00 | 17.00 | 18.00 | 20.67 | 20.67 | 18.00 | 23.00 | 18.33 | LK03 |
| Nitrogen | 13.67 | 18.00 | 14.00 | 12.67 | 18.33 | 14.67 | 14.33 | 15.67 | 15.67 | KP1 |
| Chloride | 106.00 | 106.00 | 103.00 | 105.67 | 104.67 | 105.67 | 105.00 | 109.00 | 105.33 | LK03 |
| | 107.67 | 110.67 | 106.33 | 106.67 | 105.33 | 106.67 | 108.00 | 110.33 | 104.00 | KP1 |
| Creatinine | 0.67 | 1.00 | 0.67 | 1.00 | 0.60 | 0.57 | 0.60 | 0.67 | 0.63 | LK03 |
| | 1.00 | 1.00 | 1.00 | 1.00 | 0.73 | 0.67 | 0.70 | 0.83 | 0.73 | KP1 |
| Glucose | 75.67 | 53.33 | 55.33 | 65.00 | 74.67 | 67.00 | 52.00 | 72.00 | 67.00 | LK03 |
| | 71.00 | 59.67 | 55.00 | 65.67 | 77.67 | 75.00 | 55.00 | 80.00 | 76.00 | KP1 |
| Gamma | 56.00 | 52.33 | 47.67 | 57.00 | 57.00 | 56.00 | 59.00 | 62.00 | 58.67 | LK03 |
| Glutamyltransferase | 71.33 | 68.67 | 64.00 | 74.67 | 75.00 | 77.00 | 74.67 | 81.00 | 77.67 | KP1 |
| Lactate | 456.33 | 486.00 | 538.67 | 553.00 | 451.33 | 542.33 | 562.33 | nd | nd | LK03 |
| Dehydrogenase | 551.67 | 677.33 | 722.67 | 546.00 | 415.33 | 559.67 | 658.33 | nd | nd | KP1 |
| Phosphorus | 4.67 | 4.33 | 4.33 | 5.33 | 24.37 | 4.47 | 5.20 | 5.47 | 4.77 | LK03 |
| | 5.00 | 5.00 | 4.33 | 6.00 | 4.67 | 5.20 | 5.10 | 6.40 | 6.20 | KP1 |
| Potassium | 3.33 | 4.00 | 3.67 | 3.67 | 3.60 | nd | 3.60 | 3.80 | 3.50 | LK03 |
| | 3.67 | 3.67 | 3.67 | 3.67 | 3.60 | nd | 3.33 | 3.90 | 3.60 | KP1 |
| Protein (Total) (TP) | 6.33 | 6.33 | 6.00 | 7.00 | 7.20 | 6.87 | 7.40 | 7.83 | 7.10 | LK03 |
| | 6.33 | 7.00 | 6.00 | 7.00 | 7.03 | 6.90 | 7.07 | 7.60 | 6.87 | KP1 |
| Sodium | 146.67 | 149.33 | 142.00 | 146.67 | 145.33 | 146.33 | 147.33 | 155.33 | 144.67 | LK03 |
| | 149.00 | 152.00 | 144.33 | 147.00 | 145.67 | 147.67 | 149.00 | 156.33 | 145.67 | KP1 |
| Calculated Parameters | | | | | | | | | | |
| and Ratios | | | | | | | | | | |
| Albumin/Globulin | 1.67 | 1.33 | 1.67 | 1.67 | 1.47 | 1.60 | 1.50 | 1.37 | 1.37 | LK03 |
| | 1.67 | 1.67 | 1.33 | 1.67 | 1.47 | 1.53 | 1.50 | 1.30 | 1.30 | KP1 |
| Blood Urea | 30.00 | 39.33 | 37.00 | 30.33 | 34.67 | 37.33 | 30.67 | 34.33 | 29.33 | LK03 |
| Nitrogen/Creatinine | 22.00 | 32.33 | 27.33 | 20.00 | 26.67 | 22.67 | 22.00 | 19.67 | 21.33 | KP1 |
| Globulin (GLOB) | 2.33 | 3.00 | 2.33 | 3.00 | 2.93 | 2.67 | 2.97 | 3.33 | 3.03 | LK03 |
| | 2.33 | 3.00 | 2.67 | 2.67 | 2.90 | 2.77 | 2.90 | 3.30 | 3.00 | KP1 |

TABLE 71
Select Results for Individual Animals

| | Day | | | | | | | | | | |
|---|---|---|---|---|---|---|---|---|---|---|---|
| Analyte | NHP No. | −7 | 0 | 3 | 7 | 14 | 28 | 42 | 56 | 84 | AAV |
| Lactate | 5 | 387 | 459 | 400 | 412 | 414 | 462 | 595 | nd | nd | LK03 |
| Dehydrogenase | 7 | 232 | 385 | 324 | 387 | 295 | 441 | 486 | nd | nd | |
| | 1 | 750 | 614 | 892 | 860 | 645 | 724 | 606 | nd | nd | |
| | 3 | 436 | 468 | 332 | 400 | 363 | 396 | 421 | nd | nd | KP1 |
| | 6 | 619 | 643 | 668 | 568 | 449 | 533 | 620 | nd | nd | |
| | 9 | 600 | 921 | 1168 | 670 | 434 | 750 | 934 | nd | nd | |
| Globulin | 5 | 2.2 | 2.8 | 2.3 | 3.0 | 3.1 | 2.6 | 3.1 | 3.3 | 2.9 | LK03 |
| | 7 | 2.4 | 2.7 | 2.2 | 2.6 | 2.6 | 2.4 | 2.5 | 3.0 | 2.7 | |
| | 1 | 3.0 | 2.6 | 2.6 | 3.0 | 3.1 | 3.0 | 3.3 | 3.7 | 3.5 | |
| | 3 | 3.4 | 3.0 | 3.1 | 3.4 | 3.6 | 3.3 | 3.6 | 3.7 | 3.4 | KP1 |
| | 6 | 2.3 | 2.7 | 2.8 | 2.7 | 2.8 | 2.7 | 2.7 | 3.2 | 2.9 | |
| | 9 | 1.9 | 2.6 | 2.0 | 2.2 | 2.3 | 2.3 | 2.4 | 3.0 | 2.7 | |
| Alkaline | 5 | 85 | 87 | 80 | 96 | 88 | 84 | 104 | 101 | 86 | LK03 |
| Phosphatase | 7 | 287 | 233 | 239 | 280 | 277 | 285 | 366 | 366 | 331 | |
| (ALP) | 1 | 323 | 238 | 248 | 283 | 266 | 254 | 286 | 260 | 263 | |
| | 3 | 91 | 80 | 82 | 88 | 85 | 105 | 89 | 170 | 99 | KP1 |
| | 6 | 156 | 147 | 111 | 143 | 141 | 159 | 139 | 176 | 153 | |
| | 9 | 224 | 245 | 212 | 247 | 219 | 236 | 242 | 238 | 222 | |

TABLE 72
Normalized Serum Chemistry Results

| | Day | | | | | | | | | |
|---|---|---|---|---|---|---|---|---|---|---|
| Analyte | −7 | 0 | 3 | 7 | 14 | 28 | 42 | 56 | 84 | AAV |
| Alanine | 1.00 | 1.13 | 1.30 | 2.53 | 2.20 | 1.80 | 2.23 | 1.90 | 2.03 | LK03 |
| Aminotransferase | 1.00 | 1.13 | 0.90 | 2.37 | 2.10 | 1.57 | 1.60 | 2.03 | 1.90 | KP1 |
| (Alanine | | | | | | | | | | |
| Transaminase) | | | | | | | | | | |
| Aspartate | 1.00 | 1.23 | 1.07 | 1.23 | 1.03 | 1.07 | 1.33 | 1.37 | 1.13 | LK03 |
| Aminotransferase | 1.00 | 1.23 | 1.17 | 1.13 | 0.93 | 0.97 | 1.07 | 2.63 | 1.20 | KP1 |
| (Aspartate | | | | | | | | | | |
| Transaminase) | | | | | | | | | | |
| Gamma | 1.00 | 0.93 | 0.87 | 1.00 | 1.03 | 1.00 | 1.07 | 1.13 | 1.03 | LK03 |
| Glutamyltransferase | 1.00 | 0.93 | 0.90 | 1.07 | 1.03 | 1.10 | 1.07 | 1.10 | 1.10 | KP1 |

TABLE 73
Normalized Serum Chemistry Result for Individual Animals

| | Day | | | | | | | | | | |
|---|---|---|---|---|---|---|---|---|---|---|---|
| Analyte | NHP No. | −7 | 0 | 3 | 7 | 14 | 28 | 42 | 56 | 84 | AAV |
| Alanine | 5 | 21 | 29 | 26 | 50 | 50 | 38 | 51 | 44 | 47 | LK03 |
| Aminotransferase | 7 | 25 | 22 | 28 | 51 | 37 | 35 | 47 | 36 | 39 | |
| (Alanine | 1 | 16 | 18 | 24 | 56 | 50 | 39 | 39 | 39 | 40 | |
| Transaminase) | 3 | 71 | 79 | 30 | 90 | 83 | 104 | 105 | 137 | 142 | KP1 |
| | 6 | 44 | 44 | 67 | 175 | 151 | 60 | 66 | 72 | 58 | |
| | 9 | 7 | 17 | 17 | 26 | 25 | 25 | 25 | 36 | 34 | |
| | 5 | 40 | 46 | 48 | 50 | 46 | 45 | 61 | 61 | 50 | LK03 |
| | 7 | 44 | 51 | 42 | 56 | 44 | 46 | 70 | 58 | 47 | |
| Aspartate | 1 | 47 | 59 | 46 | 57 | 45 | 46 | 43 | 59 | 53 | |
| Aminotransferase | 3 | 48 | 42 | 40 | 45 | 39 | 45 | 43 | 85 | 41 | |
| (Aspartate | 6 | 42 | 47 | 56 | 56 | 47 | 41 | 49 | 98 | 44 | KP1 |
| Transaminase) | 9 | 39 | 68 | 55 | 49 | 33 | 37 | 47 | 154 | 67 | |
| Gamma | 5 | 45 | 52 | 44 | 53 | 55 | 51 | 55 | 61 | 55 | LK03 |
| Glutamyltransferase | 7 | 81 | 72 | 66 | 79 | 77 | 79 | 84 | 82 | 81 | |
| | 1 | 42 | 33 | 33 | 39 | 39 | 38 | 38 | 43 | 40 | |
| | 3 | 94 | 80 | 89 | 92 | 92 | 100 | 97 | 103 | 105 | KP1 |
| | 6 | 69 | 66 | 54 | 75 | 80 | 68 | 68 | 74 | 70 | LK03 |
| | 9 | 51 | 60 | 49 | 57 | 53 | 63 | 59 | 66 | 58 | |

5. Hematology Parameters

For hematological analyses, approximately 0.5 mL whole blood was collected from each animal at each of 7 days prior to AAV infusion, the day of infusion (day 0), and at days 3, 7, 14, 28, 42, 56 and 84 post-infusion, as detailed above, and placed into tubes containing ethylene diamine tetra acetate (K2EDTA) as an anticoagulant. Hematology samples were maintained at room temperature or refrigerated at 2-8° C. until analysis. Samples were analyzed on a Roche/Hitachi Cobas Clinical Chemistry System (Roche Diagnostics, Indianapolis, Ind.) per laboratory SOP (Routine Operation of the Roche/Hitachi cobas c501 Chemistry Analyzer) in accord with manufacturer's instructions.

The hematological analysis results show that animals were similarly unaffected by administration of rAAV-LK03 and rAAV-KP1; hematological markers remained stable before and after AAV administration and were within normal ranges for all NHPs. The observed transient decreases in red blood cells and hematocrit accompanied by transient increases in reticulocytes at the early timepoints can be attributed to a compensatory response due to more frequent blood collection. Baseline levels of total white blood cells (WBC) overall were higher in animals assigned to Group 1 (LK03) than in animals assigned to Group 2 (KP1), but treatment had no apparent effect on individual animal WBC counts, as the absolute and the relative concentrations remained relatively stable throughout the duration of the study.

The hematology parameters assessed are set forth in Table 74; and the hematology results, expressed as an average of the three animals for each group, are set forth in Table 75, each below:

TABLE 74
Hematology Parameters

| Parameter | Abbreviation^{A} | Units |
|---|---|---|
| Red Blood Cell Count | RBC | 10⁶/μL |
| Hemoglobin | HGB | g/dL |
| Hematocrit | HCT | % |
| Mean Corpuscular Volume | MCV | fL |
| Mean Corpuscular Hemoglobin | MCHC | g/dL |
| Concentration | | |
| Mean Corpuscular Hemoglobin | MCH | pg |
| Platelet Count | PLT | 10³/μL |
| Absolute Reticulocyte | AbRETIC | K/μL |
| Percent Reticulocytes | RETIC | % RBC |

White Blood Cell Count and Absolute and Percentage Differential

| | | |
|---|---|---|
| White Blood Cell Count | WBC | 10³/μL (and %) |
| Neutrophils | PMN | 10³/μL (and %) |
| Lymphocytes | LYM | 10³/μL (and %) |
| Monocytes | MONO | 10³/μL (and %) |
| Eosinophils | EOS | 10³/μL (and %) |
| Basophils | BASO | 10³/μL (and %) |

TABLE 75
Hematology Results

| | Day | | | | | | | | | |
|---|---|---|---|---|---|---|---|---|---|---|
| Parameter | −7 | 0 | 3 | 7 | 14 | 28 | 42 | 56 | 84 | AAV |
| Red Blood Cell | 6.29 | 5.41 | 4.70 | 4.95 | 5.34 | 5.51 | 6.00 | 5.78 | 5.85 | LK03 |
| Count | | | | | | | | | | |
| | 6.09 | 5.77 | 5.80 | 5.62 | 5.60 | 5.98 | 5.96 | 6.08 | 6.30 | KP1 |
| Hemoglobin | 14.70 | 12.23 | 10.67 | 11.27 | 12.20 | 12.50 | 13.60 | 13.00 | 13.30 | LK03 |
| | 13.47 | 12.70 | 12.83 | 12.40 | 12.40 | 13.17 | 13.20 | 13.37 | 13.73 | KP1 |
| Hematocrit | 46.47 | 40.73 | 35.40 | 37.80 | 40.73 | 42.23 | 47.20 | 43.17 | 43.70 | LK03 |
| | 44.93 | 42.77 | 42.70 | 41.37 | 42.13 | 45.03 | 46.23 | 44.90 | 45.97 | KP1 |
| Mean | 74.00 | 75.33 | 75.67 | 76.33 | 76.33 | 77.00 | 78.67 | 75.00 | 74.67 | LK03 |
| Corpuscular | | | | | | | | | | |
| Volume | | | | | | | | | | |
| | 73.67 | 74.33 | 73.67 | 73.67 | 75.00 | 75.00 | 77.67 | 74.00 | 73.00 | KP1 |
| Mean | 23.37 | 22.60 | 22.80 | 22.73 | 22.83 | 22.70 | 22.70 | 22.50 | 22.70 | LK03 |
| Corpuscular | | | | | | | | | | |
| Hemoglobin | | | | | | | | | | |
| Concentration | | | | | | | | | | |
| | 22.03 | 22.07 | 22.07 | 22.03 | 22.13 | 22.00 | 22.13 | 21.97 | 21.77 | KP1 |
| Mean | 31.57 | 30.03 | 30.13 | 29.87 | 29.97 | 29.63 | 28.87 | 30.13 | 30.47 | LK03 |
| Corpuscular | | | | | | | | | | |
| Hemoglobin | | | | | | | | | | |
| | 29.90 | 29.67 | 29.97 | 29.90 | 29.40 | 29.20 | 28.53 | 29.73 | 29.83 | KP1 |
| Platelet Count | 214.00 | 345.67 | 217.67 | 290.33 | 379.33 | 383.00 | 335.67 | 365.33 | 384.00 | LK03 |
| | 297.67 | 182.00 | 259.00 | 328.00 | 365.00 | 336.33 | 315.67 | 351.33 | 263.67 | KP1 |
| Absolute | 47.00 | 52.33 | 50.67 | 62.33 | 73.33 | 51.67 | 40.00 | 52.00 | 37.33 | LK03 |
| Reticulocyte | | | | | | | | | | |
| | 45.00 | 50.00 | 42.00 | 50.67 | 67.00 | 44.33 | 39.67 | 32.33 | 44.33 | KP1 |
| Percent | 0.73 | 0.97 | 1.10 | 1.23 | 1.37 | 0.93 | 0.67 | 0.90 | 0.63 | LK03 |
| Reticulocytes | | | | | | | | | | |
| | 0.73 | 0.87 | 0.73 | 0.90 | 1.20 | 0.73 | 0.67 | 0.53 | 0.70 | KP1 |
| White Blood | 14.17 | 14.40 | 9.63 | 12.03 | 13.57 | 13.33 | 14.63 | 13.43 | 12.93 | LK03 |
| Cell Count | | | | | | | | | | |
| | 7.77 | 7.40 | 6.50 | 6.13 | 7.70 | 6.30 | 6.40 | 7.30 | 6.27 | KP1 |
| Neutrophils | 4962.67 | 6116.67 | 4441.33 | 3802.67 | 5261.00 | 5392.33 | 5861.33 | 4733.00 | 4345.33 | LK03 |
| | 3395.33 | 3912.67 | 3300.33 | 2292.33 | 3905.67 | 1452.27 | 2202.33 | 3717.33 | 1962.67 | KP1 |
| Lymphocytes | 8545.33 | 7587.33 | 4698.33 | 7475.33 | 7544.67 | 7241.00 | 8151.67 | 8064.33 | 7813.33 | LK03 |
| | 3835.33 | 3038.33 | 2674.33 | 3297.67 | 3304.33 | 2093.27 | 3820.00 | 3076.00 | 3846.67 | KP1 |
| Monocytes | 474.00 | 608.00 | 433.67 | 688.33 | 639.33 | 509.33 | 483.33 | 487.33 | 603.33 | LK03 |
| | 462.00 | 394.00 | 479.67 | 492.00 | 426.67 | 285.07 | 351.67 | 444.00 | 340.33 | KP1 |
| Eosinophils | 118.33 | 74.00 | 15.00 | 50.00 | 105.33 | 97.67 | 31.33 | 126.00 | 155.33 | LK03 |
| | 66.33 | 52.33 | 41.33 | 41.67 | 58.33 | 32.40 | 23.33 | 53.67 | 107.67 | KP1 |
| Basophils | 66.00 | 14.33 | 45.33 | 16.67 | 16.33 | 93.33 | 106.00 | 23.00 | 16.33 | LK03 |
| | 7.67 | 2.67 | 4.33 | 10.00 | 5.00 | 3.33 | 3.00 | 9.33 | 9.67 | KP1 |

6. Coagulation Parameters

For assessing the coagulation parameters, set forth in Table 76, approximately 1.8 mL whole blood was collected from each animal at each of 7 days prior to AAV infusion, and at days 3, 7, 14, 28, 42, 56 and 84 post-infusion as detailed above and placed into tubes containing 3.2% sodium citrate as an anticoagulant. Samples were maintained at room temperature and centrifuged at 2500×g for 10 minutes at room temperature within one hour of collection. Plasma was separated and frozen at approximately −70° C. for batched analysis within six months of collection. Sample collection and processing was performed per the laboratory SOP for Collection and Processing of Samples for Clinical Pathology

Samples were analyzed for prothrombin time (PT) and activated partial thromboplastin time (aPTT) by automated mechanical clot formation analysis on an Amax Destiny Plus Coagulation Analyzer (Trinity Biotech, Jamestown, N.Y.) per manufacturers instructions on the Amax Destiny Plus Coagulation Analyzer (Trinity Biotech PLC, Bray, Co Wicklow, Ireland). The coagulation parameters that were assessed are shown in Table 76, below:

TABLE 76
Coagulation Parameters

| Parameter | Abbreviation^{A} | Units |
|---|---|---|
| Prothrombin time | PT | sec |
| Activated partial | aPTT | sec |
| thromboplastin time | | |

The results show that Prothrombin and Activated partial thromboplastin time are similar between LK03 and KP1 for all timepoints, and did not significantly change before and after AAV administration, demonstrating that PT and aPTT were unimpacted by AAV administration. Individual animal results also were assessed. The results do not indicate significant variation in PT or aPTT between and among individual animals.

The data are set forth as the mean of 2-3 samples in Table 77, and the individual animal data are set forth in Table 78, below:

TABLE 77
Coagulation Results

| | Day | | | | | | | | |
|---|---|---|---|---|---|---|---|---|---|
| Parameter | −7 | −3 | 7 | 14 | 28 | 42 | 56 | 84 | AAV |
| Prothrombin | 13.75 | nd | 13.55 | 12.3 | 13.37 | 14.1 | 12.93 | 12.73 | LK03 |
| time | 14.2 | 13.4 | 13.57 | 12.6 | 13.07 | 14.23 | 13.77 | 13.30 | KP1 |
| Activated partial | 25.15 | nd* | 27.25 | 25.43 | 25.73 | 24.57 | 25.67 | 25.93 | LK03 |
| thromboplastin | 24.85 | 20.3 | 23.63 | 23 | 24.87 | 28.33 | 28.63 | 24.60 | KP1 |
| time | | | | | | | | | |

*nd = not determined (Tables 77 and 78)

TABLE 78
Select Results for Individual Animals

| | Day | | | | | | | | | |
|---|---|---|---|---|---|---|---|---|---|---|
| Analyte | NHP No. | −7 | −3 | 7 | 14 | 28 | 42 | 56 | 84 | AAV |
| Prothrombin | 5 | 13.2 | nd* | nd | 12.3 | 13.4 | 13.3 | 12.2 | 12.9 | LK03 |
| time | 7 | nd | nd | 13.1 | 12.3 | 13.2 | 13.9 | 13.7 | 12.6 | |
| | 1 | 14.3 | nd | 14.0 | 12.4 | 13.5 | 12.0 | 12.9 | 12.7 | |
| | 3 | 15.8 | nd | 14.5 | 13.7 | 15.6 | 15.1 | 16.6 | 15.3 | KP1 |
| | 6 | 12.6 | nd | 12.5 | 11.4 | 12.4 | 11.6 | 12.3 | 12.4 | |
| | 9 | nd | 13.4 | 13.7 | 12.7 | 14.3 | 16.0 | 12.4 | 12.2 | |
| Activated | 5 | 20.5 | nd | nd | 25.6 | 25.0 | 24.7 | 21.8 | 21.6 | LK03 |
| partial | 7 | nd | nd | 23.7 | 21.1 | 23.0 | 23.4 | 23.2 | 22.0 | |
| thromboplastin | 1 | 29.8 | nd | 30.8 | 29.6 | 29.2 | 25.6 | 31.7 | 34.2 | |
| time | 3 | 24.0 | nd | 22.4 | 22.6 | 23.8 | 21.4 | 23.1 | 23.6 | KP1 |
| | 6 | 25.7 | nd | 28.2 | 25.3 | 29.9 | 39.1 | 26.6 | 28.2 | |
| | 9 | nd | 20.3 | 20.3 | 21.0 | 20.9 | 24.5 | 21.2 | 22.0 | |

7. FIX Expression

For analysis of FIX expression after rAAV-FIX administration, blood was collected from the femoral, saphenous, or cephalic veins of sedated cynomolgus monkeys at 7 days prior to the AAV injections, on the day of AAV dosing (day 0), and 3, 7, 14, 28, 42, 56 and 84 days post-intravenous dosing. Plasma was prepared from blood collection for bioanalytical assays. Sodium citrate was added to blood collected to 0.4% of the final volume. Plasma was prepared by centrifugation at 10,000 RPM at 4° C. for ten minutes and treated blood samples were stored at −80° C. Blood and plasma samples were kept on ice throughout collection and processing.

FIX concentrations in plasma were determined using a FIX enzyme-linked immunosorbent antigen assay (ELISA), as detailed in Example 14, above. The assay employed coating of anti-human Factor IX antibody (AHIX-5041, Heamatologic Technologies, Essex Vt.) at 2 ug/ml on a 96 well assay plate to capture the FIX. FIX antigen was detected with human-specific detection antibodies to avoid cross-reactivity with endogenous monkey FIX. Detection of the captured FIX was performed with a goat anti-human FIX (GAFIX-HRP, Affinity Biologicals, Ontario Canada) polyclonal antibody at 2 μg/mL conjugated to HRP, which emits a colorimetric signal directly proportional to the quantity of FIX. The colorimetric signal was measured on a Spectra MAX UV/VIS with SOFTmax PRO (Molecular Devices, San Jose, Calif.) and the unknown FIX concentrations in plasma were interpolated from a standard curve ranging from 0.4 ng/mL to 800 ng/mL of FIX.

FIX expression was calculated as an average of each animal at each time point analyzed in triplicate. Data are provided in Tables 79 and 80, below. Table 79 depicts rAAV-FIX expression relative to normal FIX expression in human. The results in Table 80 are displayed as FIX protein expression in ng protein/mL serum, without background subtracted out.

The results show that infusion of rAAV KP1-FIX and LK03-FIX achieved high initial FIX levels at 3 days and 1 week post infusion, which decreased to a steady plateau over the course of six weeks. The results show that no significant differences in plasma FIX levels were observed between animals administered rAAV-LK03-FIX and rAAV-KP1-FIX, demonstrating in vivo effectiveness of the AAV vectors for expression of FIX as provided herein.

TABLE 79
Modified FIX Protein Expression (% of normal human)
after infusion with LK03-FIX or KP1-FIX

NHP Animal Identification Number

| Day | 1 | 5 | 7 | 3 | 6 | 9 |
|---|---|---|---|---|---|---|
| −7 | 0 | 0 | 0 | 0 | 0 | 0 |
| 0 | 0 | 0 | 0 | 0 | 0 | 0 |
| 3 | 5.6 | 1.36 | 3.88 | 3.98 | 0.266 | 5.98 |
| 7 | 3.12 | 1.562 | 3.82 | 2.36 | 0.39 | 3.04 |
| 14 | 2.24 | 1.642 | 2.58 | 1.956 | 0.656 | 2.66 |
| 28 | 2.62 | 1.244 | 2.18 | 1.396 | 0.376 | 0.384 |
| 42 | 2.54 | 0.610 | 1.562 | 1.188 | 0.342 | BLQ |

*NHPs 1, 5, 7 were administered LK03-FIX; NHPs 3, 6, 9 were administered KP1-FIX BLQ = below quantitation limit of the assay

TABLE 80
Modified FIX Protein Expression (ng/mL)
after infusion with LK03-FIX or KP1-FIX

NHP Animal Identification Number

| Day | 1 | 5 | 7 | 3 | 6 | 9 |
|---|---|---|---|---|---|---|
| −7 | 0 | 0 | 0 | 0 | 0 | 0 |
| 0 | 0 | 0 | 0 | 0 | 0 | 0 |
| 3 | 280 | 68 | 194 | 199 | 13.3 | 299 |
| 7 | 156 | 78.1 | 191 | 118 | 19.5 | 152 |
| 14 | 112 | 82.1 | 129 | 97.8 | 32.8 | 133 |
| 28 | 131 | 62.2 | 109 | 69.8 | 18.8 | 19.2 |
| 42 | 127 | 30.5 | 78.1 | 59.4 | 17.1 | BLQ |
| 56 | 266 | 0 | 100 | 42.9 | 16.8 | BLQ |
| 84 | 0 | 10.2 | 87.5 | 0 | 16.5 | BLQ |

*NHPs 1, 5, 7 were administered LK03-FIX; NHPs 3, 6, 9 were administered KP1-FIX
BLQ = below quantitation limit of the assay

8. FIX Activity

For analysis of FIX activity after rAAV-FIX administration, blood was collected from the femoral, saphenous, or cephalic veins of sedated cynomolgus monkeys at 7 days prior to the AAV injections, and 3, 7, 14, 28, 42, 56 and 84 days post-intravenous dosing, and prepared as plasma for assessment of functional coagulation activity of modified FIX.

FIX activity was assessed using the activated partial thromboplastin time (aPTT)-based factor IX single-stage clotting assay primarily as detailed above, in Example 14. Briefly, the aPTT-based factor IX single-stage clotting assay was performed on an ACL-TOP instrument (Instrumentation Laboratories (Bedford, Mass.)) using standard reagents purchased from the manufacturer. Briefly, the aPTT reagents (HemosIL or SynthasIL) was equilibrated at 37° C. and preincubated with NHP plasma dilutions in accord with manufacturer's instructions. Clotting was induced through the automated addition of 20 mM calcium chloride to trigger the coagulation process. The clot was detected by measuring the change in optical density and the amount of time required for the plasma specimen to clot was recorded. Calibration was performed with the HemosIL Cal Plasma (Instrumentation Laboratories, Bedford, Mass.) as a reference traceable to the WHO standard (09/172). Each sample was run in true duplicate and using the required number of dilutions to obtain valid and reportable results. Sample dilutions generally ranged from 1:10 to 1:300 depending on the anticipated FIX expression levels.

Data are provided in Tables 81 and 82, below. FIX activity was calculated as an average of each animal at each time point analyzed in technical triplicate. Table 81 depicts relative rAAV-FIX percent activity changes compared to baseline hFIX activity in each individual animal with background (pre-treatment) FIX levels subtracted out. The results in Table 82 are displayed as FIX activity in IU/mL serum, without background (pre-treatment) FIX levels subtracted out.

The results show that FIX activity, as determined by the activated partial thromboplastin time (aPTT)-assay, was increased after administration of rAAV-LK03-FIX and rAAV-KP1-FIX. Infusion of rAAV KP1-FIX and LK03-FIX achieved high initial FIX activity directly after administration, and at 3 days up to two weeks post infusion. Thereafter, activity decreased to a steady plateau over the course of six weeks. The results show no significant differences in plasma FIX activity between animals administered rAAV-LK03-FIX and rAAV-KP1-FIX. In this model, modified FIX administered intravenously when packaged with the capsid of SEQ ID NO: 418 (KP1) and the LK03 capsid showed increased activity compared to baseline levels.

TABLE 81
Modified FIX Activity after infusion
(%) with LK03-FIX and KP1-FIX

NHP Animal Identification Number

| Day | 1 | 5 | 7 | 3 | 6 | 9 |
|---|---|---|---|---|---|---|
| −7 | 0 | 1.5 | 5.1 | 0 | 0 | 3.2 |
| 3 | 118.3 | 36.9 | 84 | 90.1 | 13.7 | 149 |
| 7 | 51.8 | 36.4 | 87.5 | 46.8 | 7.6 | 65.9 |
| 14 | 37.6 | 29.6 | 43.6 | 24.7 | 0.2 | 48.5 |
| 28 | 44.9 | 17.3 | 37.5 | 16.1 | 0.2 | 3.3 |
| 42 | 43.7 | 0.2 | 24.1 | 11.2 | 0 | 0 |

*NHPs 1, 5, 7 were administered LK03-FIX; NHPs 3, 6, 9 were administered KP1-FIX

TABLE 82
Modified FIX Activity (IU/mL) after
infusion with LK03-FIX and KP1-FIX

| | NHP Animal Identification Number | | | | | |
|---|---|---|---|---|---|---|
| Day | 1 | 5 | 7 | 3 | 6 | 9 |
| −7 | 0.2875 | 0.3675 | 0.404 | 0.3305 | 0.3425 | 0.385 |
| 3 | 1.536 | 0.722 | 1.193 | 1.254 | 0.4895 | 1.843 |
| 7 | 0.8705 | 0.717 | 1.2275 | 0.821 | 0.429 | 1.0115 |
| 14 | 0.7285 | 0.6485 | 0.789 | 0.5995 | 0.3545 | 0.8375 |
| 28 | 0.802 | 0.5255 | 0.728 | 0.514 | 0.3545 | 0.3855 |
| 42 | 0.7895 | 0.3545 | 0.5935 | 0.465 | 0.312 | 0.22 |
| 56 | 1.7305 | 0.3060 | 0.7515 | 0.4220 | 0.3790 | 0.2480 |
| 84 | 0.1770 | 0.2300 | 0.7215 | 0.2320 | 0.3485 | 0.2565 |

*NHPs 1, 5, 7 were administered LK03-FIX; NHPs 3, 6, 9 were administered KP1-FIX

9. FIX Specific Activity

The specific activity of modified FIX in animals intravenously infused with AAV-FIX (LK03 and KP1) was determined by the assessment of antigen levels and protein coagulation activity. The FIX-specific activity was calculated by dividing the clotting activity (IU/mL) by the antigen levels (ng/mL) and expressing the results in units per nanogram (IU/ng).

The results show that the specific activity of modified FIX was initially highest for modified FIX for all animals of both rAAVs administered. Thus, KP1, which was selected for and demonstrated high expression in human liver cells, demonstrates at least similar specific activity as the highly active rAAV-LK03, which was previously shown to efficiently transduce NHP cells (Wang et al., Mol. Ther. (2015) 23(12):1877-1887). Wang et al. notes that although rAAV-LK03 efficiently transduces NHP cells, the transduction efficiency was less dramatic than the transduction efficiency of human cells, for which they were selected as transducing better than previously characterized chimeric capsids. Similar results, thus, should be observed for rAAV-KP1, which was also selected for in human hepatocytes. The FIX specific activity results are set forth in Table 83, below:

TABLE 83
Specific Activity (IU/ng) of modified FIX
after infusion with LK03-FIX or KP1-FIX

| | NHP Animal Identification Number | | | | | |
|---|---|---|---|---|---|---|
| Day | 1 | 5 | 7 | 3 | 6 | 9 |
| 3 | 0.00446 | 0.00521 | 0.00407 | 0.00464 | 0.01105 | 0.004876 |
| 7 | 0.00374 | 0.00448 | 0.00431 | 0.00416 | 0.00444 | 0.004122 |
| 14 | 0.00394 | 0.00342 | 0.00298 | 0.00275 | 0.00037 | 0.003402 |
| 28 | 0.00393 | 0.00254 | 0.00297 | 0.00263 | 0.00064 | 0.000026 |
| 42 | 0.00395 | BLQ | 0.00243 | 0.00226 | BLQ | BLQ |
| 56 | 0.00543 | BLQ | 0.00348 | 0.00213 | 0.00217 | BLQ |
| 84 | BLQ | BLQ | 0.00363 | BLQ | 0.00036 | BLQ |

*NHPs 1, 5, 7 were administered LK03-FIX; NHPs 3, 6, 9 were administered KP1-FIX
BLQ = below quantitation limit of the assay

10. Conclusions

rAAV-KP1 was generated and selected for high expression in human liver cells. These results are replicated in NHPs, where, upon intravenous administration of rAAV-KP1-FIX, NHPs exhibited high initial FIX expression and activity. Expression of the modified FIX (R318Y/R338E/T343 replacements) described herein, which has high potency, was similar between the KP1 capsid described herein and the rAAV-LK03, which was shown to effectively transduce human hepatocytes, and to a lesser extent NHPs. It can be inferred from this evidence that the KP1 capsid and rAVV expression vector containing the KP1 capsid and encoding a modified FIX, with an exon as described herein, will be expressed in humans and provide high potency FIX in vivo.

Animal Welfare

Only visually healthy animals were enrolled in the study. This study complied with all applicable sections of The Animal Welfare Act Regulations (9 CFR Parts 1, 2, 3) and the Guide for the Care and Use of Laboratory Animals, Eight Edition National Research Council, National Academy Press Washington, D.C. Copyright 2011. All assessments took place in an animal research facility this is fully accredited by the Association for Assessment and Accreditation of Laboratory Animal Care (AAALAC).

### Example 19

### FIX mRNA Detection in Cynomolgus Monkey Liver after Injection of the rAAV-KP1 Vector with the Capsid KP1, and the Vector Designed rAAV-LK03, Each Encoding Modified FIX (R318Y/R338E/T343R)

RNA detection experiments were performed to determine the number of RNA molecules in liver sections from cynomolgus monkeys injected with 1.0e12 vg/kg of AAV-modified FIX (SEQ ID NO:486, or SEQ ID NO:394 with the replacement T148A). Liver samples were assayed for detection of human FIX transgene (codon optimized human FIX).

Animals were euthanized and liver samples were collected for RNA analyses for detection of the human FIX codon optimized transgene. Briefly, Formalin-Fixed Paraffin-Embedded (FFPE) samples from the left, right, caudate, and quadrate liver lobes were sectioned at 5 μm thickness and mounted onto SuperFrost Plus slides (Fisher Scientific; 12-550-15) in a water bath. Six samples were collected from each animal, and 12 replicate slides with 4 sections per slide were prepared for each animal.

RNAscope® in situ hybridization was performed on liver sections and analyzed using the RNAscope® 2.5 LSx Red Reagent Kit-RED (Advanced Cell Diagnostics, Inc.; Cat. No. 322750), generally in accord with manufacturer's instructions. This method hybridizes gene-specific probe pairs to target mRNA (here, codon optimized, modified hFIX (R318Y/R338E/T343R)). The signal then was amplified in a series of steps, hybridized to a final alkaline phosphatase labeled probe, and detected using Fast Red. Each RNA transcript of interest was discerned as an individual chromogen dot under brightfield microscopy. Scanned images were visually scored for transcript levels based on number of dots per cell.

Preliminary experiments were conducted to optimize pretreatment conditions and to identify conditions to maximize signal-to-noise ratio. Samples were assessed as per the kit instructions (Advanced Cell Diagnostics, Inc.; Cat. No. 322750) with the following differences: epitope retrieval 2 (LSER2) was extended to 20 minutes at 95° C., and the Protease III step was extended to 20 minutes at 40° C. The positive control probe target was Macaca fascicularis ubiquitin C (UBC) transcript variant X1 (Mfa-UBC) (Advanced Cell Diagnostics, Inc.; Cat. No. 461338). The negative control probe target was B. subtilis gene dihydrodipicolinate reductase (dapB) (Advanced Cell Diagnostics, Inc.; Cat. No. 312038), which is not present in cynomolgus monkey liver samples.

Samples were scored visually by a qualified scientist who assigned a single score to a sample based on the predominant staining pattern throughout the entire sample. Heterogeneity or non-uniformity of RNA staining was noted, if applicable. Samples were assessed for the number of foci per cell, ignoring the intensity of the staining, which does not correspond to the RNA quantification. Foci correlate to the number of individual RNA molecules, whereas dot intensity reflects the number of probe pairs bound to each molecule. Categories were established as set forth in Table 84, below:

TABLE 84
Scoring Criteria

| Score | Criteria |
|---|---|
| 0 | No staining or <1 dot/10 cells |
| 1 | 1-3 dots/cell |
| 2 | 4-9 dots/cell, with no or very few dot clusters |
| 3 | 10-15 dots/cell and/or <10% dots are in clusters |
| 4 | >15 dots/cell and/or >10% dots are in clusters |

Samples also were scored for the percentage of cells with target transcripts; categories were established as follows: 0%, 1-25%, 26-50%, 51-75%, 76-99%, or 100% positive cells. Samples also were assessed visually for the percent of positive cells that exhibited more than one foci per cell; categories were established as follows: <1%, 1-25%, 26-50%, or >50% cells with more than one foci per cell.

Higher staining of the Mfa-UBC positive control was noted around the edges of the tissue relative to the center. To mitigate this heterogeneity in staining, which is likely due to improper tissue fixation, pretreatment conditions were extended, which resulted in all samples passing internal quality control, where moderate to high UBC positive control staining was obtained throughout the sample, with little to no dapB staining (negative control).

As an additional control, the RNAscope® 2.5 LSx Red Assay was performed in pelleted liver cells. The results show that FIX was detected at high levels in a subset of liver cells in samples from NHP animal numbers 6 (KP1) and 7 (LK03). Cells from animals 1, 3, 5, and 9 had negligible to low detection of the transgene. The RNA expression results from pelleted liver cells correspond to the results from the fixed liver samples, detailed below.

The results for immunohistological staining are set forth in Table 85, below. The positive and negative controls gave expected results; the positive control staining was a score between 3 and 4 for almost all samples, demonstrating the high quality of the preparation and protocol, and the negative control (dapB score) was 0 for all samples, indicating a lack of non-specific staining. The results show that NHP animal numbers 6 and 7 were scored as 4 in each of the samples tested, exhibiting >15 dots per cell and/or >10% of dots in clusters, which indicates high RNA expression in a subset of cells. Animal number 6 was transduced with the KP1-FIX AAV vector, and animal number 7 was transduced with the LK03-FIX AAV vector. Thus, one animal for each vector was effectively transduced and exhibited high FIX RNA expression in liver. The other NHP animals (numbers 1, 5, 3 and 9) showed little to no FIX mRNA, and were all scored as a “1.” These results correlate with the FIX antigen levels at the end of study (see e.g., Table 80 day 84). Duplicate slides were assessed for each condition, and the results were the same for both slides of the corresponding condition. The results set forth in Table 85, below, represent the results for both slides.

TABLE 85
mRNA molecules in liver cells in animals
transduced with FIX AAV vectors

| | NPH | | | FIX | |
|---|---|---|---|---|---|
| Vector | animal | Core | Positive Control | Score | % FIX positive cells |
| LK03 | 1 | 1 | 4 | 1 | 1-25% |
| | | 2 | 4 | 1 | 1-25% |
| | | 3 | 4 | 1 | 1-25% |
| | | 4 | 4 | 1 | <1% |
| | 5 | 1 | 3-4* | 1 | 1-25% |
| | | 2 | 3-4 | 1 | <1% |
| | | 3 | 3-4 | 1 | <1% |
| | | 4 | 2 | 1 | <1% |
| | 7 | 1 | 4 | 4 | 1-25% |
| | | 2 | 3-4* | 4* | 1-25% |
| | | 3 | 3-4* | 4* | 1-25% |
| | | 4 | 3-4* | 4 | 1-25% |
| KP1 | 3 | 1 | 3-4 | 1 | 1-25% |
| | | 2 | 2* | 1* | 1-25% |
| | | 3 | 4 | 1* | <1% |
| | | 4 | 2 | 1* | 1-25% |
| | 6 | 1 | 4* | 4 | <1% |
| | | 2 | 4* | 4 | <1% |
| | | 3 | 4* | 4 | <1% |
| | | 4 | 4* | 4 | <1% |
| | 9 | 1 | 4 | 1 | 1-25% |
| | | 2 | 3-4* | 1 | 1-25% |
| | | 3 | 4* | 1* | 1-25% |
| | | 4 | 4 | 1 | 1-25% |

*Staining along the edges was higher than the center of the tissue

The results show LK03-AAV NHP numbers 1 and 5 and KP1 NHP number 3 showed high ECL values and were presumed positive. The positive results were confirmed for these animals. NHP animal numbers 6 (KP1) and 7 (LK03) were negative at all timepoints. Data are set forth in tables 86 and 87, below:

TABLE 86
LK03

| | NHP Animal Identification Number | | | | | | | | | | | |
|---|---|---|---|---|---|---|---|---|---|---|---|---|
| | 1 | | | | 5 | | | | 7 | | | |
| | | | | Conf. | | | | Conf. | | | | Conf. |
| Day | ECL | Result | Conf. | Result | ECL | Result | Conf. | Result | ECL | Result | Conf. | Result |
| −7 | 76 | Negative | NA | NA | 72 | Negative | NA | NA | 76 | Negative | NA | NA |
| 0 | 78 | Negative | NA | NA | 68 | Negative | NA | NA | 68 | Negative | NA | NA |
| 3 | 75 | Negative | NA | NA | 73 | Negative | NA | NA | 74 | Negative | NA | NA |
| 7 | 78 | Negative | NA | NA | 74 | Negative | NA | NA | 72 | Negative | NA | NA |
| 14 | 92 | Pres. | 6.99 | Negative | 77 | Negative | NA | NA | 73 | Negative | NA | NA |
| | | Positive | | | | | | | | | | |
| 28 | 89 | Pres. | −0.64 | Negative | 100 | Pres. | 32.41 | Positive | 73 | Negative | NA | NA |
| | | Positive | | | | Positive | | | | | | |
| 42 | 97 | Pres. | 16.37 | Positive | 1722 | Pres. | 96.87 | Positive | 82 | Negative | NA | NA |
| | | Positive | | | | Positive | | | | | | |
| 56 | 146 | Pres. | 61.21 | Positive | 8550 | Pres. | 99.29 | Positive | 78 | Negative | NA | NA |
| | | Positive | | | | Positive | | | | | | |
| 84 | 7871 | Pres. | 99.20 | Positive | 18061 | Pres. | 99.57 | Positive | 100 | Pres. | 3.62 | Neg |
| | | Positive | | | | Positive | | | | Positive | | |
| 98 | 16199 | Pres. | 99.60 | Positive | 15195 | Pres. | 99.56 | Positive | 92 | Pres. | 3.11 | Neg |
| | | Positive | | | | Positive | | | | Positive | | |

TABLE 87
KP1

| | NHP Animal Identification Number | | | | | | | | | | | |
|---|---|---|---|---|---|---|---|---|---|---|---|---|
| | 3 | | | | 6 | | | | 9 | | | |
| Day | ECL | Result | Conf. | ECL | Result | Conf. | ECL | Result | Conf. | ECL | Result | Conf. |
| −7 | 62 | Negative | NA | NA | 68 | Negative | NA | NA | 71 | Negative | NA | NA |
| 0 | 61 | Negative | NA | NA | 68 | Negative | NA | NA | 70 | Negative | NA | NA |
| 3 | 61 | Negative | NA | NA | 71 | Negative | NA | NA | 70 | Negative | NA | NA |
| 7 | 71 | Negative | NA | NA | 68 | Negative | NA | NA | 69 | Negative | NA | NA |
| 14 | 74 | Negative | NA | NA | 76 | Negative | NA | NA | 81 | Negative | NA | NA |
| 28 | 74 | Negative | NA | NA | 64 | Negative | NA | NA | 2254 | Negative | NA | Positive |
| 42 | 74 | Negative | NA | NA | 67 | Negative | NA | NA | 15306 | Pres. | 96.82 | Positive |
| | | | | | | | | | | Positive | | |
| 56 | 87 | Pres. | 18.75 | Positive | 62 | Negative | NA | NA | 21593 | Pres. | 99.42 | Positive |
| | | Positive | | | | | | | | Positive | | |
| 84 | 1796 | Pres. | 96.78 | Positive | 74 | Negative | NA | NA | 9876 | Pres. | 99.59 | Positive |
| | | Positive | | | | | | | | Positive | | |
| 98 | 8546 | Pres. | 99.27 | Positive | 78 | Negative | NA | NA | 71 | Pres. | 99.14 | NA |
| | | Positive | | | | | | | | Positive | | |

Pres. Positive = Presumptive Positive

### Example 20

### Vector Copy Numbers and FIX Transcripts in NHP Liver after Injection of a Recombinant Adeno-Associated Viral Vectors (rAAV), Provided Herein, Designated rAAV-LK03 and rAAV-KP1 Each Encoding Modified FIX (R318Y/R338E/T343R)

In vivo transduction efficiency of rAAV packaged with the capsid designated KP1 (SEQ ID NO:418) or LK03 ((Lisowski et al., (2014) Nature 506(7488):382-86), and expressing modified FIX (R318Y/R338E/T343R), was examined in non-human primate (NHP) animals 1, 3, 5, 6, 7 and 9, described in the Example above. huFIX transcript levels and rAAV vector geneome copy numbers were quantified in the livers of NHPs injected with the rAAV FIX vector packaged with the capsid KP1 or LK03 after sacrifice at Day 98 post rAAV injection.

The animals were sacrificed, livers were harvested, and the livers were prepared for analysis of vector copy number by qPCR, at two test sites, using different protocols. Both sites used the plasmid encoding modified FIX (R318Y/R338E/T343R) codon optimized for mouse to generate a standard curve of known FIX concentrations. The primers whose sequences are set forth in SEQ ID NOS: 567 and 568 were used as forward and reverse amplification primers, respectively.

At test site 1, the following protocol was followed. Total genomic DNA was isolated from 25 mg frozen liver samples from the left, right, caudate, and quadrate liver lobes using the MagMAX™-96 DNA Multi-Sample kit (Invitrogen; Cat. No. 4413021), per the manufacturer's instructions. An 8-point standard curve was prepared from circularized double stranded plasmid DNA with a known final copy number concentration from 5×10⁷ vg/μL-5 vg/μL. qPCR reactions were prepared with 300 nM each of FIX forward and reverse primers, 100 nM FIX probe, and 1× final concentration to TaqMan Fast Advanced Master Mix (Applied Biosystems; Cat. No. 4444557), and reactions were run per the cycle durations indicated in Table 88. Starting vector copy number was back calculated against the regression equation generated by the calibration curve and expressed in FIX copies/μg DNA.

TABLE 88
PCR Conditions

| | UNG | Initial | qPCR | |
|---|---|---|---|---|
| | Incubation | Denaturation | (40 cycles) | |
| Parameter | Hold | Hold | Denature | Anneal/Extend |
| Temperature | 50 | 95 | 95 | 60 |
| (° C.) | | | | |
| Time (mm:ss) | 2:00 | 2:00 | 0:03 | 0:30 |

At test site 2, the following protocol was followed. Total genomic DNA was isolated from 25 mg frozen liver samples from the left, right, caudate, and quadrate liver lobes. An 8-point standard curve was prepared from linearized double stranded FIX plasmid DNA with a known final copy number concentration from 10⁸ copies/μL-10 copies/μL. Similarly, 8-point standard curves also were generated from solutions of known albumin or hemoglobin gene copy number concentrations of 10⁸ copies/μL-10 copies/μL. Samples were run in triplicate for duplex qPCR reactions with either FIX/albumin standard curves and probes, or FIX/hemoglobin standard curves and probes. Resultant quantitation cycle (Cq) values for each product were back calculated against the regression equation generated by the calibration curve for its corresponding standard. Starting FIX vector copy numbers were then expressed as a ratio of FIX copies/albumin copies, FIX copies/hemoglobin copies, or FIX copies/μg DNA.

The results for vector copies/g of total genomic DNA (gDNA) for sites 1 and 2 showed similar trends, with differences in the raw values of total copy number per total amount of gDNA. The data from site 1 are set forth in Tables 89-96, below. The results show that vector copy numbers were similar between animals, with the exception of KP1 transduced NHP animal number 6, which showed decreased vector copy numbers, and KP1 transduced NHP animal number 9 and LK03 transduced NHP animal number 7, which both showed increased vector copy numbers compared to the other animals. The data for vector copy number/μg gDNA, for site 1, is set forth in Table 89, below. Similar trends were shown when the vector copy number was compared to albumin or hemoglobin gene copy numbers. These results are set forth in Tables 90 and 91 as mean vector copies per cell normalized to albumin copies or actin copies.

FIX transcript levels also were assessed relative to actin and albumin transcripts. A two-step RT-PCR method was performed. First, total RNA was isolated from frozen liver samples of the left, right, caudate, and quadrate liver lobes. Trace amounts of genomic DNA was removed. The resulting purified RNA was then reverse transcribed to cDNA as described below and run per the cycle durations indicated.

The cDNA template was then used in duplex qPCR reactions with either factor 9/albumin standard curves and probes or factor 9/actin standard curves and probes. In this way, resultant quantitation cycle (Cq) values for each product could be back calculated against the regression equation generated by the calibration curve for its corresponding standard. Starting factor 9 transcript numbers were then expressed as a ratio of factor 9 transcripts/albumin transcripts or factor 9 transcripts/actin transcripts.

The results are set forth as a percentage of actin (Table 93) and albumin (Table 92). The results show huFIX transcript levels were highest in NHP 7 (AAV-LK03). NHP 6 also showed increased FIX transcript levels relative to actin and albumin, compared to the other animals. The results are set forth in Tables 89-93, below:

TABLE 89
Vector Copy Number/μg gDNA (×10⁵)

| | LK03 | | | KP1 | | |
|---|---|---|---|---|---|---|
| | 1 | 5 | 7 | 3 | 6 | 9 |
| Left | 1.43 | 1.77 | 10.50 | 4.77 | 0.285 | 9.44 |
| Right | 3.80 | 2.35 | 9.80 | 4.09 | 0.658 | 12.40 |
| Caudate | 2.40 | 3.45 | 7.74 | 6.07 | 0.5380 | 10.90 |
| Quadrate | 4.77 | 2.60 | 7.69 | 2.67 | 0.478 | 11.60 |
| Average* | 3.10 | 2.54 | 8.94 | 4.40 | 0.49 | 11.10 |

*Average of all lobes

TABLE 90
Vector copy number/albumin gene copies

| | |
|---|---|
| LK03 | KP1 |

| | 1 | 5 | 7 | 3 | 6 | 9 |
|---|---|---|---|---|---|---|
| Left | 1.25 | 1.44 | 8.07 | 3.5 | 0.26 | 6.61 |
| Right | 3.03 | 1.72 | 7.19 | 3.17 | 0.61 | 8.83 |
| Caudate | 1.69 | 2.65 | 6.37 | 4.27 | 0.34 | 8.29 |
| Quadrate | 2.87 | 2.13 | 6.87 | 1.97 | 0.35 | 7.90 |
| Total | 2.21 | 1.98 | 7.13 | 3.23 | 0.40 | 7.91 |

TABLE 91
Vector copy number/hemoglobin gene copies

| | |
|---|---|
| LK03 | KP1 |

| | 1 | 5 | 7 | 3 | 6 | 9 |
|---|---|---|---|---|---|---|
| Left | 0.94 | 1.17 | 4.85 | 2.50 | 0.2 | 4.85 |
| Right | 1.84 | 1.41 | 6.03 | 2.55 | 0.39 | 5.87 |
| Caudate | 1.32 | 1.77 | 5.10 | 2.96 | 0.25 | 6.04 |
| Quadrate | 2.32 | 1.32 | 5.36 | 1.45 | 0.24 | 5.11 |
| Total | 1.60 | 1.42 | 5.33 | 2.37 | 0.27 | 5.46 |

TABLE 92
FIX transcripts Relative to Albumin Transcripts (×10⁻⁴)

| | LK03 | | | KP1 | | |
|---|---|---|---|---|---|---|
| | 1 | 5 | 7 | 3 | 6 | 9 |
| Left | 2 | 3.0 | 69.6 | 2.5 | 13.6 | 3.3 |
| Right | 6.5 | 2.4 | 69.3 | 2.1 | 18.1 | 5.4 |
| Caudate | 2.1 | 2.6 | 57.4 | 2.3 | 21.4 | 5.0 |
| Quadrate | 2.7 | 3.2 | 64.0 | 1.8 | 19.5 | 4.5 |
| Total | 3.3 | 2.8 | 65.1 | 2.2 | 18.2 | 4.5 |

TABLE 93
FIX transcripts Relative to Actin Transcripts (×10⁻²)

| | LK03 | | | KP1 | | |
|---|---|---|---|---|---|---|
| | 1 | 5 | 7 | 3 | 6 | 9 |
| Left | 10.2 | 22.7 | 232 | 12.9 | 18.2 | 3.3 |
| Right | 5.9 | 11.7 | 226 | 4.6 | 12.5 | 4.5 |
| Caudate | 4.7 | 22.9 | 198 | 10.4 | 15.9 | 4.2 |
| Quadrate | 6.7 | 12.0 | 165 | 6.4 | 20.1 | 4.7 |
| Total | 6.88 | 17.337 | 205 | 8.59 | 16.65 | 4.16 |

Next, FIX transcripts normalized to albumin or actin transcripts (% of albumin and % of actin, respectively) as a proportion of the number of vector copies, was calculated. This calculation estimates transcriptional efficiency, as it compares FIX transcripts to the total number of copies available within the tissue. The results follow similar trends to the results set forth above; huFIX transcript levels were highest in NHP 7 (AAV-LK03) and NHP 6 (AAV-KP1) also showed increased FIX transcript levels as a proportion of the number of vector copies. The results are set forth in Tables 94 and 95, below:

TABLE 94
FIX transcripts (% of Albumin)/Vector copies

| | LK03 | | | KP1 | | |
|---|---|---|---|---|---|---|
| | 1 | 5 | 7 | 3 | 6 | 9 |
| Left | 1.41E−09 | 1.71E−09 | 6.61E−09 | 5.27E−10 | 4.78E−08 | 3.46E−10 |
| Right | 1.71E−09 | 1.02E−09 | 7.07E−09 | 5.13E−10 | 2.75E−08 | 4.34E−10 |
| Caudate | 8.75E−10 | 7.44E−10 | 7.41E−09 | 3.79E−10 | 3.98E−08 | 4.55E−10 |
| Quadrate | 5.75E−10 | 1.22E−09 | 8.32E−09 | 6.66E−10 | 4.08E−08 | 3.91E−10 |
| Average | 1.14E−09 | 1.17E−09 | 7.35E−09 | 5.21E−10 | 3.9E−08 | 4.07E−10 |

TABLE 95
FIX transcripts (% of Actin)/Vector copies

| | LK03 | | | KP1 | | |
|---|---|---|---|---|---|---|
| | 1 | 5 | 7 | 3 | 6 | 9 |
| Left | 7.12E−07 | 1.28E−06 | 2.2E−06 | 2.71E−07 | 6.37E−06 | 3.5E−08 |
| Right | 1.56E−07 | 4.99E−07 | 2.29E−06 | 1.12E−07 | 1.89E−06 | 3.58E−08 |
| Caudate | 1.95E−07 | 6.62E−07 | 2.56E−06 | 1.71E−07 | 2.95E−06 | 3.82E−08 |
| Quadrate | 1.4E−07 | 4.64E−07 | 2.14E−06 | 2.41E−07 | 4.2E−06 | 4.08E−08 |
| Average | 3.01E−07 | 7.27E−07 | 2.3E−06 | 1.99E−07 | 3.85E−06 | 3.74E−08 |

Next, the proportion of FIX transcripts as a percentage of albumin compared to the number of vector copies per albumin copies, was calculated. The fact that these findings recapitulate those in tables 94 and 95 (relative trends compared among samples) evidences that the albumin gene copy number was consistent between total genomic DNA samples tested, as a set amount of gDNA should have the same number of albumin or globulin copies (2 per cell). The results tend to show that animals transduced with KP1-FIX had higher levels of FIX transcripts in liver as a proportion of vector and albumin copies. Animal #7, which was transduced with LK03-FIX also showed high levels.

TABLE 96
FIX transcripts (% albumin)/VC/albumin copies

| | LK03 | | | KP1 | | |
|---|---|---|---|---|---|---|
| | 1 | 5 | 7 | 3 | 6 | 9 |
| Left | 1.52E−07 | 1.85E−07 | 6.36E−07 | 5.01E−08 | 5.08E−06 | 3.65E−08 |
| Right | 1.57E−07 | 1.14E−07 | 7.7E−07 | 5.64E−08 | 2.6E−06 | 4.11E−08 |
| Caudate | 9.59E−08 | 6.96E−08 | 8.04E−07 | 3.58E−08 | 3.65E−06 | 4.9E−08 |
| Quadrate | 5.36E−08 | 1.15E−07 | 8.96E−07 | 6.2E−08 | 3.94E−06 | 3.59E−08 |
| Total | 1.15E−07 | 1.21E−07 | 7.76E−07 | 5.11E−08 | 3.82E−06 | 4.06E−08 |

The results show that rAAV vector copy numbers and FIX transcript levels varied among animals and high levels were observed with KP1 and with LK03. When FIX transcripts were calculated relative to albumin, taking into account the number of vector copies, KP1-transfected animals had a higher average of hFIX transcripts than LK03.

### Example 21

### FIX Protein Expression in Mouse Plasma after Injection of a Recombinant Adeno-Associated Viral Vector (rAAV), Provided Herein, Encoding Differentially Codon Optimized Modified FIX (R318Y/R338E/T343R)

The effects of various codon optimization schemes on FIX expression in vivo in a mouse model was examined. The most effective is selected to for expression in primates. The nucleotide sequence for expression of modified FIX (R318Y/R338E/T343R) was optimized differentially and three codon optimized FIX sequences, set forth as SEQ ID NOs: 569, 570 and 571, were assessed. AAV FIX-encoding vectors, using each of the three optimized FIX sequences, were packaged with the capsid KP1 (SEQ ID NO:418). C57BL/6 WT mice were injected with one of the three codon optimized FIX plasmids, and FIX expression was monitored for several weeks.

A. FIX Protein Expression in Plasma after Intravenous Injection of rAAV-Modified FIX in Wild-Type Mice

C57BL/6 WT mice (4 mice/group) were injected via the tail vein with 5 E+9 vector genomes per kg (vg/kg) (assuming an average mouse weight of 25 g) of rAAV FIX vector containing the modified FIX encoding nucleotide sequence of SEQ ID NO: 569, 570 and 571. The full FIX sequence including the flanked regions are set forth in SEQ ID NOs: 562-564, for SEQ ID NOs: 572-574, respectively. To summarize:

SEQ ID NO: 562 sets forth the sequence of intron-propeptide-mature human FIX R318Y/R338E/T343R; SEQ ID NO:563 sets for the sequence of intron-propeptide-mature human FIX R318Y/R338E/T343R; and SEQ ID NO:564 sets forth the sequence of intron-propeptide-mature human FIX R318Y/R338E/T343R. Each of SEQ ID NOS: 569-571 sets forth an optimized sequence for human FIX R318Y/R338E/T343R.

Whole blood was collected by phlebotomy of the retro orbital plexus at 1, 2, 3, 4, 5 and 6 weeks post-intravenous dosing and plasma was prepared for bioanalytical assays to assess modified FIX (R318Y/R338E/T343R) expression. Animal number 8, which was among those injected with the FIX encoded by SEQ ID NO: 573 group, died at 3 weeks post-injection. Thus, blood only was collected and analyzed for week 1 and 2 post-injection. Blood collected from the retro orbital plexus of each mouse was diluted in 3.8% sodium citrate and plasma was prepared by centrifugation at 13,000 RPM at 4° C. for three minutes and treated blood samples were placed on dry ice and stored at −80° C. Blood and plasma samples were kept on ice throughout collection and processing.

FIX concentrations in plasma were determined using a FIX enzyme-linked immunosorbent antigen assay (ELISA) specific for hFIX antigen (ASSERACHROM IX: Ag Enzyme Immunoassay for Factor FIX; Diagnostica Stago, France) essentially according to the manufacturer's instructions using known concentrations of recombinantly expressed modified FIX as controls. Wells of a 96-well plate were coated with 2 μg/mL of anti-human Factor IX antibody (AHIX-5041, Heamatologic Technologies, Essex Vt.) to capture the FIX. Detection of the captured FIX was performed with a goat anti-human FIX polyclonal antibody conjugated with HRP (GAFIX-HRP, Affinity Biologicals, Ontario Canada), at 2 μg/mL. Upon binding of FIX to the FIX antibody, a colorimetric signal was emitted; signal strength is directly proportional to the quantity of FIX. The colorimetric reaction was measured on a Spectra MAX UV/VIS with SOFTmax PRO (Molecular Devices, San Jose, Calif.), and the unknown FIX concentrations in plasma were interpolated from a standard curve ranging from 0.4 ng/mL to 800 ng/mL of recombinantly expressed variant.

The results show that codon optimized FIX set forth in SEQ ID NOs: 570 and 571 expressed higher in mice than the codon optimized FIX set forth in SEQ ID NO:569. Modified FIX expression (in ng/mL) increased over the six weeks post vector-injection in mice administered the codon optimized FIX set forth in SEQ ID NOs: 570 and 571; whereas modified FIX expression of mice administered the codon optimized FIX set forth in SEQ ID NO: 569 remained stable over the trial period. The modified FIX expression results for each of the 12 individual animals (4 animals per optimized FIX sequence) are set forth in Table 97 below. The average FIX expression for each group of animals administered a vector containing codon optimized modified FIX set forth in SEQ ID NOs: 569-571 are set forth in Table 98, below. Units in each table are μg/mL.

TABLE 97
Modified FIX (R318Y/R338E/T343R) Expression in Individual Animals

| Time | Optimized FIX #1 | | | | Optimized FIX #2 | | | | Optimized FIX #3 | | | |
|---|---|---|---|---|---|---|---|---|---|---|---|---|
| after | (SEQ ID NO: 569) | | | | (SEQ ID NO: 570) | | | | (SEQ ID NO: 571) | | | |
| AAV | Mouse | Mouse | Mouse | Mouse | Mouse | Mouse | Mouse | Mouse | Mouse | Mouse | Mouse | Mouse |
| injection | 1 | 2 | 3 | 4 | 5 | 6 | 7 | 8 | 9 | 10 | 11 | 12 |
| 1 weeks | 2.12 | 2.04 | 2.76 | 1.71 | 3.19 | 2.67 | 3.11 | 4.84 | 2.98 | 2.61 | 3.65 | 3.19 |
| 2 weeks | 2.96 | 2.48 | 3.96 | 2.4 | 5.01 | 3.95 | 5.04 | 4.08 | 5.44 | 5.59 | 6.2 | 4.63 |
| 3 weeks | 2.63 | 2.89 | 3.89 | 2.79 | 6 | 3.36 | 5.93 | n/a | 5.56 | 4.41 | 6.01 | 5.21 |
| 4 weeks | 2.19 | 1.91 | 3.5 | 2.54 | 4.38 | 3.71 | 4.31 | n/a | 4.28 | 3.78 | 5.65 | 4.39 |
| 5 weeks | 2.25 | 1.77 | 3.44 | 2.68 | 4.56 | 3.66 | 4.8 | n/a | 4.63 | 3.95 | 7.04 | 5.44 |
| 6 weeks | 2.31 | 1.88 | 3.19 | 2.51 | 9.24 | 4.2 | 7.08 | n/a | 5.38 | 3.58 | 7.55 | 5.82 |

TABLE 98
Average Modified FIX (R318Y/R338E/T343R) Expression

| | Optimized FIX 1 | Optimized FIX 2 | Optimized FIX 3 |
|---|---|---|---|
| | (SEQ ID NO: 569 | (SEQ ID NO: 570) | (SEQ ID NO: 571) |
| Week 1 | 2.16 | 3.45 | 3.11 |
| Week 2 | 2.95 | 4.52 | 5.47 |
| Week 3 | 3.05 | 5.10 | 5.30 |
| Week 4 | 2.54 | 4.13 | 4.53 |
| Week 5 | 2.54 | 4.34 | 5.27 |
| Week 6 | 2.47 | 6.84 | 5.58 |

### Example 22

### Summary of Exemplary Vectors, Constructs, and Components Thereof

| SEQ | FIX or | | |
|---|---|---|---|
| ID | Construct or | Domains as in SEQ | |
| NO | Vector | Listing | Description |
| 569 | mature FIX | | mature human FIX |
| | optimized 1 | | R318Y/R338E/T343R optimized 1 |
| 570 | mature FIX | | mature human FIX |
| | optimized 2 | | R318Y/R338E/T343R optimized 2 |
| 571 | mature FIX | | mature human FIX |
| | optimized 3 | | R318Y/R338E/T343R optimized 3 |
| 562 | intron-pro-FIX | <221> intron | intron-propeptide-mature human FIX |
| | (optimized 1) | <222> (1) . . . (163) | R318Y/R338E/T343R optimized 1 |
| | | <221> propeptide | |
| | | <222> (164) . . . (214) | |
| | | <221> exon | |
| | | <222> (215) . . . (1459) | |
| | | <221> cloning vector | |
| | | <222> (1468) . . . (1857) | |
| 563 | intron-pro-FIX | same as above | intron-propeptide-mature human FIX |
| | (optimized 2) | | R318Y/R338E/T343R optimized 2 |
| 564 | intron-pro-FIX | same as above | intron-propeptide-mature human FIX |
| | (optimized 3) | | R318Y/R338E/T343R optimized 3 |
| 565 | intron-pro-FIX | <221> intron | intron-propeptide-mature human FIX |
| | | <222> (1) . . . (163) | R318Y/R338E/T343R not optimized |
| | | <221> propeptide | |
| | | <222> (164) . . . (214) | |
| | | <221> exon | |
| | | <222> (215) . . . (1459) | |
| | | <221> cloning vector | |
| | | <222> (1468) . . . (1858) | |
| 566 | intron-pro-FIX | <221> intron | intron-propeptide-mature human FIX |
| | optimized | <222> (1) . . . (163) | R318Y/R338E/T343R optimized 3 |
| | | <221> propeptide | (low CpG) |
| | | <222> (164) . . . (214) | |
| | | <221> exon | |
| | | <222> (215) . . . (1459) | |
| | | <221> cloning vector | |
| | | <222> (1468) . . . (1857) | |
| 572 | SEQ ID NO: | amp: 7021-7125 + 1-20 | AAV-hFIX (R318Y/R338E/T343R) |
| | 451 vector with | ColE1/pMB1/pBR322/pUC | optimized 1 |
| | Pro-FIX from | ori Rep: 572-1160 | |
| | SEQ ID NO: | CAP binding site: 1448-1469 | |
| | 562/569 | lac operon promoter: 1484- | |
| | (optimized 1) | 1514 | |
| | | Left ITR: 1597-1715 | |
| | | ApoE/C1 gene locus | |
| | | (enhancer): 1781-2101 | |
| | | huSerpin A antitrypsin liver | |
| | | promoter (partial): 2111-2503 | |
| | | kozak: 2598-2603 | |
| | | FIX Signal Sequence: 2604- | |
| | | 2687 | |
| | | FIX Propeptide: 2688-2690; | |
| | | 4129-4179 | |
| | | FIX partial intron: 2691-4128 | |
| | | FIX Propeptide: 4129-4179 | |
| | | (from new seq) | |
| | | FIX Mature: 4180-5424 | |
| | | bGH poly(A) signal: 5539- | |
| | | 5763 | |
| | | Right ITR: 5877-6006 | |
| 573 | SEQ ID NO: | amp: 7021-7125 + 1-20 | AAV-hFIX (R318Y/R338E/T343R) |
| | 451 vector with | ColE1/pMB1/pBR322/pUC | optimized 2 |
| | Pro-FIX from | ori Rep: 572-1160 | |
| | SEQ ID NO: | CAP binding site: 1448-1469 | |
| | 563/570 | lac operon promoter: 1484- | |
| | (optimized 2) | 1514 | |
| | | Left ITR: 1597-1715 | |
| | | ApoE/C1 gene locus | |
| | | (enhancer): 1781-2101 | |
| | | huSerpin A antitrypsin liver | |
| | | promoter (partial): 2111-2503 | |
| | | kozak: 2598-2603 | |
| | | FIX Signal Sequence: 2604- | |
| | | 2687 | |
| | | FIX Propeptide: 2688-2690; | |
| | | 4129-4179 | |
| | | FIX partial intron: 2691-4128 | |
| | | FIX Propeptide: 4129-4179 | |
| | | (from new seq) | |
| | | FIX Mature: 4180-5424 | |
| | | bGH poly(A) signal: 5539- | |
| | | 5763 | |
| | | Right ITR: 5877-6006 | |
| 574 | SEQ ID NO: | amp: 7021-7125 + 1-20 | AAV-hFIX (R318Y/R338E/T343R) |
| | 451 vector with | ColE1/pMB1/pBR322/pUC | optimized 3 |
| | Pro-FIX from | ori Rep: 572-1160 | |
| | SEQ ID NO: | CAP binding site: 1448-1469 | |
| | 564/571 | lac operon promoter: 1484- | |
| | (optimized 3) | 1514 | |
| | | Left ITR: 1597-1715 | |
| | | ApoE/C1 gene locus | |
| | | (enhancer): 1781-2101 | |
| | | huSerpin A antitrypsin liver | |
| | | promoter (partial): 2111-2503 | |
| | | kozak: 2598-2603 | |
| | | FIX Signal Sequence: 2604- | |
| | | 2687 | |
| | | FIX Propeptide: 2688-2690; | |
| | | 4129-4179 | |
| | | FIX partial intron: 2691-4128 | |
| | | FIX Propeptide: 4129-4179 | |
| | | (from new seq) | |
| | | FIX Mature: 4180-5424 | |
| | | bGH poly(A) signal: 5539- | |
| | | 5763 | |
| | | Right ITR: 5877-6006 | |
| 575 | SEQ ID NO: | amp: 7021-7125 + 1-20 | AAV-human FIX |
| | 451 vector with | ColE1/pMB1/pBR322/pUC | R318Y/R338E/T343R optimized |
| | Pro-FIX from | ori Rep: 572-1160 | |
| | SEQ ID NO: | CAP binding site: 1448-1469 | |
| | 566 (optimized) | lac operon promoter: 1484- | |
| | | 1514 | |
| | | Left ITR: 1597-1715 | |
| | | ApoE/C1 gene locus | |
| | | (enhancer): 1781-2101 | |
| | | huSerpin A antitrypsin liver | |
| | | promoter (partial): 2111-2503 | |
| | | kozak: 2598-2603 | |
| | | FIX Signal Sequence: 2604- | |
| | | 2687 | |
| | | FIX Propeptide: 2688-2690; | |
| | | 4129-4179 | |
| | | FIX partial intron: 2691-4128 | |
| | | FIX Propeptide: 4129-4179 | |
| | | FIX Mature: 4180-5424 | |
| | | bGH poly(A) signal: 5539- | |
| | | 5763 | |
| | | Right ITR: 5877-6006 | |

Since modifications will be apparent to those of skill in this art, it is intended that this invention be limited only by the scope of the appended claims.

1. An adeno-associated viral (AAV) vector comprising:
a capsid that transduces human hepatocytes with greater transduction efficiency, or to a greater extent with a lower amount of AAV introduced, compared to the AAV capsid designated DJ/8 (SEQ ID NO: 427, capsid polypeptide); and
a nucleic acid construct that encodes a modified Factor IX (FIX) polypeptide encapsulated in the capsid, wherein:
the nucleic acid construct comprises inverted terminal repeats (ITRs) from an adeno-associated virus, flanking nucleic acid encoding the modified FIX polypeptide;
the nucleic acid construct encoding the modified FIX polypeptide comprises all or a portion of a FIX gene intron, whereby expression of the encoded FIX polypeptide is increased in a human cell or a human compared to expression of the FIX polypeptide without the intron;
the encoded modified FIX polypeptide comprises one or more of an insertion, deletion, and replacement of amino acid(s) in an unmodified FIX polypeptide;
the unmodified FIX polypeptide comprises a sequence of amino acid residues set forth in any of SEQ ID NOs: 2, 3, 20 and 325, or is a catalytically active fragment thereof; and
the encoded modified FIX polypeptide, when in activated form, has coagulation activity of at least about 7 times the coagulation activity of the activated form of wild-type FIX of SEQ ID NO:3 or SEQ ID NO:20.

2. The AAV vector of claim 1, wherein the capsid that transduces hepatocytes is a chimeric capsid comprising a mixture of sequences from two or more of AAV1, AAV6, AAV3B, and AAV8; and
the vector transduces hepatocytes with greater efficiency than each of AAV1, AAV6, AAV3B, and AAV8.

3. The AAV vector of claim 1, wherein the intron is all or a portion of the first intron of human FIX (hFIX), wherein:
the portion is sufficient to increase expression of the encoded FIX polypeptide in a human cell or a human to whom the construct is administered for gene therapy; and
the first intron of FIX comprises the sequence of nucleotides set forth in SEQ ID NO:434, or a sequence having at least 95% sequence identity therewith.

4. The AAV vector of claim 3, wherein the portion comprises at least 10%, at least 12%, at least 15%, at least 16%, or at least 20% of the intron or of the sequence having at least 98% sequence identity with the intron, whereby expression of the encoded FIX polypeptide is increased in a human cell or a human compared to expression of the FIX polypeptide without the intron.

5. The AAV vector of claim 1, wherein the intron comprises the sequence of nucleotides set forth in SEQ ID NO:433, or a sequence having at least 98% sequence identity therewith, or the intron consists of the sequence of nucleotides set forth in SEQ ID NO:433.

6. The AAV vector of claim 1, wherein:
the intron or portion thereof is inserted between the nucleotides encoding amino acid residues corresponding to residues 29 and 30 of the unmodified FIX polypeptide of SEQ ID NO:2; and
corresponding residues are identified by alignment with SEQ ID NO:2 or with SEQ ID NO:3.

7. The AAV vector of claim 1, wherein the encoded modified FIX polypeptide, when in activated form, has greater than 20-fold activity compared to the activity of the activated form of wild-type FIX of SEQ ID NO: 2, 3, 20, or 325.

8. The AAV vector of claim 1, wherein the construct comprises nucleic acid encoding the FIX signal sequence, the first residue of the propeptide, the intron portion, and the remaining residues of the FIX polypeptide, including the remaining residues of the propeptide and mature FIX polypeptide.

9. The AAV vector of claim 1, wherein:
the construct, with reference to the unmodified FIX polypeptide of SEQ ID NO:2, comprises, in the following order: nucleic acid encoding the signal sequence, residue 1 of the propeptide, the intron or portion thereof, residues 2-18 of the propeptide, and residues corresponding to residues 47-461 of the mature FIX polypeptide; and
residue positions are determined by alignment with SEQ ID NO:2.

10. The AAV vector of claim 1, wherein:
the modified FIX polypeptide comprises:
an amino acid replacement T343R, T343E, or T343D, or the same replacement at a corresponding amino acid residue in an unmodified FIX polypeptide; and
an amino acid replacement R338E or R338D, or the same replacement at a corresponding amino acid residue in an unmodified FIX polypeptide; wherein:
the unmodified FIX polypeptide comprises the sequence of amino acids set forth in SEQ ID NO: 2, 3, 20, or 325, or a catalytically active fragment thereof; and
amino acid residue positions are referenced by mature numbering, and identified by alignment with SEQ ID NO:3.

11. The AAV vector of claim 10, wherein:
the modified FIX polypeptide comprises amino acid replacements corresponding to R338E/T343R; and
the unmodified FIX polypeptide comprises the sequence of amino acids set forth in SEQ ID NO:2, 3, 20, or 325, or a catalytically active fragment thereof.

12. The AAV vector of claim 11, wherein:
the modified FIX polypeptide comprises an amino acid replacement corresponding to R318Y, whereby the FIX polypeptide comprises amino acid replacements corresponding to R318Y/R338E/T343R; and
the unmodified FIX polypeptide comprises the sequence of amino acids set forth in SEQ ID NO: 2, 3, 20, or 325, or a catalytically active fragment thereof.

13. The AAV vector of claim 12, wherein:
the modified FIX polypeptide comprises the sequence of amino acids set forth in SEQ ID NO:394 or an activated two-chain FIXa form thereof; or
the modified FIX polypeptide comprises the sequence of amino acids set forth in SEQ ID NO:394 in which residue 148 is A (alanine) as set forth in SEQ ID NO:490, or is an activated two-chain FIXa form thereof; or
the modified FIX polypeptide comprises a sequence having at least 95% sequence identity with the sequence of amino acids set forth in SEQ ID NO:394 or an activated two-chain FIXa form thereof, and containing the amino acid replacements corresponding to R318Y/R338E/T343R.

14. The AAV vector of claim 1, wherein:
the modified FIX polypeptide comprises the amino acid replacement corresponding to R338L; and
the unmodified FIX polypeptide comprises the sequence of amino acids set forth in SEQ ID NO: 2, 3, 20, or 325, or a catalytically active fragment thereof.

15. The AAV vector of claim 1, wherein:
the modified FIX polypeptide comprises amino acid replacements selected from among the replacements R318Y/R338E/R403E/E410N, R318Y/R338E/T343R/R403E/E410N, R318Y/R338E/T343R/E410N, Y155F/R318Y/R338E/T343R/R403E, Y155F/K228N/K247N/N249S/R318Y/R338E/T343R/R403E/E410N, Y155F/K247N/N249S/R318Y/R338E/T343R/R403E, R318Y/R338E/T412A, K247N/N249S/R318Y/R338E/T343R/R403E, R318Y/R338E/T343R, Y155F/K247N/N249S/R318Y/R338E/T343R, K228N/R318Y/R338E/T343R/R403E/E410N, K228N/K247N/N249S/R318Y/R338E/T343R/R403E, R318Y/R338E/T343R/R403E/E410S, Y155F/K247N/N249S/R318Y/R338E, K247N/N249S/R318Y/R338E/T343R, R318Y/T343R/E410N, Y155F/R318Y/R338E/R403E, R318Y/R338E/R403E, R318Y/R338E/E410N, K228N/R318Y/E410N, R318Y/R403E/E410N, D203N/F205T/R318Y/E410N, A103N/N105S/R318Y/R338E/R403E/E410N, D104N/K106S/R318Y/R338E/R403E/E410N, K228N/R318Y/R338E/R403E/E410N, I251S/R318Y/R338E/R403E/E410N, D104N/K106S/I251S/R318Y/R338E/R403E/E410N, D104N/K106S/R318Y/R338E/E410N, I251S/R318Y/E410N/R338E, D104N/K106S/I251S/R318Y/R338E/E410N, A103N/N105S/K247N/N249S/R318Y/R338E/R403E/E410N, D104N/K106S/K247N/N249S/R318Y/R338E/R403E/E410N, K228N/K247N/N249S/R318Y/R338E/R403E/E410N, A103N/N105S/Y155F/R318Y/R338E/R403E/E410N, D104N/K106S/Y155F/R318Y/R338E/R403E/E410N, Y155F/K228N/R318Y/R338E/R403E/E410N, Y155F/I251S/R318Y/R338E/R403E/E410N, K247N/N249S/R318Y/R338E/R403E/E410N, Y155F/R318Y/R338E/R403E/E410N, K247N/N249S/R318Y/R338E/E410N, Y155F/R318Y/R338E/E410N, Y155F/K247N/N249S/R318Y/R338E/E410N, R318Y/R338E/R403E/E410S, R318Y/R338E/R403E/E410N/T412V, R318Y/R338E/R403E/E410N/T412A, R318Y/R338E/R403E/T412A, R318Y/R338E/E410S, R318Y/R338E/T412A, R318Y/R338E/E410N/T412V, D85N/K228N/R318Y/R338E/R403E/E410N, N260S/R318Y/R338E/R403E/E410N, R318Y/R338E/N346D/R403E/E410N, Y155F/R318Y/R338E/N346D/R403E/E410N, K247N/N249S/N260S/R318Y/R338E/R403E/E410N, D104N/K106S/N260S/R318Y/R338E/R403E/E410N, Y155F/N260S/R318Y/R338E/R403E/E410N, Y155F/R318Y/R338E/T343R/R403E/E410N, D104N/K106S/Y155F/K247N/N249S/R318Y/R338E/R403E/E410N, D104N/K106S/K228N/K247N/N249S/R318Y/R338E/R403E/E410N, Y155F/K228N/K247N/N249S/R318Y/R338E/R403E/E410N, Y155F/K247N/N249S/N260S/R318Y/R338E/R403E/E410N, D104N/K106S/R318Y/R338E/T343R/R403E/E410N, R318Y/R338E/N346Y/R403E/E410N, R318Y/R338E/T343R/N346Y/R403E/E410N, R318Y/R338E/T343R/N346D/R403E/E410N, R318Y/R338E/Y345A/R403E/E410N, K228N/I251S/R318Y/R338E/R403E/E410N, R318Y/R338E/Y345A/N346D/R403E/E410N, Y155F/K247N/N249S/R318Y/R338E/R403E, K247N/N249S/R318Y/R338E/R403E, Y155F/K247N/N249S/R318Y/R403E/E410N, K247N/N249S/R318Y/R403E/E410N, R318Y/R338E/T343R/R403E, Y155F/R318Y/R338E/T343R/E410N, R318Y/T343R/R403E/E410N, Y155F/R318Y/T343R/R403E/E410N, Y155F/K247N/N249S/R318Y/R338E/T343R/R403E/E410N, K247N/N249S/R318Y/R338E/T343R/R403E/E410N, Y155F/K228N/I251S/R318Y/R338E/R403E/E410N, N260S/R318Y/R338E/T343R/R403E/E410N, Y155F/N260S/R318Y/R338E/T343R/R403E/E410N, K228N/K247N/N249S/R318Y/R338E/T343R/R403E/E410N, Y155F/K247N/N249S/R318Y/R403E, Y155F/K247N/N249S/R318Y/E410N, K247N/N249S/R318Y/R338E/T343R/E410N, Y155F/K247N/N249S/R318Y/R338E/T343R/E410N, Y155F/K247N/N249S/R318Y/T343R/R403E/E410N, K247N/N249S/R318Y/T343R/R403E/E410N, Y155F/K247N/N249S/R318Y/T343R/R403E, K247N/N249S/R318Y/T343R/R403E, Y155F/K247N/N249S/R318Y/T343R/E410N, K247N/N249S/R318Y/T343R/E410N, Y155F/R318Y/R338E/T343R, Y155F/R318Y/T343R/R403E, Y155F/R318Y/T343R/E410N, K228N/K247N/N249S/R318Y/R338E/T343R/E410N, Y155F/K247N/N249S/R318Y/T343R, Y155F/K247N/N249S/R318Y/R338E/R403E/E410N, K228N/K247N/N249S/R318Y/T343R/R403E/E410N, and Y155F/R318Y/R403E/E410N, or the same replacements at corresponding amino acid residues in an unmodified FIX polypeptide; wherein:
numbering is with respect to the mature FIX polypeptide of SEQ ID NO:3; and
the unmodified FIX polypeptide comprises the sequence of amino acids set forth in SEQ ID NO: 2, 3, 20, or 325, or a catalytically active fragment thereof.

16. The AAV vector of claim 1, wherein:
the encoded modified FIX polypeptide comprises the amino acid replacement R338L;
amino acid residue numbering is with respect to the mature FIX polypeptide of SEQ ID NO:3; and
the unmodified FIX polypeptide comprises the sequence of amino acids set forth in SEQ ID NO: 2, 3, 20, or 325, or a catalytically active fragment thereof.

17. The AAV vector of claim 1, wherein:
the mature portion of FIX, corresponding to residues 1-415 of SEQ ID NO:3, is encoded by the sequence of nucleotides set forth in any of SEQ ID NOs:483-487; and
the portion encoding the modified FIX polypeptide and the intron comprises the sequence of nucleotides set forth in any of SEQ ID NOs:462-467.

18. The AAV vector of claim 1, wherein the codons are optimized for expression in a human cell or a mouse cell.

19. The AAV vector of claim 18, wherein:
a) codon optimized sequences are set forth in any of SEQ ID NOs:518-520, wherein SEQ ID NOs:518-520 are as follows:

| | | |
|---|---|---|
| 518 | encodes 47-461 | hWT-FIX mature codon optimized with |
| | | a stringency of 0.5 optimization; |
| 519 | encodes 47-461 | hWT-FIX-mature codon optimized with |
| | | a stringency 0.8 optimization; and |
| 520 | encodes 47-461 | hWT-FIX-mature codon optimized with |
| | | a stringency 1.0 optimization; or |

b) the nucleic acid encoding the mature modified FIX polypeptide, corresponding to residues 47-461 of SEQ ID NO:2, and residues 1-415 of SEQ ID NO:3, has the sequence of nucleotides set forth in any of SEQ ID NOs:521-526, wherein SEQ ID NOs:521-526 are as follows:

| | | |
|---|---|---|
| 521 | encodes 47-461 | human FIX R318Y/R338E/T343R |
| | | mature codon optimized with a |
| | | stringency of 0.5 optimization; |
| 522 | encodes 47-461 | human FIX R318Y/R338E/T343R |
| | | mature codon optimized with a |
| | | stringency of 0.8 optimization; |
| 523 | encodes 47-461 | human FIX R318Y/R338E/T343R |
| | | mature codon optimized with a |
| | | stringency of 1.0 optimization; |
| 524 | encodes 47-461 | human FIX R338L mature codon |
| | | optimized with a stringency of |
| | | 0.5 optimization; |
| 525 | encodes 47-461 | human FIX R338L mature codon |
| | | optimized with as tringency |
| | | of 0.8 optimization; and |
| 526 | encodes 47-461 | human FIX R338L mature codon |
| | | optimized with a stringency of |
| | | 1.0 optimization; or |

c) the nucleic acid sequence corresponding to the nucleic acid encoding residues 30-461 of SEQ ID NO:2 has the sequence of nucleotides set forth in any of SEQ ID NOs:527-529, wherein SEQ ID NOs:527-529 are as follows:

| | | |
|---|---|---|
| 527 | encodes 30-461 | hWT-FIX codon optimized with |
| | | a stringency of 0.5 optimization; |
| 528 | encodes 30-461 | hWT-FIX codon optimized with a |
| | | stringency of 0.8 optimization; and |
| 529 | encodes 30-461 | hWT-FIX codon optimized with a |
| | | stringency of 1.0 optimization; or |

d) the nucleic acid encoding the modified FIX polypeptide, corresponding to residues 30-461 of SEQ ID NO:2, comprises the sequence of nucleotides set forth in any of SEQ ID NOs:530-535, wherein SEQ ID NOs:530-535 are as follows:

| | | |
|---|---|---|
| 530 | encodes 30-461 | human FIX R318Y/R338E/T343R codon |
| | | optimized with a stringency of |
| | | 0.5 optimization; |
| 531 | encodes 30-461 | human FIX R318Y/R338E/T343R codon |
| | | optimized with a stringency of |
| | | 0.8 optimization; |
| 532 | encodes 30-461 | human FIX R318Y/R338E/T343R codon |
| | | optimized with a stringency of |
| | | 1.0 optimization; |
| 533 | encodes 30-461 | human FIX R338L codon optimized with |
| | | a stringency of 0.5 optimization; |
| 534 | encodes 30-461 | human FIX R338L codon optimized with |
| | | a stringency of 0.8 optimization; and |
| 535 | encodes 30-461 | human FIX R338L codon optimized with |
| | | a stringency of 1.0 optimization; or |

e) the nucleic acid encoding the signal sequence, the partial intron, and the modified human FIX polypeptide comprises the sequence of nucleotides set forth in any of SEQ ID NOs:536-553, wherein SEQ ID NOs:536-553 are as follows:

| | | |
|---|---|---|
| 536 | precursor + intron | hFIXss-Pro-mini Intron-Pro-human wild type FIX optimized 0.5; |
| 537 | precursor + intron | hFIXss-Pro-mini Intron-Pro-human wild type FIX optimized 0.8; |
| 538 | precursor + intron | hFIXss-Pro-mini Intron-Pro-human wild type FIX optimized 1.0; |
| 539 | precursor + intron | hFIXss-Pro-mini Intron-Pro-human FIX R318Y/R338E/T343R |
| | | optimized 0.5; |
| 540 | precursor + intron | hFIXss-Pro-mini Intron-Pro-human FIX R318Y/R338E/T343R |
| | | optimized 0.8; |
| 541 | precursor + intron | hFIXss-Pro-mini Intron-Pro-human FIX R318Y/R338E/T343R |
| | | optimized 1.0; |
| 542 | precursor + intron | hFIXss-Pro-mini Intron-Pro-human FIX R338L optimized 0.5; |
| 543 | precursor + intron | hFIXss-Pro-mini Intron-Pro-human FIX R338L optimized 0.8; |
| 544 | precursor + intron | hFIXss-Pro-mini Intron-Pro-human FIX R338L optimized 1.0; |
| 545 | precursor + intron | mouse optimized hFIXss-Pro-mini Intron-Pro-human wild type |
| | | FIX optimized 0.5; |
| 546 | precursor + intron | mouse optimized hFIXss-Pro-mini Intron-Pro-human wild type |
| | | FIX optimized 0.8; |
| 547 | precursor + intron | mouse optimized hFIXss-Pro-mini Intron-Pro-human wild type |
| | | FIX optimized 1.0; |
| 548 | precursor + intron | mouse optimized hFIXss-Pro-mini Intron-Pro-human FIX |
| | | R318Y/R338E/T343R optimized 0.5; |
| 549 | precursor + intron | mouse optimized hFIXss-Pro-mini Intron-Pro-human FIX |
| | | R318Y/R338E/T343R optimized 0.8; |
| 550 | precursor + intron | mouse optimized hFIXss-Pro-mini Intron-Pro-human FIX |
| | | R318Y/R338E/T343R optimized 1.0; |
| 551 | precursor + intron | mouse optimized hFIXss-Pro-mini Intron-Pro-human FIX R338L |
| | | optimized 0.5; |
| 552 | precursor + intron | mouse optimized hFIXss-Pro-mini Intron-Pro-human FIX R338L |
| | | optimized 0.8; and |
| 553 | precursor + intron | mouse optimized hFIXss-Pro-mini Intron-Pro-human FIX R338L |
| | | optimized 1.0. |

20. The AAV vector of claim 18, wherein the cell is a human liver cell.

21. The AAV vector of claim 18, wherein the portion encoding the modified FIX polypeptide and the intron comprises the sequence of nucleotides set forth in SEQ ID NO:466.

22. The AAV vector of claim 21, comprising 2 ITRs flanking the nucleic acid encoding the modified FIX polypeptide with the intron, wherein each ITR is an AAV ITR, or a chimeric or hybrid AAV ITR.

23. The AAV vector of claim 22, wherein an ITR is selected from among:
an ITR that comprises the sequence of nucleotides set forth in SEQ ID NO: 435 or 437, or ITRs encoded by nucleotides 1-119 and 4281-4410 of SEQ ID NO:456, or an ITR encoded by any of SEQ ID NOs:436 and 501-511.

24. The AAV vector of claim 1, comprising transcription regulatory sequences operatively linked to the nucleic acid, for transcription of the nucleic acid encoding the modified FIX polypeptide.

25. The AAV vector of claim 24, wherein the regulatory sequences are liver-specific.

26. The AAV vector of claim 1, comprising a promoter, and optionally, additional regulatory sequences operatively linked for expression of the nucleic acid encoding the modified FIX polypeptide.

27. The AAV vector of claim 26, wherein the promoter is a liver-specific or hepatocyte-specific promoter.

28. The AAV vector of claim 1, comprising the sequence of nucleotides set forth in any of SEQ ID NOs: 456-461 and 572-575.

29. The AAV vector of claim 1, comprising codons that are optimized for expression in a human cell, wherein the modified FIX polypeptide is encoded by the sequence of nucleotides set forth in any of SEQ ID NOs:569-571, or the intron-propeptide-mature human FIX polypeptide comprising the replacements R318Y/R338E/T343R is encoded by the sequence of nucleotides set forth in any of SEQ ID NOs:562-566.

30. The AAV vector of claim 1, comprising the sequence of nucleotides set forth in SEQ ID NO:460 or as set forth in SEQ ID NO:460, except where the modified FIX polypeptide is encoded by the sequence of nucleotides set forth in any of SEQ ID NOs:569-571, or except where the intron-propeptide-modified FIX polypeptide is encoded by the sequence of nucleotides set forth in any of SEQ ID NOs:562-566.

31. The AAV vector of claim 1 that comprises a sequence of nucleotides set forth in any of SEQ ID NOs:447-455, except where the modified FIX polypeptide is encoded by the sequence of nucleotides set forth in any of SEQ ID NOs:569-571, or except where the intron-propeptide-modified FIX polypeptide is encoded by the sequence of nucleotides set forth in any of SEQ ID NOs:562-566.

32. The AAV vector of claim 1, wherein the capsid that transduces hepatocytes transduces human and mouse hepatocytes with substantially the same efficiency or to the same extent.

33. The AAV vector of claim 1, wherein the capsid that transduces hepatocytes also transduces islet cells.

34. The AAV vector of claim 1, wherein the capsid comprises the sequence of amino acids set forth in any of SEQ ID NOs:418-420 and 492-500, or a sequence having at least 95% sequence identity therewith.

35. The AAV vector of claim 1, wherein the capsid is designated KP-1, KP-2, or KP-3.

36. The AAV vector of claim 1, wherein the capsid is encoded by the sequence of nucleotides set forth in any of SEQ ID NOs:421-423, or a sequence having at least 95% sequence identity therewith.

37. The AAV vector of claim 36 that encodes a modified FIX polypeptide comprising the sequence of amino acids set forth in SEQ ID NO:394, or in SEQ ID NO:394 in which residue 148 is A as set forth in SEQ ID NO:490.

38. The AAV vector of claim 1, comprising the nucleic acid construct whose sequence is set forth in SEQ ID NO:460 or in SEQ ID NO:461.

39. An AAV vector comprising:
a capsid designated LK03 of SEQ ID NO:429 or 561, or a capsid having at least 95% sequence identity therewith; and
a nucleic acid construct that encodes a modified FIX polypeptide encapsulated in the capsid, wherein:
the nucleic acid construct comprises inverted terminal repeats (ITRs) from an adeno-associated virus, flanking nucleic acid encoding the modified FIX polypeptide;
the nucleic acid construct encoding the modified FIX polypeptide comprises all or a portion of a FIX gene intron;
the intron is all or a portion of the first intron of human FIX (hFIX);
the intron comprises the sequence of nucleotides set forth in SEQ ID NO:433, or a sequence having at least 98% sequence identity therewith;
the portion of the intron is sufficient to increase expression of the encoded modified FIX polypeptide in a human cell or a human to whom the construct is administered for gene therapy;
the encoded modified FIX polypeptide comprises one or more of an insertion, deletion, and replacement of amino acids in an unmodified FIX polypeptide;
the insertion(s), deletion(s), and replacement(s) of amino acids comprise amino acid replacements that correspond to R338E/T343R, or R318Y, or R318Y/R338E/T343R;
corresponding residues are identified by alignment with SEQ ID NO:2 or SEQ ID NO:3;
the unmodified FIX polypeptide comprises the sequence of amino acids set forth in any of SEQ ID NOs: 2, 3, 20, and 325, or a catalytically active fragment thereof; and
the encoded modified FIX polypeptide, when in activated form, has coagulation activity of at least about 7 times the coagulation activity of the activated form of wild-type FIX of SEQ ID NO:3 or SEQ ID NO:20.

40. The AAV vector of claim 39, wherein the intron consists of the sequence of nucleotides set forth in SEQ ID NO:433.

41. The AAV vector of claim 39, wherein:
the intron or portion thereof is inserted between the nucleotides encoding amino acid residues corresponding to residues 29 and 30 of the unmodified FIX polypeptide of SEQ ID NO:2; and
corresponding residues are identified by alignment with SEQ ID NO:2 or SEQ ID NO:3.

42. The AAV vector of claim 39, wherein the construct, with reference to the unmodified FIX polypeptide of SEQ ID NO:2, comprises, in the following order: nucleic acid encoding the signal sequence, residue 1 of the propeptide, the intron or portion thereof, residues 2-18 of the propeptide, and residues corresponding to residues 47-461 of the mature FIX polypeptide, wherein residue positions are determined by alignment with SEQ ID NO:2.

43. The AAV vector of claim 42, wherein the encoded modified FIX polypeptide comprises the sequence of amino acids set forth in SEQ ID NO:394.

44. The AAV vector of claim 43, wherein the modified FIX polypeptide is encoded by the sequence of nucleotides set forth in any of SEQ ID NOs: 569-571.

45. The AAV vector of claim 39, wherein the capsid comprises the sequence of amino acids set forth in SEQ ID NO:429 or 561.

46. A nucleic acid construct, comprising inverted terminal repeats (ITRs) from an adeno-associated virus, flanking nucleic acid encoding a modified FIX polypeptide, wherein:
the construct comprises all or a portion of the FIX first gene intron, wherein:
the first intron of FIX comprises the sequence of nucleotides set forth in SEQ ID NO:434, or a sequence having at least 95% sequence identity therewith; and
the portion is sufficient to increase expression of the encoded modified FIX polypeptide in a human cell or a human to whom the construct is administered for gene therapy;
the intron is inserted after or downstream from nucleic acid encoding the signal sequence in the nucleic acid encoding the modified FIX polypeptide;
the encoded modified FIX polypeptide comprises one or more of an insertion, deletion, and replacement of amino acids;
the insertion(s), deletion(s), or replacement(s) comprise R318Y, or R338E/T343R, or R338L, or combinations thereof;
corresponding residues are identified by alignment with SEQ ID NO:2 or with SEQ ID NO:3;
the unmodified FIX polypeptide comprises the sequence of amino acids set forth in any of SEQ ID NOs: 2, 3, 20, and 325, or is a catalytically active fragment thereof; and
the encoded modified FIX polypeptide, when in activated form, has coagulation activity of at least about 7 times the coagulation activity of the activated form of wild-type FIX of SEQ ID NO:3 or SEQ ID NO:20.

47. The nucleic acid construct of claim 46, wherein:
the portion of the intron comprises at least 15%, at least 16%, or at least 20% of the intron, or of a sequence having at least 95% or 98% sequence identity therewith, whereby expression of the encoded modified FIX polypeptide is increased in a human cell or a human compared to expression of the modified FIX polypeptide without the intron; and
the first intron of FIX comprises the sequence of nucleotides set forth in SEQ ID NO:434.

48. The nucleic acid construct of claim 46 that comprises nucleic acid encoding the FIX signal sequence, the first residue of the propeptide, the intron, and the remaining residues of the modified FIX polypeptide, including the remaining residues of the propeptide and mature FIX polypeptide.

49. The nucleic acid construct of claim 46, wherein the construct, with reference to the unmodified FIX polypeptide of SEQ ID NO:2, comprises the signal sequence (residues 1-28), residue 1 of the propeptide, the intron, residues 2-18 of the propeptide, and residues corresponding to residues 47-461 of the mature FIX polypeptide, wherein residue positions are determined by alignment with SEQ ID NO:2.

50. The nucleic acid construct of claim 46, wherein the modified FIX polypeptide comprises the sequence of amino acids set forth in SEQ ID NO:394, or SEQ ID NO:394 in which residue 148 is A as set forth in SEQ ID NO:490, or a sequence having at least 95% sequence identity with the sequence of amino acids set forth in SEQ ID NO:394 and containing the replacements corresponding to R318Y/R338E/T343R.

51. The nucleic acid construct of claim 46, wherein:
the mature portion of FIX, corresponding to residues 1-415 of SEQ ID NO:3, is encoded by the sequence of nucleotides set forth in any of SEQ ID NOs:483-487; or
the portion encoding the modified FIX polypeptide and the intron comprises the sequence of nucleotides set forth in any of SEQ ID NOs:462-467.

52. The nucleic acid construct of claim 46, wherein the codons encoding the modified FIX polypeptide are optimized for expression in a human cell or a mouse cell.

53. The nucleic acid construct of claim 46, wherein the nucleotides encoding the modified FIX polypeptide comprise the sequence of nucleotides set forth in any of SEQ ID NOs:569-571.

54. The nucleic acid construct of claim 46, wherein the nucleotides encoding the modified FIX polypeptide comprise the sequence of nucleotides set forth in any of SEQ ID NOs:562-566.

55. The nucleic acid construct of claim 46, wherein the portion encoding the modified FIX polypeptide and the partial intron comprises the sequence of nucleotides set forth in SEQ ID NO:466.

56. A vector, comprising the nucleic acid construct of claim 46, wherein the vector comprises the sequence of nucleotides of any of SEQ ID NOs:448-455, or a sequence having at least 95% sequence identity therewith that encodes the modified FIX polypeptide that comprises the amino acid replacements that correspond to R338E/T343R, or R318Y, or R318Y/R338E/T343R.

57. A pharmaceutical composition, comprising the AAV vector of claim 1 in a pharmaceutically acceptable vehicle.

58. The pharmaceutical composition of claim 57, wherein the AAV vector encodes a modified FIX polypeptide comprising the sequence of amino acids set forth in SEQ ID NO:394, or SEQ ID NO:394 in which residue 148 is A as set forth in SEQ ID NO:490.

59. The pharmaceutical composition of claim 57 that is formulated for systemic administration.

60. The pharmaceutical composition of claim 57 that is formulated for intravenous administration or is formulated for direct administration into the liver.

61. A method of treating hemophilia, comprising administering the AAV vector of claim 1 to a subject who has hemophilia.

62. The method of claim 61, wherein the AAV vector comprises the capsid designated KP-1, KP-2, KP-3, or LK-03.

63. The method of claim 61, wherein the subject has hemophilia B.

64. The method of claim 62, wherein the modified FIX polypeptide encoded in the AAV vector comprises the sequence of amino acids set forth in SEQ ID NO:394, or SEQ ID NO:394 in which residue 148 is A as set forth in SEQ ID NO:490.

65. The method of claim 61, wherein the AAV vector is administered intravenously, or intramuscularly, or by direct injection into the liver.

66. A method of producing an AAV vector encoding a modified FIX polypeptide, comprising packaging the nucleic acid construct of claim 46 into a capsid comprising the sequence of amino acids set forth in any of SEQ ID NOs:418-420 and 492-500.

67. The method of claim 66, wherein the encoded modified FIX polypeptide comprises the sequence of amino acids set forth in SEQ ID NO:394, or SEQ ID NO:394 in which residue 148 is A as set forth in SEQ ID NO:490.

68. The AAV vector of claim 1, wherein the modified FIX polypeptide contains up to 10 amino acid modifications, or has at least 95% sequence identity to the unmodified FIX polypeptide of any of SEQ ID NOs: 2, 3, 20, and 325.
